(19) United States
(12) Patent Application Publication
(10) Pub. No.: US 2006/0276339 A1
(43) Pub. Date: Dec. 7, 2006

(54) Methods and compositions for increasing the efficacy of biologically-active ingredients

(76) Inventors: J. Brian Windsor, Austin, TX (US); Stan J. Roux, Austin, TX (US); Alan M. Lloyd, Austin, TX (US); Collin E. Thomas, Dallas, TX (US)

Correspondence Address: FULBRIGHT & JAWORSKI L.L.P., 600 CONGRESS AVE., SUITE 2400, AUSTIN, TX, 78701, US

(21) Appl. No.: 10/531,744

(22) PCT Filed: Oct. 16, 2003
(86) PCT No.: PCT/US03/32667

§ 371 Date: Jan. 23, 2006

Related U.S. Application Data
(60) Provisional application No. 60/418,803, filed on Oct. 16, 2002.

(51) Int. Cl. A01N 57/00; A61K 31/43; A61K 31/545; A61K 31/425; A61K 31/405; A61K 31/165; A01N 43/16; A01N 47/06; A61K 31/235; A61K 31/24; A61K 31/18; A61K 31/426

(52) U.S. Cl. 504/127; 504/128; 514/192; 514/200; 514/370; 514/602; 514/414; 514/617; 514/615; 514/532; 514/616; 514/535; 514/457; 514/443; 514/369; 514/533; 514/512

(57) ABSTRACT

The invention provides methods and compositions for modulating the sensitivity of cells to cytotoxic compounds and other active agents. In accordance with the invention, compositions are provided comprising combinations of ectophosphatase inhibitors and active agents. Active agents include antibiotics, fungicides, herbicides, insecticides, chemotherapeutic agents, and plant growth regulators. By increasing the efficacy of active agents, the invention allows use of compositions with lowered concentrations of active ingredients.

### BACKGROUND OF THE INVENTION

1. Field of the Invention

The present invention relates generally to the fields of cellular biology. More particularly, it concerns methods and compositions for the modulation of cellular sensitivity to biologically-active agents.

2. Description of Related Art

Transport Processes

Cells can use a phenomenon called symport to move soluble products across biological membranes. Symport is a form of coupled movement of two solutes in the same direction across a membrane by a single carrier. Examples of proton and sodium-linked symport systems are found in nearly all living systems. The energetics of the transport event depend on the relative size and electrical nature of the gradient of solutes.

Transport processes have been classified on the basis of their energy-coupling mechanisms. Currently there are four classifications: (1) Primary Active Transport which uses either a chemical, light or electrical energy source, (2) Group Translocation which uses chemical energy sources, (3) Secondary Active Transport which uses either a sodium or proton electrochemical gradient energy source, and (4) Facilitated Diffusion which does not require an energy source (Meyers, 1997). The present invention is related to transport molecules belonging to the first class of transport processes, primary active transport, and therefore, this type of transport will be discussed in further detail.

Primary active transport refers to a process whereby a “primary” source of energy is used to drive the active accumulation of a solute into or extrusion of a solute from a cell. Transport proteins include P-type ATPases and ABC-type ATPases, as well as V-type and E-type ATPases. These types of transport systems are found in both eukaryotes and prokaryotes. The bacterial ABC-type transporters, which are ATP-driven solute pumps, have eukaryotic counterparts. Additionally, many transmembrane solute transport proteins exhibit a common structural motif. The proteins in these families consist of units or domains that pass through the membrane six times, each time as an α-helix. This has led to the suggestion that many transport proteins share a common evolutionary origin, but this is not true of several distinct families of transport proteins.

Numerous structurally distinct bacterial permeases, as well as several homologous eukaryotic transport systems, share a common organization (Meyers, 1997). Two hydrophilic domains or proteins function to couple ATP hydrolysis in the cytoplasm to activate substrate uptake or efflux, and two hydrophobic domains or proteins function as the transmembrane substrate channels. These proteins or protein domains constitute what is referred to as the ABC (ATP-binding cassette) superfamily. Either the two hydrophilic domains or proteins or the two hydrophobic domains or proteins (or both) may exist either as heterodimers or homodimers. If, as in most bacterial systems, each of these constituents is a distinct protein, then either two, three, or four genes will code for them, depending on whether both are homodimers, one is a homodimer and one is a heterodimer, or both are heterodimers, respectively. The best characterized of the eukaryotic proteins included in this family are the multidrug-resistance (MDR) transporter and the cystic fibrosis related chloride ion channel of mammalian cells (cystic fibrosis transmembrane conductance regulator or CFTR) (Meyers, 1997).

Multidrug Resistance

Multidrug resistance (MDR) is a general term that refers to the phenotype of cells or microorganisms that exhibit resistance to different, chemically dissimilar, cytotoxic compounds. MDR can develop after sequential or simultaneous exposure to various drugs. MDR can also develop before exposure to many compounds to which a cell or microorganism may be found to be resistant. MDR which develops before exposure is frequently due to a genetic event which causes the altered expression and/or mutation of an ATP-binding cassette (ABC) transporter (Wadkins and Roepe, 1997). This is true for both eukaryotes and prokaryotes.

One prominent member of the ABC family, P-glycoprotein (Pgp; also known as multidrug resistance protein or MDR1), which is a plasma-membrane glycoprotein that confers a multidrug resistance (MDR) phenotype on cells, is of considerable interest because it provides one mechanism of possibly inhibiting resistance in tumor cells to chemotherapeutic agents (Senior et al., 1995). Pgp is a single polypeptide of ˜1280 amino acids with the typical ABC transporter structure profile. Studies have shown that over expression of Pgp is responsible for the ATP-dependent extrusion of a variety of compounds, including chemotherapeutic drugs, from cells (Abraham et al., 1993).

Over one-hundred ABC transporters have been identified in species ranging from Escherichia coli to humans (Higgins, 1995). For example, the bacteria Lactococcus lactis expresses an ABC transporter, LmrA, which mediates antibiotic resistance by extruding amphiphilic compounds from the inner leaflet of the cytoplasmic membrane (Van Veen et al., 1998). Furthermore, over-expression of LmrA can confer MDR in human lung fibroblasts and LmrA has similar molecular and biochemical properties to Pgp. This demonstrates that bacterial LmrA and Pgp are functionally interchangeable. Additionally, the plant Arabidopsis thaliana encodes an ATP transporter, AtPGP-1, which is a putative Pgp homolog (Dudler and Hertig, 1992). Similarly, the yeast Saccharomyces cerevisiae equivalent of Pgp, STS1 (Bissinger and Kucher, 1994) has been cloned and shown to confer multidrug resistance when over-expressed in yeast, as has the yeast Pdr5p (Kolacskowski et al., 1996). Taken together, these results suggest, that this type of multidrug resistance efflux pump is conserved from bacteria to humans.

While various theories of ABC transporter function have become popular, there is still no precise molecular-level description for the mechanism by which over-expression lowers intracellular accumulation of drugs, in particular how Pgp lowers intracellular accumulation of chemotherapeutic drugs. However, it has been shown that Pgp over-expression also changes plasma membrane electrical potential and intracellular pH which could potentially greatly affect the cellular flux of a large number of compounds to which Pgp confers resistance (Wadkins and Roepe, 1997). Also included in the ABC transporter superfamily are the Cystic Fibrosis Transmembrane Conductance Regulator (CFTR) and the Sulfonyl Urea Receptor (SUR). CFTR and SUR are expressed in the lung epithelium and the β cells of the pancreas, respectively, as well as in other tissues. CFTR functions as a low conductance ATP and cyclic AMP-dependent Cl⁻ channel that also appears to have additional important functions, such as modulation of epithelial NA⁺ regulation of outwardly rectified chloride channels (Wadkins and Roepe, 1997).

Mutations in the CFTR gene produce altered CFTR proteins with defects in CFTR function, leading to profound alterations in epithelial salt transport and altered mucous properties in cystic fibrosis patients that result in chronic lung infections associated with the disease. Id. SUR is triggered by sulfonyl urea drugs to depolarize pancreatic P cells that leads to Ca²⁺ influx, which stimulates fusion of insulin containing vesicles to the plasma membrane. Id. An ATP transporter hypothesis has been suggested for Pgp, CFTR and SUR which theorizes that these ABC transporters function as ATP transport channels (Abraham et al., 1993; Schweibert, 1995; Al-Awgati, 1995). The ATP channel hypothesis, however, has been viewed with skepticism. This is partly due to the inability to show the same results with preparations including purified and reconstituted CFTR, suggesting that the ATP conductance that was originally observed may have been mediated by another protein, not present in the purified system, that is influenced by CFTR (Wadkins and Roepe). There has been no such negative data reported with respect to the ATP charnel hypothesis for Pgp or SUR, but the controversy over CFTR has raised doubt for Pgp and SUR as well.

In support of the ATP channel hypothesis (Huang et al., 1992) have suggested that extracellular ATP leads to elevations in pH, and Weiner et al., (1986) have suggested that extracellular ATP may regulate Na⁻/H⁺ exchange in Ehrlich ascites tumor cells. It has also been observed that changes in Pgp levels affects pH and plasma membrane electrical potentials which could be connected to recent observations suggesting the involvement of ATP transport in MDR.

Additionally, Abraham et al., (1993) have reported that the addition of extracellular ATP to MDR cell lines confers sensitivity to drugs abolishing MDR. The data for this effect were not presented in the article and no further explanation was given for this phenomenon. Furthermore, there have been no subsequent publications addressing or explaining this effect.

Furthermore, Ujhazy et al., (1996) have shown that ecto-5′-nucleotidase is up-regulated in certain MDR cell lines. Ecto-5′-nucleotidase is the final enzyme in the extracellular pathway for salvage of adenosine from phosphorylated purines (Zimmerman, 1992). The proposed hypothesis for the involvement of ecto-5′-nucleotidase in drug resistance considers its role in the maintenance of intracellular ATP pools through the adenosine salvage pathway (Ujhazy et al., 1996). Ecto-5′-nucleotidase specifically acts in adenosine salvage pathways, converting AMP to adenosine which is more readily taken up by the cell and utilized as a precursor for ATP production. Therefore, ecto-5′-nucleotidase may be acting in certain MDR cell lines as a mechanism by which the cell circumvents the loss of ATP (due to up-regulated transport proteins which possibly form ATP transport channels) by creating higher levels of adenosine from which the cell can produce ATP. Correspondingly, 63% of MDR cell line variants tested expressed ecto-5′-nucleotidase. These observations suggested that a salvage mechanism for extracellular nucleotides may be another way by which certain MDR cells counterbalance their ATP losses from efflux induced by the over-expression of ABC transporters involved in MMR. Consistent with this hypothesis, inhibitors of ecto-5′-nucleotidase conferred sensitivity to certain drugs in MDR cell lines which over-express the ecto-5′-nucleotidase.

It is also interesting to note that yeast, which do not have an adenosine salvage pathway (Boyum and Guidotti, 1997), do contain a PGP-like gene called STS 1 (Bissinger and Kucher, 1994). Therefore, since the adenosine salvage pathway is unlikely to be involved in yeast multidrug resistance, other mechanisms are likely to exist.

Recent reports have confirmed the existence of ATP in the extracellular matrix (ECM) of both multicellular organisms and unicellular organisms (Sedaa et al., 1990; Boyum and Guidotti, 1997), respectively. However, no such reports are available which suggest the existence of ATP in the ECM of plants before the present invention. These reports have prompted further investigations of the fate of ATP outside the cell. One of the largest gradients in biological systems is that of ATP. It is a million-fold more concentrated inside the cell than outside. Phosphatases are enzymes with the ability to hydrolyze ATP and to a lesser extent, the beta phosphate of ADP (Plesner, 1995). Extracellular phosphatases are generally referred to as ectophosphatases A type of ectophosphatase is ecto-pyrase. Given reports that show the existence of extracellular ATP, one observation regarding ectoapyrase is that it hydrolyzes the extracellular ATP. In fact, work in animal systems has shown that apyrases hydrolyze ATP in the ECM as part of the adenosine salvage pathway conjointly with ecto-5′ ectonucleotidase (Che, 1992).

What has been lacking in the art are particular methods and compositions that allow modification of cellular ATP gradients to increase the uptake of cytotoxic agents by cells. It would be particularly useful to identify methods and compositions that result in increased uptake by cells of herbicides, antibiotics, fungicides, insecticides, chemotherapeutics and other active ingredients. This may result in increased efficacy in the use of such agents, as well as allow use of lower concentrations of such agents. This may provide cost, health and environmental benefits. This may also allow effective treatment of MDR cells, such as antibiotic-resistant bacteria or chemotherapy-resistant tumor cells.

### SUMMARY OF THE INVENTION

In one aspect, the invention provides a cytotoxic composition comprising an ectophosphatase inhibitor and a cytotoxic agent set forth in Table 1. In one embodiment of the invention, the cytotoxic agent is selectively cytotoxic. In other embodiments, the cytotoxic composition may be further defined as a herbicidal composition and the cytotoxic agent a herbicide, may be further defined as an insecticidal composition and the cytotoxic agent an insecticide, may be further defined as a fungicidal composition and the cytotoxic agent a fungicide and/or may be further defined as an antibiotic composition and the cytotoxic agent an antibiotic. In an antibiotic composition, the antibiotic may be from a class selected from the group consisting of Beta-lactam, Semisynthetic penicillin, Clavulanic Acid, Monobactams, Carboxypenems, Aminoglycosides, Glycopeptides, Lincomycins, Macrolides, Polyenes, Rifamycins, Tetracyclines, Semisynthetic, tetracycline and Chloramphenicol. In certain embodiments of the invention, the ectophosphatase inhibitor may be selected from the group consisting of the compounds of formulae I-XX.

In another aspect, the invention provides a plant growth regulator composition comprising an ectophosphatase inhibitor and a plant growth regulator agent set forth in Table 1. In one embodiment of the invention, the ectophosphatase inhibitor is selected from the group consisting of the compounds of formulae I-XX.

In yet another aspect, the invention provides a chemotherapeutic composition comprising an ectophosphatase inhibitory compound and a chemotherapeutic agent. In one embodiment of the invention, the chemotherapeutic agent is a chemotherapeutic agent set forth in Table 3. In further embodiments of the invention, the ectophosphatase inhibitor is selected from the group consisting of the compounds of formulae I-XX.

In still yet another aspect, the invention provides a method of killing or inhibiting the growth of a plant, comprising contacting said plant with an effective amount of a herbicidal composition provided by the invention. The method may be carried out with potentially any plant, including a monocotyledonous plant or a dicotyledonous plant.

In still yet another aspect, the invention provides a method of killing or inhibiting the growth of a tumor cell, comprising contacting said tumor cell with an effective amount of a chemotherapeutic composition of the invention. In the method, contacting may comprise administering the composition to a patient in need thereof, wherein the patient comprises the tumor cell.

In still yet another aspect, the invention provides a method of killing or inhibiting the growth of a target cell, comprising contacting said cell with a composition formulated therefore. In certain embodiments of the invention, the cell may be a fungal cell, bacterial cell, or insect cell.

In still yet another aspect, the invention provides a method of increasing the effectiveness of a cytotoxic agent, comprising admixing said cytotoxic agent with an ectophosphatase inhibitor, wherein the cytotoxic agent is selected from the group set forth in Table 1. In certain embodiments of the invention, the cytotoxic agent may be further defined as a herbicide, insecticide, fungicide, and an antibiotic. Examples of antibiotics include those from the following classes: Beta-lactam, Semisynthetic penicillin, Clavulanic Acid, Monobactams, Carboxypenems, Aminoglycosides, Glycopeptides, Lincomycins, Macrolides, Polyenes, Rifamycins, Tetracyclines, Semisynthetic, tetracycline and Chloramphenicol. In one embodiment of the invention. In one embodiment of the invention, a cytotoxic agent is a chemotherapeutic agent, for example, selected from the group set forth in Table 3.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C Expression of apyrase in pea and in transgenic plants (FIG. 1A) Immunoblot analysis of subcellular fractions from etiolated pea plants. (FIG. 1B) Top, the total phosphate accumulated in the shoots of three independent transgenic plants. Bottom, a corresponding Immunoblot performed on protein from ECM of wild-type and transgenic plants. (FIG. 1C) Assay of phosphatase activity in the ECM fraction of OE1 and wild-type.

FIGS. 2A-C Transport of the products of ATP hydrolysis by transgenic plants overexpressing apyrase and by wild-type plants.

FIGS. 3A-D Conference of resistance to cycloheximide (FIG. 3A, B) and nigencin (FIG. 3C, D) in wild-type and ectophosphatase deficient yeast over-expressing the Arabidopsis plant ABC transporter, AtPGP-1.

FIGS. 4A-B-3 Conference of resistance to cycloheximide (FIG. 4A) and cytokinin (FIG. 4B-1-FIG. 4B-3) in Arabidopsis plants over-expressing either the ectophosphatase, apyrase, or the ABC transporter, AtPGP-1.

FIGS. 5A-B Graph showing the growth turbidity of YMR4 yeast over-expressing the Arabidopsis plant ABC transporter AtPGP-1 grown in cycloheximide (FIG. 5A) or nigericin (FIG. 5B).

FIG. 6 Graph showing germination rate of Arabidopsis plants grown in the presence of cycloheximide which over-express either the ectophosphatase, apyrase, or the ABC transporter AtPGP-1.

FIG. 7 Graph of steady-state levels of ATP in the extracellular fluid of wildtype yeast cells grown in the presence or absence of glucose and in the presence or absence of over-expression of the Arabidopsis plant ABC transporter, AtPGP-1.

FIG. 8 Graph showing that over-expression of Arabidopsis plant ABC transporter, AtPGP-1, in yeast can double the steady-state levels of ATP in the extracellular fluid.

FIG. 9 Graph showing that a yeast mutant, YMR4, that has a deficient ectophosphatase, accumulates ATP in the extracellular fluid and the over-expression of AtPGP-1 increases the accumulation of ATP.

FIG. 10 Graph showing results of a pulse-chase experiment in either wildtype yeast cells or a yeast mutant, YMR4, which is deficient in ectophosphatase activity, in the presence and absence of over-expression of Arabidopsis plant ABC transporter, AtPGP-1, demonstrating an early differential ATP efflux of cells over-expressing AtPGP-1.

FIG. 11 Graph of ATP levels on the surface of leaves of Arabidopsis plants over-expressing AtPGP-1 (MDR 1).

FIG. 12 Effects of phosphatase inhibitor in wild-type and AtPGP-1 (MDRI) overexpressing Arabidopsis plants.

FIG. 13 Growth effects of cycloheximide and extracellular ATP on wildtype and MDR1 overexpressing S. cerevisiae yeast cells which have either never seen cycloheximide or which have been previously selected in cycloheximide.

FIG. 14 Growth effects of cycloheximide, adenosine and phosphate on wildtype and AtPGP-1 overexpressing S. cerevisiae yeast cells.

### DETAILED DESCRIPTION OF THE INVENTION

The present invention provides methods and compositions for modulating the sensitivity of cells to cytotoxic compounds and other active agents. In accordance with the invention, such modulation may be carried out by modifying cellular ATP gradients, for example, by contacting a plant, animal, yeast or bacterial cell with an ectophosphatase inhibitor contemporaneously with a cytotoxic agent or other active ingredient, or combinations thereof. As used herein, the term “ectophosphatase inhibitor” refers to a compound that has the ability to specifically inhibit ectophosphatase activity. A “cytotoxic agent” is a compound capable of selectively or non-selectively killing or inhibiting the growth of a cell, including antibiotics, fungicides, herbicides, insecticides and chemotherapeutic agents.

The efficacy of one or more cytotoxic agents may be increased in accordance with the invention by reducing the ATP gradient across biological membranes, which is effectuated through the modulation of an ectophosphatase either alone or together with an ABC transporter molecule. Modulation of resistance to a cytotoxic compound as described herein is useful, for example, in increasing the sensitivity of plants to herbicides; reducing drug resistance in tumor cells for improved chemotherapy applications; and reducing resistance to antibiotics, antifungal agents, insecticides and other cytotoxic agents for the treatment of infections and disease. Provided by the invention are specific combinations of ectophosphatase inhibitors and cytotoxic compounds or other active ingredients that may be used in these applications.

In accordance with certain embodiments of the invention, the manipulation of extracellular ATP levels to alter ATP gradient across biological membranes in cells by inhibition of ectophosphatases results in diminishing or removing the ATP gradient that may otherwise prevent access of cytotoxic agents to the cell. In some instances, access may be minimized by efflux of cytotoxic agents from the cell. This efflux is likely effectuated through the “piggy-back” efflux of drug molecules with ATP, a phenomenon known as symport.

In accordance with the invention, inhibitors of ectophosphatases may be administered contemporaneously with cytotoxic agents, including herbicides, antibiotics, fungicides, insecticides and other active ingredients, as well as chemotherapeutics. By “contemporaneously,” it is meant within sufficient temporal proximity that the increased sensitivity of a cell to the relevant cytotoxic agent may be achieved in combination with contacting the cell with the agent. In certain embodiments of the invention, ectophosphatase inhibitors are used as adjuvants with cytotoxic agents, that is, are admixed with one or more cytotoxic agents or other active ingredients.

It is therefore an, object of the present invention to provide inhibitors of ectophosphatases in physiological compositions for modulating cell growth and/or survival, including, but not limited to, MDR cells. Such physiological compositions may comprise a small molecule capable of inhibiting an ectophosphatase and a physiologically acceptable carrier or diluent together with a therapeutic agent. As used herein, the term “physiologically acceptable carrier or diluent” means any and all solvents, dispersion media, antibacterial and antifungal agents, microcapsules, liposomes, cationic lipid carriers, isotonic and absorption delaying agents and the like which are not incompatible with the ectophosphatase inhibitors. The use of such media and agents for physiologically active substances is well known in the art. Supplementary active ingredients may also be incorporated into the compositions.

In certain embodiments, only an endogenous ectophosphatase is inhibited. In particular embodiments of the invention, the ectophosphatase is human apyrase. In other embodiments, the ectophosphatase is a plant, bacterial, fungal or yeast ectophosphatase.

The inhibition of ectophosphatases is useful in increasing sensitivity to cytotoxic agents and/or reduction of drug resistance in cells. In one embodiment of the invention, the inhibition of ectophosphatases results in a loss of resistance to a cytotoxic agent. In another embodiment of the invention, administration of such inhibitory molecules is in conjunction with the administration of chemotherapeutic agents in tumor cells.

Using high throughput screens, ectophosphatase inhibitors may be isolated, for example, by screening a small molecule library (e.g. a combinatorial library) for inhibitory activity to ectophosphatase (e.g. apyrase activity). Once ectophosphatase inhibitory molecules are isolated from such a screen, the inhibitors may be further tested for their ability to specifically inhibit the ATPase activity of the ectophosphatase.

Exemplary ectophosphatase inhibitory molecules for use with the current invention are preferably chemically stable and physiologically active including, but not limited to, the compounds of formulae I-XX.

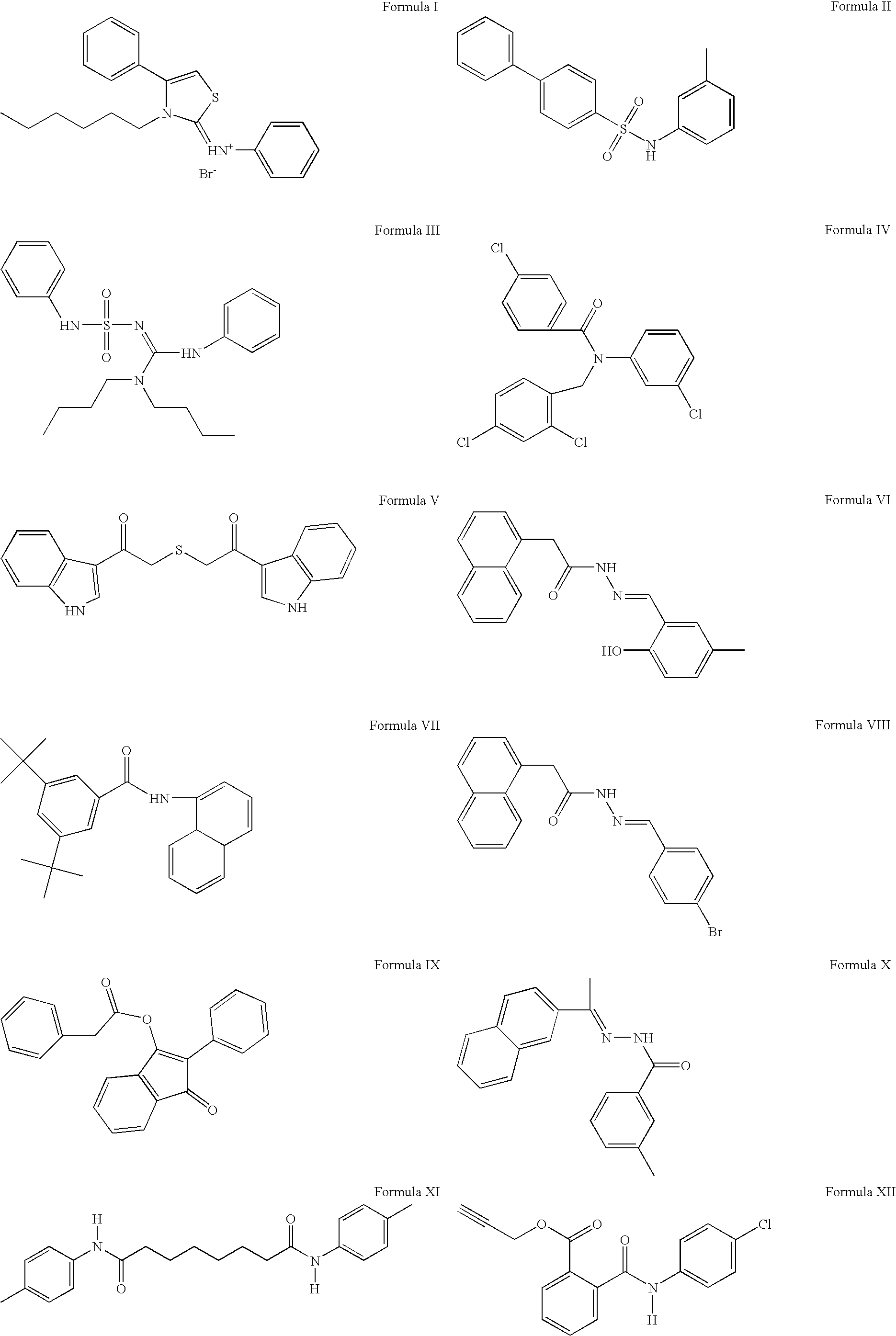

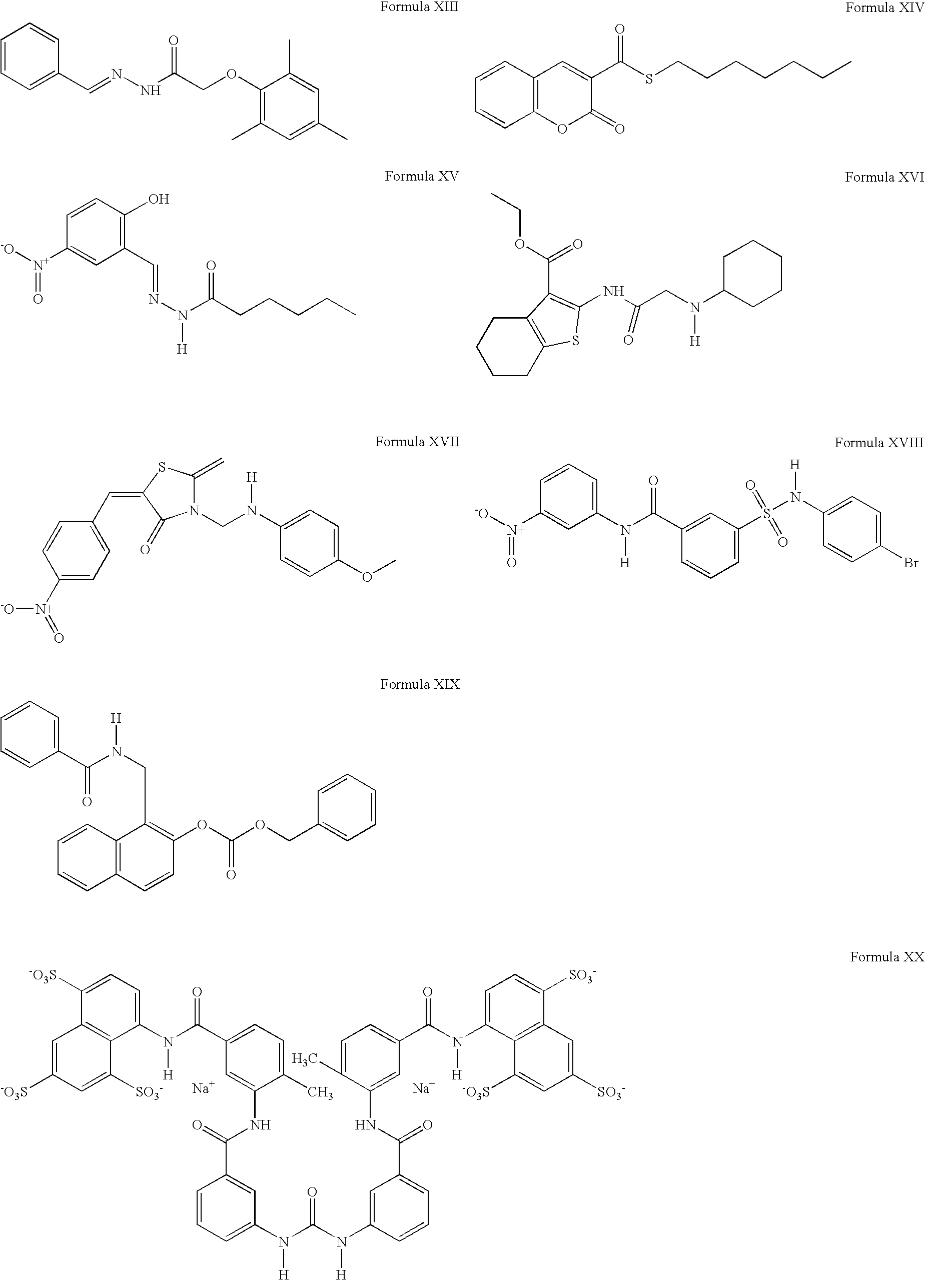

Preliminary pharmacophore studies revealed that the small molecules represented by Formulae I through XX fall into five classes of compounds (sulfanamides, guanidines, aminothiazoles, thioketones and benzamides). Most of these chemical classes are found in other physiologically-active compounds, including those having pharmaceutical and therapeutic use. For example, sulfanimides are widely used as antibiotics. Additionally, studies for the isolation of small molecules capable of reversing MDR have described molecules belonging to two of the classes of molecules of the present invention (Medina et al. 1998; Dhamant et al., 1992). The molecules described by Medina et al. have been shown to affect MDR and the mode of action of the molecules is believed to involve tubulin interactions. The thiazine derivatives described by Dhamant et al. reverse the resistance in tumor cells to vincristine.

The ectophosphatase inhibitory molecules are useful in increasing the activities of various agents by increasing the availability of the agents to cells. In certain embodiments of the invention, this may comprise reversing multidrug resistance (MDR) in an organism. In other embodiments, this may be achieved with a cell that does not exhibit MDR. Such cells may or may not comprise an up-regulated ectophosphatase

MDR reversal and/or increasing of sensitivity to a cytotoxic agent in cells may be shown by growing the cells in the presence of relevant drugs and in the presence and absence of the inhibitor. Cells which cannot grow in the presence of a drug in the presence of an ectophosphatase inhibitor, have a reversal in MDR and/or increase in sensitivity to the agent.

The ectophosphatase inhibitory compositions of the present invention are useful in reversing drug resistance in mammalian cell lines grown in the presence of a drug (e.g. a chemotherapeutic agent). Analysis of sensitivity to a chemotherapeutic composition in mammalian cells may be shown, for example, by using the fluorescent compound calcein-AM. Esterases present in cells cleave the aceto-methoxy ester (AM) from the calcein-AM and liberate calcein. Calcein is a fluorescent compound which is excitable by the 488 nm laser of a FACS Caliber flow cytometer (Becton Dickenson, Franklin Lakes, N.J.), while the uncleaved calcein-AM is not excitable. Wild type cells incubated in the presence of calcein-AM show a high level of fluorescence while MDR state cells, which efflux the calcein-AM faster than the cellular esterases can cleave it, do not show a high level of fluorescence. The mammalian cells can be tested for the reversal of MDR with ectophosphatase inhibitors by the amount of calcein fluorescence detected in the cells.

Specificity of ectophosphatase inhibitors may be tested with the screening assays described herein. Inhibitors can be tested for their ability to inhibit acid phosphatases, alkaline phosphatases, myosin phosphatases and the luciferase ATPase. The assays may be performed using techniques known in the art.

In one embodiment of the invention, the ectophosphatase is an apyrase and the ectophosphatase inhibitor is a molecule selected from among molecules represented by the Formulae I through XX. In another embodiment, the ectophosphatase is apyrase and the ectophosphatase inhibitor is a molecule selected from among molecules represented by the Formulae I through V.

In a further embodiment of the invention, the ectophosphatase is apyrase and the ectophosphatase inhibitor is a molecule represented by Formula XX. Formula XX is suramin or 8,8′[Carbonylbis[imino-3,1-phenylenecarbonylimino(4-methyl-3,1-phenylene)carbonyl imino]]bis-1,3,5-naphthalenetrisulfonic acid. Suramin has been reported as a potent non-competitive inhibitor of ectoapyrase activity associated with the plasma membrane of cholinergic nerve terminal of Torpedo mannorata electric organs (arti et al., 1996).

It is contemplated that in certain embodiments of the invention an ectophosphatase inhibitor may inhibit an ectophosphatase active on the outer membrane of an organelle of a eukaryotic cell. In this manner, contacting the cell with the ectophosphatase inhibitor may be used to decrease the ATP gradient across the organelle and thereby facilitate uptake of a biologically active agent by the organelle.

### I. Formulations

Ectophosphatase inhibitor compositions, which may be acidic or basic in nature, can form a wide variety of salts with various inorganic and organic bases or acids, respectively. These salts may be physiologically acceptable for in vivo administration in mammals, including humans, as well as formulations for ex vivo applications, including agricultural use. Salts of acidic compounds are readily prepared by treating the acidic compound with an appropriate molar quantity of the chosen inorganic or organic base in an aqueous or suitable organic solvent and then evaporating the solvent to obtain the salt. Salts of basic compounds can be obtained similarly by treatment with the desired inorganic or organic acid and subsequent solvent evaporation and isolation. The skilled artisan can produce salts of ectophosphatase inhibitors using techniques known in the art.

The skilled artisan readily can determine the amount of the ectophosphatase inhibitor that is required to inhibit ectophosphatase by measuring ATPase activity in the presence and absence of varying amounts of the inhibitor. Phosphatase activity can be determined by assessing the dephosphorylation of ATP and liberation of phosphate as described herein. Additionally, parameters may be measured that are known to be associated with ectophosphatase activity to determine whether the molecule has ectophosphatase inhibitory activity. For example, ectophosphatase inhibitory activity may be measured in cells (e.g., yeast, plant, bacterial, mammalian, and tumor cell lines) by assessing the loss of resistance to drugs. Furthermore, the ectophosphatase inhibitory molecules of the present invention may be tested for specific inhibitory activity to ectophosphatases versus general phosphatases or for specific inhibitory activity for a particular ectophosphatase activity (e.g., apyrase).

The present invention also provides physiologically acceptable compositions comprising an ectophosphatase inhibitor, a cytotoxic agent or other active ingredient and a physiologically acceptable carrier or diluent. In certain embodiments of the invention, as set forth in detail below, compositions are provided comprising one or more ectophosphatase inhibitor and one or more plant growth regulator, herbicide, fungicide, insecticide, antibiotic, chemotherapeutic agent or other active compound. In a further embodiment of the invention, the ectophosphatase is selected from the compounds of formulae I-XX in the appropriate carriers, diluents or other active ingredients.

The use of physiologically acceptable carriers or diluents is well known in the art. The techniques of preparation of pharmaceutical compositions are generally well known in the art as exemplified by Remington's Pharmaceutical Sciences, 16th Ed. Mack Publishing Company, 1980, which reference is specifically incorporated herein by reference in its entirety. Formulations of the present invention may be stable under the conditions of manufacture and storage and must be preserved against contamination by microorganisms.

The physiological forms of the compositions of the invention suitable for administration include sterile aqueous solutions or dispersions and sterile powders for the extemporaneous preparation of sterile injectable solutions or dispersions. Typical carriers include a solvent or dispersion medium containing, for example, water buffered aqueous solutions (i.e., biocompatible buffers), ethanol, polyols such as glycerol, propylene glycol, polyethylene glycol, suitable mixtures thereof, surfactants, and vegetable oils. Isotonic agents such as sugars or sodium chloride may be incorporated into the subject compositions.

Pharmaceutical compositions in accordance with the invention may be used by themselves or in combination with other forms active ingredients or therapeutics. One embodiment of the invention provides formulations for parenteral administration, e.g., formulated for injection via the intravenous, intramuscular, sub-cutaneous or other such routes. Typically, such compositions can be prepared as injectables, either as liquid solutions or suspensions; solid forms suitable for using to prepare solutions or suspensions upon the addition of a liquid prior to injection also can be prepared; and the preparations also can be emulsified.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Carriers used also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Other modes of administration can also find use with the invention. For instance, pharmaceutical compounds may be formulated in suppositories and, in some cases, aerosol and intranasal compositions. For suppositories, the vehicle composition will include traditional binders and carriers such as polyalkylene glycols or triglycerides. Such suppositories may be formed from mixtures containing the active ingredient in the range of about 0.5% to about 10% (w/w), preferably about 1% to about 2%.

Ectophosphatase inhibitors will commonly comprise from less than 1% to about 10% w/v of a composition with an active ingredient, including about 1%, 3%, 5%, &% and about 10%. Greater or lesser amounts of the ectophosphatase inhibitors may similarly be used. Desired concentrations may readily be determined by serial preparation and assay of compositions comprising varying amounts of the selected ectophosphatase inhibitor compound. For example, in the case of herbicidal compositions, a series of test compositions comprising from about 0.1% to about 25% w/v of an ectophosphatase inhibitor together with a test herbicide may be analyzed by leaf painting and comparison of the results. Safety, efficacy, cost and other concerns may each be taken into account in selecting the desired concentrations. Alternatively, pharmaceutical compositions may be analyzed using in vitro or in vivo models.

Oral compositions may be prepared in the form of solutions, suspensions, tablets, pills, capsules, sustained release formulations, or powders. These compositions can be administered, for example, by swallowing or inhaling. Where a pharmaceutical composition is to be inhaled, the composition will preferably comprise an aerosol. Exemplary procedures for the preparation of aqueous aerosols may be found in U.S. Pat. No. 5,049,388, the disclosure of which is specifically incorporated herein by reference in its entirety. Preparation of dry aerosol preparations are described in, for example, U.S. Pat. No. 5,607,915, the disclosure of which is specifically incorporated herein by reference in its entirety.

Also useful is the administration of the compounds described herein directly in transdermal formulations with permeation enhancers such as DMSO. These compositions can similarly include any other suitable carriers, excipients or diluents. Other topical formulations can be administered to treat certain disease indications. For example, intranasal formulations may be prepared which include vehicles that neither cause irritation to the nasal mucosa nor significantly disturb ciliary function. Diluents such as water, aqueous saline or other known substances can be employed with the subject invention. The nasal formulations also may contain preservatives such as, but not limited to, chlorobutanol and benzalkonium chloride. A surfactant may be present to enhance absorption of the subject compounds by the nasal mucosa.

Upon formulation, solutions may be administered in a manner compatible with the dosage formulation and in such amount as is therapeutically effective. The formulation of choice can be accomplished using a variety of excipients including, for example, pharmaceutical grades of mannitol, lactose, starch, magnesium stearate, sodium saccharin cellulose, magnesium carbonate, and the like. Typically, pharmaceutical compositions will contain from less than 1% to about 95% of the active ingredient, preferably about 10% to about 50%. Preferably, between about 10 mg/kg patient body weight per day and about 25 mg/kg patient body weight per day will be administered to a patient, including a human patient. The frequency of administration will be determined by the care given based on patient responsiveness. Other effective dosages can be readily determined by one of ordinary skill in the art through routine trials establishing dose response curves.

Regardless of the mode of administration, suitable pharmaceutical compositions in accordance with the invention will generally include an amount of active ingredient admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, which reference is specifically incorporated herein by reference in its entirety. It should be appreciated that endotoxin contamination should be kept minimally at a safe level, for example, less that 0.5 ng/mg protein. Moreover, for human administration, preparations should meet sterility, pyrogenicity, general safety and purity standards as required by FDA Office of Biological Standards.

The therapeutically effective doses are readily determinable using an animal model. For example, experimental animals exhibiting a target infection or other ailment are frequently used to optimize appropriate therapeutic doses prior to translating to a clinical environment. Such models are known to be very reliable in predicting effective therapies. In certain embodiments, it may be desirable to provide a continuous supply of therapeutic compositions to the patient. For intravenous or intraarterial routes, this is accomplished by drip system. For topical applications, repeated application would be employed. For various approaches, delayed release formulations could be used that provided limited but constant amounts of the therapeutic agent over and extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the injections are administered. It is believed that higher doses may be achieved via perfusion, however.

Therapeutic kits comprising the compositions described herein are also provided by the invention. Such kits will generally contain, in suitable container means, a pharmaceutically or agriculturally acceptable formulation of the active ingredient. The kits also may contain other pharmaceutically acceptable formulations, such as an antiinfective agent, including an insecticide, antifungal or antibacterial, as well as a herbicide.

The kits may have a single container means that contains the active ingredient, with or without any additional components, or they may have distinct container means for each desired agent. When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent also may be provided in another container means. The container means of the kit will generally include at least one vial, test tube, flask, bottle, syringe or other container means, into which the active compound, and any other desired agent, may be placed and, preferably, suitably aliquoted. Where additional components are included, the kit will also generally contain a second vial or other container into which these are placed, enabling the administration of separated designed doses. The kits also may comprise a second/third container means for containing a sterile, pharmaceutically acceptable buffer or other diluent.

The kits also may contain a means by which to administer the compositions to an animal or patient, e.g., one or more needles or syringes, or even an eye dropper, pipette, or other such like apparatus, from which the formulation may be injected into the animal or applied to a afflicted area of the body. The kits of the present invention will also typically include a means for containing the vials, or such like, and other component, in close confinement for commercial sale, such as, e.g., injection or blow-molded plastic containers into which the desired vials and other apparatus are placed and retained.

The invention also provides compositions formulated for agricultural use. Examples of such formulations include herbicidal, fungicidal and plant growth regulator compositions. Specific formulations for plant application are known to those of skill in the art and are described, for example, in U.S. Pat. No. 6,242,382, the disclosure of which is specifically incorporated herein by reference in its entirety.

Examples of ingredients that may be included in a composition of the invention formulated for application to plants include surfactants, solid or liquid carriers, solvents and binders. Examples of suitable surfactants that may be used for application to plants include the alkali metal, alkaline earth metal or ammonium salts of aromatic sulfonic acids, e.g., ligno-, phenol-, naphthalene- and dibutylnaphthalenesulfonic acid, and of fatty acids of arylsulfonates, of alkyl ethers, of lauryl ethers, of fatty alcohol sulfates and of fatty alcohol glycol ether sulfates, condensates of sulfonated naphthalene and its derivatives with formaldehyde, condensates of naphthalene or of the naphthalenesulfonic acids with phenol and formaldehyde, condensates of phenol or phenolsulfonic acid with formaldehyde, condensates of phenol with formaldehyde and sodium sulfite, polyoxyethylene octylphenyl ether, ethoxylated isooctyl-, octyl- or nonylphenol, tributylphenyl polyglycol ether, alkylaryl polyether alcohols, isotridecyl alcohol, ethoxylated castor oil, ethoxylated triarylphenols, salts of phosphated triarylphenolethoxylates, lauryl alcohol polyglycol ether acetate, sorbitol esters, lignin-sulfite waste liquors or methylcellulose, or mixtures of these. Common practice in the case of surfactant use is to include about 0.5 to 25% by weight, based on the total weight of the solid mixture.

Compositions for application to plants may be solid or liquid. Where solid compositions are used, it may be desired to include one or more carrier materials with the active compound. Examples of carriers include mineral earths such as silicas, silica gels, silicates, talc, kaolin, attaclay, limestone, chalk, loess, clay, dolomite, diatomaceous earth, calcium sulfate, magnesium sulfate, magnesium oxide, ground synthetic materials, fertilizers such as ammonium sulfate, ammonium phosphate, ammonium nitrate, thiourea and urea, products of vegetable origin such as cereal meals, tree bark meal, wood meal and nutshell meal, cellulose powders, attapulgites, montmorillonites, mica, vermiculites, synthetic silicas and synthetic calcium silicates, or mixtures of these.

For liquid solutions, water-soluble compounds or salts may be included, such as sodium sulfate, potassium sulfate, sodium chloride, potassium chloride, sodium acetate, ammonium hydrogen sulfate, ammonium chloride, ammonium acetate, ammonium formate, ammonium oxalate, ammonium carbonate, ammonium hydrogen carbonate, ammonium thiosulfate, ammonium hydrogen diphosphate, ammonium dihydrogen monophosphate, ammonium sodium hydrogen phosphate, ammonium thiocyanate, ammonium sulfamate or ammonium carbamate.

Other exemplary components in compositions of the invention include binders such as polyvinylpyrrolidone, polyvinyl alcohol, partially hydrolyzed polyvinyl acetate, carboxymethylcellulose, starch, vinylpyrrolidone/vinyl acetate copolymers and polyvinyl acetate, or mixtures of these; lubricants such as magnesium stearate, sodium stearate, talc or polyethylene glycol, or mixtures of these; antifoams such as silicone emulsions, long-chain alcohols, phosphoric esters, acetylene diols, fatty acids or organofluorine compounds, and complexing agents such as: salts of ethylenediaminetetraacetic acid (EDTA), salts of trinitrilotriacetic acid or salts of polyphosphoric acids, or mixtures of these.

In certain embodiments of the invention, compositions are provided with herbicidal activity which comprise one or more ectophosphatase inhibitor(s) and a herbicide selected from the group consisting of pendimethalin, dicamba, 2,4-D, glufosinate, bentazon, trifluralin, oryzalin, S-metolachlor, nicosulfuron, MSMA, Epic™ and Balance™. In one embodiment of the invention, the ectophosphatase inhibitor is selected from the group of the compounds of formulae I-XX. In certain other embodiments of the invention, compositions with fungicidal activity are provided which comprise one or more ectophosphatase inhibitor(s) in combination with metalaxyl, tebuconazole, propiconazole, chlorothalonil and/or fluconazole.

Non-limiting examples of combinations of cytotoxic agents and ectophosphatase inhibitors for use in accordance with the invention include: fluconazole with compound(s) of formula II, IX, XIV, XVI, XV, X and/or XII; formula I and/or II with pendimethalin, dicamba, 2,4-D, bentazon, trifluralin, flufenacet/isoxaflutole or isoxaflutole; glufosinate with formula II; oryzalin with formula I; nicosulflron with formula II; MSMA with formula XVIII; S-metolachlor with formula VI and/or X; metribuzin with formula I, II and/or X; diuron with formula VI; metalaxyl with formula X; chlorothalonil with formula II, X, IX, XI, XVI; propiconazole with formula I, X, XIV and/or XVI; mancozeb with formula I, VI and/or X; captan with formula XIV and/or XVI; tebuconazole with formula I and/or X; imidacloprid with formula II, X, XVIII, XVI and/or XV; and fertilizers such as Nitrogen, Phosphate, Potash—12:12:12 with formula I and/or II.

Also envisioned for use with the invention are compositions comprising combinations of ectophosphatase inhibitors and cytotoxic agents or other active ingredients, including a herbicide, insecticide, antibiotic, fungicide, plant growth regulator and/or hormone. Such combinations may provide enhanced activity of the active ingredient relative to the use of a single ectophosphatase inhibitor. Non-limiting examples of such combinations of ectophosphatase inhibitors include combinations of two, three, four or more ectophosphatase inhibitors selected from formulas I, II, VI, X, XV, XIV, XVI, XVI, XV, and XVIII. Included for illustrative purposes are the following combinations: I and II; I, II and X; XIV and XVI; XVIII and XVI; XVI, XVI and XV; VI and X; II and X; and I and XVIII.

Specifically contemplated by the inventors are compositions comprising an ectophosphatase inhibitor and an active ingredient set forth in Table 1.

TABLE 1
Cytotoxic agents and other active ingredients

| | | U.S. EPA PC | CA Chem | | |
|---|---|---|---|---|---|
| Chemical Name | CAS Number | Code | Code | Use Type | Chemical Class |
| Bis(2-ethylhexyl) phthalate | 117-81-7 | 3E+05 | NA 11 | N/A | Alkyl Phthalate |
| Butyl benzyl phthalate | 85-68-7 | NA 141 | 3080 | N/A | Alkyl Phthalate |
| Chlorthal-dimethyl | 1861-32-1 | 78701 | 179 | Herbicide | Alkyl Phthalate |
| Di-n-octyl phthalate | 117-84-0 | NA 303 | 223 | N/A | Alkyl Phthalate |
| Dibutyl phthalate | 84-74-2 | 28001 | 199 | Insect | Alkyl Phthalate |
| | | | | Repellent | |
| Diethyl phthalate | 84-66-2 | 1E+05 | 3326 | N/A | Alkyl Phthalate |
| Diisodecyl phthalate | NA 1037 | NA 132 | 2513 | N/A | Alkyl Phthalate |
| Dimethyl phthalate | 131-11-3 | 28002 | 232 | Insect | Alkyl Phthalate |
| | | | | Repellent | |
| Dioctyl terephthalate | NA 724 | NA 133 | 2528 | N/A | Alkyl Phthalate |
| Diphenyl phthalate | 84-62-8 | 17001 | NA 147 | N/A | Alkyl Phthalate |
| Isooctyl phthalate | 27554-26-3 | NA 483 | 2262 | N/A | Alkyl Phthalate |
| n-Butyl phthalate | 84-74-2 | 28001 | 3081 | Insecticide | Alkyl Phthalate |
| Poly glyceryl phthalate ester of | NA 327 | NA 608 | 3331 | Adjuvant, | Alkyl Phthalate |
| coconut oil fatty acid | | | | Soap/Surfactant | |
| 1,3-Dimethyl-1,1,3,3- | 22232-20-8 | 34601 | NA 303 | N/A | Bis-Carbamate |
| disiloxanetetrol-1,3- | | | | | |
| bis(dimethylthiocarbamate) | | | | | |
| 2,3(and 3,4)-Dichlorobenzyl | 62046-37-1 | 3E+05 | NA 136 | Insecticide | N-Methyl Carbamate |
| methylcarbamates | | | | | |
| 2,4-Dimethyl-1,3-dithiolane-2- | 26419-73-8 | 4E+05 | NA 143 | Insecticide | N-Methyl Carbamate |
| carboxaldehyde O- | | | | | |
| (methylcarbamoyl)oxime | | | | | |
| 2-(4,5-Dimethyl-1,3-dioxolan- | ######## | 4E+05 | NA 145 | Insecticide | N-Methyl Carbamate |
| 2-yl)phenyl-N- | | | | | |
| methylcarbamate | | | | | |
| 2-chloro-4,5-xylyl N-hydroxy- | 14357-82-5 | 2E+05 | NA 117 | Insecticide | N-Methyl Carbamate |
| N-methylcarbamate | | | | | |
| 2-cyclopentylphenyl | 3282-00-6 | 3E+05 | NA 132 | Insecticide | N-Methyl Carbamate |
| methylcarbamate | | | | | |
| 2-Hydroxyphenyl | 10309-97-4 | 2E+05 | NA 118 | Insecticide | N-Methyl Carbamate |
| methylcarbamate | | | | | |
| 3,4-Dichlorobenzyl | 2328-31-6 | 3E+05 | NA 136 | Insecticide | N-Methyl Carbamate |
| methylcarbamate | | | | | |
| 3,5-Diisopropylphenyl | 330-64-3 | 4E+05 | NA 142 | Insecticide | N-Methyl Carbamate |
| methylcarbamate | | | | | |
| 3-hydroxycarbofuran | 16655-82-6 | 2E+05 | 4074 | Breakdown | N-Methyl Carbamate |
| | | | | product | |
| 3-iodo-2-propynyl butyl | 55406-53-6 | 1E+05 | 1938 | Fungicide, | Other Carbamate |
| carbamate | | | | Wood | |
| | | | | Preservative | |
| 3-ketocarbofuran | 16709-30-1 | NA 176 | NA 164 | Breakdown | N-Methyl Carbamate |
| | | | | product | |
| 4- | 17702-57-7 | 4E+05 | NA 142 | Insecticide | N-Methyl Carbamate |
| (((Dimethylamino)methylene)amino)- | | | | | |
| m-tolyl | | | | | |
| methylcarbamate | | | | | |
| 4-(Methylamino)-3,5-xylyl | 10389-50-1 | 2E+05 | NA 118 | Insecticide | N-Methyl Carbamate |
| methylcarbamate | | | | | |
| 4-(Methylformamido)-3,5- | 10233-94-0 | 2E+05 | NA 118 | Insecticide | N-Methyl Carbamate |
| xylyl methylcarbamate | | | | | |
| 4-(Methylthio)-m-tolyl | 3566-00-5 | 6E+05 | NA 157 | Insecticide | N-Methyl Carbamate |
| methylcarbamate | | | | | |
| 4-Amino-3,5-xylyl | 831-76-5 | 2E+05 | NA 117 | Insecticide | N-Methyl Carbamate |
| methylcarbamate | | | | | |
| 5,6,7,8-Tetrahydro-1-naphthyl | 1136-84-1 | 4E+05 | NA 148 | Insecticide | N-Methyl Carbamate |
| methylcarbamate | | | | | |
| 6-(and 2)-chloro-3,4-xylyl | 8063-85-2 | 38701 | NA 322 | Insecticide | N-Methyl Carbamate |
| methylcarbamate | | | | | |
| 6-Methyl-2-propyl-4- | 2532-49-2 | 6E+05 | NA 157 | N/A | Other Carbamate |
| pyrimidinyl dimethylcarbamate | | | | | |
| Alanycarb | 83130-01-2 | NA 175 | NA 47 | Insecticide | Other Carbamate |
| Aldicarb | 116-06-3 | 98301 | 575 | Insecticide, | N-Methyl Carbamate |
| | | | | Nematicide | |
| Aldicarb sulfoxide | 1646-87-3 | 1E+05 | 2361 | Breakdown | N-Methyl Carbamate |
| | | | | product | |
| Aldoxycarb | 1646-88-4 | 1E+05 | 2265 | Breakdown | N-Methyl Carbamate |
| | | | | product | |
| Allyxycarb | 6392-46-7 | 3E+05 | NA 133 | Insecticide | N-Methyl Carbamate |
| Aminocarb | 2032-59-9 | 44401 | 2435 | Insecticide | N-Methyl Carbamate |
| Asulam | 3337-71-1 | 1E+05 | 5076 | Herbicide | Other Carbamate |
| Asulam, sodium salt | 2302-17-2 | 1E+05 | 1746 | Herbicide | Other Carbamate |
| Bendiocarb | 22781-23-3 | 1E+05 | 1924 | Insecticide | N-Methyl Carbamate |
| Benfuracarb | 82560-54-1 | 1E+05 | NA 48 | Insecticide | Other Carbamate |
| Bufencarb | 2282-34-0 | 59302 | 91 | Insecticide | N-Methyl Carbamate |
| Bufencarb | 8065-36-9 | 59303 | NA 485 | Insecticide | N-Methyl Carbamate |
| Bufencarb, component of (with | 672-04-8 | 59301 | NA 484 | Insecticide | N-Methyl Carbamate |
| 059302) | | | | | |
| Butacarb | 2655-19-8 | 3E+05 | NA 135 | Insecticide | N-Methyl Carbamate |
| Butocarboxim | 34681-10-2 | NA 175 | NA 49 | Insecticide | N-Methyl Carbamate |
| Butoxycarboxim | 34681-23-7 | 1E+05 | 2201 | Insecticide | N-Methyl Carbamate |
| Butylate | 2008-41-5 | 41405 | 565 | Herbicide | Thiocarbamate |
| Calcium | 5895-18-1 | 14501 | NA 139 | Microbiocide | Dithiocarbamate |
| ethylenebis(dithiocarbamate) | | | | | |
| Carbanolate | 671-04-5 | 2E+05 | NA 124 | Insecticide | N-Methyl Carbamate |
| Carbaryl | 63-25-2 | 56801 | 105 | Insecticide, | N-Methyl Carbamate |
| | | | | Plant Growth | |
| | | | | Regulator, | |
| | | | | Nematicide | |
| Carbofuran | 1563-66-2 | 90601 | 106 | Insecticide, | N-Methyl Carbamate |
| | | | | Nematicide | |
| Carbosulfan | 55285-14-8 | 90602 | 2182 | Insecticide | N-Methyl Carbamate |
| Chlorbufam | 1967-16-4 | 6E+05 | NA 157 | Herbicide | Other Carbamate |
| Chlorprocarb | 23121-99-53 | 3E+05 | NA 136 | Herbicide | Other Carbamate |
| Chlorpropham | 101-21-3 | 18301 | 141 | Herbicide, | Other Carbamate |
| | | | | Plant Growth | |
| | | | | Regulator | |
| Cloethocarb | 51487-69-5 | 1E+05 | NA 100 | Insecticide, | N-Methyl Carbamate |
| | | | | Molluscicide | |
| Cufraneb | 11096-18-7 | NA 181 | NA 180 | Fungicide | Dithiocarbamate |
| Cycloate | 1134-23-2 | 41301 | 516 | Herbicide | Thiocarbamate |
| Desmedipham | 13684-56-5 | 1E+05 | 1748 | Herbicide | Bis-Carbamate |
| Diammonium | ######## | 14502 | NA 140 | Microbiocide | Dithiocarbamate |
| ethylenebis(dithiocarbamate) | | | | | |
| Dichlormate | 1966-58-1 | 3E+05 | 690 | Herbicide | N-Methyl Carbamate |
| Diethofencarb | 87130-20-9 | NA 184 | NA 183 | Fungicide | Other Carbamate |
| Dimepiperate | 61432-55-1 | NA 186 | NA 185 | Herbicide | Thiocarbamate |
| Dimetan | 122-15-6 | 1E+05 | NA 112 | Insecticide | Other Carbamate |
| Dimethyl (4-methyl-1,3- | 51543-98-7 | 1E+05 | NA 888 | Fungicide | Bis-Carbamate |
| phenylenebis(iminocarbonyl- | | | | | |
| 1H-benzimidazole-1,2- | | | | | |
| diyl))biscarbamate | | | | | |
| Dimetilan | 644-64-4 | 90101 | NA 844 | Insecticide | Other Carbamate |
| Dioxacarb | 6988-21-2 | 4E+05 | 4067 | Insecticide | N-Methyl Carbamate |
| EPTC | 759-94-4 | 41401 | 264 | Herbicide | Thiocarbamate |
| Esprocarb | 85785-20-2 | NA 203 | NA 202 | Herbicide | Thiocarbamate |
| Ethiofencarb | 29973-13-5 | 1E+05 | NA 50 | Insecticide | N-Methyl Carbamate |
| Ethiolate | 2941-55-1 | 1E+05 | 1793 | Herbicide | Thiocarbamate |
| Fenethacarb | 30087-47-9 | 3E+05 | NA 141 | Insecticide | N-Methyl Carbamate |
| Fenobucarb | 3766-81-2 | NA 176 | NA 51 | Insecticide | N-Methyl Carbamate |
| Fenothiocarb | 62850-32-2 | NA 188 | NA 187 | Insecticide | Thiocarbamate |
| Fenoxycarb | 79127-80-3 | 1E+05 | 2283 | Insecticide, | Other Carbamate |
| | | | | Insect Growth | |
| | | | | Regulator | |
| Fenoxycarb | 72490-01-8 | NA 228 | NA 228 | Insecticide, | Other Carbamate |
| | | | | Insect Growth | |
| | | | | Regulator | |
| Ferbam | 14484-64-1 | 34801 | 288 | Fungicide | Dithiocarbamate |
| Ferric nitroso dimethyl | 83542-83-0 | 5E+05 | NA 149 | Microbiocide | Dithiocarbamate |
| dithiocarbamate | | | | | |
| Formetanate | 22259-30-9 | 5E+05 | NA 150 | Insecticide | N-Methyl Carbamate |
| Formetanate hydrochloride | 23422-53-9 | 97301 | 111 | Insecticide | N-Methyl Carbamate |
| Furathiocarb | 65907-30-4 | NA 176 | NA 52 | Insecticide | Thiocarbamate |
| Isolan | 119-38-0 | 5E+05 | NA 155 | Insecticide | Other Carbamate |
| Isopolinate | 3134-70-1 | NA 232 | NA 232 | Herbicide | Thiocarbamate |
| Isoprocarb | 2631-40-5 | 5E+05 | NA 53 | Insecticide | N-Methyl Carbamate |
| Karbutilate | 4849-32-5 | 97401 | 691 | Herbicide | Other Carbamate |
| m-Cumenyl methylcarbamate | 64-00-6 | 47801 | NA 414 | Insecticide | N-Methyl Carbamate |
| Mancopper | 53988-93-5 | NA 181 | NA 180 | Fungicide | Dithiocarbamate |
| Mancozeb | ######## | 14504 | 211 | Fungicide | Dithiocarbamate, |
| | | | | | Inorganic-Zinc |
| Maneb | 12427-38-2 | 14505 | 369 | Fungicide | Dithiocarbamate |
| Maneb and nickel sulfate | 8005-46-7 | 6E+05 | NA 158 | Fungicide | Dithiocarbamate, |
| hexahydrate (014505 + 050505) | | | | | Inorganic-Nickel |
| Manganous | 15339-36-3 | 34802 | NA 304 | Fungicide | Dithiocarbamate |
| dimethyldithiocarbamate | | | | | |
| Mecarbinizid | 27386-64-7 | 6E+05 | NA 157 | N/A | Other Carbamate |
| Mercuric dimethyl | 15415-64-2 | 34808 | 709 | Microbiocide | Inorganic-Mercury, |
| dithiocarbamate | | | | | Heavy metal, |
| | | | | | Dithiocarbamate |
| Metam sodium, dihydrate | 137-42-8 | 39003 | NA 163 | Fumigant, | Dithiocarbamate |
| | | | | Fungicide, | |
| | | | | Microbiocide, | |
| | | | | Algaecide, | |
| | | | | Nematicide | |
| Metam-sodium | 6734-80-1 | 39003 | 616 | Fumigant, | Dithiocarbamate |
| | | | | Herbicide, | |
| | | | | Fungicide, | |
| | | | | Microbiocide, | |
| | | | | Algaecide, | |
| | | | | Nematicide | |
| Methasulfocarb | 66952-49-6 | NA 202 | NA 201 | Fungicide, | Thiocarbamate |
| | | | | Plant Growth | |
| | | | | Regulator | |
| Methiobencarb | 18357-78-3 | NA 232 | NA 232 | Herbicide | Thiocarbamate |
| Methiocarb | 2032-65-7 | 1E+05 | 375 | Insecticide, | N-Methyl Carbamate |
| | | | | Molluscicide | |
| Methiocarb sulfone | 2179-25-1 | 2E+05 | 4077 | Breakdown | N-Methyl Carbamate |
| | | | | product | |
| Methiocarb sulfoxide | ######## | 2E+05 | 4078 | Breakdown | N-Methyl Carbamate |
| | | | | product | |
| Methomyl | 16752-77-5 | 90301 | 383 | Insecticide, | N-Methyl Carbamate |
| | | | | Breakdown | |
| | | | | product | |
| Metiram | 9006-42-2 | 14601 | 493 | Fungicide | Dithiocarbamate, |
| | | | | | Inorganic-Zinc |
| Metolcarb | 1129-41-5 | NA 202 | NA 201 | Insecticide | N-Methyl Carbamate |
| Mexacarbate | 315-18-4 | 44201 | 623 | Insecticide | N-Methyl Carbamate |
| Mobam | 1079-33-0 | 90401 | NA 848 | Insecticide | N-Methyl Carbamate |
| Molinate | 2212-67-1 | 41402 | 449 | Herbicide | Thiocarbamate |
| Molinate sulfoxide | NA 1052 | NA 156 | 4080 | Breakdown | Thiocarbamate |
| | | | | product | |
| Morpholinomethyl | 31848-11-0 | 6E+05 | NA 158 | Microbiocide | Dithiocarbamate |
| dimethyldithiocarbamate | | | | | |
| Nabam | 142-59-6 | 14503 | 417 | Fungicide, | Dithiocarbamate |
| | | | | Herbicide | |
| Nitrilacarb | 29672-19-3 | 2E+05 | NA 129 | Insecticide | Other Carbamate |
| o-(2-Propynyloxy)phenyl | 3279-46-7 | 3E+05 | NA 138 | Insecticide | N-Methyl Carbamate |
| methylcarbamate | | | | | |
| Orbencarb | 34622-58-7 | NA 189 | NA 188 | Herbicide | Thiocarbamate |
| Oxamyl | 23135-22-0 | 1E+05 | 1910 | Insecticide, | N-Methyl Carbamate |
| | | | | Nematicide | |
| Pebulate | 1114-71-2 | 41403 | 590 | Herbicide | Thiocarbamate |
| Phenisopham | 57375-63-0 | NA 209 | NA 208 | Herbicide | Bis-Carbamate |
| Phenmedipham | 13684-63-4 | 98701 | 675 | Herbicide | Bis-Carbamate |
| Phenmedipham-ethyl | 13684-44-1 | 4E+05 | NA 149 | Herbicide | Bis-Carbamate |
| Phenylmercuric | 32407-99-1 | 66008 | NA 555 | Microbiocide, | Organomercury, |
| dimethyldithiocarbamate | | | | Fungicide | Dithiocarbamate, |
| | | | | | Heavy metal |
| Pirimicarb | 23103-98-2 | 1E+05 | 1875 | Insecticide | N-Methyl Carbamate |
| Pirimicarb sulfone | NA 1050 | NA 155 | 4090 | Breakdown | N-Methyl Carbamate |
| | | | | product | |
| Pirimicarb sulfoxide | NA 1049 | NA 155 | 4091 | Breakdown | N-Methyl Carbamate |
| | | | | product | |
| Potassium ammonium | 22221-14-3 | 14507 | NA 141 | Microbiocide | Dithiocarbamate |
| ethylenebis(dithiocarbamate) | | | | | |
| Potassium asulam | 14089-43-1 | 1E+05 | NA 893 | Herbicide | Other Carbamate |
| Potassium dimethyl dithio | 128-03-0 | 34803 | 1934 | Microbiocide | Dithiocarbamate |
| carbamate | | | | | |
| Potassium N-hydroxymethyl- | 51026-28-9 | 1E+05 | 1824 | N/A | Dithiocarbamate |
| N-methyldithio carbamate | | | | | |
| Potassium N-methyldithio | 137-41-7 | 39002 | 970 | Fumigant, | Dithiocarbamate |
| carbamate | | | | Fungicide, | |
| | | | | Microbiocide, | |
| | | | | Algaecide, | |
| | | | | Nematicide | |
| Promacyl | 34264-24-9 | NA 209 | NA 209 | Insecticide | N-Methyl Carbamate |
| Promecarb | 2631-37-0 | 3E+05 | 4092 | Insecticide | N-Methyl Carbamate |
| Propamocarb | 24579-73-5 | 1E+05 | 2147 | Fungicide | Other Carbamate |
| Propamocarb hydrochloride | 25606-41-1 | 1E+05 | 4022 | Fungicide | Other Carbamate |
| Propham | 122-42-9 | 47601 | 339 | Herbicide, | Other Carbamate |
| | | | | Plant Growth | |
| | | | | Regulator | |
| Propineb | 12071-83-9 | 5E+05 | NA 155 | Fungicide, | Dithiocarbamate, |
| | | | | Microbiocide | Inorganic-Zinc |
| Propoxur | 114-26-1 | 47802 | 62 | Insecticide | N-Methyl Carbamate |
| Propoxur, other related | NA 1073 | NA 161 | 90062 | Insecticide | N-Methyl Carbamate |
| Prosulfocarb | 52888-80-9 | NA 185 | NA 184 | Herbicide | Thiocarbamate |
| Prothiocarb | 19622-08-3 | NA 183 | NA 182 | Fungicide | Thiocarbamate |
| Prothiocarb hydrochloride | 19622-19-6 | NA 200 | NA 199 | Fungicide | Thiocarbamate |
| Pyributicarb | 88678-67-5 | NA 229 | NA 229 | Herbicide | Thiocarbamate |
| Pyrolan | 87-47-8 | 6E+05 | NA 157 | Insecticide | Other Carbamate |
| S-tert-Butyl | 2212-63-7 | 2E+05 | NA 128 | Herbicide | Thiocarbamate |
| dipropylthiocarbamate | | | | | |
| Sodium dimethyl dithio | 128-04-1 | 34804 | 548 | Fungicide | Dithiocarbamate |
| carbamate | | | | | |
| Sulfallate | 95-06-7 | 39001 | 115 | Herbicide | Dithiocarbamate |
| Swep | 1918-18-9 | 84601 | 4098 | N/A | Other Carbamate |
| Terbutol | ######## | 38801 | 51 | Herbicide | N-Methyl Carbamate |
| Tert-butyl dimethyl trithio | 3304-97-0 | 34807 | 1383 | Rodent | Dithiocarbamate |
| peroxycarbamate | | | | Repellent | |
| Tert- | NA 1130 | NA 166 | 91383 | N/A | Dithiocarbamate |
| butyldimethyltrithioperoxycarbamate, | | | | | |
| other related | | | | | |
| Thiobencarb | 28249-77-6 | 1E+05 | 1933 | Herbicide | Thiocarbamate |
| Thiobencarb sulfoxide | NA 1042 | NA 139 | 2937 | Breakdown | Thiocarbamate |
| | | | | product | |
| Thiocarboxime | 25171-63-5 | 6E+05 | NA 156 | N/A | N-Methyl Carbamate |
| Thiodicarb | 59669-26-0 | 1E+05 | 2202 | Molluscicide, | Thiocarbamate |
| | | | | Insecticide | |
| Thiofanox | 39196-18-4 | 1E+05 | 2938 | Insecticide | N-Methyl Carbamate |
| Thiram | 137-26-8 | 79801 | 589 | Fungicide | Dithiocarbamate |
| Thiram and NAD and IBA and | 75497-92-6 | 56011 | NA 463 | Fungicide, | Dithiocarbamate |
| 2-methyl-1- | | | | Plant Growth | |
| naphthaleneacetamide and 2- | | | | Regulator | |
| methyl-1-naphthaleneacetic | | | | | |
| acid | | | | | |
| Tiocarbazil | 36756-79-3 | 1E+05 | NA 911 | Herbicide | Thiocarbamate |
| Triallate | 2303-17-5 | 78802 | 49 | Herbicide | Thiocarbamate |
| Tricopper dichloride | 7076-63-3 | 5E+05 | NA 152 | Microbiocide | Dithiocarbamate, |
| dimethyldithiocarbamate | | | | | Inorganic-Copper |
| Trimethacarb | 12407-86-2 | 1E+05 | 2962 | Insecticide, | N-Methyl Carbamate |
| | | | | Molluscicide, | |
| | | | | Dog and Cat | |
| | | | | Repellent | |
| Trimethacarb, (2,3,5)- | 2655-15-4 | 1E+05 | NA 882 | Insecticide, | N-Methyl Carbamate |
| component of | | | | Molluscicide | |
| Trimethacarb, (3,4,5)- | 2686-99-9 | 1E+05 | NA 881 | Insecticide, | N-Methyl Carbamate |
| component of | | | | Molluscicide | |
| Vernolate | 1929-77-7 | 41404 | 1987 | Herbicide | Thiocarbamate |
| XMC | 2655-14-3 | NA 176 | NA 54 | Insecticide | N-Methyl Carbamate |
| Xylylcarb | ######## | NA 176 | NA 55 | Insecticide | N-Methyl Carbamate |
| Zineb | 12122-67-7 | 14506 | 627 | Fungicide | Dithiocarbamate, |
| | | | | | Inorganic-Zinc |
| Zineb-ethylene thiuram | NA 1465 | NA 217 | NA 216 | Fungicide | Dithiocarbamate, |
| disulfide adduct | | | | | Inorganic-Zinc |
| Ziram | 137-30-4 | 34805 | 629 | Fungicide, | Dithiocarbamate, |
| | | | | Microbiocide, | Inorganic-Zinc |
| | | | | Dog and Cat | |
| | | | | Repellent | |
| Ziram, cyclohexylamine | 16509-79-8 | 34806 | 1328 | Dog and Cat | Dithiocarbamate, |
| complex | | | | Repellent, | Inorganic-Zinc |
| | | | | Fungicide | |
| Acetamiprid | 135410-20-7 | 99050 | NA 864 | Insecticide | Chloro-nicotinyl |
| Imidacloprid | 105827-78-9 | ##### | 3849 | Insecticide | Chloro-nicotinyl |
| Imidacloprid | 138261-41-3 | ##### | NA 111 | Insecticide | Chloro-nicotinyl |
| Thiacloprid | 111988-49-9 | NA 201 | NA 201 | Insecticide | Chloro-nicotinyl |
| Halofenozide | 112226-61-6 | ##### | 5019 | Insecticide | Diacylhydrazine |
| Methoxyfenozide | 161050-58-4 | ##### | NA 983 | Insecticide | Diacylhydrazine |
| Tebufenozide | 112410-23-8 | ##### | 3957 | Insecticide | Diacylhydrazine |
| Bacillus thuringiensis Cry1F | NA 1486 | 6481 | NA 220 | Insecticide | GE Crop |
| protein and the genetic material | | | | | |
| necessary for its production | | | | | |
| (plasmid insert PHI8999) in | | | | | |
| corn pla | | | | | |
| Bacillus thuringiensis Cry1Ab | NA 1487 | 6430 | NA 220 | Insecticide | GE Crop |
| Delta-Endotoxin and the | | | | | |
| Genetic Material Necessary for | | | | | |
| Its Production in Corn [MON | | | | | |
| 810] | | | | | |
| Bacillus thuringiensis delta | 68038-71-1 | NA 160 | 3954 | Insecticide | GE Crop |
| endotoxin produced in corn | | | | | |
| Bacillus thuringiensis subsp. | NA 1226 | 6463 | NA 170 | Insecticide | GE Crop |
| Kurstaki CryIA (c) delta- | | | | | |
| endotoxin and the genetic | | | | | |
| material necessary for its | | | | | |
| production in corn | | | | | |
| Bacillus thuringiensis subsp. | 68038-71-1 | 6444 | 5048 | Insecticide | GE Crop |
| Kurstaki, delta-endotoxin as | | | | | |
| produced in corn by an HD-1 | | | | | |
| gene, and its controlling | | | | | |
| sequences and | | | | | |
| Bacillus thuringiensis subsp. | NA 1233 | 6466 | NA 171 | Insecticide | GE Crop |
| Tolworthi Cry9C protein and | | | | | |
| the genetic material necessary | | | | | |
| for its production (pRVA9909) | | | | | |
| in corn p | | | | | |
| Bacillus thuringiensis | NA 1237 | 6432 | NA 171 | Insecticide | GE Crop |
| subspecies Tenebrionis delta | | | | | |
| endotoxin as produced in | | | | | |
| potato by Cry IIIA gene and its | | | | | |
| controlling sequenc | | | | | |
| Bacillus thuringiensis var. | NA 1480 | 6402 | 3554 | Insecticide | GE Crop |
| Kurstaki protein in cottonseed | | | | | |
| Bacillus thuringiensis, subsp. | 68038-71-1 | 6461 | 3986 | Insecticide | GE Crop |
| Kurstaki, strain HD-1, delta- | | | | | |
| endotoxin as produced in corn | | | | | |
| by a Cry 1A(b) gene and its | | | | | |
| controlling | | | | | |
| HD-1 Protein as encoded by | NA 1273 | 6408 | NA 173 | Insecticide | GE Crop |
| Bacillus thuringiensis subsp. | | | | | |
| kurstaki gene and produced in | | | | | |
| corn | | | | | |
| Plant pesticide Bacillus | NA 1285 | 6458 | NA 174 | Insecticide | GE Crop |
| thuringiensis Cry1A(b) delta- | | | | | |
| endotoxin and the genetic | | | | | |
| material necessary for its | | | | | |
| production (plasmid V | | | | | |
| Potato leafroll virus (PLRV) | NA 1295 | 6469 | NA 175 | Fungicide | GE Crop |
| replicase protein as produced in | | | | | |
| potato plants | | | | | |
| 1,1,1-trichloroethane | 71-55-6 | 81201 | 138 | Solvent | Halogenated organic |
| 1,1,2-trichloro-1,2,2-trifluoro- | 76-13-1 | NA 972 | 2960 | Solvent | Halogenated organic |
| ethane | | | | | |
| 1,1,2-trichloroethane | 79-00-5 | 81203 | 1425 | Solvent | Halogenated organic |
| 1,2,3,4-Tetrachlorobenzene | 634-66-2 | 61101 | NA 494 | N/A | Halogenated organic |
| 1,2,4-trichlorobenzene | 120-82-1 | 81101 | 600 | N/A | Halogenated organic |
| 1,2-dichloropropane | 78-87-5 | 29002 | 2501 | Fumigant, | Halogenated organic |
| | | | | Nematicide | |
| 1,2-Dichloropropane mixt. with | ######## | 29004 | NA 215 | Fumigant, | Halogenated organic |
| 1,3-dichloropropene and | | | | Nematicide | |
| methyl isothiocyanate | | | | | |
| 1,2-dichloropropane, 1,3- | NA 1022 | 29002 | 185 | Fumigant, | Halogenated organic |
| dichloropropene and related C3 | | | | Nematicide | |
| compounds | | | | | |
| 1,3-Dichloro-1-propene, mixt. | 8003-19-8 | 29003 | NA 214 | Fumigant, | Halogenated organic |
| with 1,2-dichloropropane | | | | Nematicide | |
| 1,3-dichloropropene | 542-75-6 | 29001 | 573 | Fumigant, | Halogenated organic |
| | | | | Nematicide | |
| 1-chloro-1,1-difluoro ethane | 75-68-3 | NA 423 | 3103 | Solvent | Halogenated organic |
| 1-chlorobutane | 109-69-3 | NA 425 | 1151 | Solvent | Halogenated organic |
| 3-chloro-2-methyl propene | 563-47-3 | NA 422 | 2463 | N/A | Halogenated organic |
| Bis(2,2-dichloroethyl)ether | 1191-17-9 | 29502 | NA 217 | N/A | Halogenated organic |
| Bromoethane | 74-96-4 | NA 131 | 2427 | N/A | Halogenated organic |
| Carbon tetrachloride | 56-23-5 | 16501 | 109 | Solvent, | Halogenated organic |
| | | | | Fumigant | |
| Carbon tetrachloride with EDB | 53908-27-3 | 16503 | NA 146 | Fumigant, | Halogenated organic |
| & EDC | | | | Nematicide | |
| Carbon tetrachloride with EDC | ######## | 16502 | NA 145 | Fumigant, | Halogenated organic |
| | | | | Nematicide | |
| Carbon tetrafluoride | 75-73-0 | NA 113 | 3099 | Propellant | Halogenated organic |
| Chloro difluoro methane | 75-45-6 | 15 | 3104 | Propellant, | Halogenated organic |
| | | | | Fumigant, | |
| | | | | Insecticide | |
| Chlorodibromomethane | 124-48-1 | NA 426 | 2462 | N/A | Halogenated organic |
| Chloroform | 67-66-3 | 20701 | 133 | Solvent, | Halogenated organic |
| | | | | Fumigant | |
| DBCP | 96-12-8 | 11301 | 183 | Fumigant, | Halogenated organic |
| | | | | Nematicide | |
| DBCP, other related | NA 1079 | NA 161 | 90183 | Fumigant, | Halogenated organic |
| | | | | Nematicide | |
| Dichloroethylene, 1,1- | 75-35-4 | 6E+05 | NA 13 | Impurity | Halogenated organic |
| Dichlorofluoromethane | 75-43-4 | NA 348 | 1815 | Propellant | Halogenated organic |
| Difluoro diphenyl | 475-26-3 | 32001 | NA 293 | N/A | Halogenated organic |
| trichloroethane | | | | | |
| Ethane, 1,1,1,2-tetrafluoro- | 811-97-2 | 1E+05 | NA 873 | N/A | Halogenated organic |
| Ethyl chloride | 75-00-3 | NA 114 | 3198 | N/A | Halogenated organic |
| Ethylene chlorobromide | 107-04-0 | 42001 | 2557 | N/A | Halogenated organic |
| Ethylene dibromide | 106-93-4 | 42002 | 271 | Fumigant, | Halogenated organic |
| | | | | Nematicide | |
| Ethylene dichloride | 107-06-2 | 42003 | 274 | Fumigant, | Halogenated organic |
| | | | | Insecticide | |
| Freon 11 | 75-69-4 | 13 | 1460 | Propellant, | Halogenated organic |
| | | | | Fumigant, | |
| | | | | Insecticide | |
| Freon 12 | 75-71-8 | 14 | 1459 | Propellant, | Halogenated organic |
| | | | | Fumigant, | |
| | | | | Insecticide | |
| Halazone | 80-13-7 | 77201 | 316 | N/A | Halogenated organic |
| Hexachloroacetone | 116-16-5 | 43701 | 320 | Herbicide | Halogenated organic |
| Hexachloroacetone, other | NA 1090 | NA 162 | 90320 | Herbicide | Halogenated organic |
| related | | | | | |
| Hexachloroethane | 67-72-1 | 45201 | NA 22 | N/A | Halogenated organic |
| Hydrofluorocarbon 152A | NA 617 | NA 112 | 2599 | Propellant | Halogenated organic |
| Methyl bromide | 74-83-9 | 53201 | 385 | Fumigant, | Halogenated organic |
| | | | | Insecticide, | |
| | | | | Herbicide, | |
| | | | | Nematicide | |
| Methyl chloride | 74-87-3 | 53202 | 2677 | N/A | Halogenated organic |
| Methyl iodide | 74-88-4 | NA 177 | NA 165 | Fumigant, | Halogenated organic |
| | | | | Nematicide | |
| Methylene chloride | 75-09-2 | 42004 | 388 | Solvent, Dog | Halogenated organic |
| | | | | and Cat | |
| | | | | Repellent | |
| Ortho-dichlorobenzene | 95-50-1 | 59401 | 578 | Insecticide | Halogenated organic |
| Ortho-dichlorobenzene, other | NA 1116 | NA 165 | 90683 | Insecticide | Halogenated organic |
| related | | | | | |
| Para-dichlorobenzene | 106-46-7 | 61501 | 455 | Insecticide, | Halogenated organic |
| | | | | Insect | |
| | | | | Repellent, | |
| | | | | Rodenticide, | |
| | | | | Fungicide | |
| Pentachloroethane | 76-01-7 | 6E+05 | NA 159 | N/A | Halogenated organic |
| Propargyl bromide | 106-96-7 | 68701 | NA 608 | Fumigant, | Halogenated organic |
| | | | | Nematicide | |
| Tetrachloroethane | 79-34-5 | 78601 | 3559 | Solvent, | Halogenated organic |
| | | | | Fumigant | |
| Tetrachloroethylene | 127-18-4 | 78501 | 1174 | Solvent | Halogenated organic |
| Tetraiodo ethylene | 513-92-8 | 46908 | 582 | N/A | Halogenated organic |
| trans-1,3-Dichloropropene | 10061-02-6 | NA 227 | NA 227 | Fumigant | Halogenated organic |
| Trichloro ethylene | 79-01-6 | 81202 | 595 | N/A | Halogenated organic |
| Trichlorobenzene | 87-61-6 | NA 971 | 1619 | N/A | Halogenated organic |
| Trichlorofluoromethane | 75-69-4 | 13 | 3482 | Propellant, | Halogenated organic |
| | | | | Fumigant, | |
| | | | | Insecticide | |
| Trichloromethane | 67-66-3 | 20701 | 1758 | Solvent, | Halogenated organic |
| | | | | Fumigant | |
| 2,3(and 3,4)-Dichlorobenzyl | 62046-37-1 | ##### | NA 136 | Insecticide | N-Methyl Carbamate |
| methylcarbamates | | | | | |
| 2,4-Dimethyl-1,3-dithiolane-2- | 26419-73-8 | ##### | NA 143 | Insecticide | N-Methyl Carbamate |
| carboxaldehyde O- | | | | | |
| (methylcarbamoyl)oxime | | | | | |
| 2-(4,5-Dimethyl-1,3-dioxolan- | 1905930 | ##### | NA 145 | Insecticide | N-Methyl Carbamate |
| 2-yl)phenyl-N- | | | | | |
| methylcarbamate | | | | | |
| 2-chloro-4,5-xylyl N-hydroxy- | 14357-82-5 | ##### | NA 117 | Insecticide | N-Methyl Carbamate |
| N-methylcarbamate | | | | | |
| 2-cyclopentylphenyl | 3282-00-6 | ##### | NA 132 | Insecticide | N-Methyl Carbamate |
| methylcarbamate | | | | | |
| 2-Hydroxyphenyl | 10309-97-4 | ##### | NA 118 | Insecticide | N-Methyl Carbamate |
| methylcarbamate | | | | | |
| 3,4-Dichlorobenzyl | 2328-31-6 | ##### | NA 136 | Insecticide | N-Methyl Carbamate |
| methylcarbamate | | | | | |
| 3,5-Diisopropylphenyl | 330-64-3 | ##### | NA 142 | Insecticide | N-Methyl Carbamate |
| methylcarbamate | | | | | |
| 3-hydroxycarbofuran | 16655-82-6 | ##### | 4074 | Breakdown | N-Methyl Carbamate |
| | | | | product | |
| 3-ketocarbofuran | 16709-30-1 | NA 176 | NA 164 | Breakdown | N-Methyl Carbamate |
| | | | | product | |
| 4- | 17702-57-7 | ##### | NA 142 | Insecticide | N-Methyl Carbamate |
| (((Dimethylamino)methylene)amino)- | | | | | |
| m-tolyl | | | | | |
| methylcarbamate | | | | | |
| 4-(Methylamino)-3,5-xylyl | 10389-50-1 | ##### | NA 118 | Insecticide | N-Methyl Carbamate |
| methylcarbamate | | | | | |
| 4-(Methylformamido)-3,5- | 10233-94-0 | ##### | NA 118 | Insecticide | N-Methyl Carbamate |
| xylyl methylcarbamate | | | | | |
| 4-(Methylthio)-m-tolyl | 3566-00-5 | ##### | NA 157 | Insecticide | N-Methyl Carbamate |
| methylcarbamate | | | | | |
| 4-Amino-3,5-xylyl | 831-76-5 | ##### | NA 117 | Insecticide | N-Methyl Carbamate |
| methylcarbamate | | | | | |
| 5,6,7,8-Tetrahydro-1-naphthyl | 1136-84-1 | ##### | NA 148 | Insecticide | N-Methyl Carbamate |
| methylcarbamate | | | | | |
| 6-(and 2)-chloro-3,4-xylyl | 8063-85-2 | 38701 | NA 322 | Insecticide | N-Methyl Carbamate |
| methylcarbamate | | | | | |
| Aldicarb | 116-06-3 | 98301 | 575 | Insecticide, | N-Methyl Carbamate |
| | | | | Nematicide | |
| Aldicarb sulfoxide | 1646-87-3 | ##### | 2361 | Breakdown | N-Methyl Carbamate |
| | | | | product | |
| Aldoxycarb | 1646-88-4 | ##### | 2265 | Breakdown | N-Methyl Carbamate |
| | | | | product | |
| Allyxycarb | 6392-46-7 | ##### | NA 133 | Insecticide | N-Methyl Carbamate |
| Aminocarb | 2032-59-9 | 44401 | 2435 | Insecticide | N-Methyl Carbamate |
| Bendiocarb | 22781-23-3 | ##### | 1924 | Insecticide | N-Methyl Carbamate |
| Bufencarb | 2282-34-0 | 59302 | 91 | Insecticide | N-Methyl Carbamate |
| Bufencarb | 8065-36-9 | 59303 | NA 485 | Insecticide | N-Methyl Carbamate |
| Bufencarb, component of (with | 672-04-8 | 59301 | NA 484 | Insecticide | N-Methyl Carbamate |
| 059302) | | | | | |
| Butacarb | 2655-19-8 | ##### | NA 135 | Insecticide | N-Methyl Carbamate |
| Butocarboxim | 34681-10-2 | NA 175 | NA 49 | Insecticide | N-Methyl Carbamate |
| Butoxycarboxim | 34681-23-7 | ##### | 2201 | Insecticide | N-Methyl Carbamate |
| Carbanolate | 671-04-5 | ##### | NA 124 | Insecticide | N-Methyl Carbamate |
| Carbaryl | 63-25-2 | 56801 | 105 | Insecticide, | N-Methyl Carbamate |
| | | | | Plant Growth | |
| | | | | Regulator, | |
| | | | | Nematicide | |
| Carbofuran | 1563-66-2 | 90601 | 106 | Insecticide, | N-Methyl Carbamate |
| | | | | Nematicide | |
| Carbosulfan | 55285-14-8 | 90602 | 2182 | Insecticide | N-Methyl Carbamate |
| Cloethocarb | 51487-69-5 | ##### | NA 100 | Insecticide, | N-Methyl Carbamate |
| | | | | Molluscicide | |
| Dichlormate | 1966-58-1 | ##### | 690 | Herbicide | N-Methyl Carbamate |
| Dioxacarb | 6988-21-2 | ##### | 4067 | Insecticide | N-Methyl Carbamate |
| Ethiofencarb | 29973-13-5 | ##### | NA 50 | Insecticide | N-Methyl Carbamate |
| Fenethacarb | 30087-47-9 | ##### | NA 141 | Insecticide | N-Methyl Carbamate |
| Fenobucarb | 3766-81-2 | NA 176 | NA 51 | Insecticide | N-Methyl Carbamate |
| Formetanate | 22259-30-9 | ##### | NA 150 | Insecticide | N-Methyl Carbamate |
| Formetanate hydrochloride | 23422-53-9 | 97301 | 111 | Insecticide | N-Methyl Carbamate |
| Isoprocarb | 2631-40-5 | ##### | NA 53 | Insecticide | N-Methyl Carbamate |
| m-Cumenyl methylcarbamate | 64-00-6 | 47801 | NA 414 | Insecticide | N-Methyl Carbamate |
| Methiocarb | 2032-65-7 | ##### | 375 | Insecticide, | N-Methyl Carbamate |
| | | | | Molluscicide | |
| Methiocarb sulfone | 2179-25-1 | ##### | 4077 | Breakdown | N-Methyl Carbamate |
| | | | | product | |
| Methiocarb sulfoxide | 267266 | ##### | 4078 | Breakdown | N-Methyl Carbamate |
| | | | | product | |
| Methomyl | 16752-77-5 | 90301 | 383 | Insecticide, | N-Methyl Carbamate |
| | | | | Breakdown | |
| | | | | product | |
| Metolcarb | 1129-41-5 | NA 202 | NA 201 | Insecticide | N-Methyl Carbamate |
| Mexacarbate | 315-18-4 | 44201 | 623 | Insecticide | N-Methyl Carbamate |
| Mobam | 1079-33-0 | 90401 | NA 848 | Insecticide | N-Methyl Carbamate |
| o-(2-Propynyloxy)phenyl | 3279-46-7 | ##### | NA 138 | Insecticide | N-Methyl Carbamate |
| methylcarbamate | | | | | |
| Oxamyl | 23135-22-0 | ##### | 1910 | Insecticide, | N-Methyl Carbamate |
| | | | | Nematicide | |
| Pirimicarb | 23103-98-2 | ##### | 1875 | Insecticide | N-Methyl Carbamate |
| Pirimicarb sulfone | NA 1050 | NA 155 | 4090 | Breakdown | N-Methyl Carbamate |
| | | | | product | |
| Pirimicarb sulfoxide | NA 1049 | NA 155 | 4091 | Breakdown | N-Methyl Carbamate |
| | | | | product | |
| Promacyl | 34264-24-9 | NA 209 | NA 209 | Insecticide | N-Methyl Carbamate |
| Promecarb | 2631-37-0 | ##### | 4092 | Insecticide | N-Methyl Carbamate |
| Propoxur | 114-26-1 | 47802 | 62 | Insecticide | N-Methyl Carbamate |
| Propoxur, other related | NA 1073 | NA 161 | 90062 | Insecticide | N-Methyl Carbamate |
| Terbutol | 5419 | 38801 | 51 | Herbicide | N-Methyl Carbamate |
| Thiocarboxime | 25171-63-5 | ##### | NA 156 | N/A | N-Methyl Carbamate |
| Thiofanox | 39196-18-4 | ##### | 2938 | Insecticide | N-Methyl Carbamate |
| Trimethacarb | 12407-86-2 | ##### | 2962 | Insecticide, | N-Methyl Carbamate |
| | | | | Molluscicide, | |
| | | | | Dog and Cat | |
| | | | | Repellent | |
| Trimethacarb, (2,3,5)- | 2655-15-4 | ##### | NA 882 | Insecticide, | N-Methyl Carbamate |
| component of | | | | Molluscicide | |
| Trimethacarb, (3,4,5)- | 2686-99-9 | ##### | NA 881 | Insecticide, | N-Methyl Carbamate |
| component of | | | | Molluscicide | |
| XMC | 2655-14-3 | NA 176 | NA 54 | Insecticide | N-Methyl Carbamate |
| Xylylcarb | 190572 | NA 176 | NA 55 | Insecticide | N-Methyl Carbamate |
| Aldrin | 309-00-2 | 45101 | 9 | Insecticide | Organochlorine |
| Aldrin, other related | NA 1069 | NA 160 | 90009 | Insecticide | Organochlorine |
| alpha-BHC | 319-84-6 | NA 200 | 767 | Insecticide | Organochlorine |
| Arochlor (composition | 12767-79-2 | 17803 | NA 161 | N/A | Organochlorine |
| unspecified, PCBs and/or | | | | | |
| PCTs) | | | | | |
| Aroclor | 1336-36-3 | 17801 | 5003 | N/A | Organochlorine |
| Bandane | 8029-29-6 | 20301 | 54 | Insecticide | Organochlorine |
| BHC (other than gamma | NA 1092 | NA 162 | 90359 | Insecticide | Organochlorine |
| isomer) | | | | | |
| BHC, beta isomer | 319-85-7 | NA 175 | NA 46 | Insecticide | Organochlorine |
| Chlordane | 57-74-9 | 58201 | 130 | Insecticide | Organochlorine |
| Chlordane, other related | NA 1075 | NA 161 | 90130 | Insecticide | Organochlorine |
| Chlordane, technical | 12789-03-6 | 58202 | NA 477 | Insecticide | Organochlorine |
| Chlordecone | 143-50-0 | 27701 | 347 | Insecticide | Organochlorine |
| Chlorendic Acid | 115-28-6 | NA 228 | NA 228 | N/A | Organochlorine |
| DDD | 72-54-8 | 29101 | 3002 | Insecticide | Organochlorine |
| DDD | 6088-31-3 | NA 415 | 184 | Breakdown | Organochlorine |
| | | | | product | |
| DDD, other related | NA 1080 | NA 161 | 90184 | Breakdown | Organochlorine |
| | | | | product | |
| DDE | 72-55-9 | NA 129 | 2092 | Breakdown | Organochlorine |
| | | | | product | |
| DDT | 50-29-3 | 29201 | 186 | Insecticide | Organochlorine |
| Dicofol | 115-32-2 | 10501 | 346 | Insecticide | Organochlorine |
| Dieldrin | 60-57-1 | 45001 | 210 | Insecticide, | Organochlorine |
| | | | | Breakdown | |
| | | | | product | |
| Dieldrin, other related | NA 1085 | NA 162 | 90210 | Insecticide | Organochlorine |
| Dienochlor | 2227-17-0 | 27501 | 468 | Insecticide | Organochlorine |
| Dioxin (2,3,7,8-TCDD) | 1746-01-6 | ##### | 4068 | Impurity | Organochlorine |
| Dioxin (other than 2,3,7,8- | NA 1319 | NA 177 | NA 176 | Impurity | Organochlorine |
| TCDD) | | | | | |
| Endosulfan | 115-29-7 | 79401 | 259 | Insecticide | Organochlorine |
| Endosulfan I (alpha) | 959-98-8 | 79402 | NA 771 | Insecticide | Organochlorine |
| Endosulfan II (beta) | 33213-65-9 | 79403 | 4046 | Insecticide | Organochlorine |
| Endosulfan sulfate | 1031-07-8 | 79404 | 4047 | Breakdown | Organochlorine |
| | | | | product | |
| Endrin | 72-20-8 | 41601 | 262 | Insecticide, | Organochlorine |
| | | | | Avicide | |
| Endrin aldehyde | 7421-93-4 | NA 156 | 4069 | Breakdown | Organochlorine |
| | | | | product | |
| Endrin ketone | 53494-70-5 | 41601 | 5029 | Breakdown | Organochlorine |
| | | | | product | |
| Endrin, other related | NA 1086 | NA 162 | 90262 | Insecticide | Organochlorine |
| Heptachlor | 76-44-8 | 44801 | 317 | Insecticide | Organochlorine |
| Heptachlor epoxide | 1024-57-3 | 44801 | 4073 | Breakdown | Organochlorine |
| | | | | product | |
| Heptachlor, other related | NA 1089 | NA 162 | 90317 | Insecticide | Organochlorine |
| Hexachloro dibenzo-p-dioxin | 34465-46-8 | ##### | 2587 | Impurity | Organochlorine |
| Hexachlorobenzene | 118-74-1 | 61001 | 321 | Microbiocide, | Organochlorine |
| | | | | Fungicide, | |
| | | | | Insecticide | |
| Hexachlorocyclohexane | 608-73-1 | 8901 | NA 20 | Insecticide | Organochlorine |
| Hexachlorocyclopentadiene | 77-47-4 | 27502 | NA 21 | Insecticide | Organochlorine |
| Hexachlorodibenzofuran (as | 55684-94-1 | ##### | NA 161 | Impurity | Organochlorine |
| impurity) | | | | | |
| Isodrin | 465-73-6 | NA 208 | NA 207 | Insecticide | Organochlorine |
| Kelevan | 4234-79-1 | NA 208 | NA 208 | Insecticide | Organochlorine |
| Lindane | 58-89-9 | 9001 | 359 | Insecticide, | Organochlorine |
| | | | | Rodenticide | |
| Methoxychlor | 72-43-5 | 34001 | 384 | Insecticide | Organochlorine |
| Methoxychlor, other related | NA 1093 | NA 162 | 90384 | Insecticide | Organochlorine |
| Mirex | 2385-85-5 | 39201 | 402 | Insecticide | Organochlorine |
| Nonachlor | 3734-49-4 | 44802 | 5448 | Insecticide | Organochlorine |
| Oxychlordane | 27304-13-8 | NA 177 | NA 177 | Breakdown | Organochlorine |
| | | | | product | |
| Oxychlordane, mix of isomers | 26880-48-8 | NA 177 | NA 177 | Breakdown | Organochlorine |
| | | | | product | |
| Strobane | 8001-50-1 | 20401 | 553 | Insecticide | Organochlorine |
| Toxaphene | 8001-35-2 | 80501 | 594 | Insecticide | Organochlorine |
| trans-Nonachlor | 39765-80-5 | NA 177 | NA 177 | Insecticide | Organochlorine |
| (E)-Mevinphos | 298-01-1 | 15802 | NA 143 | Insecticide | Organophosphorus |
| (Z)-Mevinphos | 338-45-4 | 15803 | NA 144 | Insecticide | Organophosphorus |
| 2,5-dichloro-alpha- | 2701-86-2 | ##### | NA 116 | Insecticide | Organophosphorus |
| (chloromethylene)benzyl | | | | | |
| diethyl phosphate | | | | | |
| 2,5-dichloro-alpha- | 5723-62-6 | ##### | NA 116 | Insecticide | Organophosphorus |
| (dichloromethylene)benzyl | | | | | |
| diethyl phosphate | | | | | |
| Acephate | 30560-19-1 | ##### | 1685 | Insecticide | Organophosphorus |
| Acethion | 919-54-0 | ##### | NA 139 | Insecticide | Organophosphorus |
| Acetoxon | 2425-25-4 | ##### | NA 139 | Insecticide, | Organophosphorus |
| | | | | Breakdown | |
| | | | | product | |
| Akton | 1757-18-2 | 84901 | 1421 | Insecticide | Organophosphorus |
| Akton, other related | NA 1131 | NA 166 | 91421 | Insecticide | Organophosphorus |
| alpha,alpha-Dithiol bis(O,O- | 2782-70-9 | ##### | NA 150 | N/A | Organophosphorus |
| dimethyl | | | | | |
| phosphorodithioate)toluene | | | | | |
| Amidithion | 919-76-6 | 59601 | NA 487 | Insecticide | Organophosphorus |
| Amiton | 78-53-5 | 57302 | NA 473 | Insecticide | Organophosphorus |
| Amiton oxalate | 3734-97-2 | 57301 | NA 472 | Insecticide | Organophosphorus |
| Anilofos | 64249-01-0 | NA 202 | NA 201 | Insecticide | Organophosphorus |
| Athidathion | 19691-80-6 | ##### | NA 141 | Insecticide | Organophosphorus |
| Azamethiphos | 35575-96-3 | ##### | NA 111 | Insecticide | Organophosphorus |
| Azethion | 72348-92-6 | ##### | NA 139 | Insecticide | Organophosphorus |
| Azinphos-ethyl | 2642-71-9 | 58002 | 4053 | Insecticide | Organophosphorus |
| Azinphos-methyl | 86-50-0 | 58001 | 314 | Insecticide | Organophosphorus |
| Azinphos-methyl oxygen | NA 1057 | NA 157 | 4054 | Breakdown | Organophosphorus |
| analog | | | | product | |
| Azothoate | 5834-96-8 | ##### | NA 118 | Insecticide | Organophosphorus |
| Bensulide | 741-58-2 | 9801 | 70 | Herbicide | Organophosphorus |
| Bomyl | 122-10-1 | 84201 | 77 | Insecticide | Organophosphorus |
| Bromophos | 2104-96-3 | 8706 | NA 106 | Insecticide | Organophosphorus |
| Bromophos-ethyl | 4824-78-6 | ##### | NA 122 | Insecticide | Organophosphorus |
| Butamifos | 36335-67-8 | NA 202 | NA 201 | Herbicide | Organophosphorus |
| Butathiofos | 90338-20-8 | ##### | 2433 | Insecticide | Organophosphorus |
| Butonate | 126-22-7 | 35701 | 255 | N/A | Organophosphorus |
| Cadusafos | 95465-99-9 | ##### | NA 14 | Insecticide | Organophosphorus |
| Carbophenothion | 786-19-6 | 58102 | 110 | Insecticide | Organophosphorus |
| Chlorethoxyphos | 54593-83-8 | ##### | 5106 | Insecticide | Organophosphorus |
| Chlorfenvinphos | 470-90-6 | 84101 | 306 | Insecticide | Organophosphorus |
| Chlormephos | 24934-91-6 | ##### | NA 136 | Insecticide | Organophosphorus |
| Chlorphoxim | 14816-20-7 | NA 207 | NA 206 | Insecticide | Organophosphorus |
| Chlorprazophos | 36145-08-1 | ##### | NA 136 | Insecticide | Organophosphorus |
| Chlorpyrifos | 2921-88-2 | 59101 | 253 | Insecticide, | Organophosphorus |
| | | | | Nematicide | |
| Chlorpyrifos oxygen analog | 5598-15-2 | 59101 | 5034 | Insecticide, | Organophosphorus |
| | | | | Breakdown | |
| | | | | product | |
| Chlorpyrifos-methyl | 5598-13-0 | 59102 | 2468 | Insecticide | Organophosphorus |
| Chlorthion | 500-28-7 | 34501 | NA 300 | Insecticide | Organophosphorus |
| Chlorthiophos | 21923-23-9 | ##### | 2469 | Insecticide | Organophosphorus |
| Chlorthiophos | 60238-56-4 | ##### | NA 919 | Insecticide | Organophosphorus |
| Chlorthiophos II | 77503-29-8 | ##### | NA 918 | Insecticide | Organophosphorus |
| Chlorthiophos III | 77503-28-7 | ##### | NA 917 | Insecticide | Organophosphorus |
| cis-Azodrin | 919-44-8 | 58902 | NA 482 | Insecticide | Organophosphorus |
| cis-Methocrotophos | 69632-93-5 | ##### | NA 143 | Insecticide | Organophosphorus |
| Coumaphos | 56-72-4 | 36501 | 165 | Insecticide | Organophosphorus |
| Coumaphos, other related | NA 1077 | NA 161 | 90165 | Insecticide | Organophosphorus |
| Coumithioate | 572-48-5 | ##### | NA 141 | Insecticide | Organophosphorus |
| Crotoxyphos | 7700-17-6 | 58801 | 140 | Insecticide | Organophosphorus |
| Crotoxyphos, other related | NA 1076 | NA 161 | 90140 | Insecticide | Organophosphorus |
| Cyanofenphos | 13067-93-1 | ##### | NA 131 | Insecticide | Organophosphorus |
| Cyanophos | 2636-26-2 | ##### | NA 131 | Insecticide | Organophosphorus |
| Cyanthoate | 3734-95-0 | ##### | NA 131 | Insecticide | Organophosphorus |
| Cythioate | 115-93-5 | 59501 | NA 486 | Insecticide | Organophosphorus |
| DDVP | 62-73-7 | 84001 | 187 | Insecticide, | Organophosphorus |
| | | | | Breakdown | |
| | | | | product | |
| DDVP, other related | NA 1081 | 84001 | 90187 | Insecticide | Organophosphorus |
| Demephion-O | 682-80-4 | ##### | NA 143 | Insecticide, | Organophosphorus |
| | | | | Breakdown | |
| | | | | product | |
| Demephion-S | 2587-90-8 | ##### | NA 143 | Insecticide | Organophosphorus |
| Demeton | 8065-48-3 | 57601 | 566 | Insecticide, | Organophosphorus |
| | | | | Nematicide | |
| Demeton-O | 298-03-3 | 57602 | NA 474 | Insecticide | Organophosphorus |
| Demeton-O-methyl | 867-27-6 | 58703 | NA 480 | Insecticide | Organophosphorus |
| Demeton-S | 126-75-0 | 57603 | NA 475 | Insecticide | Organophosphorus |
| Demeton-S-methyl | 919-86-8 | NA 200 | NA 200 | Insecticide | Organophosphorus |
| Demeton-S-methyl (mixture) | 8022-00-2 | 58701 | 4063 | Insecticide | Organophosphorus |
| Demeton-S-methyl sulfone | 17040-19-6 | NA 183 | NA 182 | Breakdown | Organophosphorus |
| | | | | product, | |
| | | | | Insecticide | |
| Dequest 2010 phosphonate | NA 579 | NA 105 | 2496 | N/A | Organophosphorus |
| Dialifor | 10311-84-9 | ##### | 1799 | Insecticide | Organophosphorus |
| Dialifor, other related | NA 1138 | NA 167 | 91799 | Insecticide | Organophosphorus |
| Diamidafos | 1754-58-1 | ##### | NA 879 | Insecticide, | Organophosphorus |
| | | | | Nematicide | |
| Diazinon | 333-41-5 | 57801 | 198 | Insecticide | Organophosphorus |
| Diazoxon | 962-58-3 | NA 156 | 4064 | Breakdown | Organophosphorus |
| | | | | product | |
| Dichlofenthion | 97-17-6 | 28601 | 614 | Insecticide, | Organophosphorus |
| | | | | Nematicide | |
| Dicrotophos | 141-66-2 | 35201 | 72 | Insecticide | Organophosphorus |
| Dimethoate | 60-51-5 | 35001 | 216 | Insecticide | Organophosphorus |
| Dimethoate-ethyl | 116-01-8 | ##### | NA 147 | Insecticide | Organophosphorus |
| Dioxabenzofos | 3811-49-2 | ##### | NA 112 | Insecticide | Organophosphorus |
| Dioxathion | 78-34-2 | 37801 | 192 | Insecticide | Organophosphorus |
| Dioxathion, other related | NA 1083 | NA 161 | 90192 | Insecticide | Organophosphorus |
| Diphenprofos | 59010-86-5 | ##### | NA 920 | Insecticide | Organophosphorus |
| Disulfoton | 298-04-4 | 32501 | 230 | Insecticide, | Organophosphorus |
| | | | | Nematicide | |
| Disulfoton sulfone | 216746 | ##### | NA 140 | Insecticide | Organophosphorus |
| Disulfoton sulfoxide | 216777 | ##### | NA 140 | Breakdown | Organophosphorus |
| | | | | product | |
| Ditalimfos | 5131-24-8 | ##### | NA 891 | Fungicide | Organophosphorus |
| DMCP | 3309-87-3 | ##### | NA 119 | Insecticide | Organophosphorus |
| Edifenphos | 17109-49-8 | ##### | 1964 | Insecticide | Organophosphorus |
| Endothion | 319315 | ##### | NA 146 | Insecticide | Organophosphorus |
| EPBP | 3792-59-4 | ##### | NA 155 | Insecticide | Organophosphorus |
| EPN | 2104-64-5 | 41801 | 263 | Insecticide | Organophosphorus |
| Ethephon | 16672-87-0 | 99801 | 1626 | Plant Growth | Organophosphorus |
| | | | | Regulator | |
| Ethion | 563-12-2 | 58401 | 268 | Insecticide | Organophosphorus |
| Ethion, O-analog | 17356-42-2 | 58402 | NA 478 | Breakdown | Organophosphorus |
| | | | | product | |
| Ethoprop | 13194-48-4 | 41101 | 404 | Insecticide, | Organophosphorus |
| | | | | Nematicide | |
| Ethyl (2- | 5840-95-9 | ##### | NA 149 | Insecticide | Organophosphorus |
| mercaptoethyl)carbamate, S- | | | | | |
| ester of O,O-dimethyl | | | | | |
| phosphorodithioate | | | | | |
| Ethyl hydrogen 1- | 21921-96-0 | ##### | NA 148 | N/A | Organophosphorus |
| propylphosphonate | | | | | |
| Etrimfos | 38260-54-7 | ##### | NA 147 | Insecticide | Organophosphorus |
| Famphur | 52-85-7 | 59901 | 282 | Insecticide | Organophosphorus |
| Famphur, O-analog | 960-25-8 | 59902 | NA 491 | Breakdown | Organophosphorus |
| | | | | product | |
| Fenamiphos | 22224-92-6 | ##### | 1857 | Insecticide, | Organophosphorus |
| | | | | Nematicide | |
| Fenamiphos sulfone | 31972-44-8 | NA 156 | 4070 | Breakdown | Organophosphorus |
| | | | | product | |
| Fenamiphos sulfoxide | 31972-43-7 | NA 156 | 4071 | Breakdown | Organophosphorus |
| | | | | product | |
| Fenitrothion | 122-14-5 | ##### | 2520 | Insecticide | Organophosphorus |
| Fensulfothion | 115-90-2 | 32701 | 181 | Insecticide, | Organophosphorus |
| | | | | Nematicide | |
| Fenthion | 55-38-9 | 53301 | 63 | Insecticide, | Organophosphorus |
| | | | | Avicide | |
| Fenthion oxon | 3254-63-5 | ##### | NA 143 | Breakdown | Organophosphorus |
| | | | | product | |
| Fonofos | 944-22-9 | 41701 | 254 | Insecticide | Organophosphorus |
| Formothion | 2540-82-1 | ##### | NA 143 | Insecticide | Organophosphorus |
| Fosmethilan | 83733-82-8 | NA 208 | NA 207 | Insecticide | Organophosphorus |
| Fospirate | 5598-52-7 | ##### | 1856 | Insecticide | Organophosphorus |
| Fosthiazate | 98886-44-3 | ##### | NA 109 | N/A | Organophosphorus |
| Fosthietan | 21548-32-3 | ##### | NA 928 | Fumigant, | Organophosphorus |
| | | | | Nematicide | |
| Heptenophos | 23560-59-0 | ##### | NA 122 | Insecticide | Organophosphorus |
| Hexylthiofos | 41495-67-4 | ##### | NA 132 | Insecticide | Organophosphorus |
| Iodofenfos | 18181-70-9 | ##### | NA 138 | Insecticide | Organophosphorus |
| Iprobenfos | 26087-47-8 | NA 203 | NA 202 | Fungicide | Organophosphorus |
| IPSP | 1432970 | NA 208 | NA 207 | Insecticide | Organophosphorus |
| Isazophos | 42509-80-8 | ##### | 2282 | Insecticide | Organophosphorus |
| Isazophos-methyl | 42509-83-1 | ##### | NA 136 | Insecticide | Organophosphorus |
| Isocarbophos | 24353-61-5 | ##### | 2414 | Insecticide | Organophosphorus |
| Isochlorthion | 2463-84-5 | 34502 | NA 301 | Insecticide | Organophosphorus |
| Isofephos | 25311-71-1 | ##### | 2194 | Insecticide | Organophosphorus |
| Isothioate | 36614-38-7 | NA 208 | NA 207 | Insecticide | Organophosphorus |
| Isoxathion | 18854-01-8 | 57802 | NA 476 | Insecticide | Organophosphorus |
| Leptophos | 21609-90-5 | ##### | 1676 | Insecticide | Organophosphorus |
| Leptophos, other related | NA 1135 | NA 167 | 91676 | Insecticide | Organophosphorus |
| Lythidathion | 2669-32-1 | ##### | NA 147 | Insecticide | Organophosphorus |
| Malaoxon | NA 1054 | NA 156 | 4076 | Breakdown | Organophosphorus |
| | | | | product | |
| Malathion | 121-75-5 | 57701 | 367 | Insecticide | Organophosphorus |
| Mecarbam | 2595-54-2 | ##### | NA 140 | Insecticide | Organophosphorus |
| Mecarphon | 29173-31-7 | ##### | NA 156 | Insecticide | Organophosphorus |
| Menazon | 78-57-9 | ##### | NA 134 | N/A | Organophosphorus |
| Mephosfolan | 950-10-7 | ##### | NA 131 | Insecticide | Organophosphorus |
| Merphos | 150-50-5 | 74901 | 293 | Defoliant, Plant | Organophosphorus |
| | | | | Growth | |
| | | | | Regulator | |
| Merphos, other related | NA 1088 | NA 162 | 90293 | Defoliant, Plant | Organophosphorus |
| | | | | Growth | |
| | | | | Regulator | |
| Merpofos | 3568-56-7 | ##### | NA 129 | N/A | Organophosphorus |
| Methacrifos | 62610-77-9 | NA 189 | NA 188 | Insecticide | Organophosphorus |
| Methamidophos | 10265-92-6 | ##### | 1697 | Insecticide, | Organophosphorus |
| | | | | Breakdown | |
| | | | | product | |
| Methidathion | 950-37-8 | ##### | 1689 | Insecticide | Organophosphorus |
| Methidathion OA | NA 1065 | ##### | 5040 | Insecticide | Organophosphorus |
| Methyl paraoxon | 950-35-6 | 53502 | 4083 | Breakdown | Organophosphorus |
| | | | | product | |
| Methyl parathion | 298-00-0 | 53501 | 394 | Insecticide, | Organophosphorus |
| | | | | Nematicide | |
| Methyl parathion, other related | NA 1094 | NA 162 | 90394 | Insecticide, | Organophosphorus |
| | | | | Nematicide | |
| Methyl phenkapton | 3735-23-7 | ##### | NA 142 | N/A | Organophosphorus |
| Methyl trithion | 953-17-3 | 58101 | 3531 | Insecticide | Organophosphorus |
| Methyl-carbofenthion | NA 843 | NA 157 | 4057 | Insecticide | Organophosphorus |
| Mevinphos | 7786-34-7 | 15801 | 480 | Insecticide | Organophosphorus |
| Mevinphos (stereochemistry | 26718-65-0 | NA 200 | NA 200 | Insecticide | Organophosphorus |
| unspecified) | | | | | |
| Mevinphos, other related | NA 1103 | NA 163 | 90480 | Insecticide | Organophosphorus |
| Miral | 42509-80-8 | ##### | 2689 | Insecticide | Organophosphorus |
| Monocrotophos | 6923-22-4 | 58901 | 52 | Insecticide | Organophosphorus |
| Morphothion | 144-41-2 | ##### | NA 143 | Insecticide | Organophosphorus |
| Naled | 300-76-5 | 34401 | 418 | Insecticide | Organophosphorus |
| Naphthalophos | 1491-41-4 | ##### | NA 117 | Insecticide | Organophosphorus |
| O,O,S-Trimethyl | 2953-29-9 | ##### | NA 161 | Impurity | Organophosphorus |
| phosphorodithiate (as impurity) | | | | | |
| O,O-diethyl O- | 2668-92-0 | ##### | NA 138 | Insecticide | Organophosphorus |
| naphthaloximido | | | | | |
| phosphorothioate | | | | | |
| O,O-diethyl phosphoro | 226542 | NA 338 | 2508 | Insecticide | Organophosphorus |
| chloridothionate | | | | | |
| O,O-Diethyl S-(4,6-dimethyl- | 333-40-4 | ##### | NA 140 | Insecticide | Organophosphorus |
| 2-pyrimidinyl) | | | | | |
| phosphorodithioate | | | | | |
| O,O-diethyl-O-phenyl | 32345-29-2 | NA 337 | 2507 | Insecticide | Organophosphorus |
| phosphorothioate | | | | | |
| O,O-dimethyl S-(2- | 2674-91-1 | 58704 | NA 481 | Insecticide | Organophosphorus |
| (ethylsulfinyl)-1-methylethyl) | | | | | |
| phosphorothioate | | | | | |
| O-(2-chloro-4-nitrophenyl) O- | 328-04-1 | ##### | NA 137 | N/A | Organophosphorus |
| isopropyl | | | | | |
| ethylphosphonothioate | | | | | |
| O-(4-Nitrophenyl) O-phenyl | 2665-30-7 | ##### | NA 158 | N/A | Organophosphorus |
| methylphosphonothioate | | | | | |
| O-ethyl O-(4- | 2703-13-1 | ##### | NA 117 | N/A | Organophosphorus |
| (methylthio)phenyl) | | | | | |
| methylphosphonothioate | | | | | |
| O-Ethyl S-(4-methylphenyl) | 333-43-7 | ##### | NA 149 | N/A | Organophosphorus |
| ethylphosphonodithioate | | | | | |
| O-Methyl 2-nitro-4-tolyl | 36001-88-4 | ##### | NA 157 | N/A | Organophosphorus |
| isopropylphosphoramidothioate | | | | | |
| Omethoate | 1113-02-6 | 35002 | 2285 | Insecticide, | Organophosphorus |
| | | | | Breakdown | |
| | | | | product | |
| Oxydemeton-methyl | 301-12-2 | 58702 | 382 | Insecticide | Organophosphorus |
| Paraoxon | 311-45-5 | NA 155 | 4082 | Breakdown | Organophosphorus |
| | | | | product | |
| Parathion | 56-38-2 | 57501 | 459 | Insecticide | Organophosphorus |
| Parathion | 56-38-2 | 57401 | NA 174 | Insecticide, | Organophosphorus |
| | | | | Nematicide | |
| Parathion, other related | NA 1099 | NA 163 | 90459 | Insecticide, | Organophosphorus |
| | | | | Nematicide | |
| Phenkapton | 2275-14-1 | ##### | NA 139 | Insecticide | Organophosphorus |
| Phenthoate | 253180 | ##### | NA 889 | Insecticide | Organophosphorus |
| Phorate | 298-02-2 | 57201 | 478 | Insecticide, | Organophosphorus |
| | | | | Nematicide | |
| Phorate sulfone | 249924 | NA 155 | 4084 | Breakdown | Organophosphorus |
| | | | | product | |
| Phorate sulfoxide | 249892 | NA 155 | 4085 | Breakdown | Organophosphorus |
| | | | | product | |
| Phoratoxon | 2600-69-3 | NA 155 | 4086 | Breakdown | Organophosphorus |
| | | | | product | |
| Phoratoxon sulfone | NA 1051 | NA 155 | 4087 | Breakdown | Organophosphorus |
| | | | | product | |
| Phoratoxon sulfoxide | 249955 | NA 155 | 4088 | Breakdown | Organophosphorus |
| | | | | product | |
| Phosalone | 2310-17-0 | 97701 | 479 | Insecticide | Organophosphorus |
| Phosalone OA | NA 1066 | 97701 | 5041 | Insecticide, | Organophosphorus |
| | | | | Breakdown | |
| | | | | product | |
| Phosfolan | 947-02-4 | ##### | NA 131 | N/A | Organophosphorus |
| Phosmet | 732-11-6 | 59201 | 335 | Insecticide | Organophosphorus |
| Phosmetoxon | 3735-33-9 | 59202 | 4089 | Breakdown | Organophosphorus |
| | | | | product | |
| Phosnichlor | 5826-76-6 | 34503 | NA 302 | Insecticide | Organophosphorus |
| Phosphamidon | 13171-21-6 | 18201 | 482 | Insecticide | Organophosphorus |
| Phosphamidon, other related | NA 1104 | NA 163 | 90482 | Insecticide | Organophosphorus |
| Phostebupirim | 96182-53-5 | ##### | 5122 | Insecticide | Organophosphorus |
| Phoxim | 14816-18-3 | ##### | NA 159 | Insecticide | Organophosphorus |
| Phoxim-methyl | 14816-16-1 | ##### | NA 159 | Insecticide | Organophosphorus |
| Piperophos | 24151-93-7 | NA 219 | NA 218 | Herbicide | Organophosphorus |
| Pirimiphos ethyl | 23505-41-1 | ##### | 2781 | Insecticide | Organophosphorus |
| Pirimiphos ethyl, O-analog | 36378-61-7 | ##### | NA 899 | Breakdown | Organophosphorus |
| | | | | product | |
| Pirimiphos-methyl | 29232-93-7 | ##### | 2217 | Insecticide | Organophosphorus |
| Primidophos | 39247-96-6 | ##### | NA 127 | Insecticide | Organophosphorus |
| Profenofos | 41198-08-7 | ##### | 2042 | Insecticide | Organophosphorus |
| Propaphos | 7292-16-2 | NA 201 | NA 201 | Insecticide | Organophosphorus |
| Propetamphos | 31218-83-4 | ##### | 2122 | Insecticide | Organophosphorus |
| Propoxon | 5823-13-2 | ##### | NA 139 | Insecticide | Organophosphorus |
| Prothidathion | 20276-83-9 | ##### | NA 141 | Insecticide | Organophosphorus |
| Prothiofos | 34643-46-4 | ##### | 4094 | Insecticide | Organophosphorus |
| Prothion | 5969-94-8 | ##### | NA 139 | Insecticide | Organophosphorus |
| Prothoate | 2275-18-5 | ##### | NA 140 | Insecticide | Organophosphorus |
| Pyraclofos | 77458-01-6 | NA 183 | NA 182 | Insecticide | Organophosphorus |
| Pyrazophos | 13457-18-6 | ##### | NA 149 | Fungicide | Organophosphorus |
| Pyridiphenthion | 119-12-0 | 53302 | NA 450 | Insecticide | Organophosphorus |
| Quinalphos | 13593-03-8 | ##### | NA 145 | Insecticide | Organophosphorus |
| Quinalphos-methyl | 13593-08-3 | ##### | NA 145 | Insecticide | Organophosphorus |
| Quinitofos | 1776-83-6 | ##### | NA 149 | Insecticide | Organophosphorus |
| Quinothion | 22439-40-3 | ##### | NA 140 | Insecticide | Organophosphorus |
| Ronnel | 299-84-3 | 58301 | 517 | Insecticide | Organophosphorus |
| S,S,S-tributyl | 78-48-8 | 74801 | 190 | Defoliant, Plant | Organophosphorus |
| phosphorotrithioate | | | | Growth | |
| | | | | Regulator | |
| S-(1,1-Dimethylethyl) O-ethyl | 83318-76-7 | ##### | NA 104 | Insecticide | Organophosphorus |
| ethylphosphonothioate | | | | | |
| S-(4-(1,1- | 329-21-5 | ##### | NA 143 | Insecticide | Organophosphorus |
| dimethylethyl)phenyl) O-ethyl | | | | | |
| ethylphosphonodithioate | | | | | |
| Sophamide | 37032-15-8 | ##### | NA 144 | Insecticide | Organophosphorus |
| Sulfotep | 3689-24-5 | 79501 | 558 | Insecticide | Organophosphorus |
| Sulfotep, other related | NA 1111 | NA 164 | 90558 | Insecticide | Organophosphorus |
| Sulprofos | 35400-43-2 | ##### | 2006 | Insecticide | Organophosphorus |
| Sulprofos oxon | 38527-90-1 | ##### | NA 125 | Breakdown | Organophosphorus |
| | | | | product | |
| Temephos | 3383-96-8 | 59001 | 1 | Insecticide | Organophosphorus |
| Temephos sulfoxide | 17210-55-8 | 59002 | NA 483 | Breakdown | Organophosphorus |
| | | | | product | |
| TEPP | 107-49-3 | 79601 | 577 | Insecticide | Organophosphorus |
| Terbufos | 13071-79-9 | ##### | 2925 | Insecticide, | Organophosphorus |
| | | | | Nematicide | |
| Terbufos sulfone | 56070-16-7 | ##### | NA 140 | Breakdown | Organophosphorus |
| | | | | product | |
| Terbufos sulfoxide | 10548-10-4 | ##### | NA 140 | Breakdown | Organophosphorus |
| | | | | product | |
| Tetrachlorvinphos | 22248-79-9 | 83702 | 305 | Insecticide | Organophosphorus |
| Tetrachlorvinphos | 961-11-5 | 83701 | NA 34 | Insecticide | Organophosphorus |
| Thiometon | 640-15-3 | ##### | NA 149 | Insecticide | Organophosphorus |
| Thionazin | 297-97-2 | 32401 | 2939 | Insecticide | Organophosphorus |
| Thionazin, O-analog | 7359-55-9 | 32402 | NA 297 | Insecticide, | Organophosphorus |
| | | | | Breakdown | |
| | | | | product | |
| Tolclofos-methyl | 57018-04-9 | ##### | NA 106 | Fungicide | Organophosphorus |
| Triazophos | 24017-47-8 | ##### | 3543 | Insecticide | Organophosphorus |
| Trichlorfon | 52-68-6 | 57901 | 88 | Insecticide | Organophosphorus |
| Trichloronat | 327-98-0 | ##### | 5001 | Insecticide | Organophosphorus |
| Vamidothion | 2275-23-2 | ##### | 3544 | Insecticide | Organophosphorus |
| 3-iodo-2-propynyl butyl | 55406-53-6 | ##### | 1938 | Fungicide, | Other Carbamate |
| carbamate | | | | Wood | |
| | | | | Preservative | |
| 6-Methyl-2-propyl-4- | 2532-49-2 | ##### | NA 157 | N/A | Other Carbamate |
| pyrimidinyl dimethylcarbamate | | | | | |
| Alanycarb | 83130-01-2 | NA 175 | NA 47 | Insecticide | Other Carbamate |
| Asulam | 3337-71-1 | ##### | 5076 | Herbicide | Other Carbamate |
| Asulam, sodium salt | 2302-17-2 | ##### | 1746 | Herbicide | Other Carbamate |
| Benfuracarb | 82560-54-1 | ##### | NA 48 | Insecticide | Other Carbamate |
| Chlorbufam | 1967-16-4 | ##### | NA 157 | Herbicide | Other Carbamate |
| Chlorprocarb | 23121-99-5 | ##### | NA 136 | Herbicide | Other Carbamate |
| Chlorpropham | 101-21-3 | 18301 | 141 | Herbicide, | Other Carbamate |
| | | | | Plant Growth | |
| | | | | Regulator | |
| Diethofencarb | 87130-20-9 | NA 184 | NA 183 | Fungicide | Other Carbamate |
| Dimetan | 122-15-6 | ##### | NA 112 | Insecticide | Other Carbamate |
| Dimetilan | 644-64-4 | 90101 | NA 844 | Insecticide | Other Carbamate |
| Fenoxycarb | 79127-80-3 | ##### | 2283 | Insecticide, | Other Carbamate |
| | | | | Insect Growth | |
| | | | | Regulator | |
| Fenoxycarb | 72490-01-8 | NA 228 | NA 228 | Insecticide, | Other Carbamate |
| | | | | Insect Growth | |
| | | | | Regulator | |
| Isolan | 119-38-0 | ##### | NA 155 | Insecticide | Other Carbamate |
| Karbutilate | 4849-32-5 | 97401 | 691 | Herbicide | Other Carbamate |
| Mecarbinizid | 27386-64-7 | ##### | NA 157 | N/A | Other Carbamate |
| Nitrilacarb | 29672-19-3 | ##### | NA 129 | Insecticide | Other Carbamate |
| Potassium asulam | 14089-43-1 | ##### | NA 893 | Herbicide | Other Carbamate |
| Propamocarb | 24579-73-5 | ##### | 2147 | Fungicide | Other Carbamate |
| Propamocarb hydrochloride | 25606-41-1 | ##### | 4022 | Fungicide | Other Carbamate |
| Propham | 122-42-9 | 47601 | 339 | Herbicide, | Other Carbamate |
| | | | | Plant Growth | |
| | | | | Regulator | |
| Pyrolan | 87-47-8 | ##### | NA 157 | Insecticide | Other Carbamate |
| Swep | 1918-18-9 | 84601 | 4098 | N/A | Other Carbamate |
| Chlorfenapyr | 122453-73-0 | ##### | 3938 | Insecticide | Pyrazole |
| Pyrazoxyfen | 71561-11-0 | NA 189 | NA 189 | Herbicide | Pyrazole |
| Tebufenpyrad | 119168-77-3 | 90102 | NA 845 | Insecticide | Pyrazole |
| (+−)-cis,trans-Deltamethrin | 120710-23-8 | ##### | NA 120 | Insecticide | Pyrethroid |
| (+−)-cis-Permethrin | 52341-33-0 | ##### | NA 120 | Insecticide | Pyrethroid |
| (S)-Cypermethrin | 66841-24-5 | ##### | 3866 | Insecticide | Pyrethroid |
| 6-chloropiperonyl 2,2- | 70-43-9 | ##### | NA 124 | Insecticide | Pyrethroid |
| dimethyl-3-(2- | | | | | |
| methylpropenyl)cyclopropanecarboxylate | | | | | |
| Acrinathrin | 101007-06-1 | ##### | NA 4 | Insecticide | Pyrethroid |
| Allethrin | 584-79-2 | 4001 | 12 | Insecticide | Pyrethroid |
| Allethrin Coil | NA 1163 | 4002 | NA 163 | Insecticide | Pyrethroid |
| Allethrin, other related | NA 1070 | NA 160 | 90012 | Insecticide | Pyrethroid |
| B-fenvalerate | 66267-77-4 | ##### | NA 903 | Insecticide | Pyrethroid |
| Beta-cyfluthrin | 68359-37-5 | ##### | 3956 | Insecticide | Pyrethroid |
| Bifenthrin | 82657-04-3 | ##### | 2300 | Insecticide | Pyrethroid |
| Bioresmethrin | 28434-01-7 | 97802 | NA 855 | Insecticide | Pyrethroid |
| Cismethrin | 35764-59-1 | 97804 | NA 857 | Insecticide | Pyrethroid |
| Cyclethrin | 97-11-0 | 4052 | NA 69 | N/A | Pyrethroid |
| Cycloprothrin | 63935-38-6 | NA 204 | NA 203 | Insecticide | Pyrethroid |
| Cyfluthrin | 68359-37-5 | ##### | 2223 | Insecticide | Pyrethroid |
| Cyhalothrin | 68085-85-8 | ##### | NA 104 | Insecticide | Pyrethroid |
| Cypermethrin (stereochemistry | 52315-07-8 | ##### | NA 172 | Insecticide | Pyrethroid |
| unspecified) | | | | | |
| Cypermethrin, alpha | 67375-30-8 | ##### | NA 120 | Insecticide | Pyrethroid |
| Cypermethrin, beta | 65731-84-2 | ##### | 2171 | Insecticide | Pyrethroid |
| Cypermethrin, theta | 71697-59-1 | NA 200 | NA 199 | Insecticide | Pyrethroid |
| Cyphenothrin | 39515-40-7 | ##### | 3885 | Insecticide | Pyrethroid |
| D-Allethrin | 42534-61-2 | 4005 | 2293 | Insecticide | Pyrethroid |
| D-allethrin, other related | NA 1151 | NA 168 | 92293 | Insecticide | Pyrethroid |
| D-cis-trans Allethrin | 42534-61-2 | 4005 | NA 163 | Insecticide | Pyrethroid |
| D-trans Allethrin | 28057-48-9 | 4003 | 4038 | Insecticide | Pyrethroid |
| D-trans allethrin, other related | NA 1154 | NA 173 | 94038 | Insecticide | Pyrethroid |
| Deltamethrin | 52918-63-5 | 97805 | 3010 | Insecticide | Pyrethroid |
| Deltamethrin (isomer | 52820-00-5 | ##### | NA 120 | Insecticide | Pyrethroid |
| unspecified) | | | | | |
| Deltamethrin, other related | NA 1153 | NA 169 | 93010 | Insecticide | Pyrethroid |
| Dimethrin | 70-38-2 | 34101 | NA 299 | Insecticide | Pyrethroid |
| Empenthrin [(1R)isomers] | 54406-48-3 | NA 202 | NA 202 | Insecticide | Pyrethroid |
| Esbiothrin | 28434-00-6 | 4004 | 4040 | Insecticide | Pyrethroid |
| Esfenvalerate | 66230-04-4 | ##### | 2321 | Insecticide | Pyrethroid |
| Fenpropathrin | 39515-41-8 | ##### | 2234 | Insecticide | Pyrethroid |
| Fenpropathrin | 64257-84-7 | NA 201 | NA 200 | Insecticide | Pyrethroid |
| Fenvalerate | 51630-58-1 | ##### | 1963 | Insecticide | Pyrethroid |
| Fenvalerate | 51630-58-1 | ##### | NA 173 | Insecticide | Pyrethroid |
| Fenvalerate, other related | NA 1143 | NA 167 | 91963 | Insecticide | Pyrethroid |
| Flucythrinate | 70124-77-5 | ##### | 2168 | Insecticide | Pyrethroid |
| Flumethrin | 69770-45-2 | NA 219 | NA 218 | Insecticide | Pyrethroid |
| Fluvalinate (stereochemistry | 69409-94-5 | ##### | 2195 | Insecticide | Pyrethroid |
| unspecified) | | | | | |
| Furethrin | 17080-02-3 | ##### | NA 150 | Insecticide | Pyrethroid |
| Halfenprox | 111872-58-3 | NA 189 | NA 188 | Insecticide | Pyrethroid |
| Imiprothrin | 72963-72-5 | 4006 | 5327 | Insecticide | Pyrethroid |
| Lambda cyhalothrin | 91465-08-6 | ##### | 2297 | Insecticide | Pyrethroid |
| Permethrin | 52645-53-1 | ##### | 2008 | Insecticide | Pyrethroid |
| Permethrin, other related | NA 1146 | NA 168 | 92008 | Insecticide | Pyrethroid |
| Phenothrin | 26002-80-2 | 69005 | 2093 | Insecticide | Pyrethroid |
| Phenothrin, other related | NA 1147 | NA 168 | 92093 | Insecticide | Pyrethroid |
| Prallethrin | 23031-36-9 | ##### | 3985 | Insecticide | Pyrethroid |
| Pyrethrins and pyrethroids, | 68648-44-2 | 69007 | NA 611 | Insecticide | Pyrethroid |
| manufg. Residues | | | | | |
| Resmethrin | 10453-86-8 | 97801 | 2119 | Insecticide | Pyrethroid |
| Resmethrin, other related | NA 1148 | NA 168 | 92119 | Insecticide | Pyrethroid |
| S-Bioallethrin | 28434-00-6 | 4004 | 4039 | Insecticide | Pyrethroid |
| Tau-fluvalinate | 102851-06-9 | NA 201 | NA 200 | Insecticide | Pyrethroid |
| Tefluthrin | 79538-32-2 | ##### | 3839 | Insecticide | Pyrethroid |
| Tetramethrin | 7696-12-0 | 69003 | 1695 | Insecticide | Pyrethroid |
| Tetramethrin, other related | NA 1137 | NA 167 | 91695 | Insecticide | Pyrethroid |
| Tralomethrin | 66841-25-6 | ##### | 2329 | Insecticide | Pyrethroid |
| Transfluthrin | 118712-89-3 | NA 206 | NA 205 | Insecticide | Pyrethroid |
| (Poly-N-acetyl--D- | 9012-76-4 | ##### | 2792 | Plant Growth | Animal derived |
| glucosamine)-protein | | | | Regulator, | |
| | | | | Fungicide | |
| Ammoniated caseinate | 9005-42-9 | NA 539 | 3579 | N/A | Animal derived |
| Androsterone | 53-41-8 | ##### | NA 101 | Deer Repellent | Animal derived |
| Animal gland extracts | NA 63 | NA 118 | 1345 | Rodenticide | Animal derived |
| Animal glue | NA 792 | NA 147 | 3581 | Rodenticide, | Animal derived |
| | | | | Bird Repellent | |
| Beef fat | NA 637 | NA 117 | 3585 | Bait | Animal derived |
| Bone meal | NA 584 | NA 106 | 3589 | N/A | Animal derived |
| Bone oil | 8001-85-2 | 10801 | 78 | Dog and Cat | Animal derived |
| | | | | Repellent | |
| Calcium salts of casein and soy | NA 665 | NA 123 | 1252 | N/A | Animal derived |
| Casein | 9000-71-9 | NA 152 | 1075 | N/A | Animal derived |
| Cat food | NA 796 | NA 147 | 3602 | Bait | Animal derived |
| Cheese | NA 222 | NA 429 | 3604 | Bait | Animal derived |
| Chitin | 1398-61-4 | ##### | NA 109 | Nematicide | Animal derived |
| Clam shells | NA 797 | NA 147 | 3611 | N/A | Animal derived |
| Cod liver oil | 8001-69-2 | 31609 | 2250 | N/A | Animal derived |
| Dried blood | 68911-49-9 | 611 | 1315 | Dog and Cat | Animal derived |
| | | | | Repellent | |
| Dry milk solids | NA 149 | NA 284 | 1976 | N/A | Animal derived |
| Egg shells | NA 141 | NA 275 | 3649 | N/A | Animal derived |
| Eggs | NA 802 | NA 148 | 3648 | N/A | Animal derived |
| Elanco empty gelatin capsules | NA 142 | NA 276 | 2543 | N/A | Animal derived |
| Fish meal | NA 123 | NA 237 | 2571 | N/A | Animal derived |
| Fish oil | 8016-13-5 | ##### | 3658 | Adjuvant, Deer | Animal derived |
| | | | | Repellent, Dog | |
| | | | | and Cat | |
| | | | | Repellent | |
| Game repellent | NA 1490 | NA 221 | NA 221 | Deer Repellent | Animal derived |
| Gardol | 137-16-6 | 174 | 3210 | Dog and Cat | Animal derived |
| | | | | Repellent | |
| Gelatin | 9000-70-8 | NA 222 | 3660 | Insecticide | Animal derived |
| Gelatin capsules, R. P. Scherer | NA 574 | NA 104 | 2577 | N/A | Animal derived |
| Hydrolyzed proteins | NA 1355 | NA 192 | NA 192 | Bait | Animal derived |
| Jaguar | NA 76 | NA 147 | 2146 | Deer Repellent | Animal derived |
| Lard | 61789-99-9 | NA 143 | 3675 | N/A | Animal derived |
| Lion waste | NA 571 | NA 103 | 1301 | Deer Repellent | Animal derived |
| Meat | NA 53 | NA 100 | 3681 | Bait | Animal derived |
| Meat meal | NA 570 | ##### | 3682 | Bait, Deer | Animal derived |
| | | | | Repellent, Dog | |
| | | | | and Cat | |
| | | | | Repellent | |
| Meat scraps | NA 54 | NA 101 | 3683 | Bait | Animal derived |
| Meat stock, Hormel #6 | NA 618 | NA 112 | 2670 | Bait | Animal derived |
| Milk | 8049-98-7 | NA 64 | 3564 | N/A | Animal derived |
| Nutria meat | NA 24 | NA 36 | 3702 | Bait | Animal derived |
| Oil of mink | NA 10 | NA14 | 1961 | N/A | Animal derived |
| Oyster shells | NA 808 | NA 149 | 3714 | N/A | Animal derived |
| Pig tasties | NA 594 | NA 107 | 3730 | N/A | Animal derived |
| Powdered gelatin | NA 760 | NA 137 | 2827 | N/A | Animal derived |
| Putrescent whole egg solids | 51609-52-0 | ##### | 1935 | Insecticide, | Animal derived |
| | | | | Deer Repellent | |
| Shellac | 9000-59-3 | NA 109 | 3378 | N/A | Animal derived |
| Silkworm pupae | NA 464 | NA 828 | 2855 | N/A | Animal derived |
| Sodium caseinate | 9005-46-3 | NA 836 | 3389 | N/A | Animal derived |
| Sperm oil | 8002-24-2 | 99701 | NA 868 | N/A | Animal derived |
| Whey | NA 560 | NA 101 | 3825 | N/A | Animal derived |
| Wool | NA 566 | NA 102 | 3826 | N/A | Animal derived |
| (−)-.alpha.-Cubebene | 17699-14-8 | ##### | NA 130 | Insecticide | Botanical |
| (7S)-Hydroprene | 65733-18-8 | ##### | NA 107 | Insect Growth | Botanical |
| | | | | Regulator | |
| 1-naphthaleneacetamide | 86-86-2 | 56001 | 422 | Plant Growth | Botanical, |
| (NAD) | | | | Regulator | Naphthalene acetic |
| | | | | | acid derivati |
| 2-methoxy-5-nitro phenol, | 67233-85-6 | ##### | 5117 | Plant Growth | Botanical |
| sodium salt | | | | Regulator | |
| 2-Naphthyloxyacetamide | NA 1401 | NA 197 | NA 196 | Plant Growth | Naphthalene acetic |
| | | | | Regulator, | acid derivati, |
| | | | | Herbicide | Botanical |
| 3,7-dimethyl-6-octanol | 106-22-9 | NA 129 | 2140 | Insect | Botanical |
| | | | | Repellent | |
| 3,7-dimethyl-6-octen-1-ol | 150-84-5 | NA 307 | 2522 | Insect | Botanical |
| acetate | | | | Repellent | |
| Acacia | 2591766 | NA 696 | 2346 | N/A | Botanical |
| Alfalfa | NA 635 | NA 116 | 3569 | N/A | Botanical |
| Alfalfa meal | NA 393 | NA 714 | 3570 | N/A | Botanical |
| Algin | 9005-38-3 | NA 715 | 3571 | N/A | Botanical |
| Almond hulls | NA 280 | NA 547 | 3577 | N/A | Botanical |
| Almond shells | NA 636 | NA 117 | 3578 | N/A | Botanical |
| Alpha-ionone | 127-41-3 | ##### | 1518 | Dog and Cat | Botanical |
| | | | | Repellent | |
| Alpha-pinene polymer | 31393-98-3 | NA 153 | 3996 | Insecticide | Botanical |
| Amycol potato alpha starch | NA 272 | NA 527 | 2387 | N/A | Botanical |
| Apple pomace | NA 262 | NA 517 | 3582 | N/A | Botanical |
| Avermectin | 71751-41-2 | ##### | 2254 | Insecticide | Botanical |
| Avermectin B1 | 65195-55-3 | ##### | NA 165 | Insecticide | Botanical |
| Avermectin, other related | NA 1156 | NA 174 | 92254 | Insecticide | Botanical |
| Azadirachtin | 11141-17-6 | ##### | 2328 | Insecticide, | Botanical |
| | | | | Nematicide | |
| Azadirachtin B | 95507-03-2 | ##### | NA 989 | Insecticide | Botanical |
| Balsam peru | NA 660 | NA 122 | 1146 | N/A | Botanical |
| Barley straw | NA 873 | NA 160 | 5138 | N/A | Botanical |
| Beeswax (yellow and white) | NA 245 | NA 494 | 3586 | N/A | Botanical |
| Beet powder | NA 246 | NA 495 | 3587 | N/A | Botanical |
| Beta-caryophyllene | 87-44-5 | NA 439 | 2188 | N/A | Botanical |
| Beta-pinene polymer | 68240-09-5 | NA 153 | 3998 | Insecticide | Botanical |
| Bran | NA 638 | NA 117 | 3590 | N/A | Botanical |
| Bread crumbs | NA 793 | NA 147 | 3591 | Bait | Botanical |
| Buffalo gourd root powder | NA 238 | NA 475 | 3928 | N/A | Botanical |
| Calf's Milk Replacer | NA 1320 | NA 177 | 5741 | N/A | Botanical |
| Canary seed | NA 583 | NA 105 | 3597 | Bait | Botanical |
| Capsicum oleoresin | 404-86-4 | 70701 | 470 | Insecticide | Botanical |
| Cardboard | NA 795 | NA 147 | 3599 | N/A | Botanical |
| Carrageenan | 2591943 | NA 437 | 3600 | Adjuvant | Botanical |
| Carrots | NA 226 | NA 438 | 3601 | N/A | Botanical |
| Chlorophyllin | 11006-34-1 | NA 191 | NA 190 | Fungicide, | Inorganic-Copper, |
| | | | | Microbiocide | Botanical |
| Cinerin I | 25402-06-6 | 69009 | NA 612 | Insecticide | Botanical |
| Cinerin II | 121-20-0 | 69010 | NA 613 | Insecticide | Botanical |
| Cinnamaldehyde | 104-55-2 | 40506 | 2277 | Dog and Cat | Botanical |
| | | | | Repellent, | |
| | | | | Fungicide, Dog | |
| | | | | and Cat | |
| | | | | Repellent | |
| Cinnamon | NA 217 | ##### | 3607 | Insecticide | Botanical |
| cis-Zeatin | NA 1342 | NA 191 | NA 190 | Plant Growth | Botanical |
| | | | | Regulator | |
| Citronellal | 106-23-0 | NA 223 | NA 223 | Insect | Botanical |
| | | | | Repellent | |
| Citronellol | 106-22-9 | NA 306 | 2139 | Insecticide | Botanical |
| Citrus extract | NA 1457 | NA 216 | NA 215 | N/A | Botanical |
| Citrus meal | NA 214 | NA 407 | 3608 | N/A | Botanical |
| Citrus pectin | NA 215 | NA 408 | 3609 | N/A | Botanical |
| Citrus pulp | NA 216 | NA 409 | 3610 | N/A | Botanical |
| Clarified hydrophobic extract | 8002-65-1 | 25006 | 3979 | Insecticide | Botanical |
| of neem oil | | | | | |
| Clarified hydrophobic neem oil | 8002-65-1 | 25007 | NA 172 | Insecticide | Botanical |
| Cloves, Crushed | NA 962 | ##### | 2333 | Insecticide | Botanical |
| Coco shell flour | NA 200 | NA 392 | 3617 | N/A | Botanical |
| Cocoa | NA 207 | NA 398 | 3613 | N/A | Botanical |
| Cocoa shells | NA 640 | NA 117 | 3614 | N/A | Botanical |
| Coffee grounds | NA 581 | NA 105 | 3619 | N/A | Botanical |
| Components of etheric oils of | NA 1360 | NA 193 | NA 192 | Insecticide | Botanical |
| plant origin | | | | | |
| Cork | NA 798 | NA 147 | 3622 | N/A | Botanical |
| Corn | NA 879 | NA 169 | 5144 | Bait | Botanical |
| Corn cobs | NA 193 | NA 383 | 3624 | N/A | Botanical |
| Corn flour | NA 799 | NA 147 | 3625 | N/A | Botanical |
| Corn gluten meal | 66071-96-3 | ##### | 2481 | Herbicide | Botanical |
| Corn product, hydrolyzed | NA 710 | NA 130 | 2322 | Herbicide | Botanical |
| Cotton | NA 188 | NA 376 | 3629 | N/A | Botanical |
| Cotton fibre cord | NA 189 | NA 377 | 2482 | N/A | Botanical |
| Cottonseed flour | 12751-36-9 | NA 129 | 2078 | N/A | Botanical |
| Cottonseed meal | NA 190 | NA 378 | 3630 | N/A | Botanical |
| Cracked oats | NA 801 | NA 148 | 3632 | Bait | Botanical |
| Cracked wheat | NA 192 | NA 380 | 3633 | Bait | Botanical |
| Creosote, wood | 8021-39-4 | 25002 | NA 202 | Wood | Botanical |
| | | | | Preservative | |
| Cube extracts | NA 958 | 71004 | 756 | Insecticide | Botanical |
| Cytokinin | 525-79-1 | ##### | 2082 | Plant Growth | Botanical |
| | | | | Regulator | |
| Cytokinin (as kinetin) | 525-79-1 | ##### | NA 172 | Plant Growth | Botanical |
| | | | | Regulator | |
| Daphne oil | NA 1354 | NA 192 | NA 192 | Insect | Botanical |
| | | | | Repellent | |
| Derris resins other than | NA 1260 | 71001 | NA 172 | Insecticide | Botanical |
| rotenone | | | | | |
| Desmodium | NA 1560 | NA 227 | NA 227 | Insecticide | Botanical |
| Digitalis | 71-63-6 | 97002 | NA 850 | N/A | Botanical |
| Digitalis | 1339-93-1 | 97002 | NA 851 | N/A | Botanical |
| Dihydroazadirachtin | 108189-58-8 | ##### | 3994 | Insecticide, | Botanical |
| | | | | Nematicide | |
| DMN | 571-58-4 | 55802 | 5067 | Plant Growth | Botanical |
| | | | | Regulator | |
| Douglas fir bark, ground | NA 156 | NA 292 | 3647 | N/A | Botanical |
| Emamectin, benzoate | 137512-74-4 | ##### | 4020 | Insecticide | Botanical |
| Ethylene glycol ether of pinene | 53404-49-2 | 42101 | NA 363 | Insecticide | Botanical |
| Eugenol | 97-53-0 | ##### | 2095 | Pheromone, | Botanical |
| | | | | Fragrance, Dog | |
| | | | | and Cat | |
| | | | | Repellent, | |
| | | | | Insecticide, | |
| | | | | Insect | |
| | | | | Repellent | |
| Fenugreek | NA 121 | NA 234 | 3657 | N/A | Botanical |
| Flour | NA 120 | NA 232 | 3659 | N/A | Botanical |
| Flowers of Chamomile | NA 1267 | ##### | NA 173 | Insecticide | Botanical |
| (Matricaria chamomilla and/or | | | | | |
| Anthemidis nobilis) | | | | | |
| Folic acid | 59-30-3 | NA 191 | NA 190 | Plant Growth | Botanical |
| | | | | Regulator | |
| Garlic | 8000-78-0 | ##### | 2213 | Insecticide | Botanical |
| Geraniol | 106-24-1 | ##### | 309 | Pheromone, | Botanical |
| | | | | Fragrance, | |
| | | | | Insecticide, | |
| | | | | Insect | |
| | | | | Repellent, Dog | |
| | | | | and Cat | |
| | | | | Repellent | |
| Gibberellic acid, 2-butoxyethyl | 6550-86-3 | 43803 | NA 371 | Plant Growth | Botanical |
| ester | | | | Regulator | |
| Gibberellin A4 mixt. With | 8030-53-3 | ##### | NA 952 | Plant Growth | Botanical |
| gibberellin A7 | | | | Regulator | |
| Gibberellins | 77-06-5 | 43801 | 310 | Plant Growth | Botanical |
| | | | | Regulator | |
| Gibberellins, potassium salt | 125-67-7 | 43802 | 771 | Plant Growth | Botanical |
| | | | | Regulator | |
| Grape pomace | NA 880 | NA 169 | 5145 | N/A | Botanical |
| Griseofulvin | 126-07-8 | ##### | NA 150 | N/A | Botanical |
| Ground corn cobs | NA 732 | NA 133 | 2583 | N/A | Botanical |
| Ground oats | NA 804 | NA 148 | 3664 | Bait | Botanical |
| Ground rice hulls | NA 108 | NA 212 | 2584 | N/A | Botanical |
| Ground sesame plant | NA 109 | ##### | 2585 | Insecticide, | Botanical |
| | | | | Nematicide | |
| Guar gum | 9000-30-0 | NA 103 | 3666 | Adjuvant | Botanical |
| Gum resins | 9000-75-3 | 99401 | NA 866 | Adjuvant | Botanical |
| Gum Thus | 2244967 | 84502 | NA 834 | Adjuvant | Botanical |
| Gum tragacanth | 9000-65-1 | NA 213 | 3218 | Adjuvant | Botanical |
| Hearts of corn flour | NA 573 | NA 104 | 3667 | N/A | Botanical |
| Hellebore alkaloids | 1399-70-8 | 2001 | NA 65 | N/A | Botanical |
| Humic acid | 1415-93-6 | NA 134 | 2597 | N/A | Botanical |
| Hydroprene | 41096-46-2 | ##### | 2244 | Insect Growth | Botanical |
| | | | | Regulator | |
| Indole | 120-72-9 | 25000 | 2614 | Insecticide, | Botanical |
| | | | | Insect | |
| | | | | Repellent, Dog | |
| | | | | and Cat | |
| | | | | Repellent | |
| Indole-3-acetic acid | 87-51-4 | ##### | NA 106 | Plant Growth | Botanical |
| | | | | Regulator | |
| Jasmolin I | 4466-14-2 | 69011 | NA 614 | Insecticide | Botanical |
| Jasmolin II | 1172-63-0 | 69012 | NA 615 | Insecticide | Botanical |
| Juniper tar oil | 2231543 | 67203 | NA 575 | Insecticide | Botanical |
| Juniperus communis ext. | 84603-69-0 | 67212 | NA 582 | Insecticide | Botanical |
| Larkspur alkaloid | 41710-20-7 | 2202 | NA 66 | N/A | Botanical |
| Leaves of Eucalyptus | NA 1277 | ##### | NA 174 | Insecticide | Botanical |
| Leaves of Pennyroyal (Mentha | NA 1278 | ##### | NA 174 | Insecticide | Botanical |
| pulegium) | | | | | |
| Lignin | NA 73 | NA 140 | 2640 | N/A | Botanical |
| Lilacin | 118-34-3 | 67006 | NA 573 | N/A | Botanical |
| Linalool | 78-70-6 | ##### | 2239 | Insecticide, | Botanical |
| | | | | Insect | |
| | | | | Repellent, | |
| | | | | Fragrance | |
| Locust bean gum | NA 738 | NA 134 | 2647 | Adjuvant | Botanical |
| Medicated feed | NA 644 | NA 118 | 3685 | N/A | Botanical |
| Millet seed | NA 38 | NA 65 | 3692 | Bait | Botanical |
| Mint Herbs | NA 1280 | ##### | NA 174 | Insecticide | Botanical |
| Montok pepper | NA 851 | NA 158 | 5012 | Insecticide | Botanical |
| Myrica cerifera, extract | 84929-34-0 | ##### | NA 123 | N/A | Botanical |
| N6-Benzyladenine, mixt. with | 53663-71-1 | ##### | NA 953 | Plant Growth | Botanical |
| Gibberellins A4 and A7 | | | | Regulator | |
| NAA | 86-87-3 | 56002 | 423 | Plant Growth | Naphthalene acetic |
| | | | | Regulator | acid derivati, |
| | | | | | Botanical |
| NAA, ethyl ester | 2122-70-5 | 56008 | 749 | Plant Growth | Naphthalene acetic |
| | | | | Regulator | acid derivati, |
| | | | | | Botanical |
| Nicotine | 54-11-5 | 56702 | 75 | Insecticide | Botanical |
| Nicotine sulfate | 65-30-5 | 56703 | 430 | Insecticide | Botanical |
| NOA | 120-23-0 | 55601 | 432 | Plant Growth | Naphthalene acetic |
| | | | | Regulator | acid derivati, |
| | | | | | Botanical |
| Nonanoic acid | 112-05-0 | ##### | 2739 | Herbicide, | Botanical |
| | | | | Fungicide | |
| Oatmeal | NA 806 | NA 149 | 3704 | Bait | Botanical |
| Oats | NA 16 | NA 26 | 3705 | Bait | Botanical |
| Oil of cedarwood | 8000-27-9 | 40505 | 1011 | Insecticide, | Botanical |
| | | | | Fungicide | |
| Oil of hardwood | NA 675 | 67201 | 1639 | Microbiocide | Botanical |
| Oil of pine tar | 2227495 | 67002 | 2246 | Insecticide, | Botanical |
| | | | | Microbiocide | |
| Oils, cedarwood, Texan | 68990-83-0 | 11524 | NA 118 | Insecticide, | Botanical |
| | | | | Fungicide | |
| Onion extract | NA 1349 | NA 192 | NA 191 | N/A | Botanical |
| Onions | NA 807 | NA 149 | 3711 | N/A | Botanical |
| Orange pulp | NA 9 | NA 13 | 3712 | N/A | Botanical |
| p-Menthane-3,8-diol | 42822-86-6 | 11550 | 5255 | Insect | Botanical |
| | | | | Repellent | |
| Paloja | 9000-28-6 | ##### | NA 880 | N/A | Botanical |
| Papain | 9001-73-4 | NA 149 | 3717 | Rodenticide | Botanical |
| Paper | NA 598 | NA 107 | 3718 | N/A | Botanical |
| Paprika | NA 383 | NA 692 | 3719 | N/A | Botanical |
| Peanut butter | NA 373 | NA 681 | 2735 | Bait | Botanical |
| Peanut shells | NA 375 | NA 683 | 3723 | N/A | Botanical |
| Peanuts | NA 374 | NA 682 | 3533 | Bait | Botanical |
| Peat moss | NA 809 | NA 149 | 3724 | N/A | Botanical |
| Pecan shell flour | NA 376 | NA 684 | 3725 | N/A | Botanical |
| Pectin | 9000-69-5 | NA 118 | 3726 | N/A | Botanical |
| Pepper | NA 1348 | NA 192 | NA 191 | Insecticide, | Botanical |
| | | | | Deer Repellent | |
| Peppermint | NA 365 | NA 668 | 2332 | Insecticide, | Botanical |
| | | | | Fragrance | |
| Phenylethyl propionate | 122-70-3 | ##### | 2094 | Fragrance, | Botanical |
| | | | | Pheromone, | |
| | | | | Insecticide, | |
| | | | | Insect | |
| | | | | Repellent, Dog | |
| | | | | and Cat | |
| | | | | Repellent | |
| Pine oil | 2227495 | 67002 | 485 | Insecticide, | Botanical |
| | | | | Microbiocide | |
| Pine soap | NA 1032 | NA 127 | 1993 | Insecticide, | Botanical |
| | | | | Microbiocide | |
| Pine tar | 8011-48-1 | 67204 | 1574 | Insecticide, | Botanical |
| | | | | Microbiocide | |
| Pine tar oil | 91995-59-4 | 67205 | NA 576 | Insecticide, | Botanical |
| | | | | Microbiocide | |
| Pinene | 80-56-8 | 67004 | 2187 | Insecticide | Botanical |
| Pinene | 1330-16-1 | 67001 | NA 572 | Insecticide | Botanical |
| Plant carbolineums | NA 1454 | NA 215 | NA 215 | Insecticide, | Botanical |
| | | | | Fungicide | |
| Plant extract (derived from | NA 917 | ##### | 5453 | Nematicide | Botanical |
| Quercus falcata, Opuntia | | | | | |
| lindheimeri, Rhus aromatica, | | | | | |
| and Rhizophoria mangle | | | | | |
| tissues) | | | | | |
| Polymerized pinene | NA 399 | NA 722 | 2043 | Insecticide | Botanical |
| Potatoes | NA 436 | NA 786 | 3772 | N/A | Botanical |
| Poultry feed | NA 437 | NA 787 | 3773 | N/A | Botanical |
| Pyrethrin coils | NA 1165 | 69004 | NA 164 | Insecticide | Botanical |
| Pyrethrin I | 121-21-1 | NA 213 | NA 213 | Insecticide | Botanical |
| Pyrethrin II | 121-29-9 | 69006 | NA 610 | Insecticide | Botanical |
| Pyrethrins | 8003-34-7 | 69001 | 510 | Insecticide | Botanical |
| Pyrethrins, other related | NA 1108 | NA 164 | 90510 | Insecticide | Botanical |
| Pyrethrum narc | NA 1000 | 69000 | 1461 | Insecticide | Botanical |
| Pyrethrum powder other than | 8003-34-7 | 69002 | NA 164 | Insecticide | Botanical |
| pyrethrins | | | | | |
| Quassia | NA 450 | NA 806 | 3778 | Insecticide | Botanical |
| Quillaja | 68990-67-0 | NA 150 | 3779 | N/A | Botanical |
| Raisins | NA 647 | NA 119 | 3780 | Bait | Botanical |
| Red dog flour | NA 452 | NA 811 | 3782 | N/A | Botanical |
| Red pepper | NA 107 | 70703 | 1094 | Dog and Cat | Botanical |
| | | | | Repellent | |
| Red squill glycoside | 507-60-8 | 70801 | 514 | Rodenticide | Botanical |
| Repellants (by smell) of animal | NA 1373 | NA 194 | NA 193 | Dog and Cat | Botanical |
| or plant origin | | | | Repellent, Deer | |
| | | | | Repellent | |
| Resins, oleo-, capsicum | 8023-77-6 | 70704 | NA 665 | Insecticide | Botanical |
| Rice | NA 812 | NA 150 | 3783 | N/A | Botanical |
| Rice hulls | NA 813 | NA 150 | 3784 | N/A | Botanical |
| Rosemary | NA 453 | ##### | 2331 | N/A | Botanical |
| Rosin | 9014-63-5 | NA 812 | 3785 | N/A | Botanical |
| Rosin oil | 8002-16-2 | 40508 | NA 341 | N/A | Botanical |
| Rosin, partially dimerized | NA 454 | NA 813 | 3786 | N/A | Botanical |
| Rosin, partially hydrogenated | 65997-06-0 | NA 150 | 3787 | N/A | Botanical |
| Rotenone | 83-79-4 | 71003 | 518 | Insecticide | Botanical |
| Rotenone, other related | NA 1109 | NA 164 | 90518 | Insecticide | Botanical |
| Rubber | 2594048 | NA 814 | 3788 | N/A | Botanical |
| Ryanodine | NA 1305 | 71502 | NA 175 | Insecticide | Botanical |
| Ryanodine alkaloid | 15662-33-6 | 71501 | 520 | Insecticide | Botanical |
| Rye flour | NA 648 | NA 119 | 3789 | N/A | Botanical |
| Sabadilla alkaloids | 2245180 | 2201 | 521 | Insecticide | Botanical |
| Safrole | 94-59-7 | 97901 | 3376 | N/A | Botanical |
| Saponin | 8047-15-2 | 97004 | 5033 | N/A | Botanical |
| Sawdust | NA 1306 | ##### | 1316 | Adjuvant | Botanical |
| Scratch feed | NA 649 | NA 119 | 3792 | N/A | Botanical |
| Sea-algae extract | NA 1352 | NA 192 | NA 191 | Plant Growth | Botanical |
| | | | | Regulator, | |
| | | | | Adjuvant | |
| Seaweed | NA 814 | NA 150 | 3793 | N/A | Botanical |
| Sodium 2-nitrophenoxide | 824-39-5 | ##### | 5118 | Plant Growth | Botanical |
| | | | | Regulator | |
| Sodium 4-nitrophenoxide | 824-78-2 | ##### | 5119 | Plant Growth | Botanical |
| | | | | Regulator | |
| Sorghum, grain | NA 478 | NA 882 | 3904 | N/A | Botanical |
| Soy flour | NA 482 | NA 886 | 3809 | N/A | Botanical |
| Soy protein | NA 483 | NA 887 | 3810 | N/A | Botanical |
| Soybean extract | NA 1353 | NA 192 | NA 191 | N/A | Botanical |
| Soybean hulls | NA 481 | NA 885 | 3807 | N/A | Botanical |
| Soybean meal | NA 817 | NA 150 | 3808 | N/A | Botanical |
| Squalane | 111-01-3 | 45503 | 2887 | N/A | Botanical |
| Sugarbeet meal | NA 499 | NA 910 | 3813 | N/A | Botanical |
| Sunflower seeds | NA 650 | NA 119 | 3815 | Bait | Botanical |
| Synthetic vegetable gums | 9004-64-2 | NA 921 | 1835 | Adjuvant | Botanical |
| Tall oil | 8002-26-4 | 67211 | 2912 | Adjuvant | Botanical |
| Tall oil acids | 61790-12-3 | NA 124 | 1389 | Adjuvant | Botanical |
| Tar oils, from distillation of | 8002-29-7 | 67202 | NA 574 | Insecticide, | Botanical |
| wood tar | | | | Herbicide | |
| Thuringiensin, calcium | 23526-02-5 | 6404 | 2941 | Insecticide | Botanical |
| technical | | | | | |
| Thyme | NA 527 | ##### | 2330 | Insect | Botanical |
| | | | | Repellent, | |
| | | | | Insecticide | |
| Tobacco | NA 1011 | NA 959 | 3932 | Insecticide | Botanical |
| Tobacco dust | 8037-19-2 | 56704 | NA 469 | Insecticide | Botanical |
| Trimethoxybenzene | 135-77-3 | 40515 | 5064 | Insecticide, | Botanical |
| | | | | Insect | |
| | | | | Repellent, Dog | |
| | | | | and Cat | |
| | | | | Repellent | |
| Tung oil | NA 547 | NA 997 | 3821 | N/A | Botanical |
| Verbenone | 1196-01-6 | ##### | NA 108 | Insect | Botanical |
| | | | | Repellent | |
| Walnut flour | NA 555 | NA 101 | 3823 | N/A | Botanical |
| Walnut shells, ground | NA 556 | NA 101 | 2986 | N/A | Botanical |
| Wheat | NA 819 | NA 151 | 3824 | N/A | Botanical |
| Wheat flour | NA 674 | NA 125 | 1577 | N/A | Botanical |
| White pepper | NA 1311 | NA 178 | NA 177 | Insecticide | Botanical |
| Wood powder | NA 565 | NA 102 | 1463 | N/A | Botanical |
| Wood rosin | 2245031 | 67208 | NA 579 | N/A | Botanical |
| Wood tar | 91722-33-7 | 67206 | NA 577 | Insecticide | Botanical |
| Xanthan gum | 11138-66-2 | NA 102 | 2272 | Adjuvant | Botanical |
| Agrobacterium radiobacter var | NA 1435 | NA 211 | NA 210 | Fungicide | Microbial |
| radiobacter | | | | | |
| Agrobacterium radiobacter | NA 693 | NA 127 | 1984 | Fungicide | Microbial |
| Agrobacterium radiobacter | NA 925 | ##### | NA 167 | Fungicide | Microbial |
| (strain K84) | | | | | |
| Agrobacterium radiobacter | NA 921 | 6474 | 5477 | Fungicide | Microbial |
| strain K1026 | | | | | |
| Agrobacterium vitis | NA 1536 | NA 224 | NA 224 | Fungicide | Microbial |
| Agrotis segetum granulosis | NA 1391 | NA 196 | NA 195 | Insecticide | Microbial |
| virus | | | | | |
| Ampelomyces quisqualis | NA 270 | NA 525 | 2385 | Fungicide | Microbial |
| Ampelomyces quisqualis | NA 1217 | 21007 | NA 170 | Fungicide | Microbial |
| isolate M-10 | | | | | |
| Anagrapha falcifera multi- | NA 862 | ##### | 5089 | Insecticide | Microbial |
| nuclear polyhedrosis virus | | | | | |
| (AFMNPV) | | | | | |
| Aschersonia aleyrodis | NA 1392 | NA 196 | NA 195 | Insecticide | Microbial |
| Bacillus cereus strain UW85 | NA 1218 | ##### | NA 170 | Plant Growth | Microbial |
| | | | | Regulator | |
| Bacillus cereus, strain BP01 | NA 903 | NA 172 | 5183 | Insecticide | Microbial |
| Bacillus popilliae | NA 257 | NA 509 | 2183 | Fungicide | Microbial |
| Bacillus popilliae and B. lentimorbus | NA 1219 | 54501 | NA 170 | Fungicide | Microbial |
| Bacillus sphaericus | 143447-72-7 | ##### | 2274 | Insecticide | Microbial |
| Bacillus sphaericus, serotype | 143447-72-7 | ##### | 2409 | Insecticide | Microbial |
| H-5A5B, strain 2362 | | | | | |
| Bacillus subtilis GBO3 | NA 855 | ##### | 3945 | Fungicide | Microbial |
| Bacillus subtilis isolate B246 | NA 1538 | NA 225 | NA 224 | Fungicide | Microbial |
| Bacillus subtilis MBI 600 | NA 864 | ##### | 5120 | Fungicide | Microbial |
| Bacillus subtilis var. | NA 1220 | 6480 | NA 170 | Fungicide, | Microbial |
| Amyloliquefaciens Strain | | | | Plant Growth | |
| FZB24 | | | | Regulator | |
| Bacillus thuringiensis (berliner) | 68038-71-1 | 6400 | 86 | Insecticide | Microbial |
| Bacillus thuringiensis | 68038-71-1 | 6426 | 3843 | Insecticide | Microbial |
| (berliner), subsp. Aizawai, GC- | | | | | |
| 91 protein | | | | | |
| Bacillus thuringiensis | 68038-71-1 | 6400 | 3856 | Insecticide | Microbial |
| (berliner), subsp. Aizawai, | | | | | |
| serotype H-7 | | | | | |
| Bacillus thuringiensis | 68038-71-1 | 6401 | 3857 | Insecticide | Microbial |
| (berliner), subsp. Israelensis, | | | | | |
| serotype H-14 | | | | | |
| Bacillus thuringiensis | 68038-71-1 | 6402 | 3970 | Insecticide | Microbial |
| (berliner), subsp. Kurstaki | | | | | |
| strain SA-12 | | | | | |
| Bacillus thuringiensis | 68038-71-1 | 6402 | 3858 | Insecticide | Microbial |
| (berliner), subsp. Kurstaki, | | | | | |
| serotype 3a, 3b | | | | | |
| Bacillus thuringiensis | 68038-71-1 | 6424 | 3859 | Insecticide | Microbial |
| (berliner), subsp. Kurstaki, | | | | | |
| strain EG2348 | | | | | |
| Bacillus thuringiensis | 68038-71-1 | 6423 | 3860 | Insecticide | Microbial |
| (berliner), subsp. Kurstaki, | | | | | |
| strain EG2371 | | | | | |
| Bacillus thuringiensis | 68038-71-1 | 6422 | 3861 | Insecticide | Microbial |
| (berliner), subsp. Kurstaki, | | | | | |
| strain EG2424 | | | | | |
| Bacillus thuringiensis | 68038-71-1 | 6402 | 3862 | Insecticide | Microbial |
| (berliner), subsp. Kurstaki, | | | | | |
| strain SA-11 | | | | | |
| Bacillus thuringiensis | NA 1481 | 6400 | 3863 | Insecticide | Microbial |
| (berliner), subsp. Morrisoni, | | | | | |
| serotype 8a8b | | | | | |
| Bacillus thuringiensis | 68038-71-1 | 6400 | 3864 | Insecticide | Microbial |
| (berliner), subsp. San Diego | | | | | |
| Bacillus thuringiensis | 68038-71-1 | 6404 | 2410 | Insecticide | Microbial |
| darstadiensis | | | | | |
| Bacillus thuringiensis sub. | 68038-71-1 | 6448 | 5051 | Insecticide | Microbial |
| Kurstaki strain EG7673 | | | | | |
| coleopteran active toxin | | | | | |
| Bacillus thuringiensis sub. | 68038-71-1 | 6447 | 5049 | Insecticide | Microbial |
| Kurstaki strain EG7673 | | | | | |
| lepidopteran active toxin | | | | | |
| Bacillus thuringiensis subsp. | NA 1224 | 6403 | NA 170 | Insecticide | Microbial |
| Aizawai | | | | | |
| Bacillus thuringiensis subsp. | 68038-71-1 | 6402 | 5004 | Insecticide | Microbial |
| Kurstaki, genetically | | | | | |
| engineered strain AGRO1 by | | | | | |
| Agrevo | | | | | |
| Bacillus thuringiensis subsp. | 68038-71-1 | 6402 | 5005 | Insecticide | Microbial |
| Kurstaki, genetically | | | | | |
| engineered strain AGRO2 by | | | | | |
| Agrevo | | | | | |
| Bacillus thuringiensis subsp. | NA 1232 | ##### | NA 171 | Insecticide | Microbial |
| San Diego | | | | | |
| Bacillus thuringiensis subsp. | 68038-71-1 | 6405 | 5043 | Insecticide | Microbial |
| Tenebrionis | | | | | |
| Bacillus thuringiensis subspec. | 68038-71-1 | NA 169 | 3972 | Insecticide | Microbial |
| Tenebrionis delta endotoxin | | | | | |
| Bacillus thuringiensis | NA 1234 | 6476 | NA 171 | Insecticide | Microbial |
| subspecies Israelensis strain | | | | | |
| EG2215 | | | | | |
| Bacillus thuringiensis | 68038-71-1 | 6401 | 4018 | Insecticide | Microbial |
| subspecies Israelensis, strain | | | | | |
| IPS-78 | | | | | |
| Bacillus thuringiensis | 68038-71-1 | 6407 | 4024 | Insecticide | Microbial |
| subspecies kurstaki strain BMP | | | | | |
| 123 | | | | | |
| Bacillus thuringiensis | 68038-71-1 | 6402 | 5010 | Insecticide | Microbial |
| subspecies Kurstaki strain HD- | | | | | |
| 1, lepidopteran active toxin | | | | | |
| Bacillus thuringiensis | 68038-71-1 | 6453 | 3988 | Insecticide | Microbial |
| subspecies kurstaki, genetically | | | | | |
| engineered strain EG7841 | | | | | |
| lepidopteran active toxin | | | | | |
| Bacillus thuringiensis var. | NA 1238 | 6445 | NA 171 | Insecticide | Microbial |
| kurstaki delta endotoxin protein | | | | | |
| as produced by the Cry1A(c) | | | | | |
| gene and its controlling | | | | | |
| sequences | | | | | |
| Bacillus thuringiensis var. | 68038-71-1 | 6452 | 3978 | Insecticide | Microbial |
| Kurstaki strain M-200 protein | | | | | |
| toxin | | | | | |
| Bacillus thuringiensis var. | 68038-71-1 | 6402 | 3962 | Insecticide | Microbial |
| Kurstaki, genetically | | | | | |
| engineered strain ECX 9441 | | | | | |
| Bacillus thuringiensis var. | 68038-71-1 | 6459 | 5325 | Insecticide | Microbial |
| Kurstaki, genetically | | | | | |
| engineered strain EG7826 | | | | | |
| Lepidopteran active toxin | | | | | |
| Neodiprion sertifer nuclear | NA 1389 | NA 196 | NA 195 | N/A | Microbial |
| polyhedrosis virus | | | | | |
| Nosema locustae spores | NA 20 | ##### | 2137 | Insecticide | Microbial |
| Nuclear polyhedrosis virus | NA 1451 | NA 215 | NA 214 | Insecticide | Microbial |
| Nuclear polyhedrosis virus of | NA 1430 | NA 213 | NA 213 | N/A | Microbial |
| Helicoverpea zea | | | | | |
| Nuclear polyhedrosis virus of | NA 1510 | NA 222 | NA 222 | Insecticide | Microbial |
| red-headed pine sawfly | | | | | |
| Nystatin (streotomyces | 1400-61-9 | ##### | NA 115 | N/A | Microbial |
| noursei) (withdrawn: 23614-R: | | | | | |
| pm24) | | | | | |
| Paecilomyces fumosoroseus | NA 831 | ##### | 3964 | Insecticide | Microbial |
| apopka strain 97 | | | | | |
| Paecilomyces Lilacinus Strain | NA 1439 | NA 213 | NA 213 | Insecticide | Microbial |
| 251 | | | | | |
| Penicillin G, potassium salt | NA 666 | NA 123 | 1283 | N/A | Microbial |
| Peniophora gigantea | NA 1462 | NA 217 | NA 216 | Fungicide | Microbial |
| Phlebiopsis gigantea | NA 1388 | NA 196 | NA 195 | Fungicide | Microbial |
| Phytophthora palmivora, | NA 860 | ##### | 5078 | Herbicide | Microbial |
| chlamydospores of | | | | | |
| Polyhedral inclusion bodies of | NA 593 | ##### | 2009 | Insecticide | Microbial |
| Douglas fir tussock moth | | | | | |
| nuclear polyhedrosis virus | | | | | |
| Polyhedral inclusion bodies of | NA 1289 | ##### | NA 175 | Insecticide | Microbial |
| gypsy moth nucleopolyhedrosis | | | | | |
| virus | | | | | |
| Polyhedral inclusion bodies of | NA 1290 | ##### | NA 175 | Insecticide | Microbial |
| N. sertifer | | | | | |
| Polyhedral occlusion bodies | NA 835 | ##### | 4028 | Insecticide | Microbial |
| (OB's) of the nuclear | | | | | |
| polyhedrosis virus of | | | | | |
| Helicoverpa zea (corn | | | | | |
| earworm) | | | | | |
| Polyhedral occlusion bodies | NA 1292 | ##### | NA 175 | Insecticide | Microbial |
| (OBs) of the nuclear | | | | | |
| polyhedrosis virus of | | | | | |
| Autographa californica (alfalfa | | | | | |
| looper) | | | | | |
| Polyhedral occlusion bodies | NA 830 | NA 153 | 4007 | Insecticide | Microbial |
| (OBs) of the nuclear | | | | | |
| polyhedrosis virus of | | | | | |
| Spodoptera exigua | | | | | |
| Polyhedral occlusion bodies of | NA 1293 | ##### | NA 175 | Insecticide | Microbial |
| the beet armyworm nuclear | | | | | |
| polyhedrosis virus | | | | | |
| Pseudomonas aureofaciens | NA 1296 | 6473 | NA 175 | Fungicide | Microbial |
| strain Tx-1 | | | | | |
| Pseudomonas cepacia type | NA 449 | 6419 | 3926 | Fungicide, | Microbial |
| Wisconsin | | | | Nematicide | |
| Pseudomonas chlororaphis | NA 902 | NA 172 | 5182 | Fungicide | Microbial |
| Pseudomonas chlororaphis | NA 1489 | 6478 | NA 220 | Fungicide | Microbial |
| strain 63-28 | | | | | |
| Pseudomonas fluorescens EG- | NA 858 | 6440 | 5045 | Fungicide | Microbial |
| 1053 | | | | | |
| Pseudomonas fluorescens | NA 867 | 6439 | 3949 | Fungicide | Microbial |
| 1629RS | | | | | |
| Pseudomonas fluorescens | NA 1020 | 6438 | 2842 | Fungicide | Microbial |
| A506 | | | | | |
| Pseudomonas fluorescens | NA 868 | 6420 | 3968 | Fungicide | Microbial |
| strain NCIB 12089 | | | | | |
| Pseudomonas syringae strain | NA 897 | NA 171 | 3950 | N/A | Microbial |
| AGS31 Psedomonas syringae | | | | | |
| strain PS31 | | | | | |
| Pseudomonas syringae strain | NA 836 | 6451 | 3967 | Fungicide | Microbial |
| ESC-11 | | | | | |
| Pseudomonas syringae, strain | 68602-93-7 | 6411 | 2824 | Fungicide | Microbial |
| 742 RS | | | | | |
| Pseudomonas syringae, strain | 68583-32-4 | 6441 | 3960 | Fungicide | Microbial |
| ESC-10 | | | | | |
| Puccinia canaliculata | NA 865 | ##### | 5121 | Herbicide | Microbial |
| (Schweinitz) Lagerheim | | | | | |
| (ATCC #40199) | | | | | |
| QST 713 strain of dried | NA 915 | 6479 | 5447 | Fungicide | Microbial |
| Bacillus subtilis | | | | | |
| Rabbit Calicivirus | NA 1441 | NA 214 | NA 213 | Rodent | Microbial |
| | | | | Repellent | |
| Rhizobium leguminosarum | NA 1556 | NA 226 | NA 226 | Insecticide | Microbial |
| biovar phaseoli | | | | | |
| Rhizobium leguminosarum | NA 1557 | NA 226 | NA 226 | Insecticide | Microbial |
| viciaeTJ 9 | | | | | |
| Rhizobium meliloti | NA 1558 | NA 227 | NA 226 | Insecticide | Microbial |
| Spinosad | 131929-60-7 | ##### | 3983 | Insecticide | Microbial |
| Spinosyn D | 131929-63-0 | ##### | NA 907 | Insecticide | Microbial |
| Spores and mycelium spores of | NA 1446 | NA 214 | NA 214 | Fungicide | Microbial |
| Phlebiopsis gigantea | | | | | |
| Spores of Bacillus popilliae | NA 1310 | 54502 | NA 176 | Fungicide | Microbial |
| Spores of Gliocladium | NA 1453 | NA 215 | NA 215 | Fungicide | Microbial |
| catenulatum | | | | | |
| Streptomyces griseoviridis | NA 875 | ##### | 3937 | Fungicide | Microbial |
| strain K61 | | | | | |
| Summerfruit tortrix moth | NA 1491 | NA 221 | NA 221 | Insecticide | Microbial |
| granulosis virus | | | | | |
| Tomato mosaic virus | NA 1387 | NA 196 | NA 195 | N/A | Microbial |
| Trichoderma harzianum | 67892-34-6 | ##### | NA 106 | Fungicide | Microbial |
| (ATCC 20476) | | | | | |
| Trichoderma harzianum rifai | 67892-31-3 | ##### | 3977 | Fungicide | Microbial |
| strain KRL-AG2 | | | | | |
| Trichoderma harzianum Rifai | 67892-31-3 | ##### | 4016 | Fungicide | Microbial |
| strain T-39 | | | | | |
| Trichoderma polysporum | 67892-31-3 | ##### | NA 176 | Fungicide | Microbial |
| (ATCC 20475) | | | | | |
| Trichoderma polysporum rifai | NA 1067 | NA 159 | 5101 | N/A | Microbial |
| Verticillium dahliae Kleb. | NA 1386 | NA 196 | NA 195 | Fungicide | Microbial |
| Verticillium lecanii | 67892-35-7 | 6421 | NA 96 | Insecticide | Microbial |
| Xanthomonas campestris pv. | NA 910 | NA 174 | 5317 | Herbicide | Microbial |
| Poannua | | | | | |
| Yeast | NA 652 | NA 119 | 3827 | N/A | Microbial |
| Allyl isothiocyanate | 57-06-7 | 4901 | 1010 | Insecticide | Oil - essential |
| Almond, bitter | 8013-76-1 | NA 546 | 3576 | N/A | Oil - essential |
| Aloe vera oil | NA 918 | NA 175 | 5454 | N/A | Oil - essential |
| Blend of oils: of lemongrass, of | NA 1499 | NA 221 | NA 221 | Insecticide | Oil - essential |
| citronella, of orange, of | | | | | |
| bergamot; geraniol, ionone | | | | | |
| alpha, methyl salicylate and | | | | | |
| allylisothioc | | | | | |
| Camphor | 76-22-2 | 15602 | 102 | Insecticide, | Oil - essential |
| | | | | Fungicide, | |
| | | | | Microbiocide | |
| Camphor oil | 8008-51-3 | 15601 | NA 142 | Insecticide, | Oil - essential |
| | | | | Fungicide, | |
| | | | | Microbiocide | |
| Canadian balsam | 8007-47-4 | 67209 | NA 580 | Insecticide, | Oil - essential |
| | | | | Fungicide | |
| Cedar leaf oil | 8007-20-3 | 40507 | NA 340 | Insecticide, | Oil - essential |
| | | | | Fungicide | |
| Cinnamon oil | NA 922 | NA 175 | 5577 | N/A | Oil - essential |
| Clove oil | 8000-34-8 | ##### | 5018 | N/A | Oil - essential |
| Essential oils | 8014-17-3 | NA 121 | 873 | Insecticide | Oil - essential |
| Eucalyptus oil | 8000-48-4 | 40503 | 281 | Insect | Oil - essential |
| | | | | Repellent, | |
| | | | | Insecticide | |
| Fir needle oil | 8021-28-1 | ##### | NA 110 | N/A | Oil - essential |
| Lavandin oil | 8022-15-9 | 40500 | 5017 | Insect | Oil - essential |
| | | | | Repellent, | |
| | | | | Insecticide, | |
| | | | | Dog and Cat | |
| | | | | Repellent | |
| Lemon peel oil | 8020-19-7 | 40518 | NA 346 | N/A | Oil - essential |
| Lime Oil | 8008-26-2 | NA 213 | NA 212 | Insecticide | Oil - essential |
| Limonene | 138-86-3 | 79701 | 979 | Insect | Oil - essential |
| | | | | Repellent, | |
| | | | | Insecticide, | |
| | | | | Dog and Cat | |
| | | | | Repellent | |
| Limonene | 138-86-3 | 79701 | 2531 | Insecticide | Oil - essential |
| Mixture citronella oil, citrus | NA 1493 | NA 221 | NA 221 | Insect | Oil - essential |
| oil, eucalyptus oil, pine oil | | | | Repellent | |
| Oil of ambergis | 8038-65-1 | ##### | NA 105 | N/A | Oil - essential |
| Oil of anise | 8007-70-3 | 4301 | 1051 | Insect | Oil - essential |
| | | | | Repellent, | |
| | | | | Insecticide, | |
| | | | | Dog and Cat | |
| | | | | Repellent | |
| Oil of bay | 8006-78-8 | 40520 | 2072 | N/A | Oil - essential |
| Oil of bergamot | 8007-75-8 | ##### | 1517 | Insect | Oil - essential |
| | | | | Repellent, | |
| | | | | Insecticide, | |
| | | | | Dog and Cat | |
| | | | | Repellent | |
| Oil of black pepper | NA 1521 | NA 223 | NA 223 | Insecticide | Oil - essential |
| Oil of cajeput | 8008-98-8 | 40504 | NA 339 | N/A | Oil - essential |
| Oil of camphor sassafrassy | 8006-80-2 | 15603 | 897 | N/A | Oil - essential |
| Oil of chamomile (Matricaria | 8002-66-2 | ##### | NA 104 | Insecticide | Oil - essential |
| chamomilla and/or Anthemidis | | | | | |
| nobilis) | | | | | |
| Oil of chenopodium | 8006-99-3 | NA 18 | 1524 | N/A | Oil - essential |
| Oil of citronella | 8000-29-1 | 21901 | 143 | Insect | Oil - essential |
| | | | | Repellent, Dog | |
| | | | | and Cat | |
| | | | | Repellent | |
| Oil of citrus | NA 13 | NA 19 | 1931 | Insect | Oil - essential |
| | | | | Repellent | |
| Oil of geranium | 8000-46-2 | ##### | 1887 | Fragrance | Oil - essential |
| Oil of jasmine | 8022-96-6 | 40501 | 5063 | Insecticide | Oil - essential |
| Oil of jojoba | 61789-91-1 | 67200 | 3833 | Insecticide, | Oil - essential |
| | | | | Insect | |
| | | | | Repellent, | |
| | | | | Fungicide, Dog | |
| | | | | and Cat | |
| | | | | Repellent | |
| Oil of lavender | 8000-28-0 | ##### | 1888 | Insecticide | Oil - essential |
| Oil of lemon | 8008-56-8 | NA 120 | 440 | N/A | Oil - essential |
| Oil of lemongrass | 5392-40-5 | 40510 | 1009 | Insecticide, | Oil - essential |
| | | | | Pheromone | |
| Oil of lemongrass | 2229107 | 40502 | 1136 | Insect | Oil - essential |
| | | | | Repellent, | |
| | | | | Insecticide | |
| Oil of mustard | 57-06-7 | 4901 | 1153 | Insect | Oil - essential |
| | | | | Repellent, | |
| | | | | Insecticide, | |
| | | | | Dog and Cat | |
| | | | | Repellent | |
| Oil of orange | 8008-57-9 | 40517 | 441 | Insect | Oil - essential |
| | | | | Repellent, | |
| | | | | Insecticide, | |
| | | | | Dog and Cat | |
| | | | | Repellent | |
| Oil of pennyroyal | 8007-44-1 | 40509 | 1325 | Insecticide | Oil - essential |
| Oil of peppermint | 8006-90-4 | NA 669 | 2058 | Insecticide | Oil - essential |
| Oil of rosemary | 8000-25-7 | ##### | 5450 | Insecticide | Oil - essential |
| Oil of rue | 8014-29-7 | 40519 | 2206 | N/A | Oil - essential |
| Oil of spearmint | 8008-79-5 | ##### | 1508 | N/A | Oil - essential |
| Oil of thyme | 8007-46-3 | ##### | NA 159 | N/A | Oil - essential |
| Oil of wintergreen | 119-36-8 | 76601 | 746 | Insecticide | Oil - essential |
| Oils, tea-tree | 68647-73-4 | 28853 | NA 211 | N/A | Oil - essential |
| Orange pith oil | NA 1461 | NA 217 | NA 216 | Insecticide | Oil - essential |
| Perfume, cherry fragrance oil | NA 751 | NA 136 | 2743 | Fragrance | Oil - essential |
| #493 | | | | | |
| Phytelene of marigold | 70892-20-5 | ##### | NA 976 | N/A | Oil - essential |
| Phytelene of marigold | 84776-23-8 | ##### | NA 977 | N/A | Oil - essential |
| Tansy, sage and lavender | NA 1452 | NA 215 | NA 214 | Insecticide | Oil - essential |
| extracts | | | | | |
| Vanillin | 121-33-5 | ##### | 5149 | N/A | Oil - essential |
| Canola oil | 120962-03-0 | 11332 | 5332 | Insecticide, | Oil - vegetable |
| | | | | Adjuvant | |
| Castor oil | 8001-79-4 | 31608 | 1013 | Rodenticide, | Oil - vegetable |
| | | | | Insecticide, | |
| | | | | Insect | |
| | | | | Repellent | |
| Coconut oil | 8001-31-8 | ##### | NA 124 | N/A | Oil - vegetable |
| Coconut oil monoethanolamine | 68140-00-1 | 69179 | 1361 | Adjuvant, | Oil - vegetable |
| | | | | Soap/Surfactant | |
| Corn oil | 8001-30-7 | NA 117 | 3626 | N/A | Oil - vegetable |
| Cottonseed oil | 8001-29-4 | 31602 | 1015 | Insecticide | Oil - vegetable |
| Emulsifiable methylated | NA 907 | NA 173 | 5224 | Insecticide, | Oil - vegetable |
| vegetable oil | | | | Adjuvant | |
| Fir needle oil, siberian | 8021-29-2 | NA 236 | 2570 | N/A | Oil - vegetable |
| Hydrogenated castor oil | 8001-78-3 | 31604 | 3830 | Bird Repellent | Oil - vegetable |
| Hydrogenated soybean oil | 8016-70-4 | NA 186 | 3670 | N/A | Oil - vegetable |
| Hydrogenated vegetable oils | NA 881 | NA 169 | 5146 | N/A | Oil - vegetable |
| Linseed oil with driers | NA 61 | NA 116 | 2645 | N/A | Oil - vegetable |
| Linseed oil, boiled | 68553-15-1 | NA 115 | 2644 | N/A | Oil - vegetable |
| Methyl soyate | 68919-53-9 | NA 146 | 3519 | Adjuvant | Oil - vegetable |
| Oil of linseed | 8001-26-1 | 31603 | 2248 | N/A | Oil - vegetable |
| Olive oil | 8001-25-0 | 31610 | 3710 | N/A | Oil - vegetable |
| Palm oil | NA 749 | NA 136 | 2731 | N/A | Oil - vegetable |
| Peanut oil | 2227315 | NA 118 | 3722 | N/A | Oil - vegetable |
| Plant oils (including rape seed | NA 1351 | NA 192 | NA 191 | Insecticide | Oil - vegetable |
| oil) | | | | | |
| Rapeseed oil | 8002-13-9 | NA 220 | NA 219 | Insecticide | Oil - vegetable |
| Safflower oil | 8001-23-8 | NA 124 | 1510 | N/A | Oil - vegetable |
| Sesame oil | 8008-74-0 | 72401 | 1221 | N/A | Oil - vegetable |
| Soybean oil | 8001-22-7 | 31605 | 2335 | Insecticide, | Oil - vegetable |
| | | | | Adjuvant | |
| Vegetable oil | 8008-89-7 | NA 111 | 1231 | Insecticide | Oil - vegetable |
| Vegetable oil modified | NA 552 | NA 100 | 2981 | N/A | Oil - vegetable |
| phenolic resin | | | | | |
| Vegetable wax | 8001-39-6 | 99501 | 1441 | N/A | Oil - vegetable |
| Weed oil | NA 558 | NA 101 | 1694 | N/A | Oil - vegetable |
| Wheat germ oil | NA 559 | NA 101 | 2988 | N/A | Oil - vegetable |
| (1R-exo)-Brevicomin | 20290-99-7 | ##### | NA 106 | Pheromone | Pheromone |
| (4E-7Z)-4,7-Tridecadien-1-yl- | NA 1428 | NA 199 | NA 198 | Pheromone | Pheromone |
| acetate | | | | | |
| (4Z-9Z)-7,9-Dodecadien-1-ol | NA 1412 | NA 198 | NA 197 | Pheromone | Pheromone |
| (E)-5-decen-1-ol | 56578-18-8 | 78038 | 5315 | Pheromone | Pheromone |
| (E)-10-Dodecenyl acetate | 35153-09-4 | NA 187 | NA 186 | Pheromone | Pheromone |
| (E)-11-Tetradecenyl acetate | 33189-72-9 | ##### | 5022 | Pheromone | Pheromone |
| (E)-2,6-dimethyl-2,6- | 141-27-5 | NA 21 | 3307 | Pheromone | Pheromone |
| octadienal | | | | | |
| (E)-4-tridecen-1-yl-acetate | 72269-48-8 | ##### | 2199 | Pheromone | Pheromone |
| (E)-5-Decenol | 56578-18-8 | 78038 | 3974 | Pheromone | Pheromone |
| (E)-5-Decenyl acetate | 38421-90-8 | ##### | 3975 | Pheromone | Pheromone |
| (E)-6-dodecenyl acetate | 29868-16-4 | ##### | NA 958 | Pheromone | Pheromone |
| (E)-9-decen-1-ol | 56578-18-8 | 78038 | 5071 | Pheromone | Pheromone |
| (E)-9-dodecen-1-ol, acetate | 35148-19-7 | ##### | 2141 | Pheromone | Pheromone |
| (E)-9-Tricosene | 35857-62-6 | NA 200 | NA 199 | Pheromone | Pheromone |
| (E)7-(Z)9-Dodecadienyl | NA 1413 | NA 198 | NA 197 | Pheromone | Pheromone |
| acetate | | | | | |
| (E,Z)-3,13-octadecadien-1-ol | 53120-26-6 | ##### | NA 955 | Pheromone | Pheromone |
| acetate | | | | | |
| (E/Z)-8-Dodecenyl acetate | NA 1415 | NA 198 | NA 197 | Pheromone | Pheromone |
| (R,Z)-5-(1-decenyl) dihydro-2- | 64726-91-6 | ##### | 2096 | Pheromone | Pheromone |
| (3H)-furanone | | | | | |
| (Z)-(3,3- | 26532-24-1 | ##### | 5081 | Pheromone | Pheromone |
| dimethylcyclohexylidene)acetaldehyde | | | | | |
| (Z)-11-hexadecen-1-ol | 56683-54-6 | ##### | NA 971 | Pheromone | Pheromone |
| (Z)-11-Hexadecen-1-ol | 56683-54-6 | NA 193 | NA 192 | Pheromone | Pheromone |
| (Z)-11-hexadecenal | 53939-28-9 | ##### | 2126 | Pheromone | Pheromone |
| (Z)-11-hexadecenyl acetate | 60037-58-3 | ##### | NA 111 | Pheromone | Pheromone |
| (Z)-11-Tetradecen-1-yl-acetate | 20711-10-8 | NA 199 | NA 199 | Pheromone | Pheromone |
| (Z)-11-Tetradecenyl acetate | 20711-10-8 | ##### | 5023 | Pheromone | Pheromone |
| (Z)-13-Octadecanol | NA 1423 | NA 199 | NA 198 | Pheromone | Pheromone |
| (Z)-13-Octadecenal | 58594-45-9 | ##### | NA 111 | Pheromone | Pheromone |
| (Z)-13-Octadecenyl acetate | 34010-21-4 | ##### | 5316 | Pheromone | Pheromone |
| (Z)-2-(3,3-dimethyl cyclo | 26532-23-0 | ##### | 5079 | Pheromone | Pheromone |
| hexylidene)ethanol | | | | | |
| (Z)-3-Methyl-6-isopropenyl--9- | NA 1420 | NA 198 | NA 198 | Pheromone | Pheromone |
| decen-1-yl acetate | | | | | |
| (Z)-3-Methyl-6-isopropenyl- | NA 1419 | NA 198 | NA 198 | Pheromone | Pheromone |
| 3,4-decadien-1-yl | | | | | |
| (Z)-4-tridecen-l-yl-acetate | 65954-19-0 | ##### | 2200 | Pheromone | Pheromone |
| (Z)-5-Decenyl acetate | 67446-07-5 | NA 198 | NA 197 | Pheromone | Pheromone |
| (Z)-5-Dodecen-1-yl acetate | 16676-96-3 | NA 187 | NA 186 | Pheromone | Pheromone |
| (Z)-6-dodecenyl acetate | 16974-12-2 | ##### | NA 957 | Pheromone | Pheromone |
| (Z)-6-Heneicosane-11-one | 54844-65-4 | ##### | NA 110 | Pheromone | Pheromone |
| (Z)-7-hexadecenal | 56797-40-1 | ##### | NA 972 | Pheromone | Pheromone |
| (Z)-7-Tetradecanol | NA 1427 | NA 199 | NA 198 | Pheromone | Pheromone |
| (Z)-7-Tetradecenal | 65128-96-3 | NA 187 | NA 186 | Pheromone | Pheromone |
| (Z)-9-dodecenyl acetate | 16974-11-1 | ##### | 2142 | Pheromone | Pheromone |
| (Z)-9-hexadecenal | 56219-04-6 | ##### | NA 973 | Pheromone | Pheromone |
| (Z)-9-tetradecen-1-ol | 35153-15-2 | ##### | 5618 | Pheromone | Pheromone |
| (Z)-9-tetradecenal | 53939-27-8 | ##### | 2209 | Pheromone | Pheromone |
| (Z)-9-Tetradecenyl acetate | 16725-53-4 | NA 187 | NA 186 | Pheromone | Pheromone |
| (Z)-9-Tricosene | 27519-02-4 | ##### | NA 883 | Pheromone | Pheromone |
| (Z,E) 7,11-hexadecaclien-1-ol | NA 1144 | NA 168 | 91997 | Pheromone | Pheromone |
| acetate, other related | | | | | |
| (Z,E)-7,11-hexadecadien-1-yl | 51607-94-4 | ##### | 1997 | Pheromone | Pheromone |
| acetate | | | | | |
| (Z,E)-7,11-hexadecadien-1-yl | 53042-79-8 | ##### | NA 933 | Pheromone | Pheromone |
| acetate | | | | | |
| (Z,E)-8,10-dodecadienyl | NA 1561 | NA 227 | NA 227 | Pheromone | Pheromone |
| acetate | | | | | |
| (Z,E)-9,11-Tetradecadien-1-yl | 50767-79-8 | NA 199 | NA 198 | Pheromone | Pheromone |
| acetate | | | | | |
| (Z,E)-9,12-tetradecadienyl | 31654-77-0 | ##### | 5617 | Pheromone | Pheromone |
| acetate | | | | | |
| (Z,Z) 7,11-hexadecadien-1-ol | NA 1145 | NA 168 | 91998 | Pheromone | Pheromone |
| acetate, other related | | | | | |
| (Z,Z) Octadienyl acetate | NA 1422 | NA 199 | NA 198 | Pheromone | Pheromone |
| (Z,Z)-11,13-hexadecadienal | 71317-73-2 | 711 | 5314 | Pheromone | Pheromone |
| (Z,Z)-3,13-octadecadien-1-ol | 53120-27-7 | ##### | NA 954 | Pheromone | Pheromone |
| acetate | | | | | |
| (Z,Z)-7,11-hexadecadien-1-yl | 52207-99-5 | ##### | 1998 | Pheromone | Pheromone |
| acetate | | | | | |
| (Z,Z)-7,11-Hexadecadienyl | 52207-99-5 | NA 198 | NA 197 | Pheromone | Pheromone |
| acetate | | | | | |
| 1,7-Dioxaspiro-5,5-undecane | 180-84-7 | NA 186 | NA 186 | Pheromone | Pheromone |
| 1-Methylbutyl decanoate | 55195-26-1 | ##### | NA 100 | Pheromone | Pheromone |
| 1-Octen-3-ol | 3391-86-4 | 69037 | 4029 | Pheromone, | Pheromone |
| | | | | Fragrance, | |
| | | | | Insect | |
| | | | | Repellent, Dog | |
| | | | | and Cat | |
| | | | | Repellent | |
| 11-(E)-Tetradecen-1-ol | 35153-18-5 | ##### | 5108 | Pheromone | Pheromone |
| 11-(Z)-Tetradecen-1-ol | 34010-15-6 | ##### | NA 109 | Pheromone | Pheromone |
| 2-cyclopenten-1-one, 2- | 80-71-7 | 4049 | NA 68 | Pheromone, | Pheromone |
| hydroxy-3-methyl- | | | | Fragrance | |
| 2-methyl-7(R),8(S)-epoxy | 29804-22-6 | ##### | 2165 | Pheromone | Pheromone |
| octadecane | | | | | |
| 3(S)-Methyl-6-isopropenyl-9- | NA 1535 | NA 224 | NA 224 | Pheromone | Pheromone |
| dodecadien-1yl acetate | | | | | |
| 3,7,11-trimethyl-2,6,10- | 4601-84-0 | NA 983 | 2269 | Pheromone | Pheromone |
| dodecatriene-1-ol | | | | | |
| 3-Methyl-2-cyclohexen-1-one | 1193-18-6 | ##### | NA 124 | Insect | Pheromone |
| | | | | Repellent, | |
| | | | | Pheromone | |
| 4-(p-acetoxyphenyl)-2- | 609379 | ##### | NA 106 | Pheromone | Pheromone |
| butanone | | | | | |
| 4-Methyl-3-heptanol | 14979-39-6 | ##### | NA 962 | Pheromone | Pheromone |
| 7,11-hexadecadien-1-ol, acetate | 50933-33-0 | ##### | NA 934 | Pheromone | Pheromone |
| 7,8-Epoxi-2-methyl-octadecane | NA 1417 | NA 198 | NA 197 | Pheromone | Pheromone |
| 8-dodecene-1-ol, other related | NA 1150 | NA 168 | 92253 | Pheromone | Pheromone |
| 9-tetradecen-1-ol, formate, (Z)- | 56218-79-2 | ##### | NA 967 | Pheromone | Pheromone |
| Acetaldehyde,(3,3- | 26532-25-2 | ##### | 5080 | Pheromone | Pheromone |
| dimethylcyclohexylidene)-,(E) | | | | | |
| beta-Farnesene | 18794-84-8 | ##### | NA 110 | Pheromone | Pheromone |
| Cis-11-tetradecenyl acetate | 20711-10-8 | ##### | 5038 | Pheromone | Pheromone |
| Distilled cubeb oil | 8007-87-2 | ##### | NA 960 | Pheromone | Pheromone |
| Dodecadienal | NA 1547 | NA 226 | NA 225 | Pheromone | Pheromone |
| E,E-8,10-dodecadien-1-ol | 33956-49-9 | ##### | 1851 | Pheromone | Pheromone |
| E-3,3-dimethyl-delta,alpha- | NA 951 | NA 315 | 2193 | Pheromone | Pheromone |
| cyclohexane ethanal | | | | | |
| E-8-dodecene | NA 1532 | NA 224 | NA 224 | Pheromone | Pheromone |
| E-8-dodecenyl acetate | 38363-29-0 | ##### | 2252 | Pheromone | Pheromone |
| Farnesol | 4602-84-0 | ##### | 2564 | Pheromone | Pheromone |
| Frontalin | 28401-39-0 | ##### | NA 985 | Pheromone | Pheromone |
| Frontalin + alpha-pinene | 12701-72-3 | ##### | NA 986 | Pheromone | Pheromone |
| mixture | | | | | |
| German cockroach pheromone | NA 871 | 29028 | 5132 | Pheromone | Pheromone |
| Isoamyl acetate | 123-92-2 | NA 167 | 1422 | Fragrance | Pheromone |
| Isomate LBAM | NA 1438 | NA 213 | NA 212 | Pheromone | Pheromone |
| Isomate OFM Rosso | NA 1426 | NA 213 | NA 212 | Pheromone | Pheromone |
| Lauryl alcohol | 112-53-8 | 1509 | 2343 | Pheromone, | Pheromone, |
| | | | | Adjuvant | Alcohol/Ether |
| Luretape (Grandlure) | 11104-05-5 | ##### | NA 923 | Pheromone | Pheromone |
| Methyl-trans-6-nonenoate | NA 1421 | NA 199 | NA 198 | Pheromone | Pheromone |
| Multilure | 60018-97-5 | ##### | NA 963 | Pheromone | Pheromone |
| Multistriatin | 59014-03-8 | ##### | NA 961 | Pheromone | Pheromone |
| Muscalure | 27519-02-4 | ##### | 1858 | Pheromone | Pheromone |
| Myrcene | 123-35-3 | ##### | 2189 | Pheromone | Pheromone |
| Nerolidol | 7212-44-4 | ##### | 2268 | Pheromone | Pheromone |
| Nerolidol | 7212-44-4 | ##### | 2701 | Pheromone | Pheromone |
| Pentyl valerate | 2173-56-0 | ##### | 1809 | Pheromone | Pheromone |
| Periplanone B | 61228-92-0 | ##### | 2766 | Pheromone | Pheromone |
| Pherodim | NA 1424 | NA 199 | NA 198 | Pheromone | Pheromone |
| Pronumone | NA 1425 | NA 199 | NA 198 | Pheromone | Pheromone |
| Serricornin | NA 1343 | NA 191 | NA 190 | Pheromone | Pheromone |
| Tetradecanal | 124-25-4 | ##### | NA 975 | Pheromone | Pheromone |
| Trans-11-tetradecenyl acetate | 33189-72-9 | ##### | 5039 | Pheromone | Pheromone |
| trans-6-Nonen-1-ol | NA 1404 | NA 197 | NA 197 | Pheromone | Pheromone |
| Trimedlure | 12002-53-8 | ##### | NA 199 | Pheromone | Pheromone |
| Z-2-isopropenyl-1-methyl | 26532-22-9 | ##### | 2190 | Pheromone | Pheromone |
| cyclobutane ethanol | | | | | |
| Z-3,3-dimethyl-delta,alpha- | NA 952 | NA 316 | 2192 | Pheromone | Pheromone |
| cyclohexane ethanal | | | | | |
| Z-3,3-dimethyl-delta,beta- | NA 706 | NA 129 | 2191 | Pheromone | Pheromone |
| cyclohexane ethanol | | | | | |
| Z-8-dodecene | NA 1531 | NA 224 | NA 224 | Pheromone | Pheromone |
| Z-8-dodecenol | 40642-40-8 | ##### | 2253 | Pheromone | Pheromone |
| Z-8-dodecenyl acetate | 28079-04-1 | ##### | 2251 | Pheromone | Pheromone |
| 7-(diethyl amino)-4-methyl | 91-44-1 | NA 341 | 3146 | Rodenticide | Coumarin |
| coumarin | | | | | |
| Brodifacoum | 56073-10-0 | ##### | 2049 | Rodenticide | Coumarin |
| Bromadiolone | 28772-56-7 | ##### | 2135 | Rodenticide | Coumarin |
| Coumachlor | 81-82-3 | ##### | NA 126 | Rodenticide | Coumarin |
| Coumafuryl | 117-52-2 | 86001 | 298 | Rodenticide | Coumarin |
| Coumafuryl, sodium salt | 34490-93-2 | 86004 | 299 | Rodenticide | Coumarin |
| Coumarin | 91-64-5 | ##### | NA 102 | Rodenticide | Coumarin |
| Coumatetralyl | 5836-29-3 | ##### | NA 153 | Rodenticide | Coumarin |
| Difenacoum | 56073-07-5 | ##### | NA 970 | Rodenticide | Coumarin |
| Flocoumafen | 90035-08-8 | NA 182 | NA 181 | Rodenticide | Coumarin |
| Potassium warfarin | 2610-86-8 | 86005 | NA 836 | Rodenticide | Coumarin |
| Pyranocoumarin | NA 1431 | NA 199 | NA 199 | Rodenticide | Coumarin |
| Warfarin | 81-81-2 | 86002 | 621 | Rodenticide | Coumarin |
| Warfarin, sodium salt | 129-06-6 | 86003 | 1184 | Rodenticide | Coumarin |
| 2-isovaleryl 1-1,3-indandione | 83-28-3 | 67702 | 1435 | Rodenticide | 1,3-Indandione |
| 2-isovaleryl 1-1,3-indandione, | 23710-76-1 | 67706 | 1736 | Rodenticide | 1,3-Indandione |
| calcium salt | | | | | |
| 2-isovaleryl-1,3-indandione, | 53404-57-2 | 67709 | NA 596 | Rodenticide | 1,3-Indandione |
| sodium salt | | | | | |
| Chlorophacinone | 3691-35-8 | 67707 | 1625 | Rodenticide | 1,3-Indandione |
| Diphacinone | 82-66-6 | 67701 | 225 | Rodenticide | 1,3-Indandione |
| Diphacinone, sodium salt | 42721-99-3 | 67705 | 1636 | Rodenticide | 1,3-Indandione |
| Pindone | 83-26-1 | 67703 | 489 | Rodenticide | 1,3-Indandione |
| Pindone, calcium salt | 53404-72-1 | 67708 | NA 595 | Rodenticide | 1,3-Indandione |
| Pindone, sodium salt | 6120-20-3 | 67704 | 1305 | Rodenticide, | 1,3-Indandione |
| | | | | Bird Repellent | |
| 4-Hydroxymethyl | NA 1173 | NA 177 | NA 165 | Breakdown | 2,6-Dinitroaniline |
| pendimethalin | | | | product | |
| Benfluralin | 1861-40-1 | 84301 | 53 | Herbicide | 2,6-Dinitroaniline |
| Butralin | 33629-47-9 | ##### | 1756 | Herbicide | 2,6-Dinitroaniline |
| Dinitramine | 29091-05-2 | ##### | 1683 | Herbicide | 2,6-Dinitroaniline |
| Dipropalin | 5332 | ##### | NA 145 | Herbicide | 2,6-Dinitroaniline |
| Ethalfluralin | 55283-68-6 | ##### | 2166 | Herbicide | 2,6-Dinitroaniline |
| Fluazinam | 79622-59-6 | ##### | 3898 | Fungicide | 2,6-Dinitroaniline |
| Fluchloralin | 33245-39-5 | ##### | 1848 | Herbicide | 2,6-Dinitroaniline |
| Flumetralin | 62924-70-3 | ##### | 5085 | Plant Growth | 2,6-Dinitroaniline |
| | | | | Regulator | |
| Isopropalin | 33820-53-0 | ##### | 1681 | Herbicide | 2,6-Dinitroaniline |
| N,N-Bis(2-chloroethyl)-4- | 26389-78-6 | ##### | NA 135 | N/A | 2,6-Dinitroaniline |
| methyl-2,6-dinitroaniline | | | | | |
| Nitralin | 4726-14-1 | 37601 | 490 | Herbicide | 2,6-Dinitroaniline |
| Oryzalin | 19044-88-3 | ##### | 1868 | Herbicide | 2,6-Dinitroaniline |
| Pendimethalin | 40487-42-1 | ##### | 1929 | Herbicide | 2,6-Dinitroaniline |
| Prodiamine | 29091-21-2 | ##### | 2236 | Herbicide | 2,6-Dinitroaniline |
| Prodiamine, other related | NA 1149 | NA 168 | 92236 | Herbicide | 2,6-Dinitroaniline |
| Profluralin | 26399-36-0 | ##### | 1897 | Herbicide | 2,6-Dinitroaniline |
| Profluralin, other related | NA 1141 | NA 167 | 91897 | Herbicide | 2,6-Dinitroaniline |
| Trifluralin | 1582-09-8 | 36101 | 597 | Herbicide | 2,6-Dinitroaniline |
| 1-(3- | 51971-67-6 | ##### | NA 110 | N/A | Carboxamide |
| chlorophthalimido)cyclohexane | | | | | |
| carboxamide | | | | | |
| Allidochlor | 93-71-0 | 19301 | 114 | Herbicide | Amide |
| Allidochlor | 202-270-7 | NA 210 | NA 210 | Herbicide | Amide |
| Ammonium benzadox | 5251-79-6 | 98201 | NA 858 | Herbicide | Amide |
| Beflubutamid | 113614-08-7 | NA 230 | NA 230 | Herbicide | Amide |
| Benzadox | 5251-93-4 | ##### | NA 132 | Herbicide | Amide |
| Benzipram | 35256-86-1 | ##### | NA 133 | Herbicide | Amide |
| Bromobutide | 74712-19-9 | NA 203 | NA 203 | Herbicide | Amide |
| Carboxin | 5234-68-4 | 90201 | 1755 | Fungicide | Carboxamide |
| Chlorthiamid | 1918-13-4 | ##### | NA 139 | Herbicide | Amide |
| Cisanilide | 34484-77-0 | ##### | NA 144 | Herbicide | Carboxamide |
| Cyclafuramide | 34849-42-8 | ##### | NA 132 | Fungicide | Carboxamide |
| Cyprazole | 42089-03-2 | ##### | NA 132 | Herbicide | Amide |
| Dimethenamid | 87674-68-8 | ##### | 5112 | Herbicide | Amide |
| Diphenamid | 957-51-7 | 36601 | 226 | Herbicide | Amide |
| Epronaz | 59026-08-3 | NA 231 | NA 231 | Herbicide | Amide |
| Etnipromid | 76120-02-0 | NA 231 | NA 231 | Herbicide | Amide |
| Fenfuram | 24691-80-3 | NA 188 | NA 187 | Fungicide | Carboxamide |
| Fentrazamide | 158237-07-1 | NA 232 | NA 231 | Herbicide | Amide |
| Flupoxam | 119126-15-7 | NA 188 | NA 188 | Herbicide | Amide |
| Furmecyclox (Xyligen B) | 60568-05-0 | ##### | NA 18 | Fungicide | Carboxamide |
| Halosafen | 77227-69-1 | NA 229 | NA 229 | Herbicide | Amide, Nitrophenyl |
| | | | | | ether |
| Isocarbamide | 30979-48-7 | NA 183 | NA 182 | Herbicide | Amide |
| Isoxaben | 82558-50-7 | ##### | 2289 | Herbicide | Amide |
| Methfuroxam | 28730-17-8 | ##### | NA 949 | Fungicide | Carboxamide |
| Metsulfovax | 21452-18-6 | NA 189 | NA 188 | Fungicide | Carboxamide |
| Napropamide | 15299-99-7 | ##### | 1728 | Herbicide | Amide |
| Naptalam | 132-66-1 | 30702 | 2998 | Herbicide | Amide |
| Naptalam, sodium salt | 132-67-2 | 30703 | 437 | Herbicide | Amide |
| Nitrofurazone (not subject to | 59-87-0 | ##### | NA 128 | N/A | Carboxamide |
| FIFRA: 8709-e pm 24) | | | | | |
| Oxycarboxin | 5259-88-1 | 90202 | 1434 | Fungicide | Carboxamide |
| p-Benzoquinone | 61566-21-0 | ##### | NA 133 | N/A | Carboxamide |
| semicarbazone | | | | | |
| Pethoxamid | 106700-29-2 | NA 233 | NA 233 | Herbicide | Amide |
| Propyzamide | 23950-58-5 | ##### | 694 | Herbicide | Amide |
| Propyzamide metabolite | NA 1048 | NA 155 | 4093 | Breakdown | Amide |
| | | | | product | |
| Quinonamid | 27541-88-4 | NA 210 | NA 209 | Herbicide | Amide |
| 2-Hydroxy alachlor | NA 943 | NA 183 | 2602 | Breakdown | Chloroacetanilide |
| | | | | product | |
| 3,4,5-tribromo salicylanilide, | NA 1122 | NA 165 | 90833 | Microbiocide | Anilide |
| other related | | | | | |
| 3,5-dibromosalicylanilide | 2577-72-2 | 77405 | 1256 | Microbiocide | Anilide |
| 5,4-dibromosalicylanilide | NA 1024 | NA 122 | 1071 | Microbiocide | Anilide |
| Aatram (propachlor with | 8070-76-6 | 80816 | NA 781 | Herbicide | Triazine, |
| atrazine) (019101 + 080803) | | | | | Chloroacetanilide |
| Acetochlor | 34256-82-1 | ##### | 2349 | Herbicide | Chloroacetanilide |
| Alachlor | 15972-60-8 | 90501 | 678 | Herbicide | Chloroacetanilide |
| Benodanil | 15310-01-7 | ##### | NA 154 | Fungicide | Anilide |
| Butachlor | 23184-66-9 | ##### | 4056 | Herbicide | Chloroacetanilide |
| Butenachlor | 87310-56-3 | NA 206 | NA 206 | Herbicide | Chloroacetanilide |
| Clomeprop | 84496-56-0 | NA 204 | NA 203 | Herbicide | Anilide |
| Cyprofuram | 69581-33-5 | NA 186 | NA 185 | Fungicide | Anilide |
| Cypromid | 2759-71-9 | 26101 | 178 | Herbicide | Anilide |
| Delachlor | 24353-58-0 | ##### | NA 132 | Herbicide | Chloroacetanilide |
| Diethatyl-ethyl | 38727-55-8 | ##### | 1995 | Herbicide | Chloroacetanilide |
| Diflufenican | 83164-33-4 | NA 184 | NA 183 | Herbicide | Anilide |
| Dimethachlor | 50563-36-5 | NA 184 | NA 183 | Herbicide | Chloroacetanilide |
| Etobenzanid | 79540-50-4 | NA 229 | NA 228 | Herbicide | Anilide |
| Fenasulam | 78357-48-9 | NA 231 | NA 231 | Herbicide | Anilide |
| Flufenacet | 142459-58-3 | ##### | 5293 | Herbicide | Anilide |
| Flufenican | NA 1568 | NA 234 | NA 234 | Herbicide | Anilide |
| Flutolanil | 66332-96-5 | ##### | 2305 | Fungicide | Anilide |
| Mefenacet | 73250-68-7 | NA 189 | NA 188 | Herbicide | Anilide |
| Mefluidide | 53780-34-0 | ##### | 5082 | Herbicide, | Anilide |
| | | | | Plant Growth | |
| | | | | Regulator | |
| Mefluidide, diethanolamine salt | 53780-36-2 | ##### | 1955 | Herbicide | Anilide |
| Mefluidide, potassium salt | 83601-83-6 | ##### | 5083 | Herbicide | Anilide |
| Mepronil | 55814-41-0 | NA 189 | NA 188 | Fungicide | Anilide |
| Metazachlor | 67129-08-2 | NA 184 | NA 183 | Herbicide | Chloroacetanilide |
| Metolachlor | 51218-45-2 | ##### | 1996 | Herbicide | Chloroacetanilide |
| Metolachlor, (S) | 87392-12-9 | ##### | 5133 | Herbicide | Chloroacetanilide |
| Monalide | 7287-36-7 | ##### | NA 134 | Herbicide | Anilide |
| Naproanilide | 52570-16-8 | NA 233 | NA 232 | Herbicide | Anilide |
| Ofurace | 58810-48-3 | NA 189 | NA 188 | Fungicide | Anilide |
| Oxadixyl | 77732-09-3 | ##### | 3539 | Fungicide | Anilide |
| Perfluidone | 37924-13-3 | ##### | 1895 | Herbicide | Anilide |
| Perfluidone, diethanolamine | NA 991 | NA 671 | 1896 | Herbicide | Anilide |
| salt | | | | | |
| Pretilachlor | 51218-49-6 | NA 184 | NA 184 | Herbicide | Chloroacetanilide |
| Propachlor | 1918-16-7 | 19101 | 511 | Herbicide | Chloroacetanilide |
| Propanil | 709-98-8 | 28201 | 503 | Herbicide | Anilide |
| Propisochlor | 86763-47-5 | NA 233 | NA 233 | Herbicide | Chloroacetanilide |
| Prynachlor | 21267-72-1 | ##### | NA 138 | Herbicide, | Chloroacetanilide |
| | | | | Plant Growth | |
| | | | | Regulator | |
| Pyracarbolid | 24691-76-7 | NA 210 | NA 209 | Fungicide | Anilide |
| Salicylanilide | 87-17-2 | 77407 | 450 | Fungicide, | Anilide |
| | | | | Microbiocide | |
| Sodium salicylanilide | 251930 | ##### | NA 145 | Fungicide | Anilide |
| Thenylchlor | 96491-05-3 | NA 229 | NA 229 | Herbicide | Chloroacetanilide |
| Triclocarban | 101-20-2 | 27901 | 844 | N/A | Anilide |
| Benzoylprop | 22212-56-2 | NA 182 | NA 181 | Herbicide | Arylalanine |
| Benzoylprop ethyl | 22212-55-1 | ##### | NA 147 | Herbicide | Arylalanine |
| Benzoylprop-ethyl | 33878-50-1 | NA 206 | NA 205 | Herbicide | Arylalanine |
| Flamprop | 58667-63-3 | NA 188 | NA 187 | Herbicide | Arylalanine |
| Flamprop-isopropyl | 52756-22-6 | ##### | NA 154 | Herbicide | Arylalanine |
| Flamprop-M-isopropyl | 57973-67-8 | ##### | NA 154 | Herbicide | Arylalanine |
| Flamprop-M-methyl | 57973-66-7 | ##### | NA 104 | Herbicide | Arylalanine |
| Flamprop-methyl | 52756-25-9 | ##### | NA 154 | Herbicide | Arylalanine |
| Chlorazifop | 60074-25-1 | NA 231 | NA 230 | Herbicide | Aryloxyphenoxy |
| | | | | | propionic acid |
| Clodinafop | 114420-56-3 | NA 182 | NA 182 | Plant Growth | Aryloxyphenoxy |
| | | | | Regulator | propionic acid |
| Clodinafop-propargyl | 105512-06-9 | ##### | NA 163 | Plant Growth | Aryloxyphenoxy |
| | | | | Regulator | propionic acid |
| Clofop | 26129-32-8 | NA 207 | NA 206 | Herbicide | Aryloxyphenoxy |
| | | | | | propionic acid |
| Cyhalofop | 122008-78-0 | NA 204 | NA 203 | Herbicide | Aryloxyphenoxy |
| | | | | | propionic acid |
| Cyhalofop butyl | 122008-85-9 | NA 215 | 5748 | Herbicide | Aryloxyphenoxy |
| | | | | | propionic acid |
| Diclofop | 40843-25-2 | ##### | NA 912 | Herbicide | Aryloxyphenoxy |
| | | | | | propionic acid |
| Diclofop-methyl | 51338-27-3 | ##### | 2034 | Herbicide | Aryloxyphenoxy |
| | | | | | propionic acid |
| Fenoxaprop-ethyl | 82110-72-3 | NA 201 | NA 200 | Herbicide | Aryloxyphenoxy |
| (stereochemistry unspecified) | | | | | propionic acid |
| Fenoxaprop-P (+) | 71283-80-2 | ##### | 5123 | Herbicide | Aryloxyphenoxy |
| | | | | | propionic acid |
| Fenoxaprop-P (+/−) | 66441-23-4 | ##### | 2311 | Herbicide | Aryloxyphenoxy |
| | | | | | propionic acid |
| Fenoxaprop-P-ethyl | 113158-40-0 | NA 220 | NA 219 | Herbicide | Aryloxyphenoxy |
| | | | | | propionic acid |
| Fenthiaprop (racemic) | 95721-12-3 | NA 207 | NA 207 | Herbicide | Aryloxyphenoxy |
| | | | | | propionic acid |
| Fenthiaprop (unstated | 73519-50-3 | NA 218 | NA 217 | Herbicide | Aryloxyphenoxy |
| stereochemistry) | | | | | propionic acid |
| Fenthiaprop-ethyl (racemic) | 93921-16-5 | NA 218 | NA 217 | Herbicide | Aryloxyphenoxy |
| | | | | | propionic acid |
| Fenthiaprop-ethyl (unstated | 66441-11-0 | NA 218 | NA 217 | Herbicide | Aryloxyphenoxy |
| stereochemistry) | | | | | propionic acid |
| Fluazifop | 69335-91-7 | NA 194 | NA 193 | Herbicide | Aryloxyphenoxy |
| | | | | | propionic acid |
| Fluazifop-butyl | 69806-50-4 | ##### | 2186 | Herbicide | Aryloxyphenoxy |
| | | | | | propionic acid |
| Fluazifop-P-butyl | 79241-46-6 | ##### | NA 995 | Herbicide | Aryloxyphenoxy |
| | | | | | propionic acid |
| Fluazifop-P-butyl | 83066-88-0 | NA 228 | NA 228 | Herbicide | Aryloxyphenoxy |
| | | | | | propionic acid |
| Haloxyfop (unstated | 69806-34-4 | ##### | NA 100 | Herbicide | Aryloxyphenoxy |
| stereochemistry) | | | | | propionic acid |
| Haloxyfop-etotyl (unstated | 87237-48-7 | NA 200 | NA 199 | Herbicide | Aryloxyphenoxy |
| stereochemistry) | | | | | propionic acid, |
| | | | | | Glycol Ether |
| Haloxyfop-methyl (unstated | 690806-40-2 | ##### | NA 19 | Herbicide | Aryloxyphenoxy |
| stereochemistry) | | | | | propionic acid |
| Haloxyfop-R | 72619-32-0 | NA 191 | NA 190 | Herbicide | Aryloxyphenoxy |
| | | | | | propionic acid |
| Propaquizafop | 111479-05-1 | NA 185 | NA 184 | Herbicide | Aryloxyphenoxy |
| | | | | | propionic acid |
| Quizalofop | 76578-12-6 | NA 190 | NA 189 | Herbicide | Aryloxyphenoxy |
| | | | | | propionic acid |
| Quizalofop-ethyl | 76578-14-8 | ##### | 2226 | Herbicide | Aryloxyphenoxy |
| | | | | | propionic acid |
| Quizalofop-P | 94051-08-8 | NA 185 | NA 184 | Herbicide | Aryloxyphenoxy |
| | | | | | propionic acid |
| Quizalofop-P-ethyl | 100646-51-3 | NA 212 | NA 211 | Herbicide | Aryloxyphenoxy |
| | | | | | propionic acid |
| Quizalofop-p-tefuryl | 119738-06-6 | NA 202 | NA 202 | Herbicide | Aryloxyphenoxy |
| | | | | | propionic acid |
| Trifop | 58594-744 | NA 234 | NA 233 | Herbicide | Aryloxyphenoxy |
| | | | | | propionic acid |
| 2,5-Dichlorobenzoic acid | 50-79-3 | NA 224 | NA 224 | Fungicide | Benzoic acid |
| 2,5-Dichlorobenzoic acid | 2905-69-3 | NA 224 | NA 224 | Fungicide | Benzoic acid |
| methyl ester | | | | | |
| 3,5-dichlorobenzoic acid | 51-36-5 | NA 156 | 4066 | N/A | Benzoic acid |
| 4′-hydroxy-m-phenoxy benzoic | 35065-12-4 | NA 176 | 2609 | Fungicide, | Benzoic acid |
| acid | | | | Microbiocide | |
| 4-hydroxy benzoic acid | 99-96-7 | NA 200 | NA 199 | N/A | Benzoic acid |
| 5-hydroxy-dicamba | NA 840 | NA 156 | 4065 | Herbicide, | Benzoic acid |
| | | | | Breakdown | |
| | | | | product | |
| Butyl paraben | 94-26-8 | 61205 | NA 496 | Fungicide, | Benzoic acid |
| | | | | Microbiocide | |
| Chloramben | 133-90-4 | 29901 | 2184 | Herbicide | Benzoic acid |
| Chloramben, ammonium salt | 1076-46-6 | 29902 | 831 | Herbicide | Benzoic acid |
| Chloramben, ammonium salt, | NA 1121 | NA 165 | 90831 | Herbicide | Benzoic acid |
| other related | | | | | |
| Dicamba | 1918-00-9 | 29801 | 200 | Herbicide | Benzoic acid |
| Dicamba with 2,4-D | 8068-77-7 | 29807 | NA 219 | Herbicide | Benzoic acid |
| Dicamba with 2,4-D & Silvex | 8073-53-8 | 29808 | NA 220 | Herbicide | Benzoic acid |
| Dicamba, aluminium salt | NA 1215 | ##### | NA 170 | Herbicide | Benzoic acid |
| Dicamba, butoxyethyl ester | NA 1477 | NA 220 | NA 219 | Herbicide | Benzoic acid |
| Dicamba, diethanolamine salt | 25059-78-3 | 29803 | 780 | Herbicide | Benzoic acid |
| Dicamba, diglycolamine salt | 104040-79-1 | ##### | 5007 | Herbicide | Benzoic acid |
| Dicamba, dimethylamine salt | 2300-66-5 | 29802 | 849 | Herbicide | Benzoic acid |
| Dicamba, dimethylamine salt, | NA 1123 | NA 165 | 90849 | Herbicide | Benzoic acid |
| other related | | | | | |
| Dicamba, isopropylamine salt | 55871-02-8 | ##### | 5098 | Herbicide | Benzoic acid |
| Dicamba, methyl ester | 6597-78-0 | ##### | NA 146 | Herbicide | Benzoic acid |
| Dicamba, monoethanolamine | 53404-28-7 | 29804 | 1829 | Herbicide | Benzoic acid |
| salt | | | | | |
| Dicamba, monoethanolamine | NA 1140 | NA 167 | 91829 | Herbicide | Benzoic acid |
| salt, other related | | | | | |
| Dicamba, other related | NA 1084 | NA 161 | 90200 | Herbicide | Benzoic acid |
| Dicamba, potassium salt | 10007-85-9 | ##### | 5110 | Herbicide | Benzoic acid |
| Dicamba, sodium salt | 1982-69-0 | 29806 | 5057 | Herbicide | Benzoic acid |
| Dicamba, triethanolamine salt | 53404-29-8 | 29805 | 1113 | Herbicide | Benzoic acid |
| Dichlorobenzoic acid | NA 1383 | NA 195 | NA 194 | Fungicide | Benzoic acid |
| methylester | | | | | |
| Diethanolamine 3-amino-2,5- | 53404-16-3 | 29904 | NA 222 | Herbicide | Benzoic acid |
| dichlorobenzoate | | | | | |
| Dodecylamine salicylate | 7491-21-6 | 39302 | 2003 | Insecticide, | Benzoic acid |
| | | | | Fungicide, | |
| | | | | Microbiocide | |
| Ethyl paraben | 120-47-8 | 61202 | 3204 | Fungicide, | Benzoic acid |
| | | | | Microbiocide | |
| Homosalate | 118-56-9 | 76603 | 3231 | Insecticide, | Benzoic acid |
| | | | | Fungicide, | |
| | | | | Microbiocide | |
| Methyl chloramben | 7286-84-2 | 29905 | NA 223 | Herbicide | Benzoic acid |
| Methyl paraben | 99-76-3 | 61201 | 1005 | Fungicide, | Benzoic acid |
| | | | | Microbiocide | |
| Methyl paraben, calcium salt | 40167-95-1 | 61208 | NA 499 | Fungicide, | Benzoic acid |
| | | | | Microbiocide | |
| Methyl paraben, sodium salt | 5026-62-0 | 61207 | NA 498 | Fungicide, | Benzoic acid |
| | | | | Microbiocide | |
| Monomethylammonium | 25182-03-0 | 29903 | NA 221 | Herbicide | Benzoic acid |
| chloramben (3-amino-2,5- | | | | | |
| dichlorobenzoic acid) | | | | | |
| Propyl paraben | 94-13-3 | 61203 | 2841 | Fungicide, | Benzoic acid |
| | | | | Microbiocide | |
| Propyl paraben, calcium salt | 83542-69-2 | 61206 | NA 497 | Fungicide, | Benzoic acid |
| | | | | Microbiocide | |
| Propyl paraben, sodium salt | 35285-69-9 | 61204 | NA 495 | Fungicide, | Benzoic acid |
| | | | | Microbiocide | |
| Salicylic acid | 69-72-7 | 76602 | 1170 | Insecticide, | Benzoic acid |
| | | | | Fungicide, | |
| | | | | Microbiocide | |
| Sodium 3-amino-2,5- | 1954-81-0 | 29906 | NA 224 | Herbicide | Benzoic acid |
| dichlorobenzoate | | | | | |
| Tricamba | 2307-49-5 | 17301 | NA 151 | Herbicide | Benzoic acid |
| Anisuron | 2689-43-2 | ##### | NA 138 | Herbicide | Benzoylurea |
| Diflubenzuron | 35367-38-5 | ##### | 1992 | Insecticide | Benzoylurea |
| Flucycloxuron | 113036-88-7 | ##### | NA 109 | Herbicide | Benzoylurea |
| Flucycloxuron, (E) | 94050-52-9 | NA 201 | NA 200 | Herbicide | Benzoylurea |
| Flufenoxuron | 101463-69-8 | ##### | NA 901 | Insecticide | Benzoylurea |
| Hexaflumuron | 86479-06-3 | ##### | 3899 | Insecticide | Benzoylurea |
| Lufenuron | 103055-07-8 | NA 184 | NA 183 | Insecticide | Benzoylurea |
| Novaluron | 116714-46-6 | NA 226 | NA 226 | Herbicide | Benzoylurea |
| Phenobenzuron | 449583 | ##### | NA 133 | Herbicide | Benzoylurea |
| Teflubenzuron | 83121-18-0 | ##### | NA 33 | Insecticide | Benzoylurea |
| Triflumuron | 64628-44-0 | ##### | 2961 | Insecticide | Benzoylurea |
| Diethamquat chloride | NA 1567 | NA 231 | NA 231 | Herbicide | Bipyridylium |
| Diquat bistribromide | 27041-82-3 | ##### | NA 146 | Herbicide | Bipyridylium |
| Diquat dibromide | 85-00-7 | 32201 | 229 | Herbicide, | Bipyridylium |
| | | | | Dessicant | |
| Diquat ion | 2764-72-9 | 32202 | NA 294 | N/A | Bipyridylium |
| Monolinuron with paraquat | 69312-67-0 | ##### | NA 119 | Herbicide | Urea, Bipyridylium |
| dichloride | | | | | |
| Morfamquat | 7411-47-4 | ##### | NA 135 | N/A | Bipyridylium |
| Morfamquat | 7411-47-4 | NA 201 | NA 200 | Herbicide | Bipyridylium |
| Morfamquat chloride | 4636-83-3 | NA 212 | NA 212 | Herbicide | Bipyridylium |
| Paraquat | 4685-14-7 | 61603 | NA 500 | Herbicide | Bipyridylium |
| Paraquat bis(methylsulfate) | 2074-50-2 | 61602 | 458 | Herbicide | Bipyridylium |
| Paraquat bistribromide | 27041-84-5 | ##### | NA 159 | Herbicide | Bipyridylium |
| Paraquat dichloride | 1910-42-5 | 61601 | 1601 | Herbicide | Bipyridylium |
| Priglone (diquat dibromide | 78403-23-3 | 32203 | NA 295 | N/A | Bipyridylium |
| w/paraquat ion) | | | | | |
| 1,3-Dimethyl-1,1,3,3- | 22232-20-8 | 34601 | NA 303 | N/A | Bis-Carbamate |
| disiloxanetetrol-1,3- | | | | | |
| bis(dimethylthiocarbamate) | | | | | |
| Desmedipham | 13684-56-5 | ##### | 1748 | Herbicide | Bis-Carbamate |
| Dimethyl (4-methyl-1,3- | 51543-98-7 | ##### | NA 888 | Fungicide | Bis-Carbamate |
| phenylenebis(iminocarbonyl- | | | | | |
| 1H-benzimidazole-1,2- | | | | | |
| diyl))biscarbamate | | | | | |
| Phenisopham | 57375-63-0 | NA 209 | NA 208 | Herbicide | Bis-Carbamate |
| Phenmedipham | 13684-63-4 | 98701 | 675 | Herbicide | Bis-Carbamate |
| Phenmedipham-ethyl | 13684-44-1 | ##### | NA 149 | Herbicide | Bis-Carbamate |
| 2-Hydroxy alachlor | NA 943 | NA 183 | 2602 | Breakdown | Chloroacetanilide |
| | | | | product | |
| Aatram (propachlor with | 8070-76-6 | 80816 | NA 781 | Herbicide | Triazine, |
| atrazine) (019101 + 080803) | | | | | Chloroacetanilide |
| Acetochlor | 34256-82-1 | ##### | 2349 | Herbicide | Chloroacetanilide |
| Alachlor | 15972-60-8 | 90501 | 678 | Herbicide | Chloroacetanilide |
| Butachlor | 23184-66-9 | ##### | 4056 | Herbicide | Chloroacetanilide |
| Butenachlor | 87310-56-3 | NA 206 | NA 206 | Herbicide | Chloroacetanilide |
| Delachlor | 24353-58-0 | ##### | NA 132 | Herbicide | Chloroacetanilide |
| Diethatyl-ethyl | 38727-55-8 | ##### | 1995 | Herbicide | Chloroacetanilide |
| Dimethachlor | 50563-36-5 | NA 184 | NA 183 | Herbicide | Chloroacetanilide |
| Metazachlor | 67129-08-2 | NA 184 | NA 183 | Herbicide | Chloroacetanilide |
| Metolachlor | 51218-45-2 | ##### | 1996 | Herbicide | Chloroacetanilide |
| Metolachlor, (S) | 87392-12-9 | ##### | 5133 | Herbicide | Chloroacetanilide |
| Pretilachlor | 51218-49-6 | NA 184 | NA 184 | Herbicide | Chloroacetanilide |
| Propachlor | 1918-16-7 | 19101 | 511 | Herbicide | Chloroacetanilide |
| Propisochlor | 86763-47-5 | NA 233 | NA 233 | Herbicide | Chloroacetanilide |
| Prynachlor | 21267-72-1 | ##### | NA 138 | Herbicide, | Chloroacetanilide |
| | | | | Plant Growth | |
| | | | | Regulator | |
| Thenylchlor | 96491-05-3 | NA 229 | NA 229 | Herbicide | Chloroacetanilide |
| (2-Methyl-4,6- | 13333-87-4 | ##### | NA 161 | Impurity | Chlorophenoxy acid |
| dichlorophenoxy)acetic acid | | | | | or ester |
| (as impurity) | | | | | |
| 2,3,6-Trichlorophenylacetic | 2903-64-2 | 82603 | NA 819 | Herbicide | Chlorophenoxy acid |
| acid | | | | | or ester |
| 2,3,6-Trichlorophenylacetic | 53404-91-4 | 82604 | NA 820 | Herbicide | Chlorophenoxy acid |
| acid, sodium salt | | | | | or ester |
| 2,4,5-T | 93-76-5 | 82001 | 639 | Herbicide | Chlorophenoxy acid |
| | | | | | or ester |
| 2,4,5-T tetradecylamine salt | 53535-37-8 | 82013 | 1989 | Herbicide | Chlorophenoxy acid |
| | | | | | or ester |
| 2,4,5-T, 2-ethylhexyl ester | 1928-47-8 | 82063 | 1092 | Herbicide | Chlorophenoxy acid |
| | | | | | or ester |
| 2,4,5-T, alkylamine salt | 53535-37-8 | 82013 | 818 | Herbicide | Chlorophenoxy acid |
| | | | | | or ester |
| 2,4,5-T, butoxyethanol ester | 2545-59-7 | 82053 | 819 | Herbicide | Chlorophenoxy acid |
| | | | | | or ester, Glycol Ether |
| 2,4,5-T, butoxyethanol ester | 2545-59-7 | 82072 | NA 166 | Herbicide | Chlorophenoxy acid |
| | | | | | or ester |
| 2,4,5-T, butoxypropyl ester | 1928-48-9 | 82055 | 820 | Herbicide | Chlorophenoxy acid |
| | | | | | or ester |
| 2,4,5-T, butyl ester | 93-79-8 | 82056 | 822 | Herbicide | Chlorophenoxy acid |
| | | | | | or ester |
| 2,4,5-T, butyric acid | 93-80-1 | ##### | NA 152 | Herbicide | Chlorophenoxy acid |
| | | | | | or ester |
| 2,4,5-T, dimethylamine salt | 6369-97-7 | 82019 | 1474 | Herbicide | Chlorophenoxy acid |
| | | | | | or ester |
| 2,4,5-T, dodecylamine salt | 53404-84-5 | 82011 | 1988 | Herbicide | Chlorophenoxy acid |
| | | | | | or ester |
| 2,4,5-T, isooctyl ester | 25168-15-4 | NA 121 | 851 | Herbicide | Chlorophenoxy acid |
| | | | | | or ester |
| 2,4,5-T, N-oleyl-1,3- | 53404-87-8 | 82029 | 1097 | Herbicide | Chlorophenoxy acid |
| propylenediamine salt | | | | | or ester |
| 2,4,5-T, propylene glycol butyl | 1928-48-9 | 82055 | 824 | Herbicide | Chlorophenoxy acid |
| ether ester | | | | | or ester |
| 2,4,5-T, triethylamine salt | 2008-46-0 | 82034 | 850 | Herbicide | Chlorophenoxy acid |
| | | | | | or ester |
| 2,4,5-trichlorophenoxyacetic | 69462-12-0 | 82064 | NA 799 | Herbicide | Chlorophenoxy acid |
| acid, 2-ethyl-4-methylpentyl | | | | | or ester |
| ester | | | | | |
| 2,4,5-trichlorophenoxyacetic | 53404-85-6 | 82012 | NA 790 | Herbicide | Chlorophenoxy acid |
| acid, alkyl(C13) amine salt | | | | | or ester |
| 2,4,5-trichlorophenoxyacetic | 120-39-8 | 82051 | NA 795 | Herbicide | Chlorophenoxy acid |
| acid, amyl ester | | | | | or ester |
| 2,4,5-trichlorophenoxyacetic | 1928-58-1 | 82052 | NA 796 | Herbicide | Chlorophenoxy acid |
| acid, butoxyethoxypropanol | | | | | or ester |
| ester | | | | | |
| 2,4,5-trichlorophenoxyacetic | 53404-86-7 | 82038 | NA 793 | Herbicide | Chlorophenoxy acid |
| acid, diethylethanolamine salt | | | | | or ester |
| 2,4,5-trichlorophenoxyacetic | 4938-72-1 | 82062 | NA 798 | Herbicide | Chlorophenoxy acid |
| acid, isobutyl ester | | | | | or ester |
| 2,4,5-trichlorophenoxyacetic | 93-78-7 | 82066 | NA 800 | Herbicide | Chlorophenoxy acid |
| acid, isopropyl ester | | | | | or ester |
| 2,4,5-trichlorophenoxyacetic | 55256-33-2 | 82039 | NA 794 | Herbicide | Chlorophenoxy acid |
| acid, N,N-dimethyl oleyl- | | | | | or ester |
| linoleyl amine salt | | | | | |
| 2,4,5-trichlorophenoxyacetic | 53404-88-9 | 82037 | NA 792 | Herbicide | Chlorophenoxy acid |
| acid, N,N- | | | | | or ester |
| dimethyllinoleylamine salt | | | | | |
| 2,4,5-trichlorophenoxyacetic | 53404-89-0 | 82020 | NA 791 | Herbicide | Chlorophenoxy acid |
| acid, N,N-dimethyloleylamine | | | | | or ester |
| salt | | | | | |
| 2,4,5-trichlorophenoxyacetic | 55491-33-3 | 82077 | NA 803 | Herbicide | Chlorophenoxy acid |
| acid, pentyl ester | | | | | or ester |
| 2,4,5-trichlorophenoxyacetic | 13560-99-1 | 82004 | NA 789 | Herbicide | Chlorophenoxy acid |
| acid, sodium salt | | | | | or ester |
| 2,4,5-trichlorophenoxyacetic | 53535-32-3 | 82075 | NA 802 | Herbicide | Chlorophenoxy acid |
| acid, tripropylene glycol | | | | | or ester |
| isobutyl ether ester | | | | | |
| 2,4-D | 94-75-7 | 30001 | 636 | Herbicide, | Chlorophenoxy acid |
| | | | | Plant Growth | or ester |
| | | | | Regulator | |
| 2,4-D with 2,4,5-T | 8015-35-8 | 30077 | NA 259 | Herbicide | Chlorophenoxy acid |
| | | | | | or ester |
| 2,4-D, 2-ethyl-4-methylpentyl | 53404-37-8 | NA 227 | NA 227 | Herbicide | Chlorophenoxy acid |
| ester | | | | | or ester |
| 2,4-D, 2-ethylhexyl ester | 1928-43-4 | 30063 | 1622 | Herbicide | Chlorophenoxy acid |
| | | | | | or ester |
| 2,4-D, alkanolamine salts | 2307-55-3 | 30005 | 801 | Herbicide | Chlorophenoxy acid |
| (ethanol and isopropanol | | | | | or ester |
| amines) | | | | | |
| 2,4-D, alkyl (C12) amine salts | 2212-54-6 | 30011 | 980 | Herbicide | Chlorophenoxy acid |
| | | | | | or ester |
| 2,4-D, alkyl (C14) amine salts | 28685-18-9 | 30013 | 981 | Herbicide | Chlorophenoxy acid |
| | | | | | or ester |
| 2,4-D, butoxy ethoxy propanol | 1928-57-0 | 30052 | 1255 | Herbicide | Chlorophenoxy acid |
| ester | | | | | or ester |
| 2,4-D, butoxyethanol ester | 1929-73-3 | 30053 | 802 | Herbicide | Chlorophenoxy acid |
| | | | | | or ester, Glycol Ether |
| 2,4-D, butoxyethanol ester | 1929-73-3 | 30061 | NA 166 | Herbicide | Chlorophenoxy acid |
| | | | | | or ester |
| 2,4-D, butoxypropyl ester | 1928-45-6 | 30055 | 803 | Herbicide | Chlorophenoxy acid |
| | | | | | or ester |
| 2,4-D, butyl ester | 94-80-4 | 30056 | 804 | Herbicide | Chlorophenoxy acid |
| | | | | | or ester |
| 2,4-D, chlorocrotyl ester | 2971-38-2 | NA 228 | NA 227 | Herbicide | Chlorophenoxy acid |
| | | | | | or ester |
| 2,4-D, diethanolamine salt | 5742-19-8 | 30016 | 805 | Herbicide | Chlorophenoxy acid |
| | | | | | or ester |
| 2,4-D, diethylamine salt | 20940-37-8 | 30017 | 875 | Herbicide | Chlorophenoxy acid |
| | | | | | or ester |
| 2,4-D, dimethylamine salt | 2008-39-1 | 30019 | 806 | Herbicide | Chlorophenoxy acid |
| | | | | | or ester |
| 2,4-D, dodecylamine salt | 2212-54-6 | 30011 | 807 | Herbicide | Chlorophenoxy acid |
| | | | | | or ester |
| 2,4-D, heptylamine salt | 37102-63-9 | 30023 | 1962 | Herbicide | Chlorophenoxy acid |
| | | | | | or ester |
| 2,4-D, isooctyl ester | 25168-26-7 | 30064 | 809 | Herbicide | Chlorophenoxy acid |
| | | | | | or ester |
| Triclopyr | 55335-06-3 | ##### | NA 36 | Herbicide | Chloropyridinyl |
| Triclopyr ethyl ester | 60825-27-6 | ##### | NA 108 | Herbicide | Chloropyridinyl |
| Triclopyr, butoxyethyl ester | 64700-56-7 | ##### | 2170 | Herbicide | Chloropyridinyl, |
| | | | | | Glycol Ether |
| Triclopyr, triethylamine salt | 57213-69-1 | ##### | 2131 | Herbicide | Chloropyridinyl |
| Alloxydim | 55634-91-8 | NA 203 | NA 203 | Herbicide | Cyclohexenone |
| | | | | | derivative |
| Alloxydim sodium | 55635-13-7 | ##### | NA 926 | Herbicide | Cyclohexenone |
| | | | | | derivative |
| Butroxydim | 138164-12-2 | NA 202 | NA 202 | Herbicide | Cyclohexenone |
| | | | | | derivative |
| Clethodim | 99129-21-2 | ##### | 3566 | Herbicide | Cyclohexenone |
| | | | | | derivative |
| Cloproxydim | 95480-33-4 | NA 231 | NA 231 | Herbicide | Cyclohexenone |
| | | | | | derivative |
| Cycloxydim | 101205-02-1 | NA 184 | NA 183 | Herbicide | Cyclohexenone |
| | | | | | derivative |
| Sethoxydim | 74051-80-2 | ##### | 2177 | Herbicide | Cyclohexenone |
| | | | | | derivative |
| Tepraloxydim | 149979-41-9 | ##### | NA 982 | Herbicide | Cyclohexenone |
| | | | | | derivative |
| Tralkoxydim | 87820-88-0 | ##### | 5457 | Herbicide | Cyclohexenone |
| | | | | | derivative |
| 2,4-dinitrophenol | 51-28-5 | 37509 | 221 | Impurity | Dinitrophenol |
| | | | | | derivative |
| 2,4-dinitrophenol, sodium salt | 1011-73-0 | ##### | NA 161 | Impurity, | Dinitrophenol |
| (as impurity) | | | | Breakdown | derivative |
| | | | | product | |
| 2,4-Dinitrophenyl thiocyanate | 1594-56-5 | ##### | NA 146 | N/A | Dinitrophenol |
| | | | | | derivative |
| 2,6-dinitrophenol, sodium salt | 32581-06-9 | ##### | NA 161 | Impurity | Dinitrophenol |
| (as impurity) | | | | | derivative |
| 2-Cyclohexyl-4,6-dinitrophenol | 317-83-9 | 37502 | NA 316 | N/A | Dinitrophenol |
| dicyclohexylamine | | | | | derivative |
| 3,3′-Dichloro-5,5′-dinitro-(1,1′- | 15595-24-1 | ##### | NA 137 | N/A | Dinitrophenol |
| biphenyl)-2,2′-diol | | | | | derivative |
| 3,3′-Dichloro-5,5′-dinitro-(1,1′- | 63992-31-4 | ##### | NA 137 | N/A | Dinitrophenol |
| biphenyl)-2,2′-diol | | | | | derivative |
| 4,6-Dinitro-2- | 69632-97-9 | ##### | NA 145 | N/A | Dinitrophenol |
| cyclohexylphenol, 2-((2- | | | | | derivative |
| aminoethyl)amino)ethanol salt | | | | | |
| 4,6-Dinitro-2- | 69632-98-0 | ##### | NA 145 | N/A | Dinitrophenol |
| cyclohexylphenol, | | | | | derivative |
| triethanolamine salt | | | | | |
| 5,5′-Dichloro-3,3′-dinitro-(1,1′- | 10331-57-4 | ##### | NA 137 | N/A | Dinitrophenol |
| biphenyl)-2,2′-diol | | | | | derivative |
| 6,6′-Dichloro-4,4′-dinitro-(1,1′- | 21832-25-7 | ##### | NA 137 | N/A | Dinitrophenol |
| biphenyl)-2,2′-diol | | | | | derivative |
| Binapacryl | 485-31-4 | 12201 | 73 | Herbicide | Dinitrophenol |
| | | | | | derivative |
| Diethanolamine dinoseb (2- | 53404-43-6 | 37514 | NA 320 | Herbicide | Dinitrophenol |
| sec-butyl-4,6-dinitrophenol) | | | | | derivative |
| Dinex | 131-89-5 | 37501 | NA 315 | N/A | Dinitrophenol |
| | | | | | derivative |
| Dinitro cresol | 534-52-1 | 37507 | 3170 | Fungicide | Dinitrophenol |
| | | | | | derivative |
| Dinitro-1-methyl heptyl phenol | NA 159 | NA 302 | 758 | N/A | Dinitrophenol |
| | | | | | derivative |
| Dinitrophenol (mixed isomers) | 25550-58-7 | NA 223 | NA 223 | Breakdown | Dinitrophenol |
| | | | | product, | derivative |
| | | | | Impurity | |
| Dinobuton | 973-21-7 | ##### | 2526 | Insecticide, | Dinitrophenol |
| | | | | Fungicide | derivative |
| Dinocap | 39300-45-3 | 36001 | 344 | Fungicide, | Dinitrophenol |
| | | | | Insecticide | derivative |
| Dinocap, other related | NA 1091 | NA 162 | 90344 | Insecticide, | Dinitrophenol |
| | | | | Fungicide | derivative |
| Dinocton | 32534-96-6 | NA 207 | NA 206 | Insecticide, | Dinitrophenol |
| | | | | Fungicide | derivative |
| Dinofenate | 61614-62-8 | ##### | NA 146 | Herbicide | Dinitrophenol |
| | | | | | derivative |
| Dinopenton | 5386-57-2 | ##### | NA 154 | N/A | Dinitrophenol |
| | | | | | derivative |
| Dinoprop | 7257-41-2 | ##### | NA 138 | N/A | Dinitrophenol |
| | | | | | derivative |
| Dinosam | 4097-36-3 | 37504 | NA 318 | N/A | Dinitrophenol |
| | | | | | derivative |
| Dinoseb | 88-85-7 | 37505 | 238 | Herbicide, | Dinitrophenol |
| | | | | Defoliant | derivative |
| Dinoseb acetate | 2813-95-8 | NA 207 | NA 206 | Herbicide | Dinitrophenol |
| | | | | | derivative |
| Dinoseb, amine salt | 2244387 | 37511 | 239 | Herbicide, | Dinitrophenol |
| | | | | Defoliant | derivative |
| Dinoseb, ammonium salt | 6365-83-9 | 37513 | 240 | Herbicide, | Dinitrophenol |
| | | | | Defoliant | derivative |
| Dinoseb, sodium salt | 35040-03-0 | 37512 | 1445 | Herbicide, | Dinitrophenol |
| | | | | Defoliant | derivative |
| Dinoseb, triethanolamine salt | 6420-47-9 | 37506 | 235 | Herbicide, | Dinitrophenol |
| | | | | Defoliant | derivative |
| Dinosulfon | 5386-77-6 | ##### | NA 157 | N/A | Dinitrophenol |
| | | | | | derivative |
| Dinoterb | 1420-07-1 | ##### | NA 128 | Herbicide | Dinitrophenol |
| | | | | | derivative |
| Dinoterbon | 6073-72-9 | ##### | NA 128 | Herbicide | Dinitrophenol |
| | | | | | derivative |
| DNOC, sodium salt | 2312-76-7 | 37508 | 533 | Fungicide, | Dinitrophenol |
| | | | | Microbiocide, | derivative |
| | | | | Insecticide, | |
| | | | | Herbicide | |
| Etinofen | 2544-94-7 | ##### | NA 147 | N/A | Dinitrophenol |
| | | | | | derivative |
| Medinoterb acetate | 212943 | NA 208 | NA 208 | Herbicide | Dinitrophenol |
| | | | | | derivative |
| Methyl 2-(1-methylheptyl)-4,6- | 5386-68-5 | ##### | NA 157 | N/A | Dinitrophenol |
| dinitrophenyl carbonate | | | | | derivative |
| Premerge plus (naptalam with | 8075-57-8 | 30704 | NA 278 | Herbicide | Dinitrophenol |
| dinoseb) | | | | | derivative |
| Sodium naptalam with sodium | 59915-53-6 | 30705 | NA 279 | Herbicide | Dinitrophenol |
| dinoseb | | | | | derivative |
| Sultropen | 963-22-4 | ##### | NA 146 | N/A | Dinitrophenol |
| | | | | | derivative |
| Acifluorfen | 50594-66-6 | ##### | NA 935 | Herbicide | Diphenyl ether |
| Acifluorfen, sodium salt | 62476-59-9 | ##### | 2218 | Herbicide | Diphenyl ether |
| Aclonifen | 74070-46-5 | NA 187 | NA 187 | Herbicide | Diphenyl ether |
| Bifenox | 42576-02-3 | ##### | 1953 | Herbicide | Diphenyl ether |
| Chlomethoxyfen | 32861-85-1 | NA 185 | NA 185 | Herbicide | Diphenyl ether |
| Diofenolan | 63837-33-2 | 7703 | NA 101 | Herbicide | Diphenyl ether |
| Ethyl acifluorfen | 77207-01-3 | ##### | NA 936 | Herbicide | Diphenyl ether |
| Fluoroglycofen | 77501-60-1 | NA 181 | NA 180 | Herbicide | Diphenyl ether |
| Fomesafen | 72178-02-0 | ##### | NA 17 | Herbicide | Diphenyl ether |
| Fomesafen sodium | 108731-70-0 | ##### | 5086 | Herbicide | Diphenyl ether |
| Lactofen | 77501-63-4 | ##### | 3538 | Herbicide | Diphenyl ether |
| Methyl acifluorfen | 50594-67-7 | ##### | NA 937 | Herbicide | Diphenyl ether |
| Oxyfluorfen | 42874-03-3 | ##### | 1973 | Herbicide | Diphenyl ether |
| Bromobonil | 25671-46-9 | ##### | NA 134 | Herbicide | Hydroxybenzonitrile |
| Bromoxynil butyrate | 3861-41-4 | 35303 | 2163 | Herbicide | Hydroxybenzonitrile |
| Bromoxynil heptanoate | 56634-95-8 | ##### | 5036 | Herbicide | Hydroxybenzonitrile |
| Bromoxynil octanoate | 1689-99-2 | 35302 | 834 | Herbicide | Hydroxybenzonitrile |
| Bromoxynil pentanoate | NA 1476 | NA 220 | NA 219 | Herbicide | Hydroxybenzonitrile |
| Bromoxynil phenol | 1689-84-5 | 35301 | 2429 | Herbicide | Hydroxybenzonitrile |
| Chloroxynil | 1891-95-8 | ##### | NA 138 | Herbicide | Hydroxybenzonitrile |
| Ioxynil | 1689-83-4 | ##### | 2616 | Herbicide | Hydroxybenzonitrile |
| Ioxynil lithium | 2961-61-7 | ##### | NA 141 | Herbicide | Hydroxybenzonitrile |
| Ioxynil octanoate | 3861-47-0 | ##### | NA 141 | Herbicide | Hydroxybenzonitrile |
| Ioxynil sodium | 2961-62-8 | ##### | NA 141 | Herbicide | Hydroxybenzonitrile |
| 3-5-(1,1-Dimethylethyl)-3- | 78327-32-9 | ##### | NA 108 | Algaecide | Imidazolinone |
| isoxazolyl-4-hydroxy-1- | | | | | |
| methyl-2-imidazolidinone | | | | | |
| 3-Pyridinecarboxylic acid, 2- | 81334-60-3 | ##### | NA 1 | Herbicide | Imidazolinone |
| (4,5-dihydro-4-methyl-4-(1- | | | | | |
| methylethyl)-5-oxo-1H- | | | | | |
| imidazol-2-yl)-5-methyl-, (.+−.)- | | | | | |
| Imazamethabenz | 81405-85-8 | ##### | 2240 | Herbicide | Imidazolinone |
| Imazamox | 114311-32-9 | ##### | NA 2 | Herbicide | Imidazolinone |
| Imazapic | 104098-48-8 | NA 212 | NA 211 | Herbicide | Imidazolinone |
| Imazapyr | 81334-34-1 | ##### | 2256 | Herbicide | Imidazolinone |
| Imazapyr, isopropylamine salt | 81510-83-0 | ##### | 2257 | Herbicide | Imidazolinone |
| Imazaquin | 81335-37-7 | ##### | 2613 | Herbicide, | Imidazolinone |
| | | | | Plant Growth | |
| | | | | Regulator | |
| Imazaquin, ammonium salt | 81335-47-9 | ##### | 3006 | Herbicide | Imidazolinone |
| Imazaquin, sodium salt | 81335-46-8 | ##### | 5109 | Herbicide | Imidazolinone |
| Imazethapyr | 81335-77-5 | ##### | 2340 | Herbicide, | Imidazolinone |
| | | | | Plant Growth | |
| | | | | Regulator | |
| Imazethapyr, ammonium salt | 101917-66-2 | ##### | 2341 | Herbicide | Imidazolinone |
| Pyridine carboxylic acid, 2- | 104098-49-9 | ##### | 5097 | Herbicide | Imidazolinone |
| (4,5-dihydro-4-methyl-4-(1- | | | | | |
| methylethyl)-5-oxo-1H- | | | | | |
| imidazol-2-yl)-5-methyl, | | | | | |
| monoammonium salt | | | | | |
| Chloreturon | 20782-58-5 | NA 231 | NA 230 | Herbicide | Phenylurea |
| Daimuron | 42609-52-9 | NA 204 | NA 204 | Herbicide | Phenylurea |
| Fluothiuron | 33439-45-1 | NA 232 | NA 232 | Herbicide | Phenylurea |
| Methyldymron | 42609-73-4 | NA 205 | NA 204 | Herbicide | Phenylurea |
| Tetrafluoron | 27954-37-6 | ##### | NA 145 | Herbicide | Phenylurea |
| Bilanafos | 71048-99-2 | NA 202 | NA 201 | Herbicide | Phosphinico amino |
| | | | | | acid |
| Ampropylofos | 16606-64-7 | NA 181 | NA 180 | Fungicide | Phosphonic acid |
| Glyphosate | 1071-83-6 | ##### | 2997 | Herbicide | Phosphonoglycine |
| Glyphosate, ammonium salt | 114370-14-8 | NA 228 | 2319 | Herbicide | Phosphonoglycine |
| Glyphosate, diammonium salt | NA 1566 | NA 229 | 5810 | Herbicide | Phosphonoglycine |
| Glyphosate, isopropylamine | 38641-94-0 | ##### | 1855 | Herbicide | Phosphonoglycine |
| salt | | | | | |
| Glyphosate, monoammonium | 114370-14-8 | ##### | 2301 | Herbicide | Phosphonoglycine |
| salt | | | | | |
| Glyphosate, sodium sesqui salt | 70393-85-0 | ##### | 2275 | Herbicide | Phosphonoglycine |
| Glyphosate-trimesium | 81591-81-3 | ##### | 2327 | Herbicide | Phosphonoglycine |
| N,N- | 2439-99-8 | ##### | NA 884 | Herbicide | Phosphonoglycine |
| Bis(phosphonomethyl)glycine | | | | | |
| 6-chloropicolinic acid | 4684-94-0 | 69206 | NA 657 | Herbicide | Pyridinecarboxylic |
| | | | | | acid |
| Clopyralid | 1702-17-6 | ##### | 5135 | Herbicide | Pyridinecarboxylic |
| | | | | | acid |
| Clopyralid copper | NA 1458 | NA 216 | NA 216 | Herbicide | Pyridinecarboxylic |
| | | | | | acid |
| Clopyralid, monoethanolamine | 57754-85-5 | ##### | 5050 | Herbicide | Pyridinecarboxylic |
| salt | | | | | acid |
| Clopyralid, triethylamine salt | NA 955 | ##### | 2339 | Herbicide | Pyridinecarboxylic |
| | | | | | acid |
| Picloram | 5145 | 5101 | 593 | Herbicide | Pyridinecarboxylic |
| | | | | | acid |
| Picloram, alkanolamine salt | NA 1479 | NA 220 | NA 219 | Herbicide | Pyridinecarboxylic |
| | | | | | acid |
| Picloram, diethanolamine salt | 5145 | NA 220 | NA 220 | Herbicide | Pyridinecarboxylic |
| | | | | | acid |
| Picloram, isooctyl ester | 26952-20-5 | 5103 | 835 | Herbicide | Pyridinecarboxylic |
| | | | | | acid |
| Picloram, potassium salt | 2545-60-0 | 5104 | 5330 | Herbicide | Pyridinecarboxylic |
| | | | | | acid |
| Picloram, triethylamine salt | 35832-11-2 | 5105 | NA 77 | Herbicide | Pyridinecarboxylic |
| | | | | | acid |
| Picloram, triisopropanolamine | 6753-47-5 | 5102 | 1099 | Herbicide | Pyridinecarboxylic |
| salt | | | | | acid |
| Picolinafen | 137641-05-5 | NA 210 | NA 210 | Herbicide | Pyridinecarboxylic |
| | | | | | acid |
| Thiazopyr | 117718-60-2 | ##### | 3984 | Herbicide | Pyridinecarboxylic |
| | | | | | acid |
| Amidosulfuron | 120923-37-7 | NA 183 | NA 183 | Herbicide | Sulfonylurea |
| Azimsulfuron | 120162-55-2 | ##### | NA 112 | Herbicide | Sulfonylurea |
| Bensulfuron | 99283-01-9 | NA 181 | NA 180 | Herbicide | Sulfonylurea |
| Bensulfuron methyl | 83055-99-6 | ##### | 2263 | Herbicide | Sulfonylurea |
| Chlorimuron | 99283-00-8 | NA 204 | NA 203 | Herbicide | Sulfonylurea |
| Chlorimuron ethyl | 90982-32-4 | ##### | 2458 | Herbicide | Sulfonylurea |
| Chlorsulfuron | 64902-72-3 | ##### | 2143 | Herbicide | Sulfonylurea |
| Cinosulfuron | 94593-91-6 | NA 183 | NA 183 | Herbicide | Sulfonylurea |
| Cyclosulfamuron | 136849-15-5 | NA 204 | NA 203 | Herbicide | Sulfonylurea |
| Ethametsulfuron | 111353-84-5 | NA 212 | NA 212 | Herbicide | Sulfonylurea |
| Ethametsulfuron-methyl | 97780-06-8 | ##### | NA 43 | Herbicide | Sulfonylurea |
| Ethoxysulfuron | 126801-58-9 | NA 226 | NA 225 | Herbicide | Sulfonylurea |
| Flazasulfuron | 104040-78-0 | NA 229 | NA 228 | Herbicide | Sulfonylurea |
| Flupyrsulfuron | 150315-10-9 | NA 205 | NA 204 | Herbicide | Sulfonylurea |
| Flupyrsulfuron-methyl | NA 1460 | NA 216 | NA 216 | Herbicide | Sulfonylurea |
| Flupyrsulfuron-methyl, sodium | 144740-54-5 | NA 218 | NA 217 | Herbicide | Sulfonylurea |
| salt | | | | | |
| Halosulfuron | 100784-20-1 | ##### | 3919 | Herbicide | Sulfonylurea |
| Imazosulfuron | 122548-33-8 | NA 229 | NA 229 | Herbicide | Sulfonylurea |
| Iodosulfuron methyl | 185119-76-0 | NA 212 | NA 211 | Herbicide | Sulfonylurea |
| Iodosulfuron methyl, sodium | 144550-36-7 | NA 212 | NA 211 | Herbicide | Sulfonylurea |
| salt | | | | | |
| Mesosulfuron-methyl | 208465-21-8 | ##### | NA 993 | Herbicide | Sulfonylurea |
| Metsulfuron | 5585-64-8 | NA 183 | NA 182 | Herbicide | Sulfonylurea |
| Metsulfuron-methyl | 74223-64-6 | ##### | 2222 | Herbicide | Sulfonylurea |
| Nicosulfuron | 111991-09-4 | ##### | 3829 | Herbicide | Sulfonylurea |
| Oxasulfuron | 144651-06-9 | ##### | NA 991 | Herbicide | Sulfonylurea |
| Primisulfuron-methyl | 86209-51-0 | ##### | 5103 | Herbicide | Sulfonylurea |
| Prosulfuron | 94125-34-5 | ##### | 5115 | Herbicide | Sulfonylurea |
| Pyrazosulfuron | 98389-04-9 | NA 205 | NA 205 | Herbicide | Sulfonylurea |
| Pyrazosulfuron-ethyl | 93697-74-6 | NA 218 | NA 217 | Herbicide | Sulfonylurea |
| Rimsulfuron | 122931-48-0 | ##### | 3835 | Herbicide | Sulfonylurea |
| Sulfometuron | 74223-56-6 | NA 206 | NA 205 | Herbicide | Sulfonylurea |
| Sulfometuron methyl | 74222-97-2 | ##### | 2149 | Herbicide | Sulfonylurea |
| Sulfosulfuron | 141776-32-1 | 85601 | 5136 | Herbicide | Sulfonylurea |
| Thifensulfuron | 79277-67-1 | NA 183 | NA 182 | Herbicide | Sulfonylurea |
| Thifensulfuron-methyl | 79277-27-3 | ##### | 2237 | Herbicide | Sulfonylurea |
| Triasulfuron | 82097-50-5 | ##### | 5100 | Herbicide | Sulfonylurea |
| Tribenuron | 106040-48-6 | NA 206 | NA 205 | Herbicide | Sulfonylurea |
| Tribenuron methyl | 101200-48-0 | ##### | 2338 | Herbicide | Sulfonylurea |
| Triflusulfuron-methyl | 126535-15-7 | ##### | 3875 | Herbicide | Sulfonylurea |
| Tritosulfuron | 142469-14-5 | NA 234 | NA 234 | Herbicide | Sulfonylurea |
| Butylate | 2008-41-5 | 41405 | 565 | Herbicide | Thiocarbamate |
| Calcium thylenebis | 5895-18-1 | 14501 | NA 139 | Microbiocide | Dithiocarbamate |
| (dithiocarbamate) | | | | | |
| Cufraneb | 11096-18-7 | NA 181 | NA 180 | Fungicide | Dithiocarbamate |
| Cycloate | 1134-23-2 | 41301 | 516 | Herbicide | Thiocarbamate |
| Diammonium | 607313 | 14502 | NA 140 | Microbiocide | Dithiocarbamate |
| ethylenebis(dithiocarbamate) | | | | | |
| Dimepiperate | 61432-55-1 | NA 186 | NA 185 | Herbicide | Thiocarbamate |
| EPTC | 759-94-4 | 41401 | 264 | Herbicide | Thiocarbamate |
| Esprocarb | 85785-20-2 | NA 203 | NA 202 | Herbicide | Thiocarbamate |
| Ethiolate | 2941-55-1 | ##### | 1793 | Herbicide | Thiocarbamate |
| Fenothiocarb | 62850-32-2 | NA 188 | NA 187 | Insecticide | Thiocarbamate |
| Ferbam | 14484-64-1 | 34801 | 288 | Fungicide | Dithiocarbamate |
| Ferric nitroso dimethyl | 83542-83-0 | ##### | NA 149 | Microbiocide | Dithiocarbamate |
| dithiocarbamate | | | | | |
| Furathiocarb | 65907-30-4 | NA 176 | NA 52 | Insecticide | Thiocarbamate |
| Isopolinate | 3134-70-1 | NA 232 | NA 232 | Herbicide | Thiocarbamate |
| Mancopper | 53988-93-5 | NA 181 | NA 180 | Fungicide | Dithiocarbamate |
| Mancozeb | 2233100 | 14504 | 211 | Fungicide | Dithiocarbamate, |
| | | | | | Inorganic-Zinc |
| Maneb | 12427-38-2 | 14505 | 369 | Fungicide | Dithiocarbamate |
| Maneb and nickel sulfate | 8005-46-7 | ##### | NA 158 | Fungicide | Dithiocarbamate, |
| hexahydrate (014505 + 050505) | | | | | Inorganic-Nickel |
| Manganous | 15339-36-3 | 34802 | NA 304 | Fungicide | Dithiocarbamate |
| dimethyldithiocarbamate | | | | | |
| Mercuric dimethyl | 15415-64-2 | 34808 | 709 | Microbiocide | Inorganic-Mercury, |
| dithiocarbamate | | | | | Heavy metal, |
| | | | | | Dithiocarbamate |
| Metam sodium, dihydrate | 137-42-8 | 39003 | NA 163 | Fumigant, | Dithiocarbamate |
| | | | | Fungicide, | |
| | | | | Microbiocide, | |
| | | | | Algaecide, | |
| | | | | Nematicide | |
| Metam-sodium | 6734-80-1 | 39003 | 616 | Fumigant, | Dithiocarbamate |
| | | | | Herbicide, | |
| | | | | Fungicide, | |
| | | | | Microbiocide, | |
| | | | | Algaecide, | |
| | | | | Nematicide | |
| Methasulfocarb | 66952-49-6 | NA 202 | NA 201 | Fungicide, | Thiocarbamate |
| | | | | Plant Growth | |
| | | | | Regulator | |
| Methiobencarb | 18357-78-3 | NA 232 | NA 232 | Herbicide | Thiocarbamate |
| Metiram | 9006-42-2 | 14601 | 493 | Fungicide | Dithiocarbamate, |
| | | | | | Inorganic-Zinc |
| Molinate | 2212-67-1 | 41402 | 449 | Herbicide | Thiocarbamate |
| Molinate sulfoxide | NA 1052 | NA 156 | 4080 | Breakdown | Thiocarbamate |
| | | | | product | |
| Morpholinomethyl | 31848-11-0 | ##### | NA 158 | Microbiocide | Dithiocarbamate |
| dimethyldithiocarbamate | | | | | |
| Nabam | 142-59-6 | 14503 | 417 | Fungicide, | Dithiocarbamate |
| | | | | Herbicide | |
| Orbencarb | 34622-58-7 | NA 189 | NA 188 | Herbicide | Thiocarbamate |
| Pebulate | 1114-71-2 | 41403 | 590 | Herbicide | Thiocarbamate |
| Phenylmercuric | 32407-99-1 | 66008 | NA 555 | Microbiocide, | Organomercury, |
| dimethyldithiocarbamate | | | | Fungicide | Dithiocarbamate, |
| | | | | | Heavy metal |
| Potassium ammonium | 22221-14-3 | 14507 | NA 141 | Microbiocide | Dithiocarbanaate |
| ethylenebis(dithiocarbamate) | | | | | |
| Potassium dimethyl dithiocarbamate | 128-03-0 | 34803 | 1934 | Microbiocide | Dithiocarbamate |
| Potassium N-hydroxymethyl- | 51026-28-9 | ##### | 1824 | N/A | Dithiocarbamate |
| N-methyldithio carbamate | | | | | |
| Potassium N-methyldithiocarbamate | 137-41-7 | 39002 | 970 | Fumigant, | Dithiocarbamate |
| | | | | Fungicide, | |
| | | | | Microbiocide, | |
| | | | | Algaecide, | |
| | | | | Nematicide | |
| Propineb | 12071-83-9 | ##### | NA 155 | Fungicide, | Dithiocarbamate, |
| | | | | Microbiocide | Inorganic-Zinc |
| Prosulfocarb | 52888-80-9 | NA 185 | NA 184 | Herbicide | Thiocarbamate |
| Prothiocarb | 19622-08-3 | NA 183 | NA 182 | Fungicide | Thiocarbamate |
| Prothiocarb hydrochloride | 19622-19-6 | NA 200 | NA 199 | Fungicide | Thiocarbamate |
| Pyributicarb | 88678-67-5 | NA 229 | NA 229 | Herbicide | Thiocarbamate |
| S-tert-Butyl | 2212-63-7 | ##### | NA 128 | Herbicide | Thiocarbamate |
| dipropylthiocarbamate | | | | | |
| Sodium dimethyl dithiocarbamate | 128-04-1 | 34804 | 548 | Fungicide | Dithiocarbamate |
| Sulfallate | 95-06-7 | 39001 | 115 | Herbicide | Dithiocarbamate |
| Tert-butyl dimethyl trithioperoxycarbamate | 3304-97-0 | 34807 | 1383 | Rodent | Dithiocarbamate |
| | | | | Repellent | |
| Tert- | NA 1130 | NA 166 | 91383 | N/A | Dithiocarbamate |
| butyldimethyltrithioperoxycarbamate, | | | | | |
| other related | | | | | |
| Thiobencarb | 28249-77-6 | ##### | 1933 | Herbicide | Thiocarbamate |
| Thiobencarb sulfoxide | NA 1042 | NA 139 | 2937 | Breakdown | Thiocarbamate |
| | | | | product | |
| Thiodicarb | 59669-26-0 | ##### | 2202 | Molluscicide, | Thiocarbamate |
| | | | | Insecticide | |
| Thiram | 137-26-8 | 79801 | 589 | Fungicide | Dithiocarbamate |
| Thiram and NAD and IBA and | 75497-92-6 | 56011 | NA 463 | Fungicide, | Dithiocarbamate |
| 2-methyl-1- | | | | Plant Growth | |
| naphthaleneacetamide and 2- | | | | Regulator | |
| methyl-1-naphthaleneacetic | | | | | |
| acid | | | | | |
| Tiocarbazil | 36756-79-3 | ##### | NA 911 | Herbicide | Thiocarbamate |
| Triallate | 2303-17-5 | 78802 | 49 | Herbicide | Thiocarbamate |
| Tricopper dichloride | 7076-63-3 | ##### | NA 152 | Microbiocide | Dithiocarbamate, |
| dimethyldithiocarbamate | | | | | Inorganic-Copper |
| Vernolate | 1929-77-7 | 41404 | 1987 | Herbicide | Thiocarbamate |
| Zineb | 12122-67-7 | 14506 | 627 | Fungicide | Dithiocarbamate, |
| | | | | | Inorganic-Zinc |
| Zineb-ethylene thiuram | NA 1465 | NA 217 | NA 216 | Fungicide | Dithiocarbamate, |
| disulfide adduct | | | | | Inorganic-Zinc |
| Ziram | 137-30-4 | 34805 | 629 | Fungicide, | Dithiocarbamate, |
| | | | | Microbiocide, | Inorganic-Zinc |
| | | | | Dog and Cat | |
| | | | | Repellent | |
| Ziram, cyclohexylamine | 16509-79-8 | 34806 | 1328 | Dog and Cat | Dithiocarbamate, |
| complex | | | | Repellent, | Inorganic-Zinc |
| | | | | Fungicide | |
| 1,3,5-Triethylhexahydro-s- | 7779-27-3 | 82901 | NA 823 | Fungicide | Triazine |
| triazine | | | | | |
| 2-(Diethylamino)-4- | 3004-70-4 | 80812 | NA 780 | N/A | Triazine |
| (isopropylamino)-6-methoxy-s- | | | | | |
| triazine | | | | | |
| 2-chloro-4- | 29450-57-5 | ##### | NA 135 | N/A | Triazine |
| ((hydroxymethyl)amino)-6- | | | | | |
| (isopropylamino)-s-triazine | | | | | |
| 3-(chloromethyl)-4-ketobenz- | 24310-41-6 | ##### | NA 162 | Impurity | Triazine |
| 1,2,3-triazine (as impurity) | | | | | |
| 3-(Hydroxymethyl)-4- | 24310-40-5 | ##### | NA 162 | Impurity | Triazine |
| ketobenz-1,2,3-triazine (as | | | | | |
| impurity) | | | | | |
| Aatram (propachlor with | 8070-76-6 | 80816 | NA 781 | Herbicide | Triazine, |
| atrazine) (019101 + 080803) | | | | | Chloroacetanilide |
| Ametryne | 834-12-8 | 80801 | 18 | Herbicide | Triazine |
| Ametryne, other related | NA 1071 | NA 160 | 90018 | Herbicide | Triazine |
| Anilazine | 101-05-3 | 80811 | 256 | Fungicide | Triazine |
| Atraton | 1610-17-9 | 80802 | 4050 | Herbicide | Triazine |
| Atrazine | 1912-24-9 | 80803 | 45 | Herbicide | Triazine |
| Atrazine dealkylated | NA 1059 | NA 157 | 4052 | Breakdown | Triazine |
| | | | | product | |
| Atrazine, other related | NA 1072 | NA 160 | 90045 | Herbicide | Triazine |
| Aziprotryne | 4658-28-0 | ##### | NA 130 | Herbicide | Triazine |
| Chlorazine | 580-48-3 | 80806 | NA 777 | N/A | Triazine |
| Cyanatryn | 21689-84-9 | NA 230 | NA 229 | Herbicide | Triazine |
| Cyanazine | 21725-46-2 | ##### | 1640 | Herbicide | Triazine |
| Cyanazine acid | NA 1328 | NA 178 | NA 178 | Breakdown | Triazine |
| | | | | product | |
| Cyanazine amide | NA 1170 | NA 176 | NA 164 | Breakdown | Triazine |
| | | | | product | |
| Cyanazine, other related | NA 1133 | NA 166 | 91640 | Herbicide | Triazine |
| Cyanuric acid | 108-80-5 | 81402 | 2138 | Microbiocide, | Triazine |
| | | | | Water | |
| | | | | Treatment | |
| Cyanuric acid, monosodium | 2624-17-1 | NA 122 | 1031 | Microbiocide, | Triazine |
| salt | | | | Water | |
| | | | | Treatment | |
| Cybutryne | 28159-98-0 | ##### | 4033 | Microbiocide | Triazine |
| Cyprazine | 22936-86-3 | ##### | 1701 | Herbicide | Triazine |
| Cyromazine | 66215-27-8 | ##### | 2286 | Insecticide | Triazine |
| Deethyl ametryne | NA 1168 | NA 176 | NA 164 | Breakdown | Triazine |
| | | | | product | |
| Deethyl hydroxyatrazine | NA 1330 | NA 179 | NA 178 | Breakdown | Triazine |
| | | | | product | |
| Deethyl-atrazine | NA 1058 | NA 157 | 4051 | Breakdown | Triazine |
| | | | | product | |
| Deethyl-simazine | NA 1047 | NA 154 | 4096 | Breakdown | Triazine |
| | | | | product | |
| Deethylhydroxysimazine | NA 1064 | NA 159 | 5031 | Breakdown | Triazine |
| | | | | product | |
| Deisopropyl atrazine | NA 1169 | NA 176 | NA 164 | Breakdown | Triazine |
| | | | | product | |
| Deisopropyl hydroxyatrazine | NA 1331 | NA 179 | NA 178 | Breakdown | Triazine |
| | | | | product | |
| Deisopropyl prometryn | NA 1334 | NA 179 | NA 179 | Breakdown | Triazine |
| | | | | product | |
| Desamino-metribuzin | NA 1473 | NA 219 | NA 219 | Breakdown | Triazine |
| | | | | product | |
| Desmetryne | 1014-69-3 | 80810 | NA 779 | Herbicide | Triazine |
| Diaminochlorotriazine | NA 856 | NA 159 | 5028 | Breakdown | Triazine |
| | | | | product | |
| Diaminohydroxytriazine | NA 857 | NA 159 | 5032 | Breakdown | Triazine |
| | | | | product | |
| Dichloro-s-triazinetrione | 2782-57-2 | 81401 | 204 | Microbiocide, | Triazine |
| | | | | Water | |
| | | | | Treatment | |
| Dichloroisocyanuric acid, | 2244-21-5 | 81403 | 2 | Microbiocide, | Triazine |
| potassium salt | | | | Water | |
| | | | | Treatment | |
| Didealkyl atrazine | NA 1329 | NA 178 | NA 178 | Breakdown | Triazine |
| | | | | product | |
| Didealkyl hydroxyatrazine | NA 1332 | NA 179 | NA 178 | Breakdown | Triazine |
| | | | | product | |
| Diketo-desamino-metribuzin | NA 1475 | NA 219 | NA 219 | Breakdown | Triazine |
| | | | | product | |
| Diketo-metribuzin | NA 1474 | NA 219 | NA 219 | Breakdown | Triazine |
| | | | | product | |
| Dimethametryn | 22936-75-0 | 80815 | 2287 | Herbicide | Triazine |
| Dipropetryn | 4147-51-7 | ##### | 2532 | Herbicide | Triazine |
| Eglinazine | 6616-80-4 | NA 207 | NA 207 | Herbicide | Triazine |
| Eglinazine-ethyl | 6616-80-4 | NA 218 | NA 217 | Herbicide | Triazine |
| Ethiozin (ebuzin/tycor) | 64529-56-2 | ##### | NA 15 | N/A | Triazine |
| Hexahydro-1,3,5-triethyl-S- | 108-74-7 | NA 125 | 1670 | Herbicide | Triazine |
| triazine | | | | | |
| Hexahydro-1,3,5-tris (2- | 1028251 | 83301 | 1171 | Microbiocide | Triazine |
| hydroxyethyl)-s-triazine | | | | | |
| Hexahydro-1,3,5-tris(2- | 25254-50-6 | ##### | 1915 | N/A | Triazine |
| hydroxypropyl)-s-triazine | | | | | |
| Hexahydro-1,3,5-tris(3- | 751061 | ##### | NA 127 | N/A | Triazine |
| methoxypropyl)-1,3,5-triazine | | | | | |
| Hexazinone | 51235-04-2 | ##### | 1871 | Herbicide | Triazine |
| Hydroxyatrazine | 2163-68-0 | NA 178 | NA 178 | Breakdown | Triazine |
| | | | | product | |
| Ipazin | 1912-25-0 | ##### | NA 130 | N/A | Triazine |
| Isomethiozin | 57052-04-7 | ##### | NA 875 | N/A | Triazine |
| Metamitron | 41394-05-2 | NA 135 | 2672 | Herbicide | Triazine |
| Methometon | 1771-07-9 | ##### | NA 156 | N/A | Triazine |
| Methoprotryne | 841-06-5 | ##### | NA 154 | Herbicide | Triazine |
| Metribuzin | 21087-64-9 | ##### | 1692 | Herbicide | Triazine |
| Metribuzin-DA | NA 1053 | NA 156 | 4079 | Breakdown | Triazine |
| | | | | product | |
| Mono (trichloro) tetra | 30622-37-8 | 81406 | 856 | Microbiocide, | Triazine |
| (monopotassium dichloro) | | | | Water | |
| penta-s-triazine | | | | Treatment | |
| MPMT | 845-52-3 | 82701 | 2695 | N/A | Triazine |
| Potassium 1,3-dichloro-2,4,6- | NA 173 | NA 346 | 1650 | N/A | Triazine |
| trioxo hexahydro-s-triazine | | | | | |
| Prefox (cyprazine with | 8071-40-7 | ##### | NA 886 | Herbicide | Triazine |
| ethiolate) | | | | | |
| Procyanazine | 32889-48-8 | ##### | NA 908 | Herbicide | Triazine |
| Proglinazine | 68228-20-6 | NA 209 | NA 209 | Herbicide | Triazine |
| Proglinazine-ethyl | 68228-18-2 | NA 218 | NA 218 | Herbicide | Triazine |
| Prometon | 1610-18-0 | 80804 | 499 | Herbicide | Triazine |
| Prometryn | 7287-19-6 | 80805 | 502 | Herbicide | Triazine |
| Propazine | 139-40-2 | 80808 | 504 | Herbicide | Triazine |
| Pymetrozine | 123312-89-0 | ##### | 5232 | Insecticide | Triazine |
| Sebuthylazine | 7286-69-3 | ##### | NA 127 | N/A | Triazine |
| Secbumeton | 26259-45-0 | ##### | 2849 | Herbicide | Triazine |
| Simazine | 122-34-9 | 80807 | 531 | Herbicide | Triazine |
| Simetone | 673-04-1 | ##### | 3532 | Herbicide | Triazine |
| Simetryn | 1014-70-6 | ##### | 4097 | N/A | Triazine |
| Sodium dichloro-s- | 2893-78-9 | 81404 | 205 | Microbiocide, | Triazine |
| triazinetrione | | | | Water | |
| | | | | Treatment | |
| Sodium dichloro-s- | 51580-86-0 | 81407 | 1818 | Microbiocide, | Triazine |
| triazinetrione dihydrate | | | | Water | |
| | | | | Treatment | |
| Sym-triazine | 108-80-5 | 81402 | 893 | Microbiocide, | Triazine |
| | | | | Water | |
| | | | | Treatment | |
| Terbumeton | 33693-04-8 | NA 190 | NA 189 | Herbicide | Triazine |
| Terbuthylazine | 5915-41-3 | 80814 | 3004 | Algaecide, | Triazine |
| | | | | Herbicide, | |
| | | | | Microbiocide | |
| Terbutryn | 886-50-0 | 80813 | 1691 | Herbicide | Triazine |
| Terbutryn, other related | NA 1136 | NA 167 | 91691 | Herbicide | Triazine |
| Triaziflam | 131475-57-5 | NA 234 | NA 233 | Herbicide | Triazine |
| Trichloro melamine | 2107336 | 77101 | 1023 | Microbiocide | Triazine |
| Trichloro-s-triazinetrione | 87-90-1 | 81405 | 407 | Microbiocide, | Triazine |
| | | | | Water | |
| | | | | Treatment | |
| Trietazine | 1912-26-1 | 80809 | NA 778 | Herbicide | Triazine |
| 1,3-Diphenyl urea | 102-07-8 | NA 186 | NA 185 | Plant Growth | Urea |
| | | | | Regulator | |
| 3,4-Dichloromethylphenyl urea | NA 1335 | NA 179 | NA 179 | Breakdown | Urea |
| | | | | product | |
| 3,4-Dichlorophenyl urea | 154536 | NA 179 | NA 179 | Breakdown | Urea |
| | | | | product | |
| 3-(Trifluoromethyl)phenyl urea | 13114-87-9 | NA 179 | NA 178 | Breakdown | Urea |
| | | | | product | |
| Amidosulfuron | 120923-37-7 | NA 183 | NA 183 | Herbicide | Sulfonylurea |
| Anisuron | 2689-43-2 | ##### | NA 138 | Herbicide | Benzoylurea |
| Azimsulfuron | 120162-55-2 | ##### | NA 112 | Herbicide | Sulfonylurea |
| Bensulfuron | 99283-01-9 | NA 181 | NA 180 | Herbicide | Sulfonylurea |
| Bensulfuron methyl | 83055-99-6 | ##### | 2263 | Herbicide | Sulfonylurea |
| Bentaluron | NA 1405 | NA 197 | NA 197 | Fungicide | Urea |
| Benzthiazuron | 1929-88-0 | ##### | NA 133 | Herbicide | Urea |
| Buthiuron | 30043-55-1 | NA 230 | NA 230 | Herbicide | Urea |
| Buturon | 3766-60-7 | ##### | NA 119 | Herbicide | Urea |
| Chlorbromuron | 13360-45-7 | 90701 | 1733 | Herbicide | Urea |
| Chloreturon | 20782-58-5 | NA 231 | NA 230 | Herbicide | Phenylurea |
| Chlorimuron | 99283-00-8 | NA 204 | NA 203 | Herbicide | Sulfonylurea |
| Chlorimuron ethyl | 90982-32-4 | ##### | 2458 | Herbicide | Sulfonylurea |
| Chlorotoluron | 15545-48-9 | ##### | NA 122 | Herbicide | Urea |
| Chloroxuron | 1982-47-4 | 25501 | 576 | Herbicide | Urea |
| Chlorsulfuron | 64902-72-3 | ##### | 2143 | Herbicide | Sulfonylurea |
| Cinosulfuron | 94593-91-6 | NA 183 | NA 183 | Herbicide | Sulfonylurea |
| Cumyluron | 99485-76-4 | NA 228 | NA 228 | Herbicide | Urea |
| Cyclosulfamuron | 136849-15-5 | NA 204 | NA 203 | Herbicide | Sulfonylurea |
| Cycluron | 2163-69-1 | 35504 | NA 306 | Herbicide | Urea |
| Daimuron | 42609-52-9 | NA 204 | NA 204 | Herbicide | Phenylurea |
| Demethylfluometuron | NA 1333 | NA 179 | NA 178 | Breakdown | Urea |
| | | | | product | |
| Dichloral urea | 116-52-9 | ##### | NA 115 | Herbicide | Urea |
| Difenoxuron | 14214-32-5 | ##### | NA 156 | Herbicide | Urea |
| Diflubenzuron | 35367-38-5 | ##### | 1992 | Insecticide | Benzoylurea |
| Dimefuron | 34205-21-5 | NA 186 | NA 185 | Herbicide | Urea |
| Diuron | 330-54-1 | 35505 | 231 | Herbicide | Urea |
| Ethametsulfuron | 111353-84-5 | NA 212 | NA 212 | Herbicide | Sulfonylurea |
| Ethametsulfuron-methyl | 97780-06-8 | ##### | NA 43 | Herbicide | Sulfonylurea |
| Ethidimuron | 30043-49-3 | ##### | NA 994 | Herbicide | Urea |
| Ethoxysulfuron | 126801-58-9 | NA 226 | NA 225 | Herbicide | Sulfonylurea |
| Fenuron | 101-42-8 | 35507 | 4072 | Herbicide | Urea |
| Fenuron trichloroacetate | 4482-55-7 | 35508 | 5035 | Herbicide | Urea |
| Flazasulfuron | 104040-78-0 | NA 229 | NA 228 | Herbicide | Sulfonylurea |
| Flucycloxuron | 113036-88-7 | ##### | NA 109 | Herbicide | Benzoylurea |
| Flucycloxuron, (E) | 94050-52-9 | NA 201 | NA 200 | Herbicide | Benzoylurea |
| Flufenoxuron | 101463-69-8 | ##### | NA 901 | Insecticide | Benzoylurea |
| Fluometuron | 2164-17-2 | 35503 | 166 | Herbicide | Urea |
| Fluothiuron | 33439-45-1 | NA 232 | NA 232 | Herbicide | Phenylurea |
| Flupyrsulfuron | 150315-10-9 | NA 205 | NA 204 | Herbicide | Sulfonylurea |
| Flupyrsulfuron-methyl | NA 1460 | NA 216 | NA 216 | Herbicide | Sulfonylurea |
| Flupyrsulfuron-methyl, sodium | 144740-54-5 | NA 218 | NA 217 | Herbicide | Sulfonylurea |
| salt | | | | | |
| Forchlorfenuron | 68157-60-8 | ##### | 5557 | Plant Growth | Urea |
| | | | | Regulator | |
| Halosulfuron | 100784-20-1 | ##### | 3919 | Herbicide | Sulfonylurea |
| Hexaflumuron | 86479-06-3 | ##### | 3899 | Insecticide | Benzoylurea |
| Imazosulfuron | 122548-33-8 | NA 229 | NA 229 | Herbicide | Sulfonylurea |
| Iodosulfuron methyl | 185119-76-0 | NA 212 | NA 211 | Herbicide | Sulfonylurea |
| Iodosulfuron methyl, sodium | 144550-36-7 | NA 212 | NA 211 | Herbicide | Sulfonylurea |
| salt | | | | | |
| Isononuron | 28805-78-9 | ##### | NA 151 | Herbicide | Urea |
| Isoproturon | 34123-59-6 | ##### | NA 155 | Herbicide | Urea |
| Isouron | 55861-78-4 | ##### | NA 101 | Herbicide | Urea |
| Linuron | 330-55-2 | 35506 | 361 | Herbicide | Urea |
| Lufenuron | 103055-07-8 | NA 184 | NA 183 | Insecticide | Benzoylurea |
| Mesosulfuron-methyl | 208465-21-8 | ##### | NA 993 | Herbicide | Sulfonylurea |
| Methabenzthiazuron | 18691-97-9 | ##### | NA 133 | Herbicide | Urea |
| Methiuron | 21540-35-2 | ##### | NA 145 | Herbicide | Urea |
| Methyldymron | 42609-73-4 | NA 205 | NA 204 | Herbicide | Phenylurea |
| Metobenzuron | 111578-32-6 | ##### | NA 37 | Herbicide | Urea |
| Metobromuron | 3060-89-7 | 35901 | NA 313 | Herbicide | Urea |
| Metoxuron | 19937-59-8 | ##### | NA 135 | Herbicide | Urea |
| Metsulfuron | 5585-64-8 | NA 183 | NA 182 | Herbicide | Sulfonylurea |
| Metsulfuron-methyl | 74223-64-6 | ##### | 2222 | Herbicide | Sulfonylurea |
| Monisouron | 55807-46-0 | NA 233 | NA 232 | Herbicide | Urea |
| Monolinuron | 1746-81-2 | ##### | NA 119 | Herbicide | Urea |
| Monolinuron with paraquat | 69312-67-0 | ##### | NA 119 | Herbicide | Urea, Bipyridylium |
| dichloride | | | | | |
| Monuron | 150-68-5 | 35501 | 408 | Herbicide | Urea |
| Monuron-TCA | 140-41-0 | 35502 | 409 | Herbicide | Urea |
| Neburon | 555-37-3 | 12001 | 424 | Herbicide | Urea |
| Nicosulfuron | 111991-09-4 | ##### | 3829 | Herbicide | Sulfonylurea |
| Norea | 18530-56-8 | 35801 | 435 | Herbicide | Urea |
| Norea (stereochemistry | 2163-79-3 | NA 201 | NA 200 | Herbicide | Urea |
| unspecified) | | | | | |
| Norea, other related | NA 1096 | NA 163 | 90435 | Herbicide | Urea |
| Novaluron | 116714-46-6 | NA 226 | NA 226 | Herbicide | Benzoylurea |
| Oxasulfuron | 144651-06-9 | ##### | NA 991 | Herbicide | Sulfonylurea |
| Parafluron | 7159-99-1 | 35510 | NA 307 | Herbicide | Urea |
| Pencycuron | 66063-05-6 | ##### | NA 103 | Fungicide | Urea |
| Penfluron | 35367-31-8 | ##### | NA 100 | Herbicide | Urea |
| Phenobenzuron | 449583 | ##### | NA 133 | Herbicide | Benzoylurea |
| Primisulfuron-methyl | 86209-51-0 | ##### | 5103 | Herbicide | Sulfonylurea |
| Prosulfuron | 94125-34-5 | ##### | 5115 | Herbicide | Sulfonylurea |
| Pyrazosulfuron | 98389-04-9 | NA 205 | NA 205 | Herbicide | Sulfonylurea |
| Pyrazosulfuron-ethyl | 93697-74-6 | NA 218 | NA 217 | Herbicide | Sulfonylurea |
| Rimsulfuron | 122931-48-0 | ##### | 3835 | Herbicide | Sulfonylurea |
| Siduron | 1982-49-6 | 35509 | 603 | Herbicide | Urea |
| Sulfometuron | 74223-56-6 | NA 206 | NA 205 | Herbicide | Sulfonylurea |
| Sulfometuron methyl | 74222-97-2 | ##### | 2149 | Herbicide | Sulfonylurea |
| Sulfosulfuron | 141776-32-1 | 85601 | 5136 | Herbicide | Sulfonylurea |
| Tebuthiuron | 34014-18-1 | ##### | 1810 | Herbicide | Urea |
| Teflubenzuron | 83121-18-0 | ##### | NA 33 | Insecticide | Benzoylurea |
| Tetrafluoron | 27954-37-6 | ##### | NA 145 | Herbicide | Phenylurea |
| Thiazafluron | 25366-23-8 | NA 190 | NA 189 | Herbicide | Urea |
| Thidiazuron | 51707-55-2 | ##### | 2162 | Defoliant, Plant | Urea |
| | | | | Growth | |
| | | | | Regulator | |
| Thifensulfuron | 79277-67-1 | NA 183 | NA 182 | Herbicide | Sulfonylurea |
| Thifensulfuron-methyl | 79277-27-3 | ##### | 2237 | Herbicide | Sulfonylurea |
| Triasulfuron | 82097-50-5 | ##### | 5100 | Herbicide | Sulfonylurea |
| Tribenuron | 106040-48-6 | NA 206 | NA 205 | Herbicide | Sulfonylurea |
| Tribenuron methyl | 101200-48-0 | ##### | 2338 | Herbicide | Sulfonylurea |
| Triflumuron | 64628-44-0 | ##### | 2961 | Insecticide | Benzoylurea |
| Triflusulfuron-methyl | 126535-15-7 | ##### | 3875 | Herbicide | Sulfonylurea |
| Trimeturon | 3050-27-9 | ##### | NA 121 | Herbicide | Urea |
| Tritosulfuron | 142469-14-5 | NA 234 | NA 234 | Herbicide | Sulfonylurea |
| Bromacil | 314-40-9 | 12301 | 83 | Herbicide | Uracil |
| Bromacil, dimethylamine salt | 69484-13-5 | 12304 | 840 | Herbicide | Uracil |
| Bromacil, lithium salt | 53404-19-6 | 12302 | 1573 | Herbicide | Uracil |
| Bromacil, sodium salt | 69484-12-4 | 12303 | 842 | Herbicide | Uracil |
| Butafenacil | 134605-64-4 | ##### | NA 992 | Herbicide | Uracil |
| Flupropacil | 120890-70-2 | NA 229 | NA 229 | Herbicide | Uracil |
| Isocil | 314-42-1 | 11801 | NA 119 | Herbicide | Uracil |
| Lenacil | 95177 | ##### | NA 155 | Herbicide | Uracil |
| Terbacil | 5902-51-2 | 12701 | 532 | Herbicide | Uracil |
| 2-Hydroxy alachlor | NA 943 | NA 183 | 2602 | Breakdown | Chloroacetanilide |
| | | | | product | |
| 3,4,5-tribromo salicylanilide, | NA 1122 | NA 165 | 90833 | Microbiocide | Anilide |
| other related | | | | | |
| 3,5-dibromosalicylanilide | 2577-72-2 | 77405 | 1256 | Microbiocide | Anilide |
| 5,4-dibromosalicylanilide | NA 1024 | NA 122 | 1071 | Microbiocide | Anilide |
| Aatram (propachlor with | 8070-76-6 | 80816 | NA 781 | Herbicide | Triazine, |
| atrazine) (019101 + 080803) | | | | | Chloroacetanilide |
| Acetochlor | 34256-82-1 | ##### | 2349 | Herbicide | Chloroacetanilide |
| Alachlor | 15972-60-8 | 90501 | 678 | Herbicide | Chloroacetanilide |
| Benodanil | 15310-01-7 | ##### | NA 154 | Fungicide | Anilide |
| Butachlor | 23184-66-9 | ##### | 4056 | Herbicide | Chloroacetanilide |
| Butenachlor | 87310-56-3 | NA 206 | NA 206 | Herbicide | Chloroacetanilide |
| Clomeprop | 84496-56-0 | NA 204 | NA 203 | Herbicide | Anilide |
| Cyprofuram | 69581-33-5 | NA 186 | NA 185 | Fungicide | Anilide |
| Cypromid | 2759-71-9 | 26101 | 178 | Herbicide | Anilide |
| Delachlor | 24353-58-0 | ##### | NA 132 | Herbicide | Chloroacetanilide |
| Diethatyl-ethyl | 38727-55-8 | ##### | 1995 | Herbicide | Chloroacetanilide |
| Diflufenican | 83164-33-4 | NA 184 | NA 183 | Herbicide | Anilide |
| Dimethachlor | 50563-36-5 | NA 184 | NA 183 | Herbicide | Chloroacetanilide |
| Etobenzanid | 79540-50-4 | NA 229 | NA 228 | Herbicide | Anilide |
| Fenasulam | 78357-48-9 | NA 231 | NA 231 | Herbicide | Anilide |
| Flufenacet | 142459-58-3 | ##### | 5293 | Herbicide | Anilide |
| Flufenican | NA 1568 | NA 234 | NA 234 | Herbicide | Anilide |
| Flutolanil | 66332-96-5 | ##### | 2305 | Fungicide | Anilide |
| Mefenacet | 73250-68-7 | NA 189 | NA 188 | Herbicide | Anilide |
| Mefluidide | 53780-34-0 | ##### | 5082 | Herbicide, | Anilide |
| | | | | Plant Growth | |
| | | | | Regulator | |
| Mefluidide, diethanolamine salt | 53780-36-2 | ##### | 1955 | Herbicide | Anilide |
| Mefluidide, potassium salt | 83601-83-6 | ##### | 5083 | Herbicide | Anilide |
| Mepronil | 55814-41-0 | NA 189 | NA 188 | Fungicide | Anilide |
| Metazachlor | 67129-08-2 | NA 184 | NA 183 | Herbicide | Chloroacetanilide |
| Metolachlor | 51218-45-2 | ##### | 1996 | Herbicide | Chloroacetanilide |
| Metolachlor, (S) | 87392-12-9 | ##### | 5133 | Herbicide | Chloroacetanilide |
| Monalide | 7287-36-7 | ##### | NA 134 | Herbicide | Anilide |
| Naproanilide | 52570-16-8 | NA 233 | NA 232 | Herbicide | Anilide |
| Ofurace | 58810-48-3 | NA 189 | NA 188 | Fungicide | Anilide |
| Oxadixyl | 77732-09-3 | ##### | 3539 | Fungicide | Anilide |
| Perfluidone | 37924-13-3 | ##### | 1895 | Herbicide | Anilide |
| Perfluidone, diethanolamine | NA 991 | NA 671 | 1896 | Herbicide | Anilide |
| salt | | | | | |
| Pretilachlor | 51218-49-6 | NA 184 | NA 184 | Herbicide | Chloroacetanilide |
| Propachlor | 1918-16-7 | 19101 | 511 | Herbicide | Chloroacetanilide |
| Propanil | 709-98-8 | 28201 | 503 | Herbicide | Anilide |
| Propisochlor | 86763-47-5 | NA 233 | NA 233 | Herbicide | Chloroacetanilide |
| Prynachlor | 21267-72-1 | ##### | NA 138 | Herbicide, | Chloroacetanilide |
| | | | | Plant Growth | |
| | | | | Regulator | |
| Pyracarbolid | 24691-76-7 | NA 210 | NA 209 | Fungicide | Anilide |
| Salicylanilide | 87-17-2 | 77407 | 450 | Fungicide, | Anilide |
| | | | | Microbiocide | |
| Sodium salicylanilide | 251930 | ##### | NA 145 | Fungicide | Anilide |
| Thenylchlor | 96491-05-3 | NA 229 | NA 229 | Herbicide | Chloroacetanilide |
| Triclocarban | 101-20-2 | 27901 | 844 | N/A | Anilide |
| 1,2,4-triazole | 288-88-0 | NA 968 | 3560 | N/A | Azole |
| 1H-benzo triazole | 95-14-7 | NA 493 | 2418 | N/A | Azole |
| 2,2′-Dithiobisbenzothiazole | 120-78-5 | 9202 | NA 109 | Fungicide, | Mercaptobenzothiazole |
| | | | | Microbiocide, | |
| | | | | Insecticide | |
| 2-(2′-hydroxy-5-methyl | 2440-22-4 | NA 103 | 2607 | Fungicide | Azole |
| phenyl)-benzotriazole | | | | | |
| 2-(dithiocyanomethylthio)- | NA 1346 | NA 192 | NA 191 | Fungicide | Mercaptobenzothiazole |
| benzothiazole | | | | | |
| 2- | 79910-32-0 | ##### | NA 925 | Microbiocide | Mercaptobenzothiazole |
| (thiocyanomethylthio)benzothiazole | | | | | |
| with 2′-hydroxyethyl-2,3- | | | | | |
| dibromopropionate | | | | | |
| 2-EEEBC | 62732-91-6 | ##### | 1983 | Fungicide | Benzimidazole |
| 2-Mercaptobenzothiazole | 149-30-4 | 51701 | 556 | Fungicide, | Mercaptobenzothiazole |
| | | | | Microbiocide | |
| 2-Mercaptobenzothiazole, 1- | 5468-43-9 | 51702 | NA 423 | Fungicide, | Mercaptobenzothiazole |
| (hydroxyethyl)pyridinium salt | | | | Microbiocide | |
| 2-Mercaptobenzothiazole, | 53404-53-8 | 51708 | NA 426 | Fungicide, | Mercaptobenzothiazole |
| ferric salt | | | | Microbiocide | |
| 2-Mercaptobenzothiazole, | 53404-58-3 | 9201 | NA 108 | Fungicide, | Mercaptobenzothiazole |
| manganese salt | | | | Microbiocide, | |
| | | | | Insecticide | |
| 2-Mercaptobenzothiazole, | 5902-85-2 | 51706 | NA 424 | Fungicide, | Mercaptobenzothiazole |
| monoethanolammonium salt | | | | Microbiocide | |
| 2-Mercaptobenzothiazole, | 7778-70-3 | 51707 | NA 425 | Fungicide, | Mercaptobenzothiazole |
| potassium salt | | | | Microbiocide | |
| 2-Mercaptobenzothiazole, | 2492-26-4 | 51704 | 3064 | Microbiocide, | Mercaptobenzothiazole |
| sodium salt | | | | Fungicide | |
| 2-Mercaptobenzothiazole, | 2492-26-4 | 51704 | 613 | Fungicide, | Mercaptobenzothiazole |
| sodium salt | | | | Microbiocide | |
| 2-Mercaptobenzothiazole, zinc | 155-04-4 | 51705 | 1449 | Fungicide, | Mercaptobenzothiazole, |
| salt | | | | Microbiocide | Inorganic-Zinc |
| 5-Chloro-2- | 5331-91-9 | NA 200 | NA 199 | N/A | Mercaptobenzothiazole |
| mercaptobenzothiazole | | | | | |
| 5-Chloro-2- | 5406-97-3 | 51703 | 1238 | Microbiocide | Mercaptobenzothiazole |
| mercaptobenzothiazole, lauryl | | | | | |
| pyridinium salt | | | | | |
| 5-Chloro-2- | 5406-97-3 | NA 421 | 1597 | Microbiocide, | Mercaptobenzothiazole |
| mercaptobenzothiazole, lauryl | | | | Fungicide | |
| pyridinium salt | | | | | |
| 5-Chloro-2- | 53404-93-6 | 51709 | NA 427 | Microbiocide, | Mercaptobenzothiazole, |
| mercaptobenzothiazole, zinc | | | | Fungicide | Inorganic-Zinc |
| salt | | | | | |
| Azaconazole | 60207-31-0 | ##### | NA 105 | Insecticide, | Azole |
| | | | | Fungicide | |
| Benomyl | 17804-35-2 | 99101 | 1552 | Fungicide | Benzimidazole |
| Bitertanol | 55179-31-2 | ##### | NA 959 | Fungicide | Azole |
| Bromuconazole | 116255-48-2 | ##### | NA 8 | Fungicide | Azole |
| Carbendazim | 10605-21-7 | ##### | 2176 | Fungicide | Benzimidazole |
| Carbendazim hydrochloride | 37574-18-8 | ##### | NA 121 | Fungicide | Benzimidazole |
| Carbendazim phosphate | 52316-55-9 | 99102 | 1974 | Fungicide | Benzimidazole |
| CGA-64251 | 60207-93-4 | ##### | 2452 | Fungicide | Azole |
| Chlorfenapyr | 122453-73-0 | ##### | 3938 | Insecticide | Pyrazole |
| Chlorflurazole | 3615-21-2 | ##### | NA 139 | N/A | Benzimidazole |
| Cyazofamid | 120116-88-3 | NA 216 | NA 215 | Fungicide | Azole |
| Cypendazole | 28559-00-4 | ##### | NA 156 | N/A | Benzimidazole |
| Cyproconazole | 94361-06-5 | ##### | 5105 | Fungicide | Azole |
| Difenoconazole | 119446-68-3 | ##### | 5024 | Fungicide | Azole |
| Diniconazole | 83657-18-5 | ##### | 2500 | Fungicide | Azole |
| Diniconazole (stereochemistry | 83657-24-3 | NA 200 | NA 200 | Fungicide | Azole |
| unspecified) | | | | | |
| Fenapanil | 61019-78-1 | ##### | 2083 | Fungicide | Azole |
| Fenbuconazole | 114369-43-6 | ##### | 3905 | Fungicide | Azole |
| Fluotrimazole | 31251-03-3 | NA 207 | NA 207 | Fungicide | Azole |
| Fluquinconazole | 136426-54-5 | NA 184 | NA 183 | Fungicide | Azole |
| Flusilazole | 85509-19-9 | ##### | 2278 | Fungicide | Azole |
| Flutriafol | 76674-21-0 | ##### | NA 107 | Fungicide | Azole |
| Fuberidazole | 3878-19-1 | ##### | NA 150 | Fungicide | Benzimidazole |
| Furconazole | 112839-33-5 | NA 196 | NA 196 | Fungicide | Azole |
| Furconazole-cis | 112839-32-4 | NA 208 | NA 207 | Fungicide | Azole |
| Hexaconazole | 79983-71-4 | ##### | NA 23 | Fungicide | Azole |
| Imazalil | 35554-44-0 | ##### | 2084 | Fungicide | Azole |
| Imazalil sulfate | 58594-72-2 | ##### | NA 921 | Fungicide | Azole |
| Imibenconazole | 86598-92-7 | NA 205 | NA 204 | Fungicide | Azole |
| Isoxachlortole | 141112-06-3 | NA 232 | NA 232 | Herbicide | Cyclopropylisoxazole |
| Metconazole | 125116-23-6 | ##### | NA 101 | Fungicide | Azole |
| Metronidazole | 443-48-1 | ##### | 2208 | Microbiocide | Azole |
| Myclobutanil | 88671-89-0 | ##### | 2245 | Fungicide | Azole |
| Naphthyl triazole stilbene | NA 742 | NA 135 | 2698 | N/A | Azole |
| monosulfonate | | | | | |
| Paclobutrazol | 76738-62-0 | ##### | 2259 | Plant Growth | Azole |
| | | | | Regulator | |
| Penconazole | 66246-88-6 | ##### | NA 109 | Fungicide | Azole |
| Prochloraz | 67747-09-5 | ##### | NA 29 | Fungicide | Azole |
| Prochloraz - manganese | 75747-77-2 | ##### | NA 104 | Fungicide | Azole |
| complex (4:1) | | | | | |
| Prochloraz zinc complex | NA 1555 | NA 226 | NA 226 | Fungicide | Azole |
| Propiconazole | 60207-90-1 | ##### | 2276 | Fungicide | Azole |
| Pyrazoxyfen | 71561-11-0 | NA 189 | NA 189 | Herbicide | Pyrazole |
| R 23 979 imazalil base | NA 451 | NA 809 | 2844 | Fungicide | Azole |
| TCMTB | 21564-17-0 | 35603 | 971 | Microbiocide, | Mercaptobenzothiazole |
| | | | | Fungicide | |
| Tebuconazole | 107534-96-3 | ##### | 3850 | Fungicide | Azole |
| Tebufenpyrad | 119168-77-3 | 90102 | NA 845 | Insecticide | Pyrazole |
| Terrazole | 2593-15-9 | 84701 | 580 | Fungicide | Azole |
| Tetraconazole | 112281-77-3 | ##### | NA 979 | Fungicide | Azole |
| Thiabendazole | 148-79-8 | 60101 | 587 | Fungicide | Benzimidazole |
| Thiabendazole, hypophosphite | 28558-32-9 | 60102 | 1952 | Fungicide | Benzimidazole |
| salt | | | | | |
| Thiophanate | 23564-06-9 | ##### | 1684 | Fungicide | Benzimidazole |
| Thiophanate-methyl | 23564-05-8 | ##### | 1696 | Fungicide | Benzimidazole |
| | | | | | precursor |
| Tolyl triazole | 29395-43-1 | NA 962 | 2950 | N/A | Azole |
| Triadimefon | 43121-43-3 | ##### | 2133 | Fungicide | Azole |
| Triadimenol | 55219-65-3 | ##### | 2307 | Fungicide, | Azole |
| | | | | Breakdown | |
| | | | | product | |
| Triazbutil | 16227-10-4 | ##### | NA 931 | Fungicide | Azole |
| Tricyclazole | 41814-78-2 | ##### | 5002 | Fungicide | Azole |
| Triflumizole | 68694-11-1 | ##### | 2260 | Fungicide | Azole |
| Triflumizole | 99387-89-0 | NA 201 | NA 201 | Fungicide | Azole |
| Triticonazole | 131983-72-7 | ##### | NA 101 | Fungicide | Azole |
| Uniconazole | 83657-22-1 | ##### | NA 38 | Plant Growth | Azole |
| | | | | Regulator | |
| 2-EEEBC | 62732-91-6 | ##### | 1983 | Fungicide | Benzimidazole |
| Benomyl | 17804-35-2 | 99101 | 1552 | Fungicide | Benzimidazole |
| Carbendazim | 10605-21-7 | ##### | 2176 | Fungicide | Benzimidazole |
| Carbendazim hydrochloride | 37574-18-8 | ##### | NA 121 | Fungicide | Benzimidazole |
| Carbendazim phosphate | 52316-55-9 | 99102 | 1974 | Fungicide | Benzimidazole |
| Chlorflurazole | 3615-21-2 | ##### | NA 139 | N/A | Benzimidazole |
| Cypendazole | 28559-00-4 | ##### | NA 156 | N/A | Benzimidazole |
| Fuberidazole | 3878-19-1 | ##### | NA 150 | Fungicide | Benzimidazole |
| Thiabendazole | 148-79-8 | 60101 | 587 | Fungicide | Benzimidazole |
| Thiabendazole, hypophosphite | 28558-32-9 | 60102 | 1952 | Fungicide | Benzimidazole |
| salt | | | | | |
| Thiophanate | 23564-06-9 | ##### | 1684 | Fungicide | Benzimidazole |
| Thiophanate-methyl | 23564-05-8 | ##### | 1696 | Fungicide | Benzimidazole |
| | | | | | precursor |
| 1-(3- | 51971-67-6 | ##### | NA 110 | N/A | Carboxamide |
| chlorophthalimido)cyclohexane | | | | | |
| carboxamide | | | | | |
| Carboxin | 5234-68-4 | 90201 | 1755 | Fungicide | Carboxamide |
| Cisanilide | 34484-77-0 | ##### | NA 144 | Herbicide | Carboxamide |
| Cyclafuramide | 34849-42-8 | ##### | NA 132 | Fungicide | Carboxamide |
| Fenfuram | 24691-80-3 | NA 188 | NA 187 | Fungicide | Carboxamide |
| Furmecyclox (Xyligen B) | 60568-05-0 | ##### | NA 18 | Fungicide | Carboxamide |
| Methfuroxam | 28730-17-8 | ##### | NA 949 | Fungicide | Carboxamide |
| Metsulfovax | 21452-18-6 | NA 189 | NA 188 | Fungicide | Carboxamide |
| Nitrofurazone (not subject to | 59-87-0 | ##### | NA 128 | N/A | Carboxamide |
| FIFRA: 8709-e pm 24) | | | | | |
| Oxycarboxin | 5259-88-1 | 90202 | 1434 | Fungicide | Carboxamide |
| p-Benzoquinone | 61566-21-0 | ##### | NA 133 | N/A | Carboxamide |
| semicarbazone | | | | | |
| Chlozolinate | 72391-46-9 | NA 183 | NA 182 | Fungicide | Dicarboximide |
| Iprodione | 36734-19-7 | ##### | 2081 | Fungicide | Dicarboximide |
| Myclozolin | 54864-61-8 | NA 209 | NA 208 | Fungicide | Dicarboximide |
| N-(2-Ethylhexyl)-1-isopropyl- | 13358-11-7 | ##### | NA 121 | Synergist | Dicarboximide |
| 4-methyl-4-cyclohexene-1,2- | | | | | |
| dicarboximide | | | | | |
| N-octyl bicycloheptene | 113-48-4 | 57001 | 396 | Synergist | Dicarboximide |
| dicarboximide | | | | | |
| Vinclozolin | 50471-44-8 | ##### | 2129 | Fungicide | Dicarboximide |
| Calcium | 5895-18-1 | 14501 | NA 139 | Microbiocide | Dithiocarbamate |
| ethylenebis(dithiocarbamate) | | | | | |
| Cufraneb | 11096-18-7 | NA 181 | NA 180 | Fungicide | Dithiocarbamate |
| Diammonium | 607313 | 14502 | NA 140 | Microbiocide | Dithiocarbamate |
| ethylenebis(dithiocarbamate) | | | | | |
| Ferbam | 14484-64-1 | 34801 | 288 | Fungicide | Dithiocarbamate |
| Ferric nitroso dimethyl | 83542-83-0 | ##### | NA 149 | Microbiocide | Dithiocarbamate |
| dithiocarbamate | | | | | |
| Mancopper | 53988-93-5 | NA 181 | NA 180 | Fungicide | Dithiocarbamate |
| Mancozeb | 2233100 | 14504 | 211 | Fungicide | Dithiocarbamate, |
| | | | | | Inorganic-Zinc |
| Maneb | 12427-38-2 | 14505 | 369 | Fungicide | Dithiocarbamate |
| Maneb and nickel sulfate | 8005-46-7 | ##### | NA 158 | Fungicide | Dithiocarbamate, |
| hexahydrate (014505 + 050505) | | | | | Inorganic-Nickel |
| Manganous | 15339-36-3 | 34802 | NA 304 | Fungicide | Dithiocarbamate |
| dimethyldithiocarbamate | | | | | |
| Mercuric dimethyl | 15415-64-2 | 34808 | 709 | Microbiocide | Inorganic-Mercury, |
| dithiocarbamate | | | | | Heavy metal, |
| | | | | | Dithiocarbamate |
| Metam sodium, dihydrate | 137-42-8 | 39003 | NA 163 | Fumigant, | Dithiocarbamate |
| | | | | Fungicide, | |
| | | | | Microbiocide, | |
| | | | | Algaecide, | |
| | | | | Nematicide | |
| Metam-sodium | 6734-80-1 | 39003 | 616 | Fumigant, | Dithiocarbamate |
| | | | | Herbicide, | |
| | | | | Fungicide, | |
| | | | | Microbiocide, | |
| | | | | Algaecide, | |
| | | | | Nematicide | |
| Metiram | 9006-42-2 | 14601 | 493 | Fungicide | Dithiocarbamate, |
| | | | | | Inorganic-Zinc |
| Morpholinomethyl | 31848-11-0 | ##### | NA 158 | Microbiocide | Dithiocarbamate |
| dimethyldithiocarbamate | | | | | |
| Nabam | 142-59-6 | 14503 | 417 | Fungicide, | Dithiocarbamate |
| | | | | Herbicide | |
| Phenylmercuric | 32407-99-1 | 66008 | NA 555 | Microbiocide, | Organomercury, |
| dimethyldithiocarbamate | | | | Fungicide | Dithiocarbamate, |
| | | | | | Heavy metal |
| Potassium ammonium | 22221-14-3 | 14507 | NA 141 | Microbiocide | Dithiocarbamate |
| ethylenebis(dithiocarbamate) | | | | | |
| Potassium dimethyl dithiocarbamate | 128-03-0 | 34803 | 1934 | Microbiocide | Dithiocarbamate |
| Potassium N-hydroxymethyl- | 51026-28-9 | ##### | 1824 | N/A | Dithiocarbamate |
| N-methyldithio carbamate | | | | | |
| Potassium N-methyldithio | 137-41-7 | 39002 | 970 | Fumigant, | Dithiocarbamate |
| carbamate | | | | Fungicide, | |
| | | | | Microbiocide, | |
| | | | | Algaecide, | |
| | | | | Nematicide | |
| Propineb | 12071-83-9 | ##### | NA 155 | Fungicide, | Dithiocarbamate, |
| | | | | Microbiocide | Inorganic-Zinc |
| Sodium dimethyl dithio | 128-04-1 | 34804 | 548 | Fungicide | Dithiocarbamate |
| carbamate | | | | | |
| Sulfallate | 95-06-7 | 39001 | 115 | Herbicide | Dithiocarbamate |
| Tert-butyl dimethyl trithio | 3304-97-0 | 34807 | 1383 | Rodent | Dithiocarbamate |
| peroxycarbamate | | | | Repellent | |
| Tert- | NA 1130 | NA 166 | 91383 | N/A | Dithiocarbamate |
| butyldimethyltrithioperoxycarbamate, | | | | | |
| other related | | | | | |
| Thiram | 137-26-8 | 79801 | 589 | Fungicide | Dithiocarbamate |
| Thiram and NAD and IBA and | 75497-92-6 | 56011 | NA 463 | Fungicide, | Dithiocarbamate |
| 2-methyl-1- | | | | Plant Growth | |
| naphthaleneacetamide and 2- | | | | Regulator | |
| methyl-1-naphthaleneacetic | | | | | |
| acid | | | | | |
| Tricopper dichloride | 7076-63-3 | ##### | NA 152 | Microbiocide | Dithiocarbamate, |
| dimethyldithiocarbamate | | | | | Inorganic-Copper |
| Zineb | 12122-67-7 | 14506 | 627 | Fungicide | Dithiocarbamate, |
| | | | | | Inorganic-Zinc |
| Zineb-ethylene thiuram | NA 1465 | NA 217 | NA 216 | Fungicide | Dithiocarbamate, |
| disulfide adduct | | | | | Inorganic-Zinc |
| Ziram | 137-30-4 | 34805 | 629 | Fungicide, | Dithiocarbamate, |
| | | | | Microbiocide, | Inorganic-Zinc |
| | | | | Dog and Cat | |
| | | | | Repellent | |
| Zirm, cyclohexylamine | 16509-79-8 | 34806 | 1328 | Dog and Cat | Dithiocarbamate, |
| complex | | | | Repellent, | Inorganic-Zinc |
| | | | | Fungicide | |
| 2,2′-Dithiobisbenzothiazole | 120-78-5 | 9202 | NA 109 | Fungicide, | Mercaptobenzothiazole |
| | | | | Microbiocide, | |
| | | | | Insecticide | |
| 2-(dithiocyanomethylthio)- | NA 1346 | NA 192 | NA 191 | Fungicide | Mercaptobenzothiazole |
| benzothiazole | | | | | |
| 2- | 79910-32-0 | ##### | NA 925 | Microbiocide | Mercaptobenzothiazole |
| (thiocyanomethylthio)benzothiazole | | | | | |
| with 2′-hydroxyethyl-2,3- | | | | | |
| dibromopropionate | | | | | |
| 2-Mercaptobenzothiazole | 149-30-4 | 51701 | 556 | Fungicide, | Mercaptobenzothiazole |
| | | | | Microbiocide | |
| 2-Mercaptobenzothiazole, 1- | 5468-43-9 | 51702 | NA 423 | Fungicide, | Mercaptobenzothiazole |
| (hydroxyethyl)pyridinium salt | | | | Microbiocide | |
| 2-Mercaptobenzothiazole, | 53404-53-8 | 51708 | NA 426 | Fungicide, | Mercaptobenzothiazole |
| ferric salt | | | | Microbiocide | |
| 2-Mercaptobenzothiazole, | 53404-58-3 | 9201 | NA 108 | Fungicide, | Mercaptobenzothiazole |
| manganese salt | | | | Microbiocide, | |
| | | | | Insecticide | |
| 2-Mercaptobenzothiazole, | 5902-85-2 | 51706 | NA 424 | Fungicide, | Mercaptobenzothiazole |
| monoethanolammonium salt | | | | Microbiocide | |
| 2-Mercaptobenzothiazole, | 7778-70-3 | 51707 | NA 425 | Fungicide, | Mercaptobenzothiazole |
| potassium salt | | | | Microbiocide | |
| 2-Mercaptobenzothiazole, | 2492-26-4 | 51704 | 3064 | Microbiocide, | Mercaptobenzothiazole |
| sodium salt | | | | Fungicide | |
| 2-Mercaptobenzothiazole, | 2492-26-4 | 51704 | 613 | Fungicide, | Mercaptobenzothiazole |
| sodium salt | | | | Microbiocide | |
| 2-Mercaptobenzothiazole, zinc | 155-04-4 | 51705 | 1449 | Fungicide, | Mercaptobenzothiazole, |
| salt | | | | Microbiocide | Inorganic-Zinc |
| 5-Chloro-2- | 5331-91-9 | NA 200 | NA 199 | N/A | Mercaptobenzothiazole |
| mercaptobenzothiazole | | | | | |
| 5-Chloro-2- | 5406-97-3 | 51703 | 1238 | Microbiocide | Mercaptobenzothiazole |
| mercaptobenzothiazole, lauryl | | | | | |
| pyridinium salt | | | | | |
| 5-Chloro-2- | 5406-97-3 | NA 421 | 1597 | Microbiocide, | Mercaptobenzothiazole |
| mercaptobenzothiazole, lauryl | | | | Fungicide | |
| pyridinium salt | | | | | |
| 5-Chloro-2- | 53404-93-6 | 51709 | NA 427 | Microbiocide, | Mercaptobenzothiazole, |
| mercaptobenzothiazole, zinc | | | | Fungicide | Inorganic-Zinc |
| salt | | | | | |
| TCMTB | 21564-17-0 | 35603 | 971 | Microbiocide, | Mercaptobenzothiazole |
| | | | | Fungicide | |
| Ancymidol | 12771-68-5 | ##### | 1744 | Plant Growth | Pyrimidine |
| | | | | Regulator | |
| Bupirimate | 41483-43-6 | ##### | 2430 | Fungicide | Pyrimidine |
| Cloransulam | 159518-97-5 | NA 231 | NA 231 | Herbicide | Triazolopyrimidine |
| Cloransulam-methyl | 147150-35-4 | ##### | NA 112 | Herbicide | Triazolopyrimidine |
| Dimethirimol | 5221-53-4 | ##### | NA 128 | Fungicide | Pyrimidine |
| Ethirimol | 23947-60-6 | ##### | NA 129 | Fungicide | Pyrimidine |
| Fenarimol | 60168-88-9 | ##### | 1980 | Fungicide | Pyrimidine |
| Ferimzone | 89269-64-7 | NA 203 | NA 202 | Fungicide | Pyrimidine |
| Flurprimidol | 56425-91-3 | ##### | 2320 | Plant Growth | Pyrimidine |
| | | | | Regulator | |
| Nuarimol | 63284-71-9 | ##### | NA 126 | Fungicide | Pyrimidine |
| Pyrimethanil | 53112-28-0 | ##### | NA 30 | Fungicide | Pyrimidine |
| Azoxystrobin | 131860-33-8 | ##### | 4037 | Fungicide | Strobin |
| Dimoxystrobin | 149961-52-4 | NA 211 | NA 210 | Fungicide | Strobin |
| Kresoxim-methyl | 143390-89-0 | ##### | 5451 | Fungicide | Strobin |
| Metominostrobin | 133408-50-1 | NA 211 | NA 210 | Fungicide | Strobin |
| Picoxystrobin | 117428-22-5 | NA 211 | NA 210 | Fungicide | Strobin |
| Pyraclostrobin | 175013-18-0 | NA 211 | NA 210 | Fungicide | Strobin |
| Trifloxystrobin | 141517-21-7 | ##### | 5321 | Fungicide | Strobin |
| chloroneb | 2675-77-6 | 27301 | 135 | Fungicide | Substituted Benzene |
| Chlorothalonil | 1897-45-6 | 81901 | 677 | Fungicide | Substituted Benzene |
| Dichlobenil | 1194-65-6 | 27401 | 112 | Herbicide | Substituted benzene |
| Dichloran | 99-30-9 | 31301 | 81 | Fungicide | Substituted Benzene |
| PCNB | 82-68-8 | 56502 | 464 | Fungicide, | Substituted Benzene |
| | | | | Nematicide, | |
| | | | | Algaecide | |
| PCNB, other related | NA 1100 | NA 163 | 90464 | Fungicide, | Substituted Benzene |
| | | | | Algaecide, | |
| | | | | Nematicide | |
| Captafol | 190444 | 81702 | NA 787 | Fungicide | Thiophthalimide |
| Captafol (cis isomer) | 2939-80-2 | 81701 | 292 | Fungicide | Thiophthalimide |
| Captan | 133-06-2 | 81301 | 104 | Fungicide | Thiophthalimide |
| Captan, other related | NA 1074 | NA 161 | 90104 | Fungicide | Thiophthalimide |
| Folpet | 133-07-3 | 81601 | 294 | Fungicide | Thiophthalimide |
| Merpafol cis isomer | 190444 | 81701 | NA 163 | Fungicide | Thiophthalimide |
| Benalaxyl | 71626-11-4 | ##### | NA 102 | Fungicide | Xylylalanine |
| Furalaxyl | 57646-30-7 | NA 188 | NA 188 | Fungicide | Xylylalanine |
| L-Metalaxyl | 69516-34-3 | ##### | NA 929 | Fungicide | Xylylalanine |
| Mefenoxam | 70630-17-0 | ##### | 4011 | Fungicide | Xylylalanine |
| Mefenoxam, other related | NA 1152 | NA 168 | 94011 | Fungicide | Xylylalanine |
| Metalaxyl | 57837-19-1 | ##### | 2132 | Fungicide | Xylylalanine |
| Metalaxyl-M | 70630-17-0 | NA 219 | NA 218 | Fungicide | Xylylalanine |
| 1-(Alkyl* amino)-3- | 61791-67-1 | 67311 | NA 586 | Microbiocide | Alkyl Amino Propane |
| aminopropane *(47% C12, | | | | | |
| 18% C14, 10% C18, 9% C10, | | | | | |
| 8% C16, 8% C8) | | | | | |
| 1-(Alkyl* amino)-3- | 61791-63-7 | 67301 | NA 583 | Microbiocide | Alkyl Amino Propane |
| aminopropane *(as in fatty | | | | | |
| acids of coconut oil) | | | | | |
| 1-(Alkyl* amino)-3- | 68911-78-4 | 67306 | NA 585 | Microbiocide | Alkyl Amino Propane |
| aminopropane diacetate | | | | | |
| *(37% C18′, 29% C16, | | | | | |
| 23% C18, 3% C16′, 3% C14, | | | | | |
| 1.5% C18″, 1% C14′, 1% C12, | | | | | |
| 0.5% C15) | | | | | |
| 1-(Alkyl* amino)-3- | NA 1191 | 67313 | NA 167 | Microbiocide | Alkyl Amino Propane |
| aminopropane diacetate *(as in | | | | | |
| fatty acids of coconut oil) | | | | | |
| 1-(Alkyl* amino)-3- | NA 1192 | 67319 | NA 167 | Microbiocide, | Alkyl Amino Propane |
| aminopropane monoacetate | | | | Algaecide | |
| *(47% C12, 18% C14, 10% C18, | | | | | |
| 9% C10, 8% C16, 8% C8) | | | | | |
| 1-(Alkyl* amino)-3- | NA 1193 | 67304 | NA 167 | Microbiocide | Alkyl Amino Propane |
| aminopropane propionate- | | | | | |
| copper acetate complex | | | | | |
| 1-(alkyl-amino)-3- | NA 1494 | NA 221 | NA 221 | Microbiocide | Alkyl Amino Propane |
| aminopropane hydrochloride | | | | | |
| 1-(alkylamino)-3- | 68155-37-3 | 67310 | 1830 | Microbiocide | Alkyl Amino Propane |
| aminopropane | | | | | |
| 1-(alkylamino)-3- | NA 1495 | NA 221 | NA 221 | Microbiocide | Alkyl Amino Propane |
| carboxymethylaminopropane | | | | | |
| 1-Alkyl (C6 to C18) amino-3- | NA 994 | NA 721 | 2365 | Microbiocide | Alkyl Amino Propane |
| aminopropane acetate | | | | | |
| 1-alkyl (C6-C18) amino-3- | 61791-64-8 | 67302 | 1954 | Microbiocide | Alkyl Amino Propane |
| aminopropane diacetate | | | | | |
| 1-alkylamino-2-aminopropane | 61791-64-8 | 67302 | 1335 | Microbiocide | Alkyl Amino Propane |
| monoacetate, alkyl derived | | | | | |
| from coconut oil fatty acids | | | | | |
| 1-Alkylamino-3-aminopropane | 68155-43-1 | 67309 | 1972 | Microbiocide, | Alkyl Amino Propane |
| hydroxyacetate, alkyl derived | | | | Soap/Surfactant | |
| from coconut oil fatty acids | | | | | |
| Alkyl (53% C12, 19% C14, | 61791-58-0 | 67305 | 1780 | Microbiocide | Alkyl Amino Propane |
| 8.5% C16, 7% C8, 6.5% C10, | | | | | |
| 6% C18)-1,3-propane diamine | | | | | |
| Alkyl-1,3-propylene diamine | 61790-63-7 | NA 558 | 1380 | Microbiocide, | Alkyl Amino Propane |
| acetate, alkyl derived from | | | | Soap/Surfactant | |
| coconut oil fatty acids | | | | | |
| Alkyl-1,3-propylene diamine | 68155-42-0 | ##### | 1841 | Microbiocide, | Alkyl Amino Propane |
| adipate, alkyl derived from | | | | Soap/Surfactant | |
| coconut oil fatty acids | | | | | |
| Alkyl-1,3-propylene diamine, | NA 287 | NA 559 | 1379 | Microbiocide, | Alkyl Amino Propane |
| alkyl derived from coconut oil | | | | Soap/Surfactant | |
| fatty acids | | | | | |
| N-(2-cyanoethyl)-N-alkyl (C6-C18)- | NA 187 | 67321 | 1957 | Adjuvant | Alkyl Amino Propane |
| 1,3-diamino propane | | | | | |
| N-Alkyl-1,3-propylene diamine | 68188-29-4 | 67307 | 1735 | Microbiocide, | Alkyl Amino Propane |
| monobenzoate, alkyl derived | | | | Soap/Surfactant | |
| from coconut oil fatty acids | | | | | |
| Tall oil fatty acid soap of N- | 61790-55-4 | NA 924 | 3463 | Microbiocide, | Alkyl Amino Propane |
| alkyl (C16-C18) propyl | | | | Insecticide | |
| diamine | | | | | |
| 2,2′-Methylenebis(4,6- | 1940-43-8 | 55004 | NA 455 | Microbiocide | Chlorinated Phenol |
| dichlorophenol) | | | | | |
| 2,2′-Thiobis(4,6- | 97-18-7 | 64201 | NA 539 | N/A | Chlorinated Phenol |
| dichlorophenol) | | | | | |
| 2,2,2-Trichloro-N- | 3583-63-9 | ##### | NA 151 | N/A | Chlorinated Phenol |
| (pentachlorophenyl)acetimidoyl | | | | | |
| chloride | | | | | |
| 2,2-methylene bis(4,6- | 68957-70-0 | 55006 | 1843 | Microbiocide | Chlorinated Phenol |
| dichlorophenol), sodium salt | | | | | |
| 2,3,4,6-tetrachlorophenol, | 53535-27-6 | 63007 | 1725 | Wood | Chlorinated phenol |
| potassium salt | | | | Preservative, | |
| | | | | Fungicide | |
| 2,4,5-trichlorophenol | 95-95-4 | 64210 | 1382 | Microbiocide, | Chlorinated phenol |
| | | | | Algaecide | |
| 2,4,5-Trichlorophenol salt of | 53404-83-4 | 64211 | NA 542 | N/A | Chlorinated Phenol |
| 2,6- | | | | | |
| bis((dimethylamino)methyl)cyclohexanone | | | | | |
| 2,4,5-trichlorophenol, | 35471-43-3 | 64204 | 1050 | Microbiocide | Chlorinated phenol |
| potassium salt | | | | | |
| 2,4,5-trichlorophenol, | NA 1128 | NA 166 | 91050 | Microbiocide | Chlorinated phenol |
| potassium salt, other related | | | | | |
| 2,4,5-trichlorophenol, sodium | 136-32-3 | 64217 | 1656 | Microbiocide | Chlorinated phenol |
| salt | | | | | |
| 2,4,5-Trichlorophenol, sodium | 3784-03-0 | 64220 | NA 545 | Microbiocide, | Chlorinated Phenol |
| salt | | | | Algaecide | |
| 2,4,6-trichlorophenol | 88-06-2 | 64212 | 640 | Microbiocide | Chlorinated phenol |
| 2,4-dichlorophenol | 120-83-2 | NA 105 | 2499 | N/A | Chlorinated phenol |
| 2-(trichlorophenoxy)ethyl | 69633-04-1 | ##### | NA 152 | Herbicide | Chlorinated Phenol |
| hydrogen sulfate | | | | | |
| 2-chloro-4-phenylphenol | 92-04-6 | 62206 | 846 | Microbiocide | Chlorinated Phenol |
| 2-chloro-4-phenylphenol, | 18128-16-0 | 62207 | 1061 | Microbiocide | Chlorinated Phenol |
| potassium salt | | | | | |
| 2-chloro-4-phenylphenol, | 31366-97-9 | 62215 | 1632 | Microbiocide | Chlorinated Phenol |
| sodium salt | | | | | |
| 2-Chlorophenol | 95-57-8 | 62204 | NA 504 | Microbiocide | Chlorinated Phenol |
| 3-chloro-2-biphenylol, sodium | 10605-11-5 | 62213 | NA 505 | Microbiocide | Chlorinated Phenol |
| salt | | | | | |
| 4 & 6-chloro-2-phenylphenol | NA 964 | NA 419 | 1566 | Microbiocide | Chlorinated Phenol |
| 4 & 6-chloro-2-phenylphenol, | NA 965 | NA 420 | 1604 | Microbiocide | Chlorinated Phenol |
| potassium salts, mixed | | | | | |
| 4 & 6-chloro-2-phenylphenol, | NA 963 | NA 413 | 1594 | Microbiocide | Chlorinated Phenol |
| sodium salts, mixed | | | | | |
| 4 & 6-chloro-2-phenylphenol, | NA 1132 | NA 166 | 91566 | Microbiocide | Chlorinated phenol |
| other related | | | | | |
| 4 or 6-chloro-2-phenylphenol | 53537-62-5 | 62216 | NA 507 | Microbiocide | Chlorinated Phenol |
| 4 or 6-chloro-2-phenylphenol, | 53537-63-6 | 62214 | NA 506 | Microbiocide | Chlorinated Phenol |
| diethanolamine salt | | | | | |
| 4,6-dichloro-2-phenylphenol | 5335-24-0 | 64216 | 1062 | Microbiocide | Chlorinated phenol |
| 4,6-dichloro-2-phenylphenol, | 53404-30-1 | 64215 | 1059 | Microbiocide | Chlorinated phenol |
| potassium salt | | | | | |
| 4-chloro-2-cyclopentyl phenol, | 35471-38-6 | 64214 | 1237 | Microbiocide, | Chlorinated phenol |
| potassium salt | | | | Fungicide | |
| 4-chloro-2-cyclopentylphenol | 13347-42-7 | 64202 | 836 | Microbiocide, | Chlorinated phenol |
| | | | | Fungicide | |
| 4-chloro-2-phenylphenol | 607-12-5 | 62208 | 945 | Microbiocide | Chlorinated Phenol |
| 4-chloro-2-phenylphenol, | 53404-21-0 | 62209 | 947 | Microbiocide | Chlorinated Phenol |
| potassium salt | | | | | |
| 4-chloro-2-phenylphenol, | 10605-10-4 | 62212 | 946 | Microbiocide | Chlorinated Phenol |
| sodium salt | | | | | |
| 4-chloro-cyclopentylphenol, | 53404-20-9 | 64218 | 1395 | Microbiocide, | Chlorinated phenol |
| sodium salt | | | | Fungicide | |
| 6-chloro-2-phenylphenol | 85-97-2 | 62210 | 948 | Microbiocide | Chlorinated Phenol |
| 6-chloro-2-phenylphenol, | 18128-17-1 | 62211 | 950 | Microbiocide | Chlorinated Phenol |
| potassium salt | | | | | |
| 6-chloro-2-phenylphenol, | 63992-41-6 | NA 414 | 949 | N/A | Chlorinated phenol |
| sodium salt | | | | | |
| 6-chlorothymol | 89-68-9 | 80403 | NA 775 | N/A | Chlorinated Phenol |
| 6-tert-Butyl-4-chloro-m-cresol | 30894-16-7 | ##### | NA 127 | Microbiocide | Chlorinated Phenol |
| Chlorbisan | 4418-66-0 | 64208 | 127 | Microbiocide | Chlorinated Phenol |
| Chloroxylenol | 88-04-0 | 86801 | 925 | Microbiocide | Chlorinated phenol |
| Copper pentachlorophenate | 15773-35-0 | 63011 | NA 512 | Wood | Chlorinated Phenol, |
| | | | | Preservative, | Inorganic-Copper |
| | | | | Microbiocide, | |
| | | | | Algaecide, | |
| | | | | Fungicide | |
| Dehydroabietylamine | 35109-57-0 | 4202 | NA 72 | Wood | Chlorinated Phenol |
| pentachlorophenate | | | | Preservative, | |
| | | | | Microbiocide, | |
| | | | | Algaecide, | |
| | | | | Fungicide | |
| Dichlorophene | 97-23-4 | 55001 | 206 | Herbicide, | Chlorinated Phenol |
| | | | | Fungicide, | |
| | | | | Microbiocide | |
| Disodium 2,2′- | 3247-34-5 | 44903 | NA 380 | N/A | Chlorinated Phenol |
| methylenebis(3,4,6- | | | | | |
| trichlorophenate) | | | | | |
| Ethylmercury | 22232-28-6 | 41504 | NA 359 | Microbiocide | Organomercury, |
| pentachlorophenate | | | | | Chlorinated Phenol, |
| | | | | | Heavy metal |
| Fenticlor | 97-24-5 | 64209 | NA 541 | Microbiocide | Chlorinated Phenol |
| Hexachlorophene | 70-30-4 | 44901 | 322 | Microbiocide, | Chlorinated Phenol |
| | | | | Fungicide | |
| Hexachlorophene, sodium salt | 5736-15-2 | 44902 | 413 | Microbiocide | Chlorinated Phenol |
| Ortho-benzyl-para- | 120-32-1 | 62201 | 522 | Microbiocide, | Chlorinated phenol |
| chlorophenol | | | | Fungicide | |
| Ortho-benzyl-para- | 35471-49-9 | 62202 | 936 | Microbiocide | Chlorinated phenol |
| chlorophenol, potassium salt | | | | | |
| Ortho-benzyl-para- | 3184-65-4 | 62203 | 937 | Microbiocide, | Chlorinated phenol |
| chlorophenol, sodium salt | | | | Fungicide | |
| Para-chloro-meta-cresol | 59-50-7 | 64206 | 1813 | Microbiocide, | Chlorinated Phenol |
| | | | | Fungicide | |
| para-Chloro-ortho-cresol | 1570-64-5 | 62190 | NA 503 | N/A | Chlorinated Phenol |
| Para-chlorophenol | 106-48-9 | NA 124 | 1572 | N/A | Chlorinated phenol |
| PCP | 87-86-5 | 63001 | 465 | Wood | Chlorinated Phenol |
| | | | | Preservative, | |
| | | | | Microbiocide, | |
| | | | | Algaecide, | |
| | | | | Fungicide, | |
| | | | | Molluscicide, | |
| | | | | Herbicide | |
| PCP, other related | NA 1101 | NA 163 | 90465 | Wood | Chlorinated Phenol |
| | | | | Preservative, | |
| | | | | Microbiocide, | |
| | | | | Algaecide, | |
| | | | | Fungicide, | |
| | | | | Molluscicide | |
| PCP, potassium salt | 7978-73-6 | 63002 | 1049 | Wood | Chlorinated Phenol |
| | | | | Preservative, | |
| | | | | Microbiocide, | |
| | | | | Algaecide, | |
| | | | | Fungicide, | |
| | | | | Molluscicide | |
| PCP, sodium salt | 131-52-2 | 63003 | 540 | Wood | Chlorinated Phenol |
| | | | | Preservative, | |
| | | | | Microbiocide, | |
| | | | | Algaecide, | |
| | | | | Fungicide, | |
| | | | | Molluscicide | |
| PCP, sodium salt, other related | NA 1110 | NA 164 | 90540 | Wood | Chlorinated Phenol |
| | | | | Preservative, | |
| | | | | Microbiocide, | |
| | | | | Algaecide, | |
| | | | | Fungicide, | |
| | | | | Molluscicide | |
| Pentachlorophenol, zinc salt | 2917-32-0 | 63008 | NA 510 | Wood | Chlorinated Phenol, |
| | | | | Preservative, | Inorganic-Zinc |
| | | | | Microbiocide, | |
| | | | | Algaecide, | |
| | | | | Fungicide | |
| Pentachlorophenyl laurate | 3772-94-9 | 63010 | NA 511 | Wood | Chlorinated Phenol |
| | | | | Preservative, | |
| | | | | Microbiocide, | |
| | | | | Algaecide, | |
| | | | | Fungicide | |
| Potassium 2,2′- | 67923-62-0 | 44904 | NA 381 | Microbiocide | Chlorinated Phenol |
| methylenebis(3,4,6- | | | | | |
| trichlorophenate) | | | | | |
| Potassium 2,4,6- | 2591-21-1 | 64219 | NA 544 | Microbiocide | Chlorinated Phenol |
| trichlorophenate | | | | | |
| Potassium ortho-cyclopentyl- | 35471-38-6 | 64214 | 1542 | Microbiocide, | Chlorinated phenol |
| para-chlorophenate | | | | Fungicide | |
| Sodium 2,2′-methylenebis(4- | 10254-48-5 | 55005 | NA 456 | Insecticide, | Chlorinated Phenol |
| chlorophenate) | | | | Fungicide, | |
| | | | | Microbiocide | |
| Sodium chlorophenates | 35535-81-0 | 62205 | 773 | N/A | Chlorinated Phenol |
| Sodium dichlorophenate | NA 1399 | NA 197 | NA 196 | Fungicide, | Chlorinated Phenol |
| | | | | Microbiocide | |
| Sodium p-chloro-m-cresolate | 15733-22-9 | 64205 | NA 540 | Microbiocide | Chlorinated Phenol |
| Tetrachlorophenol | 25167-83-3 | 63004 | 777 | Wood | Chlorinated phenol |
| | | | | Preservative, | |
| | | | | Fungicide | |
| Tetrachlorophenol, alkylamine | NA 1007 | NA 938 | 1401 | Wood | Chlorinated phenol |
| salt | | | | Preservative, | |
| | | | | Fungicide | |
| Tetrachlorophenol, coconut | NA 1008 | NA 939 | 1455 | Wood | Chlorinated phenol |
| amine salt | | | | Preservative, | |
| | | | | Fungicide | |
| Tetrachlorophenol, other | NA 1118 | NA 165 | 90777 | Wood | Chlorinated phenol |
| related | | | | Preservative, | |
| | | | | Fungicide | |
| Tetrachlorophenol, potassium | 53535-27-6 | 63007 | 1753 | Wood | Chlorinated phenol |
| salt | | | | Preservative, | |
| | | | | Fungicide | |
| Tetrachlorophenol, sodium salt | 25567-55-9 | 63005 | 776 | Wood | Chlorinated phenol |
| | | | | Preservative, | |
| | | | | Fungicide | |
| Tetrachlorophenol, sodium salt, | NA 1117 | NA 165 | 90776 | Wood | Chlorinated phenol |
| other related | | | | Preservative, | |
| | | | | Fungicide | |
| Tetrachlorophenols, alkyl* | 137543-70-5 | 63006 | NA 509 | Wood | Chlorinated Phenol |
| amine salt *(as in fatty acids of | | | | Preservative, | |
| coconut oil) | | | | Fungicide | |
| Trichlorophenol | 95-95-4 | 64210 | 1189 | Microbiocide, | Chlorinated phenol |
| | | | | Algaecide | |
| Trichlorophenol, sodium salt | 136-32-3 | 64217 | 960 | Microbiocide, | Chlorinated phenol |
| | | | | Algaecide | |
| Trichlorophenol, sodium salt, | NA 1127 | NA 166 | 90960 | Microbiocide | Chlorinated phenol |
| other related | | | | | |
| Triclosan | 3380-34-5 | 54901 | 1371 | Microbiocide | Chlorinated Phenol |
| Vancide 30-R | 6385-58-6 | 64203 | 612 | Microbiocide | Chlorinated Phenol |
| Zinc 2,4,5-trichlorophenate | 136-24-3 | 64221 | NA 546 | N/A | Chlorinated Phenol, |
| | | | | | Inorganic-Zinc |
| Zinc trichlorophenate | 30143-22-7 | 64213 | NA 543 | Microbiocide | Chlorinated Phenol, |
| | | | | | Inorganic-Zinc |
| 1,3-bis (dihydroxymethyl)-5,5- | 6440-58-0 | ##### | 2298 | Fungicide, | Hydantoin |
| dimethylhydantoin | | | | Herbicide, | |
| | | | | Microbiocide | |
| 1,3-dibromo-5,5- | 77-48-5 | 6317 | 4036 | Microbiocide, | Hydantoin |
| dimethylhydantoin | | | | Fungicide, | |
| | | | | Herbicide | |
| 1,3-dichloro-5,5- | 118-52-5 | 28501 | 37 | Microbiocide, | Hydantoin |
| dimethylhydantoin | | | | Fungicide, | |
| | | | | Herbicide | |
| 1,3-Dichloro-5-ethyl-5- | 89415-87-2 | ##### | 2264 | Microbiocide, | Hydantoin |
| methylhydantoin | | | | Fungicide, | |
| | | | | Herbicide | |
| 1-bromo-3-chloro-5,5-dimethyl | 16079-88-2 | 6315 | 2080 | Microbiocide, | Hydantoin |
| hydantoin | | | | Fungicide, | |
| | | | | Herbicide | |
| 1-Hydroxymethyl-5,5-dimethyl | 116-25-6 | ##### | 2306 | Fungicide, | Hydantoin |
| hydantoin | | | | Herbicide, | |
| | | | | Microbiocide | |
| 3-Bromo-1-chloro-5,5- | 126-06-7 | 6322 | NA 92 | Microbiocide, | Hydantoin |
| dimethylhydantoin | | | | Fungicide, | |
| | | | | Herbicide | |
| 3-Hydroxymethyl-5,5-dimethyl | NA 1478 | NA 220 | NA 219 | Fungicide | Hydantoin |
| hydantoin | | | | | |
| Dimethylol dimethyl hydantoin | 6440-58-0 | ##### | 2524 | Fungicide, | Hydantoin |
| | | | | Herbicide, | |
| | | | | Microbiocide | |
| Acetalized polyvinyl alcohol | NA 1185 | 46926 | NA 166 | Microbiocide | Iodine Compound |
| complexed with iodine | | | | | |
| Alkyl (29% C14, 29% C13, | NA 973 | NA 557 | 2063 | Microbiocide | Iodine Compound |
| 21% C12, 21% C15) | | | | | |
| poly(oxypropylene) poly | | | | | |
| (oxyethylene)-iodine complex | | | | | |
| Alkyl (C12-C15)- | NA 972 | NA 556 | 1734 | Microbiocide | Iodine Compound |
| poly(oxypropylene) | | | | | |
| poly(oxyethylene)-iodine | | | | | |
| complex | | | | | |
| Alkyl(C12-C15) | NA 1195 | 46918 | NA 168 | Microbiocide | Iodine Compound |
| poly(oxypropylene)poly(oxyethylene)- | | | | | |
| iodine complex | | | | | |
| Alkyl* (ethyl cycloimidinium) | NA 1211 | 46906 | NA 169 | Microbiocide | Iodine Compound |
| 3-hydroxy-3-ethyl sodium | | | | | |
| alcoholate, 2-methyl sodium | | | | | |
| carboxylate-tridecyl | | | | | |
| polyoxyethylene ethanol | | | | | |
| Alkyl* dimethyl 3,4- | NA 1198 | 46907 | NA 168 | Microbiocide, | Iodine Compound, |
| dichlorobenzyl ammonium | | | | Microbiocide | Quaternary |
| chloride | | | | | Ammonium |
| polyethoxypolypropoxypolyethoxyethanol- | | | | | Compound |
| iodine complex | | | | | |
| *(50% C12, 30% C14, 1 | | | | | |
| Alkyl* | NA 1214 | 46925 | NA 170 | Microbiocide | Iodine Compound |
| poly(oxypropylene)poly(oxyethylene)- | | | | | |
| iodine complex | | | | | |
| *(43% C10, 30% C14, 12% C12, | | | | | |
| 10% C16, 5% C18) | | | | | |
| Butoxy polypropoxy | 68610-00-4 | 46901 | 1633 | Microbiocide | Iodine Compound |
| polyethoxy ethanol-iodine | | | | | |
| complex | | | | | |
| Diethanolamine myristate- | 53404-40-3 | 46910 | NA 406 | Microbiocide | Iodine Compound |
| iodine complex | | | | | |
| Iodophors | 39392-86-4 | NA 213 | NA 212 | Microbiocide | Iodine Compound |
| Nonyl phenoxy poly | NA 927 | NA 39 | 1610 | Microbiocide | Iodine Compound |
| (oxyethylene) amine sulfonate | | | | | |
| ethanol-iodine | | | | | |
| Nonyl phenoxy | 35860-86-7 | 46903 | 870 | Microbiocide | Iodine Compound, |
| polyoxyethylene ethanol-iodine | | | | | Polyalkyloxy |
| complex | | | | | Compound |
| Octylphenoxy polyethoxy | 53404-04-9 | 46915 | 2172 | Microbiocide, | Iodine Compound |
| ethanol-iodine complex | | | | Fungicide, | |
| | | | | Herbicide | |
| Poly oxyethylene ethanol | 82010-75-1 | 46924 | 2261 | Microbiocide | Iodine Compound |
| monoesters of 5- (and 6-) | | | | | |
| carboxy-4-hexyl-2- | | | | | |
| cyclohexene-1-octanoic acid, | | | | | |
| complex with iodine | | | | | |
| Poly propoxy poly ethoxy | NA 232 | NA 453 | 1828 | Microbiocide | Iodine Compound |
| ethanol, I2 complex | | | | | |
| Polyethoxy polypropoxy | 26617-87-8 | 46904 | 1585 | Microbiocide | Iodine Compound |
| polyethoxy ethanol-iodine | | | | | |
| complex | | | | | |
| Polyethoxy polypropoxy | NA 1286 | 46909 | NA 174 | Microbiocide | Iodine Compound |
| polyethoxy ethanol-iodine | | | | | |
| complex | | | | | |
| Polyethoxypolypropoxypolyethoxyethanol- | NA 1287 | 46913 | NA 175 | Microbiocide | Iodine Compound, |
| n-alkyl (54% C12, | | | | | Quaternary |
| 18% C14, 9% C16, 9% C18, | | | | | Ammonium |
| 5% C10, 5% C8) di(beta- | | | | | Compound |
| hydroxyethyl) benzyl am | | | | | |
| Polyethylene glycol ether of | 68439-47-4 | 46921 | 2121 | Microbiocide | Iodine Compound |
| linear secondary alcohol- | | | | | |
| iodine complex | | | | | |
| Polyvinyl pyrrolidone-iodine | 25655-41-8 | 46914 | 1611 | Microbiocide | Iodine Compound |
| complex | | | | | |
| Sodium dodecylbenzene | 53467-01-9 | 46920 | 1569 | Microbiocide | Iodine Compound |
| sulfonate-iodine complex | | | | | |
| Sodium N-alkyl-N-palmitoyl | NA 290 | NA 562 | 1568 | Microbiocide | Iodine Compound |
| taurate-iodine complex | | | | | |
| Sodium N-cyclohexyl-N- | 53404-81-2 | 46919 | 2089 | Microbiocide | Iodine Compound |
| palmitoyl taurate-iodine | | | | | |
| complex | | | | | |
| Tetraglycine hydroperiodide | 7097-60-1 | 46923 | 1923 | Microbiocide | Iodine Compound |
| Tridecylpoly(ethyleneoxy)ethanol- | 53404-05-0 | 46911 | NA 407 | Microbiocide | Iodine Compound |
| iodine complex | | | | | |
| Triethanolamine octylsulfate- | 82010-77-3 | 46922 | NA 409 | Microbiocide | Iodine Compound |
| iodine complex | | | | | |
| 2-(4-chlorophenylmethyl)- | 26530-09-6 | ##### | NA 125 | Microbiocide | Isothiazolone |
| 3(2H)-isothiazolone | | | | | |
| 2-cyclohexyl-4,5-dichloro-4- | 57063-29-3 | ##### | NA 166 | Insecticide | Isothiazolone |
| isothiazolin-3-one | | | | | |
| 2-cyclohexyl-4,5-dichloro-4- | 57063-29-3 | ##### | NA 938 | Insecticide | Isothiazolone |
| isothioazolin-3-one | | | | | |
| 2-Methyl-3(2H)-isothiazolone, | 57373-20-3 | ##### | NA 895 | Microbiocide | Isothiazolone |
| calcium chloride complex | | | | | |
| 2-methyl-4,5-trimethylene-4- | 82633-79-2 | ##### | 5013 | Microbiocide | Isothiazolone |
| isothiazolin-3-one | | | | | |
| 2-methyl-4-isothiazolin-3-one | 2682-20-4 | ##### | 2039 | Microbiocide | Isothiazolone |
| 3(2H)-chloro-2-methyl-3(2H)- | 57373-19-0 | ##### | NA 894 | Microbiocide | Isothiazolone |
| isothiazolone, calcium chloride | | | | | |
| complex | | | | | |
| 4,5-dichloro-2-n-octyl-3(2H)- | 64359-81-5 | ##### | 3982 | Microbiocide, | Isothiazolone |
| isothiazolone | | | | Antifoulant | |
| 4-chloro-2-n-octyl-3(2H)- | 64359-80-4 | ##### | NA 102 | Microbiocide | Isothiazolone |
| isothiazolone | | | | | |
| 5-chloro-2-((4- | 83542-80-7 | ##### | NA 125 | Microbiocide | Isothiazolone |
| chlorophenyl)methyl)-3(2H)- | | | | | |
| isothiazolone, compd. With 2- | | | | | |
| ((4-chlorophenyl)methyl)- | | | | | |
| 3(2h)-isothiazolone (1:1) | | | | | |
| 5-chloro-2-(4- | 66159-95-3 | ##### | NA 125 | Microbiocide | Isothiazolone |
| chlorophenylmethyl)-3(2H)- | | | | | |
| isothiazolone | | | | | |
| 5-chloro-2-methyl-4- | 26172-55-4 | ##### | 2038 | Microbiocide | Isothiazolone |
| isothiazolin-3-one | | | | | |
| 5-chloro-2-methyl-4- | 55965-87-2 | ##### | NA 896 | Microbiocide | Isothiazolone |
| isothiazolin-3-one calcium | | | | | |
| chloride with 2-methyl-4- | | | | | |
| isothazolin-3-one calcium | | | | | |
| chloride | | | | | |
| Kathon 886 (kathon biocide) | 55965-84-9 | ##### | NA 24 | Microbiocide | Isothiazolone |
| Octhilinone | 26530-20-1 | 99901 | 1881 | Microbiocide, | Isothiazolone |
| | | | | Fungicide | |
| 2,4,6-triisopropyl phenol | 376389 | NA 116 | 3492 | N/A | Phenols |
| 2,4-xylenol | 105-67-9 | 86804 | 638 | Solvent, | Phenols |
| | | | | Fungicide, | |
| | | | | Microbiocide | |
| 2,6-bis (1-methyl heptadecyl)- | 5012-62-4 | NA 106 | 3069 | Microbiocide | Phenols |
| p-cresol | | | | | |
| 2-tert-Amylphenol | 87-26-3 | 64119 | NA 538 | Microbiocide | Phenols |
| 4-Hexylresorcinol | 136-77-6 | 71403 | NA 671 | N/A | Phenols |
| 4-Octylresorcinol | 6565-70-4 | 71402 | NA 670 | N/A | Phenols |
| 5-isopropyl-o-cresol | 499-75-2 | 22104 | 1877 | Microbiocide | Phenols |
| Butylated hydroxy toluene | 128-37-0 | 22105 | 1309 | Preservative | Phenols |
| Carbolic acid | 108-95-2 | 64001 | 107 | Microbiocide, | Phenols |
| | | | | Nematicide | |
| Coal tar phenols | 1319-77-3 | 22101 | 1720 | N/A | Petroleum derivative, |
| | | | | | Phenols |
| Cresol | 1319-77-3 | NA 219 | NA 218 | Insect | Phenols |
| | | | | Repellent, | |
| | | | | Microbiocide | |
| Diisobutyl cresoxy ethoxy | 53466-87-8 | 69155 | 1643 | Microbiocide | Phenols |
| ethyl dimethyl benzyl | | | | | |
| ammonium chloride-ortho- | | | | | |
| phenylphenol ethoxy nonyl | | | | | |
| phenol complex | | | | | |
| Diisopropyl cresol | 31291-59-5 | 22107 | NA 176 | Microbiocide | Phenols |
| Diisopropyl phenols | NA 170 | NA 329 | 3638 | N/A | Phenols |
| Hydroxymercury cresol | 12379-66-7 | 46001 | NA 390 | Microbiocide | Inorganic-Mercury, |
| | | | | | Heavy metal, Phenols |
| Isopropyl cresol | 499-75-2 | 22104 | 1161 | Microbiocide | Phenols |
| Isopropyl phenol | 25168-06-3 | NA 155 | 3256 | Microbiocide | Phenols |
| Meta-cresol | 108-39-4 | 22102 | 378 | Microbiocide | Phenols |
| Nonyl phenol | 25154-52-3 | NA 44 | 3304 | Adjuvant | Phenols |
| o-cresol | 95-48-7 | NA 374 | 3113 | Microbiocide | Phenols |
| o-Phenylphenol, alkyl* amine- | 82010-74-0 | 64107 | NA 533 | Microbiocide | Phenols, Inorganic- |
| zinc salt of *(100% C18) | | | | | Zinc |
| o-Phenylphenol, | 53404-71-0 | 64109 | NA 534 | Microbiocide | Phenols |
| tetradecylamine salt | | | | | |
| Octylphenol | 27193-28-8 | 64118 | NA 537 | N/A | Phenols |
| Ortho-phenylphenol | 90-43-7 | 64103 | 448 | Microbiocide | Phenols |
| Ortho-phenylphenol, | 52704-98-0 | 64116 | 890 | Microbiocide | Phenols |
| ammonium salt | | | | | |
| Ortho-phenylphenol, potassium | 13707-65-8 | 64108 | 935 | Microbiocide | Phenols |
| salt | | | | | |
| Ortho-phenylphenol, sodium | 132-27-4 | 64104 | 248 | Microbiocide | Phenols |
| salt | | | | | |
| p-cresol | 106-44-5 | NA 375 | 3114 | Microbiocide | Phenols |
| p-Cresyl acetate | 140-39-6 | NA 186 | NA 186 | Microbiocide | Phenols |
| Para-tert-amylphenol | 80-46-6 | 64101 | 900 | Microbiocide | Phenols |
| Para-tert-amylphenol, | 53404-18-5 | 64111 | 1056 | Microbiocide | Phenols |
| potassium salt | | | | | |
| Para-tert-butylphenol | 98-54-4 | 64113 | 1000 | Microbiocide | Phenols |
| Phenol, dodecyl-, mixed | 27193-86-8 | NA 656 | 3323 | N/A | Phenols |
| isomers | | | | | |
| Phenol, ferrous salt | NA 686 | NA 126 | 1862 | N/A | Phenols |
| Phenols | 64743-03-9 | NA 189 | NA 189 | Microbiocide, | Phenols |
| | | | | Herbicide | |
| Polyvinylpyrrolidone- | 53403-98-8 | 64110 | NA 535 | Microbiocide | Phenols |
| orthophenylphenol complex | | | | | |
| Propoxur phenol | 4812-20-8 | ##### | NA 118 | Breakdown | Phenols |
| | | | | product | |
| Resorcinol | 108-46-3 | 71401 | 3375 | N/A | Phenols |
| Sodium cresolate | 34689-46-8 | ##### | NA 142 | Microbiocide | Phenols |
| Sodium para-tert-amylphenate | 31366-95-7 | 64112 | 1541 | Microbiocide, | Phenols |
| | | | | Fungicide | |
| Sodium para-tert-butylphenate | 5787-50-8 | 64115 | 1540 | Microbiocide | Phenols |
| Sodium phenate | 139-02-6 | 64002 | 2164 | Microbiocide | Phenols |
| Thymol | 89-83-8 | 80402 | 991 | Dye | Phenols |
| Xylenol (unspec. Or mixed | 1300-71-6 | 86805 | NA 837 | N/A | Phenols |
| from 086804) | | | | | |
| 2-(2-(p-(diisobutyl) phenoxy) | 121-54-0 | 69122 | 66 | Microbiocide | Quaternary |
| ethoxy) ethyl dimethyl | | | | | Ammonium |
| ammonium chloride | | | | | Compound |
| 2-(2-(p-(diisobutyl) phenoxy) | 121-54-0 | 69164 | NA 166 | Microbiocide | Quaternary |
| ethoxy) ethyl dimethyl | | | | | Ammonium |
| ammonium chloride | | | | | Compound |
| 3-(trimethoxysilyl) propyl | 27668-52-6 | ##### | 2127 | Microbiocide | Quaternary |
| dimethyl octadecyl ammonium | | | | | Ammonium |
| chloride | | | | | Compound |
| 3-alkoxy (C12-C15)-2- | 68187-63-3 | ##### | 2020 | N/A | Quaternary |
| hydroxypropyl trimethyl | | | | | Ammonium |
| ammonium chloride | | | | | Compound |
| Acylamido alkylbenzyl | NA 993 | NA 705 | 1442 | Algaecide, | Quaternary |
| dimethyl ammonium chloride | | | | Microbiocide | Ammonium |
| | | | | | Compound |
| Alkenyl (90% C18, 10% C16) | NA 1313 | NA 123 | 1243 | Microbiocide, | Quaternary |
| dimethyl ethyl ammonium | | | | Adjuvant | Ammonium |
| bromide | | | | | Compound |
| Alkenyl* dimethyl ethyl | 6458-13-5 | 69102 | NA 620 | Algaecide, | Quaternary |
| ammonium bromide | | | | Microbiocide | Ammonium |
| *(90% C18′, 10% C16′) | | | | | Compound |
| Alkenyl* dimethyl ethyl | NA 1187 | 69198 | NA 167 | Microbiocide | Quaternary |
| ammonium bromide | | | | | Ammonium |
| *(90% C18′, 10% C16′) | | | | | Compound |
| Alkyl (100% C14) dimethyl | 139-08-2 | 69107 | 2373 | Algaecide, | Quaternary |
| benzyl ammonium chloride | | | | Microbiocide | Ammonium |
| | | | | | Compound |
| Alkyl (47% C12, 18% C14, | NA 986 | 69143 | 1970 | Algaecide, | Quaternary |
| 10% C18, 10% C16, 15% C8-C10) | | | | Microbiocide | Ammonium |
| dimethylbenzyl | | | | | Compound |
| ammonium chloride | | | | | |
| Alkyl (50% C12, 30% C14, | 8023-53-8 | 69110 | 2027 | Algaecide, | Quaternary |
| 17% C16, 3% C18) dimethyl | | | | Microbiocide | Ammonium |
| dichlorobenzyl ammonium | | | | | Compound |
| chloride | | | | | |
| Alkyl (50% C12, 30% C14, | NA 987 | 69143 | 1850 | Algaecide, | Quaternary |
| 17% C16, 3% C18) | | | | Microbiocide | Ammonium |
| dimethylbenzyl ammonium | | | | | Compound |
| chloride | | | | | |
| Alkyl (50% C12, 30% C14, | 8045-21-4 | 69111 | 1849 | Algaecide, | Quaternary |
| 17% C16, 3% C18) | | | | Microbiocide | Ammonium |
| dimethylethylbenzyl | | | | | Compound |
| ammonium chloride | | | | | |
| Alkyl (50% C14, 40% C12, | NA 988 | 69143 | 1846 | Algaecide, | Quaternary |
| 10% C16) dimethylbenzyl | | | | Microbiocide | Ammonium |
| ammonium chloride | | | | | Compound |
| Alkyl (50% C14, 40% C12, | 68989-01-5 | 69171 | 324 | Algaecide, | Quaternary |
| 10% C16) dimethylbenzyl | | | | Microbiocide | Ammonium |
| ammonium saccharinate | | | | | Compound |
| Alkyl (53% C12, 19% C14, | 61789-71-7 | 69140 | 3535 | Algaecide, | Quaternary |
| 8.5% C16, 7% C8, 6.5% C10, | | | | Microbiocide | Ammonium |
| 6% C18) dimethyl | | | | | Compound |
| benzylammonium chloride | | | | | |
| Alkyl (58% C14, 28% C16, | NA 989 | 69143 | 1912 | Algaecide, | Quaternary |
| 14% C12) dimethylbenzyl | | | | Microbiocide | Ammonium |
| ammonium chloride | | | | | Compound |
| Alkyl (58% C18, 40% C16, | 68391-03-7 | 69163 | 2228 | Microbiocide | Quaternary |
| 1% C14, 1% C12) trimethyl | | | | | Ammonium |
| ammonium chloride | | | | | Compound |
| Alkyl (60% C14, 25% C12, | 68424-85-1 | 69105 | 1956 | Algaecide, | Quaternary |
| 15% C16) dimethylbenzyl | | | | Microbiocide | Ammonium |
| ammonium chloride | | | | | Compound |
| Alkyl (60% C14, 30% C16, | NA 976 | 69143 | 1847 | Algaecide, | Quaternary |
| 5% C12, 5% C18) | | | | Microbiocide | Ammonium |
| dimethylbenzyl ammonium | | | | | Compound |
| chloride | | | | | |
| Alkyl (61% C12, 23% C14, | 68989-00-4 | ##### | 1960 | Algaecide, | Quaternary |
| 11% C16, 3% C10, 2% C8) | | | | Microbiocide | Ammonium |
| dimethylbenzyl ammonium | | | | | Compound |
| chloride | | | | | |
| Alkyl (61% C12, 23% C14, | NA 977 | 69143 | 1975 | Algaecide, | Quaternary |
| 11% C16, 5% C18) dimethyl | | | | Microbiocide | Ammonium |
| benzyl ammonium chloride | | | | | Compound |
| Alkyl (61% C12, 23% C14, | NA 978 | 69143 | 2060 | Algaecide, | Quaternary |
| 11% C16, 5% C8, C10, C18) | | | | Microbiocide | Ammonium |
| dimethylbenzyl ammonium | | | | | Compound |
| chloride | | | | | |
| Alkyl | 61789-71-7 | 69140 | 2375 | Algaecide, | Quaternary |
| (61% C12, 23% C14, 11% C16, 2.5% | | | | Microbiocide | Ammonium |
| C8 & C10, 2.5% C18) | | | | | Compound |
| dimethyl benzyl ammonium | | | | | |
| chloride | | | | | |
| Alkyl (65% C12, 25% C14, | NA 979 | 69143 | 1971 | Algaecide, | Quaternary |
| 10% C16) dimethylbenzyl | | | | Microbiocide | Ammonium |
| ammonium chloride | | | | | Compound |
| Alkyl (67% C12, 25% C14, | NA 980 | 69143 | 1853 | Algaecide, | Quaternary |
| 7% C16, 1% C8, C10, C18) | | | | Microbiocide | Ammonium |
| dimethylbenzyl ammonium | | | | | Compound |
| chloride | | | | | |
| Alkyl (68% C12, 32% C14) | NA 1190 | ##### | NA 167 | Algaecide, | Quaternary |
| dimethyl dimethylbenzyl | | | | Microbiocide | Ammonium |
| ammonium chloride | | | | | Compound |
| Alkyl (68% C12, 32% C14) | 85409-23-0 | 69154 | 1854 | Algaecide, | Quaternary |
| dimethylethylbenzyl | | | | Fungicide, | Ammonium |
| ammonium chloride | | | | Microbiocide | Compound |
| Alkyl (70% C18, 27% C16, | 68391-03-7 | 69163 | 10 | Microbiocide | Quaternary |
| 3% C14) trimethyl ammonium | | | | | Ammonium |
| chloride | | | | | Compound |
| Alkyl (90% C14, 5% C12, | 8023-53-8 | 69110 | 1909 | Algaecide, | Quaternary |
| 5% C16) dimethyl | | | | Microbiocide | Ammonium |
| dichlorobenzyl ammonium | | | | | Compound |
| chloride | | | | | |
| Alkyl (90% C14, 5% C12, | 68527-84-4 | 69146 | 2057 | Algaecide, | Quaternary |
| 5% C16) dimethyl ethyl | | | | Microbiocide | Ammonium |
| ammonium bromide | | | | | Compound |
| Alkyl (90% C14, 5% C12, | NA 981 | 69143 | 1977 | Algaecide, | Quaternary |
| 5% C16) dimethylbenzyl | | | | Microbiocide | Ammonium |
| ammonium chloride | | | | | Compound |
| Alkyl (93% C14, 4% C12, | NA 1031 | 69143 | 1991 | Algaecide, | Quaternary |
| 3% C16) dimethylbenzyl | | | | Microbiocide | Ammonium |
| ammonium chloride | | | | | Compound |
| Alkyl (95% C14, 3% C12, | 68424-85-1 | 69105 | 3855 | Algaecide, | Quaternary |
| 2% C16) dimethyl benzyl | | | | Microbiocide | Ammonium |
| ammonium chloride | | | | | Compound |
| Alkyl (95% C14, 3% C12, | NA 982 | 69184 | 2152 | Algaecide, | Quaternary |
| 2% C16) dimethyl benzyl | | | | Microbiocide | Ammonium |
| ammonium chloride dihydrate | | | | | Compound |
| Alkyl (95% C14, 3% C12, | NA 1322 | NA 583 | 3897 | Algaecide, | Quaternary |
| 2% C16) dimethyl benzyl | | | | Microbiocide | Ammonium |
| ammonium chloride | | | | | Compound |
| monohydrate | | | | | |
| Alkyl (98% C12, 2% C14) | 53516-75-9 | 69112 | 1839 | Algaecide, | Quaternary |
| dimethyl 1-naphthylmethyl | | | | Microbiocide | Ammonium |
| ammonium chloride | | | | | Compound |
| Alkyl (C10-16) dimethyl amine | 61788-90-7 | NA 401 | 3896 | Algaecide, | Quaternary |
| oxide | | | | Microbiocide | Ammonium |
| | | | | | Compound |
| Alkyl (C14, C12, C16) | NA 985 | NA 587 | 2374 | Algaecide, | Quaternary |
| dimethyl benzyl ammonium | | | | Microbiocide | Ammonium |
| chloride | | | | | Compound |
| Alkyl (C8-C18) bis (2- | 61789-68-2 | ##### | 2031 | Microbiocide | Quaternary |
| hydroxyethyl) benzyl | | | | | Ammonium |
| ammonium chloride | | | | | Compound |
| Alkyl (C8-C18) bis (2- | NA 1196 | 69148 | NA 168 | Algaecide, | Quaternary |
| hydroxyethyl) benzyl | | | | Microbiocide | Ammonium |
| ammonium chloride | | | | | Compound |
| Alkyl dimethyl cumenyl | NA 983 | NA 584 | 1919 | Algaecide, | Quaternary |
| ammonium chloride | | | | Microbiocide | Ammonium |
| | | | | | Compound |
| Alkyl dimethyl ethyl benzyl | NA 974 | NA 580 | 1002 | Microbiocide | Quaternary |
| ammonium cyclohexyl | | | | | Ammonium |
| sulfonate | | | | | Compound |
| Alkyl dimethyl ethylbenzyl | 71808-54-3 | 69135 | 1722 | Algaecide, | Quaternary |
| ammonium cyclohexyl | | | | Microbiocide | Ammonium |
| sulfamate | | | | | Compound |
| Alkyl dimethyl isopropyl | NA 975 | 69188 | 884 | Microbiocide | Quaternary |
| benzyl ammonium chloride | | | | | Ammonium |
| | | | | | Compound |
| Alkyl monoethyoxyethanol, | NA 305 | NA 577 | 1045 | Microbiocide | Quaternary |
| diethoxyethyl benzyl | | | | | Ammonium |
| ammonium chloride | | | | | Compound |
| Alkyl morpholinium | NA 1523 | NA 223 | NA 223 | Fungicide | Quaternary |
| ethosulphate (1% C14, 22% | | | | | Ammonium |
| C16, 77% C18) | | | | | Compound |
| Alkyl trimethyl ammonium | 68424-92-0 | ##### | 1827 | Microbiocide | Quaternary |
| bromides | | | | | Ammonium |
| | | | | | Compound |
| Alkyl(68% C12, | NA 1068 | NA 159 | 5126 | Algaecide | Quaternary |
| 32% C14)dimethyl | | | | | Ammonium |
| dimethylbenzyl ammonium | | | | | Compound |
| chloride | | | | | |
| Alkyl* bis(2- | 91721-81-2 | ##### | NA 113 | N/A | Quaternary |
| hydroxyethyl)benzyl | | | | | Ammonium |
| ammonium chloride | | | | | Compound |
| *(57% C10, 20% C12, 11% C14, | | | | | |
| 9% C18, 2% C16, 1% C8) | | | | | |
| Alkyl* dimethyl 3,4- | NA 1197 | ##### | NA 168 | Algaecide, | Quaternary |
| dichlorobenzyl ammonium | | | | Microbiocide | Ammonium |
| chloride *(42% C12, 27% C14, | | | | | Compound |
| 13% C16, 18% C8-C18) | | | | | |
| Alkyl* dimethyl 3,4- | 68989-02-6 | ##### | NA 112 | Algaecide, | Quaternary |
| dichlorobenzyl ammonium | | | | Microbiocide | Ammonium |
| chloride *(50% C14, 40% C12, | | | | | Compound |
| 10% C16) | | | | | |
| Alkyl* dimethyl 3,4- | 92129-28-7 | 69182 | NA 648 | Algaecide, | Quaternary |
| dichlorobenzyl ammonium | | | | Microbiocide | Ammonium |
| chloride *(55% C14, 23% C12, | | | | | Compound |
| 20% C16, 2% C18) | | | | | |
| Alkyl* dimethyl 3,4- | 68989-02-6 | ##### | NA 124 | Algaecide, | Quaternary |
| dichlorobenzyl ammonium | | | | Microbiocide | Ammonium |
| chloride *(60% C14, 30% C16, | | | | | Compound |
| 10% C12) | | | | | |
| Alkyl* dimethyl 3,4- | 68989-02-6 | 69180 | NA 646 | Algaecide, | Quaternary |
| dichlorobenzyl ammonium | | | | Microbiocide | Ammonium |
| chloride *(60% C12, 25% C14, | | | | | Compound |
| 15% C16) | | | | | |
| Alkyl* dimethyl 3,4- | 68989-02-6 | ##### | NA 113 | Algaecide, | Quaternary |
| dichlorobenzyl ammonium | | | | Microbiocide | Ammonium |
| chloride *(63% C12, 23% C14, | | | | | Compound |
| 14% C16) | | | | | |
| Alkyl* dimethyl 3,4- | 92129-28-7 | 69196 | NA 653 | Algaecide, | Quaternary |
| dichlorobenzyl ammonium | | | | Microbiocide | Ammonium |
| chloride *(65% C12, 22% C14, | | | | | Compound |
| 10% C16, 3% C18) | | | | | |
| Alkyl* dimethyl 3,4- | 68989-02-6 | ##### | NA 114 | Algaecide, | Quaternary |
| dichlorobenzyl ammonium | | | | Microbiocide | Ammonium |
| chloride *(68% C12, 27% C14, | | | | | Compound |
| 5% C16) | | | | | |
| Alkyl* dimethyl 3,4- | 68568-47-8 | ##### | NA 124 | Algaecide, | Quaternary |
| dichlorobenzyl ammonium | | | | Microbiocide | Ammonium |
| chloride *(75% C12, 25% C14) | | | | | Compound |
| Alkyl* dimethyl 3,4- | 68989-02-6 | ##### | NA 124 | Algaecide, | Quaternary |
| dichlorobenzyl ammonium | | | | Microbiocide | Ammonium |
| chloride *(75% C12, 25% C14) | | | | | Compound |
| Alkyl* dimethyl 3,4- | 68989-02-6 | 69145 | NA 635 | Algaecide, | Quaternary |
| dichlorobenzyl ammonium | | | | Microbiocide | Ammonium |
| chloride *(90% C14, 5% C16, | | | | | Compound |
| 5% C12) | | | | | |
| Alkyl* dimethyl 3,4- | 68989-02-6 | ##### | NA 115 | Algaecide, | Quaternary |
| dichlorobenzyl ammonium | | | | Microbiocide | Ammonium |
| chloride *(C12 65%, C14 25%, | | | | | Compound |
| C16 10%) | | | | | |
| Alkyl* dimethyl 3,4- | NA 1198 | 46907 | NA 168 | Microbiocide, | Iodine Compound, |
| dichlorobenzyl ammonium | | | | Microbiocide | Quaternary |
| chloride | | | | | Ammonium |
| polyethoxypolypropoxypolyethoxyethanol- | | | | | Compound |
| iodine complex | | | | | |
| *(50% C12, 30% C14, 1 | | | | | |
| Alkyl* dimethyl benzyl | 71011-24-0 | ##### | NA 113 | Algaecide, | Quaternary |
| ammonium bentonite *(as in | | | | Microbiocide | Ammonium |
| fatty acids of tallow) | | | | | Compound |
| Alkyl* dimethyl benzyl | 122-18-9 | 69108 | NA 623 | Algaecide, | Quaternary |
| ammonium chloride *(100% | | | | Microbiocide | Ammonium |
| C16) | | | | | Compound |
| Alkyl* dimethyl benzyl | 122-19-0 | ##### | NA 114 | Algaecide, | Quaternary |
| ammonium chloride *(100% | | | | Microbiocide | Ammonium |
| C18) | | | | | Compound |
| Alkyl* dimethyl benzyl | 68391-01-5 | 69195 | NA 652 | Algaecide, | Quaternary |
| ammonium chloride | | | | Microbiocide | Ammonium |
| *(41% C14, 28% C12, 19% C18, | | | | | Compound |
| 12% C16) | | | | | |
| Alkyl* dimethyl benzyl | NA 1199 | 69168 | NA 168 | Algaecide, | Quaternary |
| ammonium chloride | | | | Microbiocide | Ammonium |
| *(47% C12, 18% C14, 15%(C5-C15), | | | | | Compound |
| 10% C18, 10% C16) | | | | | |
| Alkyl* dimethyl benzyl | 8001-54-5 | 69106 | NA 622 | Microbiocide, | Quaternary |
| ammonium chloride | | | | Algaecide, | Ammonium |
| *(50% C12, 30% C14, 17% C16, | | | | Herbicide | Compound |
| 3% C18) | | | | | |
| Alkyl* dimethyl benzyl | 68391-01-5 | ##### | NA 113 | Algaecide, | Quaternary |
| ammonium chloride *(55% | | | | Microbiocide | Ammonium |
| C16, 27% C12, 16% C14, 2% | | | | | Compound |
| C18) | | | | | |
| Alkyl* dimethyl benzyl | 68391-01-5 | 69169 | NA 641 | Algaecide, | Quaternary |
| ammonium chloride | | | | Microbiocide | Ammonium |
| *(55% C16, 20% C14, 20% C12, | | | | | Compound |
| 5% C18) | | | | | |
| Alkyl* dimethyl benzyl | NA 1200 | 69141 | NA 168 | Algaecide, | Quaternary |
| ammonium chloride | | | | Microbiocide | Ammonium |
| *(58% C14, 28% C16, 14% C12) | | | | | Compound |
| Alkyl* dimethyl benzyl | 68424-85-1 | 69161 | NA 168 | Algaecide, | Quaternary |
| ammonium chloride *(60% | | | | Microbiocide | Ammonium |
| C14, 30% C16, 10% C12) | | | | | Compound |
| Alkyl* dimethyl benzyl | NA 1201 | 69137 | NA 168 | Algaecide, | Quaternary |
| ammonium chloride | | | | Microbiocide | Ammonium |
| *(60% C14, 25% C12, 15% C16) | | | | | Compound |
| Alkyl* dimethyl benzyl | 53516-76-0 | 69104 | NA 621 | Algaecide, | Quaternary |
| ammonium chloride | | | | Microbiocide | Ammonium |
| *(60% C14, 30% C16, 5% C18, | | | | | Compound |
| 5% C12) | | | | | |
| Alkyl* dimethyl benzyl | 68391-01-5 | 69189 | NA 650 | Algaecide, | Quaternary |
| ammonium chloride *(61% | | | | Microbiocide | Ammonium |
| C12, 23% C14, 11% C16, 5% | | | | | Compound |
| C18) | | | | | |
| Alkyl* dimethyl benzyl | NA 1202 | ##### | NA 168 | Algaecide, | Quaternary |
| ammonium chloride *(61% | | | | Microbiocide | Ammonium |
| C12, 23% 014, 11% C16, 5% | | | | | Compound |
| C18) | | | | | |
| Alkyl* dimethyl benzyl | NA 1203 | 69199 | NA 169 | Algaecide, | Quaternary |
| ammonium chloride | | | | Microbiocide | Ammonium |
| *(65% C12, 23% C14, 12% C16) | | | | | Compound |
| Alkyl* dimethyl benzyl | 68424-85-1 | 69157 | NA 164 | Algaecide, | Quaternary |
| ammonium chloride | | | | Microbiocide | Ammonium |
| *(65% C12, 25% C14, 10% C16) | | | | | Compound |
| Alkyl* dimethyl benzyl | 68391-01-5 | 69175 | NA 644 | Algaecide, | Quaternary |
| ammonium chloride | | | | Microbiocide | Ammonium |
| *(67% C12, 25% C14, 7% C16, | | | | | Compound |
| 1% C18) | | | | | |
| Alkyl* dimethyl benzyl | NA 1204 | ##### | NA 169 | Algaecide, | Quaternary |
| ammonium chloride | | | | Microbiocide | Ammonium |
| *(67% C12, 25% C14, 7% C16, | | | | | Compound |
| 1% C8, C10, and C18) | | | | | |
| Alkyl* dimethyl benzyl | NA 1205 | 69142 | NA 169 | Microbiocide | Quaternary |
| ammonium chloride | | | | | Ammonium |
| *(67% C12, 27% C14, 6% C16) | | | | | Compound |
| Alkyl* dimethyl benzyl | NA 1206 | ##### | NA 169 | Algaecide, | Quaternary |
| ammonium chloride *(68% | | | | Microbiocide | Ammonium |
| C12, 25% C14, 7% C16) | | | | | Compound |
| Alkyl* dimethyl benzyl | NA 1207 | 69194 | NA 169 | Algaecide, | Quaternary |
| ammonium chloride | | | | Microbiocide | Ammonium |
| *(90% C14, 5% C12, 5% C16) | | | | | Compound |
| Alkyl* dimethyl benzyl | 68424-85-1 | 69158 | NA 164 | Algaecide, | Quaternary |
| ammonium chloride | | | | Microbiocide | Ammonium |
| *(93% C14, 4% C12, 3% C16) | | | | | Compound |
| Alkyl* dimethyl benzyl | 68607-20-5 | ##### | NA 114 | Algaecide, | Quaternary |
| ammonium chloride *(95% | | | | Microbiocide | Ammonium |
| C16, 5% C18) | | | | | Compound |
| Alkyl* dimethyl benzyl | NA 1208 | ##### | NA 169 | Algaecide, | Quaternary |
| ammonium chloride *(as in | | | | Microbiocide | Ammonium |
| fatty acids of coconut oil) | | | | | Compound |
| Alkyl* dimethyl benzyl | NA 1209 | ##### | NA 169 | Algaecide, | Quaternary |
| ammonium chloride *(C8-18) | | | | Microbiocide | Ammonium |
| | | | | | Compound |
| Alkyl* dimethyl benzyl | 61790-41-8 | ##### | NA 114 | Algaecide, | Quaternary |
| ammonium ion alkyl** amine | | | | Microbiocide | Ammonium |
| *(C12, C14, C16) **(C10, | | | | | Compound |
| C12, C14, C16) | | | | | |
| Alkyl* dimethyl | 8045-22-5 | 69109 | NA 624 | Algaecide, | Quaternary |
| dimethylbenzyl ammonium | | | | Microbiocide | Ammonium |
| chloride *(50% C12, 30% C14, | | | | | Compound |
| 17% C16, 3% C18) | | | | | |
| Alkyl* dimethyl ethyl | 134595-54-3 | 69186 | NA 649 | Algaecide, | Quaternary |
| ammonium bromide *(85% | | | | Microbiocide | Ammonium |
| C16, 15% C18) | | | | | Compound |
| Alkyl* dimethyl ethyl | 61788-99-6 | ##### | NA 113 | Algaecide, | Quaternary |
| ammonium bromide *(mixed | | | | Microbiocide | Ammonium |
| alkyl and alkenyl groups as in | | | | | Compound |
| fatty acids of soybean oil) | | | | | |
| Alkyl* dimethyl ethylbenzyl | 68956-79-6 | 69167 | NA 640 | Algaecide, | Quaternary |
| ammonium chloride | | | | Microbiocide | Ammonium |
| *(60% C14, 30% C16, 5% C12, | | | | | Compound |
| 5% C18) | | | | | |
| Alkyl* dimethyl ethylbenzyl | 37335-68-5 | 69135 | NA 633 | Algaecide, | Quaternary |
| ammonium | | | | Microbiocide | Ammonium |
| cyclohexylsulfamate | | | | | Compound |
| *(50% C12, 30% C14, 17% C16, | | | | | |
| 3% C18) | | | | | |
| Alkyl* trimethyl ammonium | 1119-97-7 | 69153 | NA 637 | Microbiocide | Quaternary |
| bromide *(95% C14, 5% C16) | | | | | Ammonium |
| | | | | | Compound |
| Alkyl* trimethyl ammonium | 112-00-5 | ##### | NA 113 | Microbiocide | Quaternary |
| chloride *(70% C12, 30% C14) | | | | | Ammonium |
| | | | | | Compound |
| Alkyl* trimethyl ammonium | 68002-63-1 | 69197 | NA 654 | Microbiocide | Quaternary |
| chloride *(70% C18, 27% C16, | | | | | Ammonium |
| 3% C14) | | | | | Compound |
| Alkyl* trimethyl ammonium | 68002-62-0 | 69151 | NA 636 | Microbiocide | Quaternary |
| chloride *(90% C18, 10% C16) | | | | | Ammonium |
| | | | | | Compound |
| Alkyl* trimethyl ammonium | 61790-41-8 | ##### | NA 115 | Microbiocide | Quaternary |
| chloride *(alkyl as in fatty | | | | | Ammonium |
| acids of soybean oil) | | | | | Compound |
| Alkyl* trimethyl ammonium | 61789-18-2 | ##### | NA 109 | Microbiocide | Quaternary |
| chloride *(as in fatty acids of | | | | | Ammonium |
| coconut oil) | | | | | Compound |
| Alkyl* trimethyl ammonium | 68002-61-9 | 69139 | NA 634 | Microbiocide | Quaternary |
| chloride *derived from | | | | | Ammonium |
| cottonseed oil | | | | | Compound |
| Alkyl*-5-hydroxy-4-oxo- | NA 1189 | ##### | NA 167 | Microbiocide | Quaternary |
| 2(4H)-pyranylmethyl dimethyl | | | | | Ammonium |
| ammonium chloride *(49% | | | | | Compound |
| C12, 17% C14, 17% C10, 10% | | | | | |
| C18, 9% C16, 8% C8) | | | | | |
| Alkyl*-N,N-bis(2- | 70750-47-9 | ##### | NA 114 | N/A | Quaternary |
| hydroxyethyl)methyl | | | | | Ammonium |
| ammonium chloride *(53% | | | | | Compound |
| C12, 19% C14, 8.5% C16, | | | | | |
| 7.0% C8, 6.5% C10, 6.0% | | | | | |
| C18) | | | | | |
| Alkyl*-N-ethyl morpholinium | NA 1213 | 69147 | NA 170 | Fungicide, | Quaternary |
| ethyl sulfate *(66% C18, | | | | Algaecide | Ammonium |
| 25% C16, 8% C18′, 1% C14) | | | | | Compound |
| Alkyldimethylbenzyl | NA 1368 | NA 194 | NA 193 | Microbiocide, | Quaternary |
| ammonium chloride | | | | Herbicide | Ammonium |
| | | | | | Compound |
| Alkyldimethylethylbenzylammonium | NA 1369 | NA 194 | NA 193 | Microbiocide, | Quaternary |
| chloride | | | | Herbicide | Ammonium |
| | | | | | Compound |
| Alkyltrimethyl ammonium | NA 1370 | NA 194 | NA 193 | Microbiocide, | Quaternary |
| chloride | | | | Fungicide | Ammonium |
| | | | | | Compound |
| Alkyltrimethylbenzyl | NA 1371 | NA 194 | NA 193 | Herbicide | Quaternary |
| ammonium chloride | | | | | Ammonium |
| | | | | | Compound |
| Benzyl dimethyl octadecyl | 122-19-0 | ##### | 1186 | Microbiocide | Quaternary |
| ammonium chloride | | | | | Ammonium |
| | | | | | Compound |
| Benzyl((dodecylcarbamoyl)methyl) | 100-95-8 | 69159 | NA 638 | Microbiocide | Quaternary |
| dimethyl ammonium | | | | | Ammonium |
| chloride | | | | | Compound |
| Benzyl-C12-14-alkyldimethyl | 134058-54-1 | 69192 | 5069 | Microbiocide | Quaternary |
| quaternary ammonium | | | | | Ammonium |
| compounds | | | | | Compound |
| Cetyl dimethyl ethyl | 124-03-8 | 69156 | 122 | Microbiocide | Quaternary |
| ammonium bromide | | | | | Ammonium |
| | | | | | Compound |
| Cetyl trimethyl ammonium | 57-09-0 | 69117 | 116 | Microbiocide, | Quaternary |
| bromide | | | | Herbicide | Ammonium |
| | | | | | Compound |
| Cetyl trimethyl ammonium | 112-02-7 | 69133 | NA 632 | Microbiocide | Quaternary |
| chloride | | | | | Ammonium |
| | | | | | Compound |
| Cetylcide (95% C16, 3% C14, | 124-03-8 | 69156 | 2061 | Microbiocide | Quaternary |
| 2% C12) | | | | | Ammonium |
| | | | | | Compound |
| Chlormequat | 7003-89-6 | NA 216 | NA 215 | Plant Growth | Quaternary |
| | | | | Regulator | Ammonium |
| | | | | | Compound |
| Chlormequat chloride | 999-81-5 | 18101 | 1512 | Plant Growth | Quaternary |
| | | | | Regulator | Ammonium |
| | | | | | Compound |
| Di(alkyl* oxypropyl)dimethyl | NA 1261 | 69121 | NA 173 | Algaecide, | Quaternary |
| ammonium chloride *(60% C8, | | | | Microbiocide | Ammonium |
| 40% C10) | | | | | Compound |
| Dialkyl (85% C18, 15% C16) | 68514-95-4 | NA 355 | 197 | Microbiocide, | Quaternary |
| dimethyl ammonium chloride | | | | Adjuvant | Ammonium |
| | | | | | Compound |
| Dialkyl dimethyl ammonium | NA 900 | NA 172 | 5181 | Microbiocide | Quaternary |
| polynaphthyl amine | | | | | Ammonium |
| | | | | | Compound |
| Dialkyl* dimethyl ammonium | 73398-64-8 | 69136 | 2230 | Microbiocide | Quaternary |
| chloride *(47% C12, 18% C14, | | | | | Ammonium |
| 10% C18, 9% C10, 8% C16, | | | | | Compound |
| 8% C8) | | | | | |
| Dialkyl* dimethyl ammonium | 73398-64-8 | ##### | NA 114 | Microbiocide | Quaternary |
| chloride *(48% C12, 17% C14, | | | | | Ammonium |
| 10% C18, 9% C16, 8% C8, 7% | | | | | Compound |
| C10, 1% C6) | | | | | |
| Dialkyl* dimethyl ammonium | 68153-33-3 | ##### | NA 113 | Microbiocide | Quaternary |
| chloride *(50% C12, 20% C14, | | | | | Ammonium |
| 15% C10, 10% C16, 5% C18) | | | | | Compound |
| Dialkyl* dimethyl ammonium | 68910-56-5 | 69177 | NA 645 | Microbiocide | Quaternary |
| chloride *(50% C12, 30% C14, | | | | | Ammonium |
| 20% C16) | | | | | Compound |
| Dialkyl* dimethyl ammonium | 68002-59-5 | 69120 | NA 626 | Microbiocide | Quaternary |
| chloride *(70% C18, 26% C16, | | | | | Ammonium |
| 4% C14) | | | | | Compound |
| Dialkyl* methyl benzyl | 73049-75-9 | 69119 | NA 625 | Algaecide, | Quaternary |
| ammonium chloride *(60% | | | | Microbiocide | Ammonium |
| C14, 30% C16, 5% C18, 5% | | | | | Compound |
| C12) | | | | | |
| Diallyl dimethyl ammonium | 26062-79-3 | ##### | 5092 | Adjuvant | Quaternary |
| chloride polymers | | | | | Ammonium |
| | | | | | Compound |
| Dibenzyldimethylammonium | NA 1440 | NA 213 | NA 213 | Microbiocide | Quaternary |
| chloride | | | | | Ammonium |
| | | | | | Compound |
| Dicoco dimonium chloride | 61789-77-3 | 69138 | 3140 | Adjuvant | Quaternary |
| | | | | | Ammonium |
| | | | | | Compound |
| Didecyl dimethyl ammonium | 148788-55-0 | 69208 | NA 658 | Microbiocide | Quaternary |
| carbonate and didecyl dimethyl | | | | | Ammonium |
| ammonium bicarbonate | | | | | Compound |
| Didecyl dimethyl ammonium | 148812-65-1 | 69208 | NA 659 | Microbiocide | Quaternary |
| carbonate and didecyl dimethyl | | | | | Ammonium |
| ammonium bicarbonate | | | | | Compound |
| Didecyl dimethyl ammonium | 7173-51-5 | 69149 | 1682 | Microbiocide, | Quaternary |
| chloride | | | | Fungicide | Ammonium |
| | | | | | Compound |
| Didecyl methyl benzyl | 32426-10-1 | 69170 | NA 642 | Microbiocide | Quaternary |
| ammonium chloride | | | | | Ammonium |
| | | | | | Compound |
| Dihydrogenated tallow | 61789-81-9 | 69150 | 3153 | Adjuvant | Quaternary |
| dimethyl ammonium | | | | | Ammonium |
| methosulfate | | | | | Compound |
| Dilauryl dimethyl ammonium | 3282-73-3 | ##### | NA 112 | Microbiocide | Quaternary |
| bromide | | | | | Ammonium |
| | | | | | Compound |
| Dimethyl dicoco ammonium | NA 166 | NA 317 | 1945 | Microbiocide | Quaternary |
| chloride | | | | | Ammonium |
| | | | | | Compound |
| Dimethyl dioctadecyl | NA 627 | NA 114 | 3163 | Adjuvant | Quaternary |
| ammonium bentonite | | | | | Ammonium |
| | | | | | Compound |
| Dioctyl dimethyl ammonium | 5538-94-3 | 69166 | 1710 | Microbiocide, | Quaternary |
| chloride | | | | Algaecide, | Ammonium |
| | | | | Fungicide | Compound |
| Distearyl dimonium chloride | 107-64-2 | ##### | 3176 | Adjuvant | Quaternary |
| | | | | | Ammonium |
| | | | | | Compound |
| Dodecyl di(2-hydroxyethyl) | 19379-90-9 | 69181 | NA 647 | Microbiocide | Quaternary |
| benzyl ammonium chloride | | | | | Ammonium |
| | | | | | Compound |
| Dodecyl dimethyl 2,4,5- | 53404-46-9 | ##### | NA 113 | Microbiocide | Quaternary |
| trimethylbenzyl ammonium | | | | | Ammonium |
| chloride | | | | | Compound |
| Dodecyl dimethyl ammonium | NA 157 | NA 294 | 1008 | Microbiocide | Quaternary |
| chloride | | | | | Ammonium |
| | | | | | Compound |
| Dodecyl dimethyl benzyl | 1964001 | 69123 | NA 627 | Fungicide, | Quaternary |
| ammonium bromide | | | | Microbiocide | Ammonium |
| | | | | | Compound |
| Dodecyl dimethyl benzyl | 139-07-1 | 69124 | 1167 | Microbiocide, | Quaternary |
| ammonium chloride | | | | Herbicide | Ammonium |
| | | | | | Compound |
| Dodecyl dimethyl benzyl | 22232-26-4 | 69128 | NA 629 | Microbiocide | Quaternary |
| ammonium | | | | | Ammonium |
| cyclopentanecarboxylate | | | | | Compound |
| Dodecyl dimethyl benzyl | NA 1027 | 69127 | 1446 | Fungicide, | Quaternary |
| ammonium naphthenate | | | | Insecticide, | Ammonium |
| | | | | Fungicide | Compound |
| Dodecyl dimethyl | 53466-90-3 | ##### | NA 113 | Microbiocide | Quaternary |
| trichlorobenzyl ammonium | | | | | Ammonium |
| chloride | | | | | Compound |
| Dodecylbenzyl alkyl (70% C12, | 87175-02-8 | ##### | 1038 | Algaecide, | Quaternary |
| 30% C14) dimethyl ammonium | | | | Microbiocide | Ammonium |
| chloride | | | | | Compound |
| Dodecylbenzyl octadecyl | 29932-85-2 | ##### | NA 113 | Microbiocide | Quaternary |
| dimethyl ammonium chloride | | | | | Ammonium |
| | | | | | Compound |
| Dodecylbenzyl trimethyl | 12379-51-0 | 69126 | NA 628 | N/A | Quaternary |
| ammonium 2-ethylhexanoate | | | | | Ammonium |
| | | | | | Compound |
| Dodecylbenzyl trimethyl | 1330-85-4 | 69125 | 918 | Adjuvant, | Quaternary |
| ammonium chloride | | | | Microbiocide | Ammonium |
| | | | | | Compound |
| Ecolyst | NA 1174 | 69089 | NA 165 | Plant Growth | Quaternary |
| | | | | Regulator | Ammonium |
| | | | | | Compound |
| Ethyl dimethyl stearyl | 111-98-8 | ##### | NA 115 | Microbiocide | Quaternary |
| ammonium bromide | | | | | Ammonium |
| | | | | | Compound |
| Mepiquat | 15302-91-7 | NA 203 | NA 202 | Plant Growth | Quaternary |
| | | | | Regulator | Ammonium |
| | | | | | Compound |
| Mepiquat chloride | 24307-26-4 | ##### | 2075 | Plant Growth | Quaternary |
| | | | | Regulator | Ammonium |
| | | | | | Compound |
| Methyl dodecyl benzyl | 1399-80-0 | 69129 | 895 | Microbiocide | Quaternary |
| trimethyl ammonium chloride | | | | | Ammonium |
| | | | | | Compound |
| Methyl dodecyl benzyl | NA 619 | NA 112 | 2678 | Microbiocide | Quaternary |
| trimethyl ammonium chloride | | | | | Ammonium |
| 80% and methyl | | | | | Compound |
| Methyl dodecyl trimethyl | NA 44 | NA 83 | 2679 | Microbiocide | Quaternary |
| ammonium chloride | | | | | Ammonium |
| | | | | | Compound |
| Methyl dodecyl xylene bis | NA 45 | NA 84 | 896 | Microbiocide | Quaternary |
| (trimethyl ammonium chloride) | | | | | Ammonium |
| | | | | | Compound |
| Methyl trioctyl ammonium | 5137-55-3 | NA 72 | 3291 | Adjuvant | Quaternary |
| chloride | | | | | Ammonium |
| | | | | | Compound |
| Morpholine dodecyl benzene | 12068-08-5 | ##### | 3294 | Microbiocide, | Quaternary |
| sulfonate | | | | Adjuvant, | Ammonium |
| | | | | Fungicide, | Compound |
| | | | | Insecticide | |
| N-(3,4-dichlorobenzyl)-N- | 102-30-7 | ##### | 1129 | Microbiocide | Quaternary |
| dodecyl-N,N-dimethyl | | | | | Ammonium |
| ammonium chloride | | | | | Compound |
| N-alkyl (92% C18, 8% C16)-N- | 61791-34-2 | 69113 | 986 | Fungicide, | Quaternary |
| ethyl morpholinium ethyl | | | | Microbiocide | Ammonium |
| sulfate | | | | | Compound |
| N-alkyl (soza)-N-methyl | NA 281 | NA 550 | 3020 | Fungicide, | Quaternary |
| morpholinium sulfate | | | | Microbiocide | Ammonium |
| | | | | | Compound |
| N-cetyl-N-ethyl morpholinium | 78-21-7 | 69113 | 1792 | Fungicide, | Quaternary |
| ethyl sulfate | | | | Microbiocide | Ammonium |
| | | | | | Compound |
| N-Cetyl-N-ethyl morpholinium | 78-21-7 | ##### | NA 174 | Fungicide | Quaternary |
| ethyl sulfate | | | | | Ammonium |
| | | | | | Compound |
| N-Cetyl-N-ethylmorpholinium | 78-21-7 | 69187 | NA 172 | Fungicide | Quaternary |
| ethyl sulfate | | | | | Ammonium |
| | | | | | Compound |
| N-Dialkyl (60% C14, 30% C16, | 68424-87-3 | NA 356 | 2070 | Microbiocide | Quaternary |
| 5% C12, 5% C18) methyl | | | | | Ammonium |
| benzyl ammonium chloride | | | | | Compound |
| N-isononyl-N,N-dimethyl | 138698-36-9 | 69207 | 5070 | Microbiocide | Quaternary |
| decanaminium chloride | | | | | Ammonium |
| | | | | | Compound |
| Octyl decyl dimethyl | 32426-11-2 | 69165 | 1709 | Microbiocide, | Quaternary |
| ammonium chloride | | | | Fungicide | Ammonium |
| | | | | | Compound |
| Octyl dodecyl dimethyl | 10361-16-7 | 69190 | 1947 | Microbiocide | Quaternary |
| ammonium chloride | | | | | Ammonium |
| | | | | | Compound |
| Oleyl dimethyl ethyl | 14351-44-1 | 69132 | NA 631 | Microbiocide | Quaternary |
| ammonium bromide | | | | | Ammonium |
| | | | | | Compound |
| Oxydiethylene bis (alkyl | 68607-28-3 | 69173 | 1362 | Microbiocide | Quaternary |
| dimethyl ammonium chloride), | | | | | Ammonium |
| alkyl derived from coconut oil | | | | | Compound |
| fatty acids | | | | | |
| Polybromide form of | NA 1164 | 8711 | NA 163 | Microbiocide | Quaternary |
| trimethylbenzyl ammonium | | | | | Ammonium |
| resin | | | | | Compound |
| Polyethoxypolypropoxypolyethoxyethanol- | NA 1287 | 46913 | NA 175 | Microbiocide | Iodine Compound, |
| n-alkyl (54% C12, | | | | | Quaternary |
| 18% C14, 9% C16, 9% C18, | | | | | Ammonium |
| 5% C10, 5% C8) di(beta- | | | | | Compound |
| hydroxyethyl) benzyl am | | | | | |
| Polymeric quaternary | NA 1463 | NA 217 | NA 216 | Microbiocide, | Quaternary |
| ammonium chloride | | | | Adjuvant | Ammonium |
| | | | | | Compound |
| Polyquaternium-6 | 28301-34-0 | NA 767 | 2818 | Adjuvant | Quaternary |
| | | | | | Ammonium |
| | | | | | Compound |
| Polytrimethyl vinyl benzyl | 9017-80-5 | NA 119 | 3769 | Adjuvant | Quaternary |
| ammonium chloride | | | | | Ammonium |
| | | | | | Compound |
| Quaternium-18 bentonite | 68953-58-2 | ##### | 2996 | Adjuvant | Quaternary |
| | | | | | Ammonium |
| | | | | | Compound |
| Soya ethyl morpholinium | 61791-34-2 | 69113 | 3453 | Fungicide, | Quaternary |
| ethosulfate | | | | Microbiocide | Ammonium |
| | | | | | Compound |
| Tetradecyl dimethyl benzyl | 139-08-2 | 69107 | 1166 | Algaecide, | Quaternary |
| ammonium chloride | | | | Microbiocide | Ammonium |
| | | | | | Compound |
| Tetraethyl ammonium | 77-98-5 | 4215 | NA 76 | N/A | Quaternary |
| hydroxide | | | | | Ammonium |
| | | | | | Compound |
| 2-Mercaptobenzothiazole, zinc | 155-04-4 | 51705 | 1449 | Fungicide, | Mercaptobenzothiazole, |
| salt | | | | Microbiocide | Inorganic-Zinc |
| 5-Chloro-2- | 53404-93-6 | 51709 | NA 427 | Microbiocide, | Mercaptobenzothiazole, |
| mercaptobenzothiazole, zinc | | | | Fungicide | Inorganic-Zinc |
| salt | | | | | |
| Acetic acid, copper (2+) salt | 142-71-2 | NA 697 | 3013 | N/A | Inorganic-Copper |
| Alkyl*-1-(2-aminoethyl)-2- | NA 1188 | 46606 | NA 167 | Microbiocide | Inorganic-Nickel, |
| imidazoline acetate-nickel | | | | | Imidazoline |
| sulfate complex | | | | | |
| Aluminum | 7429-90-5 | 111 | 1042 | N/A | Inorganic |
| Aluminum acetate | NA 971 | NA 548 | 3025 | N/A | Inorganic |
| Aluminum bicarbonate | NA 1043 | NA 140 | 3026 | N/A | Inorganic |
| Aluminum caprylate | 6028-57-5 | NA 549 | 3027 | N/A | Inorganic |
| Aluminum chloride | 7446-70-0 | 13901 | 3028 | N/A | Inorganic |
| Aluminum diacetate | 142-03-0 | NA 113 | 3029 | N/A | Inorganic |
| Aluminum hydroxide | 21645-51-2 | NA 129 | 2145 | N/A | Inorganic |
| Aluminum magnesium silicate | NA 1018 | NA 107 | 2378 | Adjuvant | Inorganic |
| Aluminum oxide | 1344-28-1 | NA 540 | 3030 | N/A | Inorganic |
| Aluminum phosphide | 20859-73-8 | 66501 | 484 | Fumigant, | Inorganic |
| | | | | Fungicide, | |
| | | | | Fungicide | |
| Aluminum potassium sulfate | 10043-67-1 | ##### | 3031 | Herbicide, | Inorganic |
| | | | | Insecticide | |
| Aluminum silicate | 1302-76-7 | NA 131 | 2379 | Adjuvant | Inorganic |
| Aluminum stearate | 637-12-7 | NA 541 | 3032 | N/A | Inorganic |
| Aluminum sulfate | 10043-01-3 | 13906 | 1415 | Plant Growth | Inorganic |
| | | | | Regulator, | |
| | | | | Molluscicide | |
| Ammonia | 7664-41-7 | 5302 | 22 | Insecticide, | Inorganic |
| | | | | Deer Repellent, | |
| | | | | Fungicide | |
| Ammonium alum | 7784-26-1 | NA 533 | 3037 | Repellent | Inorganic |
| Ammonium alum | NA 1216 | 98501 | NA 170 | Adjuvant | Inorganic |
| Ammonium arsenate | 7784-44-3 | 13601 | 2380 | Herbicide, | Inorganic-arsenic, |
| | | | | Insecticide, | Heavy metal |
| | | | | Rodenticide | |
| Ammonium bicarbonate | 1066-33-7 | NA 534 | 3038 | pH Adjustment | Inorganic |
| Ammonium bisulfate | 7803-63-6 | NA 141 | 3039 | N/A | Inorganic |
| Ammonium bromide | 12124-97-9 | 352 | 3040 | N/A | Inorganic |
| Ammonium carbamate | 1111-89-7 | NA 141 | 3041 | N/A | Inorganic |
| Ammonium carbonate | 506-87-6 | 73501 | 3042 | Fungicide, | Inorganic |
| | | | | Herbicide, | |
| | | | | Microbiocide, | |
| | | | | pH Adjustment | |
| Ammonium chloride | 12125-02-9 | ##### | 3043 | N/A | Inorganic |
| Ammonium dichromate | 2149701 | 68305 | NA 607 | Wood | Inorganic- |
| | | | | Preservative | Chromium(VI), |
| | | | | | Heavy metal |
| Ammonium ferric sulfate | 10138-04-2 | NA 537 | 3047 | Herbicide | Inorganic |
| Ammonium fluosilicate | 16919-19-0 | 75301 | 695 | Insecticide | Inorganic |
| Ammonium fluosilicate on | 62449-69-8 | 75311 | NA 694 | Insecticide | Inorganic |
| silica gel | | | | | |
| Ammonium hydroxide | 1336-21-6 | 5301 | 2381 | Fungicide, pH | Inorganic |
| | | | | Adjustment, | |
| | | | | Herbicide, | |
| | | | | Microbiocide | |
| Ammonium nitrate | 6484-52-2 | 76101 | 3052 | Rodenticide, | Inorganic |
| | | | | Microbiocide | |
| Ammonium nitrite | 13446-48-5 | 76201 | NA 700 | Rodenticide, | Inorganic |
| | | | | Microbiocide | |
| Ammonium oxalate | 1113-38-8 | 9603 | 891 | Microbiocide | Inorganic |
| Ammonium polysulfides | 9080-17-5 | 76701 | NA 707 | Insecticide, | Inorganic |
| | | | | Fungicide | |
| Ammonium sulfate | 7783-20-2 | 5601 | 1363 | Herbicide, pH | Inorganic |
| | | | | Adjustment | |
| Ammonium thiocyanate | 1762-95-4 | 68200 | 25 | Synergist | Inorganic |
| Ammonium thiosulfate | 7783-18-8 | 80103 | 892 | Insecticide, | Inorganic |
| | | | | Fungicide, | |
| | | | | Herbicide | |
| Anhydrous zirconium (IV) | 1314-23-4 | 79090 | 2204 | N/A | Inorganic |
| oxide | | | | | |
| Anilinocadmium dilactate | 19651-91-3 | 64601 | NA 548 | N/A | Inorganic-Cadmium, |
| | | | | | Heavy metal |
| Arsenic | 7440-38-2 | NA 121 | 710 | Herbicide, | Inorganic-arsenic, |
| | | | | Insecticide, | Heavy metal |
| | | | | Rodenticide | |
| Arsenic acid | 7778-39-4 | 6801 | 40 | Herbicide, | Inorganic-arsenic, |
| | | | | Insecticide, | Heavy metal |
| | | | | Rodenticide | |
| Arsenic disulfide | 56320-22-0 | 6901 | NA 98 | Herbicide, | Inorganic-arsenic, |
| | | | | Insecticide, | Heavy metal |
| | | | | Rodenticide | |
| Arsenic pentoxide | 1303-28-2 | 6802 | 631 | Fungicide, | Inorganic-arsenic, |
| | | | | Insecticide, | Heavy metal |
| | | | | Rodenticide, | |
| | | | | Herbicide | |
| Arsenic sulfide | 12612-21-4 | 6901 | NA 99 | Herbicide, | Inorganic-arsenic, |
| | | | | Insecticide, | Heavy metal |
| | | | | Rodenticide | |
| Arsenic trioxide | 1327-53-3 | 7001 | 42 | Herbicide, | Inorganic-arsenic, |
| | | | | Insecticide, | Heavy metal |
| | | | | Rodenticide | |
| Arsenic trisulfide | 1303-33-9 | 6901 | NA 97 | Herbicide, | Inorganic-arsenic, |
| | | | | Insecticide, | Heavy metal |
| | | | | Rodenticide | |
| Arsonic acid, ammonium salt | 58829-95-1 | ##### | NA 105 | Herbicide, | Inorganic-arsenic, |
| | | | | Insecticide, | Heavy metal |
| | | | | Wood | |
| | | | | Preservative | |
| Asbestos | 1332-21-4 | 99301 | 2398 | Insecticide | Inorganic |
| Barium carbonate | 513-77-9 | 7501 | 3060 | N/A | Inorganic |
| Barium chloride | 10361-37-2 | NA 141 | 3061 | N/A | Inorganic |
| Barium fluosilicate | 17125-80-3 | 75302 | NA 688 | Insecticide | Inorganic |
| Barium metaborate | 13701-59-2 | 11101 | 973 | Fungicide | Inorganic |
| Barium nitrate | 10022-31-8 | NA 186 | NA 185 | Repellent | Inorganic |
| Barium polysulfide | 50864-67-0 | 76704 | NA 709 | Insecticide, | Inorganic |
| | | | | Fungicide | |
| Barium sulfate | 7727-43-7 | 7502 | 2411 | N/A | Inorganic |
| Basic copper arsenate | 16102-92-4 | ##### | NA 132 | Herbicide, | Inorganic-arsenic, |
| | | | | Insecticide, | Heavy metal, |
| | | | | Rodenticide | Inorganic-Copper |
| Basic lead silicate | 53466-66-3 | 48003 | NA 417 | N/A | Inorganic-Lead, |
| | | | | | Heavy metal |
| Bentonite | 1302-78-9 | NA 496 | 1399 | Adjuvant | Inorganic |
| Blast furnace slag | NA 239 | NA 477 | 2425 | N/A | Inorganic |
| Borax | 1303-96-4 | 11102 | 79 | Insecticide, | Inorganic |
| | | | | Herbicide | |
| Bordeaux mixture | NA 240 | NA 478 | 80 | Fungicide | Inorganic-Copper |
| Boric acid | 10043-35-3 | 11001 | 769 | Insecticide | Inorganic |
| Boric acid(H4B6O11), zinc | 12447-61-9 | ##### | 5094 | Fungicide | Inorganic-Zinc |
| salt(1:2) | | | | | |
| Boric oxide | 1303-86-2 | 11002 | 2090 | Insecticide | Inorganic |
| Boron | 7440-42-8 | ##### | 2426 | N/A | Inorganic |
| Boron sodium oxide | 12179-04-3 | 11110 | NA 116 | Insecticide | Inorganic |
| (B4Na2O7), pentahydrate | | | | | |
| Bromine | 7726-95-6 | 8701 | 2296 | Microbiocide | Inorganic |
| Bromine chloride | 13863-41-7 | 20504 | 2323 | Microbiocide | Inorganic |
| Cadmium | 7440-43-9 | NA 460 | 2438 | N/A | Inorganic-Cadmium, |
| | | | | | Heavy metal |
| Cadmium carbonate | 513-78-0 | 12901 | 93 | N/A | Inorganic-Cadmium, |
| | | | | | Heavy metal |
| Cadmium chloride | 10108-64-2 | 12902 | 94 | N/A | Inorganic-Cadmium, |
| | | | | | Heavy metal |
| Cadmium cocoate | 72869-63-7 | NA 459 | 3085 | N/A | Inorganic-Cadmium, |
| | | | | | Heavy metal |
| Cadmium oxide | 1306-19-0 | ##### | NA 130 | N/A | Inorganic-Cadmium, |
| | | | | | Heavy metal |
| Cadmium perborate | NA 969 | NA 461 | 3086 | N/A | Inorganic-Cadmium, |
| | | | | | Heavy metal |
| Cadmium sebacate | 939499 | 12903 | 699 | N/A | Inorganic-Cadmium, |
| | | | | | Heavy metal |
| Cadmium succinate | 141-00-4 | 12904 | 92 | N/A | Inorganic-Cadmium, |
| | | | | | Heavy metal |
| Cadmium sulfate | 10124-36-4 | 12905 | NA 123 | N/A | Inorganic-Cadmium, |
| | | | | | Heavy metal |
| Cadmium yellow pigments | NA 967 | NA 448 | 2439 | N/A | Inorganic-Cadmium, |
| | | | | | Heavy metal |
| Calcareous shale | NA 230 | NA 449 | 3595 | N/A | Inorganic |
| Calcium arsenate | 7778-44-1 | 13501 | 96 | Herbicide, | Inorganic-arsenic, |
| | | | | Insecticide, | Heavy metal |
| | | | | Rodenticide, | |
| | | | | Molluscicide | |
| Calcium arsenite | 53404-59-4 | 13602 | NA 129 | Herbicide, | Inorganic-arsenic, |
| | | | | Insecticide, | Heavy metal |
| | | | | Rodenticide | |
| Calcium carbide | 75-20-7 | NA 450 | 1571 | Repellent | Inorganic |
| Calcium carbonate | 471-34-1 | 73502 | 1007 | pH Adjustment, | Inorganic |
| | | | | Fungicide, | |
| | | | | Microbiocide, | |
| | | | | Herbicide | |
| Calcium carbonate, treated | NA 968 | NA 451 | 2440 | pH Adjustment, | Inorganic |
| | | | | Fungicide, | |
| | | | | Microbiocide, | |
| | | | | Herbicide | |
| Calcium chlorate | 10137-74-3 | 73302 | NA 682 | Defoliant, | Inorganic |
| | | | | Herbicide, | |
| | | | | Microbiocide | |
| Calcium chloride | 10043-52-4 | 75605 | 920 | Fungicide, | Inorganic |
| | | | | Herbicide, | |
| | | | | Insecticide, | |
| | | | | Microbiocide, | |
| | | | | Molluscicide, | |
| | | | | Dessicant, Plant | |
| | | | | Growth | |
| | | | | Regulator | |
| Calcium cyanide | 592-01-8 | 74001 | 176 | Rodenticide | Inorganic |
| Calcium hydroxide | 1305-62-0 | 75601 | 99 | pH Adjustment, | Inorganic |
| | | | | Fungicide, | |
| | | | | Microbiocide, | |
| | | | | Herbicide | |
| Calcium hypochlorite | 7778-54-3 | 14701 | 326 | Algaecide, | Inorganic |
| | | | | Water | |
| | | | | Treatment | |
| Calcium nitrite | 13780-06-8 | 76202 | NA 701 | Rodenticide, | Inorganic |
| | | | | Microbiocide | |
| Calcium phosphate | 10103-46-5 | NA 195 | NA 195 | Rodenticide | Inorganic |
| Calcium phosphate, tribasic | 7758-87-4 | 76401 | 1607 | Rodenticide, | Inorganic |
| | | | | Fungicide, | |
| | | | | Herbicide, | |
| | | | | Microbiocide | |
| Calcium silicate | 10101-39-0 | 72607 | 1908 | Insecticide, | Inorganic |
| | | | | Adjuvant, | |
| | | | | Fungicide, | |
| | | | | Microbiocide | |
| Calcium sulfate | 7778-18-9 | 5602 | 2442 | Insecticide, | Inorganic |
| | | | | Herbicide | |
| Calcium sulfate dihydrate | 10101-41-4 | 203 | NA 163 | Insecticide, | Inorganic |
| | | | | Herbicide | |
| Calcium thiosulfate | 10124-41-1 | 80101 | 930 | Insecticide, | Inorganic |
| | | | | Fungicide, | |
| | | | | Herbicide | |
| Calcium-zinc LN 193 | NA 653 | NA 120 | 3871 | N/A | Inorganic-Zinc |
| (complex) | | | | | |
| Calomel | 10112-91-1 | 52201 | 100 | Fungicide | Inorganic-Mercury, |
| | | | | | Heavy metal |
| Carbon | 7440-44-0 | 16001 | 712 | Water | Inorganic |
| | | | | Treatment, | |
| | | | | Rodenticide | |
| Carbon black pigment | 1333-86-4 | NA 442 | 2445 | Dye | Inorganic |
| Carbon dioxide | 124-38-9 | 16601 | 2207 | Fumigant, | Inorganic |
| | | | | Insecticide, | |
| | | | | Rodenticide | |
| Carbon disulfide | 75-15-0 | 16401 | 108 | Fumigant, | Inorganic |
| | | | | Solvent, | |
| | | | | Breakdown | |
| | | | | product, | |
| | | | | Nematicide | |
| Chlorinated trisodium | 56802-99-4 | ##### | 2214 | Water | Inorganic |
| phosphate (prior TSP & | | | | Treatment | |
| sodium hypochlorite) | | | | | |
| Chlorine | 7782-50-5 | 20501 | 131 | Microbiocide, | Inorganic |
| | | | | Water | |
| | | | | Treatment | |
| Chlorine dioxide | 10049-04-4 | 20503 | 2053 | Microbiocide, | Inorganic |
| | | | | Water | |
| | | | | Treatment | |
| Chlorophyllin | 11006-34-1 | NA 191 | NA 190 | Fungicide, | Inorganic-Copper, |
| | | | | Microbiocide | Botanical |
| Chromated zinc chloride | NA 1026 | 87801 | 1187 | Insecticide, | Inorganic- |
| | | | | Herbicide, | Chromium(VI), |
| | | | | Microbiocide | Inorganic-Zinc, |
| | | | | | Heavy metal |
| Chromic acetate | 1066-30-4 | 21102 | NA 172 | Wood | Inorganic- |
| | | | | Preservative | Chromium(VI), |
| | | | | | Heavy metal |
| Chromic acid | 7738-94-5 | 21101 | 1188 | Wood | Inorganic- |
| | | | | Preservative | Chromium(VI), |
| | | | | | Heavy metal |
| Chromic oxide | 1308-38-9 | 21103 | NA 173 | Wood | Inorganic- |
| | | | | Preservative | Chromium(VI), |
| | | | | | Heavy metal |
| Chromium dioxide | 12018-01-8 | NA 411 | 2470 | N/A | Inorganic |
| Cobaltous sulfate | 10124-43-3 | NA 114 | 3110 | N/A | Inorganic |
| Copper | 7440-50-8 | 22501 | 714 | Fungicide | Inorganic-Copper |
| Copper (from triethanolamine | 82027-59-6 | 24403 | NA 198 | Algaecide | Inorganic-Copper |
| complex) | | | | | |
| Copper 2-ethylhexanoate | 22221-10-9 | 41201 | 2235 | Fungicide | Inorganic-Copper |
| Copper 3-phenylsalicylate | 1250682 | 23801 | NA 191 | N/A | Inorganic-Copper |
| Copper 8-quinolinoleate | 10380-28-6 | 24002 | 159 | Fungicide, | Inorganic-Copper |
| | | | | Microbiocide | |
| Copper abietate | 10248-55-2 | 23301 | NA 184 | Fungicide | Inorganic-Copper |
| Copper acetate | 831632 | 44002 | 147 | Fungicide | Inorganic-Copper |
| Copper ammonium acetate | NA 1544 | NA 225 | NA 225 | Fungicide | Inorganic-Copper |
| Copper ammonium carbonate | 33113-08-5 | 22703 | 1762 | Fungicide | Inorganic-Copper |
| Copper ammonium complex | 16828-95-8 | 22702 | 3550 | Fungicide | Inorganic-Copper |
| Copper arsenate | 10103-61-4 | 22801 | NA 178 | Herbicide, | Inorganic-arsenic, |
| | | | | Insecticide, | Heavy metal, |
| | | | | Rodenticide | Inorganic-Copper |
| Copper arsenite | 10290-12-7 | 22401 | NA 177 | Herbicide, | Inorganic-arsenic, |
| | | | | Insecticide, | Heavy metal, |
| | | | | Rodenticide | Inorganic-Copper |
| Copper as elemental from | 50376-91-5 | 24404 | NA 199 | N/A | Inorganic-Copper |
| copper - etidronic acid complex | | | | | |
| Copper beta cyclodextrin | NA 1366 | NA 193 | NA 193 | Fungicide | Inorganic-Copper |
| hydroxide | | | | | |
| Copper bronze powder | 7440-50-8 | 22501 | 1457 | Antifoulant | Inorganic-Copper |
| Copper carbonate, basic | 1184-64-1 | 22901 | 60 | Fungicide, | Inorganic-Copper |
| | | | | Algaecide, | |
| | | | | Insecticide | |
| Copper chloride (anhydrous) | 1344-67-8 | 23802 | NA 192 | N/A | Inorganic-Copper |
| Copper chloride (dihydrate) | 10125-13-0 | 23701 | NA 190 | N/A | Inorganic-Copper |
| Copper citrate | 10402-15-0 | 44005 | 1406 | N/A | Inorganic-Copper |
| Copper citrate chelate | NA 961 | NA 387 | 3547 | N/A | Inorganic-Copper |
| Copper complex with ammonia | 67989-88-2 | ##### | NA 130 | Fungicide | Inorganic-Copper |
| and ethylene diamine | | | | | |
| tetraacetate | | | | | |
| Copper cresylate | 12379-42-9 | 23001 | NA 181 | Fungicide | Inorganic-Copper |
| Copper | 53404-24-3 | 41202 | NA 349 | N/A | Inorganic-Copper |
| dehydroabietylammonium 2- | | | | | |
| ethylhexanoate | | | | | |
| Copper dihydrazinium sulfate | 33271-65-7 | 50504 | 753 | N/A | Inorganic-Copper |
| Copper ethanolamine complex | 14215-52-2 | 24409 | NA 201 | Algaecide | Inorganic-Copper |
| Copper ethanolamine | NA 1044 | NA 146 | 3551 | Algaecide | Inorganic-Copper |
| complexes, mixed | | | | | |
| Copper ethylenediamine | 13426-91-0 | 24407 | 3549 | Herbicide | Inorganic-Copper |
| complex | | | | | |
| Copper gluconate chelate | 814-91-5 | 23305 | 3548 | N/A | Inorganic-Copper |
| Copper hydrazinium sulfate | 53433-02-6 | 50501 | NA 418 | Fungicide | Inorganic-Copper |
| Copper hydroxide | 20427-59-2 | 23401 | 151 | Fungicide, | Inorganic-Copper |
| | | | | Microbiocide, | |
| | | | | Nematicide | |
| Copper hydroxide - | NA 1017 | NA 105 | 1826 | N/A | Inorganic-Copper |
| triethanolamine complex | | | | | |
| Copper isodecanoate | 84082-88-2 | 41221 | NA 354 | N/A | Inorganic-Copper |
| Copper isononanoate | 72915-82-3 | 41211 | NA 352 | N/A | Inorganic-Copper |
| Copper isooctanoate | 88859-94-3 | 41204 | NA 350 | N/A | Inorganic-Copper |
| Copper lactate | 16039-52-4 | 23302 | NA 185 | N/A | Inorganic-Copper |
| Copper linoleate | 7721-15-5 | 23303 | 1110 | N/A | Inorganic-Copper |
| Copper metaborate | 39290-85-2 | 22802 | NA 179 | N/A | Inorganic-Copper |
| Copper monoethanolamine | NA 960 | NA 384 | 3552 | N/A | Inorganic-Copper |
| complex | | | | | |
| Copper naphthenate | 1338-02-9 | 23102 | 153 | Wood | Inorganic-Copper |
| | | | | Preservative, | |
| | | | | Insecticide, | |
| | | | | Fungicide, Dog | |
| | | | | and Cat | |
| | | | | Repellent | |
| Copper naphthenate | 1338-02-9 | 6000 | NA 172 | Wood | Inorganic-Copper |
| | | | | Preservative, | |
| | | | | Insecticide, | |
| | | | | Fungicide, Dog | |
| | | | | and Cat | |
| | | | | Repellent | |
| Copper naphthenate | 1338-02-9 | 6300 | NA 172 | Wood | Inorganic-Copper |
| | | | | Preservative, | |
| | | | | Insecticide, | |
| | | | | Fungicide, Dog | |
| | | | | and Cat | |
| | | | | Repellent | |
| Copper nitroacetate | 22221-12-1 | 23201 | NA 183 | N/A | Inorganic-Copper |
| Copper octanoate | 20543-04-8 | 23306 | 5225 | Fungicide | Inorganic-Copper |
| Copper oleate | 10402-16-1 | 23304 | 154 | Fungicide | Inorganic-Copper |
| Copper oxide (ic) | 1317-38-0 | 42401 | 2231 | Fungicide, | Inorganic-Copper |
| | | | | Insecticide | |
| Copper oxide (ous) | 1317-39-1 | 25601 | 175 | Fungicide, | Inorganic-Copper |
| | | | | Insecticide | |
| Copper oxychloride | 1332-40-7 | 8001 | 156 | Fungicide | Inorganic-Copper |
| Copper oxychloride | 1332-65-6 | 23501 | NA 186 | Fungicide | Inorganic-Copper |
| (Cu2Cl(OH)3) | | | | | |
| Copper oxychloride sulfate | 8012-69-9 | 23503 | 158 | Fungicide | Inorganic-Copper |
| Copper oxysulfate | 12158-97-3 | 23504 | NA 188 | N/A | Inorganic-Copper |
| Copper oxysulfate | 82010-79-5 | 23504 | NA 189 | N/A | Inorganic-Copper |
| Copper pentachlorophenate | 15773-35-0 | 63011 | NA 512 | Wood | Chlorinated Phenol, |
| | | | | Preservative, | Inorganic-Copper |
| | | | | Microbiocide, | |
| | | | | Algaecide, | |
| | | | | Fungicide | |
| Copper phosphate | 10103-48-7 | 22902 | NA 180 | N/A | Inorganic-Copper |
| Copper phthalocyanine | 147-14-8 | NA 381 | 2479 | Dye | Inorganic-Copper |
| Copper pyrophosphate | 10102-90-6 | 69701 | NA 663 | N/A | Inorganic-Copper |
| Copper salts of fatty and rosin | 9007-39-0 | 23104 | 155 | Fungicide | Inorganic-Copper |
| acids | | | | | |
| Copper salts of the acids of tall | 61789-22-8 | 23103 | NA 182 | N/A | Inorganic-Copper |
| oil | | | | | |
| Copper silicate | 1344-72-5 | 24301 | NA 197 | N/A | Inorganic-Copper |
| Copper sodium sulfate- | NA 1023 | NA 121 | 755 | N/A | Inorganic-Copper |
| phosphate complex | | | | | |
| Copper sulfate (anhydrous) | 7758-98-7 | 24408 | 1778 | Fungicide, | Inorganic-Copper |
| | | | | Algaecide, | |
| | | | | Molluscicide | |
| Copper sulfate (basic) | 1344-73-6 | 8101 | 162 | Fungicide, | Inorganic-Copper |
| | | | | Algaecide, | |
| | | | | Molluscicide | |
| Copper sulfate (pentahydrate) | 7758-99-8 | 24401 | 161 | Algaecide, | Inorganic-Copper |
| | | | | Fungicide, | |
| | | | | Insecticide, | |
| | | | | Water | |
| | | | | Treatment, | |
| | | | | Molluscicide, | |
| | | | | Nematicide | |
| Copper sulfate ethylene | NA 1036 | NA 132 | 2480 | N/A | Inorganic-Copper |
| diamine | | | | | |
| Copper sulfate, monohydrate | 10257-54-2 | 24402 | 1789 | Fungicide, | Inorganic-Copper |
| | | | | Algaecide, | |
| | | | | Molluscicide | |
| Copper sulfate, tri-basic | NA 1384 | NA 195 | NA 194 | Herbicide, | Inorganic-Copper |
| | | | | Algaecide, | |
| | | | | Fungicide, | |
| | | | | Water | |
| | | | | Treatment | |
| Copper triethanolamine | 68027-59-6 | NA 125 | 1615 | Algaecide | Inorganic-Copper |
| complex | | | | | |
| Copper-zinc chromate complex | 1344-74-7 | 21003 | 163 | Fungicide, | Inorganic- |
| | | | | Wood | Chromium(VI), |
| | | | | Preservative | Inorganic-Zinc, |
| | | | | | Heavy metal, |
| | | | | | Inorganic-Copper |
| Copper-zinc sulfate complex | 55072-57-6 | 8102 | 164 | Fungicide | Inorganic-Zinc, |
| | | | | | Inorganic-Copper |
| Copper-zinc sulfate complex, | NA 959 | NA 382 | 1751 | Fungicide | Inorganic-Zinc, |
| monohydrate | | | | | Inorganic-Copper |
| Crag turf fungicide 531 | 12001-20-6 | 21006 | NA 171 | Fungicide | Inorganic-Cadmium, |
| (Cd—Cu—Ca—Zn-Chromate) | | | | | Inorganic- |
| | | | | | Chromium(VI), |
| | | | | | Inorganic-Zinc, |
| | | | | | Heavy metal |
| Cryolite | 15096-52-3 | 75101 | 173 | Insecticide | Inorganic |
| Cuprammonium | NA 1382 | NA 195 | NA 194 | Fungicide | Inorganic-Copper |
| Cupric acetoarsenite | 12002-03-8 | 22601 | 2485 | Wood | Inorganic-arsenic, |
| | | | | Preservative | Heavy metal, |
| | | | | | Inorganic-Copper |
| Cupric ferric subsulfate | 12168-20-6 | 42402 | NA 365 | Molluscicide | Inorganic-Copper |
| complex | | | | | |
| Cupric fluosilicate | 12062-24-7 | 75309 | NA 692 | Insecticide | Inorganic-Copper |
| Cupric gluconate | 527-09-3 | 24405 | 3117 | Fungicide | Inorganic-Copper |
| Cupric nitrate | 3251-23-8 | 76102 | 3118 | N/A | Inorganic-Copper |
| Cuprous and cupric oxide, | 82010-82-0 | 42403 | NA 366 | N/A | Inorganic-Copper |
| mixed | | | | | |
| Cuprous chloride (Cu2Cl2) | 7758-89-6 | ##### | 5597 | Fungicide | Inorganic-Copper |
| Cuprous iodide | 7681-65-4 | ##### | NA 902 | N/A | Inorganic-Copper |
| Cuprous sulfide | 22205-45-4 | ##### | NA 124 | N/A | Inorganic-Copper |
| Cuprous thiocyanate | 1111-67-7 | 25602 | 2108 | Microbiocide | Inorganic-Copper |
| Device (swimming pool | NA 1504 | NA 222 | NA 222 | Algaecide | Inorganic-Copper |
| algaecide, copper generating) | | | | | |
| no guarantee required | | | | | |
| Device (swimming pool | NA 1505 | NA 222 | NA 222 | Algaecide | Inorganic |
| algaecide, peroxide) no | | | | | |
| guarantee required | | | | | |
| Device (swimming pool | NA 1506 | NA 222 | NA 222 | Algaecide | Inorganic |
| algaecide, salt in pool) no | | | | | |
| guarantee required | | | | | |
| Di-potassium salts of | 13492-26-7 | 76416 | NA 704 | Fungicide, | Inorganic |
| phosphorous acid | | | | Microbiocide | |
| Diammonium phosphate | 7783-28-0 | NA 819 | 654 | Insecticide, | Inorganic |
| | | | | Fungicide, | |
| | | | | Herbicide, | |
| | | | | Microbiocide, | |
| | | | | pH Adjustment | |
| Diatomaceous earth | 7631-86-9 | 72605 | 195 | Insecticide, | Inorganic |
| | | | | Molluscicide | |
| Diatomaceous earth, other | NA 1155 | NA 173 | 90195 | Insecticide, | Inorganic |
| related | | | | Molluscicide | |
| Dihydrogen | 54453-03-1 | 24406 | NA 200 | N/A | Inorganic-Copper |
| {ethylenediaminetetraacetato(4- | | | | | |
| )}cuprate(2-) | | | | | |
| Disodium EDTA-copper | 14025-15-1 | NA 296 | 3173 | N/A | Inorganic-Copper |
| Disodium mono ethanolamine | 53404-45-8 | 76410 | 1608 | pH Adjustment, | Inorganic |
| phosphate | | | | Fungicide, | |
| | | | | Herbicide, | |
| | | | | Microbiocide | |
| Disodium octaborate | 12008-41-2 | 11107 | 5053 | Insecticide | Inorganic |
| anhydrous | | | | | |
| Disodium octaborate | 12280-03-4 | 11103 | 1800 | Insecticide, | Inorganic |
| tetrahydrate | | | | Herbicide | |
| Disodium phosphate | 7558-79-4 | 76403 | 2534 | pH Adjustment, | Inorganic |
| | | | | Fungicide, | |
| | | | | Herbicide, | |
| | | | | Microbiocide | |
| EDTA diammonium copper | 7379-26-2 | 39117 | 1613 | Algaecide | Inorganic-Copper |
| salt | | | | | |
| EDTA, copper complex | 12276-01-6 | 39105 | 1135 | Algaecide | Inorganic-Copper |
| EDTA, disodium zinc salt | 14025-21-9 | NA 274 | 3189 | N/A | Inorganic-Zinc |
| Fatty acid* - silver complex | 24927-67-1 | 72505 | NA 675 | Microbiocide, | Inorganic-Silver, |
| *(100% C8) | | | | Fungicide, | Heavy metal |
| | | | | Herbicide | |
| Feldspar | 68476-25-5 | NA 118 | 3656 | N/A | Inorganic |
| Ferric chloride | 7705-08-0 | 34901 | 3205 | Herbicide | Inorganic |
| Ferric oxide | 1332-37-2 | NA 104 | 3206 | N/A | Inorganic |
| Ferric sulfate (anhydrous) | 10028-22-5 | 34902 | 1811 | Herbicide | Inorganic |
| Ferrous ammonium sulfate | 10045-89-3 | 50506 | 1307 | Herbicide | Inorganic |
| Ferrous ammonium sulfate | 7783-85-9 | 50509 | NA 421 | Herbicide | Inorganic |
| hexahydrate | | | | | |
| Ferrous sulfate | 7720-78-7 | NA 120 | 289 | Herbicide, | Inorganic |
| | | | | Molluscicide | |
| Ferrous sulfate (monohydrate) | 17375-41-6 | 50507 | 1812 | Herbicide, | Inorganic |
| | | | | Defoliant | |
| Ferrous sulfate heptahydrate | 7782-63-0 | 50502 | 1687 | Herbicide | Inorganic |
| Granite | NA 105 | NA 209 | 3663 | N/A | Inorganic |
| Graphite | 7782-42-5 | NA 210 | 3217 | N/A | Inorganic |
| Hydrated lime | 1302-62-0 | NA 194 | 3669 | pH Adjustment, | Inorganic |
| | | | | Fungicide, | |
| | | | | Microbiocide, | |
| | | | | Herbicide | |
| Hydriodic acid | 10034-85-2 | 46912 | NA 408 | N/A | Inorganic |
| Hydrocyanic acid | 74-90-8 | 45801 | NA 388 | Fumigant | Inorganic |
| Hydrofluoric acid | 7664-39-3 | 45601 | NA 387 | N/A | Inorganic |
| Hydrogen chloride | 7647-01-0 | 45901 | 329 | Fumigant | Inorganic |
| | | | | Fungicide, | |
| | | | | Nematicide, pH | |
| | | | | Adjustment, | |
| | | | | Herbicide | |
| Hydrogen peroxide | 7722-84-1 | 595 | 1794 | Microbiocide, | Inorganic |
| | | | | Fungicide, | |
| | | | | Herbicide, | |
| | | | | Rodenticide | |
| Hydrogen sulfide | 2147416 | NA 227 | NA 227 | Breakdown | Inorganic |
| | | | | product | |
| Hydrophosphorous acid | NA 95 | NA 189 | 2600 | N/A | Inorganic |
| Hydroxymercury cresol | 12379-66-7 | 46001 | NA 390 | Microbiocide | Inorganic-Mercury, |
| | | | | | Heavy metal, Phenols |
| Hypochlorous acid | 7790-92-3 | ##### | NA 110 | Water | Inorganic |
| | | | | Treatment, | |
| | | | | Microbiocide, | |
| | | | | pH Adjustment | |
| Hypoiodous acid | 14332-21-9 | ##### | NA 153 | N/A | Inorganic |
| Inorganic phosphate | NA 940 | NA 169 | 3671 | pH Adjustment, | Inorganic |
| | | | | Fungicide, | |
| | | | | Herbicide, | |
| | | | | Microbiocide | |
| Iodic acid | 7782-68-5 | 46902 | NA 405 | N/A | Inorganic |
| Iodine | 7553-56-2 | 46905 | 718 | Microbiocide, | Inorganic |
| | | | | Fungicide, | |
| | | | | Herbicide | |
| Iron | 7439-89-6 | NA 121 | 655 | N/A | Inorganic |
| Iron oxide (Fe2O3) | 1309-37-1 | ##### | NA 870 | Herbicide | Inorganic |
| Iron oxide pigment | NA 942 | NA 175 | 2618 | Dye | Inorganic |
| Iron phosphate | 10045-86-0 | 34903 | 5014 | Molluscicide | Inorganic |
| Kaolin | 1332-58-7 | ##### | 2629 | Insecticide, | Inorganic |
| | | | | Adjuvant, | |
| | | | | Fungicide, | |
| | | | | Microbiocide | |
| Lauric acid, barium cadmium | 15337-60-7 | NA 118 | 3676 | Adjuvant | Inorganic-Cadmium, |
| salt | | | | | Heavy metal |
| Lead | 7439-92-1 | NA 134 | 2638 | N/A | Inorganic-Lead, |
| | | | | | Heavy metal |
| Lead acetate | 301-04-2 | 48001 | NA 415 | N/A | Inorganic-Lead, |
| | | | | | Heavy metal |
| Lead arsenate | 7784-40-9 | 13503 | 353 | Herbicide, | Inorganic-arsenic, |
| | | | | Insecticide, | Inorganic-lead, |
| | | | | Rodenticide | Heavy metal |
| Lead arsenate, basic | 1327-31-7 | 13502 | 354 | Herbicide, | Inorganic-arsenic, |
| | | | | Insecticide, | Inorganic-lead, |
| | | | | Rodenticide | Heavy metal |
| Lead metasilicate | NA 938 | NA 138 | 1106 | N/A | Inorganic-Lead, |
| | | | | | Heavy metal |
| Lead monoxide | 10190-55-3 | NA 123 | 1271 | N/A | Inorganic-Lead, |
| | | | | | Heavy metal |
| Lead naphthenate | 61790-14-5 | 48004 | 3296 | N/A | Inorganic-Lead, |
| | | | | | Heavy metal |
| Lignin sulfonic acid, copper | NA 933 | NA 126 | 1925 | N/A | Inorganic-Copper |
| salt | | | | | |
| Lignin sulfonic acid, zinc salt | NA 1014 | NA 102 | 1768 | N/A | Inorganic-Zinc |
| Lignin sulfonic acid, zinc, | NA 1030 | NA 126 | 1771 | N/A | Inorganic-Zinc |
| manganese & iron salts | | | | | |
| Lime | 1305-78-8 | 75604 | 977 | pH Adjustment, | Inorganic |
| | | | | Fungicide, | |
| | | | | Herbicide, | |
| | | | | Microbiocide | |
| Lime phosphate | NA 1432 | NA 200 | NA 199 | Plant Growth | Inorganic |
| | | | | Regulator | |
| Lime-sulfur | 1344-81-6 | 76702 | 358 | Insecticide, | Inorganic |
| | | | | Fungicide | |
| Litharge | 1317-36-8 | 48002 | NA 416 | N/A | Inorganic-Lead, |
| | | | | | Heavy metal |
| Lithium carbonate | 554-13-2 | NA 119 | 3924 | pH Adjustment, | Inorganic |
| | | | | Fungicide, | |
| | | | | Microbiocide, | |
| | | | | Herbicide | |
| Lithium chloride | 7447-41-8 | ##### | 5318 | N/A | Inorganic |
| Lithium hydroxide | 1310-65-2 | NA 143 | 3267 | pH Adjustment, | Inorganic |
| | | | | Fungicide, | |
| | | | | Herbicide, | |
| | | | | Microbiocide | |
| Lithium hypochlorite | 13840-33-0 | 14702 | 922 | Microbiocide, | Inorganic |
| | | | | Water | |
| | | | | Treatment | |
| Lithium perchlorate | 2150251 | NA 115 | 3268 | N/A | Inorganic |
| Magnesium aluminum | 1327-43-1 | 75310 | NA 693 | Insecticide | Inorganic |
| fluosilicate | | | | | |
| Magnesium arsenate | 10103-50-1 | 13504 | NA 126 | Herbicide, | Inorganic-arsenic, |
| | | | | Insecticide, | Heavy metal |
| | | | | Rodenticide | |
| Magnesium carbonate | 546-93-0 | 73503 | 2654 | pH Adjustment, | Inorganic |
| | | | | Fungicide, | |
| | | | | Microbiocide, | |
| | | | | Herbicide | |
| Magnesium chlorate | 10326-21-3 | ##### | NA 155 | Defoliant, | Inorganic |
| | | | | Herbicide, | |
| | | | | Microbiocide | |
| Magnesium chloride | 7786-30-3 | 13902 | 365 | Molluscicide, | Inorganic |
| | | | | Herbicide, | |
| | | | | Insecticide, | |
| | | | | Microbiocide | |
| Magnesium chloride, | 7791-18-6 | NA 110 | 2655 | Molluscicide, | Inorganic |
| hexahydrate | | | | Herbicide, | |
| | | | | Insecticide, | |
| | | | | Microbiocide | |
| Magnesium fluosilicate | 16949-65-8 | 75304 | NA 689 | Insecticide | Inorganic |
| Magnesium lime | NA 931 | NA 111 | 3679 | N/A | Inorganic |
| Magnesium nitrate | 10377-60-3 | NA 115 | 3269 | Rodenticide, | Inorganic |
| | | | | Microbiocide | |
| Magnesium oxide | 1309-48-4 | 9235 | 2656 | pH Adjustment, | Inorganic |
| | | | | Fungicide, | |
| | | | | Herbicide, | |
| | | | | Microbiocide | |
| Magnesium phosphide | 12057-74-8 | 66504 | 2085 | Fumigant, | Inorganic |
| | | | | Rodenticide | |
| Magnesium silicate | 1343-88-0 | 72601 | NA 680 | Insecticide, | Inorganic |
| | | | | Adjuvant, | |
| | | | | Fungicide, | |
| | | | | Microbiocide | |
| Magnesium silicate, hydrous | 14807-96-6 | NA 113 | 2658 | Adjuvant | Inorganic |
| Magnesium sulfate | 7487-88-9 | 50503 | 1339 | Molluscicide, | Inorganic |
| | | | | Adjuvant | |
| Malachite (copper equivalent | 1319-53-5 | ##### | NA 155 | N/A | Inorganic-Copper |
| 57%) | | | | | |
| Mancozeb | 2233100 | 14504 | 211 | Fungicide | Dithiocarbamate, |
| | | | | | Inorganic-Zinc |
| Maneb and nickel sulfate | 8005-46-7 | ##### | NA 158 | Fungicide | Dithiocarbamate, |
| hexahydrate (014505 + 050505) | | | | | Inorganic-Nickel |
| Manganese (II) oxide | 1344-43-0 | NA 104 | 3274 | N/A | Inorganic |
| Manganese arsenate | 7784-38-5 | 13506 | NA 127 | Herbicide, | Inorganic-arsenic, |
| | | | | Insecticide, | Heavy metal |
| | | | | Rodenticide | |
| Manganese carbamate | NA 55 | NA 103 | 3273 | N/A | Inorganic |
| Manganese sulfate | 7785-87-7 | NA 105 | 658 | N/A | Inorganic |
| Manganous oxide | 1344-43-0 | NA 143 | 3275 | N/A | Inorganic |
| Mercuric acetate | 1600-27-7 | 52104 | NA 441 | N/A | Inorganic-Mercury, |
| | | | | | Heavy metal |
| Mercuric chloride | 7487-94-7 | 52001 | 372 | Fungicide | Inorganic-Mercury, |
| | | | | | Heavy metal |
| Mercuric dimethyl | 15415-64-2 | 34808 | 709 | Microbiocide | Inorganic-Mercury, |
| dithiocarbamate | | | | | Heavy metal, |
| | | | | | Dithiocarbamate |
| Mercuric iodide | 7774-29-0 | 52003 | NA 438 | N/A | Inorganic-Mercury, |
| | | | | | Heavy metal |
| Mercuric oleate | 1191-80-6 | 52105 | 3276 | N/A | Inorganic-Mercury, |
| | | | | | Heavy metal |
| Mercuric oxide | 21908-53-2 | 52102 | 955 | Fungicide | Inorganic-Mercury, |
| | | | | | Heavy metal |
| Mercury | 7439-97-6 | 52301 | NA 26 | N/A | Inorganic-Mercury, |
| | | | | | Heavy metal |
| Mercury naphthenate | 1336-96-5 | 52101 | NA 439 | Wood | Inorganic-Mercury, |
| | | | | Preservative, | Heavy metal |
| | | | | Insecticide, | |
| | | | | Fungicide | |
| Mercury pentanedione | 14024-55-6 | 52103 | NA 440 | N/A | Inorganic-Mercury, |
| | | | | | Heavy metal |
| Mercury phenate | 588-66-9 | 52106 | NA 442 | N/A | Inorganic-Mercury, |
| | | | | | Heavy metal |
| Metallic silver | 7440-22-4 | 72501 | 2125 | Microbiocide, | Inorganic-Silver, |
| | | | | Fungicide, | Heavy metal |
| | | | | Herbicide | |
| Metiram | 9006-42-2 | 14601 | 493 | Fungicide | Dithiocarbamate, |
| | | | | | Inorganic-Zinc |
| Mica | 12003-33-2 | NA 118 | 3690 | N/A | Inorganic |
| Mixed copper chelates or | NA 1437 | NA 213 | NA 212 | Fungicide | Inorganic-Copper |
| complex blend of copper salts | | | | | |
| Molybdenum | 7439-98-7 | NA 152 | 3903 | N/A | Inorganic |
| Molybdic acid, disodium salt | 7631-95-0 | NA 135 | 2690 | N/A | Inorganic |
| Mono-potassium salts of | 13977-65-6 | 76416 | NA 705 | Fungicide, | Inorganic |
| phosphorous acid | | | | Microbiocide | |
| Monosodium phosphate | 7558-80-7 | 76409 | 1772 | pH Adjustment, | Inorganic |
| | | | | Fungicide, | |
| | | | | Herbicide, | |
| | | | | Microbiocide | |
| N-(2-hydroxyethyl) ethylene | NA 1025 | NA 122 | 1130 | N/A | Inorganic-Copper |
| diamine triacetic acid, copper | | | | | |
| salt | | | | | |
| Neodecanoic acid, copper salt | 50315-14-5 | 97503 | NA 854 | N/A | Inorganic-Copper |
| Nickel | 7440-02-0 | NA 121 | 762 | N/A | Inorganic-Nickel |
| Nickel diethyl hexyl acid | NA 928 | NA 53 | 3696 | N/A | Inorganic-Nickel |
| phosphate complex | | | | | |
| Nickel sulfate (anhydrous) | 7786-81-4 | 50508 | NA 420 | Fungicide | Inorganic-Nickel |
| Nickel sulfate hexahydrate | 10101-97-0 | 50505 | NA 419 | Fungicide | Inorganic-Nickel |
| Nitric acid | 7697-37-2 | 45951 | NA 389 | N/A | Inorganic |
| Nitric acid technical | 7697-37-2 | NA 47 | 2703 | N/A | Inorganic |
| Nitrogen trichloride | 10025-85-1 | NA 50 | 2705 | N/A | Inorganic |
| Nitrogen, liquified | 7727-37-9 | ##### | 2267 | Insecticide | Inorganic |
| Nitrous oxide | 10024-97-2 | NA 115 | 3302 | N/A | Inorganic |
| Noury dry cobalt 12% | NA 21 | NA 33 | 2712 | N/A | Inorganic |
| Nuxtra manganese 12% | NA 746 | NA 135 | 2719 | N/A | Inorganic |
| catalyst | | | | | |
| o-Phenylphenol, alkyl* amine- | 82010-74-0 | 64107 | NA 533 | Microbiocide | Phenols, Inorganic- |
| zinc salt of *(100% C18) | | | | | Zinc |
| Oxalic acid | 144-62-7 | 9601 | 899 | Microbiocide | Inorganic |
| Ozone | 10028-15-6 | NA 228 | NA 228 | Water | Inorganic |
| | | | | Treatment, | |
| | | | | Microbiocide | |
| p-tert- | 53433-01-5 | 52002 | NA 437 | Microbiocide | Inorganic-Mercury, |
| Octylphenoxyethoxyethyl | | | | | Heavy metal |
| dimethyl benzyl ammonium | | | | | |
| mercuric chloride | | | | | |
| Paris green | 12002-03-8 | 22601 | 460 | Wood | Inorganic-Arsenic, |
| | | | | Preservative | Inorganic-Copper, |
| | | | | | Heavy metal |
| Penta potassium triphosphate | 13845-36-8 | 76412 | 3319 | N/A | Inorganic |
| Pentachlorophenol, zinc salt | 2917-32-0 | 63008 | NA 510 | Wood | Chlorinated Phenol, |
| | | | | Preservative, | Inorganic-Zinc |
| | | | | Microbiocide, | |
| | | | | Algaecide, | |
| | | | | Fungicide | |
| Perboric acid, sodium salt | NA 1160 | NA 174 | 5452 | Insecticide | Inorganic |
| Perboric acid, sodium salt, | 10332-33-9 | 11105 | 5052 | Insecticide | Inorganic |
| monohydrate | | | | | |
| Perchloric acid, calcium salt | 13477-36-6 | NA 670 | 3321 | N/A | Inorganic |
| Perlite | NA 756 | NA 136 | 2767 | N/A | Inorganic |
| Peroxyacetic acid | 79-21-0 | 63201 | 2291 | Microbiocide, | Inorganic |
| | | | | Fungicide, | |
| | | | | Herbicide, | |
| | | | | Rodenticide | |
| Peroxydisulfuric acid | 13445-49-3 | 63601 | NA 524 | N/A | Inorganic |
| Phosphine | 7803-51-2 | 66500 | 3541 | Fumigant, | Inorganic |
| | | | | Insecticide | |
| Phosphoric acid | 7664-38-2 | 76001 | 871 | Fungicide, pH | Inorganic |
| | | | | Adjustment, | |
| | | | | Herbicide, | |
| | | | | Microbiocide | |
| Phosphoric acid, disodium salt, | 10028-24-7 | 76415 | NA 703 | pH Adjustment, | Inorganic |
| dihydrate | | | | Fungicide, | |
| | | | | Herbicide, | |
| | | | | Microbiocide | |
| Phosphoric acid, | 7778-77-0 | 76413 | 2777 | pH Adjustment, | Inorganic |
| monopotassium salt | | | | Fungicide, | |
| | | | | Herbicide, | |
| | | | | Microbiocide | |
| Phosphoric acid, trisodium salt | 56802-99-4 | ##### | 3851 | Water | Inorganic |
| (chlorinated) | | | | Treatment | |
| Phosphorous acid | 13598-36-2 | 76002 | NA 699 | Fungicide, | Inorganic |
| | | | | Microbiocide | |
| Phosphorus | 7723-14-0 | 66502 | 513 | N/A | Inorganic |
| Phosphorus pentasulfide | 1314-56-3 | NA 640 | 2778 | N/A | Inorganic |
| Polyoxin D zinc salt | 146659-78-1 | ##### | NA 129 | Fungicide | Inorganic-Zinc |
| Potassium aluminum silicate | NA 996 | NA 774 | 3344 | Adjuvant | Inorganic |
| Potassium aluminum sulfate | 7784-24-9 | NA 775 | 3345 | Insecticide, | Inorganic |
| | | | | Herbicide | |
| Potassium arsenite | 10124-50-2 | 13605 | NA 131 | Herbicide, | Inorganic-arsenic, |
| | | | | Insecticide, | Heavy metal |
| | | | | Rodenticide | |
| Potassium arsenite | 13464-35-2 | 13605 | NA 132 | Herbicide, | Inorganic-arsenic, |
| | | | | Insecticide, | Heavy metal |
| | | | | Rodenticide | |
| Potassium azide | 20762-60-1 | ##### | NA 898 | N/A | Inorganic |
| Potassium bicarbonate | 298-14-6 | 73508 | 5037 | Fungicide | Inorganic |
| Potassium bifluoride | 7789-29-9 | 75203 | NA 687 | N/A | Inorganic |
| Potassium bisulfate | 7646-93-7 | 5605 | 1860 | pH Adjustment, | Inorganic |
| | | | | Fungicide, | |
| | | | | Herbicide, | |
| | | | | Microbiocide | |
| Potassium bromide | 2138164 | 13903 | 1090 | Microbiocide | Inorganic |
| Potassium carbonate | 584-08-7 | 73504 | 1620 | pH Adjustment, | Inorganic |
| | | | | Fungicide, | |
| | | | | Microbiocide, | |
| | | | | Herbicide | |
| Potassium chlorate | 696616 | 73303 | 3347 | Defoliant, | Inorganic |
| | | | | Herbicide, | |
| | | | | Microbiocide | |
| Potassium chloride | 7447-40-7 | 13904 | NA 137 | N/A | Inorganic |
| Potassium chromate | 7789-00-6 | 68301 | 700 | Wood | Inorganic- |
| | | | | Preservative | Chromium(VI), |
| | | | | | Heavy metal |
| Potassium cyanate | 590-28-3 | 68002 | NA 600 | N/A | Inorganic |
| Potassium cyanide | 151-50-8 | ##### | 497 | Rodenticide | Inorganic |
| Potassium dichromate | 7778-50-9 | 68302 | 2167 | Wood | Inorganic- |
| | | | | Preservative | Chromium(VI) |
| Potassium hexafluoroarsenate | 17029-22-0 | ##### | NA 160 | N/A | Inorganic-arsenic, |
| | | | | | Heavy metal |
| Potassium hydroxide | 1310-58-3 | 75602 | 1306 | pH Adjustment, | Inorganic |
| | | | | Fungicide, | |
| | | | | Herbicide, | |
| | | | | Microbiocide | |
| Potassium hypochlorite | 7778-66-7 | ##### | NA 110 | Microbiocide | Inorganic |
| Potassium iodate | 2138256 | 75703 | NA 696 | N/A | Inorganic |
| Potassium iodate (KH(IO3)2) | 13455-24-8 | 75704 | NA 697 | N/A | Inorganic |
| Potassium iodide | 7681-11-0 | 75701 | 1003 | Microbiocide | Inorganic |
| Potassium mercuric iodide | 7783-33-7 | 52107 | NA 443 | N/A | Inorganic-Mercury, |
| | | | | | Heavy metal |
| Potassium monopersulfate | 10361-76-9 | ##### | NA 104 | N/A | Inorganic |
| Potassium nitrate | 7757-79-1 | 76103 | 726 | Rodenticide, | Inorganic |
| | | | | Microbiocide | |
| Potassium nitrite | 7758-09-0 | 76203 | 3356 | Rodenticide, | Inorganic |
| | | | | Microbiocide | |
| Potassium permanganate | 7722-64-7 | 68501 | 498 | Fungicide, | Inorganic |
| | | | | Algaecide, | |
| | | | | Molluscicide, | |
| | | | | Microbiocide | |
| Potassium peroxydisulfate | 7727-21-1 | 63602 | 1028 | Microbiocide, | Inorganic |
| | | | | Fungicide, | |
| | | | | Herbicide, | |
| | | | | Rodenticide | |
| Potassium peroxymonosulfate | 10058-23-8 | 63604 | 1859 | Microbiocide, | Inorganic |
| | | | | Fungicide, | |
| | | | | Herbicide, | |
| | | | | Rodenticide | |
| Potassium peroxymonosulfate | 37222-66-5 | 63607 | NA 527 | Microbiocide, | Inorganic |
| sulfate, | | | | Fungicide, | |
| (2KHSO5.KHSO4.K2SO4) | | | | Herbicide, | |
| | | | | Rodenticide | |
| Potassium peroxymonosulfate | 70693-62-8 | 63607 | NA 528 | Microbiocide, | Inorganic |
| sulfate, | | | | Fungicide, | |
| (2KHSO5.KHSO4.K2SO4) | | | | Herbicide, | |
| | | | | Rodenticide | |
| Potassium phosphate | 7778-53-2 | 76407 | 3358 | pH Adjustment, | Inorganic |
| | | | | Fungicide, | |
| | | | | Herbicide, | |
| | | | | Microbiocide | |
| Potassium phosphate, dibasic | 2138438 | ##### | 3359 | pH Adjustment, | Inorganic |
| | | | | Fungicide, | |
| | | | | Herbicide, | |
| | | | | Microbiocide | |
| Potassium phosphite | NA 1565 | NA 229 | 5766 | Fungicide | Inorganic |
| Potassium phosphonate | NA 1563 | NA 227 | NA 227 | Fungicide | Inorganic |
| (phosphorous acid equivalent) | | | | | |
| Potassium polysulfide | 37199-66-9 | 76703 | NA 708 | Insecticide, | Inorganic |
| | | | | Fungicide | |
| Potassium pyrophosphate | 7320-34-5 | 76408 | 1175 | pH Adjustment, | Inorganic |
| | | | | Fungicide, | |
| | | | | Herbicide, | |
| | | | | Microbiocide | |
| Potassium silicate | 1312-76-1 | 72606 | 2826 | Insecticide, | Inorganic |
| | | | | Adjuvant, | |
| | | | | Fungicide, | |
| | | | | Microbiocide | |
| Potassium sulfate | 7778-80-5 | 5603 | 3362 | Insecticide, | Inorganic |
| | | | | Herbicide | |
| Potassium sulfite | 10117-38-1 | NA 785 | 3364 | Fungicide | Inorganic |
| Potassium tetraborate | 1332-77-0 | NA 144 | 3365 | Insecticide | Inorganic |
| Potassium tetrathionate | 13932-13-3 | 75903 | NA 698 | Insecticide, | Inorganic |
| | | | | Herbicide | |
| Potassium thiocyanate | 333-20-0 | 68201 | NA 605 | Insecticide | Inorganic |
| Potassium thiosulfate | 10233-00-8 | 80102 | NA 773 | Insecticide, | Inorganic |
| | | | | Fungicide, | |
| | | | | Herbicide | |
| Potassium triiodide | 12298-68-9 | 46917 | 5066 | Microbiocide | Inorganic |
| Propineb | 12071-83-9 | ##### | NA 155 | Fungicide, | Dithiocarbamate, |
| | | | | Microbiocide | Inorganic-Zinc |
| Pumice | 1332-09-8 | NA 150 | 3777 | N/A | Inorganic |
| Quartz sand | NA 1363 | NA 193 | NA 192 | Insect | Inorganic |
| | | | | Repellent | |
| Rock powder | NA 1375 | NA 194 | NA 194 | N/A | Inorganic |
| Selenium and compounds | 7782-49-2 | 72001 | NA 31 | N/A | Inorganic |
| Selenium disulfide | 7488-56-4 | 72003 | NA 673 | N/A | Inorganic |
| Shale | NA 459 | NA 821 | 3794 | N/A | Inorganic |
| Silica aerogel | 63231-67-4 | 72602 | 529 | Insecticide, | Inorganic |
| | | | | Adjuvant, | |
| | | | | Fungicide, | |
| | | | | Microbiocide | |
| Silica gel | 63231-67-4 | 72602 | 3557 | Insecticide, | Inorganic |
| | | | | Adjuvant, | |
| | | | | Fungicide, | |
| | | | | Microbiocide | |
| Silica hydrate | NA 461 | NA 824 | 2853 | N/A | Inorganic |
| Silica, crystalline-fused | 60676-86-0 | NA 823 | 2851 | N/A | Inorganic |
| Silica, crystalline-quartz | 14808-60-7 | NA 138 | 2852 | N/A | Inorganic |
| Silicic acid | 1343-98-2 | NA 825 | 3556 | N/A | Inorganic |
| Silicon dioxide fresh water | NA 1524 | NA 223 | NA 223 | Insecticide | Inorganic |
| fossils | | | | | |
| Silver acetate | 563-63-3 | 72507 | NA 676 | Microbiocide, | Inorganic-Silver, |
| | | | | Fungicide, | Heavy metal |
| | | | | Herbicide | |
| Silver carbonate | 534-16-7 | 72509 | NA 678 | Microbiocide, | Inorganic-Silver, |
| | | | | Fungicide, | Heavy metal |
| | | | | Herbicide | |
| Silver chloride | 7783-90-6 | 72506 | 2324 | Microbiocide, | Inorganic-Silver, |
| | | | | Fungicide, | Heavy metal |
| | | | | Herbicide | |
| Silver copper zeolite | 130328-19-7 | ##### | NA 110 | Microbiocide | Inorganic-Silver, |
| | | | | | Heavy metal, |
| | | | | | Inorganic-Copper |
| Silver fluoride | 7775-41-9 | 72502 | NA 674 | Microbiocide, | Inorganic-Silver, |
| | | | | Fungicide, | Heavy metal |
| | | | | Herbicide | |
| Silver iodide, colloidal | 7783-96-2 | NA 124 | 1556 | Microbiocide, | Inorganic-Silver, |
| | | | | Fungicide, | Heavy metal |
| | | | | Herbicide | |
| Silver nitrate | 7761-88-8 | 72503 | 2856 | Microbiocide, | Inorganic-Silver, |
| | | | | Fungicide, | Heavy metal |
| | | | | Herbicide, | |
| | | | | Plant Growth | |
| | | | | Regulator | |
| Silver orthophosphate | 7784-09-0 | 72510 | NA 679 | Microbiocide, | Inorganic-Silver, |
| (Ag3PO4) | | | | Fungicide, | Heavy metal |
| | | | | Herbicide | |
| Silver oxide (Ag4O4) | 1301-96-8 | ##### | NA 111 | Microbiocide, | Inorganic-Silver, |
| | | | | Fungicide, | Heavy metal |
| | | | | Herbicide | |
| Silver oxide (Ag4O4) | 155645-89-9 | ##### | NA 111 | Microbiocide, | Inorganic-Silver, |
| | | | | Fungicide, | Heavy metal |
| | | | | Herbicide | |
| Silver sodium hydrogen | NA 1166 | 72560 | NA 164 | Microbiocide | Inorganic-Silver, |
| zirconium phosphate | | | | | Heavy metal |
| (Ag0.18Na0.57H0.25Zr2(PO4)3) | | | | | |
| Silver thiocyanate | 1701-93-5 | 68203 | NA 606 | Microbiocide, | Inorganic-Silver, |
| | | | | Fungicide, | Heavy metal |
| | | | | Herbicide | |
| Silver thiuronium acrylate copolymer | 53404-00-5 | 72701 | NA 681 | Microbiocide, | Inorganic-Silver, |
| | | | | Fungicide, | Heavy metal |
| | | | | Herbicide | |
| Silver zeolite | 130328-18-6 | ##### | NA 125 | Microbiocide | Inorganic-Silver, |
| | | | | | Heavy metal |
| Silver zinc zeolite | 130328-20-0 | ##### | NA 111 | Microbiocide | Inorganic-Silver, |
| | | | | | Inorganic-Zinc, |
| | | | | | Heavy metal |
| Silver, ionic | 14701-21-4 | NA 169 | 5150 | Microbiocide, | Inorganic-Silver, |
| | | | | Fungicide, | Heavy metal |
| | | | | Herbicide | |
| Soapstone | NA 465 | NA 829 | 3797 | N/A | Inorganic |
| Sodium | 7440-23-5 | NA 830 | 2858 | N/A | Inorganic |
| Sodium acid phosphate | 7558-80-7 | 76409 | 1413 | pH Adjustment, | Inorganic |
| | | | | Fungicide, | |
| | | | | Herbicide, | |
| | | | | Microbiocide | |
| Sodium acid pyrophosphate | 7758-16-9 | NA 831 | 3381 | pH Adjustment | Inorganic |
| Sodium aluminate | 1302-42-7 | NA 145 | 3382 | N/A | Inorganic |
| Sodium aluminum fluosilicate | 53404-77-6 | 75308 | NA 691 | Insecticide | Inorganic |
| Sodium aluminum phosphate | 7785-88-8 | NA 832 | 3383 | N/A | Inorganic |
| Sodium arsenate | 13464-38-5 | 13505 | 283 | Herbicide, | Inorganic-arsenic, |
| | | | | Insecticide, | Heavy metal |
| | | | | Rodenticide | |
| Sodium arsenite | 7784-46-5 | 13603 | 534 | Herbicide, | Inorganic-arsenic, |
| | | | | Insecticide, | Heavy metal |
| | | | | Rodenticide, | |
| | | | | Fungicide | |
| Sodium azide | 26628-22-8 | ##### | NA 897 | N/A | Inorganic |
| Sodium bicarbonate | 144-55-8 | 73505 | 1134 | Fungicide, pH | Inorganic |
| | | | | Adjustment | |
| Sodium bichromate dihydrate | 7789-12-0 | 68306 | 3980 | Wood | Inorganic- |
| | | | | Preservative | Chromium(VI), |
| | | | | | Inorganic |
| Sodium bifluoride | 1333-83-1 | 75201 | 3385 | N/A | Inorganic |
| Sodium binoxalate | 1186-49-8 | 9602 | NA 110 | Microbiocide | Inorganic |
| Sodium bisulfate | 7681-38-1 | 73201 | 905 | pH Adjustment, | Inorganic |
| | | | | Fungicide, | |
| | | | | Herbicide, | |
| | | | | Microbiocide | |
| Sodium bisulfite | 7631-90-5 | 78201 | 547 | pH Adjustment, | Inorganic |
| | | | | Fungicide | |
| Sodium borohydride | 16940-66-2 | NA 834 | 3387 | N/A | Inorganic |
| Sodium bromate | 7789-38-0 | 13908 | NA 138 | N/A | Inorganic |
| Sodium bromide | 7647-15-6 | 13907 | 1103 | Molluscicide, | Inorganic |
| | | | | Herbicide, | |
| | | | | Insecticide, | |
| | | | | Microbiocide | |
| Sodium carbonate | 497-19-8 | 73506 | 854 | pH Adjustment, | Inorganic |
| | | | | Fungicide, | |
| | | | | Microbiocide, | |
| | | | | Herbicide | |
| Sodium chlorate | 2144591 | 73301 | 536 | Defoliant, | Inorganic |
| | | | | Herbicide, | |
| | | | | Microbiocide | |
| Sodium chloride | 7647-14-5 | 13905 | 721 | Molluscicide, | Inorganic |
| | | | | Herbicide, | |
| | | | | Insecticide, | |
| | | | | Microbiocide | |
| Sodium chlorite | 7758-19-2 | 20502 | 2148 | Microbiocide, | Inorganic |
| | | | | Water | |
| | | | | Treatment | |
| Sodium chromate | 2144646 | 68303 | 1241 | Wood | Inorganic- |
| | | | | Preservative | Chromium(VI), |
| | | | | | Heavy metal |
| Sodium cyanide | 143-33-9 | 74002 | 688 | Rodenticide | Inorganic |
| Sodium dichromate | 10588-01-9 | 68304 | 3395 | Wood | Inorganic- |
| | | | | Preservative | Chromium(VI), |
| | | | | | Heavy metal |
| Sodium dihydrogen phosphate | 7558-80-7 | 76409 | 1022 | pH Adjustment, | Inorganic |
| | | | | Fungicide, | |
| | | | | Herbicide, | |
| | | | | Microbiocide | |
| Sodium fluoride | 7681-49-4 | 75202 | 537 | Wood | Inorganic |
| | | | | Preservative, | |
| | | | | Insecticide | |
| Sodium fluosilicate | 16893-85-9 | 75306 | 538 | Insecticide | Inorganic |
| Sodium hexametaphosphate | 10124-56-8 | 76402 | 1623 | pH Adjustment | Inorganic |
| Sodium hydrosulfite | 7775-14-6 | 78202 | 3408 | pH Adjustment | Inorganic |
| Sodium hydroxide | 1310-73-2 | 75603 | 362 | pH Adjustment, | Inorganic |
| | | | | Fungicide, | |
| | | | | Herbicide, | |
| | | | | Microbiocide | |
| Sodium hypobromite | NA 1442 | NA 214 | NA 213 | Microbiocide | Inorganic |
| Sodium hypochlorite | 7681-52-9 | 14703 | 539 | Microbiocide, | Inorganic |
| | | | | Water | |
| | | | | Treatment, | |
| | | | | Nematicide | |
| Sodium hypophosphate | 13721-43-2 | NA 848 | 3409 | N/A | Inorganic |
| Sodium hypophosphite | 7681-53-0 | NA 849 | 3410 | N/A | Inorganic |
| Sodium iodide | 7681-82-5 | 75702 | NA 695 | N/A | Inorganic |
| Sodium metabisulfite | 7681-57-4 | NA 191 | NA 190 | Fungicide | Inorganic |
| Sodium metaborate | 7775-19-1 | 11104 | 689 | Insecticide | Inorganic |
| Sodium metaborate octahydrate | NA 1045 | NA 154 | 4012 | Insecticide | Inorganic |
| Sodium metaborate | NA 1046 | NA 154 | 4013 | Insecticide | Inorganic |
| tetrahydrate | | | | | |
| Sodium metasilicate | 6834-92-0 | 72604 | 940 | Insecticide, | Inorganic |
| | | | | Adjuvant, | |
| | | | | Fungicide, | |
| | | | | Microbiocide | |
| Sodium metasilicate, | 6834-92-0 | 72604 | 1840 | Insecticide, | Inorganic |
| anhydrous | | | | Adjuvant, | |
| | | | | Fungicide, | |
| | | | | Microbiocide | |
| Sodium molybdate | 7631-95-0 | NA 855 | 1761 | N/A | Inorganic |
| Sodium nitrate | 7631-99-4 | 76104 | 696 | Rodenticide, | Inorganic |
| | | | | Microbiocide | |
| Sodium nitrite | 7632-00-0 | 76204 | 2862 | Rodenticide, | Inorganic |
| | | | | Microbiocide | |
| Sodium oxalate | 62-76-0 | 9604 | NA 111 | Microbiocide | Inorganic |
| Sodium pentaborate | 11139-65-4 | NA 862 | 1347 | Insecticide, | Inorganic |
| | | | | Plant Growth | |
| | | | | Regulator | |
| Sodium perborate | 2092204 | ##### | 3429 | Insecticide | Inorganic |
| Sodium perborate (NaBO3) | 10486-00-7 | 11108 | NA 115 | Insecticide | Inorganic |
| tetrahydrate | | | | | |
| Sodium permanganate | 10101-50-5 | NA 145 | 3430 | N/A | Inorganic |
| Sodium peroxide (Na2(O2)) | 1313-60-6 | 63606 | NA 526 | Microbiocide, | Inorganic |
| | | | | Fungicide, | |
| | | | | Herbicide, | |
| | | | | Rodenticide | |
| Sodium persulfate | 7775-27-1 | 63603 | 2336 | N/A | Inorganic |
| Sodium phosphate | 7558-79-4 | 76403 | 1790 | pH Adjustment, | Inorganic |
| | | | | Fungicide, | |
| | | | | Herbicide, | |
| | | | | Microbiocide | |
| Sodium phosphate, monobasic | 7558-80-7 | 76409 | 3433 | pH Adjustment, | Inorganic |
| | | | | Fungicide, | |
| | | | | Herbicide, | |
| | | | | Microbiocide | |
| Sodium polysulfide | 1344-08-7 | 6902 | 541 | Insecticide, | Inorganic |
| | | | | Fungicide | |
| Sodium pyroarsenate | 13464-42-1 | 13401 | 2864 | Wood | Inorganic-arsenic, |
| | | | | Preservative, | Heavy metal |
| | | | | Fungicide | |
| Sodium selenate | 13410-01-0 | 72002 | NA 672 | N/A | Inorganic |
| Sodium sesquicarbonate | 533-96-0 | 73507 | 1292 | pH Adjustment, | Inorganic |
| | | | | Fungicide, | |
| | | | | Microbiocide, | |
| | | | | Herbicide | |
| Sodium silicate | 1344-09-8 | 72603 | 2865 | Insecticide, | Inorganic |
| | | | | Adjuvant, | |
| | | | | Fungicide, | |
| | | | | Microbiocide | |
| Sodium silicoaluminate | NA 1005 | NA 868 | 2866 | Adjuvant | Inorganic |
| Sodium silver thiosulfate | NA 1398 | NA 197 | NA 196 | Herbicide | Inorganic-Silver, |
| | | | | | Heavy metal |
| Sodium sulfate | 7757-82-6 | 5604 | 1532 | Insecticide, | Inorganic |
| | | | | Herbicide | |
| Sodium sulfide | 1313-82-2 | NA 214 | NA 213 | N/A | Inorganic |
| Sodium sulfite | 7757-83-7 | 78203 | 1635 | Fungicide | Inorganic |
| Sodium tetraborate (anhydrous) | 1330-43-4 | 11112 | 5054 | Insecticide, | Inorganic |
| | | | | Herbicide, | |
| | | | | Molluscicide | |
| Sodium tetraborate | 11130-12-4 | NA 871 | 1808 | Insecticide | Inorganic |
| (pentahydrate) | | | | | |
| Sodium tetrathiocarbonate | 7345-69-9 | ##### | 2273 | Fumigant, | Inorganic |
| | | | | Fungicide, | |
| | | | | Nematicide | |
| Sodium thioarsenate | 17367-56-5 | 13507 | NA 128 | N/A | Inorganic-arsenic, |
| (Na3AsO3S) | | | | | Heavy metal |
| Sodium thiocyanate | 540-72-7 | 68202 | 848 | Herbicide | Inorganic |
| Sodium thiosulfate | 7772-98-7 | 80104 | 332 | Insecticide, | Inorganic |
| | | | | Fungicide, | |
| | | | | Herbicide | |
| Sodium tripolyphosphate | 7758-29-4 | 76404 | 1024 | pH Adjustment | Inorganic |
| Sulfur | 7704-34-9 | 77501 | 560 | Fungicide, | Inorganic |
| | | | | Insecticide | |
| Sulfur chloride | 10025-67-9 | NA 116 | 3461 | N/A | Inorganic |
| Sulfur dichloride | 10545-99-0 | NA 915 | 3462 | N/A | Inorganic |
| Sulfur dioxide | 2024422 | 77601 | 561 | Dessicant | Inorganic |
| Sulfuric acid | 7664-93-9 | 78001 | 442 | Herbicide, | Inorganic |
| | | | | Dessicant, | |
| | | | | Fungicide, | |
| | | | | Microbiocide, | |
| | | | | pH Adjustment | |
| Sulfuryl chloride | 7791-25-5 | 78002 | NA 721 | N/A | Inorganic |
| Sulfuryl fluoride | 2699-79-8 | 78003 | 618 | Fumigant | Inorganic |
| Talc | 14807-96-6 | NA 923 | 2911 | N/A | Inorganic |
| Tetracopper calcium | 1336-15-8 | 23502 | NA 187 | Fungicide | Inorganic-Copper |
| oxychloride | | | | | |
| Tetrapotassium pyrophosphate | 7320-34-5 | 76408 | 1918 | pH Adjustment, | Inorganic |
| | | | | Fungicide, | |
| | | | | Herbicide, | |
| | | | | Microbiocide | |
| Tetrasodium pyrophosphate | 7722-88-5 | 76405 | 3474 | pH Adjustment, | Inorganic |
| | | | | Fungicide, | |
| | | | | Herbicide, | |
| | | | | Microbiocide | |
| Thallium(I) sulfate | 7446-18-6 | 80001 | NA 35 | Rodenticide | Inorganic, Heavy |
| | | | | | metal |
| Titanium dioxide | 13463-67-7 | NA 954 | 2942 | N/A | Inorganic |
| Titanium sulfate | 13825-74-6 | NA 111 | 3476 | N/A | Inorganic |
| Tricopper dichloride | 7076-63-3 | ##### | NA 152 | Microbiocide | Dithiocarbamate, |
| dimethyldithiocarbamate | | | | | Inorganic-Copper |
| Tripotassium phosphate | 7778-53-2 | 76407 | 1055 | pH Adjustment, | Inorganic |
| | | | | Fungicide, | |
| | | | | Herbicide, | |
| | | | | Microbiocide | |
| Trisodium phosphate | 7601-54-9 | 76406 | 1579 | pH Adjustment, | Inorganic |
| | | | | Fungicide, | |
| | | | | Herbicide, | |
| | | | | Microbiocide | |
| Urea | 57-13-6 | 85702 | 662 | Microbiocide, | Inorganic |
| | | | | Fungicide | |
| Urea dihydrogen sulfate | 21351-39-3 | ##### | 2270 | Plant Growth | Inorganic |
| | | | | Regulator, | |
| | | | | Herbicide | |
| Vermiculite | NA 778 | NA 140 | 2983 | N/A | Inorganic |
| Water | NA 557 | NA 101 | 2987 | Solvent | Inorganic |
| Zinc | 7440-66-6 | ##### | 2310 | Herbicide | Inorganic-Zinc |
| Zinc 2,4,5-trichlorophenate | 136-24-3 | 64221 | NA 546 | N/A | Chlorinated Phenol, |
| | | | | | Inorganic-Zinc |
| Zinc 2-ethyl hexoate | 136-53-8 | 41203 | 3509 | N/A | Inorganic-Zinc |
| Zinc 2-pyridinethiol-1-oxide | 13463-41-7 | 88002 | 2128 | Microbiocide | Inorganic-Zinc |
| Zinc 8-quinolinolate | 13978-85-3 | 24005 | NA 196 | Fungicide, | Inorganic-zinc |
| | | | | Microbiocide | |
| Zinc abietate | 6798-76-1 | NA 112 | 3506 | N/A | Inorganic-Zinc |
| Zinc arsenate | 13464-44-3 | 13301 | NA 125 | Herbicide, | Inorganic-Arsenic, |
| | | | | Insecticide, | Heavy metal, |
| | | | | Rodenticide | Inorganic-Zinc |
| Zinc arsenite | 28837-97-0 | 13604 | NA 130 | Herbicide, | Inorganic-Arsenic, |
| | | | | Insecticide, | Inorganic-Zinc, |
| | | | | Rodenticide | Heavy metal |
| Zinc bacitracin | 1405-89-6 | 6309 | NA 87 | Microbiocide | Inorganic-Zinc |
| Zinc carbonate | 3486-35-9 | NA 102 | 3507 | N/A | Inorganic-Zinc |
| Zinc chloride | 7646-85-7 | 87801 | 624 | Microbiocide | Inorganic-Zinc |
| Zinc dehydroabietylammonium | 53404-92-5 | 4211 | NA 75 | Fungicide, | Inorganic-Zinc |
| 2-ethylhexanoate | | | | Microbiocide | |
| Zinc dodecyl benzene sulfonate | 12068-16-5 | NA 112 | 3508 | Microbiocide, | Soap, Inorganic-Zinc |
| | | | | Adjuvant, | |
| | | | | Fungicide, | |
| | | | | Insecticide | |
| Zinc fluosilicate | 16871-71-9 | 75307 | 1019 | Insecticide | Inorganic-Zinc |
| Zinc formate | 557-41-5 | 87802 | NA 838 | N/A | Inorganic-Zinc |
| Zinc hydroxide | 20427-58-1 | 88501 | 3510 | N/A | Inorganic-Zinc |
| Zinc isodecanoate | 30304-30-4 | 41222 | NA 355 | N/A | Inorganic-Zinc |
| Zinc isononanoate | 5398-80-6 | 41212 | NA 353 | N/A | Inorganic-Zinc |
| Zinc isooctanoate | 84082-93-9 | 41205 | NA 351 | N/A | Inorganic-Zinc |
| Zinc mercury chromate | 22323-45-1 | 21004 | NA 170 | Fungicide | Inorganic-Mercury, |
| | | | | | Inorganic- |
| | | | | | chromium(VI), |
| | | | | | Inorganic-Zinc, |
| | | | | | Heavy metal |
| Zinc naphthenate | 12001-85-3 | 88301 | 1111 | Wood | Inorganic-Zinc |
| | | | | Preservative, | |
| | | | | Insecticide, | |
| | | | | Fungicide, Dog | |
| | | | | and Cat | |
| | | | | Repellent | |
| Zinc neodecanoate | 27253-29-8 | 97502 | NA 853 | N/A | Inorganic-Zinc |
| Zinc oxide | 1314-13-2 | 88502 | 666 | Fungicide, | Inorganic-Zinc |
| | | | | Adjuvant | |
| Zinc petroleum (C20-C30) | 68989-17-3 | 88503 | NA 841 | N/A | Inorganic-Zinc |
| sulfonate | | | | | |
| Zinc phenol sulfonate | 127-82-2 | 89002 | NA 842 | N/A | Inorganic-Zinc |
| Zinc phosphide | 1314-84-7 | 88601 | 626 | Rodenticide | Inorganic-Zinc |
| Zinc resinate | 9010-69-9 | 77001 | NA 712 | N/A | Inorganic-Zinc |
| Zinc silicate | NA 1318 | 8103 | NA 176 | Microbiocide | Inorganic-Zinc |
| Zinc stearate | 557-05-1 | 77002 | 3511 | N/A | Inorganic-Zinc |
| Zinc sulfate | 7733-02-0 | 89001 | 667 | Microbiocide, | Inorganic-Zinc |
| | | | | Herbicide | |
| Zinc sulfate, anhydrous | 7733-02-0 | 89001 | 1852 | Microbiocide, | Inorganic-Zinc |
| | | | | Herbicide | |
| Zinc sulfate, basic | 68813-94-5 | 89101 | NA 843 | N/A | Inorganic-Zinc |
| Zinc sulfate, monohydrate | 7446-19-7 | ##### | 2995 | Herbicide | Inorganic-Zinc |
| Zinc trichlorophenate | 30143-22-7 | 64213 | NA 543 | Microbiocide | Chlorinated Phenol, |
| | | | | | Inorganic-Zinc |
| Zineb | 12122-67-7 | 14506 | 627 | Fungicide | Dithiocarbamate, |
| | | | | | Inorganic-Zinc |
| Zineb-ethylene thiuram | NA 1465 | NA 217 | NA 216 | Fungicide | Dithiocarbamate, |
| disulfide adduct | | | | | Inorganic-Zinc |
| Ziram | 137-30-4 | 34805 | 629 | Fungicide, | Dithiocarbamate, |
| | | | | Microbiocide, | Inorganic-Zinc |
| | | | | Dog and Cat | |
| | | | | Repellent | |
| Ziram, cyclohexylamine | 16509-79-8 | 34806 | 1328 | Dog and Cat | Dithiocarbamate, |
| complex | | | | Repellent, | Inorganic-Zinc |
| | | | | Fungicide | |
| (3-Ethoxypropyl)mercury | 6012-84-6 | ##### | NA 147 | Fungicide | Organomercury, |
| bromide | | | | | Heavy metal |
| (3-Hydroxy-2- | 69653-69-6 | ##### | NA 153 | N/A | Organomercury, |
| methoxypropyl)mercuric | | | | | Heavy metal |
| acetate | | | | | |
| 10,10′-Oxybisphenoxyarsine | 58-36-6 | 12601 | 1402 | Fungicide, | Organoarsenic, |
| | | | | Microbiocide | Heavy metal |
| 2-(acetoxymercuri)ethanol | 4665-55-8 | 41501 | NA 356 | N/A | Organomercury, |
| | | | | | Heavy metal |
| 3-(Hydroxymercuri)-4-nitro-o- | 53404-55-0 | 52604 | NA 447 | Microbiocide | Organomercury, |
| phenol, sodium salt | | | | | Heavy metal |
| Ammonium arsenate | 7784-44-3 | 13601 | 2380 | Herbicide, | Inorganic-arsenic, |
| | | | | Insecticide, | Heavy metal |
| | | | | Rodenticide | |
| Ammonium dichromate | 2149701 | 68305 | NA 607 | Wood | Inorganic- |
| | | | | Preservative | Chromium(VI), |
| | | | | | Heavy metal |
| Anilinocadmium dilactate | 19651-91-3 | 64601 | NA 548 | N/A | Inorganic-Cadmium, |
| | | | | | Heavy metal |
| Arsenic | 7440-38-2 | NA 121 | 710 | Herbicide, | Inorganic-arsenic, |
| | | | | Insecticide, | Heavy metal |
| | | | | Rodenticide | |
| Arsenic acid | 7778-39-4 | 6801 | 40 | Herbicide, | Inorganic-arsenic, |
| | | | | Insecticide, | Heavy metal |
| | | | | Rodenticide | |
| Arsenic disulfide | 56320-22-0 | 6901 | NA 98 | Herbicide, | Inorganic-arsenic, |
| | | | | Insecticide, | Heavy metal |
| | | | | Rodenticide | |
| Arsenic pentoxide | 1303-28-2 | 6802 | 631 | Fungicide, | Inorganic-arsenic, |
| | | | | Insecticide, | Heavy metal |
| | | | | Rodenticide, | |
| | | | | Herbicide | |
| Arsenic sulfide | 12612-21-4 | 6901 | NA 99 | Herbicide, | Inorganic-arsenic, |
| | | | | Insecticide, | Heavy metal |
| | | | | Rodenticide | |
| Arsenic trioxide | 1327-53-3 | 7001 | 42 | Herbicide, | Inorganic-arsenic, |
| | | | | Insecticide, | Heavy metal |
| | | | | Rodenticide | |
| Arsenic trisulfide | 1303-33-9 | 6901 | NA 97 | Herbicide, | Inorganic-arsenic, |
| | | | | Insecticide, | Heavy metal |
| | | | | Rodenticide | |
| Arsenosobenzene | 637-03-6 | 7101 | NA 100 | N/A | Organoarsenic, |
| | | | | | Heavy metal |
| Arsonic acid, (4-aminophenyl)- | 98-50-0 | ##### | NA 109 | N/A | Organoarsenic, |
| | | | | | Heavy metal |
| Arsonic acid, ammonium salt | 58829-95-1 | ##### | NA 105 | Herbicide, | Inorganic-arsenic, |
| | | | | Insecticide, | Heavy metal |
| | | | | Wood | |
| | | | | Preservative | |
| Azocyclotin | 41083-11-8 | ##### | 2408 | Insecticide | Organotin, Heavy |
| | | | | | metal |
| Basic bismuth gallate | 99-26-3 | 98601 | NA 860 | N/A | Heavy metal |
| Basic copper arsenate | 16102-92-4 | ##### | NA 132 | Herbicide, | Inorganic-arsenic, |
| | | | | Insecticide, | Heavy metal, |
| | | | | Rodenticide | Inorganic-Copper |
| Basic lead silicate | 53466-66-3 | 48003 | NA 417 | N/A | Inorganic-Lead, |
| | | | | | Heavy metal |
| Bis (tributyltin) adipate | 7437-35-6 | 83117 | 1990 | Antifoulant, | Organotin, Heavy |
| | | | | Microbiocide | metal |
| Bis (tributyltin) sulfide | 4808-30-4 | 83113 | 1886 | Antifoulant, | Organotin, Heavy |
| | | | | Microbiocide | metal |
| Bis (tributyltin) sulfone | NA 970 | NA 476 | 1665 | Antifoulant, | Organotin, Heavy |
| | | | | Microbiocide | metal |
| Bis(tributyltin) | 12379-54-3 | 83101 | NA 824 | Antifoulant, | Organotin, Heavy |
| dodecenylsuccinate | | | | Microbiocide | metal |
| Bis(tributyltin) salicylate | 22330-14-9 | 83102 | 5074 | Antifoulant, | Organotin, Heavy |
| | | | | Microbiocide | metal |
| Bis(tributyltin) succinate | 4644-96-6 | 83103 | 74 | Antifoulant, | Organotin, Heavy |
| | | | | Microbiocide | metal |
| Bis(tributyltin) sulfosalicylate | 4419-22-1 | 83104 | NA 825 | Antifoulant, | Organotin, Heavy |
| | | | | Microbiocide | metal |
| Bis(tripropyltin) oxide | 1067-29-4 | 83201 | NA 829 | Microbiocide, | Organotin, Heavy |
| | | | | Fungicide | metal |
| Cacodylic acid | 75-60-5 | 12501 | 32 | Herbicide, | Organoarsenic, |
| | | | | Defoliant | Heavy metal |
| Cadmium | 7440-43-9 | NA 460 | 2438 | N/A | Inorganic-Cadmium, |
| | | | | | Heavy metal |
| Cadmium carbonate | 513-78-0 | 12901 | 93 | N/A | Inorganic-Cadmium, |
| | | | | | Heavy metal |
| Cadmium chloride | 10108-64-2 | 12902 | 94 | N/A | Inorganic-Cadmium, |
| | | | | | Heavy metal |
| Cadmium cocoate | 72869-63-7 | NA 459 | 3085 | N/A | Inorganic-Cadmium, |
| | | | | | Heavy metal |
| Cadmium oxide | 1306-19-0 | ##### | NA 130 | N/A | Inorganic-Cadmium, |
| | | | | | Heavy metal |
| Cadmium perborate | NA 969 | NA 461 | 3086 | N/A | Inorganic-Cadmium, |
| | | | | | Heavy metal |
| Cadmium sebacate | 939499 | 12903 | 699 | N/A | Inorganic-Cadmium, |
| | | | | | Heavy metal |
| Cadmium succinate | 141-00-4 | 12904 | 92 | N/A | Inorganic-Cadmium, |
| | | | | | Heavy metal |
| Cadmium sulfate | 10124-36-4 | 12905 | NA 123 | N/A | Inorganic-Cadmium, |
| | | | | | Heavy metal |
| Cadmium yellow pigments | NA 967 | NA 448 | 2439 | N/A | Inorganic-Cadmium, |
| | | | | | Heavy metal |
| Calcium acid methanearsonate | 5902-95-4 | 13806 | 101 | Herbicide, | Organoarsenic, |
| | | | | Defoliant | Heavy metal |
| Calcium arsenate | 7778-44-1 | 13501 | 96 | Herbicide, | Inorganic-arsenic, |
| | | | | Insecticide, | Heavy metal |
| | | | | Rodenticide, | |
| | | | | Molluscicide | |
| Calcium arsenite | 53404-59-4 | 13602 | NA 129 | Herbicide, | Inorganic-arsenic, |
| | | | | Insecticide, | Heavy metal |
| | | | | Rodenticide | |
| Calcium methanearsonate | 6423-72-9 | 13809 | NA 136 | Herbicide, | Organoarsenic, |
| | | | | Defoliant | Heavy metal |
| Calcium propanearsonate | 126-94-3 | 13801 | NA 133 | Herbicide, | Organoarsenic, |
| | | | | Defoliant | Heavy metal |
| Calomel | 10112-91-1 | 52201 | 100 | Fungicide | Inorganic-Mercury, |
| | | | | | Heavy metal |
| Chloromethoxy propyl | 1319-86-4 | 18401 | 887 | N/A | Organomercury, |
| mercuric acetamide | | | | | Heavy metal |
| Chromated zinc chloride | NA 1026 | 87801 | 1187 | Insecticide, | Inorganic- |
| | | | | Herbicide, | Chromium(VI), |
| | | | | Microbiocide | Inorganic-Zinc, |
| | | | | | Heavy metal |
| Chromic acetate | 1066-30-4 | 21102 | NA 172 | Wood | Inorganic- |
| | | | | Preservative | Chromium(VI), |
| | | | | | Heavy metal |
| Chromic acid | 7738-94-5 | 21101 | 1188 | Wood | Inorganic- |
| | | | | Preservative | Chromium(VI), |
| | | | | | Heavy metal |
| Chromic oxide | 1308-38-9 | 21103 | NA 173 | Wood | Inorganic- |
| | | | | Preservative | Chromium(VI), |
| | | | | | Heavy metal |
| Copper arsenate | 10103-61-4 | 22801 | NA 178 | Herbicide, | Inorganic-arsenic, |
| | | | | Insecticide, | Heavy metal, |
| | | | | Rodenticide | Inorganic-Copper |
| Copper arsenite | 10290-12-7 | 22401 | NA 177 | Herbicide, | Inorganic-arsenic, |
| | | | | Insecticide, | Heavy metal, |
| | | | | Rodenticide | Inorganic-Copper |
| Copper-zinc chromate complex | 1344-74-7 | 21003 | 163 | Fungicide, | Inorganic- |
| | | | | Wood | Chromium(VI), |
| | | | | Preservative | Inorganic-Zinc, |
| | | | | | Heavy metal, |
| | | | | | Inorganic-Copper |
| Crag turf fungicide 531 | 12001-20-6 | 21006 | NA 171 | Fungicide | Inorganic-Cadmium, |
| (Cd—Cu—Ca—Zn-Chromate) | | | | | Inorganic- |
| | | | | | Chromium(VI), |
| | | | | | Inorganic-Zinc, |
| | | | | | Heavy metal |
| Cupric acetoarsenite | 12002-03-8 | 22601 | 2485 | Wood | Inorganic-arsenic, |
| | | | | Preservative | Heavy metal, |
| | | | | | Inorganic-Copper |
| Cyhexatin | 13121-70-5 | ##### | 1638 | Insecticide | Organotin, Heavy |
| | | | | | metal |
| Decafentin | 15652-38-7 | ##### | NA 132 | N/A | Organotin, Heavy |
| | | | | | metal |
| Di(phenylmercuri) ammonium | 18467-88-4 | 66002 | NA 551 | N/A | Organomercury, |
| propionate | | | | | Heavy metal |
| Di(phenylmercuric) dodecenyl | 27236-65-3 | 66001 | 501 | Microbiocide, | Organomercury, |
| succinate | | | | Fungicide | Heavy metal |
| Dibutyltin dichloride | 683-18-1 | 83123 | NA 828 | N/A | Organotin, Heavy |
| | | | | | metal |
| Dodecyl ammonium | 53404-47-0 | 13805 | 857 | Herbicide, | Organoarsenic, |
| methanearsonate | | | | Defoliant | Heavy metal |
| DSMA | 144-21-8 | 13802 | 251 | Herbicide, | Organoarsenic, |
| | | | | Defoliant | Heavy metal |
| Ethylene oxide condensate of | 56573-85-4 | NA 126 | 1864 | Antifoulant, | Organotin, Heavy |
| abietylamine, tributyltin | | | | Microbiocide | metal |
| chloride | | | | | |
| Ethylmercuric phosphate | 2235-25-8 | 41505 | 357 | N/A | Organomercury, |
| | | | | | Heavy metal |
| Ethylmercurichlorendiimide | 2597-93-5 | 45301 | NA 383 | N/A | Organomercury, |
| | | | | | Heavy metal |
| Ethylmercurithiosalicylic acid | 148-61-8 | 78902 | NA 724 | Fungicide | Organomercury, |
| | | | | | Heavy metal |
| Ethylmercury 2,3- | 2597-92-4 | 41507 | NA 361 | Fungicide | Organomercury, |
| dihydroxypropylmercaptide | | | | | Heavy metal |
| Ethylmercury acetate | 109-62-6 | 41502 | NA 357 | Fungicide | Organomercury, |
| | | | | | Heavy metal |
| Ethylmercury bromide | 107-26-6 | ##### | NA 149 | Fungicide | Organomercury, |
| | | | | | Heavy metal |
| Ethylmercury chloride | 107-27-7 | 41503 | NA 358 | Fungicide | Organomercury, |
| | | | | | Heavy metal |
| Ethylmercury | 22232-28-6 | 41504 | NA 359 | Microbiocide | Organomercury, |
| pentachlorophenate | | | | | Chlorinated Phenol, |
| | | | | | Heavy metal |
| Fatty acid* - silver complex | 24927-67-1 | 72505 | NA 675 | Microbiocide, | Inorganic-Silver, |
| *(100% C8) | | | | Fungicide, | Heavy metal |
| | | | | Herbicide | |
| Fenbutatin-oxide | 13356-08-6 | ##### | 1876 | Insecticide | Organotin, Heavy |
| | | | | | metal |
| Fentin hydroxide | 76-87-9 | 83601 | 599 | Fungicide, | Organotin, Heavy |
| | | | | Molluscicide, | metal |
| | | | | Herbicide | |
| Hydroxymercuri-o-nitrophenol | 30284-78-7 | 52601 | 2606 | Microbiocide | Organomercury, |
| | | | | | Heavy metal |
| Hydroxymercuri-o-nitrophenol, | 1300-34-1 | 52602 | NA 445 | Microbiocide | Organomercury, |
| sodium salt | | | | | Heavy metal |
| Hydroxymercurichlorophenols | 53466-93-6 | 46002 | NA 391 | Microbiocide | Organomercury, |
| | | | | | Heavy metal |
| Hydroxymercuriphenyl | 53466-95-8 | 46003 | NA 392 | Microbiocide | Organomercury, |
| ammonium triethanolamine | | | | | Heavy metal |
| Hydroxymercury cresol | 12379-66-7 | 46001 | NA 390 | Microbiocide | Inorganic-Mercury, |
| | | | | | Heavy metal, Phenols |
| Lactoxymercuriphenyl | 53466-98-1 | 51901 | NA 429 | Microbiocide | Organomercury, |
| ammonium lactate | | | | | Heavy metal |
| Lauric acid, barium cadmium | 15337-60-7 | NA 118 | 3676 | Adjuvant | Inorganic-Cadmium, |
| salt | | | | | Heavy metal |
| Lead | 7439-92-1 | NA 134 | 2638 | N/A | Inorganic-Lead, |
| | | | | | Heavy metal |
| Lead acetate | 301-04-2 | 48001 | NA 415 | N/A | Inorganic-Lead, |
| | | | | | Heavy metal |
| Lead arsenate | 7784-40-9 | 13503 | 353 | Herbicide, | Inorganic-arsenic, |
| | | | | Insecticide, | Inorganic-lead, |
| | | | | Rodenticide | Heavy metal |
| Lead arsenate, basic | 1327-31-7 | 13502 | 354 | Herbicide, | Inorganic-arsenic, |
| | | | | Insecticide, | Inorganic-lead, |
| | | | | Rodenticide | Heavy metal |
| Lead metasilicate | NA 938 | NA 138 | 1106 | N/A | Inorganic-Lead, |
| | | | | | Heavy metal |
| Lead monoxide | 10190-55-3 | NA 123 | 1271 | N/A | Inorganic-Lead, |
| | | | | | Heavy metal |
| Lead naphthenate | 61790-14-5 | 48004 | 3296 | N/A | Inorganic-Lead, |
| | | | | | Heavy metal |
| Litharge | 1317-36-8 | 48002 | NA 416 | N/A | Inorganic-Lead, |
| | | | | | Heavy metal |
| Magnesium arsenate | 10103-50-1 | 13504 | NA 126 | Herbicide, | Inorganic-arsenic, |
| | | | | Insecticide, | Heavy metal |
| | | | | Rodenticide | |
| Manganese arsenate | 7784-38-5 | 13506 | NA 127 | Herbicide, | Inorganic-arsenic, |
| | | | | Insecticide, | Heavy metal |
| | | | | Rodenticide | |
| Mercuric acetate | 1600-27-7 | 52104 | NA 441 | N/A | Inorganic-Mercury, |
| | | | | | Heavy metal |
| Mercuric chloride | 7487-94-7 | 52001 | 372 | Fungicide | Inorganic-Mercury, |
| | | | | | Heavy metal |
| Mercuric dimethyl | 15415-64-2 | 34808 | 709 | Microbiocide | Inorganic-Mercury, |
| dithiocarbamate | | | | | Heavy metal, |
| | | | | | Dithiocarbamate |
| Mercuric iodide | 7774-29-0 | 52003 | NA 438 | N/A | Inorganic-Mercury, |
| | | | | | Heavy metal |
| Mercuric oleate | 1191-80-6 | 52105 | 3276 | N/A | Inorganic-Mercury, |
| | | | | | Heavy metal |
| Mercuric oxide | 21908-53-2 | 52102 | 955 | Fungicide | Inorganic-Mercury, |
| | | | | | Heavy metal |
| Mercury | 7439-97-6 | 52301 | NA 26 | N/A | Inorganic-Mercury, |
| | | | | | Heavy metal |
| Mercury naphthenate | 1336-96-5 | 52101 | NA 439 | Wood | Inorganic-Mercury, |
| | | | | Preservative, | Heavy metal |
| | | | | Insecticide, | |
| | | | | Fungicide | |
| Mercury pentanedione | 14024-55-6 | 52103 | NA 440 | N/A | Inorganic-Mercury, |
| | | | | | Heavy metal |
| Mercury phenate | 588-66-9 | 52106 | NA 442 | N/A | Inorganic-Mercury, |
| | | | | | Heavy metal |
| Metallic silver | 7440-22-4 | 72501 | 2125 | Microbiocide, | Inorganic-Silver, |
| | | | | Fungicide, | Heavy metal |
| | | | | Herbicide | |
| Methane arsonic acid (MAA) | 124-58-3 | ##### | NA 27 | N/A | Organoarsenic, |
| | | | | | Heavy metal |
| Methoxyethylmercury acetate | 151-38-2 | 41508 | NA 362 | Fungicide | Organomercury, |
| | | | | | Heavy metal |
| Methylenebis(thiocyanate) with | 76397-81-4 | 81408 | NA 785 | Antifoulant, | Organotin, Heavy |
| TBTO | | | | Microbiocide | metal |
| Methylmercurichlorendimide | 5902-79-4 | 45302 | NA 384 | N/A | Organomercury, |
| | | | | | Heavy metal |
| Methylmercury 2,3- | 2597-95-7 | 51905 | NA 433 | Fungicide | Organomercury, |
| dihydroxypropylmercaptide | | | | | Heavy metal |
| Methylmercury 8- | 86-85-1 | 51902 | NA 430 | Fungicide | Organomercury, |
| hydroxyquinolinate | | | | | Heavy metal |
| Methylmercury acetate | 108-07-6 | 51903 | NA 431 | Fungicide | Organomercury, |
| | | | | | Heavy metal |
| Methylmercury benzoate | 3626-13-9 | 51904 | NA 432 | Fungicide | Organomercury, |
| | | | | | Heavy metal |
| Methylmercury dicyano | 502-39-6 | 51909 | 454 | Fungicide | Organomercury, |
| diamide | | | | | Heavy metal |
| Methylmercury hydroxide | 1184-57-2 | 51906 | NA 434 | Fungicide | Organomercury, |
| | | | | | Heavy metal |
| Methylmercury nitrile | 2597-97-9 | 51907 | NA 435 | Fungicide | Organomercury, |
| | | | | | Heavy metal |
| Methylmercury propionate | 1460883 | 51908 | NA 436 | Fungicide | Organomercury, |
| | | | | | Heavy metal |
| Monoammonium | 2321-53-1 | 13808 | NA 135 | Herbicide, | Organoarsenic, |
| methanearsonate | | | | Defoliant | Heavy metal |
| Monotributyltin salicylate | 4342-30-7 | 83122 | NA 827 | Antifoulant, | Organotin, Heavy |
| | | | | Microbiocide | metal |
| MSMA | 2163-80-6 | 13803 | 34 | Herbicide, | Organoarsenic, |
| | | | | Defoliant | Heavy metal |
| N-(Ethylmercury)-p- | 517-16-8 | 41506 | NA 360 | Fungicide | Organomercury, |
| toluenesulfonanilide | | | | | Heavy metal |
| N-(Phenylmercuri) urea | 2279-64-3 | ##### | NA 160 | Herbicide | Organomercury, |
| | | | | | Heavy metal |
| N- | 5980-82-5 | 66009 | NA 556 | Fungicide | Organomercury, |
| (Phenylmercuri)ethylenediamine | | | | | Heavy metal |
| o-(Chloromercuri)phenol | 90-03-9 | ##### | NA 135 | Microbiocide | Organomercury, |
| | | | | | Heavy metal |
| o-(Hydroxymercuri)benzoic | 5722-59-8 | 52603 | NA 446 | Microbiocide | Organomercury, |
| acid, cyclic anhydride | | | | | Heavy metal |
| Octylammonium | 6379-37-9 | 13804 | 27 | Herbicide, | Organoarsenic, |
| methanearsonate | | | | Defoliant | Heavy metal |
| p-tert- | 53433-01-5 | 52002 | NA 437 | Microbiocide | Inorganic-Mercury, |
| Octylphenoxyethoxyethyl | | | | | Heavy metal |
| dimethyl benzyl ammonium | | | | | |
| mercuric chloride | | | | | |
| Paris green | 12002-03-8 | 22601 | 460 | Wood | Inorganic-Arsenic, |
| | | | | Preservative | Inorganic-Copper, |
| | | | | | Heavy metal |
| Phenarsazine chloride | 578-94-9 | 63901 | 1342 | N/A | Organoarsenic, |
| | | | | | Heavy metal |
| Phenarsazine oxide | 4095-45-8 | 12602 | NA 122 | N/A | Organoarsenic, |
| | | | | | Heavy metal |
| Phenylmercuriammonium | 53404-68-5 | 66023 | NA 567 | Fungicide | Organomercury, |
| propionate | | | | | Heavy metal |
| Phenylmercuric 2- | 13302-00-6 | 66024 | NA 568 | Fungicide, | Organomercury, |
| ethylhexanoate | | | | Microbiocide | Heavy metal |
| Phenylmercuric borate | 6273-99-0 | 66005 | NA 552 | Fungicide, | Organomercury, |
| | | | | Microbiocide | Heavy metal |
| Phenylmercuric carbonate | 53404-69-6 | 66006 | NA 553 | Fungicide, | Organomercury, |
| | | | | Microbiocide | Heavy metal |
| Phenylmercuric chloride | 100-56-1 | 66007 | NA 554 | Fungicide, | Organomercury, |
| | | | | Microbiocide | Heavy metal |
| Phenylmercuric | 32407-99-1 | 66008 | NA 555 | Microbiocide, | Organomercury, |
| dimethyldithiocarbamate | | | | Fungicide | Dithiocarbamate, |
| | | | | | Heavy metal |
| Phenylmercuric formamide | 22894-47-9 | 66010 | NA 557 | Fungicide, | Organomercury, |
| | | | | Microbiocide, | Heavy metal |
| | | | | Herbicide | |
| Phenylmercuric hydroxide | 100-57-2 | 66011 | NA 558 | Fungicide, | Organomercury, |
| | | | | Microbiocide, | Heavy metal |
| | | | | Herbicide | |
| Phenylmercuric lactate | 122-64-5 | 66012 | 1535 | Microbiocide, | Organomercury, |
| | | | | Fungicide, | Heavy metal |
| | | | | Herbicide | |
| Phenylmercuric | 5822-97-9 | 66013 | NA 559 | Fungicide, | Organomercury, |
| monoethanolammonium | | | | Microbiocide, | Heavy metal |
| acetate | | | | Herbicide | |
| Phenylmercuric | 53404-70-9 | 66014 | NA 560 | Fungicide, | Organomercury, |
| monoethanolammonium lactate | | | | Microbiocide, | Heavy metal |
| | | | | Herbicide | |
| Phenylmercuric naphthenate | 31632-68-5 | 66015 | NA 561 | Wood | Organomercury, |
| | | | | Preservative, | Heavy metal |
| | | | | Insecticide, | |
| | | | | Fungicide | |
| Phenylmercuric nitrate | 55-68-5 | 66016 | 477 | Microbiocide, | Organomercury, |
| | | | | Fungicide, | Heavy metal |
| | | | | Herbicide | |
| Phenylmercuric octanoate | 13864-38-5 | 66025 | NA 569 | Fungicide, | Organomercury, |
| | | | | Microbiocide, | Heavy metal |
| | | | | Herbicide | |
| Phenylmercuric oleate | 104-60-9 | 66022 | 853 | Microbiocide, | Organomercury, |
| | | | | Fungicide, | Heavy metal |
| | | | | Herbicide | |
| Phenylmercuric propionate | 103-27-5 | 66018 | NA 563 | Fungicide, | Organomercury, |
| | | | | Microbiocide, | Heavy metal |
| | | | | Herbicide | |
| Phenylmercuric salicylate | 28086-13-7 | 66019 | NA 564 | Fungicide, | Organomercury, |
| | | | | Microbiocide, | Heavy metal |
| | | | | Herbicide | |
| Phenylmercuric thiocyanate | 16751-55-6 | 66020 | NA 565 | Microbiocide, | Organomercury, |
| | | | | Fungicide, | Heavy metal |
| | | | | Herbicide | |
| Phenylmercuritriethanolammonium | 23319-66-6 | 66021 | NA 566 | Microbiocide, | Organomercury, |
| lactate | | | | Fungicide, | Heavy metal |
| | | | | Herbicide | |
| Phenylmercury 8- | 14354-56-4 | 66017 | NA 562 | Fungicide, | Organomercury, |
| hydroxyquinolinolate | | | | Microbiocide, | Heavy metal |
| | | | | Herbicide | |
| Phenylmercury benzoate | 94-43-9 | 51900 | NA 428 | Fungicide, | Organomercury, |
| | | | | Microbiocide, | Heavy metal |
| | | | | Herbicide | |
| PMA | 62-38-4 | 66003 | 491 | Fungicide, | Organomercury, |
| | | | | Microbiocide, | Heavy metal |
| | | | | Herbicide | |
| PMA, other related | NA 1106 | NA 164 | 90491 | Fungicide, | Organomercury, |
| | | | | Herbicide, | Heavy metal |
| | | | | Microbiocide | |
| PMAA | 53404-67-4 | 66004 | 492 | Fungicide, | Organomercury, |
| | | | | Microbiocide | Heavy metal |
| Poly (methylmethacrylate-co- | 26354-18-7 | 83119 | 5075 | Antifoulant, | Organotin, Heavy |
| tributyltin methacrylate) | | | | Microbiocide | metal |
| Potassium arsenite | 10124-50-2 | 13605 | NA 131 | Herbicide, | Inorganic-arsenic, |
| | | | | Insecticide, | Heavy metal |
| | | | | Rodenticide | |
| Potassium arsenite | 13464-35-2 | 13605 | NA 132 | Herbicide, | Inorganic-arsenic, |
| | | | | Insecticide, | Heavy metal |
| | | | | Rodenticide | |
| Potassium chromate | 7789-00-6 | 68301 | 700 | Wood | Inorganic- |
| | | | | Preservative | Chromium(VI), |
| | | | | | Heavy metal |
| Potassium hexafluoroarsenate | 17029-22-0 | ##### | NA 160 | N/A | Inorganic-arsenic, |
| | | | | | Heavy metal |
| Potassium mercuric iodide | 7783-33-7 | 52107 | NA 443 | N/A | Inorganic-Mercury, |
| | | | | | Heavy metal |
| Pyridylmercuric acetate | 1334-77-6 | 69501 | NA 661 | N/A | Organomercury, |
| | | | | | Heavy metal |
| Pyridylmercuric chloride | 1334-75-4 | 69502 | NA 662 | N/A | Organomercury, |
| | | | | | Heavy metal |
| Silver acetate | 563-63-3 | 72507 | NA 676 | Microbiocide, | Inorganic-Silver, |
| | | | | Fungicide, | Heavy metal |
| | | | | Herbicide | |
| Silver carbonate | 534-16-7 | 72509 | NA 678 | Microbiocide, | Inorganic-Silver, |
| | | | | Fungicide, | Heavy metal |
| | | | | Herbicide | |
| Silver chloride | 7783-90-6 | 72506 | 2324 | Microbiocide, | Inorganic-Silver, |
| | | | | Fungicide, | Heavy metal |
| | | | | Herbicide | |
| Silver copper zeolite | 130328-19-7 | ##### | NA 110 | Microbiocide | Inorganic-Silver, |
| | | | | | Heavy metal, |
| | | | | | Inorganic-Copper |
| Silver fluoride | 7775-41-9 | 72502 | NA 674 | Microbiocide, | Inorganic-Silver, |
| | | | | Fungicide, | Heavy metal |
| | | | | Herbicide | |
| Silver iodide, colloidal | 7783-96-2 | NA 124 | 1556 | Microbiocide, | Inorganic-Silver, |
| | | | | Fungicide, | Heavy metal |
| | | | | Herbicide | |
| Silver nitrate | 7761-88-8 | 72503 | 2856 | Microbiocide, | Inorganic-Silver, |
| | | | | Fungicide, | Heavy metal |
| | | | | Herbicide, | |
| | | | | Plant Growth | |
| | | | | Regulator | |
| Silver orthophosphate | 7784-09-0 | 72510 | NA 679 | Microbiocide, | Inorganic-Silver, |
| (Ag3PO4) | | | | Fungicide, | Heavy metal |
| | | | | Herbicide | |
| Silver oxide (Ag4O4) | 1301-96-8 | ##### | NA 111 | Microbiocide, | Inorganic-Silver, |
| | | | | Fungicide, | Heavy metal |
| | | | | Herbicide | |
| Silver oxide (Ag4O4) | 155645-89-9 | ##### | NA 111 | Microbiocide, | Inorganic-Silver, |
| | | | | Fungicide, | Heavy metal |
| | | | | Herbicide | |
| Silver sodium hydrogen | NA 1166 | 72560 | NA 164 | Microbiocide | Inorganic-Silver, |
| zirconium phosphate | | | | | Heavy metal |
| (Ag0.18Na0.57H0.25Zr2(PO4)3) | | | | | |
| Silver thiocyanate | 1701-93-5 | 68203 | NA 606 | Microbiocide, | Inorganic-Silver, |
| | | | | Fungicide, | Heavy metal |
| | | | | Herbicide | |
| Silver thiuronium acrylate copolymer | 53404-00-5 | 72701 | NA 681 | Microbiocide, | Inorganic-Silver, |
| | | | | Fungicide, | Heavy metal |
| | | | | Herbicide | |
| Silver zeolite | 130328-18-6 | ##### | NA 125 | Microbiocide | Inorganic-Silver, |
| | | | | | Heavy metal |
| Silver zinc zeolite | 130328-20-0 | ##### | NA 111 | Microbiocide | Inorganic-Silver, |
| | | | | | Inorganic-Zinc, |
| | | | | | Heavy metal |
| Silver, ionic | 14701-21-4 | NA 169 | 5150 | Microbiocide, | Inorganic-Silver, |
| | | | | Fungicide, | Heavy metal |
| | | | | Herbicide | |
| Sodium arsenate | 13464-38-5 | 13505 | 283 | Herbicide, | Inorganic-arsenic, |
| | | | | Insecticide, | Heavy metal |
| | | | | Rodenticide | |
| Sodium arsenite | 7784-46-5 | 13603 | 534 | Herbicide, | Inorganic-arsenic, |
| | | | | Insecticide, | Heavy metal |
| | | | | Rodenticide, | |
| | | | | Fungicide | |
| Sodium cacodylate | 124-65-2 | 12502 | 1673 | Herbicide, | Organoarsenic, |
| | | | | Defoliant, | Heavy metal |
| | | | | Rodenticide | |
| Sodium chromate | 2144646 | 68303 | 1241 | Wood | Inorganic- |
| | | | | Preservative | Chromium(VI), |
| | | | | | Heavy metal |
| Sodium dichromate | 10588-01-9 | 68304 | 3395 | Wood | Inorganic- |
| | | | | Preservative | Chromium(VI), |
| | | | | | Heavy metal |
| Sodium | 54-64-8 | 78901 | NA 723 | N/A | Organomercury, |
| ethylmercurithiosalicylate | | | | | Heavy metal |
| Sodium pyroarsenate | 13464-42-1 | 13401 | 2864 | Wood | Inorganic-arsenic, |
| | | | | Preservative, | Heavy metal |
| | | | | Fungicide | |
| Sodium silver thiosulfate | NA 1398 | NA 197 | NA 196 | Herbicide | Inorganic-Silver, |
| | | | | | Heavy metal |
| Sodium thioarsenate | 17367-56-5 | 13507 | NA 128 | N/A | Inorganic-arsenic, |
| (Na3AsO3S) | | | | | Heavy metal |
| Thallium(I) sulfate | 7446-18-6 | 80001 | NA 35 | Rodenticide | Inorganic, Heavy |
| | | | | | metal |
| Tolylmercuric acetate | 1300-78-3 | ##### | NA 150 | Fungicide | Organomercury, |
| | | | | | Heavy metal |
| Tri-n-butyltin maleate | 14275-57-1 | 83118 | 2185 | Antifoulant, | Organotin, Heavy |
| | | | | Microbiocide | metal |
| Tributyltin acetate | 56-36-0 | 83105 | 2097 | Antifoulant, | Organotin, Heavy |
| | | | | Microbiocide | metal |
| Tributyltin acrylate | 13331-52-7 | 83121 | NA 826 | Antifoulant, | Organotin, Heavy |
| | | | | Microbiocide | metal |
| Tributyltin benzoate | 4342-36-3 | 83106 | 1114 | Antifoulant, | Organotin, Heavy |
| | | | | Microbiocide | metal |
| Tributyltin chloride | 1461-22-9 | 83107 | 1891 | Antifoulant, | Organotin, Heavy |
| | | | | Microbiocide | metal |
| Tributyltin chloride complex of | 56573-85-4 | 83108 | 1412 | Antifoulant, | Organotin, Heavy |
| ethylene oxide condensate of | | | | Microbiocide | metal |
| abietylamine | | | | | |
| Tributyltin chloride, | NA 1012 | NA 970 | 938 | Antifoulant, | Organotin, Heavy |
| myristylamine salt | | | | Microbiocide | metal |
| Tributyltin fluoride | 29131 | 83112 | 1030 | Antifoulant, | Organotin, Heavy |
| | | | | Microbiocide, | metal |
| | | | | Fungicide | |
| Tributyltin isopropyl succinate | 53404-82-3 | 83115 | 1115 | Antifoulant, | Organotin, Heavy |
| | | | | Microbiocide | metal |
| Tributyltin linoleate | 24124-25-2 | 83109 | 1116 | Antifoulant, | Organotin, Heavy |
| | | | | Microbiocide | metal |
| Tributyltin methacrylate | 2155-70-6 | 83120 | 2179 | Antifoulant, | Organotin, Heavy |
| | | | | Microbiocide | metal |
| Tributyltin monopropylene | 53466-85-6 | 83110 | 1985 | Antifoulant, | Organotin, Heavy |
| glycol maleate | | | | Microbiocide | metal |
| Tributyltin neodecanoate | 28801-69-6 | 83111 | 1035 | Antifoulant, | Organotin, Heavy |
| | | | | Microbiocide | metal |
| Tributyltin oxide | 56-35-9 | 83001 | 569 | Antifoulant, | Organotin, Heavy |
| | | | | Microbiocide, | metal |
| | | | | Molluscicide, | |
| | | | | Fungicide | |
| Tributyltin resinate | 13387-91-2 | 83114 | 1732 | Antifoulant, | Organotin, Heavy |
| | | | | Microbiocide | metal |
| Tributyltin sulfide | 4808-30-4 | 83113 | 1352 | Antifoulant, | Organotin, Heavy |
| | | | | Microbiocide | metal |
| Triethanolamine cacodylate | 69484-14-6 | 12503 | NA 121 | Herbicide, | Organoarsenic, |
| | | | | Defoliant | Heavy metal |
| Triethanolamine | 5902-97-6 | 13807 | NA 134 | Herbicide, | Organoarsenic, |
| methanearsonate | | | | Defoliant | Heavy metal |
| Trimethyltin chloride | 1066-45-1 | ##### | NA 161 | N/A | Organotin, Heavy |
| | | | | | metal |
| Triphenylbismuthine | 603-33-8 | 82801 | NA 821 | N/A | Heavy metal |
| Triphenylbismuthine dichloride | 594-30-9 | 82802 | NA 822 | N/A | Heavy metal |
| Triphenyltin acetate | 900-95-8 | ##### | NA 153 | Fungicide, | Organotin, Heavy |
| | | | | Herbicide | metal |
| Triphenyltin chloride | 639-58-7 | ##### | NA 153 | N/A | Organotin, Heavy |
| | | | | | metal |
| Triphenyltin fluoride | 379-52-2 | 83602 | 2212 | Antifoulant | Organotin, Heavy |
| | | | | | metal |
| Tripropyltin methacrylate | 4154-35-2 | 83202 | NA 830 | N/A | Organotin, Heavy |
| | | | | | metal |
| Zinc arsenate | 13464-44-3 | 13301 | NA 125 | Herbicide, | Inorganic-Arsenic, |
| | | | | Insecticide, | Heavy metal, |
| | | | | Rodenticide | Inorganic-Zinc |
| Zinc arsenite | 28837-97-0 | 13604 | NA 130 | Herbicide, | Inorganic-Arsenic, |
| | | | | Insecticide, | Inorganic-Zinc, |
| | | | | Rodenticide | Heavy metal |
| Zinc mercury chromate | 22323-45-1 | 21004 | NA 170 | Fungicide | Inorganic-Mercury, |
| | | | | | Inorganic- |
| | | | | | chromium(VI), |
| | | | | | Inorganic-Zinc, |
| | | | | | Heavy metal |
| Ammonium arsenate | 7784-44-3 | 13601 | 2380 | Herbicide, | Inorganic-arsenic, |
| | | | | Insecticide, | Heavy metal |
| | | | | Rodenticide | |
| Arsenic | 7440-38-2 | NA 121 | 710 | Herbicide, | Inorganic-arsenic, |
| | | | | Insecticide, | Heavy metal |
| | | | | Rodenticide | |
| Arsenic acid | 7778-39-4 | 6801 | 40 | Herbicide, | Inorganic-arsenic, |
| | | | | Insecticide, | Heavy metal |
| | | | | Rodenticide | |
| Arsenic disulfide | 56320-22-0 | 6901 | NA 98 | Herbicide, | Inorganic-arsenic, |
| | | | | Insecticide, | Heavy metal |
| | | | | Rodenticide | |
| Arsenic pentoxide | 1303-28-2 | 6802 | 631 | Fungicide, | Inorganic-arsenic, |
| | | | | Insecticide, | Heavy metal |
| | | | | Rodenticide, | |
| | | | | Herbicide | |
| Arsenic sulfide | 12612-21-4 | 6901 | NA 99 | Herbicide, | Inorganic-arsenic, |
| | | | | Insecticide, | Heavy metal |
| | | | | Rodenticide | |
| Arsenic trioxide | 1327-53-3 | 7001 | 42 | Herbicide, | Inorganic-arsenic, |
| | | | | Insecticide, | Heavy metal |
| | | | | Rodenticide | |
| Arsenic trisulfide | 1303-33-9 | 6901 | NA 97 | Herbicide, | Inorganic-arsenic, |
| | | | | Insecticide, | Heavy metal |
| | | | | Rodenticide | |
| Arsonic acid, ammonium salt | 58829-95-1 | ##### | NA 105 | Herbicide, | Inorganic-arsenic, |
| | | | | Insecticide, | Heavy metal |
| | | | | Wood | |
| | | | | Preservative | |
| Basic copper arsenate | 16102-92-4 | ##### | NA 132 | Herbicide, | Inorganic-arsenic, |
| | | | | Insecticide, | Heavy metal, |
| | | | | Rodenticide | Inorganic-Copper |
| Calcium arsenate | 7778-44-1 | 13501 | 96 | Herbicide, | Inorganic-arsenic, |
| | | | | Insecticide, | Heavy metal |
| | | | | Rodenticide, | |
| | | | | Molluscicide | |
| Calcium arsenite | 53404-59-4 | 13602 | NA 129 | Herbicide, | Inorganic-arsenic, |
| | | | | Insecticide, | Heavy metal |
| | | | | Rodenticide | |
| Copper arsenate | 10103-61-4 | 22801 | NA 178 | Herbicide, | Inorganic-arsenic, |
| | | | | Insecticide, | Heavy metal, |
| | | | | Rodenticide | Inorganic-Copper |
| Copper arsenite | 10290-12-7 | 22401 | NA 177 | Herbicide, | Inorganic-arsenic, |
| | | | | Insecticide, | Heavy metal, |
| | | | | Rodenticide | Inorganic-Copper |
| Cupric acetoarsenite | 12002-03-8 | 22601 | 2485 | Wood | Inorganic-arsenic, |
| | | | | Preservative | Heavy metal, |
| | | | | | Inorganic-Copper |
| Lead arsenate | 7784-40-9 | 13503 | 353 | Herbicide, | Inorganic-arsenic, |
| | | | | Insecticide, | Inorganic-lead, |
| | | | | Rodenticide | Heavy metal |
| Lead arsenate, basic | 1327-31-7 | 13502 | 354 | Herbicide, | Inorganic-arsenic, |
| | | | | Insecticide, | Inorganic-lead, |
| | | | | Rodenticide | Heavy metal |
| Magnesium arsenate | 10103-50-1 | 13504 | NA 126 | Herbicide, | Inorganic-arsenic, |
| | | | | Insecticide, | Heavy metal |
| | | | | Rodenticide | |
| Manganese arsenate | 7784-38-5 | 13506 | NA 127 | Herbicide, | Inorganic-arsenic, |
| | | | | Insecticide, | Heavy metal |
| | | | | Rodenticide | |
| Paris green | 12002-03-8 | 22601 | 460 | Wood | Inorganic-Arsenic, |
| | | | | Preservative | Inorganic-Copper, |
| | | | | | Heavy metal |
| Potassium arsenite | 10124-50-2 | 13605 | NA 131 | Herbicide, | Inorganic-arsenic, |
| | | | | Insecticide, | Heavy metal |
| | | | | Rodenticide | |
| Potassium arsenite | 13464-35-2 | 13605 | NA 132 | Herbicide, | Inorganic-arsenic, |
| | | | | Insecticide, | Heavy metal |
| | | | | Rodenticide | |
| Potassium hexafluoroarsenate | 17029-22-0 | ##### | NA 160 | N/A | Inorganic-arsenic, |
| | | | | | Heavy metal |
| Sodium arsenate | 13464-38-5 | 13505 | 283 | Herbicide, | Inorganic-arsenic, |
| | | | | Insecticide, | Heavy metal |
| | | | | Rodenticide | |
| Sodium arsenite | 7784-46-5 | 13603 | 534 | Herbicide, | Inorganic-arsenic, |
| | | | | Insecticide, | Heavy metal |
| | | | | Rodenticide, | |
| | | | | Fungicide | |
| Sodium pyroarsenate | 13464-42-1 | 13401 | 2864 | Wood | Inorganic-arsenic, |
| | | | | Preservative, | Heavy metal |
| | | | | Fungicide | |
| Sodium thioarsenate | 17367-56-5 | 13507 | NA 128 | N/A | Inorganic-arsenic, |
| (Na3AsO3S) | | | | | Heavy metal |
| Zinc arsenate | 13464-44-3 | 13301 | NA 125 | Herbicide, | Inorganic-Arsenic, |
| | | | | Insecticide, | Heavy metal, |
| | | | | Rodenticide | Inorganic-Zinc |
| Zinc arsenite | 28837-97-0 | 13604 | NA 130 | Herbicide, | Inorganic-Arsenic, |
| | | | | Insecticide, | Inorganic-Zinc, |
| | | | | Rodenticide | Heavy metal |
| Antimony | 7440-36-0 | NA 516 | 2388 | N/A | Antimony |
| Antimony potassium tartrate | 28300-74-5 | 6201 | 3057 | Insecticide | Antimony |
| Diphenylstibene 2- | 5035-58-5 | 6202 | NA 82 | Microbiocide | Antimony |
| ethylhexanoate | | | | | |
| 10,10′-Oxybisphenoxyarsine | 58-36-6 | 12601 | 1402 | Fungicide, | Organoarsenic, |
| | | | | Microbiocide | Heavy metal |
| Arsenosobenzene | 637-03-6 | 7101 | NA 100 | N/A | Organoarsenic, |
| | | | | | Heavy metal |
| Arsonic acid, (4-aminophenyl)- | 98-50-0 | ##### | NA 109 | N/A | Organoarsenic, |
| | | | | | Heavy metal |
| Cacodylic acid | 75-60-5 | 12501 | 32 | Herbicide, | Organoarsenic, |
| | | | | Defoliant | Heavy metal |
| Calcium acid methanearsonate | 5902-95-4 | 13806 | 101 | Herbicide, | Organoarsenic, |
| | | | | Defoliant | Heavy metal |
| Calcium methanearsonate | 6423-72-9 | 13809 | NA 136 | Herbicide, | Organoarsenic, |
| | | | | Defoliant | Heavy metal |
| Calcium propanearsonate | 126-94-3 | 13801 | NA 133 | Herbicide, | Organoarsenic, |
| | | | | Defoliant | Heavy metal |
| Dodecyl ammonium | 53404-47-0 | 13805 | 857 | Herbicide, | Organoarsenic, |
| methanearsonate | | | | Defoliant | Heavy metal |
| DSMA | 144-21-8 | 13802 | 251 | Herbicide, | Organoarsenic, |
| | | | | Defoliant | Heavy metal |
| Methane arsonic acid (MAA) | 124-58-3 | ##### | NA 27 | N/A | Organoarsenic, |
| | | | | | Heavy metal |
| Monoammonium | 2321-53-1 | 13808 | NA 135 | Herbicide, | Organoarsenic, |
| methanearsonate | | | | Defoliant | Heavy metal |
| MSMA | 2163-80-6 | 13803 | 34 | Herbicide, | Organoarsenic, |
| | | | | Defoliant | Heavy metal |
| Octylammonium | 6379-37-9 | 13804 | 27 | Herbicide, | Organoarsenic, |
| methanearsonate | | | | Defoliant | Heavy metal |
| Phenarsazine chloride | 578-94-9 | 63901 | 1342 | N/A | Organoarsenic, |
| | | | | | Heavy metal |
| Phenarsazine oxide | 4095-45-8 | 12602 | NA 122 | N/A | Organoarsenic, |
| | | | | | Heavy metal |
| Sodium cacodylate | 124-65-2 | 12502 | 1673 | Herbicide, | Organoarsenic, |
| | | | | Defoliant, | Heavy metal |
| | | | | Rodenticide | |
| Triethanolamine cacodylate | 69484-14-6 | 12503 | NA 121 | Herbicide, | Organoarsenic, |
| | | | | Defoliant | Heavy metal |
| Triethanolamine | 5902-97-6 | 13807 | NA 134 | Herbicide, | Organoarsenic, |
| methanearsonate | | | | Defoliant | Heavy metal |
| Calomel | 10112-91-1 | 52201 | 100 | Fungicide | Inorganic-Mercury, |
| | | | | | Heavy metal |
| Hydroxymercury cresol | 12379-66-7 | 46001 | NA 390 | Microbiocide | Inorganic-Mercury, |
| | | | | | Heavy metal, Phenols |
| Mercuric acetate | 1600-27-7 | 52104 | NA 441 | N/A | Inorganic-Mercury, |
| | | | | | Heavy metal |
| Mercuric chloride | 7487-94-7 | 52001 | 372 | Fungicide | Inorganic-Mercury, |
| | | | | | Heavy metal |
| Mercuric dimethyl | 15415-64-2 | 34808 | 709 | Microbiocide | Inorganic-Mercury, |
| dithiocarbamate | | | | | Heavy metal, |
| | | | | | Dithiocarbamate |
| Mercuric iodide | 7774-29-0 | 52003 | NA 438 | N/A | Inorganic-Mercury, |
| | | | | | Heavy metal |
| Mercuric oleate | 1191-80-6 | 52105 | 3276 | N/A | Inorganic-Mercury, |
| | | | | | Heavy metal |
| Mercuric oxide | 21908-53-2 | 52102 | 955 | Fungicide | Inorganic-Mercury, |
| | | | | | Heavy metal |
| Mercury | 7439-97-6 | 52301 | NA 26 | N/A | Inorganic-Mercury, |
| | | | | | Heavy metal |
| Mercury naphthenate | 1336-96-5 | 52101 | NA 439 | Wood | Inorganic-Mercury, |
| | | | | Preservative, | Heavy metal |
| | | | | Insecticide, | |
| | | | | Fungicide | |
| Mercury pentanedione | 14024-55-6 | 52103 | NA 440 | N/A | Inorganic-Mercury, |
| | | | | | Heavy metal |
| Mercury phenate | 588-66-9 | 52106 | NA 442 | N/A | Inorganic-Mercury, |
| | | | | | Heavy metal |
| p-tert- | 53433-01-5 | 52002 | NA 437 | Microbiocide | Inorganic-Mercury, |
| Octylphenoxyethoxyethyl | | | | | Heavy metal |
| dimethyl benzyl ammonium | | | | | |
| mercuric chloride | | | | | |
| Potassium mercuric iodide | 7783-33-7 | 52107 | NA 443 | N/A | Inorganic-Mercury, |
| | | | | | Heavy metal |
| Zinc mercury chromate | 22323-45-1 | 21004 | NA 170 | Fungicide | Inorganic-Mercury, |
| | | | | | Inorganic- |
| | | | | | chromium(VI), |
| | | | | | Inorganic-Zinc, |
| | | | | | Heavy metal |
| (3-Ethoxypropyl)mercury | 6012-84-6 | ##### | NA 147 | Fungicide | Organomercury, |
| bromide | | | | | Heavy metal |
| (3-Hydroxy-2- | 69653-69-6 | ##### | NA 153 | N/A | Organomercury, |
| methoxypropyl)mercuric | | | | | Heavy metal |
| acetate | | | | | |
| 2-(acetoxymercuri)ethanol | 4665-55-8 | 41501 | NA 356 | N/A | Organomercury, |
| | | | | | Heavy metal |
| 3-(Hydroxymercuri)-4-nitro-o- | 53404-55-0 | 52604 | NA 447 | Microbiocide | Organomercury, |
| phenol, sodium salt | | | | | Heavy metal |
| Chloromethoxy propyl | 1319-86-4 | 18401 | 887 | N/A | Organomercury, |
| mercuric acetamide | | | | | Heavy metal |
| Di(phenylmercuri) ammonium | 18467-88-4 | 66002 | NA 551 | N/A | Organomercury, |
| propionate | | | | | Heavy metal |
| Di(phenylmercuric) dodecenyl | 27236-65-3 | 66001 | 501 | Microbiocide, | Organomercury, |
| succinate | | | | Fungicide | Heavy metal |
| Ethylmercuric phosphate | 2235-25-8 | 41505 | 357 | N/A | Organomercury, |
| | | | | | Heavy metal |
| Ethylmercurichlorendiimide | 2597-93-5 | 45301 | NA 383 | N/A | Organomercury, |
| | | | | | Heavy metal |
| Ethylmercurithiosalicylic acid | 148-61-8 | 78902 | NA 724 | Fungicide | Organomercury, |
| | | | | | Heavy metal |
| Ethylmercury 2,3- | 2597-92-4 | 41507 | NA 361 | Fungicide | Organomercury, |
| dihydroxypropylmercaptide | | | | | Heavy metal |
| Ethylmercury acetate | 109-62-6 | 41502 | NA 357 | Fungicide | Organomercury, |
| | | | | | Heavy metal |
| Ethylmercury bromide | 107-26-6 | ##### | NA 149 | Fungicide | Organomercury, |
| | | | | | Heavy metal |
| Ethylmercury chloride | 107-27-7 | 41503 | NA 358 | Fungicide | Organomercury, |
| | | | | | Heavy metal |
| Ethylmercury | 22232-28-6 | 41504 | NA 359 | Microbiocide | Organomercury, |
| pentachlorophenate | | | | | Chlorinated Phenol, |
| | | | | | Heavy metal |
| Hydroxymercuri-o-nitrophenol | 30284-78-7 | 52601 | 2606 | Microbiocide | Organomercury, |
| | | | | | Heavy metal |
| Hydroxymercuri-o-nitrophenol, | 1300-34-1 | 52602 | NA 445 | Microbiocide | Organomercury, |
| sodium salt | | | | | Heavy metal |
| Hydroxymercurichlorophenols | 53466-93-6 | 46002 | NA 391 | Microbiocide | Organomercury, |
| | | | | | Heavy metal |
| Hydroxymercuriphenyl | 53466-95-8 | 46003 | NA 392 | Microbiocide | Organomercury, |
| ammonium triethanolamine | | | | | Heavy metal |
| Lactoxymercuriphenyl | 53466-98-1 | 51901 | NA 429 | Microbiocide | Organomercury, |
| ammonium lactate | | | | | Heavy metal |
| Methoxyethyl mercury chloride | 123-88-6 | NA 212 | NA 211 | Fungicide | Organomercury |
| Methoxyethyl mercury silicate | 64491-92-5 | NA 208 | NA 208 | Fungicide | Organomercury |
| Methoxyethylmercury acetate | 151-38-2 | 41508 | NA 362 | Fungicide | Organomercury, |
| | | | | | Heavy metal |
| Methylmercurichlorendimide | 5902-79-4 | 45302 | NA 384 | N/A | Organomercury, |
| | | | | | Heavy metal |
| Methylmercury 2,3- | 2597-95-7 | 51905 | NA 433 | Fungicide | Organomercury, |
| dihydroxypropylmercaptide | | | | | Heavy metal |
| Methylmercury 8- | 86-85-1 | 51902 | NA 430 | Fungicide | Organomercury, |
| hydroxyquinolinate | | | | | Heavy metal |
| Methylmercury acetate | 108-07-6 | 51903 | NA 431 | Fungicide | Organomercury, |
| | | | | | Heavy metal |
| Methylmercury benzoate | 3626-13-9 | 51904 | NA 432 | Fungicide | Organomercury, |
| | | | | | Heavy metal |
| Methylmercury dicyano | 502-39-6 | 51909 | 454 | Fungicide | Organomercury, |
| diamide | | | | | Heavy metal |
| Methylmercury hydroxide | 1184-57-2 | 51906 | NA 434 | Fungicide | Organomercury, |
| | | | | | Heavy metal |
| Methylmercury nitrile | 2597-97-9 | 51907 | NA 435 | Fungicide | Organomercury, |
| | | | | | Heavy metal |
| Methylmercury propionate | 1460883 | 51908 | NA 436 | Fungicide | Organomercury, |
| | | | | | Heavy metal |
| N-(Ethylmercury)-p- | 517-16-8 | 41506 | NA 360 | Fungicide | Organomercury, |
| toluenesulfonanilide | | | | | Heavy metal |
| N-(Phenylmercuri) urea | 2279-64-3 | ##### | NA 160 | Herbicide | Organomercury, |
| | | | | | Heavy metal |
| N- | 5980-82-5 | 66009 | NA 556 | Fungicide | Organomercury, |
| (Phenylmercuri)ethylenediamine | | | | | Heavy metal |
| o-(Chloromercuri)phenol | 90-03-9 | ##### | NA 135 | Microbiocide | Organomercury, |
| | | | | | Heavy metal |
| o-(Hydroxymercuri)benzoic | 5722-59-8 | 52603 | NA 446 | Microbiocide | Organomercury, |
| acid, cyclic anhydride | | | | | Heavy metal |
| Phenylmercuriammonium | 53404-68-5 | 66023 | NA 567 | Fungicide | Organomercury, |
| propionate | | | | | Heavy metal |
| Phenylmercuric 2- | 13302-00-6 | 66024 | NA 568 | Fungicide, | Organomercury, |
| ethylhexanoate | | | | Microbiocide | Heavy metal |
| Phenylmercuric borate | 6273-99-0 | 66005 | NA 552 | Fungicide, | Organomercury, |
| | | | | Microbiocide | Heavy metal |
| Phenylmercuric carbonate | 53404-69-6 | 66006 | NA 553 | Fungicide, | Organomercury, |
| | | | | Microbiocide | Heavy metal |
| Phenylmercuric chloride | 100-56-1 | 66007 | NA 554 | Fungicide, | Organomercury, |
| | | | | Microbiocide | Heavy metal |
| Phenylmercuric | 32407-99-1 | 66008 | NA 555 | Microbiocide, | Organomercury, |
| dimethyldithiocarbamate | | | | Fungicide | Dithiocarbamate, |
| | | | | | Heavy metal |
| Phenylmercuric formamide | 22894-47-9 | 66010 | NA 557 | Fungicide, | Organomercury, |
| | | | | Microbiocide, | Heavy metal |
| | | | | Herbicide | |
| Phenylmercuric hydroxide | 100-57-2 | 66011 | NA 558 | Fungicide, | Organomercury, |
| | | | | Microbiocide, | Heavy metal |
| | | | | Herbicide | |
| Phenylmercuric lactate | 122-64-5 | 66012 | 1535 | Microbiocide, | Organomercury, |
| | | | | Fungicide, | Heavy metal |
| | | | | Herbicide | |
| Phenylmercuric | 5822-97-9 | 66013 | NA 559 | Fungicide, | Organomercury, |
| monoethanolammonium | | | | Microbiocide, | Heavy metal |
| acetate | | | | Herbicide | |
| Phenylmercuric | 53404-70-9 | 66014 | NA 560 | Fungicide, | Organomercury, |
| monoethanolammonium lactate | | | | Microbiocide, | Heavy metal |
| | | | | Herbicide | |
| Phenylmercuric naphthenate | 31632-68-5 | 66015 | NA 561 | Wood | Organomercury, |
| | | | | Preservative, | Heavy metal |
| | | | | Insecticide, | |
| | | | | Fungicide | |
| Phenylmercuric nitrate | 55-68-5 | 66016 | 477 | Microbiocide, | Organomercury, |
| | | | | Fungicide, | Heavy metal |
| | | | | Herbicide | |
| Phenylmercuric octanoate | 13864-38-5 | 66025 | NA 569 | Fungicide, | Organomercury, |
| | | | | Microbiocide, | Heavy metal |
| | | | | Herbicide | |
| Phenylmercuric oleate | 104-60-9 | 66022 | 853 | Microbiocide, | Organomercury, |
| | | | | Fungicide, | Heavy metal |
| | | | | Herbicide | |
| Phenylmercuric propionate | 103-27-5 | 66018 | NA 563 | Fungicide, | Organomercury, |
| | | | | Microbiocide, | Heavy metal |
| | | | | Herbicide | |
| Phenylmercuric salicylate | 28086-13-7 | 66019 | NA 564 | Fungicide, | Organomercury, |
| | | | | Microbiocide, | Heavy metal |
| | | | | Herbicide | |
| Phenylmercuric thiocyanate | 16751-55-6 | 66020 | NA 565 | Microbiocide, | Organomercury, |
| | | | | Fungicide, | Heavy metal |
| | | | | Herbicide | |
| Phenylmercuritriethanolammonium | 23319-66-6 | 66021 | NA 566 | Microbiocide, | Organomercury, |
| lactate | | | | Fungicide, | Heavy metal |
| | | | | Herbicide | |
| Phenylmercury 8- | 14354-56-4 | 66017 | NA 562 | Fungicide, | Organomercury, |
| hydroxyquinolinolate | | | | Microbiocide, | Heavy metal |
| | | | | Herbicide | |
| Phenylmercury benzoate | 94-43-9 | 51900 | NA 428 | Fungicide, | Organomercury, |
| | | | | Microbiocide, | Heavy metal |
| | | | | Herbicide | |
| Phenylmercury nitrate | 2227736 | NA 209 | NA 208 | Fungicide | Organomercury |
| PMA | 62-38-4 | 66003 | 491 | Fungicide, | Organomercury, |
| | | | | Microbiocide, | Heavy metal |
| | | | | Herbicide | |
| PMA, other related | NA 1106 | NA 164 | 90491 | Fungicide, | Organomercury, |
| | | | | Herbicide, | Heavy metal |
| | | | | Microbiocide | |
| PMAA | 53404-67-4 | 66004 | 492 | Fungicide, | Organomercury, |
| | | | | Microbiocide | Heavy metal |
| Pyridylmercuric acetate | 1334-77-6 | 69501 | NA 661 | N/A | Organomercury, |
| | | | | | Heavy metal |
| Pyridylmercuric chloride | 1334-75-4 | 69502 | NA 662 | N/A | Organomercury, |
| | | | | | Heavy metal |
| Sodium | 54-64-8 | 78901 | NA 723 | N/A | Organomercury, |
| ethylmercurithiosalicylate | | | | | Heavy metal |
| Tolylmercuric acetate | 1300-78-3 | ##### | NA 150 | Fungicide | Organomercury, |
| | | | | | Heavy metal |
| Anilinocadmium dilactate | 19651-91-3 | 64601 | NA 548 | N/A | Inorganic-Cadmium, |
| | | | | | Heavy metal |
| Cadmium | 7440-43-9 | NA 460 | 2438 | N/A | Inorganic-Cadmium, |
| | | | | | Heavy metal |
| Cadmium carbonate | 513-78-0 | 12901 | 93 | N/A | Inorganic-Cadmium, |
| | | | | | Heavy metal |
| Cadmium chloride | 10108-64-2 | 12902 | 94 | N/A | Inorganic-Cadmium, |
| | | | | | Heavy metal |
| Cadmium cocoate | 72869-63-7 | NA 459 | 3085 | N/A | Inorganic-Cadmium, |
| | | | | | Heavy metal |
| Cadmium oxide | 1306-19-0 | ##### | NA 130 | N/A | Inorganic-Cadmium, |
| | | | | | Heavy metal |
| Cadmium perborate | NA 969 | NA 461 | 3086 | N/A | Inorganic-Cadmium, |
| | | | | | Heavy metal |
| Cadmium sebacate | 939499 | 12903 | 699 | N/A | Inorganic-Cadmium, |
| | | | | | Heavy metal |
| Cadmium succinate | 141-00-4 | 12904 | 92 | N/A | Inorganic-Cadmium, |
| | | | | | Heavy metal |
| Cadmium sulfate | 10124-36-4 | 12905 | NA 123 | N/A | Inorganic-Cadmium, |
| | | | | | Heavy metal |
| Cadmium yellow pigments | NA 967 | NA 448 | 2439 | N/A | Inorganic-Cadmium, |
| | | | | | Heavy metal |
| Crag turf fungicide 531 | 12001-20-6 | 21006 | NA 171 | Fungicide | Inorganic-Cadmium, |
| (Cd—Cu—Ca—Zn-Chromate) | | | | | Inorganic- |
| | | | | | Chromium(VI), |
| | | | | | Inorganic-Zinc, |
| | | | | | Heavy metal |
| Lauric acid, barium cadmium | 15337-60-7 | NA 118 | 3676 | Adjuvant | Inorganic-Cadmium, |
| salt | | | | | Heavy metal |
| Ammonium dichromate | 2149701 | 68305 | NA 607 | Wood | Inorganic- |
| | | | | Preservative | Chromium(VI), |
| | | | | | Heavy metal |
| Chromated zinc chloride | NA 1026 | 87801 | 1187 | Insecticide, | Inorganic- |
| | | | | Herbicide, | Chromium(VI), |
| | | | | Microbiocide | Inorganic-Zinc, |
| | | | | | Heavy metal |
| Chromic acetate | 1066-30-4 | 21102 | NA 172 | Wood | Inorganic- |
| | | | | Preservative | Chromium(VI), |
| | | | | | Heavy metal |
| Chromic acid | 7738-94-5 | 21101 | 1188 | Wood | Inorganic- |
| | | | | Preservative | Chromium(VI), |
| | | | | | Heavy metal |
| Chromic oxide | 1308-38-9 | 21103 | NA 173 | Wood | Inorganic- |
| | | | | Preservative | Chromium(VI), |
| | | | | | Heavy metal |
| Copper-zinc chromate complex | 1344-74-7 | 21003 | 163 | Fungicide, | Inorganic- |
| | | | | Wood | Chromium(VI), |
| | | | | Preservative | Inorganic-Zinc, |
| | | | | | Heavy metal, |
| | | | | | Inorganic-Copper |
| Crag turf fungicide 531 | 12001-20-6 | 21006 | NA 171 | Fungicide | Inorganic-Cadmium, |
| (Cd—Cu—Ca—Zn-Chromate) | | | | | Inorganic- |
| | | | | | Chromium(VI), |
| | | | | | Inorganic-Zinc, |
| | | | | | Heavy metal |
| Potassium chromate | 7789-00-6 | 68301 | 700 | Wood | Inorganic- |
| | | | | Preservative | Chromium(VI), |
| | | | | | Heavy metal |
| Potassium dichromate | 7778-50-9 | 68302 | 2167 | Wood | Inorganic- |
| | | | | Preservative | Chromium(VI) |
| Sodium bichromate dihydrate | 7789-12-0 | 68306 | 3980 | Wood | Inorganic- |
| | | | | Preservative | Chromium(VI), |
| | | | | | Inorganic |
| Sodium chromate | 2144646 | 68303 | 1241 | Wood | Inorganic- |
| | | | | Preservative | Chromium(VI), |
| | | | | | Heavy metal |
| Sodium dichromate | 10588-01-9 | 68304 | 3395 | Wood | Inorganic- |
| | | | | Preservative | Chromium(VI), |
| | | | | | Heavy metal |
| Zinc mercury chromate | 22323-45-1 | 21004 | NA 170 | Fungicide | Inorganic-Mercury, |
| | | | | | Inorganic- |
| | | | | | chromium(VI), |
| | | | | | Inorganic-Zinc, |
| | | | | | Heavy metal |
| Acetic acid, copper (2+) salt | 142-71-2 | NA 697 | 3013 | N/A | Inorganic-Copper |
| Basic copper arsenate | 16102-92-4 | ##### | NA 132 | Herbicide, | Inorganic-arsenic, |
| | | | | Insecticide, | Heavy metal, |
| | | | | Rodenticide | Inorganic-Copper |
| Bordeaux mixture | NA 240 | NA 478 | 80 | Fungicide | Inorganic-Copper |
| Chlorophyllin | 11006-34-1 | NA 191 | NA 190 | Fungicide, | Inorganic-Copper, |
| | | | | Microbiocide | Botanical |
| Copper | 7440-50-8 | 22501 | 714 | Fungicide | Inorganic-Copper |
| Copper (from triethanolamine | 82027-59-6 | 24403 | NA 198 | Algaecide | Inorganic-Copper |
| complex) | | | | | |
| Copper 2-ethylhexanoate | 22221-10-9 | 41201 | 2235 | Fungicide | Inorganic-Copper |
| Copper 3-phenylsalicylate | 1250682 | 23801 | NA 191 | N/A | Inorganic-Copper |
| Copper 8-quinolinoleate | 10380-28-6 | 24002 | 159 | Fungicide, | Inorganic-Copper |
| | | | | Microbiocide | |
| Copper abietate | 10248-55-2 | 23301 | NA 184 | Fungicide | Inorganic-Copper |
| Copper acetate | 831632 | 44002 | 147 | Fungicide | Inorganic-Copper |
| Copper ammonium acetate | NA 1544 | NA 225 | NA 225 | Fungicide | Inorganic-Copper |
| Copper ammonium carbonate | 33113-08-5 | 22703 | 1762 | Fungicide | Inorganic-Copper |
| Copper ammonium complex | 16828-95-8 | 22702 | 3550 | Fungicide | Inorganic-Copper |
| Copper arsenate | 10103-61-4 | 22801 | NA 178 | Herbicide, | Inorganic-arsenic, |
| | | | | Insecticide, | Heavy metal, |
| | | | | Rodenticide | Inorganic-Copper |
| Copper arsenite | 10290-12-7 | 22401 | NA 177 | Herbicide, | Inorganic-arsenic, |
| | | | | Insecticide, | Heavy metal, |
| | | | | Rodenticide | Inorganic-Copper |
| Copper as elemental from | 50376-91-5 | 24404 | NA 199 | N/A | Inorganic-Copper |
| copper - etidronic acid complex | | | | | |
| Copper beta cyclodextrin | NA 1366 | NA 193 | NA 193 | Fungicide | Inorganic-Copper |
| hydroxide | | | | | |
| Copper bronze powder | 7440-50-8 | 22501 | 1457 | Antifoulant | Inorganic-Copper |
| Copper carbonate, basic | 1184-64-1 | 22901 | 60 | Fungicide, | Inorganic-Copper |
| | | | | Algaecide, | |
| | | | | Insecticide | |
| Copper chloride (anhydrous) | 1344-67-8 | 23802 | NA 192 | N/A | Inorganic-Copper |
| Copper chloride (dihydrate) | 10125-13-0 | 23701 | NA 190 | N/A | Inorganic-Copper |
| Copper citrate | 10402-15-0 | 44005 | 1406 | N/A | Inorganic-Copper |
| Copper citrate chelate | NA 961 | NA 387 | 3547 | N/A | Inorganic-Copper |
| Copper complex with ammonia | 67989-88-2 | ##### | NA 130 | Fungicide | Inorganic-Copper |
| and ethylene diamine | | | | | |
| tetraacetate | | | | | |
| Copper cresylate | 12379-42-9 | 23001 | NA 181 | Fungicide | Inorganic-Copper |
| Copper | 53404-24-3 | 41202 | NA 349 | N/A | Inorganic-Copper |
| dehydroabietylammonium 2- | | | | | |
| ethylhexanoate | | | | | |
| Copper dihydrazinium sulfate | 33271-65-7 | 50504 | 753 | N/A | Inorganic-Copper |
| Copper ethanolamine complex | 14215-52-2 | 24409 | NA 201 | Algaecide | Inorganic-Copper |
| Copper ethanolamine | NA 1044 | NA 146 | 3551 | Algaecide | Inorganic-Copper |
| complexes, mixed | | | | | |
| Copper ethylenediamine | 13426-91-0 | 24407 | 3549 | Herbicide | Inorganic-Copper |
| complex | | | | | |
| Copper gluconate chelate | 814-91-5 | 23305 | 3548 | N/A | Inorganic-Copper |
| Copper hydrazinium sulfate | 53433-02-6 | 50501 | NA 418 | Fungicide | Inorganic-Copper |
| Copper hydroxide | 20427-59-2 | 23401 | 151 | Fungicide, | Inorganic-Copper |
| | | | | Microbiocide, | |
| | | | | Nematicide | |
| Copper hydroxide - | NA 1017 | NA 105 | 1826 | N/A | Inorganic-Copper |
| triethanolamine complex | | | | | |
| Copper isodecanoate | 84082-88-2 | 41221 | NA 354 | N/A | Inorganic-Copper |
| Copper isononanoate | 72915-82-3 | 41211 | NA 352 | N/A | Inorganic-Copper |
| Copper isooctanoate | 88859-94-3 | 41204 | NA 350 | N/A | Inorganic-Copper |
| Copper lactate | 16039-52-4 | 23302 | NA 185 | N/A | Inorganic-Copper |
| Copper linoleate | 7721-15-5 | 23303 | 1110 | N/A | Inorganic-Copper |
| Copper metaborate | 39290-85-2 | 22802 | NA 179 | N/A | Inorganic-Copper |
| Copper monoethanolamine | NA 960 | NA 384 | 3552 | N/A | Inorganic-Copper |
| complex | | | | | |
| Copper naphthenate | 1338-02-9 | 23102 | 153 | Wood | Inorganic-Copper |
| | | | | Preservative, | |
| | | | | Insecticide, | |
| | | | | Fungicide, Dog | |
| | | | | and Cat | |
| | | | | Repellent | |
| Copper naphthenate | 1338-02-9 | 6000 | NA 172 | Wood | Inorganic-Copper |
| | | | | Preservative, | |
| | | | | Insecticide, | |
| | | | | Fungicide, Dog | |
| | | | | and Cat | |
| | | | | Repellent | |
| Copper naphthenate | 1338-02-9 | 6300 | NA 172 | Wood | Inorganic-Copper |
| | | | | Preservative, | |
| | | | | Insecticide, | |
| | | | | Fungicide, Dog | |
| | | | | and Cat | |
| | | | | Repellent | |
| Copper nitroacetate | 22221-12-1 | 23201 | NA 183 | N/A | Inorganic-Copper |
| Copper octanoate | 20543-04-8 | 23306 | 5225 | Fungicide | Inorganic-Copper |
| Copper oleate | 10402-16-1 | 23304 | 154 | Fungicide | Inorganic-Copper |
| Copper oxide (ic) | 1317-38-0 | 42401 | 2231 | Fungicide, | Inorganic-Copper |
| | | | | Insecticide | |
| Copper oxide (ous) | 1317-39-1 | 25601 | 175 | Fungicide, | Inorganic-Copper |
| | | | | Insecticide | |
| Copper oxychloride | 1332-40-7 | 8001 | 156 | Fungicide | Inorganic-Copper |
| Copper oxychloride | 1332-65-6 | 23501 | NA 186 | Fungicide | Inorganic-Copper |
| (Cu2Cl(OH)3) | | | | | |
| Copper oxychloride sulfate | 8012-69-9 | 23503 | 158 | Fungicide | Inorganic-Copper |
| Copper oxysulfate | 12158-97-3 | 23504 | NA 188 | N/A | Inorganic-Copper |
| Copper oxysulfate | 82010-79-5 | 23504 | NA 189 | N/A | Inorganic-Copper |
| Copper pentachlorophenate | 15773-35-0 | 63011 | NA 512 | Wood | Chlorinated Phenol, |
| | | | | Preservative, | Inorganic-Copper |
| | | | | Microbiocide, | |
| | | | | Algaecide, | |
| | | | | Fungicide | |
| Copper phosphate | 10103-48-7 | 22902 | NA 180 | N/A | Inorganic-Copper |
| Copper phthalocyanine | 147-14-8 | NA 381 | 2479 | Dye | Inorganic-Copper |
| Copper pyrophosphate | 10102-90-6 | 69701 | NA 663 | N/A | Inorganic-Copper |
| Copper salts of fatty and rosin | 9007-39-0 | 23104 | 155 | Fungicide | Inorganic-Copper |
| acids | | | | | |
| Copper salts of the acids of tall | 61789-22-8 | 23103 | NA 182 | N/A | Inorganic-Copper |
| oil | | | | | |
| Copper silicate | 1344-72-5 | 24301 | NA 197 | N/A | Inorganic-Copper |
| Copper sodium sulfate- | NA 1023 | NA 121 | 755 | N/A | Inorganic-Copper |
| phosphate complex | | | | | |
| Copper sulfate (anhydrous) | 7758-98-7 | 24408 | 1778 | Fungicide, | Inorganic-Copper |
| | | | | Algaecide, | |
| | | | | Molluscicide | |
| Copper sulfate (basic) | 1344-73-6 | 8101 | 162 | Fungicide, | Inorganic-Copper |
| | | | | Algaecide, | |
| | | | | Molluscicide | |
| Copper sulfate (pentahydrate) | 7758-99-8 | 24401 | 161 | Algaecide, | Inorganic-Copper |
| | | | | Fungicide, | |
| | | | | Insecticide, | |
| | | | | Water | |
| | | | | Treatment, | |
| | | | | Molluscicide, | |
| | | | | Nematicide | |
| Copper sulfate ethylene | NA 1036 | NA 132 | 2480 | N/A | Inorganic-Copper |
| diamine | | | | | |
| Copper sulfate, monohydrate | 10257-54-2 | 24402 | 1789 | Fungicide, | Inorganic-Copper |
| | | | | Algaecide, | |
| | | | | Molluscicide | |
| Copper sulfate, tri-basic | NA 1384 | NA 195 | NA 194 | Herbicide, | Inorganic-Copper |
| | | | | Algaecide, | |
| | | | | Fungicide, | |
| | | | | Water | |
| | | | | Treatment | |
| Copper triethanolamine | 68027-59-6 | NA 125 | 1615 | Algaecide | Inorganic-Copper |
| complex | | | | | |
| Copper-zinc chromate complex | 1344-74-7 | 21003 | 163 | Fungicide, | Inorganic- |
| | | | | Wood | Chromium(VI), |
| | | | | Preservative | Inorganic-Zinc, |
| | | | | | Heavy metal, |
| | | | | | Inorganic-Copper |
| Copper-zinc sulfate complex | 55072-57-6 | 8102 | 164 | Fungicide | Inorganic-Zinc, |
| | | | | | Inorganic-Copper |
| Copper-zinc sulfate complex, | NA 959 | NA 382 | 1751 | Fungicide | Inorganic-Zinc, |
| monohydrate | | | | | Inorganic-Copper |
| Cuprammonium | NA 1382 | NA 195 | NA 194 | Fungicide | Inorganic-Copper |
| Cupric acetoarsenite | 12002-03-8 | 22601 | 2485 | Wood | Inorganic-arsenic, |
| | | | | Preservative | Heavy metal, |
| | | | | | Inorganic-Copper |
| Cupric ferric subsulfate | 12168-20-6 | 42402 | NA 365 | Molluscicide | Inorganic-Copper |
| complex | | | | | |
| Cupric fluosilicate | 12062-24-7 | 75309 | NA 692 | Insecticide | Inorganic-Copper |
| Cupric gluconate | 527-09-3 | 24405 | 3117 | Fungicide | Inorganic-Copper |
| Cupric nitrate | 3251-23-8 | 76102 | 3118 | N/A | Inorganic-Copper |
| Cuprous and cupric oxide, | 82010-82-0 | 42403 | NA 366 | N/A | Inorganic-Copper |
| mixed | | | | | |
| Cuprous chloride (Cu2Cl2) | 7758-89-6 | ##### | 5597 | Fungicide | Inorganic-Copper |
| Cuprous iodide | 7681-65-4 | ##### | NA 902 | N/A | Inorganic-Copper |
| Cuprous sulfide | 22205-45-4 | ##### | NA 124 | N/A | Inorganic-Copper |
| Cuprous thiocyanate | 1111-67-7 | 25602 | 2108 | Microbiocide | Inorganic-Copper |
| Device (swimming pool | NA 1504 | NA 222 | NA 222 | Algaecide | Inorganic-Copper |
| algaecide, copper generating) | | | | | |
| no guarantee required | | | | | |
| Dihydrogen | 54453-03-1 | 24406 | NA 200 | N/A | Inorganic-Copper |
| {ethylenediaminetetraacetato(4-)} | | | | | |
| cuprate(2-) | | | | | |
| Disodium EDTA-copper | 14025-15-1 | NA 296 | 3173 | N/A | Inorganic-Copper |
| EDTA diammonium copper | 7379-26-2 | 39117 | 1613 | Algaecide | Inorganic-Copper |
| salt | | | | | |
| EDTA, copper complex | 12276-01-6 | 39105 | 1135 | Algaecide | Inorganic-Copper |
| Lignin sulfonic acid, copper | NA 933 | NA 126 | 1925 | N/A | Inorganic-Copper |
| salt | | | | | |
| Malachite (copper equivalent | 1319-53-5 | ##### | NA 155 | N/A | Inorganic-Copper |
| 57%) | | | | | |
| Mixed copper chelates or | NA 1437 | NA 213 | NA 212 | Fungicide | Inorganic-Copper |
| complex blend of copper salts | | | | | |
| N-(2-hydroxyethyl) ethylene | NA 1025 | NA 122 | 1130 | N/A | Inorganic-Copper |
| diamine triacetic acid, copper | | | | | |
| salt | | | | | |
| Neodecanoic acid, copper salt | 50315-14-5 | 97503 | NA 854 | N/A | Inorganic-Copper |
| Paris green | 12002-03-8 | 22601 | 460 | Wood | Inorganic-Arsenic, |
| | | | | Preservative | Inorganic-Copper, |
| | | | | | Heavy metal |
| Silver copper zeolite | 130328-19-7 | ##### | NA 110 | Microbiocide | Inorganic-Silver, |
| | | | | | Heavy metal, |
| | | | | | Inorganic-Copper |
| Tetracopper calcium | 1336-15-8 | 23502 | NA 187 | Fungicide | Inorganic-Copper |
| oxychloride | | | | | |
| Tricopper dichloride | 7076-63-3 | ##### | NA 152 | Microbiocide | Dithiocarbamate, |
| dimethyldithiocarbamate | | | | | Inorganic-Copper |
| Azocyclotin | 41083-11-8 | ##### | 2408 | Insecticide | Organotin, Heavy |
| | | | | | metal |
| Bis (tributyltin) adipate | 7437-35-6 | 83117 | 1990 | Antifoulant, | Organotin, Heavy |
| | | | | Microbiocide | metal |
| Bis (tributyltin) sulfide | 4808-30-4 | 83113 | 1886 | Antifoulant, | Organotin, Heavy |
| | | | | Microbiocide | metal |
| Bis (tributyltin) sulfone | NA 970 | NA 476 | 1665 | Antifoulant, | Organotin, Heavy |
| | | | | Microbiocide | metal |
| Bis(tributyltin) | 12379-54-3 | 83101 | NA 824 | Antifoulant, | Organotin, Heavy |
| dodecenylsuccinate | | | | Microbiocide | metal |
| Bis(tributyltin) salicylate | 22330-14-9 | 83102 | 5074 | Antifoulant, | Organotin, Heavy |
| | | | | Microbiocide | metal |
| Bis(tributyltin) succinate | 4644-96-6 | 83103 | 74 | Antifoulant, | Organotin, Heavy |
| | | | | Microbiocide | metal |
| Bis(tributyltin) sulfosalicylate | 4419-22-1 | 83104 | NA 825 | Antifoulant, | Organotin, Heavy |
| | | | | Microbiocide | metal |
| Bis(tripropyltin) oxide | 1067-29-4 | 83201 | NA 829 | Microbiocide, | Organotin, Heavy |
| | | | | Fungicide | metal |
| Cyhexatin | 13121-70-5 | ##### | 1638 | Insecticide | Organotin, Heavy |
| | | | | | metal |
| Decafentin | 15652-38-7 | ##### | NA 132 | N/A | Organotin, Heavy |
| | | | | | metal |
| Dibutyltin dichloride | 683-18-1 | 83123 | NA 828 | N/A | Organotin, Heavy |
| | | | | | metal |
| Ethylene oxide condensate of | 56573-85-4 | NA 126 | 1864 | Antifoulant, | Organotin, Heavy |
| abietylamine, tributyltin | | | | Microbiocide | metal |
| chloride | | | | | |
| Fenbutatin-oxide | 13356-08-6 | ##### | 1876 | Insecticide | Organotin, Heavy |
| | | | | | metal |
| Fentin hydroxide | 76-87-9 | 83601 | 599 | Fungicide, | Organotin, Heavy |
| | | | | Molluscicide, | metal |
| | | | | Herbicide | |
| Methylenebis(thiocyanate) with | 76397-81-4 | 81408 | NA 785 | Antifoulant, | Organotin, Heavy |
| TBTO | | | | Microbiocide | metal |
| Monotributylin salicylate | 4342-30-7 | 83122 | NA 827 | Antifoulant, | Organotin, Heavy |
| | | | | Microbiocide | metal |
| Poly (methylmethacrylate-co- | 26354-18-7 | 83119 | 5075 | Antifoulant, | Organotin, Heavy |
| tributyltin methacrylate) | | | | Microbiocide | metal |
| Tin Acrylate Polymers RC 626 | NA 1444 | NA 214 | NA 213 | Antifoulant | Organotin |
| & RC 627 | | | | | |
| Tri-n-butyltin maleate | 14275-57-1 | 83118 | 2185 | Antifoulant, | Organotin, Heavy |
| | | | | Microbiocide | metal |
| Tributyltin acetate | 56-36-0 | 83105 | 2097 | Antifoulant, | Organotin, Heavy |
| | | | | Microbiocide | metal |
| Tributyltin acrylate | 13331-52-7 | 83121 | NA 826 | Antifoulant, | Organotin, Heavy |
| | | | | Microbiocide | metal |
| Tributyltin benzoate | 4342-36-3 | 83106 | 1114 | Antifoulant, | Organotin, Heavy |
| | | | | Microbiocide | metal |
| Tributyltin chloride | 1461-22-9 | 83107 | 1891 | Antifoulant, | Organotin, Heavy |
| | | | | Microbiocide | metal |
| Tributyltin chloride complex of | 56573-85-4 | 83108 | 1412 | Antifoulant, | Organotin, Heavy |
| ethylene oxide condensate of | | | | Microbiocide | metal |
| abietylamine | | | | | |
| Tributyltin chloride, | NA 1012 | NA 970 | 938 | Antifoulant, | Organotin, Heavy |
| myristylamine salt | | | | Microbiocide | metal |
| Tributyltin fluoride | 29131 | 83112 | 1030 | Antifoulant, | Organotin, Heavy |
| | | | | Microbiocide, | metal |
| | | | | Fungicide | |
| Tributyltin isopropyl succinate | 53404-82-3 | 83115 | 1115 | Antifoulant, | Organotin, Heavy |
| | | | | Microbiocide | metal |
| Tributyltin linoleate | 24124-25-2 | 83109 | 1116 | Antifoulant, | Organotin, Heavy |
| | | | | Microbiocide | metal |
| Tributyltin methacrylate | 2155-70-6 | 83120 | 2179 | Antifoulant, | Organotin, Heavy |
| | | | | Microbiocide | metal |
| Tributyltin monopropylene | 53466-85-6 | 83110 | 1985 | Antifoulant, | Organotin, Heavy |
| glycol maleate | | | | Microbiocide | metal |
| Tributyltin naphthenate | 85409-17-2 | NA 211 | NA 210 | Fungicide | Organotin |
| Tributyltin neodecanoate | 28801-69-6 | 83111 | 1035 | Antifoulant, | Organotin, Heavy |
| | | | | Microbiocide | metal |
| Tributyltin oxide | 56-35-9 | 83001 | 569 | Antifoulant, | Organotin, Heavy |
| | | | | Microbiocide, | metal |
| | | | | Molluscicide, | |
| | | | | Fungicide | |
| Tributyltin resinate | 13387-91-2 | 83114 | 1732 | Antifoulant, | Organotin, Heavy |
| | | | | Microbiocide | metal |
| Tributyltin sulfide | 4808-30-4 | 83113 | 1352 | Antifoulant, | Organotin, Heavy |
| | | | | Microbiocide | metal |
| Trimethyltin chloride | 1066-45-1 | ##### | NA 161 | N/A | Organotin, Heavy |
| | | | | | metal |
| Triphenyltin acetate | 900-95-8 | ##### | NA 153 | Fungicide, | Organotin, Heavy |
| | | | | Herbicide | metal |
| Triphenyltin chloride | 639-58-7 | ##### | NA 153 | N/A | Organotin, Heavy |
| | | | | | metal |
| Triphenyltin fluoride | 379-52-2 | 83602 | 2212 | Antifoulant | Organotin, Heavy |
| | | | | | metal |
| Tripropyltin methacrylate | 4154-35-2 | 83202 | NA 830 | N/A | Organotin, Heavy |
| | | | | | metal |
| Basic lead silicate | 53466-66-3 | 48003 | NA 417 | N/A | Inorganic-Lead, |
| | | | | | Heavy metal |
| Lead | 7439-92-1 | NA 134 | 2638 | N/A | Inorganic-Lead, |
| | | | | | Heavy metal |
| Lead acetate | 301-04-2 | 48001 | NA 415 | N/A | Inorganic-Lead, |
| | | | | | Heavy metal |
| Lead arsenate | 7784-40-9 | 13503 | 353 | Herbicide, | Inorganic-arsenic, |
| | | | | Insecticide, | Inorganic-lead, |
| | | | | Rodenticide | Heavy metal |
| Lead arsenate, basic | 1327-31-7 | 13502 | 354 | Herbicide, | Inorganic-arsenic, |
| | | | | Insecticide, | Inorganic-lead, |
| | | | | Rodenticide | Heavy metal |
| Lead metasilicate | NA 938 | NA 138 | 1106 | N/A | Inorganic-Lead, |
| | | | | | Heavy metal |
| Lead monoxide | 10190-55-3 | NA 123 | 1271 | N/A | Inorganic-Lead, |
| | | | | | Heavy metal |
| Lead naphthenate | 61790-14-5 | 48004 | 3296 | N/A | Inorganic-Lead, |
| | | | | | Heavy metal |
| Litharge | 1317-36-8 | 48002 | NA 416 | N/A | Inorganic-Lead, |
| | | | | | Heavy metal |
| Alkyl*-1-(2-aminoethyl)-2- | NA 1188 | 46606 | NA 167 | Microbiocide | Inorganic-Nickel, |
| imidazoline acetate-nickel | | | | | Imidazoline |
| sulfate complex | | | | | |
| Maneb and nickel sulfate | 8005-46-7 | ##### | NA 158 | Fungicide | Dithiocarbamate, |
| hexahydrate (014505 + 050505) | | | | | Inorganic-Nickel |
| Nickel | 7440-02-0 | NA 121 | 762 | N/A | Inorganic-Nickel |
| Nickel diethyl hexyl acid | NA 928 | NA 53 | 3696 | N/A | Inorganic-Nickel |
| phosphate complex | | | | | |
| Nickel sulfate (anhydrous) | 7786-81-4 | 50508 | NA 420 | Fungicide | Inorganic-Nickel |
| Nickel sulfate hexahydrate | 10101-97-0 | 50505 | NA 419 | Fungicide | Inorganic-Nickel |
| Fatty acid*-silver complex | 24927-67-1 | 72505 | NA 675 | Microbiocide, | Inorganic-Silver, |
| *(100% C8) | | | | Fungicide, | Heavy metal |
| | | | | Herbicide | |
| Metallic silver | 7440-22-4 | 72501 | 2125 | Microbiocide, | Inorganic-Silver, |
| | | | | Fungicide, | Heavy metal |
| | | | | Herbicide | |
| Silver acetate | 563-63-3 | 72507 | NA 676 | Microbiocide, | Inorganic-Silver, |
| | | | | Fungicide, | Heavy metal |
| | | | | Herbicide | |
| Silver carbonate | 534-16-7 | 72509 | NA 678 | Microbiocide, | Inorganic-Silver, |
| | | | | Fungicide, | Heavy metal |
| | | | | Herbicide | |
| Silver chloride | 7783-90-6 | 72506 | 2324 | Microbiocide, | Inorganic-Silver, |
| | | | | Fungicide, | Heavy metal |
| | | | | Herbicide | |
| Silver copper zeolite | 130328-19-7 | ##### | NA 110 | Microbiocide | Inorganic-Silver, |
| | | | | | Heavy metal, |
| | | | | | Inorganic-Copper |
| Silver fluoride | 7775-41-9 | 72502 | NA 674 | Microbiocide, | Inorganic-Silver, |
| | | | | Fungicide, | Heavy metal |
| | | | | Herbicide | |
| Silver iodide, colloidal | 7783-96-2 | NA 124 | 1556 | Microbiocide, | Inorganic-Silver, |
| | | | | Fungicide, | Heavy metal |
| | | | | Herbicide | |
| Silver nitrate | 7761-88-8 | 72503 | 2856 | Microbiocide, | Inorganic-Silver, |
| | | | | Fungicide, | Heavy metal |
| | | | | Herbicide, | |
| | | | | Plant Growth | |
| | | | | Regulator | |
| Silver orthophosphate | 7784-09-0 | 72510 | NA 679 | Microbiocide, | Inorganic-Silver, |
| (Ag3PO4) | | | | Fungicide, | Heavy metal |
| | | | | Herbicide | |
| Silver oxide (Ag4O4) | 1301-96-8 | ##### | NA 111 | Microbiocide, | Inorganic-Silver, |
| | | | | Fungicide, | Heavy metal |
| | | | | Herbicide | |
| Silver oxide (Ag4O4) | 155645-89-9 | ##### | NA 111 | Microbiocide, | Inorganic-Silver, |
| | | | | Fungicide, | Heavy metal |
| | | | | Herbicide | |
| Silver sodium hydrogen | NA 1166 | 72560 | NA 164 | Microbiocide | Inorganic-Silver, |
| zirconium phosphate | | | | | Heavy metal |
| (Ag0.18Na0.57H0.25Zr2(PO4)3) | | | | | |
| Silver thiocyanate | 1701-93-5 | 68203 | NA 606 | Microbiocide, | Inorganic-Silver, |
| | | | | Fungicide, | Heavy metal |
| | | | | Herbicide | |
| Silver thiuronium acrylate co- | 53404-00-5 | 72701 | NA 681 | Microbiocide, | Inorganic-Silver, |
| polymer | | | | Fungicide, | Heavy metal |
| | | | | Herbicide | |
| Silver zeolite | 130328-18-6 | ##### | NA 125 | Microbiocide | Inorganic-Silver, |
| | | | | | Heavy metal |
| Silver zinc zeolite | 130328-20-0 | ##### | NA 111 | Microbiocide | Inorganic-Silver, |
| | | | | | Inorganic-Zinc, |
| | | | | | Heavy metal |
| Silver, ionic | 14701-21-4 | NA 169 | 5150 | Microbiocide, | Inorganic-Silver, |
| | | | | Fungicide, | Heavy metal |
| | | | | Herbicide | |
| Sodium silver thiosulfate | NA 1398 | NA 197 | NA 196 | Herbicide | Inorganic-Silver, |
| | | | | | Heavy metal |
| 2-Mercaptobenzothiazole, zinc | 155-04-4 | 51705 | 1449 | Fungicide, | Mercaptobenzothiazole, |
| salt | | | | Microbiocide | Inorganic-Zinc |
| 5-Chloro-2- | 53404-93-6 | 51709 | NA 427 | Microbiocide, | Mercaptobenzothiazole, |
| mercaptobenzothiazole, zinc | | | | Fungicide | Inorganic-Zinc |
| salt | | | | | |
| Boric acid(H4B6O11), zinc | 12447-61-9 | ##### | 5094 | Fungicide | Inorganic-Zinc |
| salt(1:2) | | | | | |
| Calcium-zinc LN 193 | NA 653 | NA 120 | 3871 | N/A | Inorganic-Zinc |
| (complex) | | | | | |
| Chromated zinc chloride | NA 1026 | 87801 | 1187 | Insecticide, | Inorganic- |
| | | | | Herbicide, | Chromium(VI), |
| | | | | Microbiocide | Inorganic-Zinc, |
| | | | | | Heavy metal |
| Copper-zinc chromate complex | 1344-74-7 | 21003 | 163 | Fungicide, | Inorganic- |
| | | | | Wood | Chromium(VI), |
| | | | | Preservative | Inorganic-Zinc, |
| | | | | | Heavy metal, |
| | | | | | Inorganic-Copper |
| Copper-zinc sulfate complex | 55072-57-6 | 8102 | 164 | Fungicide | Inorganic-Zinc, |
| | | | | | Inorganic-Copper |
| Copper-zinc sulfate complex, | NA 959 | NA 382 | 1751 | Fungicide | Inorganic-Zinc, |
| monohydrate | | | | | Inorganic-Copper |
| Crag turf fungicide 531 | 12001-20-6 | 21006 | NA 171 | Fungicide | Inorganic-Cadmium, |
| (Cd—Cu—Ca—Zn-Chromate) | | | | | Inorganic- |
| | | | | | Chromium(VI), |
| | | | | | Inorganic-Zinc, |
| | | | | | Heavy metal |
| EDTA, disodium zinc salt | 14025-21-9 | NA 274 | 3189 | N/A | Inorganic-Zinc |
| Lignin sulfonic acid, zinc salt | NA 1014 | NA 102 | 1768 | N/A | Inorganic-Zinc |
| Lignin sulfonic acid, zinc, | NA 1030 | NA 126 | 1771 | N/A | Inorganic-Zinc |
| manganese & iron salts | | | | | |
| Mancozeb | 2233100 | 14504 | 211 | Fungicide | Dithiocarbamate, |
| | | | | | Inorganic-Zinc |
| Metiram | 9006-42-2 | 14601 | 493 | Fungicide | Dithiocarbamate, |
| | | | | | Inorganic-Zinc |
| o-Phenylphenol, alkyl* amine- | 82010-74-0 | 64107 | NA 533 | Microbiocide | Phenols, Inorganic- |
| zinc salt of *(100% C18) | | | | | Zinc |
| Pentachlorophenol, zinc salt | 2917-32-0 | 63008 | NA 510 | Wood | Chlorinated Phenol, |
| | | | | Preservative, | Inorganic-Zinc |
| | | | | Microbiocide, | |
| | | | | Algaecide, | |
| | | | | Fungicide | |
| Polyoxin D zinc salt | 146659-78-1 | ##### | NA 129 | Fungicide | Inorganic-Zinc |
| Propineb | 12071-83-9 | ##### | NA 155 | Fungicide, | Dithiocarbamate, |
| | | | | Microbiocide | Inorganic-Zinc |
| Silver zinc zeolite | 130328-20-0 | ##### | NA 111 | Microbiocide | Inorganic-Silver, |
| | | | | | Inorganic-Zinc, |
| | | | | | Heavy metal |
| Zinc | 7440-66-6 | ##### | 2310 | Herbicide | Inorganic-zinc |
| Zinc 2,4,5-trichlorophenate | 136-24-3 | 64221 | NA 546 | N/A | Chlorinated Phenol, |
| | | | | | Inorganic-Zinc |
| Zinc 2-ethyl hexoate | 136-53-8 | 41203 | 3509 | N/A | Inorganic-Zinc |
| Zinc 2-pyridinethiol-1-oxide | 13463-41-7 | 88002 | 2128 | Microbiocide | Inorganic-Zinc |
| Zinc 8-quinolinolate | 13978-85-3 | 24005 | NA 196 | Fungicide, | Inorganic-Zinc |
| | | | | Microbiocide | |
| Zinc abietate | 6798-76-1 | NA 112 | 3506 | N/A | Inorganic-Zinc |
| Zinc arsenate | 13464-44-3 | 13301 | NA 125 | Herbicide, | Inorganic-Arsenic, |
| | | | | Insecticide, | Heavy metal, |
| | | | | Rodenticide | Inorganic-Zinc |
| Zinc arsenite | 28837-97-0 | 13604 | NA 130 | Herbicide, | Inorganic-Arsenic, |
| | | | | Insecticide, | Inorganic-Zinc, |
| | | | | Rodenticide | Heavy metal |
| Zinc bacitracin | 1405-89-6 | 6309 | NA 87 | Microbiocide | Inorganic-Zinc |
| Zinc carbonate | 3486-35-9 | NA 102 | 3507 | N/A | Inorganic-Zinc |
| Zinc chloride | 7646-85-7 | 87801 | 624 | Microbiocide | Inorganic-Zinc |
| Zinc dehydroabietylammonium | 53404-92-5 | 4211 | NA 75 | Fungicide, | Inorganic-Zinc |
| 2-ethylhexanoate | | | | Microbiocide | |
| Zinc dodecyl benzene sulfonate | 12068-16-5 | NA 112 | 3508 | Microbiocide, | Soap, Inorganic-Zinc |
| | | | | Adjuvant, | |
| | | | | Fungicide, | |
| | | | | Insecticide | |
| Zinc fluosilicate | 16871-71-9 | 75307 | 1019 | Insecticide | Inorganic-Zinc |
| Zinc formate | 557-41-5 | 87802 | NA 838 | N/A | Inorganic-Zinc |
| Zinc hydroxide | 20427-58-1 | 88501 | 3510 | N/A | Inorganic-Zinc |
| Zinc isodecanoate | 30304-30-4 | 41222 | NA 355 | N/A | Inorganic-Zinc |
| Zinc isononanoate | 5398-80-6 | 41212 | NA 353 | N/A | Inorganic-Zinc |
| Zinc isooctanoate | 84082-93-9 | 41205 | NA 351 | N/A | Inorganic-Zinc |
| Zinc mercury chromate | 22323-45-1 | 21004 | NA 170 | Fungicide | Inorganic-Mercury, |
| | | | | | Inorganic- |
| | | | | | chromium(VI), |
| | | | | | Inorganic-Zinc, |
| | | | | | Heavy metal |
| Zinc naphthenate | 12001-85-3 | 88301 | 1111 | Wood | Inorganic-Zinc |
| | | | | Preservative, | |
| | | | | Insecticide, | |
| | | | | Fungicide, Dog | |
| | | | | and Cat | |
| | | | | Repellent | |
| Zinc neodecanoate | 27253-29-8 | 97502 | NA 853 | N/A | Inorganic-Zinc |
| Zinc oxide | 1314-13-2 | 88502 | 666 | Fungicide, | Inorganic-Zinc |
| | | | | Adjuvant | |
| Zinc petroleum (C20-C30) | 68989-17-3 | 88503 | NA 841 | N/A | Inorganic-Zinc |
| sulfonate | | | | | |
| Zinc phenol sulfonate | 127-82-2 | 89002 | NA 842 | N/A | Inorganic-Zinc |
| Zinc phosphide | 1314-84-7 | 88601 | 626 | Rodenticide | Inorganic-Zinc |
| Zinc resinate | 9010-69-9 | 77001 | NA 712 | N/A | Inorganic-Zinc |
| Zinc silicate | NA 1318 | 8103 | NA 176 | Microbiocide | Inorganic-Zinc |
| Zinc stearate | 557-05-1 | 77002 | 3511 | N/A | Inorganic-Zinc |
| Zinc sulfate | 7733-02-0 | 89001 | 667 | Microbiocide, | Inorganic-Zinc |
| | | | | Herbicide | |
| Zinc sulfate, anhydrous | 7733-02-0 | 89001 | 1852 | Microbiocide, | Inorganic-Zinc |
| | | | | Herbicide | |
| Zinc sulfate, basic | 68813-94-5 | 89101 | NA 843 | N/A | Inorganic-Zinc |
| Zinc sulfate, monohydrate | 7446-19-7 | ##### | 2995 | Herbicide | Inorganic-Zinc |
| Zinc trichlorophenate | 30143-22-7 | 64213 | NA 543 | Microbiocide | Chlorinated Phenol, |
| | | | | | Inorganic-Zinc |
| Zineb | 12122-67-7 | 14506 | 627 | Fungicide | Dithiocarbamate, |
| | | | | | Inorganic-Zinc |
| Zineb-ethylene thiuram | NA 1465 | NA 217 | NA 216 | Fungicide | Dithiocarbamate, |
| disulfide adduct | | | | | Inorganic-Zinc |
| Ziram | 137-30-4 | 34805 | 629 | Fungicide, | Dithiocarbamate, |
| | | | | Microbiocide, | Inorganic-Zinc |
| | | | | Dog and Cat | |
| | | | | Repellent | |
| Ziram, cyclohexylamine | 16509-79-8 | 34806 | 1328 | Dog and Cat | Dithiocarbamate, |
| complex | | | | Repellent, | Inorganic-Zinc |
| | | | | Fungicide | |
| 1-butoxy ethoxy-2-propanol | 124-16-3 | NA 113 | 3074 | N/A | Alcohol/Ether |
| 1-butoxy-2-propanol | 5131-86-8 | NA 467 | 3075 | Solvent | Alcohol/Ether |
| 1-decanol | 112-30-1 | 79038 | 1791 | Plant Growth | Alcohol/Ether |
| | | | | Regulator | |
| 1-Heptadecanol | 1454-85-9 | ##### | NA 150 | N/A | Alcohol/Ether |
| 1-heptanol | 111-70-6 | NA 205 | 3222 | N/A | Alcohol/Ether |
| 1-hexanol | 111-27-3 | 79047 | 3229 | N/A | Alcohol/Ether |
| 1-Octanol | 111-87-5 | 79037 | NA 733 | Plant Growth | Alcohol/Ether |
| | | | | Regulator, | |
| | | | | Herbicide | |
| 2,6,8-trimethyl-4-nonanol | 123-17-1 | NA 144 | 3303 | Adjuvant | Alcohol/Ether |
| 2-(2-Butoxyethoxy)ethanol | 112-34-5 | 11502 | NA 117 | Solvent | Alcohol/Ether, |
| | | | | | Glycol Ether |
| 2-butoxyethanol | 111-76-2 | 11501 | 89 | Fungicide, | Alcohol/Ether |
| | | | | Microbiocide, | |
| | | | | Solvent | |
| 2-ethoxyethanol | 110-80-5 | NA 104 | 2554 | Fungicide, | Alcohol/Ether |
| | | | | Microbiocide | |
| 2-Ethyl-1-butanol | 97-95-0 | ##### | NA 148 | N/A | Alcohol/Ether |
| 2-ethyl-1-hexanol | 10-47-6 | NA 251 | 2560 | N/A | Alcohol/Ether |
| 2-Ethyl-1-hexanol | 104-76-7 | 79098 | NA 767 | Solvent | Alcohol/Ether |
| 2-Phenylcyclohexanol | 1444-64-0 | 65002 | NA 550 | N/A | Alcohol/Ether |
| 2-Phenylethanol | 60-12-8 | 1503 | NA 62 | N/A | Alcohol/Ether |
| 3,3,5-trimethyl cyclohexanol | 116-02-9 | NA 982 | 3493 | N/A | Alcohol/Ether |
| 3-phenoxybenzyl alcohol | 13826-35-2 | NA 651 | 2771 | N/A | Alcohol/Ether |
| 4-Methyl-2-pentanol | 108-11-2 | ##### | NA 157 | N/A | Alcohol/Ether |
| Alcohols, C4-C12, normal | NA 392 | NA 713 | 2067 | Solvent, | Alcohol/Ether |
| | | | | Microbiocide | |
| Alcohols, C6-12 | 68603-15-6 | 79029 | 5072 | Microbiocide, | Alcohol/Ether |
| | | | | Solvent | |
| Alcohols, C8-10 | 85566-12-7 | 79059 | 5073 | Microbiocide, | Alcohol/Ether |
| | | | | Solvent | |
| Alkanols (ave. C6), mixed | NA 394 | NA 716 | 1536 | Adjuvant | Alcohol/Ether |
| Alkylaryl polyether alcohol | NA 657 | NA 121 | 878 | Adjuvant | Alcohol/Ether |
| Allyl alcohol | 107-18-6 | 68401 | 3023 | Herbicide | Alcohol/Ether |
| Benzyl alcohol | 100-51-6 | 9502 | 915 | Insecticide, | Alcohol/Ether |
| | | | | Fungicide | |
| Butyl alcohol | 78-92-2 | 1502 | 1482 | Solvent | Alcohol/Ether |
| Cetyl alcohol | 36653-82-4 | 1508 | 2449 | N/A | Alcohol/Ether |
| Cinnamyl alcohol | 104-54-1 | 40512 | NA 343 | Insecticide | Alcohol/Ether |
| Cyclobutaneethanol, 1-methyl- | 30820-22-5 | ##### | NA 922 | N/A | Alcohol/Ether |
| 2-(1-methylethenyl)-,cis- | | | | | |
| Cyclohexanol | 108-93-0 | 25904 | 3121 | Solvent | Alcohol/Ether |
| Denatured ethanol | NA 1033 | NA 129 | 2102 | Solvent | Alcohol/Ether |
| Dimethyl ether | 115-10-6 | NA 132 | 2517 | Solvent | Alcohol/Ether |
| Ethyl alcohol | 64-17-5 | 1501 | 8 | Microbiocide, | Alcohol/Ether |
| | | | | Solvent, | |
| | | | | Adjuvant, | |
| | | | | Molluscicide | |
| Ethylene oxide | 75-21-8 | 42301 | 277 | Fumigant | Alcohol/Ether |
| Fatty alcohols (100% C4-C10) | NA 126 | NA 241 | 3654 | Adjuvant | Alcohol/Ether |
| Fatty alcohols (55.10% C10, | NA 1265 | 79089 | NA 173 | Plant Growth | Alcohol/Ether |
| 42.88% C8, 1.01% C6, 1.01% | | | | Regulator, | |
| C12) | | | | Adjuvant | |
| Fatty alcohols (65% C12, 26% | 67762-41-8 | 79056 | NA 744 | Adjuvant | Alcohol/Ether |
| C14, 8% C16, 1% C10) | | | | | |
| Hydrocinnamyl alcohol | 122-97-4 | 40511 | NA 342 | N/A | Alcohol/Ether |
| i-Nonyl alcohol | 3452-97-9 | ##### | NA 153 | N/A | Alcohol/Ether |
| Isobutyl alcohol | 78-83-1 | 1507 | 2622 | Solvent | Alcohol/Ether |
| Isodecyl alcohol | 25339-17-7 | NA 168 | 3242 | Solvent | Alcohol/Ether |
| Isopropyl alcohol | 67-63-0 | 47501 | 342 | Microbiocide, | Alcohol/Ether |
| | | | | Solvent | |
| Isostearyl alcohol | 27458-93-1 | NA 156 | 2627 | Adjuvant | Alcohol/Ether |
| Lauryl alcohol | 112-53-8 | 1509 | 2343 | Pheromone, | Pheromone, |
| | | | | Adjuvant | Alcohol/Ether |
| Methanol | 67-56-1 | 53801 | 1018 | Solvent, | Alcohol/Ether |
| | | | | Adjuvant, | |
| | | | | Microbiocide | |
| Methoxypropanol | 107-98-2 | NA 90 | 2675 | Solvent | Alcohol/Ether |
| Myristyl alcohol | 112-72-1 | 1510 | 2344 | Pheromone, | Alcohol/Ether |
| | | | | Fragrance | |
| n-Butyl alcohol | 71-36-3 | NA 468 | 3077 | Solvent | Alcohol/Ether |
| n-Decyl alcohol | 112-30-1 | 79038 | 3124 | Plant Growth | Alcohol/Ether |
| | | | | Regulator | |
| n-Propoxy propanol | 30136-13-1 | NA 798 | 2837 | Solvent | Alcohol/Ether |
| n-Propyl alcohol | 71-23-8 | 47502 | 1603 | Solvent | Alcohol/Ether |
| Oleyl alcohol | 143-28-2 | NA 9 | 3313 | Adjuvant | Alcohol/Ether |
| Propargyl alcohol | 107-19-7 | 68702 | NA 609 | Breakdown | Alcohol/Ether |
| | | | | product | |
| Propylene oxide | 75-56-9 | 42501 | 508 | Fumigant | Alcohol/Ether |
| sec-Butanol | 75-65-0 | 1505 | NA 64 | Solvent | Alcohol/Ether |
| Sec-butyl alcohol | 78-92-2 | 1502 | 3078 | Solvent | Alcohol/Ether |
| Stearyl alcohol | 112-92-5 | NA 110 | 2892 | Adjuvant | Alcohol/Ether |
| Tetrahydro furfuryl alcohol | 97-99-4 | NA 945 | 3471 | Solvent | Alcohol/Ether |
| (2- | 150-39-0 | 39108 | NA 330 | Adjuvant | Chelating agent |
| Hydroxyethyl)ethylenediamine | | | | | |
| triacetic acid | | | | | |
| Dimethamine EDTA | NA 167 | NA 319 | 3159 | Adjuvant | Chelating agent |
| EDTA | 60-00-4 | 39101 | 1702 | Adjuvant | Chelating agent |
| EDTA, ammonium salt | 7379-26-2 | 39117 | 889 | Adjuvant | Chelating agent |
| EDTA, diethanolamine salt | 68133-37-9 | NA 273 | 3187 | Adjuvant | Chelating agent |
| EDTA, disodium salt | 139-33-3 | 39115 | 2101 | Herbicide, | Chelating agent |
| | | | | Adjuvant | |
| EDTA, disodium salt | 139-33-3 | 39115 | 954 | Herbicide, | Chelating agent |
| | | | | Adjuvant | |
| EDTA, iron chelate | NA 890 | NA 170 | 3240 | Herbicide | Chelating agent |
| EDTA, monoethanolamine salt | 7379-26-2 | 39117 | 2013 | Adjuvant | Chelating agent |
| EDTA, monopotassium salt | 7379-27-3 | 39111 | NA 331 | Adjuvant | Chelating agent |
| EDTA, potassium salt | NA 891 | NA 170 | 3350 | Adjuvant | Chelating agent |
| EDTA, sodium salt | 17421-79-3 | 39103 | 1757 | Adjuvant | Chelating agent |
| EDTA, tetrapotassium salt | 5964-35-2 | 39118 | 1054 | Adjuvant | Chelating agent |
| EDTA, tetrasodium salt | 64-02-8 | 39107 | 759 | Adjuvant | Chelating agent |
| EDTA, trisodium salt | 150-38-9 | 39110 | 953 | Adjuvant | Chelating agent |
| Ethanolamine | 63517-71-5 | 39116 | NA 334 | Adjuvant, | Chelating agent |
| ethylenediaminetetraacetate | | | | Fungicide | |
| N-(2-hydroxyethyl) ethylene | 139-89-9 | 39109 | 911 | Microbiocide, | Chelating agent |
| diamine triacetic acid, trisodium | | | | Adjuvant | |
| salt | | | | | |
| Nitrilotriacetic acid | 139-13-9 | NA 228 | NA 228 | Adjuvant | Chelating agent |
| Nitrilotriacetic acid, sodium | 10042-84-9 | ##### | NA 121 | Adjuvant | Chelating agent |
| salt | | | | | |
| Sodium N-(2- | 53404-54-9 | 39121 | NA 335 | Adjuvant | Chelating agent |
| hydroxyethyl)ethylenediaminetriacetate | | | | | |
| Tetra(monoethanolamine) | 53404-52-7 | 39112 | NA 332 | Adjuvant, | Chelating agent |
| ethylenediaminetetraacetate | | | | Adjuvant | |
| Triethylamine EDTA | NA 790 | NA 146 | 3486 | Adjuvant | Chelating agent |
| Triethylamine nitrilo triacetate | NA 537 | NA 978 | 3487 | Adjuvant | Chelating agent |
| Tripotassium | 17572-97-3 | 39113 | 1177 | Adjuvant | Chelating agent |
| ethylenediaminetetraacetate | | | | | |
| Trisodium nitrilo triacetate | 5064-31-3 | 39106 | 1121 | Adjuvant | Chelating agent |
| 2,4,5-T, butoxyethanol ester | 2545-59-7 | 82053 | 819 | Herbicide | Chlorophenoxy acid |
| | | | | | or ester, Glycol Ether |
| 2,4-D, butoxyethanol ester | 1929-73-3 | 30053 | 802 | Herbicide | Chlorophenoxy acid |
| | | | | | or ester, Glycol Ether |
| 2-(2-Butoxyethoxy)ethanol | 112-34-5 | 11502 | NA 117 | Solvent | Alcohol/Ether, |
| | | | | | Glycol Ether |
| 4-(2,4-DB), butoxyethanol | 32357-46-3 | 30853 | 837 | Herbicide | Chlorophenoxy acid |
| ester | | | | | or ester, Glycol Ether |
| Alkylaryl polyglycol ether | NA 319 | NA 599 | 1264 | Adjuvant | Polyalkyloxy |
| | | | | | Compound, Glycol |
| | | | | | Ether |
| Cinoxate | 104-28-9 | 76604 | NA 706 | N/A | Glycol Ether |
| Dichlorprop, butoxyethanol | 53404-31-2 | 31453 | 923 | Plant Growth | Chlorophenoxy acid |
| ester | | | | Regulator, | or ester, Glycol Ether |
| | | | | Herbicide | |
| Diethylene glycol monoethyl | 111-90-0 | 11504 | 2505 | Adjuvant, | Glycol Ether |
| ether | | | | Solvent | |
| Diethylene glycol monomethyl | 111-77-3 | 42204 | 2506 | Adjuvant, | Glycol Ether |
| ether | | | | Solvent | |
| Ethoxyethoxyethyl 2,4- | 53404-36-7 | 30058 | NA 245 | Herbicide | Chlorophenoxy acid |
| dichlorophenoxyacetate | | | | | or ester, Glycol Ether |
| Ethyl amino benzoate | 111-15-9 | NA 260 | 3192 | N/A | Glycol Ether |
| Ethylene glycol, mono-butyl | 111-76-2 | 11501 | 1880 | Solvent, | Glycol Ether |
| ether | | | | Adjuvant | |
| Haloxyfop-etotyl (unstated | 87237-48-7 | NA 200 | NA 199 | Herbicide | Aryloxyphenoxy |
| stereochemistry) | | | | | propionic acid, |
| | | | | | Glycol Ether |
| MCPA, butoxyethanol ester | 19480-43-4 | 30553 | 785 | Herbicide | Chlorophenoxy acid |
| | | | | | or ester, Glycol Ether |
| Silvex, butoxyethanol ester | 19398-13-1 | 82553 | 828 | Herbicide | Chlorophenoxy acid |
| | | | | | or ester, Glycol Ether |
| Triclopyr, butoxyethyl ester | 64700-56-7 | ##### | 2170 | Herbicide | Chloropyridinyl, |
| | | | | | Glycol Ether |
| (Decyl oxy) poly (oxy | 9038-29-3 | NA 117 | 3636 | Adjuvant | Polyalkyloxy |
| ethylene) poly (oxy propylene) | | | | | Compound |
| (Octyl oxy) poly (oxyethylene) | 61827-84-7 | NA 102 | 3707 | Adjuvant | Polyalkyloxy |
| poly (oxypropylene) | | | | | Compound |
| 2,6,8-trimethyl-4-nonyloxy | NA 1162 | NA 175 | 5599 | Adjuvant | Polyalkyloxy |
| polyethylene oxyethanol | | | | | Compound |
| 4-nonyl phenoxy poly | NA 805 | NA 149 | 3698 | Soap/Surfactant, | Polyalkyloxy |
| (ethylene oxy) ethyl phosphate, | | | | Adjuvant | Compound |
| magnesium salt | | | | | |
| a-(p-nonylphenyl)-omega- | NA 27 | NA 40 | 2066 | Microbiocide | Polyalkyloxy |
| hydroxypoly (oxyethylene) | | | | | Compound |
| with an average of 4.2 moles of | | | | | |
| ethylene oxide.the nonly group | | | | | |
| is a propylene | | | | | |
| a-(p-nonylphenyl)-omega- | NA 28 | NA 41 | 2052 | Microbiocide | Polyalkyloxy |
| hydroxypoly (oxyethylene) | | | | | Compound |
| with an averageof 9-10 moles | | | | | |
| of ethylene oxide | | | | | |
| Alkenoic acid poly oxyalkylene | NA 396 | NA 718 | 2364 | Adjuvant | Polyalkyloxy |
| ether & alkyl poly oxyalkylene | | | | | Compound |
| ether | | | | | |
| Alkoxy poly (ethyleneoxy) | NA 286 | NA 555 | 1390 | Adjuvant, | Polyalkyloxy |
| ethyl phosphate | | | | Soap/Surfactant | Compound |
| Alkyl (100% C10-C13) oxy | NA 288 | NA 560 | 3575 | Adjuvant, | Polyalkyloxy |
| poly (ethylene oxy) ethyl | | | | Soap/Surfactant | Compound |
| phosphate, mono- | | | | | |
| Alkyl (100% C10-C14) oxy | 68585-36-4 | NA 579 | 3574 | Adjuvant, | Polyalkyloxy |
| poly (ethylene oxy) ethyl | | | | Soap/Surfactant | Compound |
| phosphate | | | | | |
| Alkyl (100% C11-C15) | 68131-40-8 | 79084 | 3572 | Insecticide, | Polyalkyloxy |
| phenoxypoly (ethylene oxy) | | | | Fungicide, | Compound |
| ethanol | | | | Avicide, | |
| | | | | Adjuvant | |
| Alkyl (C8,C10) polyglycoside | 68515-73-1 | NA 154 | 4015 | Adjuvant | Polyalkyloxy |
| | | | | | Compound |
| Alkyl and alkylaryl poly | NA 398 | NA 720 | 1706 | Adjuvant | Polyalkyloxy |
| (oxyethylene) glycols, mixed | | | | | Compound |
| Alkyl aryl alkoxylate | NA 314 | NA 594 | 2366 | Adjuvant | Polyalkyloxy |
| | | | | | Compound |
| Alkyl aryl polyoxyalkane ether | NA 323 | NA 603 | 2370 | Adjuvant | Polyalkyloxy |
| and free fatty acids | | | | | Compound |
| Alkyl oxy poly (ethyleneoxy) | NA 679 | NA 125 | 1715 | Adjuvant, | Polyalkyloxy |
| ethyl phosphate | | | | Soap/Surfactant | Compound |
| Alkyl oxy polyethoxy ethanol | NA 306 | NA 578 | 2048 | Adjuvant | Polyalkyloxy |
| | | | | | Compound |
| Alkyl oxy-polyoxyethylene and | NA 289 | NA 561 | 2136 | Adjuvant | Polyalkyloxy |
| alkyl phenyloxy- | | | | | Compound |
| polyoxyethylene | | | | | |
| Alkyl phenoxypoly (ethoxy) | NA 291 | NA 563 | 1173 | Adjuvant | Polyalkyloxy |
| ethanol | | | | | Compound |
| Alkyl phenyl poly (ethoxy) | NA 294 | NA 566 | 1064 | Adjuvant | Polyalkyloxy |
| ethanol | | | | | Compound |
| Alkyl polyethylene glycol ether | NA 668 | NA 123 | 1376 | Adjuvant, | Polyalkyloxy |
| | | | | Soap/Surfactant | Compound |
| Alkyl polyoxy alkylene ether | NA 295 | NA 567 | 2123 | Adjuvant | Polyalkyloxy |
| | | | | | Compound |
| Alkyl polyoxy ethylene ether, | NA 592 | NA 107 | 2014 | Adjuvant | Polyalkyloxy |
| diphosphoric acid ester | | | | | Compound |
| Alkyl polyoxy ethylene ether, | NA 296 | NA 568 | 2377 | Adjuvant | Polyalkyloxy |
| free fatty acids | | | | | Compound |
| Alkyl polyoxy ethylene ether, | NA 694 | NA 127 | 2015 | Adjuvant | Polyalkyloxy |
| monophosphoric acid ester | | | | | Compound |
| Alkyl polyoxy ethylene ethers, | NA 297 | NA 569 | 2158 | Adjuvant | Polyalkyloxy |
| polymerized resins and fatty | | | | | Compound |
| acids | | | | | |
| Alkyl polyoxy ethylene glycols | NA 285 | NA 554 | 2051 | Adjuvant | Polyalkyloxy |
| | | | | | Compound |
| Alkylaryl hydroxypoly | NA 316 | NA 596 | 2037 | Adjuvant | Polyalkyloxy |
| (oxyethylene) ethanol | | | | | Compound |
| Alkylaryl poly(oxyethylene) | NA 315 | NA 595 | 748 | Adjuvant | Polyalkyloxy |
| glycol | | | | | Compound |
| Alkylaryl polyalkoxylated | NA 705 | NA 129 | 2174 | Adjuvant | Polyalkyloxy |
| alcohols | | | | | Compound |
| Alkylaryl polyethoxy ethanol | NA 714 | NA 130 | 2368 | Adjuvant, | Polyalkyloxy |
| phosphate | | | | Soap/Surfactant | Compound |
| Alkylaryl polyethoxyethanol | NA 912 | NA 174 | 5323 | Adjuvant | Polyalkyloxy |
| sulfates | | | | | Compound |
| Alkylaryl polyethylene glycol | NA 318 | NA 598 | 2198 | Adjuvant, | Polyalkyloxy |
| ether | | | | Soap/Surfactant | Compound |
| Alkylaryl polyglycol ester | NA 1470 | NA 218 | NA 218 | Adjuvant | Polyalkyloxy |
| | | | | | Compound |
| Alkylaryl polyglycol ether | NA 319 | NA 599 | 1264 | Adjuvant | Polyalkyloxy |
| | | | | | Compound, Glycol |
| | | | | | Ether |
| Alkylaryl polyoxy glycol | NA 322 | NA 602 | 2369 | Adjuvant | Polyalkyloxy |
| | | | | | Compound |
| Alkylaryl polyoxyethylene | NA 320 | NA 600 | 881 | Adjuvant | Polyalkyloxy |
| ether | | | | | Compound |
| Alkylaryl polyoxyethylene | NA 321 | NA 601 | 2024 | Adjuvant, | Polyalkyloxy |
| glycol phosphate ester | | | | Soap/Surfactant | Compound |
| Alkylaryl poyloxyethylene | NA 671 | NA 124 | 1507 | Adjuvant | Polyalkyloxy |
| ethanol | | | | | Compound |
| Alkylpoly glycol ether | NA 292 | NA 564 | 1172 | Adjuvant | Polyalkyloxy |
| | | | | | Compound |
| Allyl oxy poly ethylene glycol | NA 279 | NA 545 | 2376 | N/A | Polyalkyloxy |
| monoallyl acetate | | | | | Compound |
| Alpha (para-tert-butyl phenyl)- | NA 235 | NA 463 | 1605 | Adjuvant | Polyalkyloxy |
| omega-hydroxypoly | | | | | Compound |
| (oxyethylene)- | | | | | |
| Alpha (para-tert-butylphenyl)- | NA 234 | NA 462 | 1786 | Adjuvant | Polyalkyloxy |
| omega-hydroxypoly | | | | | Compound |
| (oxyethylene) | | | | | |
| Alpha-(omega-sec-butyl | NA 231 | NA 452 | 1885 | Adjuvant | Polyalkyloxy |
| phenylpoly (oxypropylene))- | | | | | Compound |
| omega-hydroxypoly | | | | | |
| Alpha-(p-(1,1,3,3-tetramethyl | NA 609 | NA 111 | 1932 | Adjuvant | Polyalkyloxy |
| butyl) phenyl)-omega-hydroxypoly | | | | | Compound |
| Alpha-(p-nonylphenyl)-omega- | NA 745 | NA 135 | 2710 | Adjuvant | Polyalkyloxy |
| hydroxypoly (oxypropylene) | | | | | Compound |
| Alpha-(para-nonyl phenyl)- | NA 18 | NA 31 | 1649 | Adjuvant | Polyalkyloxy |
| omega-hydroxypoly | | | | | Compound |
| (oxyethylene) | | | | | |
| Alpha-alkyl (43% C10, | NA 684 | NA 126 | 1834 | Microbiocide | Polyalkyloxy |
| 30% C14, 12% C12, 10% C16, | | | | | Compound |
| 5% C18) poly (oxyethylene) | | | | | |
| poly (oxypropylene) - I2 | | | | | |
| complex | | | | | |
| Alpha-alkyl (C10-C12)-omega- | NA 307 | NA 582 | 2077 | Adjuvant | Polyalkyloxy |
| hydroxypoly (oxyethylene) | | | | | Compound |
| Alpha-alkyl (C10-C18)- | NA 301 | NA 573 | 1700 | Adjuvant | Polyalkyloxy |
| omega-hydroxypoly | | | | | Compound |
| (oxyethylene) sulfate | | | | | |
| 150 solvent neutral BP 901 | NA 747 | NA 136 | 2724 | Insecticide | Petroleum derivative |
| base oil | | | | | |
| Alkylated aromatic petroleum | 68919-17-5 | 54001 | NA 451 | Insecticide, | Petroleum derivative |
| oil | | | | Adjuvant | |
| Amoco 6342 | NA 715 | NA 131 | 2384 | N/A | Petroleum derivative |
| Anthracene oil | 120-12-7 | NA 185 | NA 184 | Insecticide, | Petroleum derivative |
| | | | | Herbicide, | |
| | | | | Rodenticide | |
| Aromatic 100 | NA 269 | NA 524 | 2394 | Insecticide, | Petroleum derivative |
| | | | | Solvent | |
| Aromatic 150 | NA 716 | NA 131 | 2395 | Insecticide, | Petroleum derivative |
| | | | | Solvent | |
| Aromatic 200 | 64742-94-5 | 6602 | 2396 | Insecticide, | Petroleum derivative |
| | | | | Solvent | |
| Asphalt solids | 8052-42-4 | 22001 | 1140 | Pruning Aid | Petroleum derivative |
| Bitumen | 8052-42-4 | 22002 | NA 171 | N/A | Petroleum derivative |
| Candle wax | NA 227 | NA 440 | 2443 | N/A | Petroleum derivative |
| Chevron 100 neutral oil | 68602-80-2 | 6601 | 2456 | Insecticide | Petroleum derivative |
| Chevron base oil “C” | NA 224 | NA 431 | 2455 | Insecticide | Petroleum derivative |
| Chevron solvent 425 | NA 722 | NA 132 | 2457 | Solvent | Petroleum derivative |
| Coal tar distillate boiling | 65996-91-0 | 6101 | 5042 | Insecticide, | Petroleum derivative |
| between 270-300 deg C. | | | | Herbicide | |
| Coal tar hydrocarbons | 8007-45-2 | 22003 | 1719 | Wood | Petroleum derivative |
| | | | | Preservative | |
| Coal tar neutral oils and coal | 65996-82-9 | 25001 | 144 | Insecticide, | Petroleum derivative |
| tar acid combinations | | | | Herbicide | |
| Coal tar phenols | 1319-77-3 | 22101 | 1720 | N/A | Petroleum derivative, |
| | | | | | Phenols |
| Coal tar pitch >351 | 65996-93-2 | ##### | NA 106 | Insecticide | Petroleum derivative |
| deg. C.(AWPI) | | | | | |
| Conoco LPA | NA 195 | NA 386 | 2478 | N/A | Petroleum derivative |
| Creosote | 8001-58-9 | 25004 | 171 | Wood | Petroleum derivative |
| | | | | Preservative | |
| Creosote oil (note: derived | 61789-28-4 | 25003 | NA 203 | Wood | Petroleum derivative |
| from any source) | | | | Preservative, | |
| | | | | Fungicide | |
| Cumene | 98-82-8 | NA 105 | 3116 | N/A | Petroleum derivative |
| Cyclohexane | 110-82-7 | 25901 | 2087 | Solvent | Petroleum derivative |
| Diesel fuel no. 2 | 68476-34-6 | 63514 | 3923 | Insecticide, | Petroleum derivative |
| | | | | Adjuvant | |
| Ethyl benzene | 100-41-4 | NA 142 | 3197 | N/A | Petroleum derivative |
| Ethylene | 74-85-1 | 41901 | 270 | Plant Growth | Petroleum derivative |
| | | | | Regulator | |
| Fuel oil #1 | 70892-10-3 | 63511 | NA 520 | N/A | Petroleum derivative |
| Fuel oil #2 | 68476-30-2 | 63505 | NA 517 | N/A | Petroleum derivative |
| Fuel oil #4 | 68476-31-3 | 63512 | NA 521 | N/A | Petroleum derivative |
| Fuel oil #6 | 68553-00-4 | 63513 | NA 522 | N/A | Petroleum derivative |
| Gasoline | NA 945 | NA 221 | 1899 | N/A | Petroleum derivative |
| Heavy oil (coal) 301-350 deg. C. | 90640-86-1 | ##### | NA 106 | N/A | Petroleum derivative |
| (AWPI) | | | | | |
| Hydrotreated paraffinic solvent | NA 91 | NA 182 | 2601 | Solvent | Petroleum derivative |
| Isobutane | 75-28-5 | 97101 | 2621 | Propellant | Petroleum derivative |
| Isoparaffin, C11-C12 | NA 85 | NA 163 | 2623 | N/A | Petroleum derivative |
| Isoparaffin, C12-C14 | NA 86 | NA 164 | 2624 | N/A | Petroleum derivative |
| Isoparaffinic hydrocarbons | 64771-72-8 | ##### | 1641 | N/A | Petroleum derivative |
| Isopentane | 78-78-4 | NA 165 | 3244 | Solvent | Petroleum derivative |
| Jet fuel | 94114-58-6 | 63515 | NA 523 | Insecticide, | Petroleum derivative |
| | | | | Solvent | |
| Kerosene | 8008-20-6 | 63501 | 2071 | Insecticide | Petroleum derivative |
| KM spray oil | NA 78 | NA 150 | 2631 | N/A | Petroleum derivative |
| Ligroin | 8032-32-4 | 63506 | 2642 | Solvent | Petroleum derivative |
| Low molecular weight | 64642-47-8 | NA 173 | 5252 | Insecticide | Petroleum derivative |
| paraffinic oil | | | | | |
| MCIAL code 401 | 64742-55-8 | 63503 | 2045 | Insecticide, | Petroleum derivative |
| | | | | Adjuvant | |
| Mineral oil | 8012-95-1 | 63502 | 401 | Insecticide, | Petroleum derivative |
| | | | | Adjuvant | |
| Mineral oil, petroleum | 64741-89-5 | 63503 | 3846 | Herbicide, | Petroleum derivative |
| distillates, solvent refined light | | | | Plant Growth | |
| | | | | Regulator, | |
| | | | | Insecticide, | |
| | | | | Adjuvant | |
| Mineral oil, petroleum extract, | 64724-04-7 | NA 171 | 3847 | Insecticide, | Petroleum derivative |
| heavy paraffinic distillate | | | | Adjuvant | |
| Mineral seal oil | NA 930 | NA 67 | 2687 | Insecticide, | Petroleum derivative |
| | | | | Adjuvant | |
| Mineral spirits | 8032-32-4 | 63506 | 2688 | Insecticide, | Petroleum derivative |
| | | | | Solvent, | |
| | | | | Fungicide | |
| n-Hexane | 110-54-3 | NA 103 | 3227 | Solvent | Petroleum derivative |
| Naphtha, heavy aromatic | 64742-94-5 | 6602 | 1717 | Insecticide, | Petroleum derivative |
| | | | | Solvent | |
| Orchex 796 oil | NA 899 | NA 172 | 5179 | Insecticide, | Petroleum derivative |
| | | | | Adjuvant | |
| Paraffin wax | 8002-74-2 | NA 107 | 1214 | Rodenticide | Petroleum derivative |
| Paraffin waxes (petroleum) | 64742-51-4 | NA 694 | 3545 | N/A | Petroleum derivative |
| hydrotreated | | | | | |
| Paraffin waxes and | 61788-76-9 | NA 693 | 3721 | N/A | Petroleum derivative |
| hydrocarbon waxes, | | | | | |
| chlorinated | | | | | |
| Pentane | 109-66-0 | 98001 | 469 | Solvent | Petroleum derivative |
| Petroleum derivative resin | 64742-16-1 | 11401 | 1411 | N/A | Petroleum derivative |
| Petroleum distillates | 2227378 | 63503 | 763 | Insecticide | Petroleum derivative |
| Petroleum distillates, aliphatic | 2227378 | 63503 | 2768 | Insecticide, | Petroleum derivative |
| | | | | Adjuvant, | |
| | | | | Solvent | |
| Petroleum distillates, aromatic | 68477-31-6 | 6501 | 1814 | Solvent, | Petroleum derivative |
| | | | | Herbicide, | |
| | | | | Insecticide | |
| Petroleum distillates, refined | 64741-51-1 | NA 654 | 2106 | Insecticide | Petroleum derivative |
| Petroleum hydrocarbons | 8012-95-1 | 63502 | 473 | Insecticide, | Petroleum derivative |
| | | | | Solvent | |
| Petroleum jelly | 2229873 | ##### | 1443 | Insect | Petroleum derivative |
| | | | | Repellent, | |
| | | | | Adjuvant | |
| Petroleum naphthenic oils | NA 990 | NA 655 | 2107 | Insecticide, | Petroleum derivative |
| | | | | Adjuvant | |
| Petroleum oil, unclassified | 68815-10-1 | NA 121 | 765 | Insecticide, | Petroleum derivative |
| | | | | Herbicide, | |
| | | | | Fungicide, | |
| | | | | Adjuvant | |
| Petroleum sulfonates | 61789-85-3 | ##### | 766 | N/A | Petroleum derivative |
| Petroleum, unrefined | 2227378 | 63503 | 1730 | Insecticide, | Petroleum derivative |
| | | | | Adjuvant | |
| Polybutenes | 9003-29-6 | 11402 | 1040 | Rodenticide | Petroleum derivative |
| Polyisobutylene | 9003-27-4 | 11403 | 1561 | Rodenticide | Petroleum derivative |
| Propane | 74-98-6 | NA 109 | 2832 | Propellant | Petroleum derivative |
| Propylene | 1150-70-1 | NA 800 | 2838 | N/A | Petroleum derivative |
| Solvent naphtha (coal), 150-200 | 65996-79-4 | ##### | NA 106 | Solvent | Petroleum derivative |
| deg. C. | | | | | |
| Solvent naphtha (petroleum), | 64742-95-6 | 86803 | 3804 | Solvent | Petroleum derivative |
| light aromatic | | | | | |
| Summer oil | NA 1468 | NA 218 | NA 218 | Insecticide, | Petroleum derivative |
| | | | | Adjuvant | |
| Sun superior spray oil 7N | NA 769 | NA 139 | 2902 | Insecticide, | Petroleum derivative |
| | | | | Adjuvant | |
| Synthetic beeswax | NA 509 | NA 920 | 2910 | N/A | Petroleum derivative |
| Tar | NA 1312 | 22004 | NA 176 | Wood | Petroleum derivative |
| | | | | Preservative | |
| Tar acid oil | NA 1041 | NA 139 | 2914 | Insecticide | Petroleum derivative |
| Toluene | 108-88-3 | 80601 | 1281 | Solvent | Petroleum derivative |
| Turpentine | 8006-64-2 | 84501 | 605 | Insecticide, | Petroleum derivative |
| | | | | Adjuvant | |
| White mineral oil | 8042-47-5 | 63510 | NA 519 | Insecticide, | Petroleum derivative |
| | | | | Adjuvant | |
| Winter oil | NA 1472 | NA 219 | NA 218 | Adjuvant | Petroleum derivative |
| Xylene | 1330-20-7 | 86802 | 622 | Solvent, | Petroleum derivative |
| | | | | Microbiocide | |
| Xylene range aromatic solvent | 68920-06-9 | NA 102 | 862 | Solvent, | Petroleum derivative |
| | | | | Insecticide | |
| 3-(3-hydroxypropyl)-heptamethyl | NA 90 | NA 178 | 2610 | Adjuvant | Silicone |
| trisiloxane, ethoxylated, | | | | | |
| acetate | | | | | |
| Compounded silicone | NA 703 | NA 129 | 2124 | Adjuvant | Silicone |
| Dimethyl poly sloxane | 63148-62-9 | NA 104 | 1861 | Insecticide, | Silicone |
| | | | | Adjuvant | |
| Dimethyl silicone fluid | NA 696 | NA 128 | 2028 | Adjuvant | Silicone |
| emulsion | | | | | |
| Dow Corning antifoam Y-30 | NA 146 | NA 280 | 2539 | Adjuvant | Silicone |
| emulsion | | | | | |
| Dow Corning DB-110A- | NA 577 | NA 104 | 2540 | Adjuvant | Silicone |
| antifoam emulsion | | | | | |
| Heptamethyltrisiloxane | 67674-67-3 | NA 153 | 4009 | Adjuvant | Silicone |
| ethoxylated (8 EO) | | | | | |
| Methyl silicone resins | 9016-00-6 | NA 77 | 3689 | Adjuvant | Silicone |
| Methylated silica | 67762-90-7 | NA 173 | 5292 | N/A | Silicone |
| Organo/modified polysiloxane | 37281-78-0 | NA 153 | 3997 | Adjuvant | Silicone |
| Organosilicone, polyoxyalkylene | 67762-85-0 | NA 173 | 5231 | Adjuvant | Silicone |
| ether copolymer | | | | | |
| Polyalkene oxide modified | 27306-78-1 | NA 146 | 3520 | Adjuvant | Silicone |
| heptamethyl trisiloxane | | | | | |
| Polyalkyleneoxide modified | NA 904 | NA 172 | 5203 | Adjuvant | Silicone |
| polydimethyl-siloxane | | | | | |
| Polyether modified | NA 906 | NA 172 | 5223 | Adjuvant | Silicone |
| polysiloxane | | | | | |
| Polysiloxane | NA 1337 | NA 180 | 5718 | Adjuvant | Silicone |
| Silicone | NA 815 | NA 150 | 3796 | Adjuvant | Silicone |
| Silicone defoamer | NA 462 | NA 826 | 1917 | Adjuvant | Silicone |
| Silicone mold release agent SM | NA 463 | NA 827 | 2854 | N/A | Silicone |
| 2140 | | | | | |
| Silicone-polyether copolymer | 27306-78-1 | NA 151 | 3841 | Adjuvant | Silicone |
| Trimethyl siloxy stearate | NA 541 | NA 987 | 2965 | N/A | Silicone |
| 1,1,2,3-tetramethyl butylamine | NA 1010 | NA 948 | 3472 | Microbiocide, | Soap |
| dodecyl benzene sulfonate | | | | Adjuvant, | |
| | | | | Fungicide, | |
| | | | | Insecticide | |
| 1,3-propane diamine dodecyl | NA 1021 | NA 115 | 3369 | Microbiocide, | Soap |
| benzene sulfonate | | | | Adjuvant, | |
| | | | | Fungicide, | |
| | | | | Insecticide | |
| 2,2-oxybis (4-dodecyl benzene | NA 1161 | NA 175 | 1741 | Microbiocide, | Soap |
| sulfonate) | | | | Adjuvant, | |
| | | | | Fungicide, | |
| | | | | Insecticide | |
| 2-((2-amino ethyl) amino) | 68084-55-9 | NA 538 | 3034 | Microbiocide, | Soap |
| ethanol dodecyl benzene | | | | Adjuvant, | |
| sulfonate | | | | Fungicide, | |
| | | | | Insecticide | |
| Alkanolamide surfactants | NA 911 | NA 174 | 5322 | Soap/Surfactant, | Soap |
| | | | | Adjuvant | |
| Alkyl* sodium benzene | 68411-30-3 | ##### | NA 126 | Microbiocide, | Soap |
| sulfonate *(56% C11, 33% C12, | | | | Adjuvant, | |
| 7% C10, 4% C13) | | | | Fungicide, | |
| | | | | Insecticide | |
| Ammonium lauryl sulfate | 2235-54-3 | 79028 | 1017 | Adjuvant, | Soap |
| | | | | Insecticide, | |
| | | | | Microbiocide, | |
| | | | | Fungicide | |
| Ammonium oleate | 544-60-5 | 31703 | 1230 | Adjuvant, | Soap |
| | | | | Insecticide | |
| Ammonium tall oil fatty acid | 544-60-5 | 31703 | 995 | Adjuvant, | Soap |
| soap | | | | Insecticide | |
| Benzene, 1,1′-oxybis- | 119345-04-9 | NA 147 | 3561 | Microbiocide, | Soap |
| tetrapropylene derivatives, | | | | Adjuvant, | |
| sulfonated, sodium | | | | Fungicide, | |
| | | | | Insecticide | |
| Butylamine dodecyl benzene | 12068-09-6 | NA 469 | 3079 | Microbiocide, | Soap |
| sulfonate | | | | Adjuvant, | |
| | | | | Fungicide, | |
| | | | | Insecticide | |
| Coconut oil soap | NA 199 | NA 391 | 2088 | Adjuvant, | Soap |
| | | | | Insecticide, | |
| | | | | Soap/Surfactant | |
| DEA-dodecyl benzene | 26545-53-9 | 79015 | 3123 | Microbiocide, | Soap |
| sulfonate | | | | Adjuvant, | |
| | | | | Fungicide, | |
| | | | | Insecticide | |
| Diethylamine salt of coconut | NA 210 | NA 402 | 1066 | Adjuvant, | Soap |
| fatty acid | | | | Soap/Surfactant | |
| Diisopropylamine dodecyl | 29061-61-8 | NA 327 | 3156 | Microbiocide, | Soap |
| benzene sulfonate | | | | Adjuvant, | |
| | | | | Fungicide, | |
| | | | | Insecticide | |
| Dimethylalmine propylamine | NA 953 | NA 318 | 3162 | Microbiocide, | Soap |
| tridecyl benzene sulfonate | | | | Adjuvant, | |
| | | | | Fungicide, | |
| | | | | Insecticide | |
| Dimethylamine dodecyl | NA 954 | NA 324 | 3161 | Microbiocide, | Soap |
| benzene sulfonate | | | | Adjuvant, | |
| | | | | Fungicide, | |
| | | | | Insecticide | |
| Disodium 2,2′-oxybis(4- | 5136-51-6 | 79003 | 3175 | Microbiocide, | Soap |
| dodecylbenzene)sulfonate | | | | Adjuvant, | |
| | | | | Fungicide, | |
| | | | | Insecticide | |
| Disodium 4-dodecyl-2,4′- | 7575-62-4 | 79002 | 3172 | Microbiocide, | Soap |
| oxydibenzene sulfonate | | | | Adjuvant, | |
| | | | | Fungicide, | |
| | | | | Insecticide | |
| Dodecyl alkyl sulfonate | NA 284 | NA 553 | 1599 | Adjuvant | Soap |
| Dodecylbenzene sulfonic acid | 27176-87-0 | 98002 | 941 | Adjuvant, | Soap |
| | | | | Microbiocide, | |
| | | | | Insecticide | |
| Ethylene diamine dodecyl | 12068-06-3 | NA 257 | 3200 | Microbiocide, | Soap |
| benzene sulfonate | | | | Adjuvant, | |
| | | | | Fungicide, | |
| | | | | Insecticide | |
| Fatty acid esters | NA 1358 | NA 193 | NA 192 | Plant Growth | Soap |
| | | | | Regulator, | |
| | | | | Insecticide, | |
| | | | | Adjuvant | |
| Free fatty acids and/or amine | 84776-33-0 | 31801 | 1198 | Herbicide, Deer | Soap |
| salts | | | | Repellent, | |
| | | | | Fungicide, | |
| | | | | Insecticide | |
| Hydrogenated wax, neutral | NA 93 | NA 187 | 1368 | Adjuvant, | Soap |
| wax and alkali soaps of fatty | | | | Insecticide | |
| acids | | | | | |
| Isopropylamine | 26264-05-1 | 79030 | 3247 | Microbiocide, | Soap |
| dodecylbenzene sulfonate | | | | Adjuvant, | |
| | | | | Fungicide, | |
| | | | | Insecticide | |
| Lauryl sulfate salts | NA 937 | NA 137 | 2637 | Adjuvant, | Soap |
| | | | | Insecticide, | |
| | | | | Microbiocide, | |
| | | | | Fungicide | |
| Linear alkyl sulfonate, | NA 283 | NA 552 | 1836 | Adjuvant | Soap |
| potassium salt | | | | | |
| Magnesium lauryl sulfate | 435950 | 79017 | 1718 | Adjuvant, | Soap |
| | | | | Insecticide, | |
| | | | | Microbiocide, | |
| | | | | Fungicide | |
| Mannitan coconut oil ester | 26545-55-0 | NA 106 | 3680 | Adjuvant, | Soap |
| | | | | Soap/Surfactant | |
| Monoethanolamine dodecyl | 26545-53-9 | 79015 | 1890 | Microbiocide, | Soap |
| benzene sulfonate | | | | Adjuvant, | |
| | | | | Fungicide, | |
| | | | | Insecticide | |
| N,N-dimethyl-1,3-propane | NA 950 | NA 305 | 3168 | Microbiocide, | Soap |
| diamine dodecyl benzene | | | | Adjuvant, | |
| sulfonate | | | | Fungicide, | |
| | | | | Insecticide | |
| Naphthenic soap | 61790-13-4 | ##### | 1806 | Adjuvant, | Soap |
| | | | | Insecticide | |
| Oleic acid, methyl ester | 112-62-9 | NA 5 | 2266 | Adjuvant | Soap |
| Oleic acid, potassium salt | 143-18-0 | 79095 | 865 | Adjuvant | Soap |
| Oleic and linoleic acid, mixed | NA 4 | NA 6 | 1807 | Adjuvant | Soap |
| potassium salts | | | | | |
| Organic phosphate ester | NA 1 | NA 1 | 2725 | Soap/Surfactant, | Soap |
| surfactant | | | | Adjuvant | |
| Para-alkyl (C9-C13) benzene | 25155-30-0 | 79010 | 1739 | Microbiocide, | Soap |
| sulfonic acid, sodium salt | | | | Adjuvant, | |
| | | | | Fungicide, | |
| | | | | Insecticide | |
| Phenyl 2-trimethylammonium | 22232-15-1 | ##### | NA 125 | Microbiocide, | Soap |
| ethanesulfonate methyl sulfate | | | | Adjuvant, | |
| | | | | Fungicide, | |
| | | | | Insecticide | |
| Potash fish soap | 68154-69-8 | 79040 | 496 | Adjuvant, | Soap |
| | | | | Insecticide | |
| Potash soap | 61790-44-1 | ##### | 1596 | Herbicide, | Soap |
| | | | | Insecticide, | |
| | | | | Adjuvant | |
| Potassium alkyl benzene | 27177-77-1 | 79008 | 994 | Microbiocide, | Soap |
| sulfonates | | | | Adjuvant, | |
| | | | | Fungicide, | |
| | | | | Insecticide | |
| Potassium dodecylbenzene | 27177-77-1 | 79008 | 1027 | Microbiocide, | Soap |
| sulfonate | | | | Adjuvant, | |
| | | | | Fungicide, | |
| | | | | Insecticide | |
| Potassium laurate | 10124-65-9 | 79021 | 901 | Adjuvant, | Soap |
| | | | | Insecticide, | |
| | | | | Microbiocide, | |
| | | | | Fungicide, | |
| | | | | Soap/Surfactant | |
| Potassium laurate and myristate | NA 999 | NA 780 | 2825 | Adjuvant, | Soap |
| soaps | | | | Insecticide, | |
| | | | | Microbiocide, | |
| | | | | Fungicide | |
| Potassium laurate, other related | NA 1125 | NA 166 | 90901 | Adjuvant, | Soap |
| | | | | Insecticide, | |
| | | | | Microbiocide, | |
| | | | | Fungicide | |
| Potassium lauryl sulfate | 4706-78-9 | NA 781 | 3353 | Adjuvant | Soap |
| Potassium myristate | 13429-27-1 | 79022 | 902 | Adjuvant | Soap |
| Potassium soap of coconut | 10124-65-9 | 79021 | 903 | Adjuvant, | Soap |
| fatty acid | | | | Insecticide, | |
| | | | | Microbiocide, | |
| | | | | Fungicide, | |
| | | | | Soap/Surfactant | |
| Potassium soap of tall oil fatty | 10124-65-9 | 79021 | 1409 | Adjuvant, | Soap |
| acid | | | | Insecticide, | |
| | | | | Fungicide, | |
| | | | | Microbiocide | |
| Potassium stearate | 593-29-3 | NA 784 | 3361 | Adjuvant | Soap |
| Shampoo base | NA 460 | NA 822 | 3795 | Adjuvant | Soap |
| Soap | 68952-95-4 | 79009 | 772 | Microbiocide, | Soap |
| | | | | Insecticide, | |
| | | | | Deer Repellent | |
| Sodium alkyl* benzene | 26856-61-1 | 79087 | NA 760 | Microbiocide, | Soap |
| sulfonate *(100% C9) | | | | Adjuvant, | |
| | | | | Fungicide, | |
| | | | | Insecticide | |
| Sodium benzene sulfonate | 515-42-4 | NA 109 | 974 | Microbiocide, | Soap |
| | | | | Adjuvant, | |
| | | | | Fungicide, | |
| | | | | Insecticide | |
| Sodium decyl benzene | 1322-98-1 | 79088 | 3394 | Microbiocide, | Soap |
| sulfonate | | | | Adjuvant, | |
| | | | | Fungicide, | |
| | | | | Insecticide | |
| Sodium di (1-alkenyl) phenoxy | 39354-74-0 | 79060 | 1373 | Adjuvant | Soap |
| benzene disulfonate | | | | | |
| Sodium dodecylbenzene | 25155-30-0 | 79010 | 774 | Microbiocide, | Soap |
| sulfonate | | | | Adjuvant, | |
| | | | | Fungicide, | |
| | | | | Insecticide | |
| Sodium laurate | 629-25-4 | 79026 | NA 732 | Adjuvant, | Soap |
| | | | | Insecticide, | |
| | | | | Microbiocide, | |
| | | | | Fungicide | |
| Sodium lauryl ether diglycol | NA 1471 | NA 219 | NA 218 | Adjuvant | Soap |
| sulfate | | | | | |
| Sodium lauryl ether sulfate | 9004-82-4 | 79054 | 3799 | Adjuvant, | Soap |
| | | | | Insecticide, | |
| | | | | Microbiocide, | |
| | | | | Fungicide, | |
| | | | | Soap/Surfactant | |
| Sodium lauryl sulfate | 151-21-3 | 79011 | 907 | Adjuvant, | Soap |
| | | | | Insecticide, | |
| | | | | Microbiocide, | |
| | | | | Fungicide | |
| Sodium methylundecyl | 27987-00-4 | 79053 | NA 742 | Microbiocide, | Soap |
| benzenesulfonate | | | | Adjuvant, | |
| | | | | Fungicide, | |
| | | | | Insecticide | |
| Sodium mono (1-alkenyl) | 39354-74-0 | 79060 | 1372 | Adjuvant | Soap |
| phenoxy benzene disulfonate | | | | | |
| Sodium n-decyl sulfate | 142-87-0 | 79077 | NA 754 | Adjuvant | Soap |
| Sodium N-methyl-N-oleyl | NA 1004 | NA 854 | 3419 | Adjuvant, | Soap |
| laurate | | | | Insecticide, | |
| | | | | Microbiocide, | |
| | | | | Fungicide | |
| Sodium nonanoyloxybenzene | 91129-43-8 | 89053 | 5137 | Microbiocide, | Soap |
| sulfonate | | | | Adjuvant, | |
| | | | | Fungicide, | |
| | | | | Insecticide | |
| Sodium octylbenzene sulfonate | 28675-11-8 | 79057 | NA 745 | Microbiocide, | Soap |
| | | | | Adjuvant, | |
| | | | | Fungicide, | |
| | | | | Insecticide | |
| Sodium tallow soap | NA 473 | NA 870 | 3802 | Adjuvant, | Soap |
| | | | | Insecticide | |
| Sodium tetradecyl sulfate | 1191-50-0 | NA 110 | 3446 | Adjuvant | Soap |
| Sodium tridecylbenzene | 26248-24-8 | 79072 | NA 752 | Microbiocide, | Soap |
| sulfonate | | | | Adjuvant, | |
| | | | | Fungicide, | |
| | | | | Insecticide | |
| Sodium triisopropyl | 1323-19-9 | NA 872 | 3448 | Insecticide, | Soap |
| naphthalene sulfonate | | | | Fungicide, | |
| | | | | Microbiocide, | |
| | | | | Adjuvant | |
| Sodium triphenyl benzene | NA 1006 | NA 873 | 3449 | Microbiocide, | Soap |
| sulfonate | | | | Adjuvant, | |
| | | | | Fungicide, | |
| | | | | Insecticide | |
| Sodium xylene sulfonate | 1300-72-7 | 79019 | 909 | Adjuvant | Soap |
| Sodium-1-octane sulfonate | 5324-84-5 | NA 859 | 3886 | Adjuvant | Soap |
| Soybean fatty acids, tert amine | NA 480 | NA 884 | 1308 | Adjuvant | Soap |
| salt | | | | | |
| Strontium dodecyl benzene | 12068-15-4 | NA 906 | 3456 | Microbiocide, | Soap |
| sulfonate | | | | Adjuvant, | |
| | | | | Fungicide, | |
| | | | | Insecticide | |
| Sulfonated petroleum soap | 61789-62-6 | 79012 | NA 729 | Adjuvant, | Soap |
| | | | | Insecticide | |
| Sulfuric acid, monododecyl | 151-21-3 | 79011 | 2901 | Adjuvant, | Soap |
| ester sodium salt | | | | Insecticide, | |
| | | | | Microbiocide, | |
| | | | | Fungicide | |
| Surfactant blend | NA 1515 | NA 223 | NA 222 | Soap/Surfactant, | Soap |
| | | | | Adjuvant | |
| Surfactant mixture | NA 1516 | NA 223 | NA 223 | Soap/Surfactant, | Soap |
| | | | | Adjuvant | |
| Tallow soap | NA 607 | NA 110 | 1499 | Adjuvant, | Soap |
| | | | | Insecticide | |
| Triethanolamine | 27323-41-7 | 79020 | 984 | Microbiocide, | Soap |
| dodecylbenzene sulfonate | | | | Adjuvant, | |
| | | | | Fungicide, | |
| | | | | Insecticide | |
| Zinc dodecyl benzene sulfonate | 12068-16-5 | NA 112 | 3508 | Microbiocide, | Soap, Inorganic-Zinc |
| | | | | Adjuvant, | |
| | | | | Fungicide, | |
| | | | | Insecticide | |
| Cane syrup | NA 228 | NA 441 | 3598 | N/A | Carbohydrate |
| Complex carbohydrate polymer | NA 834 | NA 154 | 4027 | Adjuvant | Carbohydrate |
| derivative | | | | | |
| Corn syrup | NA 580 | NA 105 | 3628 | Bait | Carbohydrate |
| Cornstarch | NA 800 | NA 148 | 3627 | N/A | Carbohydrate |
| Dextrose | 50-99-7 | NA 360 | 3129 | N/A | Carbohydrate |
| Fructose | 7660-25-5 | NA 177 | 5742 | N/A | Carbohydrate |
| Honey | NA 734 | NA 134 | 2595 | N/A | Carbohydrate |
| Invert sugar | 8013-17-0 | NA 171 | 3672 | Bait | Carbohydrate |
| Invert syrup | NA 941 | NA 172 | 3673 | Bait | Carbohydrate |
| Lactose | 63-42-3 | NA 141 | 1400 | N/A | Carbohydrate |
| Malt extract | 8002-48-0 | NA 134 | 2659 | N/A | Carbohydrate |
| Molasses | NA 40 | NA 69 | 744 | Bait | Carbohydrate |
| Polysaccharide polymer | NA 702 | NA 129 | 2112 | Adjuvant | Carbohydrate |
| Sorbitol | 50-70-4 | NA 214 | 1629 | N/A | Carbohydrate |
| Starch | 9005-84-9 | NA 898 | 1821 | N/A | Carbohydrate |
| Starch-G-poly (acrylamide-co- | NA 494 | NA 899 | 2888 | N/A | Carbohydrate |
| sodium acrylate) | | | | | |
| Sugar | 57-50-1 | 23 | 708 | Bait | Carbohydrate |
| Sweet n′ neat 45 spray dried | NA 634 | NA 116 | 3565 | N/A | Carbohydrate |
| honey powder | | | | | |
| Validamycin | 37248-47-8 | NA 190 | NA 189 | Fungicide | Carbohydrate |
| alpha-Cellulose | NA 1537 | NA 225 | NA 224 | Insecticide | Cellulose derivative |
| Carbo methoxy ether cellulose, | 9004-32-4 | ##### | 2446 | Insecticide | Cellulose derivative |
| sodium salt | | | | | |
| Cellulose | 9004-34-6 | NA 432 | 3100 | N/A | Cellulose derivative |
| Cellulose ethyl hydroxy ethyl | 9004-58-4 | NA 113 | 3101 | N/A | Cellulose derivative |
| ether | | | | | |
| Ethyl cellulose | 9004-57-3 | NA 264 | 3652 | N/A | Cellulose derivative |
| Hydroxyethyl cellulose | 9004-62-0 | 46201 | 1511 | N/A | Cellulose derivative |
| Hydroxypropyl methyl | 9004-65-3 | NA 179 | 1969 | N/A | Cellulose derivative |
| cellulose | | | | | |
| Methyl cellulose | 9004-67-5 | NA 85 | 1205 | N/A | Cellulose derivative |
| Microcrystalline cellulose | 9004-34-6 | NA 63 | 2686 | N/A | Cellulose derivative |
| Nitrocellulose | 9004-70-0 | 99601 | NA 867 | N/A | Cellulose derivative |
| (2- | 41289-08-1 | ##### | NA 101 | N/A | Unclassified |
| chloroethyl)methylbis(phenylmethoxy) | | | | | |
| silane | | | | | |
| 1% verde green solution | NA 553 | NA 100 | 2982 | Dye | Unclassified |
| 1,2-dimethyl hydrazine | 540-73-0 | NA 142 | 3165 | N/A | Unclassified |
| 1,2-epoxy butane | 106-88-7 | NA 142 | 3190 | N/A | Unclassified |
| 1,3-butadiene | 106-99-0 | NA 471 | 2431 | N/A | Unclassified |
| 1-((4-methyl-2-nitrophenyl) | NA 43 | NA 80 | 2682 | N/A | Unclassified |
| azo) naphthalenol | | | | | |
| 1-methyl pyrrolidine | 120-94-5 | NA 75 | 3287 | N/A | Unclassified |
| 1-Pentanethiol | 110-66-7 | ##### | NA 872 | N/A | Unclassified |
| 2,2′-octadecyl imino diethanol | 10213-78-2 | NA 20 | 3306 | N/A | Unclassified |
| 2,2′-oxybis (4,4,6-trimethyl- | 14697-50-8 | 12402 | 792 | Microbiocide | Unclassified |
| 1,3,2-dioxaborinane) | | | | | |
| 2-butanone oxime | 96-29-7 | NA 474 | 3071 | N/A | Unclassified |
| 2-chloropyridine-N-oxide | NA 219 | NA 417 | 2466 | N/A | Unclassified |
| 2-ethyl hexanoic acid | 149-57-5 | ##### | 3228 | N/A | Unclassified |
| 2-ethyl hexyl acrylate | 103-11-7 | NA 252 | 2115 | N/A | Unclassified |
| 2-heptanone | 110-43-0 | NA 206 | 3223 | N/A | Unclassified |
| 2-hydroxy-4-n-octyl | NA 572 | NA 103 | 2608 | N/A | Unclassified |
| benzophenone | | | | | |
| 2-mercapto pyridine | 2637-34-5 | NA 94 | 2671 | N/A | Unclassified |
| 2-nitro-1-butyl phosphate | 57249-19-1 | 56902 | 2033 | N/A | Unclassified |
| 2-Nitropropane | 79-46-9 | ##### | 3301 | Impurity | Unclassified |
| 3′,6′-Dihydro-2′,4′,5′,7′- | 15905-32-5 | ##### | NA 981 | Dye | Unclassified |
| tetraiodospiro(isobenzofuran- | | | | | |
| 1(3H),9′-(9H)xanthen)-3-one | | | | | |
| 4-hydroxydiphenylamine | 122-37-2 | NA 217 | NA 217 | Breakdown | Unclassified |
| | | | | product | |
| 5-methyl-2-hexanone | 110-12-3 | NA 81 | 3230 | N/A | Unclassified |
| Acetaldehyde | 75-07-0 | ##### | 3012 | N/A | Unclassified |
| Acetic anhydride | 108-24-7 | 44007 | 3014 | N/A | Unclassified |
| Acetone | 67-64-1 | 44101 | 747 | Solvent | Unclassified |
| Acetonitrile | 75-05-8 | NA 699 | 3015 | Solvent | Unclassified |
| Acetophenone | 98-86-2 | ##### | 2074 | N/A | Unclassified |
| Acid blue 182 | NA 599 | NA 108 | 2353 | Dye | Unclassified |
| Acid blue 7 | NA 385 | NA 702 | 2352 | Dye | Unclassified |
| Acid blue 9, diammonium salt | 2650-18-2 | ##### | 2154 | Herbicide, Dye | Unclassified |
| Acid blue 9, disodium salt | 3844-45-9 | ##### | 3562 | Herbicide, Dye | Unclassified |
| Acid yellow 23 | 1934-21-0 | ##### | 2155 | Herbicide, Dye | Unclassified |
| Acridinic bases | NA 1356 | NA 193 | NA 192 | Repellent | Unclassified |
| Acrylic acid | 79-10-7 | NA 129 | 2118 | N/A | Unclassified |
| AD-67 | NA 1378 | NA 195 | NA 194 | Herbicide | Unclassified |
| | | | | Safener | |
| Adjuvants | NA 1484 | NA 221 | NA 220 | Adjuvant | Unclassified |
| Agent 1059-106 | NA 388 | NA 708 | 2357 | N/A | Unclassified |
| Agent 1059-108 | NA 600 | NA 108 | 2358 | N/A | Unclassified |
| Agent 1315-33A | NA 389 | NA 709 | 3881 | N/A | Unclassified |
| Agent 1315-33N | NA 390 | NA 710 | 3879 | N/A | Unclassified |
| Agent 1594-9 | NA 654 | NA 120 | 3880 | N/A | Unclassified |
| Agent 551-85 | NA 387 | NA 707 | 2356 | N/A | Unclassified |
| Agent W-218 | NA 391 | NA 711 | 2359 | N/A | Unclassified |
| Aldehyde C10 | 112-31-2 | ##### | NA 107 | N/A | Unclassified |
| Aldehyde C11 | 112-44-7 | ##### | NA 107 | N/A | Unclassified |
| Aldehyde C12 | 112-54-9 | ##### | NA 107 | N/A | Unclassified |
| Aldehyde C6 | 66-25-1 | ##### | NA 107 | N/A | Unclassified |
| Aldehyde C8 | 124-13-0 | ##### | NA 107 | N/A | Unclassified |
| Aldimorph | NA 1340 | NA 188 | NA 187 | Fungicide | Unclassified |
| Aliphatic amines | NA 713 | NA 130 | 2362 | N/A | Unclassified |
| Alkanol XC | NA 828 | NA 152 | 3931 | Adjuvant | Unclassified |
| Alkatronic PGP-33-4 | NA 395 | NA 717 | 2363 | N/A | Unclassified |
| Alkyl (C9H12) benzenes | NA 310 | NA 589 | 2371 | Solvent | Unclassified |
| Amine 220 | 21652-27-7 | NA 141 | 3033 | N/A | Unclassified |
| Amines, aliphatic | NA 277 | NA 543 | 1747 | N/A | Unclassified |
| Amino acids | NA 1359 | NA 193 | NA 192 | Plant Growth | Unclassified |
| | | | | Regulator | |
| Ammonium propionate | NA 1061 | NA 158 | 5015 | Microbiocide, | Unclassified |
| | | | | Preservative | |
| Amsco-solv | NA 271 | NA 526 | 2386 | Solvent | Unclassified |
| Amygdalinic acid diethyl | NA 1448 | NA 215 | NA 214 | Insect | Unclassified |
| amide | | | | Repellent | |
| Amyl acetate | 628-63-7 | 169 | 1889 | N/A | Unclassified |
| Aniline | 62-53-3 | ##### | 3056 | N/A | Unclassified |
| Anionic wax emulsion | 61789-97-7 | NA 515 | 3818 | N/A | Unclassified |
| Anionic wax emulsion | NA 892 | NA 171 | 3848 | N/A | Unclassified |
| Aquashade | 92170-50-8 | ##### | NA 909 | Herbicide, Dye | Unclassified |
| Armul 1426 | NA 267 | NA 522 | 3878 | N/A | Unclassified |
| Armul 1426 HF | NA 822 | NA 151 | 3877 | N/A | Unclassified |
| Armul 1427 | NA 268 | NA 523 | 3883 | N/A | Unclassified |
| Armul 1427 HF | NA 589 | NA 106 | 3876 | N/A | Unclassified |
| Armul 214/215 | NA 265 | NA 520 | 2391 | N/A | Unclassified |
| Armul 22 | NA 263 | NA 518 | 2389 | N/A | Unclassified |
| Armul 33 | NA 587 | NA 106 | 2390 | N/A | Unclassified |
| Armul 358 | NA 266 | NA 521 | 2392 | N/A | Unclassified |
| Armul 646 | NA 588 | NA 106 | 2393 | N/A | Unclassified |
| Armul 88 | NA 264 | NA 519 | 3908 | N/A | Unclassified |
| Artificial peanut butter flavor | NA 717 | NA 131 | 2397 | N/A | Unclassified |
| wl-5344 | | | | | |
| Astrazon yellow 4g200 | NA 258 | NA 511 | 2399 | Dye | Unclassified |
| Atlox 1045A | 61723-83-9 | NA 152 | 3911 | N/A | Unclassified |
| Atplus 300F | NA 255 | NA 507 | 3916 | N/A | Unclassified |
| b-Bromo-b-nitrostyrene | 7166-19-0 | NA 221 | NA 221 | Microbiocide | Unclassified |
| Barochem B464 | NA 247 | NA 499 | 2413 | N/A | Unclassified |
| Basic violet 1 | 8004-87-3 | 39503 | 3584 | Dye | Unclassified |
| BCP | NA 237 | NA 465 | 64 | N/A | Unclassified |
| Be-4526 phthalo blue lake r-c | NA 244 | NA 488 | 3518 | Dye | Unclassified |
| Benfuresate | 68505-69-1 | NA 187 | NA 187 | Herbicide | Unclassified |
| Bensultap | 17606-31-4 | NA 185 | NA 185 | Insecticide | Unclassified |
| Benzene sulfonyl chloride | 98-09-9 | NA 489 | 2415 | N/A | Unclassified |
| Benzfendizone | 158755-95-4 | NA 230 | NA 230 | Herbicide | Unclassified |
| Benzobicyclon | 156963-66-5 | NA 230 | NA 230 | Herbicide | Unclassified |
| Benzofenap | 82692-44-2 | NA 211 | NA 211 | Herbicide | Unclassified |
| Benzophenone-2 | 131-55-5 | NA 492 | 3063 | N/A | Unclassified |
| Benzophenone-3 | 119-61-9 | 315 | 2417 | N/A | Unclassified |
| Benzyl chloride | 100-44-7 | NA 486 | 2419 | N/A | Unclassified |
| Berol 927 | NA 894 | NA 171 | 3874 | N/A | Unclassified |
| Berol 968 | NA 893 | NA 171 | 3873 | N/A | Unclassified |
| Bethoxazin | 163269-30-5 | NA 230 | NA 230 | Herbicide | Unclassified |
| BHA | 25013-16-5 | NA 484 | 2420 | N/A | Unclassified |
| Bicyclic and methene resins | NA 242 | NA 485 | 3516 | N/A | Unclassified |
| Bioban p-1487 | 37304-88-4 | ##### | 1742 | Microbiocide | Unclassified |
| Biphenyl | 92-52-4 | 17002 | 227 | Microbiocide, | Unclassified |
| | | | | Fungicide | |
| Bisphenol A | 80-05-7 | NA 482 | 3070 | N/A | Unclassified |
| Bisphenol F | 205672 | NA 216 | NA 215 | N/A | Unclassified |
| Bispyribac | 125401-75-4 | NA 203 | NA 203 | Herbicide | Unclassified |
| Bisthiosemi | 39603-48-0 | NA 206 | NA 206 | Rodenticide | Unclassified |
| Block polymer of | NA 850 | NA 158 | 5011 | N/A | Unclassified |
| carbonyldiamide | | | | | |
| polyoxyalkylated glycol adduct | | | | | |
| Brandol | NA 1379 | NA 195 | NA 194 | Fungicide | Unclassified |
| Bromoacetic acid | 79-08-3 | 8702 | NA 102 | N/A | Unclassified |
| Burgundy mixture | NA 1380 | NA 195 | NA 194 | Fungicide | Unclassified |
| Busan 1009 | NA 1564 | NA 227 | NA 227 | Fungicide | Unclassified |
| Butadiene-acrylonitrile | 9003-18-3 | NA 472 | 2432 | N/A | Unclassified |
| copolymer | | | | | |
| Butane | 106-97-8 | NA 473 | 1562 | N/A | Unclassified |
| Buthiobate | 51308-54-4 | NA 206 | NA 206 | Fungicide | Unclassified |
| Butyl mercaptan | 109-79-5 | ##### | 5087 | Deer Repellent, | Unclassified |
| | | | | Bear Repellent | |
| C-PA-1224 black | NA 191 | NA 379 | 2483 | N/A | Unclassified |
| Calcium propionate | 4075-81-4 | 77701 | 2441 | Preservative | Unclassified |
| Cambendichlor | 56141-00-5 | NA 230 | NA 230 | Herbicide | Unclassified |
| Capric acid | 334-48-5 | ##### | 2315 | Microbiocide | Unclassified |
| Caprylic acid | 124-07-2 | ##### | 2316 | Microbiocide | Unclassified |
| Caramel color | NA 720 | NA 131 | 2444 | Dye | Unclassified |
| Carfentrazone | 128621-72-7 | NA 210 | NA 210 | Herbicide | Unclassified |
| Carfentrazone-ethyl | 128639-02-1 | ##### | 5130 | Herbicide | Unclassified |
| Carpropamid | 104030-54-8 | NA 203 | NA 203 | Fungicide | Unclassified |
| Cartap | 15263-53-3 | NA 181 | NA 181 | Insecticide | Unclassified |
| Cartap hydrochloride | 22042-59-7 | NA 182 | NA 181 | Insecticide | Unclassified |
| (unspecified hydrochloride) | | | | | |
| Cartap monohydrochloride | 15263-52-2 | NA 182 | NA 181 | Insecticide | Unclassified |
| Cetiol HE | 66105-29-1 | ##### | NA 871 | Insecticide | Unclassified |
| CGA-154281 | NA 721 | NA 132 | 2451 | N/A | Unclassified |
| Chartersol-1 | NA 221 | NA 428 | 2453 | Solvent | Unclassified |
| Chem-o-thane D-3860/D-3861 | NA 223 | NA 430 | 2454 | N/A | Unclassified |
| Chicago sludge | 68188-15-8 | NA 147 | 3605 | N/A | Unclassified |
| Chloracetic acid | 79-11-8 | ##### | 3102 | N/A | Unclassified |
| Chloral-bis-acylal | NA 1344 | NA 191 | NA 191 | Plant Growth | Unclassified |
| | | | | Regulator | |
| Chloral-semi-acetal | NA 1345 | NA 191 | NA 191 | Herbicide | Unclassified |
| Chloretazate | NA 1403 | NA 197 | NA 196 | Plant Growth | Unclassified |
| | | | | Regulator | |
| Chlorfenprop | 59404-06-7 | NA 181 | NA 180 | Herbicide | Unclassified |
| Chlorfenprop-methyl | 14437-17-3 | ##### | NA 156 | Herbicide | Unclassified |
| Chlorinated rubber | 2594016 | NA 427 | 3606 | N/A | Unclassified |
| Chlormethiuron | 28217-97-2 | NA 206 | NA 206 | Insecticide | Unclassified |
| Chlorobenzene | 108-90-7 | 56504 | 2460 | N/A | Unclassified |
| Choline chloride | 67-48-1 | NA 191 | NA 190 | Rodenticide | Unclassified |
| Cinidon-ethyl | 142891-20-1 | NA 216 | NA 215 | Herbicide | Unclassified |
| Citowett | NA 218 | NA 412 | 2471 | Soap/Surfactant | Unclassified |
| Cloquintocet | 88349-88-6 | NA 219 | NA 218 | Herbicide | Unclassified |
| | | | | Safener | |
| Cloquintocet mexyl | 99607-70-2 | NA 191 | NA 190 | Herbicide | Unclassified |
| | | | | Safener | |
| Cloxyfonac | 32791-87-0 | NA 204 | NA 203 | Plant Growth | Unclassified |
| | | | | Regulator | |
| Colgate crystal white hand | NA 202 | NA 393 | 2476 | N/A | Unclassified |
| dishwashing liquid | | | | | |
| Collagen | NA 203 | NA 394 | 1805 | N/A | Unclassified |
| Colloid 643 | NA 582 | NA 105 | 2477 | N/A | Unclassified |
| Colloid 840 | NA 791 | NA 146 | 3527 | N/A | Unclassified |
| Cookies | NA 641 | NA 117 | 3621 | Bait | Unclassified |
| Credazine | 14491-59-9 | NA 207 | NA 206 | Herbicide | Unclassified |
| Cubiet | NA 1367 | NA 193 | NA 193 | Fungicide, | Unclassified |
| | | | | Pruning Aid | |
| Cyclohexanone | 108-94-1 | 25902 | 177 | Solvent | Unclassified |
| Cyclopentadiene polymer | NA 1450 | NA 215 | NA 214 | Insecticide | Unclassified |
| Cymiazol hydrochloride | 61676-87-7 | NA 215 | NA 214 | Insecticide | Unclassified |
| Cymoxanil | 57966-95-7 | ##### | 4002 | Fungicide | Unclassified |
| Cyometrinil | 63278-33-1 | NA 207 | NA 206 | Herbicide | Unclassified |
| | | | | Safener | |
| Cysteine | 52-90-4 | NA 190 | NA 190 | Plant Growth | Unclassified |
| | | | | Regulator | |
| D & C red no. 28 | 18472-87-2 | ##### | 3515 | Dye | Unclassified |
| D & C red no. 37 | 1632455 | NA 117 | 3634 | Dye | Unclassified |
| D & C yellow 10 lake | 68814-04-0 | NA 365 | 2487 | Dye | Unclassified |
| D & C yellow no. 8 | NA 829 | NA 152 | 3991 | Dye | Unclassified |
| D-425 | NA 184 | NA 367 | 2489 | N/A | Unclassified |
| D-79 solvent | NA 183 | NA 366 | 2488 | Solvent | Unclassified |
| Daxad 23 | NA 185 | NA 368 | 2491 | N/A | Unclassified |
| Day-glo saturn yellow AX-17-N | NA 186 | NA 369 | 2492 | Dye | Unclassified |
| DE-570 | NA 1459 | NA 216 | NA 216 | N/A | Unclassified |
| Delvet 65 | NA 177 | NA 357 | 2494 | N/A | Unclassified |
| Desoto urethane resin | NA 179 | NA 359 | 2497 | N/A | Unclassified |
| Device (swimming pool | NA 1503 | NA 222 | NA 222 | Algaecide, | Unclassified |
| algaecide, bacteriacide, | | | | Microbiocide, | |
| molluscicide) no guarantee | | | | Molluscicide | |
| required | | | | | |
| Device, no guarantee required | NA 1502 | NA 222 | NA 221 | N/A | Unclassified |
| Di-1-p-menthene | NA 1381 | NA 195 | NA 194 | Plant Growth | Unclassified |
| | | | | Regulator | |
| Diafenthiuron | 80060-09-9 | NA 186 | NA 185 | Insecticide | Unclassified |
| Dichloro acetic acid | 79-43-6 | NA 105 | 3133 | N/A | Unclassified |
| Dichlorovinyl acid | NA 174 | NA 347 | 2502 | Breakdown | Unclassified |
| | | | | product | |
| Diclobutrazol | 75736-33-3 | NA 186 | NA 185 | Fungicide | Unclassified |
| Diclomezine | 62865-36-5 | NA 204 | NA 204 | Fungicide | Unclassified |
| Dicyclanil | 112636-83-6 | NA 211 | NA 211 | Insect Growth | Unclassified |
| | | | | Regulator | |
| Dicyclopentadiene | 77-73-6 | NA 186 | NA 185 | Plant Growth | Unclassified |
| | | | | Regulator, Dog | |
| | | | | and Cat | |
| | | | | Repellent | |
| Dicyclopentadiene linseed oil | 68213-53-6 | 31612 | NA 291 | N/A | Unclassified |
| copolymer | | | | | |
| Diethyl phosphate | 598-02-7 | NA 180 | NA 179 | Breakdown | Unclassified |
| | | | | product | |
| Diethyl sulfide | 352-93-2 | ##### | 5088 | Bear Repellent | Unclassified |
| Diethyldithio phosphate | 298-06-6 | NA 180 | NA 179 | Breakdown | Unclassified |
| | | | | product | |
| Diethylthio phosphate | 5871-17-0 | NA 180 | NA 179 | Breakdown | Unclassified |
| | | | | product | |
| Diisoamyl ketone | 2050-99-9 | NA 325 | 3155 | Solvent | Unclassified |
| Diisobutyl ketone | 108-83-8 | 44107 | 1946 | Solvent | Unclassified |
| Dikegulac | 18467-77-1 | NA 181 | NA 180 | Herbicide | Unclassified |
| Dikegulac sodium | 52508-35-7 | ##### | 2004 | Plant Growth | Unclassified |
| | | | | Regulator | |
| Dimer acid | 6144-28-1 | NA 330 | 3158 | N/A | Unclassified |
| Dimethyl phosphate | 813-78-5 | NA 180 | NA 179 | Breakdown | Unclassified |
| | | | | product | |
| Dimethyl sulfoxide | 67-68-5 | 177 | 3169 | Solvent | Unclassified |
| Dimethyldithio phosphate | 756-80-9 | NA 180 | NA 180 | Breakdown | Unclassified |
| | | | | product | |
| Dimethylthio phosphate | NA 1339 | NA 180 | NA 179 | Breakdown | Unclassified |
| | | | | product | |
| Dioxane | 123-91-1 | NA 300 | 2529 | Solvent, | Unclassified |
| | | | | Impurity | |
| Diphenyl methane | 101-81-5 | NA 301 | 3171 | N/A | Unclassified |
| Diphenylamine | 122-39-4 | 38501 | 228 | Fungicide, | Unclassified |
| | | | | Plant Growth | |
| | | | | Regulator, | |
| | | | | Insecticide | |
| Dipropyl isocinchomerate | 3737-22-2 | NA 204 | NA 204 | Insect | Unclassified |
| | | | | Repellent | |
| Dipropyl isocinchomeronate | 136-45-8 | 47201 | 681 | Insect | Unclassified |
| | | | | Repellent | |
| Dog or cat collars | NA 155 | NA 291 | 3645 | N/A | Unclassified |
| Dow Corning FG-10 | NA 147 | NA 281 | 2541 | N/A | Unclassified |
| Dowanol desg solvent | NA 725 | NA 133 | 2538 | Solvent | Unclassified |
| Drewplus L-768 | NA 148 | NA 283 | 2542 | N/A | Unclassified |
| Dyestuffs | NA 689 | NA 127 | 1913 | Dye | Unclassified |
| Emcol AL69-49 | NA 726 | NA 133 | 2544 | N/A | Unclassified |
| Emphos CS-121 | NA 727 | NA 133 | 2545 | Soap/Surfactant | Unclassified |
| Emulgator | NA 143 | NA 277 | 2546 | N/A | Unclassified |
| Emulsogen CP 136 | NA 576 | NA 104 | 2547 | Adjuvant | Unclassified |
| Emulsogen NP 11-300 | NA 144 | NA 278 | 2548 | Adjuvant | Unclassified |
| Endothall | 145-73-3 | 38901 | 5813 | Herbicide, | Unclassified |
| | | | | Defoliant | |
| Endothall, di (N,N- | NA 949 | NA 271 | 1355 | Herbicide, | Unclassified |
| diethylalkylamine) salt | | | | Defoliant | |
| Endothall, di (N,N- | 66330-88-9 | 38905 | 1904 | Herbicide, | Unclassified |
| dimethylalkylamine) salt | | | | Defoliant | |
| Endothall, di(N,N- | 2536-26-7 | 38902 | NA 323 | Herbicide, | Unclassified |
| dimethyltridecylamine) salt | | | | Defoliant | |
| Endothall, diammonium salt | 17439-94-0 | 38908 | NA 326 | Herbicide, | Unclassified |
| | | | | Defoliant | |
| Endothall, dipotassium salt | 2164-07-0 | 38904 | 1356 | Herbicide, | Unclassified |
| | | | | Defoliant | |
| Endothall, disodium salt | 129-67-9 | 38903 | 260 | Herbicide, | Unclassified |
| | | | | Defoliant | |
| Endothall, mono (N,N-diethyl | NA 926 | NA 4 | 1354 | Herbicide, | Unclassified |
| alkylamine) salt | | | | Defoliant | |
| Endothall, mono [N,N- | NA 929 | NA 61 | 2056 | Herbicide, | Unclassified |
| dimethyl alkylamine] salt | | | | Defoliant | |
| Endothall, mono(N,N- | 2536-27-8 | 38906 | NA 325 | Herbicide, | Unclassified |
| dimethyltridecylamine) salt | | | | Defoliant | |
| Endothall, N,N- | NA 1282 | 38907 | NA 174 | Herbicide, | Unclassified |
| dimethylcocamine salt (1:2) | | | | Algaecide | |
| Epoxidized linseed oil | 2232670 | NA 272 | 2550 | Insecticide, | Unclassified |
| | | | | Adjuvant | |
| Epoxy resins | NA 139 | NA 269 | 3650 | N/A | Unclassified |
| Erythrosine B | 16423-68-0 | ##### | 2224 | Dye, | Unclassified |
| | | | | Insecticide | |
| Escorez 1102 | 9048-47-9 | NA 146 | 3546 | N/A | Unclassified |
| Espesol I | NA 140 | NA 270 | 2552 | Solvent | Unclassified |
| Etacelasil | 37894-46-5 | NA 188 | NA 187 | Defoliant, Plant | Unclassified |
| | | | | Growth | |
| | | | | Regulator | |
| Ethanethiol | 75-08-1 | NA 187 | NA 186 | Rodenticide | Unclassified |
| Ethomeen | NA 136 | NA 265 | 2553 | N/A | Unclassified |
| Ethyl acrylate | 140-88-5 | NA 259 | 2117 | N/A | Unclassified |
| Ethyl formate | 109-94-4 | 43102 | 278 | Fumigant, | Unclassified |
| | | | | Insecticide | |
| Ethyl methacrylate | 97-63-2 | NA 248 | 3203 | N/A | Unclassified |
| Ethylene-acrylic acid | 9010-77-9 | NA 253 | 3653 | Adjuvant | Unclassified |
| copolymer | | | | | |
| Ethylene-acrylic terpolymer | NA 133 | NA 254 | 1950 | Adjuvant | Unclassified |
| Ethylhexanoate | 123-66-0 | NA 190 | NA 190 | Fungicide, | Unclassified |
| | | | | Microbiocide | |
| Extender | NA 1377 | NA 195 | NA 194 | Synergist | Unclassified |
| Fatty alcohols | NA 1364 | NA 193 | NA 192 | Plant Growth | Unclassified |
| | | | | Regulator, | |
| | | | | Adjuvant | |
| FD&C blue no. 1 aluminum | 53026-57-6 | NA 242 | 3000 | Dye | Unclassified |
| lake | | | | | |
| FD&C green no. 3 | NA 127 | NA 243 | 2566 | Dye | Unclassified |
| FD&C red no. 40 | NA 947 | ##### | 2567 | Dye, Impurity | Unclassified |
| Feed supplements | NA 128 | NA 244 | 3655 | N/A | Unclassified |
| Fenchlorazole | 103112-35-2 | NA 181 | NA 181 | Herbicide | Unclassified |
| | | | | Safener | |
| Fenchlorim | 3740-92-9 | NA 182 | NA 181 | Herbicide | Unclassified |
| | | | | Safener | |
| Fenitropan | 65934-95-4 | NA 207 | NA 207 | Fungicide | Unclassified |
| Fenpiclonil | 74738-17-3 | NA 188 | NA 187 | Fungicide | Unclassified |
| Fenpropidin | 67306-00-7 | NA 184 | NA 183 | Fungicide | Unclassified |
| Fenpropimorph | 67306-03-0 | ##### | NA 988 | Fungicide | Unclassified |
| Fenpropimorph | 67564-91-4 | NA 201 | NA 200 | Fungicide | Unclassified |
| Fenthiosulf | NA 1406 | NA 198 | NA 197 | Insecticide | Unclassified |
| Fertilizer, general | NA 729 | NA 133 | 2569 | N/A | Unclassified |
| Filter paper e-d-601-25 | NA 122 | NA 235 | 3525 | N/A | Unclassified |
| Flomo 1212 | NA 124 | NA 239 | 2572 | Soap/Surfactant | Unclassified |
| Flomo 4NP | NA 616 | NA 112 | 2573 | Soap/Surfactant | Unclassified |
| Florasulam | NA 1447 | NA 214 | NA 214 | Herbicide | Unclassified |
| Flubenzimine (unstated | 37893-02-0 | NA 188 | NA 187 | Insecticide | Unclassified |
| stereochemistry) | | | | | |
| Flumequine | 42835-25-6 | NA 188 | NA 187 | Microbiocide | Unclassified |
| Flumiclorac | 87547-04-4 | NA 232 | NA 232 | Herbicide | Unclassified |
| Flumipropyn | 84478-52-4 | NA 229 | NA 229 | Herbicide | Unclassified |
| Flupropanate | 756-09-2 | NA 204 | NA 204 | Herbicide | Unclassified |
| Flupropanate, sodium salt | 22898-01-7 | NA 211 | NA 211 | Herbicide | Unclassified |
| Flurazole | 72850-64-7 | NA 187 | NA 186 | Herbicide | Unclassified |
| | | | | Safener | |
| Flurtamone | 96525-23-4 | NA 217 | NA 216 | Herbicide | Unclassified |
| Fluthiacet | 149253-65-6 | NA 205 | NA 204 | Herbicide | Unclassified |
| Fluxofenim | 88485-37-4 | NA 202 | NA 201 | Herbicide | Unclassified |
| | | | | Safener | |
| Formaldehyde | 50-00-0 | 43001 | 295 | Microbiocide, | Unclassified |
| | | | | Fungicide | |
| Fosetyl-A1 | 39148-24-8 | ##### | 2210 | Fungicide | Unclassified |
| Fragrance 1256 H | NA 115 | NA 227 | 2575 | Fragrance | Unclassified |
| Fragrance for mosquito | 51843 | NA 140 | 3007 | Fragrance | Unclassified |
| repellent p-7510 | | | | | |
| Fragrance N-5716 | NA 116 | NA 228 | 3852 | Fragrance | Unclassified |
| Fragrance orange 418228 | NA 117 | NA 229 | 3889 | Fragrance | Unclassified |
| Fragrance PNLF 6986 | NA 633 | NA 116 | 3563 | Fragrance | Unclassified |
| Fragrance RS-2661 | NA 824 | NA 151 | 3891 | Fragrance | Unclassified |
| Frambinone | 5471-51-2 | NA 212 | NA 212 | Fragrance | Unclassified |
| Fumaric acid | 110-17-8 | 51201 | 297 | N/A | Unclassified |
| Fumaric and crotonic acids, | NA 118 | NA 230 | 1711 | N/A | Unclassified |
| esters of | | | | | |
| Fumigants | NA 1492 | NA 221 | NA 221 | Fumigant | Unclassified |
| Furaneol 15% p.g. | NA 119 | NA 231 | 2576 | N/A | Unclassified |
| Furfural | 98-01-1 | 43301 | 1522 | N/A | Unclassified |
| Gentian | 97676-22-7 | ##### | NA 108 | Dye | Unclassified |
| Gentian violet | 548-62-9 | 39502 | 1270 | Microbiocide, | Unclassified |
| | | | | Dye | |
| Gentian violet | 548-62-9 | 98401 | NA 173 | Microbiocide, | Unclassified |
| | | | | Dye | |
| Givaudan muquet PA-6664 | NA 575 | NA 104 | 2580 | N/A | Unclassified |
| Glidden no. 3200 white latex | NA 113 | NA 224 | 2581 | N/A | Unclassified |
| Glue | NA 643 | NA 118 | 3661 | N/A | Unclassified |
| Glufosinate | 51276-47-2 | NA 203 | NA 202 | Herbicide | Unclassified |
| Glufosinate-ammonium | 77182-82-2 | ##### | 3946 | Herbicide | Unclassified |
| Grease (bands, fruit frees) | NA 1350 | NA 192 | NA 191 | Insecticide | Unclassified |
| Green M liquid dye | NA 106 | NA 211 | 3840 | Dye | Unclassified |
| Heptopargil | 73886-28-9 | NA 208 | NA 207 | Plant Growth | Unclassified |
| | | | | Regulator | |
| Heptyl butyrate | 5870-93-9 | NA 207 | 3668 | N/A | Unclassified |
| Hi-sol 15 | NA 97 | NA 192 | 2594 | Solvent | Unclassified |
| Hostaphat MDAR-N-040 | NA 98 | NA 193 | 2596 | N/A | Unclassified |
| Hostapon T powder highly | 97-80-3 | NA 171 | 3942 | N/A | Unclassified |
| concentrated | | | | | |
| Hydrazine | 302-01-2 | NA 195 | 2598 | Impurity, | Unclassified |
| | | | | Breakdown | |
| | | | | product | |
| Hydrolysed linseed fatty acid | NA 1436 | NA 212 | NA 212 | N/A | Unclassified |
| Hydroxyl amine sulfate | 10039-54-0 | NA 181 | 3235 | N/A | Unclassified |
| Hydroxyphenyl-salicylamide | NA 1396 | NA 197 | NA 196 | Fungicide | Unclassified |
| Inabenfide | 82211-24-3 | NA 205 | NA 204 | Plant Growth | Unclassified |
| | | | | Regulator | |
| Indanofan | 133220-30-1 | NA 232 | NA 232 | Herbicide | Unclassified |
| Indigo carmine | 860-22-0 | NA 146 | 3530 | Dye | Unclassified |
| Inert (VOC reevaluation) | NA 877 | NA 169 | 3973 | N/A | Unclassified |
| Inert ingredients | NA 872 | NA 160 | 0 | N/A | Unclassified |
| Instex 10100 color concentrate | NA 88 | NA 170 | 2615 | Dye | Unclassified |
| (black) | | | | | |
| Irganox 1010 | 6683-19-8 | NA 173 | 3623 | N/A | Unclassified |
| Irganox 1010 | 6683-19-8 | NA 170 | 3523 | N/A | Unclassified |
| Irganox 1076 | 2082-79-3 | NA 174 | 2617 | N/A | Unclassified |
| Iso-amyl mercaptan | 541-31-1 | NA 213 | NA 212 | N/A | Unclassified |
| Isobutyl acetate | 110-19-0 | NA 143 | 3241 | N/A | Unclassified |
| Isobutyric acid | 79-31-2 | ##### | 2110 | Preservative | Unclassified |
| Isooctyl phosphate | 12645-53-3 | NA 162 | 3258 | Soap/Surfactant, | Unclassified |
| | | | | Adjuvant | |
| Isopropanolamine nitrite | NA 87 | NA 166 | 3245 | N/A | Unclassified |
| Isopropylamine | 75-31-0 | NA 157 | 3246 | N/A | Unclassified |
| Isoprothiolane | 50512-35-1 | NA 189 | NA 188 | Fungicide | Unclassified |
| Isoval | NA 1385 | NA 195 | NA 195 | Rodenticide | Unclassified |
| Isoxapyrifop | 87757-18-4 | NA 208 | NA 208 | Herbicide | Unclassified |
| Ivory snow | NA 75 | NA 146 | 3674 | N/A | Unclassified |
| K 1A112 | NA 77 | NA 148 | 2628 | N/A | Unclassified |
| Kelsol 5134 | 68309-52-4 | NA 149 | 3526 | Solvent | Unclassified |
| Ketoconazole | 65277-42-1 | NA 216 | NA 215 | Fungicide | Unclassified |
| Korax | 2425-66-3 | 11302 | 351 | N/A | Unclassified |
| Krynac 1122 | NA 79 | NA 151 | 2632 | N/A | Unclassified |
| Latex paint | NA 74 | NA 144 | 2634 | N/A | Unclassified |
| Leonil DB powder | NA 71 | NA 139 | 2639 | N/A | Unclassified |
| Lignoflex | NA 68 | NA 131 | 3265 | N/A | Unclassified |
| Limonia 19-11189 | NA 69 | NA 132 | 2643 | N/A | Unclassified |
| Linseed-soya alkyd resin | NA 62 | NA 117 | 2646 | N/A | Unclassified |
| Lithium | 29457-72-5 | 75004 | 5678 | Insecticide, | Unclassified |
| (perfluorooctane)sulfonate | | | | Adjuvant | |
| Long oil alkyd 5070 | NA 64 | NA 120 | 2648 | Insecticide | Unclassified |
| Lubrizol 544 | NA 65 | NA 121 | 2649 | Solvent, | Unclassified |
| | | | | Adjuvant | |
| Luna yellow NB-1 1270 | NA 66 | NA 122 | 2651 | Dye | Unclassified |
| Luviset cap | 25035-26-1 | NA 123 | 3867 | N/A | Unclassified |
| Macroplast UK 8202/UK 5400 | NA 57 | NA 108 | 2652 | N/A | Unclassified |
| Macroplast VAX-11688/UK | NA 58 | NA 109 | 2653 | N/A | Unclassified |
| 5400 | | | | | |
| Malachite green | 569-64-2 | 39504 | 366 | Dye | Unclassified |
| Maleic anhydride | 108-31-6 | NA 102 | 3272 | N/A | Unclassified |
| Maleic anhydride, polymer | 37199-81-8 | NA 103 | 3271 | N/A | Unclassified |
| with 2,4,4-trimethyl pentane, | | | | | |
| sodium salt | | | | | |
| Maleic hydrazide | 123-33-1 | 51501 | 368 | Plant Growth | Unclassified |
| | | | | Regulator | |
| Maleic hydrazide | 10071-13-3 | NA 210 | NA 209 | Plant Growth | Unclassified |
| | | | | Regulator | |
| Maleic hydrazide, choline salt | 71203-19-5 | 51504 | NA 422 | Plant Growth | Unclassified |
| | | | | Regulator | |
| Maleic hydrazide, | 5716-15-4 | 51502 | 778 | Plant Growth | Unclassified |
| diethanolamine salt | | | | Regulator | |
| Maleic hydrazide, potassium | 28382-15-2 | 51503 | 2130 | Plant Growth | Unclassified |
| | | | | Regulator, | |
| | | | | Herbicide | |
| Malic acid | 6915-15-7 | 51101 | 1364 | Microbiocide | Unclassified |
| Mark 1500 | NA 49 | NA 96 | 2663 | N/A | Unclassified |
| Mark 1600 | NA 50 | NA 97 | 2664 | N/A | Unclassified |
| Mark 1601 | NA 51 | NA 98 | 2665 | N/A | Unclassified |
| Mark 1603 | NA 740 | NA 135 | 2666 | N/A | Unclassified |
| Mark 1607 | NA 569 | NA 103 | 2667 | N/A | Unclassified |
| Mark 495 | NA 56 | NA 107 | 2660 | N/A | Unclassified |
| Mark 565A | NA 48 | NA 95 | 2661 | N/A | Unclassified |
| Mark 706 | NA 739 | NA 135 | 2662 | N/A | Unclassified |
| Marter white 400 | NA 52 | NA 99 | 2668 | N/A | Unclassified |
| Medicated block | NA 47 | NA 92 | 3684 | N/A | Unclassified |
| Mefenpyr | 135591-00-3 | NA 182 | NA 181 | Herbicide | Unclassified |
| | | | | Safener | |
| Mefenpyr-diethyl | 135590-91-9 | R47618 | NA 177 | Herbicide | Unclassified |
| | | | | Safener | |
| Menhaden oil | 8002-50-4 | NA 93 | 3686 | N/A | Unclassified |
| Menthol | 1490-04-6 | 51601 | 1074 | Dog and Cat | Unclassified |
| | | | | Repellent | |
| Methoxy-2,4-dihydroxy | NA 46 | NA 89 | 3281 | N/A | Unclassified |
| pentene | | | | | |
| Methoxyphenone | 41295-28-7 | NA 209 | NA 208 | Herbicide | Unclassified |
| Methyl esters of cottonseed oil | 61788-60-1 | NA 148 | 3687 | Adjuvant | Unclassified |
| Methyl ethyl ketone | 78-93-3 | 44103 | 2680 | Solvent | Unclassified |
| Methyl formate | 107-31-3 | 53701 | 391 | Fumigant | Unclassified |
| Methyl hydrogenated rosinate | 8050-15-5 | NA 82 | 3688 | N/A | Unclassified |
| Methyl isocyanate | 624-83-9 | NA 180 | NA 180 | Breakdown | Unclassified |
| | | | | product | |
| Methyl isothiocyanate | 556-61-6 | 68103 | 392 | Fumigant, | Unclassified |
| | | | | Insecticide, | |
| | | | | Herbicide, | |
| | | | | Nematicide, | |
| | | | | Breakdown | |
| | | | | product | |
| Methyl methacrylate | 80-62-6 | NA 78 | 2151 | N/A | Unclassified |
| Methylene blue | 61-73-4 | 39505 | 387 | Fungicide, Dye | Unclassified |
| Metosulam | 139528-85-1 | NA 184 | NA 184 | Herbicide | Unclassified |
| Milorganite | 68512-89-0 | NA 66 | 3693 | N/A | Unclassified |
| MKH 6561 | NA 1466 | NA 217 | NA 216 | Herbicide | Unclassified |
| MON 4660 | 71526-07-3 | ##### | NA 162 | N/A | Unclassified |
| Monosodium glutamate | 32221-81-1 | NA 59 | 2691 | N/A | Unclassified |
| Morwet EFW | NA 36 | NA 60 | 2693 | Adjuvant | Unclassified |
| Morwet IP | NA 568 | NA 102 | 3694 | Adjuvant | Unclassified |
| Morwet IP | NA 887 | NA 170 | 2694 | Adjuvant | Unclassified |
| Multiresidue carbamate screen | NA 848 | NA 158 | 4042 | N/A | Unclassified |
| Multiresidue chlorinated | NA 847 | NA 158 | 4043 | N/A | Unclassified |
| hydrocarbon screen | | | | | |
| Multiresidue organophosphate | NA 846 | NA 158 | 4044 | N/A | Unclassified |
| screen | | | | | |
| Multiresidue triazine screen | NA 845 | NA 157 | 4045 | N/A | Unclassified |
| N,N-Diallyl-2,2- | 37764-25-3 | NA 187 | NA 186 | Herbicide | Unclassified |
| dichloroacetamide | | | | Safener | |
| n-Butyl acetate | 142-62-1 | ##### | 2434 | N/A | Unclassified |
| N-methyl-2-pyrrolidone | 237900 | NA 76 | 2684 | N/A | Unclassified |
| N-TDF | NA 22 | NA 34 | 2713 | N/A | Unclassified |
| Naphthalene-formaldehyde | 9008-63-3 | NA 58 | 2692 | Insecticide, | Unclassified |
| condensate sulfonic acid, | | | | Fungicide, | |
| metallic salts | | | | Microbiocide, | |
| | | | | Adjuvant | |
| Naphthalic anhydride | 81-84-5 | NA 209 | NA 208 | Herbicide | Unclassified |
| | | | | Safener | |
| Neo-decanoic acid | 26896-20-8 | 97501 | 2699 | N/A | Unclassified |
| Neodol 25 | 63393-82-8 | NA 51 | 3930 | N/A | Unclassified |
| Neuessence powder 411669 | NA 781 | NA 140 | 3003 | N/A | Unclassified |
| Nipyraclofen | 99662-11-0 | NA 229 | NA 229 | Herbicide | Unclassified |
| Nitrile rubber | NA 743 | NA 135 | 2704 | N/A | Unclassified |
| Nitro ethane | 79-24-3 | NA 49 | 3298 | Solvent | Unclassified |
| Nitromethane | 75-52-5 | NA 42 | 3299 | Solvent | Unclassified |
| Nitrothal-isopropyl | 10552-74-6 | NA 181 | NA 180 | Fungicide | Unclassified |
| Non-ionic surfactants | NA 1443 | NA 214 | NA 213 | Soap/Surfactant, | Unclassified |
| | | | | Adjuvant | |
| Nonoxynol-9-phosphate | NA 29 | NA 43 | 2706 | Adjuvant | Unclassified |
| Norbormide | 991-42-4 | 86201 | 2711 | N/A | Unclassified |
| Nuostabe v-1913 | NA 23 | NA 35 | 2715 | N/A | Unclassified |
| Nuxtra calcium 6% catalyst | NA 25 | NA 37 | 2716 | N/A | Unclassified |
| Nuxtra calcium 8% catalyst | NA 26 | NA 38 | 2717 | N/A | Unclassified |
| Nuxtra manganese 9% catalyst | NA 15 | NA 24 | 2718 | N/A | Unclassified |
| Nylon | 32131-17-2 | NA 25 | 3703 | N/A | Unclassified |
| Octachlorostyrene | 29082-74-4 | NA 215 | NA 215 | N/A | Unclassified |
| Orvus ES paste concentrate | NA 3 | NA 3 | 2726 | N/A | Unclassified |
| Ovex | 80-33-1 | 20201 | 452 | Insect Growth | Unclassified |
| | | | | Regulator | |
| Oxabetrinil | 74782-23-3 | NA 205 | NA 204 | Herbicide | Unclassified |
| | | | | Safener | |
| Oxadiargyl | 39807-15-3 | NA 211 | NA 211 | Herbicide | Unclassified |
| Oxapyrazon | 4489-31-0 | NA 209 | NA 208 | Herbicide | Unclassified |
| Oxapyrazon sodium | 25316-56-7 | NA 217 | NA 217 | Herbicide | Unclassified |
| Oxaziclomefone | 153197-14-9 | NA 233 | NA 233 | Herbicide | Unclassified |
| Oxidized polyethylene | 68441-17-8 | NA 118 | 3713 | N/A | Unclassified |
| Oxygenated solvents | NA 379 | NA 687 | 1767 | Solvent | Unclassified |
| p-Chloronitrobenzene | 100-00-5 | NA 183 | NA 182 | Insecticide | Unclassified |
| Paint | NA 380 | NA 688 | 3715 | N/A | Unclassified |
| Panasol AN-2 | NA 381 | NA 690 | 2733 | Solvent | Unclassified |
| Panasol AN-3 | NA 382 | NA 691 | 2732 | Solvent | Unclassified |
| Paraformaldehyde | 30525-89-4 | 43002 | 456 | Microbiocide, | Unclassified |
| | | | | Fungicide, | |
| | | | | Insecticide | |
| Pareth 91-8 | NA 889 | NA 170 | 2734 | N/A | Unclassified |
| Pentoxazone | 110956-75-7 | NA 233 | NA 233 | Herbicide | Unclassified |
| Perfluorooctane sulfonate | NA 1456 | NA 216 | NA 215 | Insecticide | Unclassified |
| Perfume 08-28 | NA 750 | NA 136 | 2740 | Fragrance | Unclassified |
| Perfume bouquet no. 3 | NA 366 | NA 672 | 2741 | Fragrance | Unclassified |
| Perfume, chem-mask C no. 280 | NA 367 | NA 673 | 2742 | Fragrance | Unclassified |
| Perfume, colonial bouquet | NA 368 | NA 674 | 2744 | Fragrance | Unclassified |
| 8095 | | | | | |
| Perfume, compound 2-2723 | NA 752 | NA 136 | 2745 | Fragrance | Unclassified |
| Perfume, compound 44.368/g | NA 370 | NA 676 | 2747 | Fragrance | Unclassified |
| Perfume, compound bouquet | NA 369 | NA 675 | 2746 | Fragrance | Unclassified |
| EC | | | | | |
| Perfume, DL 10613 | NA 823 | NA 151 | 3888 | Fragrance | Unclassified |
| Perfume, eaudesol mal | NA 371 | NA 677 | 2748 | Fragrance | Unclassified |
| (special) | | | | | |
| Perfume, fragrance oil 6198 | NA 372 | NA 678 | 2750 | Fragrance | Unclassified |
| AG | | | | | |
| Perfume, fresh meadows #651 | NA 753 | NA 136 | 2751 | Fragrance | Unclassified |
| Perfume, herbal fragrance oil | NA 354 | NA 657 | 2753 | Fragrance | Unclassified |
| no. 062 | | | | | |
| Perfume, malamasque no. 59 | NA 355 | NA 658 | 2754 | Fragrance | Unclassified |
| Perfume, malathion 21-639 | NA 356 | NA 659 | 2755 | Fragrance | Unclassified |
| Perfume, marbelle 72160A | NA 357 | NA 660 | 2756 | Fragrance | Unclassified |
| Perfume, mimosa no. 5 | NA 754 | NA 136 | 2757 | Fragrance | Unclassified |
| Perfume, neutroleum alpha | NA 358 | NA 661 | 2758 | Fragrance | Unclassified |
| Perfume, new mown hay | NA 359 | NA 662 | 2759 | Fragrance | Unclassified |
| Perfume, norda EC-137 | NA 360 | NA 663 | 2760 | Fragrance | Unclassified |
| Perfume, pet perfume # 27253 | NA 361 | NA 664 | 2761 | Fragrance | Unclassified |
| Perfume, phase b-5 | NA 755 | NA 136 | 2762 | Fragrance | Unclassified |
| Perfume, R-7402 | NA 362 | NA 665 | 2763 | Fragrance | Unclassified |
| Perfume, veilex 40019 | NA 363 | NA 666 | 2764 | Fragrance | Unclassified |
| Perfume, washroom cleaner | NA 364 | NA 667 | 2765 | Fragrance | Unclassified |
| bouquet # 152-587 | | | | | |
| Phenyl ether | 101-84-8 | NA 653 | 3324 | N/A | Unclassified |
| Phosametine | NA 1400 | NA 197 | NA 196 | Herbicide | Unclassified |
| Phosdiphen | 36519-00-3 | NA 209 | NA 209 | Fungicide | Unclassified |
| Phosphoproteins | NA 350 | NA 646 | 1250 | N/A | Unclassified |
| Phthalaldehyde | 643-79-8 | ##### | 5107 | Microbiocide | Unclassified |
| Phthalide | 27355-22-2 | NA 205 | NA 205 | Fungicide | Unclassified |
| Phthalo green | NA 348 | NA 642 | 2779 | Dye | Unclassified |
| Picaridin | NA 1434 | NA 212 | NA 211 | Insect | Unclassified |
| | | | | Repellent | |
| Pigment blue 29 | 57455-37-5 | NA 643 | 2780 | Dye | Unclassified |
| Pigment green 7 | 1328-53-6 | NA 636 | 3870 | Dye | Unclassified |
| Pigment red 178 | 3049-71-6 | NA 637 | 3869 | Dye | Unclassified |
| Pigment red 57:1 | 6887-51-9 | NA 171 | 3868 | Dye | Unclassified |
| Pigment red no. 48 | 16013-44-8 | NA 149 | 3729 | Dye | Unclassified |
| Pimaricin | 7681-93-8 | NA 203 | NA 202 | Fungicide | Unclassified |
| Piperalin | 3478-94-2 | 97003 | 488 | Fungicide | Unclassified |
| Piperine | 94-62-2 | NA 223 | NA 223 | N/A | Unclassified |
| Piproctanyl | 69309-47-3 | NA 209 | NA 209 | Plant Growth | Unclassified |
| | | | | Regulator | |
| Piproctanyl bromide | 56717-11-4 | NA 218 | NA 217 | Plant Growth | Unclassified |
| | | | | Regulator | |
| Plastic polymers | NA 339 | NA 625 | 764 | Adjuvant | Unclassified |
| Plurafac B-26 | 69227-21-0 | NA 626 | 2782 | N/A | Unclassified |
| Plurafac C-17 | 69227-21-0 | NA 137 | 2783 | N/A | Unclassified |
| Pluraflo E4A | NA 340 | NA 627 | 2784 | Soap/Surfactant | Unclassified |
| Pluronic 10-R-5 | NA 345 | NA 632 | 2790 | N/A | Unclassified |
| Pluronic 1-101 | NA 759 | NA 137 | 2788 | N/A | Unclassified |
| Pluronic L-62 LF | NA 342 | NA 629 | 2786 | N/A | Unclassified |
| Pluronic L61 | NA 341 | NA 628 | 2785 | N/A | Unclassified |
| Pluronic 163 | NA 343 | NA 630 | 2787 | N/A | Unclassified |
| Pluronic P-104 | NA 344 | NA 631 | 2789 | N/A | Unclassified |
| PMS-5406-T pigment | NA 346 | NA 633 | 2791 | N/A | Unclassified |
| Po-san | 37339-61-0 | 98804 | NA 861 | N/A | Unclassified |
| Po-san | 76930-44-4 | 98804 | NA 862 | N/A | Unclassified |
| Poly(oxyethylene) | 31512-74-0 | 69183 | 1576 | Microbiocide | Unclassified |
| (dimethylimino) ethylene | | | | | |
| (dimethylimino) ethylene | | | | | |
| dichloride | | | | | |
| Poly-i-para-menthene | 34363-01-4 | NA 610 | 1314 | Insect Growth | Unclassified |
| | | | | Regulator | |
| Polyacrylamide polymer | 2592984 | NA 634 | 2111 | Adjuvant | Unclassified |
| Polyalkylene ether | NA 347 | NA 635 | 2271 | Adjuvant | Unclassified |
| Polyamide resin | NA 621 | NA 112 | 2795 | N/A | Unclassified |
| Polyamine polymer | NA 334 | NA 620 | 1907 | Adjuvant | Unclassified |
| Polyhexamethylene | 32289-58-0 | ##### | 2258 | Microbiocide, | Unclassified |
| biguanidine | | | | Fungicide | |
| Polymer emulsion K | NA 329 | NA 611 | 2804 | Adjuvant | Unclassified |
| Polymerized acrylic acid | 2592860 | 52902 | 3738 | Adjuvant | Unclassified |
| Polyoxin | 11113-80-7 | NA 194 | NA 193 | Fungicide | Unclassified |
| Polyurethane | NA 426 | NA 768 | 3770 | N/A | Unclassified |
| Polyvinyl butyral resin | NA 427 | NA 769 | 2820 | N/A | Unclassified |
| Polyvinyl chloride | 9002-86-2 | NA 770 | 2821 | N/A | Unclassified |
| Polyvinylpyrrolidone- | 82010-83-1 | 43003 | NA 367 | Microbiocide | Unclassified |
| formaldehyde complex | | | | | |
| Polyvis O-SH | NA 428 | NA 771 | 2823 | N/A | Unclassified |
| Poly[hydroxyethylene(dimethyliminio) | NA 1512 | NA 223 | NA 222 | Microbiocide | Unclassified |
| ethylene(dimethylimino) | | | | | |
| methylene dichloride] | | | | | |
| Potassium 3-(2-(2-((2-(2- | NA 998 | NA 779 | 3352 | Preservative | Unclassified |
| hydroxy ethyl) ethyl) octadecyl | | | | | |
| amino)ethoxy) propionate | | | | | |
| Potassium N-ethyl perfluoro | NA 997 | NA 777 | 3354 | Insecticide, | Unclassified |
| octane sulfonamide acetate | | | | Adjuvant | |
| Probenazole | 27605-76-1 | NA 205 | NA 205 | Fungicide, | Unclassified |
| | | | | Microbiocide | |
| Propellant A-46 | NA 441 | NA 794 | 2834 | Propellant | Unclassified |
| Propellant A-70 | NA 762 | NA 137 | 2835 | Propellant | Unclassified |
| Propellant A-91 | NA 442 | NA 795 | 2836 | Propellant | Unclassified |
| Propellant B-46 | NA 443 | NA 796 | 3887 | Propellant | Unclassified |
| Propidine | NA 1449 | NA 215 | NA 214 | Insect | Unclassified |
| | | | | Repellent | |
| Propionic acid | 79-09-4 | 77702 | 505 | Fungicide, | Unclassified |
| | | | | Microbiocide, | |
| | | | | Preservative | |
| Propyl-3-t-butylphenoxyacetate | NA 1407 | NA 198 | NA 197 | Plant Growth | Unclassified |
| | | | | Regulator | |
| Pyrazolynate | 58011-68-0 | NA 205 | NA 205 | Herbicide | Unclassified |
| Pyribenzoxim | 168088-61-7 | NA 233 | NA 233 | Herbicide | Unclassified |
| Pyriminobac | 136191-56-5 | NA 206 | NA 205 | Herbicide | Unclassified |
| Pyroquilone | 57369-32-1 | NA 190 | NA 189 | Fungicide | Unclassified |
| Quinacetol sulfate | 57130-91-3 | NA 210 | NA 209 | Fungicide | Unclassified |
| Quinizarin green SS | 128-80-3 | NA 109 | 3374 | Dye | Unclassified |
| Quinoclamine | 2797-51-5 | NA 185 | NA 184 | Herbicide, | Unclassified |
| | | | | Algaecide | |
| Quinoxyfen | 124495-18-7 | NA 206 | NA 205 | Fungicide | Unclassified |
| Resins and polymers | NA 1374 | NA 194 | NA 193 | Pruning Aid, | Unclassified |
| | | | | Adjuvant | |
| RH - 2485 | NA 1467 | NA 217 | NA 217 | Insecticide | Unclassified |
| Rhodamine B | 81-88-9 | NA 138 | 2846 | Dye | Unclassified |
| Sebacic acid | 111-20-6 | NA 187 | NA 186 | Repellent | Unclassified |
| Sec-butylamine | 13952-84-6 | 4214 | 1704 | Fungicide | Unclassified |
| Seconal | 76-73-3 | NA 190 | NA 189 | N/A | Unclassified |
| Secret formula no. 1 | NA 2 | NA 2 | 1837 | N/A | Unclassified, |
| | | | | | Unclassified |
| Secret formula no. 2 | NA 458 | NA 820 | 1838 | N/A | Unclassified |
| Silthiofam | 175217-20-6 | NA 210 | NA 210 | Fungicide | Unclassified |
| Sintofen | 130561-48-7 | NA 185 | NA 184 | Plant Growth | Unclassified |
| | | | | Regulator | |
| Sodium 2-ethylhexanoate | 19766-89-3 | NA 214 | NA 214 | Fungicide, | Unclassified |
| | | | | Microbiocide | |
| Sodium caprylate | 29372 | 79063 | 3388 | N/A | Unclassified |
| Sodium chloroacetate | 3926-62-3 | ##### | NA 141 | Herbicide | Unclassified |
| Sodium diacetoneketogulonate | NA 1397 | NA 197 | NA 196 | Plant Growth | Unclassified |
| | | | | Regulator | |
| Sodium gluconate | 527-07-1 | 105 | 3406 | N/A | Unclassified |
| Sodium methyl sulfate | 512-42-5 | NA 145 | 3417 | N/A | Unclassified |
| Sodium petroleum sulfonate | 68608-26-4 | 79035 | 3431 | Insecticide, | Unclassified |
| | | | | Adjuvant | |
| Sodium propionate | 137-40-6 | 77703 | 3434 | Fungicide, | Unclassified |
| | | | | Preservative | |
| Solulan 98 | NA 474 | NA 874 | 2867 | Solvent | Unclassified |
| Solulan L-575 | NA 763 | NA 138 | 2868 | N/A | Unclassified |
| Solvent red 49 | 509-34-2 | NA 875 | 3900 | Dye | Unclassified |
| Spiroxamine | 118134-30-8 | NA 202 | NA 202 | Fungicide | Unclassified |
| Sponto 168 | NA 484 | NA 888 | 2875 | N/A | Unclassified |
| Sponto 232T | NA 492 | NA 896 | 2885 | N/A | Unclassified |
| Sponto 234T | NA 766 | NA 138 | 2886 | N/A | Unclassified |
| Sponto AK-30-02BT | NA 485 | NA 889 | 2876 | N/A | Unclassified |
| Sponto AK31-27A | NA 764 | NA 138 | 2877 | N/A | Unclassified |
| Sponto AK31-27B | NA 486 | NA 890 | 2878 | N/A | Unclassified |
| Sponto AK31-38 | NA 487 | NA 891 | 2879 | N/A | Unclassified |
| Sponto AK31-56A | NA 488 | NA 892 | 2880 | N/A | Unclassified |
| Sponto AK31-56B | NA 765 | NA 138 | 2881 | N/A | Unclassified |
| Sponto AK31-56M | NA 632 | NA 116 | 3558 | N/A | Unclassified |
| Sponto N-140B | NA 489 | NA 893 | 2882 | N/A | Unclassified |
| Sponto N-300B | NA 490 | NA 894 | 2883 | N/A | Unclassified |
| Sponto N-500B | NA 491 | NA 895 | 2884 | N/A | Unclassified |
| Sta-pro 3000 | NA 493 | NA 897 | 2889 | N/A | Unclassified |
| Stepan C-65 methyl ester | NA 496 | NA 903 | 2893 | N/A | Unclassified |
| Stepantan A | NA 497 | NA 904 | 2894 | N/A | Unclassified |
| Stepfac 8172 | NA 767 | NA 138 | 2895 | N/A | Unclassified |
| Sterilix | NA 656 | NA 121 | 550 | Microbiocide | Unclassified |
| Sterox NJ | NA 498 | NA 905 | 2896 | N/A | Unclassified |
| Stoddard solvent | 8052-41-3 | 63504 | NA 516 | Solvent, | Unclassified |
| | | | | Herbicide, | |
| | | | | Insecticide | |
| Styrene acrylic copolymer | 25085-34-1 | NA 907 | 3457 | Adjuvant | Unclassified |
| Styrene/pvp copolymer | 25086-29-7 | NA 908 | 3812 | Adjuvant | Unclassified |
| Sudan III | 85-86-9 | NA 909 | 3459 | Dye | Unclassified |
| Sulcotrione | 99105-77-8 | NA 185 | NA 184 | Herbicide | Unclassified |
| Sulfated dipentane | NA 500 | NA 911 | 3460 | N/A | Unclassified |
| Sulfonated cod liver oil | NA 1464 | NA 217 | NA 216 | N/A | Unclassified |
| Sulglycapin | 51068-60-1 | NA 233 | NA 233 | Herbicide | Unclassified |
| Sure sol 180 | NA 505 | NA 916 | 2903 | Solvent | Unclassified |
| Surflo B11 | NA 606 | NA 110 | 2904 | Soap/Surfactant | Unclassified |
| Surflo B13 | NA 506 | NA 917 | 2905 | Soap/Surfactant | Unclassified |
| Surflo B16 | NA 507 | NA 918 | 2906 | Soap/Surfactant | Unclassified |
| Surflo B17 | NA 508 | NA 919 | 2907 | Soap/Surfactant | Unclassified |
| Surflo B19 | NA 770 | NA 139 | 2908 | Soap/Surfactant | Unclassified |
| Sustane 3 | NA 622 | NA 113 | 2909 | N/A | Unclassified |
| T-DETN-9.5 | NA 513 | NA 928 | 2917 | N/A | Unclassified |
| T-mulz 565 | NA 528 | NA 955 | 2944 | Adjuvant | Unclassified |
| T-mulz 94W | NA 531 | NA 958 | 2949 | Adjuvant | Unclassified |
| T-mulz AO2 | NA 772 | NA 139 | 2943 | N/A | Unclassified |
| T-mulz O | NA 529 | NA 956 | 2945 | Adjuvant | Unclassified |
| T-mulz PB | NA 530 | NA 957 | 2946 | Adjuvant | Unclassified |
| T-Mulz VO | NA 610 | NA 111 | 2947 | N/A | Unclassified |
| T-mulz W | NA 773 | NA 139 | 2948 | N/A | Unclassified |
| Tabu powder | NA 510 | NA 922 | 1462 | N/A | Unclassified |
| Tacks | NA 818 | NA 151 | 3816 | N/A | Unclassified |
| Tenneco 225F PVC resin | NA 514 | NA 930 | 2919 | N/A | Unclassified |
| Tenneco T500-100 | NA 771 | NA 139 | 2920 | N/A | Unclassified |
| Tenox 2 | NA 515 | NA 931 | 2921 | Preservative | Unclassified |
| Tenox 4 | NA 516 | NA 932 | 2922 | Preservative | Unclassified |
| Tenox R | NA 517 | NA 933 | 2923 | Preservative | Unclassified |
| Tenox S-1 | NA 518 | NA 934 | 2924 | Preservative | Unclassified |
| Tensiofix B7416 | NA 895 | NA 171 | 3943 | Soap/Surfactant | Unclassified |
| Tensiofix B7453 | NA 896 | NA 171 | 3944 | Soap/Surfactant | Unclassified |
| Tergitol 15-S-12 | NA 623 | NA 113 | 2926 | N/A | Unclassified |
| Tergitol 15-S-12 acrylate | NA 519 | NA 935 | 2927 | N/A | Unclassified |
| Tergitol 15-S-20 | NA 520 | NA 936 | 2928 | N/A | Unclassified |
| tert-Butylamine Endothall | 26648-01-1 | 38909 | NA 327 | Herbicide, | Unclassified |
| | | | | Defoliant | |
| Tetrahydrofuran | 109-99-9 | NA 944 | 2933 | Solvent | Unclassified |
| Texaphor special | NA 524 | NA 951 | 2935 | N/A | Unclassified |
| TF-310 | NA 525 | NA 952 | 3524 | N/A | Unclassified |
| Thanite | 115-31-1 | 47101 | 586 | Insecticide | Unclassified |
| Thickener, unknown | NA 526 | NA 953 | 2936 | Adjuvant | Unclassified |
| Thicyofen | 116170-30-0 | NA 210 | NA 209 | Fungicide | Unclassified |
| Thidiazimin | 123249-43-4 | NA 230 | NA 229 | Herbicide | Unclassified |
| Thiocyclam | 31895-21-3 | NA 182 | NA 181 | Insecticide | Unclassified |
| Thiocyclam hydrogen oxalate | 31895-22-4 | ##### | NA 104 | Insecticide | Unclassified |
| Thixatrol ST | NA 820 | NA 151 | 3853 | N/A | Unclassified |
| Toximul 500 | NA 532 | NA 963 | 2951 | Insecticide | Unclassified |
| Toximul 850 m | NA 826 | NA 152 | 3918 | N/A | Unclassified |
| Toximul D | NA 533 | NA 964 | 2952 | N/A | Unclassified |
| Toximul H | NA 534 | NA 965 | 2953 | N/A | Unclassified |
| Toximul P | NA 774 | NA 140 | 2954 | N/A | Unclassified |
| Toximul TA-5 | NA 535 | NA 966 | 2955 | N/A | Unclassified |
| Triapenthenol | 76608-88-3 | NA 190 | NA 189 | Plant Growth | Unclassified |
| | | | | Regulator | |
| Triaryl methane | NA 536 | NA 967 | 2957 | N/A | Unclassified |
| Triazoxide | 72459-58-6 | NA 185 | NA 184 | Fungicide | Unclassified |
| Tributyl phosphate | 126-73-8 | NA 969 | 3480 | N/A | Unclassified |
| Trichlamide | 70193-21-4 | NA 210 | NA 209 | Fungicide | Unclassified |
| Trichloropyridinol | NA 1338 | NA 180 | NA 179 | Breakdown | Unclassified |
| | | | | product | |
| Tridemorph | 24602-86-6 | ##### | NA 987 | Fungicide | Unclassified |
| Tridemorph | 81412-43-3 | ##### | NA 201 | Fungicide | Unclassified |
| Triethyl phosphate | 78-40-0 | NA 979 | 3489 | N/A | Unclassified |
| Trioxymethylene | 110-88-3 | NA 190 | NA 189 | Microbiocide, | Unclassified |
| | | | | Fungicide | |
| Triton CF-10 | NA 542 | NA 991 | 2968 | Soap/Surfactant | Unclassified |
| Triton X-363 | NA 612 | NA 111 | 2969 | Soap/Surfactant | Unclassified |
| Trycol NP-4 | NA 775 | NA 140 | 2970 | N/A | Unclassified |
| Trycol NP-6 | NA 543 | NA 993 | 2971 | N/A | Unclassified |
| Trycol NP-9 | NA 544 | NA 994 | 2972 | N/A | Unclassified |
| Tryfac 5566 | NA 545 | NA 995 | 2973 | N/A | Unclassified |
| Tryfac 910-K | NA 546 | NA 996 | 2974 | N/A | Unclassified |
| Turgasept | NA 776 | NA 140 | 2975 | N/A | Unclassified |
| Ucar solution vinyl VAGH | NA 548 | NA 999 | 2976 | N/A | Unclassified |
| UL-94 HF1 listed polyester | NA 549 | NA 100 | 2977 | N/A | Unclassified |
| foam | | | | | |
| Unknown (In EPA Product | NA 1175 | 67323 | NA 165 | Fungicide | Unclassified |
| data w/o name) | | | | | |
| Unknown (In EPA Product | NA 1176 | 69144 | NA 165 | Microbiocide | Unclassified |
| data w/o name) | | | | | |
| Unknown (In EPA Product | NA 1177 | 69162 | NA 165 | Microbiocide | Unclassified |
| data w/o name) | | | | | |
| Unknown (In EPA Product | NA 1178 | 79061 | NA 165 | Microbiocide | Unclassified |
| data w/o name) | | | | | |
| Unknown (In EPA Product | NA 1179 | ##### | NA 166 | Microbiocide | Unclassified |
| data w/o name) | | | | | |
| Unknown (In EPA Product | NA 1180 | ##### | NA 166 | Microbiocide | Unclassified |
| data w/o name) | | | | | |
| Unknown (In EPA Product | NA 1181 | ##### | NA 166 | Microbiocide | Unclassified |
| data w/o name) | | | | | |
| Unknown (In EPA Product | NA 1182 | ##### | NA 166 | N/A | Unclassified |
| data w/o name) | | | | | |
| Unknown (In EPA Product | NA 1183 | ##### | NA 166 | N/A | Unclassified |
| data w/o name) | | | | | |
| Urethane adhesive, parts a & b | NA 551 | NA 100 | 2979 | N/A | Unclassified |
| Versalon 1112 | NA 613 | NA 111 | 2984 | N/A | Unclassified |
| Versamid resins | NA 651 | NA 119 | 3822 | N/A | Unclassified |
| Vinyl acetate | 108-05-4 | NA 100 | 2116 | N/A | Unclassified |
| Vinyl polymer | NA 680 | NA 125 | 1754 | Adjuvant | Unclassified |
| Vinyl resin, synthetic | NA 554 | NA 100 | 1533 | N/A | Unclassified |
| Vitamin K3 | 58-27-5 | 6319 | NA 89 | N/A | Unclassified |
| Water soluble dyes | NA 1517 | NA 223 | NA 223 | Dye | Unclassified |
| Waxes | NA 1376 | NA 194 | NA 194 | Pruning Aid, | Unclassified |
| | | | | Adjuvant | |
| Wickenol 535 | NA 779 | NA 140 | 2989 | N/A | Unclassified |
| Wing stay V | NA 561 | NA 101 | 2990 | N/A | Unclassified |
| Witafrol 7420 | NA 625 | NA 113 | 2991 | N/A | Unclassified |
| Witcamine tam-45 | NA 562 | NA 101 | 2992 | N/A | Unclassified |
| Witco C-5752 | NA 563 | NA 101 | 3882 | N/A | Unclassified |
| Witconate p-1020bu | NA 614 | NA 111 | 3884 | N/A | Unclassified |
| Witconol 1S-108 | NA 564 | NA 101 | 2993 | N/A | Unclassified |
| Zein | 9010-66-6 | NA 102 | 3504 | N/A | Unclassified |

*N/A = Not Available\

### II. Augmenting the Sensitivity of Plants to Herbicides

In certain embodiments of the invention, the effectiveness of herbicides is augmented by methods and compositions for the contemporaneous administration of one or more ectophosphatase inhibitors in combination with herbicides. Such applications may, in addition to increasing the effectiveness of herbicides, allow the use of reduced concentrations of herbicides, thereby providing cost and environmental benefits.

Specifically contemplated by the invention are compositions comprising one or more ectophosphatase inhibitor(s) in combination with one or more herbicide(s) listed in Table 1. In one embodiment of the invention, the ectophosphatase inhibitor may or may not be a compound selected from formula I-XX. The herbicidal compositions or other compositions formulated for agricultural use, including compositions comprising insecticides and growth regulators, may be used with potentially any plant. The term “plant,” as used herein, refers to any type of plant. The inventors have provided below an exemplary description of some plants that may be used with the invention. However, the list is provided for illustrative purposes only and is not limiting, as other types of plants will be known to those of skill in the art and could be used with the invention.

Non-limiting examples of plant genera that may be treated in accordance with the invention include: Abutilon, Amaranthus, Artemisia, Asclepias, Avena, Axonopus, Borreria, Brachiaria, Brassica, Bromus, Chenopodium, Cirsium, Commelina, Convolvulus, Cynodon, Cyperus, Digitaria, Echinochloa, Eleusine, Elymus, Equisetum, Erodium, Helianthus, Imperata, Ipomoea, Kochia, Lolium, Malva, Oryza, Ottochloa, Panicum, Paspalum, Phalaris, Phragmites, Polygonum, Portulaca, Pteridium, Pueraria, Rubus, Salsola, Setaria, Sida, Sinapis, Sorghum, Triticum, Typha, Ulex, Xanthium, and Zea. Non-limiting common types of plants that may be controlled in accordance with the invention include varieties of grasses, broad leafs, succulents, trees and shrubs, barley, black nightshade, broadleaf signal grass, burcumber, chickweed, common ragweed, crabgrass, field pennycress, rough fleabane, foxtail, giant ragweed, goose grass, groundcherry, hemp sesbarria, henbit, jungle rice, kochia, lambs quarters, morning-glory spp., mustard, fall and Texas panicum, palma amaranth, prickly sida (teaweed), red rice, rye, seedling shattercone, shepherd's purse, sicklepod, sprangletop, sunflower, velvet leaf, volunteer corn, common and tall waterhemp, wheat, wild proso millet, witchgrass, wolly cupgrass; and common perennial weeds including Canada thistle, common milkweed, field bindweed, hemp dogbane, red vine, rhizone johnson grass, tall fescue, trumpet creeper, swamp smartweed and wisteria mukly. Star Thistle, Poison Oak, and Ivy.

Non-limiting examples of species that may be controlled include velvetleaf (Abutilon theophrasti), pigweed (Amaranthus spp.), buttonweed (Borreria spp.), oilseed rape, canola, indian mustard, etc. (Brassica spp.), commelina (Commelina spp.), filaree (Erodium spp.), sunflower (Helianthus spp.), morningglory (Ipomoea spp.), kochia (Kochia scoparia), mallow (Malva spp.), wild buckwheat, smartweed, etc. (Polygonum spp.), purslane (Portulaca spp.), russian thistle (Salsola spp.), sida (Sida spp.), wild mustard (Sinapis arvensis), cocklebur (Xanthium spp.), wild oat (Avena fatua), carpetgrass (Axonopus spp.), downy brome (Bromus tectorum), crabgrass (Digitaria spp.), barnyardgrass (Echinochloa crus-galli), goosegrass (Eleusine indica), annual ryegrass (Lolium multiflorum), rice (Oryza sativa), ottochloa (Ottochloa nodosa), bahiagrass (Paspalum notatum), canarygrass (Phalaris spp.), foxtail (Setania spp.), wheat (Triticum aestivum), corn (Zea mays), mugwort (Artemisia spp.), milkweed (Asclepias spp.), canada thistle (Cirsium arvense), field bindweed (Convolvulus arvensis), kudzu (Pueraria spp.), brachiaria (Brachiaria spp.), bermudagrass (Cynodon dactylon), yellow nutsedge (Cyperus esculentus), purple nutsedge (C. rotundus), quackgrass (Elymus repens), lalang (Imperata cylindrica), perennial ryegrass (Lolium perenne), guineagrass (Panicum maximum), dallisgrass (Paspalum dilatatum), reed (Phragmites spp.), johnsongrass (Sorghum halepense), cattail (Typha spp.), horsetail (Equisetum spp.), bracken (Pteridium aquilinum), blackberry (Rubus spp.) and gorse (Ulex europaeus).

Crop and other cultivated species may also be contacted with a composition of the invention. Non-limiting examples of cultivated plants include, but are not limited to, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Helianthus, Lactuca, Bromus, Asparagus, Antirrhinum, Hemerocallis, Nemesia, Pelargonium, Panicum, Pennisetum, Ranunculus, Senecio, Salpiglossis, Cucumis, Bromelia, Glycine, Lolium, Zea, Triticum, Sorghum, Ipomoea, Passifora, Cyclamen, Malus, Prumus, Rosa, Rubus, Populus, Santalum, Allium, Lilium, Narcissus, Ananas, Arachis, Phaseolus, Pisum, Oryza, Hordeum, Gossypium.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet and fodder beet), sweet potatoes, swiss-chard, horseradish, tomatoes, kale, turnips, and spices.

Other types of plants frequently finding commercial use include fruit and vine crops such as apples, apricots, cherries, nectarines, peaches, pears, plums, prunes, quince almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, and lychee.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts), fiber plants (cotton, flax, hemp, jute), lauraceae (cinnamon, camphor), or plants such as coffee, sugarcane, tea, and natural rubber plants.

Another economically important group of plants are ornamental plants. Examples of commonly grown ornamental plants include alstroemeria (e.g., Alstoemeria brasiliensis), aster, azalea (e.g., Rhododendron sp.), begonias (e.g., Begonia sp.), bellflower, bouganvillea, cactus (e.g., Cactaceae schlumbergera truncata), camellia, carnation (e.g., Dianthus caryophyllus), chrysanthemums (e.g., Chrysanthemum sp.), clematis (e.g., Clematis sp.), cockscomb, columbine, cyclamen (e.g., Cyclamen sp.), daffodils (e.g., Narcissus sp.), false cypress, freesia (e.g., Freesia refracta), geraniums, gerberas, gladiolus (e.g., Gladiolus sp.), holly, hybiscus (e.g., Hibiscus rosasanensis), hydrangea (e.g., Macrophylla hydrangea), juniper, lilies (e.g., Lilium sp.), magnolia, miniroses, orchids (e.g., members of the family Orchidaceae), petunias (e.g., Petunia hybrida), poinsettia (e.g., Euphorbia pulcherima), primroses, rhododendron, roses (e.g., Rosa sp.), snapdragons (e.g., Antirrhinum sp.), shrubs, trees such as forest (broad-leaved trees and evergreens, such as conifers) and tulips (e.g., Tulipa sp.).

### III. Antibiotics

Another embodiment of the invention concerns methods and compositions for the contemporaneous administration of ectophosphatase inhibitors and antibiotics. Antibiotics are any chemical of natural or synthetic origin that kill or inhibit the growth of other types cells. Many clinically-useful antibiotics are produced by microorganisms. Antibiotics are typically low molecular-weight (non-protein) molecules produced as secondary metabolites, mainly by microorganisms that live in the soil. Most of these microorganisms form some type of a spore or other dormant cell, and there is thought to be some relationship (besides temporal) between antibiotic production and the processes of sporulation. Among molds, the notable antibiotic producers are Penicillium and Cephalosporium, which are the main source of the beta-lactam antibiotics (penicillin and its relatives). In the Bacteria, the Actinomycetes, notably Streptomyces species, produce a variety of types of antibiotics including the aminoglycosides (e.g. streptomycin), macrolides (e.g. erythromycin), and the tetracyclines. Endospore-forming Bacillus species produce polypeptide antibiotics such as polymyxin and bacitracin. The table below (Table 1) is a summary of the classes of antibiotics and their properties including their biological sources.

As set forth in Table 2, certain aspects of the invention concern compositions comprising an ectophosphatase inhibitor and an antibiotic of a class selected from the group consisting of: beta-lactams (penicillins and cephalosporins); semisynthetic penicillin; clavulanic Acid; monobactams; carboxypenems; aminoglycosides; glycopeptides; lincomycins; macrolides; polypeptides; chloramphenicol; polyenes; rifamycins; tetracyclines; and semisynthetic tetracycline. Any ectophosphatase inhibitor may be used in conjuntion with these classes of antibiotics. In certain embodiments of the invention, the ectophosphatase inhibitor is selected from the group consisting of the compounds of formulas I-XX.

TABLE 2
Classes of antibiotics and their properties

| | | | Spectrum (effective | |
|---|---|---|---|---|
| Chemical class | Examples | Biological source | against) | Mode of action |
| Beta-lactams | Penicillin G, | Penicillium notatum and | Gram-positive bacteria | Inhibits steps in cell wall |
| (penicillins and | Cephalothin | Cephalosporium species | | (peptidoglycan) synthesis and |
| cephalosporins) | | | | murein assembly |
| Semisynthetic | Ampicillin, | | Gram-positive and Gram- | Inhibits steps in cell wall |
| penicillin | Amoxycillin | | negative bacteria | (peptidoglycan) synthesis and |
| | | | | murein assembly |
| Clavulanic Acid | Clavamox is | Streptomyces clavuligerus | Gram-positive and Gram- | Suicide inhibitor of beta-lactamases |
| | clavulanic acid plus | | negative bacteria | |
| | amoxycillin | | | |
| Monobactams | Aztreonam | Chromobacter violaceum | Gram-positive and Gram- | Inhibits steps in cell wall |
| | | | negative bacteria | (peptidoglycan) synthesis and |
| | | | | murein assembly |
| Carboxypenems | Imipenem | Streptomyces cattleya | Gram-positive and Gram- | Inhibits steps in cell wall |
| | | | negative bacteria | (peptidoglycan) synthesis and |
| | | | | murein assembly |
| Aminoglycosides | Streptomycin | Streptomyces griseus | Gram-positive and Gram- | Inhibit translation (protein synthesis) |
| | | | negative bacteria | |
| | Gentamicin | Micromonospora species | Gram-positive and Gram- | Inhibit translation (protein synthesis) |
| | | | negative bacteria esp. | |
| | | | Pseudomonas | |
| Glycopeptides | Vancomycin | Streptomyces orientales | Gram-positive bacteria, | Inhibits steps in murein |
| | | | esp. Staphylococcus | (peptidoglycan) biosynthesis and |
| | | | aureus | assembly |
| Lincomycins | Clindamycin | Streptomyces lincolnensis | Gram-positive and Gram- | Inhibits translation (protein |
| | | | negative bacteria esp. | synthesis) |
| | | | anaerobic Bacteroides | |
| Macrolides | Erythromycin | Streptomyces erythreus | Gram-positive bacteria, | Inhibits translation (protein |
| | | | Gram-negative bacteria | synthesis) |
| | | | not enterics, | |
| | | | Neisseria, Legionella, | |
| | | | Mycoplasma | |
| Polypeptides | Polymyxin | Bacillus polymyxa | Gram-negative bacteria | Damages cytoplasmic membranes |
| | Bacitracin | Bacillus subtilis | Gram-positive bacteria | Inhibits steps in murein |
| | | | | (peptidoglycan) biosynthesis and |
| | | | | assembly |
| Polyenes | Amphotericin | Streptomyces nodosus | Fungi | Inactivate membranes containing |
| | | | | sterols |
| | Nystatin | Streptomyces noursei | Fungi (Candida) | Inactivate membranes containing |
| | | | | sterols |
| Rifamycins | Rifampicin | Streptomyces mediterranei | Gram-positive and Gram- | Inhibits transcription (eubacterial |
| | | | negative bacteria, | RNA polymerase) |
| | | | Mycobacterium | |
| | | | tuberculosis | |
| Tetracyclines | Tetracycline | Streptomyces species | Gram-positive and Gram- | Inhibit translation (protein synthesis) |
| | | | negative bacteria, | |
| | | | Rickettsias | |
| Semisynthetic | Doxycycline | | Gram-positive and Gram- | Inhibit translation (protein synthesis) |
| tetracycline | | | negative bacteria, | |
| | | | Rickettsias Ehrlichia, | |
| | | | Borellia | |
| Chloramphenicol | Chloramphenicol | Streptomyces venezuelae | Gram-positive and Gram- | Inhibits translation (protein |
| | | | negative bacteria | synthesis) |

A. Antimicrobial Agents Used in the Treatment of Infectious Disease

An important property of a clinically-useful antimicrobial agent, especially from the patient's point of view, is its selective toxicity, i.e., that the agent acts in some way that inhibits or kills bacterial pathogens but has little or no toxic effect on the animal taking the drug. This implies that the biochemical processes in the bacteria are in some way different from those in the animal cells, and that the advantage of this difference can be taken in chemotherapy. Antibiotics may have a cidal (killing) effect or a static (inhibitory) effect on a range of microbes. The range of bacteria or other microorganisms that are affected by a certain antibiotic are is expressed as its spectrum of action. Antibiotics effective against prokaryotes which kill or inhibit a wide range of Gram-positive and Gram-negative bacteria are said to be broad spectrum. If effective mainly against Gram-positive or Gram-negative bacteria, they are narrow spectrum. If effective against a single organism or disease, they are referred to as limited spectrum. In accordance with the invention, certain adverse effects of antibiotics may be minimized by use of en ectophosphatase inhibitor in combination with the antibiotic, thereby allowing use of lower doses of antibiotics.

B. Kinds of Antimicrobial Agents and Their Primary Modes of Action

In one embodiment of the invention, ectophosphatase inhibitors are used in conjunction with cell wall synthesis inhibitors. Cell wall synthesis inhibitors generally inhibit some step in the synthesis of bacterial peptidoglycan. Generally they exert their selective toxicity against eubacteria because human cells lack cell walls.

Beta lactam antibiotics contain a 4-membered beta lactam ring. They are the products of two groups of fungi, Penicillium and Cephalosporium molds, and are correspondingly represented by the penicillins and cephalosporins. The beta lactam antibiotics inhibit the last step in peptidoglycan synthesis, the final cross-linking between peptide side chains, mediated by bacterial carboxypeptidase and transpeptidase enzymes. Beta lactam antibiotics are normally bactericidal and require that cells be actively growing in order to exert their toxicity.

Natural penicillins, such as Penicillin G or Penicillin V, are produced by fermentation of Penicillium chrysogenum. They are effective against streptococcus, gonococcus and staphylococcus, except where resistance has developed. They are considered narrow spectrum since they are not effective against Gram-negative rods.

Semisynthetic penicillins date to 1959. Mold produces a main part of the penicillin molecule (6-aminopenicillanic acid) which can be modified chemically by the addition of side chains. Many of these compounds have been developed to have distinct benefits or advantages over penicillin G, such as increased spectrum of activity (effectiveness against Gram-negative rods), resistance to penicillinase, effectiveness when administered orally, etc. Amoxycillin and Ampicillin have broadened spectra against Gram-negatives and are effective orally; Methicillin is penicillinase-resistant.

Clavulanic acid is a chemical sometimes added to a semisynthetic penicillin preparation. Thus, amoxycillin plus clavulanate is clavamox or augmentin. The clavulanate is not an antimicrobial agent. It inhibits beta lactamase enzymes and has given extended life to penicillinase-sensitive beta lactams.

Although nontoxic, penicillins occasionally cause death when administered to persons who are allergic to them. In the U.S. there are 300-500 deaths annually due to penicillin allergy. In allergic individuals the beta lactam molecule attaches to a serum protein which initiates an IgE-mediated inflammatory response.

Cephalolsporins are beta lactam antibiotics with a similar mode of action to penicillins that are produced by species of Cephalosporium. The have a low toxicity and a somewhat broader spectrum than natural penicillins. They are often used as penicillin substitutes, against Gram-negative bacteria, and in surgical prophylaxis. They are subject to degradation by some bacterial beta-lactamases, but they tend to be resistant to beta-lactamases from S. aureus.

Bacitracin is a polypeptide antibiotic produced by Bacillus species. It prevents cell wall growth by inhibiting the release of the muropeptide subunits of peptidoglycan from the lipid carrier molecule that carries the subunit to the outside of the membrane Teichoic acid synthesis, which requires the same carrier, is also inhibited. Bacitracin has a high toxicity which precludes its systemic use. It is present in many topical antibiotic preparations, and since it is not absorbed by the gut, it is given to “sterilize” the bowel prior to surgery.

Cell membrane inhibitors disorganize the structure or inhibit the function of bacterial membranes. The integrity of the cytoplasmic and outer membranes is vital to bacteria, and compounds that disorganize the membranes rapidly kill the cells. However, due to the similarities in phospholipids in eubacterial and eukaryotic membranes, this action is rarely specific enough to permit these compounds to be used systemically. The only antibacterial antibiotic of clinical importance that acts by this mechanism is Polymyxin, produced by Bacillus polymyxis. Polymyxin is effective mainly against Gram-negative bacteria and is usually limited to topical usage. Polymyxins bind to membrane phospholipids and thereby interfere with membrane function. Polymyxin is occasionally given for urinary tract infections caused by Pseudomonas that are gentamicin, carbenicillin and tobramycin resistant. The balance between effectiveness and damage to the kidney and other organs is dangerously close, and the drug should only be given under close supervision in the hospital.

In accordance with the invention, ectophosphatase inhibitors may also be contemporaneously administered and formulated in compositions with antibiotics that are protein synthesis inhibitors. Many therapeutically useful antibiotics inhibit some step in the complex process of translation. The attack is at one of the events occurring on the ribosome, rather than the stage of amino acid activation or attachment to a particular tRNA. Most have an affinity or specificity for 70S (as opposed to 80S) ribosomes, and they achieve their selective toxicity in this manner. The most important antibiotics with this mode of action are the tetracyclines, chloramphenicol, the macrolides (e.g. erythromycin) and the aminoglycosides (e.g. streptomycin).

The aminoglycosides are products of Streptomyces species and are represented by streptomycin, kanamycin, tobramycin and gentamicin. These antibiotics exert their activity by binding to bacterial ribosomes and preventing the initiation of protein synthesis. Aminoglycosides have been used against a wide variety of bacterial infections caused by Gram-positive and Gram-negative bacteria. Streptomycin has been used extensively as a primary drug in the treatment of tuberculosis. Gentamicin is active against many strains of Gram-positive and Gram-negative bacteria, including some strains of Pseudomonas aeruginosa. Kanamycin (a complex of three antibiotics, A, B and C) is active at low concentrations against many Gram-positive bacteria, including penicillin-resistant staphylococci. Gentamicin and Tobramycin are mainstays for treatment of Pseudomonas infections. An unfortunate side effect of aminoglycosides has tended to restrict their usage: prolonged use is known to impair kidney function and cause damage to the auditory nerves leading to deafness.

The tetracyclines consist of eight related antibiotics which are all natural products of Streptomyces, although some can now be produced semisynthetically. Tetracycline, chlortetracycline and doxycycline are the best known. The tetracyclines are broad-spectrum antibiotics with a wide range of activity against both Gram-positive and Gram-negative bacteria. The tetracyclines act by blocking the binding of aminoacyl tRNA to the A site on the ribosome. Tetracyclines inhibit protein synthesis on isolated 70S or 80S (eukaryotic) ribosomes, and in both cases, their effect is on the small ribosomal subunit. However, most bacteria possess an active transport system for tetracycline that will allow intracellular accumulation of the antibiotic at concentrations 50 times as great as that in the medium. This greatly enhances its antibacterial effectiveness and accounts for its specificity of action, since an effective concentration cannot be accumulated in animal cells. Thus a blood level of tetracycline which is harmless

The tetracyclines have a remarkably low toxicity and minimal side effects when taken by animals. The combination of their broad spectrum and low toxicity has led to their overuse and misuse by the medical community and the wide-spread development of resistance has reduced their effectiveness. Nonetheless, tetracyclines still have some important uses, such as in the treatment of Lyme disease.

Chloramphenicol has a broad spectrum of activity but it exerts a bacteriostatic effect. It is effective against intracellular parasites such as the rickettsiae. Unfortunately, aplastic anemia, which is dose related develops in a small proportion (1/50,000) of patients. Chloramphenicol was originally discovered and purified from the fermentation of a Streptomyces, but currently it is produced entirely by chemical synthesis. Chloramphenicol inhibits the bacterial enzyme peptidyl transferase thereby preventing the growth of the polypeptide chain during protein synthesis.

Chloramphenicol is entirely selective for 70S ribosomes and does not affect 80S ribosomes. Its unfortunate toxicity towards the small proportion of patients who receive it is in no way related to its effect on bacterial protein synthesis. However, since mitochondria probably originated from prokaryotic cells and have 70S ribosomes, they are subject to inhibition by some of the protein synthesis inhibitors including chloroamphenicol. This likely explains the toxicity of chloramphenicol. The eukaryotic cells most likely to be inhibited by chloramphenicol are those undergoing rapid multiplication, thereby rapidly synthesizing mitochondria. Such cells include the blood forming cells of the bone marrow, the inhibition of which could present as aplastic anemia. Chloramphenicol was once a highly prescribed antibiotic and a number of deaths from anemia occurred before its use was curtailed. Now it is seldom used in human medicine except in life-threatening situations (e.g. typhoid fever).

The Macrolides are a family of antibiotics whose structures contain large lactone rings linked through glycoside bonds with amino sugars. The most important members of the group are erythromycin and oleandomycin. Erythromycin is active against most Gram-positive bacteria, Neisseria, Legionella and Haemophilus, but not against the Enterobacteriaceae. Macrolides inhibit bacterial protein synthesis by binding to the 50S ribosomal subunit. Binding inhibits elongation of the protein by peptidyl transferase or prevents translocation of the ribosome or both. Macrolides are bacteriostatic for most bacteria but are cidal for a few Gram-positive bacteria.

In further embodiments of the invention, ectophosphatase inhibitors may be contemporaneously administered and formulated in compositions with antibiotics that affect nucleic acids, including chemotherapeutic agents that affect the synthesis of DNA or RNA, or can bind to DNA or RNA so that their messages cannot be read. In either case, this can block the growth of cells. The majority of these drugs are unselective, however, and affect animal cells and bacterial cells alike and therefore have no therapeutic application. Two nucleic acid synthesis inhibitors which have selective activity against prokaryotes and some medical utility are nalidixic acid and rifamycins.

Nalidixic acid is a synthetic chemotherapeutic agent which has activity mainly against Gram-negative bacteria. Nalidixic acid belongs to a group of compounds called quinolones. Nalidixic acid is a bactericidal agent that binds to the DNA gyrase enzyme (topoisomerase) which is essential for DNA replication and allows supercoils to be relaxed and reformed. Binding of the drug inhibits DNA gyrase activity.

Some quinolones penetrate macrophages and neutrophils better than most antibiotics and are thus useful in treatment of infections caused by intracellular parasites. However, the main use of nalidixic acid is in treatment of lower urinary tract infections (UTI). The compound is unusual in that it is effective against several types of Gram-negative bacteria such as E. coli, Enterobacter aerogentes, K. pneumoniae and Proteus species which are common causes of UTI. It is not usually effective against Pseudomonas aeruginosa, and Gram-positive bacteria are resistant.

The rifamycins are also the products of Streptomyces. Rifampicin is a semisynthetic derivative of rifamycin that is active against Gram-positive bacteria (including Mycobacterium tuberculosis) and some Gram-negative bacteria. Rifampicin acts quite specifically on eubacterial RNA polymerase and is inactive towards RNA polymerase from animal cells or towards DNA polymerase. The antibiotic binds to the beta subunit of the polymerase and apparently blocks the entry of the first nucleotide which is necessary to activate the polymerase, thereby blocking mRNA synthesis. It has been found to have greater bactericidal effect against M. tuberculosis than other anti-tuberculosis drugs, and it has largely replaced isoniazid as one of the front-line drugs used to treat the disease, especially when isoniazid resistance is indicated. It is effective orally and penetrates well into the cerebrospinal fluid and is therefore useful for treatment of tuberculosis meningitis and meningitis caused by Neisseria meningitidis.

In accordance with the invention, ectophosphatase inhibitors may also be administered with competitive inhibitors, including mostly all synthetic chemotherapeutic agents. Most are “growth factor analogs” which are structurally similar to a bacterial growth factor but which do not fulfill its metabolic function in the cell. Some are bacteriostatic and some are bactericidal.

Sulfonamides were originally introduced as chemotherapeutic agents, one of which, prontosil, had the effect of curing mice with infections caused by beta-hemolytic streptococci. Chemical modifications of the compound sulfanilamide gave compounds with even higher and broader antibacterial activity. The resulting sulfonamides have broadly similar antibacterial activity, but differ widely in their pharmacological actions. Bacteria which are almost always sensitive to the sulfonamides include Streptococcus pneumoniae, beta-hemolytic streptococci and E. coli. The sulfonamides have been extremely useful in the treatment of uncomplicated UTI caused by E. coli, and in the treatment of meningococcal meningitis, because they cross the blood-brain barrier.

The sulfonamides (e.g. Gantrisin) and Trimethoprim are inhibitors of the bacterial enzymes required for the synthesis of tetrahydofolic acid (THF), the vitamin form of folic acid essential for 1-carbon transfer reactions. Sulfonamides are structurally similar to para aminobenzoic acid (PABA), the substrate for the first enzyme in the THF pathway, and they competitively inhibit that step. Trimethoprim is structurally similar to dihydrofolate (DHF) and competitively inhibits the second step in THF synthesis mediated by the DHF reductase. Animal cells do not synthesize their own folic acid but obtain it in a preformed fashion as a vitamin. Since animals do not make folic acid, they are not affected by these drugs, which achieve their selective toxicity for bacteria on this basis.

Three additional synthetic chemotherapeutic agents have been used in the treatment of tuberculosis: isoniazid (INH), paraaminosalicylic acid (PAS), and ethambutol. The usual strategy in the treatment of tuberculosis has been to administer a single antibiotic (historically streptomycin, but now, most commonly, rifampicin is given) in conjunction with INH and ethambutol. Since the tubercle bacillus rapidly develops resistance to the antibiotic, ethambutol and INH are given to prevent outgrowth of a resistant strain. It must also be pointed out that the tubercle bacillus rapidly develops resistance to ethambutol and INH if either drug is used alone. Ethambutol inhibits incorporation of mycolic acids into the mycobacterial cell wall. Isoniazid has been reported to inhibit mycolic acid synthesis in mycobacteria and since it is an analog of pyridoxine (Vitamin B6) it may inhibit pyridoxine catalyzed reactions as well. Isoniazid is activated by a mycobacterial peroxidase enzyme and destroys several targets in the cell. PAS is an anti-folate. PAS was once a primary anti-tuberculosis drug, but now it is a secondary agent, having been largely replaced by ethambutol.

C. Inhibition of Drug Resistance in Microorganisms to Treat Infection

The present invention also relates to methods for inhibiting or ameliorating infection in animals and humans caused by microorganisms. For example, treatment of bacterial and fungal infections may be augmented or effected using inhibitory mechanisms against an ectophosphatase to modify the ATP gradient across biological membranes. The invention is useful in the inhibition or amelioration of a wide range of infections including, but not limited to, gram negative bacterial infection including gram-negative sepsis, gram-negative endotoxin-related hypotension and shock, rabies, cholera, tetanus, lymes disease, tuberculosis, Candida albicans, Chlamydia, etc.

The inhibition or amelioration of the infections may involve the administration of an anti-microbial agent (such as an antibiotic or an antifungal agent) with the concurrent administration of the aforementioned compositions. Additionally, inhibitors of ectophosphatases or ABC transporters may be administered via a physiologically acceptable carrier as described above. In one embodiment of the invention, the inhibitor of ectophosphatase is selected from the compounds represented by formulas I-XX.

Certain aspects of the current invention thus concern the inhibition of cell growth by contacting a cell with an inhibitor of an ectophosphatase with contemporaneous administration of other agents capable of inhibiting cell growth or killing the cell. In this manner, therapeutic benefit may be obtained for the treatment of bacterial infections. Examples of types of bacteria that could be inhibited and bacterial infections that could potentially be treated or prevented with the invention, include, but are not limited to, the 83 or more distinct serotypes of pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, Staphylococci, such as Staphylococcus epidermidis, Staphylococcus aureus, particularly in the nasopharynx, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Neisseria meningitidis, Neisseria gonorrhoeae, Moraxella catarrhalis, Corynebacterium diphtheriae, Corynebacterium ulceratis, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species and related organisms. The invention may also find use, for example, against gram negative bacteria such as Klebsiella pneumoniae, Escherichia coli, Proteus, Serratia species, Acinetobacter, Yersinia pestis, Francisella tularensis, Enterobacter species, Bacteriodes and Legionella species and the like.

### IV. Chemotherapeutics

A variety of chemotherapeutic agents are suitable for use with the invention and are known to those of skill in the art. In accordance with the invention, therapeutic benefit may be obtained by contemporaneous administration of one or more ectophosphatase inhibitor and one or more such chemotherapeutic agent(s). Co-administration of chemotherapeutics with ectophosphatase inhibitors in accordance with the invention may be used to increase therapeutic effectiveness of a chemotherapeutic and may allow use of lowered doses. Such techniques may further allow treatment of chemotherapy-resistant tumor cells.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating agents may be used with an ectophosphatase inhibitor. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Further agents for use with ectophosphatase inhibitors in accordance with the invention include, for example, compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors also may be used with ectophosphatase inhibitors. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU is applicable in a wide range of carriers, including topical, with intravenous administration in doses ranging from 3 to 15 mg/kg/day being commonly used.

Exemplary and non-limiting chemotherapeutic agents for use in combination with an ectophosphatase inhibitor in accordance with the invention are listed in Table 3. Accordingly, compositions comprising these agents and one or more ectophosphatase inhibitor(s) form one part of the invention, as do methods for the administration thereof. Each of the agents listed below are exemplary and by no means limiting. In this regard, the skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Specifically contemplated by the inventors are compositions comprising and ectophosphatase inhibitor and a chemotherapeutic agent set forth in Table 3, as well as methods comprising the use thereof

TABLE 3
Chemotherapeutic Agents Useful In Neoplastic Disease

| | | NONPROPRIETARY | |
|---|---|---|---|
| | TYPE OF | NAMES | |
| CLASS | AGENT | (OTHER NAMES) | DISEASE |
| | | Mechlorethamine (HN₂) | Hodgkin's disease, |
| | | | non-Hodgkin's lymphomas |
| | Nitrogen | Cyclophosphamide | Acute and chronic |
| | Mustards | Ifosfamide | lymphocytic leukemias, |
| | | | Hodgkin's disease, |
| | | | non-Hodgkin's lymphomas, |
| | | | multiple myeloma, |
| | | | neuroblastoma, breast, ovary, |
| | | | lung, Wilms' tumor, cervix, |
| | | | testis, soft-tissue sarcomas |
| | | Melphalan (L-sarcolysin) | Multiple myeloma, breast, |
| | | | ovary |
| | | Chlorambucil | Chronic lymphocytic |
| | | | leukemia, primary |
| | | | macroglobulinemia, |
| | | | Hodgkin's disease, |
| | | | non-Hodgkin's lymphomas |
| Alkylating | Ethylenimenes | Hexamethylmelamine | Ovary |
| Agents | and | | |
| | Methylmelamines | | |
| | | Thiotepa | Bladder, breast, ovary |
| | Alkyl | Busulfan | Chronic granulocytic leukemia |
| | Sulfonates | | |
| | | Carmustine (BCNU) | Hodgkin's disease, |
| | | | non-Hodgkin's lymphomas, |
| | | | primary brain tumors, multiple |
| | | | myeloma, malignant |
| | | | melanoma |
| | Nitrosoureas | Lomustine (CCNU) | Hodgkin's disease, |
| | | | non-Hodgkin's lymphomas, |
| | | | primary brain tumors, |
| | | | small-cell lung |
| | | Semustine | Primary brain tumors, |
| | | (methyl-CCNU) | stomach, colon |
| | | Streptozocin | Malignant pancreatic |
| | | (streptozotocin) | insulinoma, malignant |
| | | | carcinoid |
| | Triazines | Dacarbazine (DTIC; | Malignant melanoma, |
| | | dimethyltriazenoimidazolecarboxamide) | Hodgkin's disease, soft-tissue |
| | | | sarcomas |
| Antimetabolites | Folic Acid | Methotrexate | Acute lymphocytic leukemia, |
| | Analogs | (amethopterin) | choriocarcinoma, mycosis |
| | | | fungoides, breast, head and |
| | | | neck, lung, osteogenic |
| | | | sarcoma |
| | Pyrimidine | Fluouracil | Breast, colon, stomach, |
| | Analogs | (5-fluorouracil; 5-FU) | pancreas, ovary, head and |
| | | Floxuridine | neck, urinary bladder, |
| | | (fluorode-oxyuridine; | premalignant skin lesions |
| | | FUdR) | (topical) |
| Antimetabolites, | | Cytarabine (cytosine | Acute granulocytic and acute |
| continued | | arabinoside) | lymphocytic leukemias |
| | | Mercaptopurine | Acute lymphocytic, acute |
| | | (6-mercaptopurine; | granulocytic and chronic |
| | | 6-MP) | granulocytic leukemias |
| | Purine Analogs | Thioguanine | Acute granulocytic, acute |
| | and Related | (6-thioguanine; TG) | lymphocytic and chronic |
| | Inhibitors | | granulocytic leukemias |
| | | Pentostatin | Hairy cell leukemia, mycosis |
| | | (2-deoxycoformycin) | fungoides, chronic |
| | | | lymphocytic leukemia |
| | | Vinblastine (VLB) | Hodgkin's disease, |
| | | | non-Hodgkin's lymphomas, |
| | | | breast, testis |
| | Vinca Alkaloids | Vincristine | Acute lymphocytic leukemia, |
| | | | neuroblastoma, Wilms' tumor, |
| | | | rhabdomyosarcoma, |
| | | | Hodgkin's disease, |
| | | | non-Hodgkin's lymphomas, |
| | | | small-cell lung |
| | Epipodophyllotoxins | Etoposide | Testis, small-cell lung and |
| | | Tertiposide | other lung, breast, Hodgkin's |
| | | | disease, non-Hodgkin's |
| | | | lymphomas, acute |
| | | | granulocytic leukemia, |
| | | | Kaposi's sarcoma |
| Natural | | Dactinomycin | Choriocarcinoma, Wilms' |
| Products | | (actinomycin D) | tumor, rhabdomyosarcoma, |
| | | | testis, Kaposi's sarcoma |
| | | Daunorubicin | Acute granulocytic and acute |
| | | (daunomycin; | lymphocytic leukemias |
| | | rubidomycin) | |
| | Antibiotics | Doxorubicin | Soft-tissue, osteogenic and |
| | | | other sarcomas; Hodgkin's |
| | | | disease, non-Hodgkin's |
| | | | lymphomas, acute leukemias, |
| | | | breast, genitourinary, thyroid, |
| | | | lung, stomach, neuroblastoma |
| | | Bleomycin | Testis, head and neck, skin, |
| | | | esophagus, lung and |
| | | | genitourinary tract; Hodgkin's |
| | | | disease, non-Hodgkin's |
| | | | lymphomas |
| | Antibiotics, | Plicamycin | Testis, malignant |
| | continued | (mithramycin) | hypercalcemia |
| Natural | | Mitomycin (mitomycin | Stomach, cervix, colon, breast, |
| Products, | | C) | pancreas, bladder, head and |
| continued | | | neck |
| | Enzymes | L-Asparaginase | Acute lymphocytic leukemia |
| | Biological | Interferon alfa | Hairy cell leukemia., Kaposi's |
| | Response | | sarcoma, melanoma, |
| | Modifiers | | carcinoid, renal cell, ovary, |
| | | | bladder, non-Hodgkin's |
| | | | lymphomas, mycosis |
| | | | fungoides, multiple myeloma, |
| | | | chronic granulocytic leukemia |
| | Platinum | Cisplatin (cis-DDP) | Testis, ovary, bladder, head |
| | Coordination | Carboplatin | and neck, lung, thyroid, |
| | Complexes | | cervix, endometrium, |
| | | | neuroblastoma, osteogenic |
| | | | sarcoma |
| | Anthracenedione | Mitoxantrone | Acute granulocytic leukemia, |
| | | | breast |
| Miscellaneous | Substituted Urea | Hydroxyurea | Chronic granulocytic |
| Agents | | | leukemia, polycythemia vera, |
| | | | essental thrombocytosis, |
| | | | malignant melanoma |
| | Methyl | Procarbazine | Hodgkin's disease |
| | Hydrazine | (N-methylhydrazine, | |
| | Derivative | MIH) | |
| | Adrenocortical | Mitotane (o, p′-DDD) | Adrenal cortex |
| | Suppressant | Aminoglutethimide | Breast |
| | Adrenocorticosteroids | Prednisone (several other | Acute and chronic |
| | | equivalent preparations | lymphocytic leukemias, |
| | | available) | non-Hodgkin's lymphomas, |
| | | | Hodgkin's disease, breast |
| Hormones and | Progestins | Hydroxyprogesterone | Endometrium, breast |
| Antagonists | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations available) | |
| | Antiestrogen | Tamoxifen | Breast |
| | Androgens | Testosterone propionate | Breast |
| | | Fluoxymesterone (other | |
| | | preparations available) | |
| | Antiandrogen | Flutamide | Prostate |
| | Gonadotropin-releasing | Leuprolide | Prostate |
| | hormone | | |
| | analog | | |

Administration of compositions provided by the invention may be local or systemic, using a suitable physiological carrier. Other compounds which aid in the uptake or stability of these agents, or which have beneficial activity, may also be included in the formulations of the invention. Certain embodiments of the invention thus provide pharmaceutical composition comprising an ectophosphatase inhibitor. The composition may further comprise, in certain embodiments, a cytotoxic agent, including a chemotherapeutic agent set forth herein. For example, by contacting a tumor cell either alone with the ectophosphatase inhibitor or in combination with one or more chemotherapeutic agents, therapeutic benefit may be obtained. Particular benefit may be obtained where a tumor cell is resistant to at least one chemotherapeutic agent. This may enhance the overall anti-tumor activity achieved by therapy, and/or may be used to prevent or combat multi-drug tumor resistance.

### V. Assays and Methods for Screening Active Compounds

In one embodiment of the invention, assays are provided for screening compositions comprising combinations of ectophosphatase inhibitors and a selected cytotoxic agent, for example, a herbicide, fungicide, antibiotic, insecticide or chemotherapeutic agent. A number of assay formats are known to those of skill in the art and may be used in this regard. These include assays of biological activities as well as assays of chemical properties. The results of these assays provide important inferences as to the properties of compounds as well as their potential applications in treating human or other mammalian patients for infection and hyperproliferative diseases as well as in various agricultural applications. Assays deemed to be of particular utility in this regard include in vivo and in vitro screens of activity and immunoassays.

(i) Screening for Herbicidal Activity

One aspect of the current invention provides herbicidal compositions having improved herbicidal activity. In accordance with the invention, assays for herbicidal activity may be used to asses the relative efficacy of combinations of ectophosphatase inhibitors and herbicidal agents. Numerous assays formats for analyzing herbicidal activity are known to those of skill in the art and may be used in this regard. A straightforward means for testing activity comprises the serial application of test compositions to plants and/or plant parts, for example, by leaf painting. Plants are otherwise grown under comparable conditions, followed by serial applications of test and control compositions are applied. Control treatments may be used comprising herbicide compositions lacking ectophosphatase inhibitors, thereby identifying the presence of absence of activity as a syngerist. Herbicidal activity is then measured by analysis of the effect of the composition on plant viability.

Commercial herbicide formulations are well known to those of skill in the art and will generally be used in assays. However, it will typically be preferred to dilute herbicidal compositions and/or administer reduced amounts of the compositions such that the relative herbicidal activity of a composition may be quantified. Reductions in the lethality of herbicidal compositions allows the identification of effectiveness between herbicidal compositions. Thus, in particular embodiments of the invention, test and control compositions are administered in rates and/or amounts of from about 90% to about 5% of the commercial application rate for a given herbicide.

Following application of herbicidal test compositions, herbicidal activity and effectiveness is measured. Through comparisons with applications of control compositions, the effectiveness of the compositions may be determined.

Tests may be carried out on whole plants as well as plant parts or cell. For example, plant cells in tissue culture may be screened for herbicidal activity. The preparation of plant tissue-cultures is well known to those of skill in the art.

(ii) In Vivo Assays

The present invention encompasses the use of various animal models. Here, the identity seen between human and mouse provides an excellent opportunity to examine the function of a potential therapeutic agent, for example, an antiinfective or chemotherapeutic administered in combination with an ectophosphatase inhibitor. In the case of chemotherapeutics, one can utilize cancer models in mice that will be highly predictive of cancers in humans and other mammals. These models may employ the orthotopic or systemic administration of tumor cells to mimic primary and/or metastatic cancers. Alternatively, one may induce cancers in animals by providing agents known to be responsible for certain events associated with malignant transformation and/or tumor progression. Similarly, numerous animal models are available for analysis of antinfectives, including insecticides, antibiotics and antifungal agents. In this manner, all that is commonly required is infection of the relevant animal with a target infective agent and analysis of therapeutic benefit relative to prior antiinfective compositions.

Treatment of animals with test compositions will involve the administration of the composition, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake. Through comparisons of active agents alone and active agents in combination with ectophosphatase inhibitors, synergistic combinations may be readily confirmed.

(iii) Confirmatory In Vivo and Clinical Studies

It will be understood by those of skill in the art that therapeutic compositions should generally be tested in an in vivo setting prior to use in a human subject. Such pre-clinical testing in animals is routine in the art. To conduct such confirmatory tests, all that is required is an art-accepted animal model of the disease in question, such as an animal bearing a solid tumor or infective agent. Any animal may be used in such a context, such as, e.g., a mouse, rat, guinea pig, hamster, rabbit, dog, chimpanzee, or such like. Studies using small animals such as mice are widely accepted as being predictive of clinical efficacy in humans, and such animal models may thus find use in the context of the present invention as they are readily available and relatively inexpensive, at least in comparison to other experimental animals.

The manner of conducting an experimental animal test will be straightforward to those of ordinary skill in the art. All that is required to conduct such a test is to establish equivalent treatment groups, and to administer the test compounds to one group while various control studies are conducted in parallel on the equivalent animals in the remaining group or groups. One monitors the animals during the course of the study and, ultimately, one sacrifices the animals to analyze the effects of the treatment.

In the context of the treatment of tumors, it is contemplated that effective amounts of chemotherapeutic compositions will be those that generally result in at least about 10% of the cells within a tumor exhibiting cell death or apoptosis. Preferably, at least about 20%, about 30%, about 40%, or about 50%, of the cells at a particular tumor site will be killed. Most preferably, 100% of the cells at a tumor site will be killed.

The extent of cell death in a tumor is assessed relative to the maintenance of healthy tissues in all of the areas of the body. It will be preferable to use doses of chemotherapeutic compositions capable of inducing at least about 60%, about 70%, about 80%, about 85%, about 90%, about 95% up to and including 100% tumor necrosis, so long as the doses used do not result in significant side effects or other untoward reactions in the animal. All such determinations can be readily made and properly assessed by those of ordinary skill in the art. For example, attendants, scientists and physicians can utilize such data from experimental animals in the optimization of appropriate doses for human treatment. In subjects with advanced disease, a certain degree of side effects can be tolerated. However, patients in the early stages of disease can be treated with more moderate doses in order to obtain a significant therapeutic effect in the absence of side effects. The effects observed in such experimental animal studies should preferably be statistically significant over the control levels and should be reproducible from study to study.

Those of ordinary skill in the art will further understand that combinations and doses of the compositions provided by the invention that result in tumor-specific necrosis towards the lower end of the effective ranges may nonetheless still be useful in connection with the present invention. For example, in embodiments where a continued application of the active agents is contemplated, an initial dose that results in only about 10% necrosis will nonetheless be useful, particularly as it is often observed that this initial reduction “primes” the tumor to further destructive assault upon subsequent re-application of the therapy. In any event, even if upwards of about 40% or so tumor inhibition is not ultimately achieved, it will be understood that any induction, of thrombosis and necrosis is nonetheless useful in that it represents an advance over the state of the patients prior to treatments. Still further, it is contemplated that a dose which prevents or decreases the likelihood of either metastasis or de novo carcinogenesis would also be of therapeutic benefit to a patient receiving the treatment.

As discussed above in connection with the in vitro test system, it will naturally be understood that combinations of agents intended for use together should be tested and optimized together. The compositions of the invention can be straightforwardly analyzed in the combinations set forth herein. Analysis of the combined effects of such agents would be determined and assessed according to the guidelines set forth above.

(iv) In Vitro Assays

In one embodiment of the invention, screening of a composition provided by the invention is conducted in vitro to identify those compounds capable of synergizing therapeutic agents, including cytotoxic agents such as antibiotics, fungicides and chemotherapeutic agents, as well as other types of biologically-active agents. In the case of killing of tumor cells, cytotoxicity is generally exhibited by necrosis or apoptosis. Necrosis is a relatively common pathway triggered by external signals. During this process, the integrity of the cellular membrane and cellular compartments is lost. On the other hand, apoptosis, or programmed cell death, is a highly organized process of morphological events that is synchronized by the activation and deactivation of specific genes (Thompson et al., 1992; Wyllie, 1985). In the case of pesticides, the selective cytotoxic action against the infective agent, including an insect, bacterial or fungal pathogen, is typically analyzed.

An efficacious means for in vitro assaying of cytoxicity comprises the systematic exposure of a panel of cells to selected compositions. For example, in the case of cancer, many tumor cell lines are available for implementing assays, including human ovarian, leukemic, breast, prostate, melanoma and renal cancer cells.

In vitro determinations of the efficacy of a compound in killing tumor cells may be achieved, for example, by assays of the expression and induction of various genes involved in cell-cycle arrest (p21, p27; inhibitors of cyclin dependent kinases) and apoptosis (bcl-2, bcl-x_{L} and bax). To carry out this assay, cells are treated with the test compound, lysed, the proteins isolated, and then resolved on SDS-PAGE gels and the gel-bound proteins transferred to nitrocellulose membranes. The membranes are first probed with the primary antibodies (e.g., antibodies to p21, p27, bax, bcl-2 and bcl-x₁, etc.) and then detected with diluted horseradish peroxidase conjugated secondary antibodies, and the membrane exposed to ECL detection reagent followed by visualization on ECL-photographic film. Through analysis of the relative proportion of the proteins, estimates may be made regarding the percent of cells in a given stage, for example, the G0/G1 phase, S phase or G2/M phase.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Inhibition of Antibiotic Resistant Bacteria with Ectophosphatase Inhibitors

A. Summary of Protocol and Results

The purpose of this study was to evaluate various ectophosphatase inhibitor compounds for any potentiating effect or resistance reversal with methicillin resistant Staphylococcus aureus (MRSA). Two strains of Staphylococcus aureus were obtained from the American Type Culture Collection (ATCC). Those obtained were a previously characterized methicillin resistant strain (# 43300) and a previously characterized methicillin sensitive strain (# 29213) to serve as a control. Strains were received as lyophilized pellets and were rehydrated according to the ATCC Product Information Sheet using Trypticase Soy Broth.

Mueller-Hinton agar plates were prepared containing 1 of 6 inhibitor compounds investigated. The compounds were dissolved in DMSO at concentrations of 20 mg/ml. 25 μl of this stock solution was added to 25 mls of media just prior to cooling to solidification to produce plates containing 20 μg/ml of inhibitor compound. Control plates containing 25 μl DMSO only were also prepared in the same fashion.

For each of the two strains, 5 isolated colonies were picked from overnight culture plates and used to inoculate a 0.85% NaCl solution. The turbidity of each culture was adjusted using 0.85% NaCl until the turbidity of each matched a 0.5 McFarland turbidity standard. Each strain was swabbed onto control plates. The MRSA strain was swabbed also onto the inhibitor plates. Plates were allowed to dry for 5 minutes. A disk containing 5 μg of methicillin (obtained for the purpose of susceptibility testing from Bioanalyse Co., Ltd.) was placed onto the surface of all plates. Plates were incubated at 37° C.

Plates were read after 24 hours. It was noticed at this time that one plate, containing inhibitor compound NGXT1914 (Formula XV), was clear—there was no bacterial growth at all. All other plates had confluent growth except at the zone of inhibition of the methicillin disk.

Protocols for the studies were according to NCCLS guidelines (M2-A7, M100-S11) for antimicrobial susceptibility testing. Methodology for the serum bactericidal test was according to (Tentative Guideline) NCCLS M21-T, 1992. Methods for determining bactericidal activity of antimicrobial agents was according to (Tentative Guideline) NCCLS M26-T.1992. Methods for dilution antimicrobial susceptibility tests for bacteria that grow aerobically. 3rd ed. NCCLS M7-A3.1993. Protocols for evaluating dehydrated Mueller-Hinton agar were according to NCCLS M6-A, 1996. Performance standards for antimicrobial susceptibility testing were according to NCCLS M100-S8, 1998. The screening test for Oxacillin-resistant Staphylococci was according to NCCLS M7-A4. Performance standards for antimicrobial disk and dilution susceptibility tests for bacteria isolated from animals was according to (Approved Standard) NCCLS M31-A. Performance standards for antimicrobial susceptibility testing were according to NCCLS M100-S8.1998. Screening test for Oxacillin-resistant Staphylococci was according to NCCLS M7-A4 Susceptibility test panels.

B. Confirmation of Results

In order to ascertain whether the result with inhibitor NGXT1914 was artifactual, both MRSA and MSSA were tested on Mueller-Hinton agar plates containing varying concentrations of NGXT1914. First, plates were prepared as above with the following concentrations of inhibitor compound: 0 μg/ml (DMSO controls), 1 μg/ml, 5 μg/ml, 10 μg/ml, and 25 μg/ml. Inhibitor concentrations were prepared such that all plates contained 25 μl total DMSO. For each of the two strains, 5 isolated colonies were picked from fresh overnight culture plates and used to inoculate a 0.85% NaCl solution. The turbidity of each culture was adjusted using 0.85% NaCl until the turbidity of each matched a 0.5 McFarland turbidity standard. Both strains were each swabbed onto both control and inhibitor plates. Plates were allowed to dry for 5 minutes and were placed into the incubator. Plates were read after 24 hours (Dec. 7, 2001). The results were as follows:

| Concentration | |
|---|---|
| (μg/ml NGXT1914) | Result |
| 25 μg/ml | No growth at all for either strain |
| 10 μg/ml | No growth at all for MRSA strain. Some |
| | small isolated colonies on the MSSA plate. |
| 5 μg/ml | Only a few small colonies on the MRSA |
| | plate. Confluent growth on the MSSA |
| | plate. |
| 1 μg/ml | Confluent growth on both plates. |
| Controls | Confluent growth on both plates. |

C. Conclusions

Inhibitor NGXT 1914 inhibits growth of both resistant and sensitive Staph strains, however it is more effective against the methicillin resistant strain. This compound was tested with other cell types (mammalian cell lines, plants (Arabidopsis thaliana) yeast, Pseudomonas aeruginosa) and has not inhibited growth of any cell line or organism at the concentrations tested. Since NGXT1914 is an ectophosphatase inhibitor, it is possible that Staph, particularly the MRSA, is dependent on a phosphatase that is an ideal substrate for NGXT1914. The results obtained indicated that NGXT1914 was more effective against MRSA versus MSSA than previously identified compounds.

### Example 2

### Inhibition of Chemotherapy-Resistant Tumor Cells with an Ectophosphatase Inhibitor

In order to determine the effect of ectophosphatase inhibitors on tumor cells resistant to a chemotherapeutic agent, 2 breast cancer tumor cell lines were tested, a vinblastine resistant line (SW-13Vb003) and its vinblastine sensitive parent line, SW-13. A standard 4 day incubation at 37° C. and 5% CO₂ was used. IC₅₀ tests (NTT) were done using standard methodology in 96 well plate format with inhibitor concentrations ranging from 0-90 μg/ml (DMEM media). Results showed that 3 of the inhibitor compounds tested, NGXT194 (Formula VI), NGXT196 (Formula VIII), and NGXT1915 (Formula X), produced lower IC₅₀ values with the resistant cell line than with the sensitive line. For NGXT194, SW-13 was sensitive at 20 μg/ml, whereas the vinblastine resistant line SW-13Vb003 was sensitive at 10 μg/ml. For NGXT196, SW-13 was sensitive at 30 μg/ml, whereas the vinblastine resistant line SW-13Vb003 was sensitive at 10 μg/ml. For NGXT1915, SW-13 was sensitive at 50 μg/ml, whereas the vinblastine resistant line SW-13Vb003 was sensitive at 20 μg/ml.

Based on the inventors previous findings concerning the role of ectophosphatases in drug resistance, it seems likely that many resistant cells have upregulated phosphatases and that inhibiting a phosphatase critical to maintaining these cells could result in cell death. One hypothesis is that once these phosphatase enzymes are upregulated or are used in drug resistance their function becomes one that is vital to the cell. Since this phenomenon has been seen now in a bacterial resistance model it might be expected that the ectophosphatase inhibiting compounds could preferentially kill other types of resistant cells or act as biocides to certain cells.

### Example 3

### Over-Expression of Ectophosphatase does not Increase the Cellular Uptake of Adenosine

A. Materials and Methods

Transgenic Plant Construction: psNTP9 (Pisum Sativum apyrase, GenBank accession #Z32743) was subcloned as a Sall to Xbal fragment into pKYLX71 (Schardl et al, 1987, supra.). This plasmid was transformed into A. tumefaciens GV3101 [pMP90] pKYLX71 (Koncz and Shell, 1986), which was used to infect root call from Ws ecotype Arabidopsis thaliana under kanamycin selection (Valvekens et al., 1992). Four individual lines, obtained from separate calli, were propagated to the third generation (T3).

Subcellular Apyrase Distribution in Pea: Etiolated pea plumules served as the tissue source for nuclei and cytoplasm isolation as described by Chen and Roux (Plant Physiol. 81:609-612 (1986)). Plasma membrane was prepared from 30 g of pea root tissue (Zhu Mei Jun and Chen Jia; 1995, Acta Botanica Sinica 37:942-949). Western analysis was performed on 15-30 pg of protein from cytoplasm, plasma membrane and nuclei using a polyclonal anti-apyrase antibody raised against the purified pea protein (Tong et al., 1993). To determine the orientation of the pea apyrase in the pea plasma membrane, outside-out vesicles were prepared and the accessibility of the enzyme was determined by selective trypsin proteolysis, or membrane shaving, followed by activity assays and western blotting.

Phosphate uptake experiments and growth assays: In all experiments the growth media did not contain sugar, and plants were grown in sterile culture at 22° C. under 150-200 pE of continuous light. Unless otherwise noted, a standard 0.8% agar medium (Becton Dickenson, Cockeysville, Md.) containing 100 μM phosphate was used for uptake assays (Somerville et al., 1982). Plants used for the phosphate uptake experiments were grown singly in 1 ml of the standard agar medium for 15 days prior to the experiment. On the day of the experiment, 10 μCi³²P was applied to the side of the culture dish and allowed to diffuse through the agar. The lids of 95 mm×15 mm tissue' culture dishes (Fisher, Pittsburgh, Pa.) were removed to facilitate transpiration.

After 18 hours, the plants were removed from the medium. The aerial portions of the plant not in contact with the agar were weighed and counted by liquid scintillation. For each plant the entire root system was carefully pulled from the agar and washed in ice cold water prior to scintillation counting. To measure the transport of the products of ATP hydrolysis by the transgenic plants overexpressing apyrase and by wild-type plants, [2,8³H]ATP, [α³²P]ATP, and [γ³²P]ATP (Amersham) were fed to 15-day-old plants in separate treatments. All treatments were analyzed for significance in a T-test (n>46 for all groups, *P<0.05, error bars=s.e.m.).

B. Results

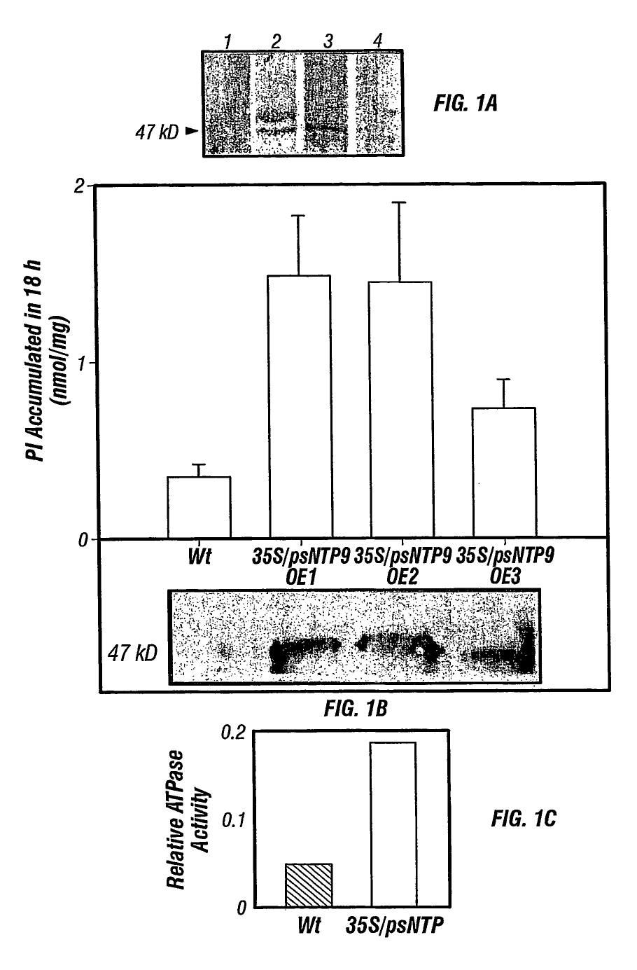

Detection of the pea apyrase in nuclei and in purified plasma membrane: By immunoblot assay, the pea apyrase was found to be associated with nuclei and with purified plasma membranes but not with the cytoplasm (FIG. 1A). The contents of the lanes in FIG. 1A are as follows: Lane 1, cytoplasm; Lane 2, purified plasma membrane; Lane 3, purified nuclei; and Lane 4, pre-immune control of nuclei. Protease treatment destroyed both apyrase activity and antigenicity in outside-out plasma membrane vesicles. After trypsin treatment, the exterior face of the vesicle showed 30% of the ectophosphatase activity of the untreated sample. Endo-phosphatase activities were retained after trypsin treatment, indicating that the digest occurred exclusively on the exterior face of the membrane. These data indicated that the ectoapyrase was in fact being expressed in the extracellular matrix (ECM).

Enhanced Growth of Plants Over-Expressing Apyrase: Three of the four transgenic plant lines constitutively expressed psNTP9 under the control of the cauliflower mosaic virus 35S promoter and over an 18 hour period showed two to five times as much phosphate accumulation in shoots as wild type (FIG. 1B); Top, the total phosphate accumulated in the shoots of three independent transformants in an 18 hour 32 p uptake assay at 2 mM phosphate; Bottom, a corresponding immunoblot performed on equal amounts of protein isolated from the ECM of three week-old wild-type Arabidopsis thaliana and the psNTP9 transgenics. Apyrase expressing plants also showed four times as much phosphatase activity in the extracellular matrix as the wild-type (FIG. 1 C). (Note, OE 1 in the FIG. stands for over-expression 1 transgenic line).

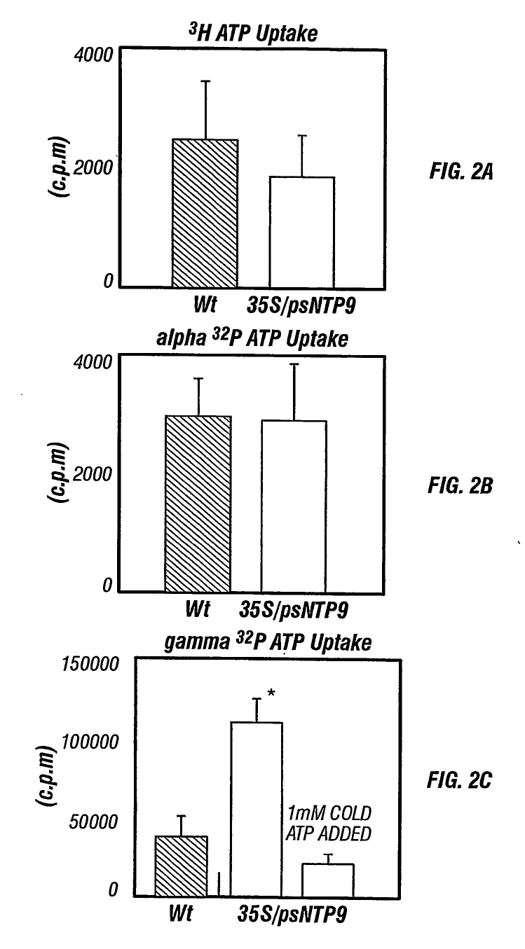

Transgenic plants preferentially transport the gamma phosphate of ATP: In order to address whether over-expression of ectoapyrase was stimulating the adenosine salvage pathway, the intracellular uptake of adenosine was measured both in the presence and absence of the overexpression of apyrase. The inability of apyrase to translocate either extracellular AMP or adenosine was demonstrated by the low level of radiolabel accumulated in the transgenic plants fed [2,8³H]ATP and [a³²P]ATP (FIG. 2). The complete dephosphorylation of [2,8³H]ATP would result in a radiolabelled adenosine molecule while the complete dephosphorylation of [a³²P]ATP would result in a non-labeled adenosine label. FIG. 2A illustrates that plants overexpressing apyrase did not translocate radiolabelled adenosine (or byproducts of the dephosphorylation of [2,8³H]ATP) any more efficiently than plants not overexpressing apyrase (wild-type plants). FIG. 2B illustrates that plants overexpressing apyrase did not translocate AMP (or the byproducts of the dephosphorylated [a³²P]ATP) any more efficiently than wildtype plants. In comparison, feeding experiments where the y phosphate was labeled, the transgenics accumulated three times the amount of labeled phosphate as the wild-type (FIG. 2C). These data show that the over-expression of apyrase does not induce an increase in the uptake of adenosine and therefore its over-expression does not act to stimulate the adenosine salvage pathway.

### Example 4

### EctoPhosphatase is Involved in Drug Resistance in Yeast and Plants

A. Materials and Methods

Expression of AtPGP-1 in yeast: The AtPGP-1 cDNA (Arabidopsis thaliana MDR gene, accession #X61370) was subcloned into pVT101 downstream of the ADH promoter to create the AtPGP-1/pVT101 construct. AtPGP-1/pVT101 and pVT101 were transformed into Saccharomyces cerevisiae INVSCI (genotype: MATa, his3-Δ1, leu2, trpl-289, ura3-52) and YMR4 (genotype: MATαhis3-11,15, leu2-3, 112ura3Δ5, can Res pho5, 3::ura3A1) by a PEG lithium acetate procedure (Eble, 1992) and selected on uracil dropout medium.

Yeast Growth: Yeast were grown at 30° C. under conditions of constant selection for uracil auxotrophy. YNB (Bio101, Vista, Calif.) supplemented with CSM (uracil dropout) and 2% glucose was used to grow strains having pVT101 constructs. Cycloheximide (Sigma Chemical, St. Louis, Mo.) was added to liquid media or spread on solid media to achieve a final concentration of 500 ng/ml. Nigencm (Sigma Chemical, St. Louis, Mo.) was added to liquid media or spread on solid media to achieve a final concentration of 25 μg/ml. Yeast strains used in cycloheximide selection assays were always propagated in the presence of the cycloheximide on plates and then streaked onto new plates containing drug or no drug, such that induced resistance existed in each strain at the time of the start of the assay. For selection assays on plates, single colonies were streaked; for selection in liquid media 0.01 ml of saturated culture was added to fresh media containing the drug. The plates shown in figures were grown for 3-5 days before photographs were taken. Yeast selection assays in liquid media were quantitated by turbidity as measured by absorbance at OD₆₀₀.

Expression of apyrase and AtPGP-1 in plants: The expression of apyrase in plants is as described above in Example 3. Similar methods were employed to express AtPGP-1 in Arabidopsis thaliana plants with the following modifications. The AtPGP-1 coding region was subcloned into a pBIN vector lacking the GUS gene as described in Sidler et al. (1998). This plasmid was then transformed into A. tumefaciens as described above, which was used to infect root calli to produce transgenic plants expressing AtPGP-1.

Plant growth: Arabidopsis thaliana seeds were sown in a solid germination media containing MS salt, 2% sucrose, 0.8% agar, and vitamins (Valvekens et al., 1992). For selection assays, cycloheximide was spread on the media to achieve a final concentration of 250 ng/ml. Plant growth was measured by germination percentage after 6-30 days.

B. Results

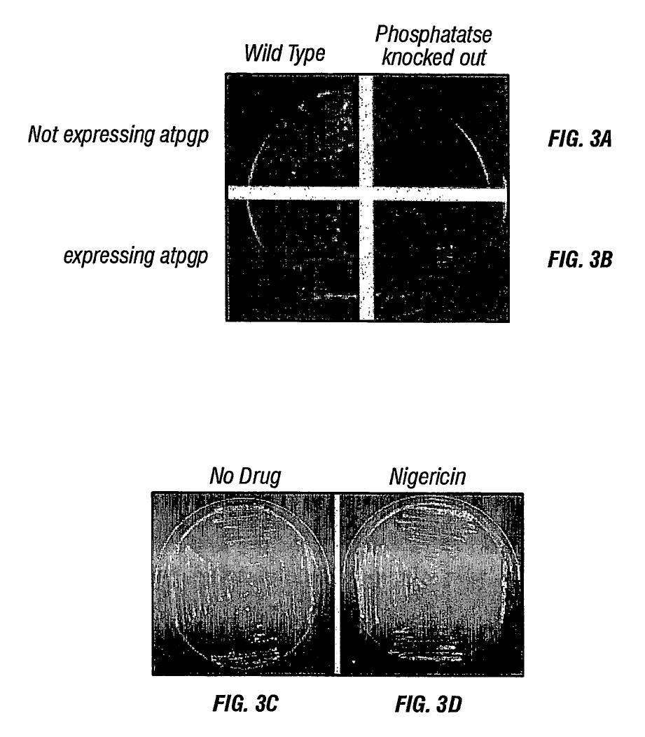

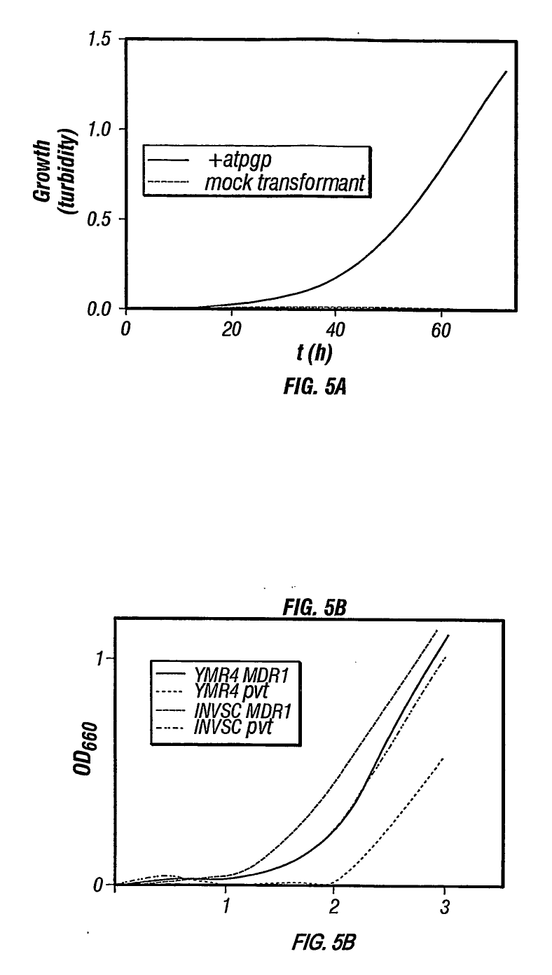

Effect of over-expression of AtPGP-1 in yeast: When a yeast mutant, YNIR4, which is deficient in two major extracellular phosphatases and tends to accumulate ATP extracellularly, was grown in a potent cellular toxin, cycloheximide, it did not grow whereas a wild-type yeast strain, INVSCI, did grow in the presence of cycloheximide (FIG. 3A). Surprisingly, expression of the plant multidrug resistance (MDR) gene, AtPGP-1, enabled the yeast mutant to grow in the toxin (FIG. 3B and FIG. 5A). The presence of AtPGP-1 in the wild-type yeast did not have any effect when grown in the presence of cycloheximide (FIG. 3B). The same result was obtained when the yeast strains were cultured in nigericin (FIG. 3C, 3D, FIG. 5B, 5C). In FIGS. 3C and 3D, starting from the top of the dish clockwise, the cells are as follows: INVSCI (wild-type) overexpressing AtPGP-1, YNM4 containing the vector alone, YMR4 overexpressing AtPGP-1, and INVSCI containing the vector alone. When grown without drug, all the cells grow (FIG. 3C). However, when grown in drug, only the YMR4 containing vector alone shows reduced growth. The survival of the AtPGP-1 transformed strains was due to the ability of the NIDR1 channel to efflux the toxin, hence lowering the actual cellular concentration of the poison cycloheximide. The sensitivity of the untransformed mutant to the drug is likely due to a loss of the ATP gradient below a point at which endogenous transporters, similar to AtPGP-1 can function.

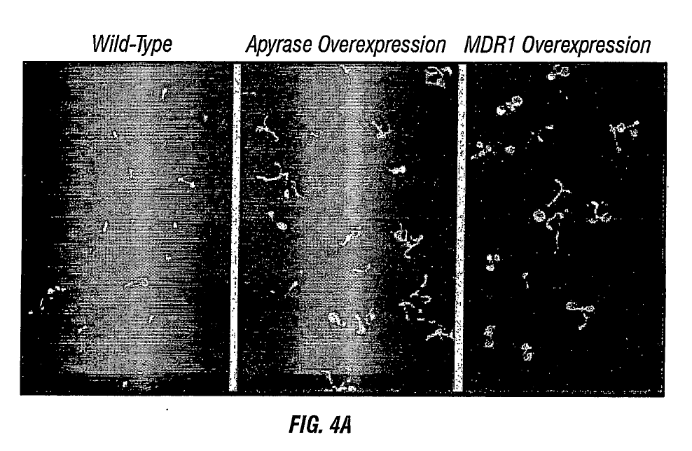

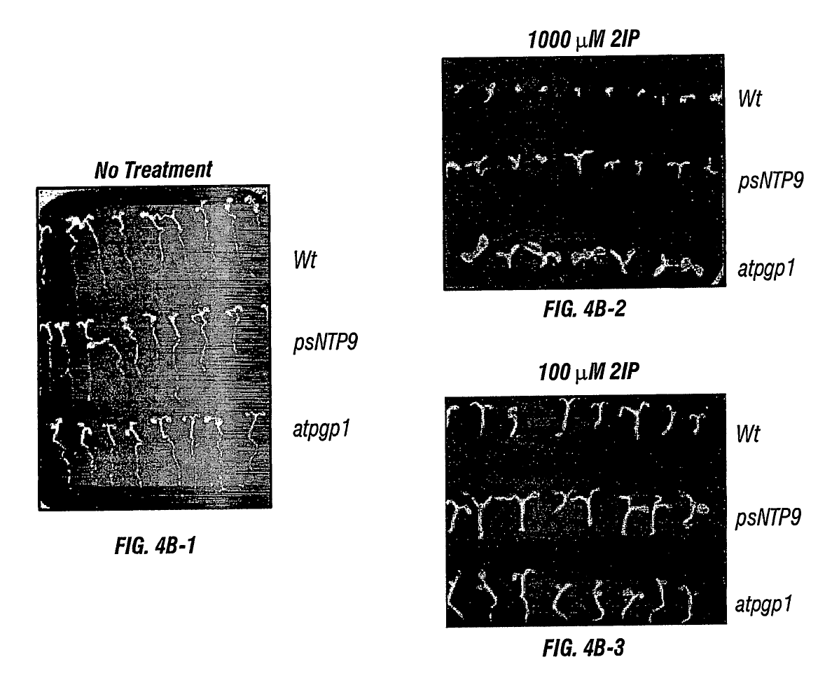

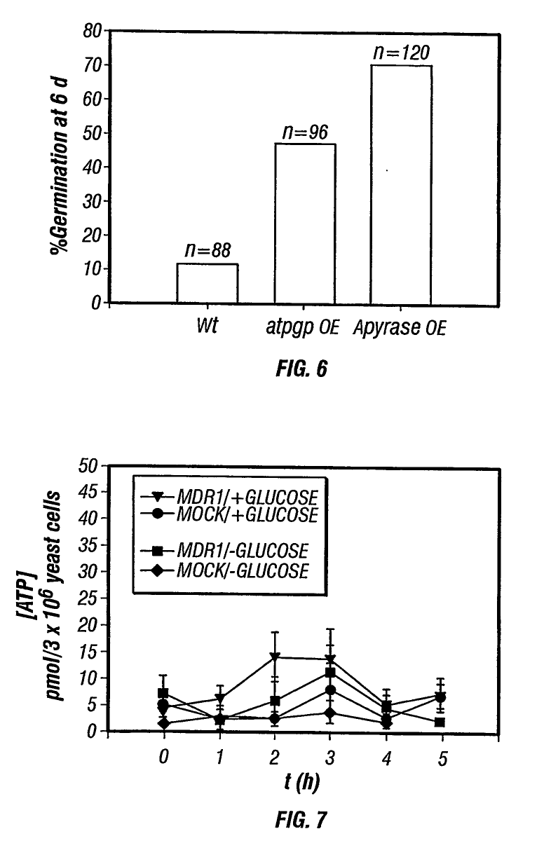

Effect of over-expression of AtPGP-1 in plants: The over-expression of AtPGP-1 was able to confer resistance to cycloheximide in plants (FIGS. 4A and 6) and to the cytokinin, N₆-(2isopentenyl)adenine (21P) (FIG. 4B). These results had not been observed previously and in fact, the prior art actually teaches away from this finding suggesting that over-expression of plant AtPGP-1 is not involved in drug resistance (Sidler. et al., 1998). Therefore, this result was particularly unexpected in plants. Additionally, since Arabidopsis plants overexpressing AtPGP-1 are able to grow in both cycloheximide and cytokinin, this suggests that the conference of drug resistance by AtPGP-1 is likely to be seen with other chemicals as well and is not an isolated phenomenon.

Effect of over-expression of apyrase on drug resistance in plants: Another unexpected result was obtained when the plant apyrase gene was over-expressed in plants. Over-expression of apyrase in plants resulted in the conference of resistance to cycloheximide (FIGS. 4A and 6). The same result was obtained when the plants were grown in the presence of a cytokinin, N₆-(2isopentenyl)adenine (FIG. 4B). In fact, over-expression of apyrase is surprisingly able to raise the germination rate above the level obtained by the over-expression of the MDR gene AtPGP-1 (FIGS. 4A, 4B and 6). Just as under-expression of phosphatase activity in a yeast mutant lacking two potent extracellular phosphatases diminished its resistance to cycloheximide (FIG. 3A), over-expression of a powerful extracellular ATP phosphatase in plants bolstered resistance. The fact that higher resistance was found in plants genetically manipulated only with respect to phosphatase over-expression and not MDR1, indicates that there likely exists other ATP-symporters used in detoxification in addition to MDR1. Minimally, the stronger ATP gradient set up by apyrase in the transgenic plants affects the kinetics of the wild-type MDRI.

### Example 5

### ATP Efflux in Yeast and Plants Overexpressing AtPGP-1

A. Materials and Methods

ATP collection: Yeast cells used in the luciferase assays were grown for two days and then transferred to Lsh media at the time of the assay. From this time forward, the cells were kept at room temperature on a rotator. Every hour a 1 ml aliquot was taken, the cells in the aliquot were counted on a hemocytometer, a methylene blue viability assay was performed (Boyum and Guidotti, 1997), the cells were centrifuged, and the supernatant was stored in liquid nitrogen until all the aliquots were collected. For luciferase assays involving plants, Arabidopsis thaliana plants were grown in sterile culture at 22° C. under 150-200 pE of continuous light for at least 15 days. Foliar ATP was collected by placing a single 30 pl drop of luciferase buffer (Analytical Luminescence Laboratory, Cockeysville, Md.) on a leaf and, without making direct physical contact with the plant, the droplet was immediately collected and snap frozen. For each leaf, the area was approximated as an integrated area of a 2-D image of the leaf using NIH 1.52 software (Shareware, NIH).

Luminometry: Samples were reconstituted to a 100 pl final volume in Firelight™ buffer (Analytical Luminescence Laboratory, Cockeysville, Md.). After the buffer was added, all samples were kept on ice. ATP standards were reconstituted in 100 μl of Firelight™ buffer and the standards and sample were loaded into a 96-well plate and read on an automated Dynex Technologies Model MLX luminometer (Dynex Technologies, Chantilly, Va.). Samples were processed with the addition of 50 μl of Firelight™ enzyme (Analytical Luminescence Laboratory, Cockeysville, Md.) followed by a reading delay of 1.0 second and an integration time of 10 seconds. Output was taken as an average for the integration time and then averaged for multiple samples. The sample handling time was less than 2 hours.

Pulse Chase experiments: Yeast were grown to saturation in liquid medium, as described above, centrifuged, and resuspended in fresh medium containing 1 μCi/ml ³H-adenosine (Amersham, Arlington Heights, Ill.). The cells were rotated at room temperature for 20 minutes to allow adenosine uptake. After 20 minutes the cells were centrifuged. The pellet was washed twice in ice cold medium, resuspended in culture medium at room temperature, divided equally between five types (five per cell line), and placed on a rotator. Every ten minutes a separate tube from each cell line was centrifuged and the pellet and supernatant were placed in separate scintillation vials. The efflux activity was expressed as the ratio of counts in the supernatant to counts in the pellet.

B. Results

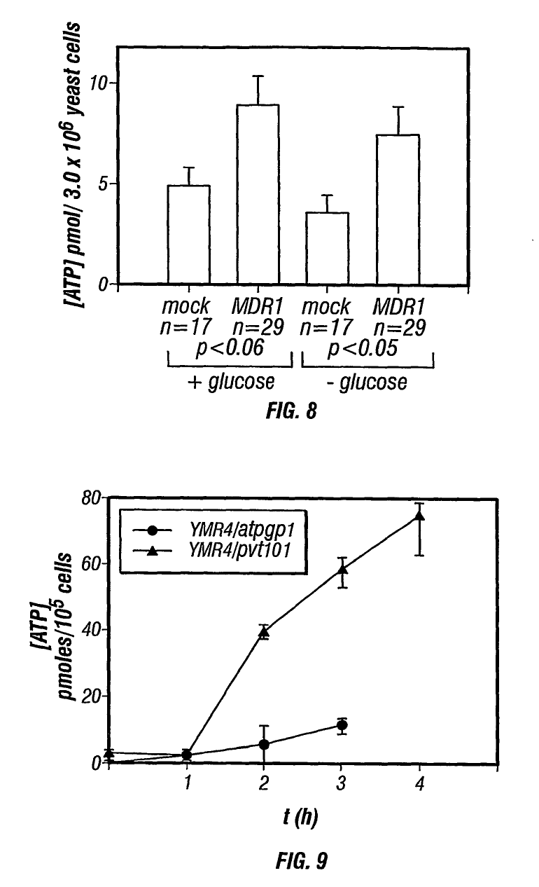

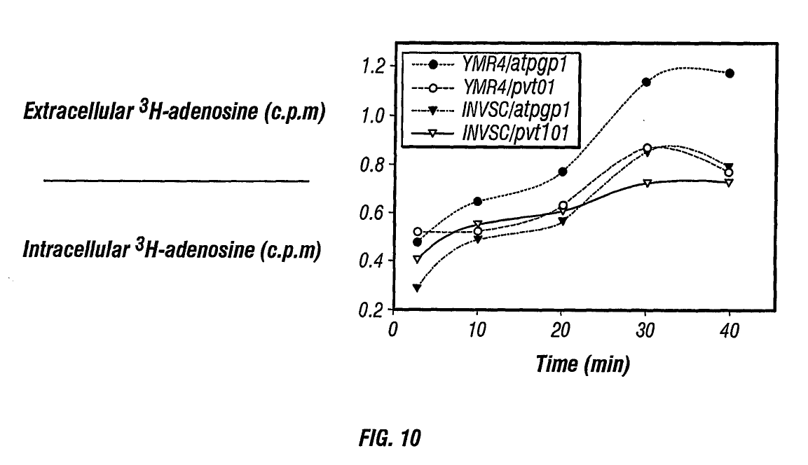

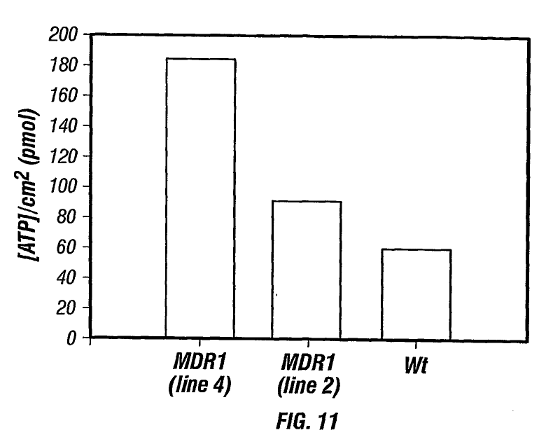

The ATP effluxed by the plant MDR1, AtPGP-1, over-expressed in yeast: In wild-type cells there is a steady-state level of ATP in the extracellular fluid, which is to say that the ATP outside the cells is rapidly degraded by phosphatases and does not accumulate over time (FIG. 7). However, the expression of the AtPGP-1 doubled this steady-state level (FIG. 8). If the yeast mutant, YMR4, which is deficient in extracellular phosphatase activity, is analyzed, there was a noticeable accumulation of ATP in the extracellular fluid compared to a control mutant transformed with empty plasmid pVT101 (FIG. 9). In addition to ATP measurements based on luminometry performed on a kinetic time-scale of hours, an earlier differential ATP efflux in MDRI expressing cells by pulse chase experiments was demonstrated (FIG. 10). Furthermore, Arabidopsis thaliana plants from two independently transformed lines, that constitutively express the AtPGP-1 protein, showed a significant accumulation of ATP on their leaf surfaces (FIG. 11). Taken together, these data demonstrate the absolute ability of plant MDRI, AtPGP1, to transport ATP from inside the cell to the outside. Moreover, these data show that ATP efflux channels and phosphatases both have roles in the steady-state level of ATP outside of the cell. This is the first demonstration of the importance of extracellular ATP steady-state levels, and the importance of an ATP gradient across biological membranes in the modulation of drug resistance.

### Example 6

### A Two-Component System is Found in Arabidopsis Plants

A. Materials and Methods

Plant Growth: Arabidopsis seeds were sown in a solid germination media containing MS salts (Sigma Chemical, St. Louis, Mo.), 2% sucrose, 0.8% agar, and vitamins (Valvekens et al., 1992). For selection assays, one of the following, or a combination of both, was added to media (cooled to less than 50° C. before adding) immediately prior to pouring into plates: cycloheximide at a final concentration of 500 ng/ml; α,β-methyleneadenosine 5′-diphosphate at a final concentration of 1 mM. Plant growth was measured by germination percentage after 10-20 days.

All other materials and methods were discussed above in Example 4.

B. Results

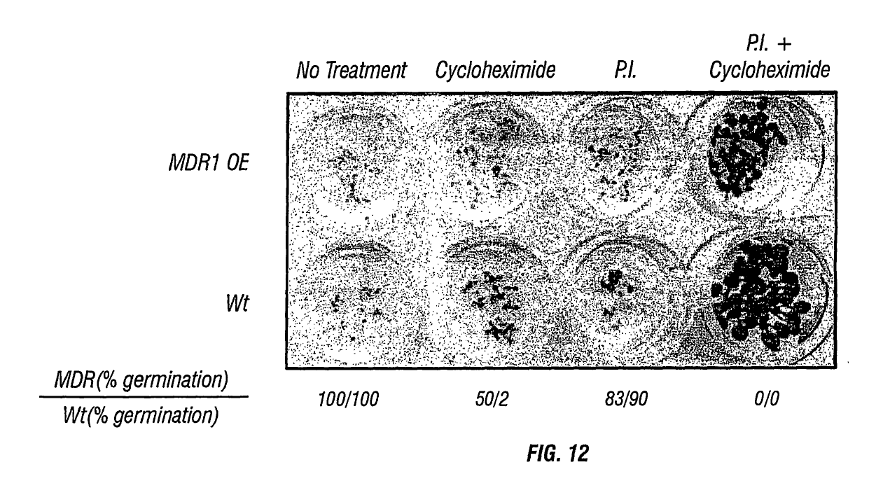

Effects of phosphatase inhibitor on plants overexpressing AtPGP-1: FIG. 12 shows that when wild-type and AtPGP-1 overexpressing (MDR OE) Arabidopsis thaliana plants were either treated with nothing (lane 1), cycloheximide (lane 2), a,(3-methyleneadenosine 5′-diphosphate (phosphatase inhibitor) (lane 3), or cycloheximide and phosphatase inhibitor (lane 4), both the wild-type and the AtPGP-1 overexpressing plants were affected similarly by the presence of phosphatase inhibitor. While the AtPGP-1 overexpressing plants grew significantly better in the presence of cycloheximide alone with a 50% germination rate for the AtPGP-1 overexpressing plants and a 2% germination rate for the wild-type plants, similar germination rates were seen for both the AtPGP-1 overexpressing and wild-type plants in the presence of either phosphatase inhibitor alone (83% and 90% germination respectively) or cycloheximide plus phosphatase inhibitor (no germination at all). The addition of phosphatase inhibitor surprisingly destroys the ability of the AtPGP-expressing plants to grow in the presence of cycloheximide. These data suggest that phosphatases are involved in the conference of drug resistance in plants and that there is a two-component system similar to that demonstrated in yeast in Example 4 and 5 above in which an MDR-like protein and an ATP-gradient-maintaining ectophosphatase are important in modulating drug resistance.

### Example 7

### The ATP Gradient Directly Effects Drug Resistance in Cells

A. Materials and Methods

Cell lines: Cell lines were the same as those described above in Example 4 and 5. YMR4 MDRI is the phosphatase mutant yeast strain overexpressing AtPGP-1; YMR4 pVT011 contains vector alone; INVSC MDRI is the wild-type yeast strain overexpressing AtPGP-1; and INVSC pVT101 contains vector alone.

Selection in drug: To create drug resistant yeast strains, all four cell lines were grown up in the presence of 500 ng/ml of cycloheximide, and transferred to other cycloheximide containing plates after a period of four to six days. This transfer of cell lines and subculturing continued such that the yeast cells grew in the presence of cycloheximide for a period of at least a month. Cells cultured in media alone: To create cell lines that had not been preselected for their ability to grow in drug, yeast strains were grown on plates containing YNB (Bio101, Vista, Calif.) without uracil (-URA) to maintain the presence of the vector (which supplies URA) without any drugs added.

Growth of cells in suspension for ATP and drug selection experiments: Cells were transferred into 5 ml YNB-URA liquid media for turbidity measurements. All cell lines (both non-drug selected and drug-selected) were grown in media with the addition of either nothing, 500 ng/ml cycloheximide, 100 mM ATP, or 500 ng/ml cycloheximide and 100 mM ATP. Turbidity readings were taken after 48 hours.

Growth of cell lines in suspension for salvage pathway experiments: All cell lines were grown in liquid media either containing drug (for the drug selected lines) or not containing drug (for the non-drug selected lines). When the cultures reached a turbidity of 1.00 as measured at a wavelength of 600 in a spectrophotometer (OD₆₀₀=1.00), 10 μl of each culture was then removed and placed in either media with nothing added, 3 mM potassium phosphate; 3 mM adenosine; 9 mM potassium phosphate and 3 mM adenosine (for controls); potassium phosphate and cycloheximide; adenosine and cycloheximide; adenosine, cycloheximide, and potassium phosphate. Cell cultures were further grown for 72 hours, and their turbidity was determined by OD₆₀₀ readings on a spectrophotometer.

Growth of cell lines for nigericin experiments: Drug selected lines were removed from cycloheximide containing plates and placed in 5 ml liquid media containing 5 ng/ml cycloheximide. Cell cultures were allowed to grow until they reached an OD₆₀₀ reading of 1.00, and then 10 μl from each culture was removed and transferred to culture tubes containing 5 ml of liquid media and 25 pg/ml nigericin. OD600 readings were recorded daily for a period of up to 72 hours to determine growth.

B. Results

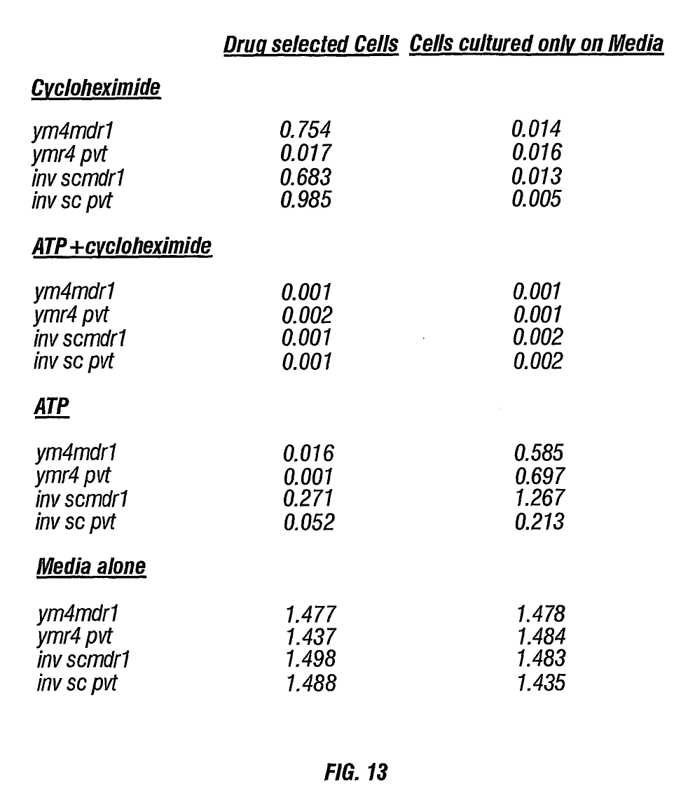

An ATP gradient is critical in MDR: The importance of the ATP gradient in MDR in yeast cells was demonstrated by showing that the growth of cells which were previously grown in drug and had developed resistance to the drug, were not able to grow in high levels of ATP unless they were overexpressing AtPGP-1 (FIG. 13). Cells which had not been previously selected in drug were able to grow in the presence of high levels of ATP (FIG. 13). These data emphasize that the loss of an ATP gradient is previously resistant cell lines abolishes resistance. This result is new to the understanding of MDR and has led to vast insight into the understanding of the mechanism by which MDR-ABC transporters confer resistance to cells and to methods to modulate such resistance. Moreover, when cells were grown in high levels of ATP and drug (cycloheximide), even the cell lines which had previously showed resistance to drug were unable to grow in the presence of drug and ATP. These data indicate that when the ATP gradient across biological membranes is destroyed (by the presence of high extracellular levels of ATP), efflux a of drugs cannot be achieved and therefore, drug resistance is abolished. In summary, the multidrug resistance channel is not functional without an ATP gradient.

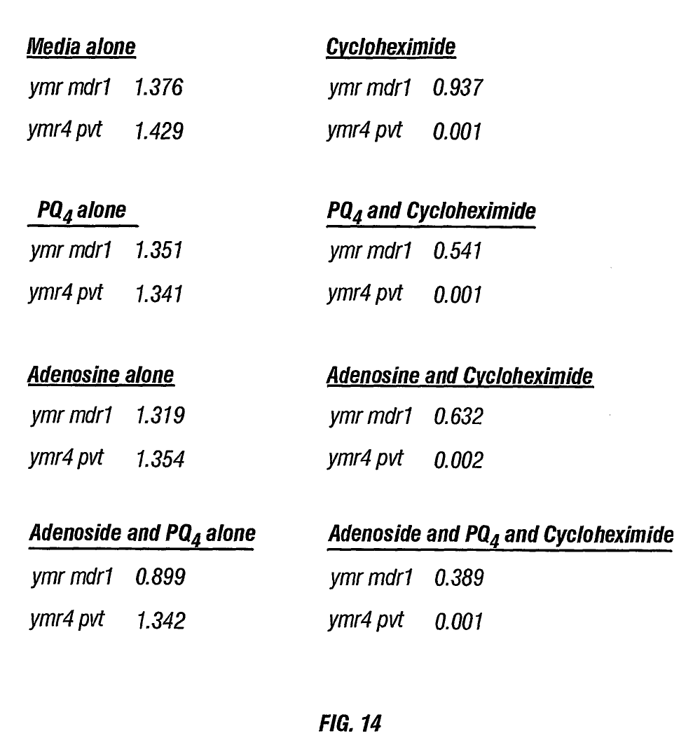

The drug resistance is not due to an adenosine salvage pathway: In order to address whether the involvement of a nucleotide salvage pathway was responsible for the results of the present invention, yeast cells were cultured in the presence of extracellular adenosine and extracellular phosphate. The acid phosphatase yeast mutant, YMR4, was selected because its decreased ectophosphatase activity makes it an ideal candidate for studying the effect of extracellular nucleotides on growth. If an adenosine salvage pathway were involved, then the presence of extracellular adenosine or possibly phosphate should help cells recoup the intracellular ATP losses due to ATP/drug efflux and should help cells grow in the presence of drug whether or not the cells were overexpressing AtPGP-1. In contrast, however, the addition of adenosine or phosphate to the media did not enhance resistance to the cells (FIG. 14). In fact, cells overexpressing AtPGP-1 grew best in drug alone, with the addition of adenosine and/or phosphate being slightly inhibitory. Furthermore, cells which did not express AtPGP-1 were unable to grow in drug regardless of the presence of adenosine and/or phosphate. These data suggest that an adenosine salvage pathway is not the principal mechanism at work in the present invention.

### Example 8

### High Throughput Screen for Isolating Apyrase Inhibitors

A. Materials and Methods

Small Molecule Library: A small molecule library (DIVERSet format F), which was specifically constructed to maximize structural diversity in a relatively small library (9600 compounds), was obtained from ChemBridge Corporation (San Diego, Calif.). The small molecules (supplied in 0.1 mg dehydrated aliquots) were dissolved in DMSO, transferred to a 96 well plate, and tested for their ability to inhibit apyrase activity.

The assay: A stringent screen to test the ability of small molecules to disrupt the ATPase activity of the apyrase enzyme was developed based on phosphate-mobylate complexation. The assay was a modification of a phospholipase assay developed by Hergenrother et al. (1997): Under normal conditions, the apyrase enzyme liberates phosphate from ATP present in the reaction. The liberated phosphate quickly forms a complex upon addition of a small amount of acidified molybdate and ascorbate allowing for the production of a very dark blue color (the less phosphate liberated, the less blue color). Control reactions were performed with heat inactivated apyrase enzyme. Color intensity was detected on an Alpha Imager 2000 with AlphaEase™ software (Alpha Innotech, San Leandro, Calif.). Color changes were also evident by the naked eye. A Biomek 2000 robot (Beckman, Fullerton, Calif.) was used for screening the 9600 samples.

To each well of the 96 well plates containing a small molecule from the library, 100 pI of reaction buffer (60 mM HEPES, 3 MM MgCl₂, 3 mM CaCl₂, 3 mM ATP pH 7.0) was added. The apyrase (potato apyrase grade VI, Sigma Chemical, St. Louis, Mo.) enzyme (0.1 units) was added in a 5 pI volume and the reaction was allowed to proceed at room temperature for 60 minutes.

Three buffers were used to visualize activity: Buffer A: 2% Ammonium molybdate in water Buffer B: 1 I% Ascorbic acid in 37.5% aqueous TCA. Buffer C: 2% trisodium citrate, 2% acetic acid.

Immediately before developing the assay, buffers A and B were mixed in a 1:1 5 ratio. 50 pl of A:B was added to each well. The 96 well plate was then vibrated on a table surface to mix the solution. The deep blue color developed after approximately 2 minutes. After 2 minutes, 50 p.l of buffer C was added to each well and the blue color became darker, increasing the sensitivity of the assay. The color intensified for up to one hour with no accompanying color change in the control wells containing heat inactivated apyrase enzyme. The color intensity for a single plate was measured on an Alpha Imager 2000 with AlphaEase™ software (Alpha Innotech, San Leandro, Calif.).

B. Results:

Nineteen positives were identified from the 9600 compound DIVERSet library. Dose response assays revealed that fourteen showed weak inhibition, two showed medium inhibition (Formulas N and V), and three showed relatively strong inhibition (Formulas I, II and III).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,407,956
U.S. Pat. No. 4,880,918
U.S. Pat. No. 5,705,352
Abraham et al., Proc. Natl. Acad. Sci. USA, 90:312-316, 1993.
Al-Awgati, Science, 269:805-806, 1995.
Baker and Tenover, J. Clin. Microbiol., 34:2654-2659.1996.
Berry-Lowe et al., J. Molec. App. Gen., 1:483-498, 1982.
Binding, In: Plant Protoplasts, CRC Press, Boca Raton, 21-37, 1985.
Bissinger and Kucher, J. Biol. Chem., 269:4180-4186, 1994.
Boyum and Guidotti, Microbiology, 143:1901-1908, 1997.
Che, J. Biol. Chem., 267:9684-9688, 1992.
Dudler and Hertig, J. Biol. Chem., 267:5882-5888, 1992.
Eble, Biotechniques, 13:18-20, 1992.
Fromm et al., Proc. Natl. Acad. Sci. USA, 82:5824-5828, 1985.
Hergenrother et al., Lipids, 32:783788, 1997.
Higgins, Cell, 82:693-696, 1995.
Hooykass et al., In: Molecular Form and Function of the Plant Genome, Plenum Press, N.Y., 655-667, 1984.
Horsch et al., In: Current Communications in Molecular Biology, Cold Spring Harbor Press, Cold Spring Harbor, N.Y., 13-19, 1988.
Huang et al., Biochem. Biophys. Res. Commun., 182:836-843, 1992.
In: Current Protocols in Molecular Biology, John Wiley & Sons, Inc., Mass., USA (1998
Kolacskowski et al., J. Biol. Chem., 271:31543-31548, 1996.
Maniatis et al., In: Molecular Cloning, A Laboratory Manual, Cold Spring Harbor Press, Cold Spring Harbor, N.Y., 1982.
Meyers, Encycl. Molec. Biol. Molec. Med., 6:125-133, 1997.
Paszkowski et al., EMBO J., 3:2717-2722, 1984.
PCT Application WO 98/46736
Plesner, Int. Rev. Cyto., 158:141-214, 1995.
Schardl et al., Gene, 61:1-11, 1987.
Schweibert, Cell, 81:1063-1073, 1995.
Sedaa et al., J. Pharmacol. Exp. Ther., 252:1060-1067, 1990.
Senior et al., FEBS Letters, 377:285-289, 1995.
Sidler et al., The Plant Cell, 10:1623-1636, 1998.
Thomas et al., Plant Cell, 12:519-533, 2000.
Ujhazy et al., Int. J Cancer, 68:493-500, 1996.
Valvekens, Proc. Natl. Acad. Sci. USA, 85:5536-5540, 1992.
Van Veen et al., Nature, 391:291-295, 1998.
Wadkins and Roepe, Int. Rev. Cytology, 171:121-165, 1997.
Wadkins and Roepe, Intl. Rev. Cytology, 171:121-165, 1997.
Wang and Guidotti, J. Biol. Chem., 271:9898-9901, 1996.
Weiner et al. J. Biol. Chem. 261:4529-4534, 1986.
Zimmerman, Biochem. J., 285:345-365, 1992.

1. A cytotoxic composition comprising an ectophosphatase inhibitor and a cytotoxic agent set forth in Table 1.

2. The cytotoxic composition of claim 1, wherein the cytotoxic agent is selectively cytotoxic.

3. The cytotoxic composition of claim 1, further defined as a herbicidal composition and wherein the cytotoxic agent is a herbicide.

4. The cytotoxic composition of claim 1, further defined as an insecticidal composition and wherein the cytotoxic agent is an insecticide.

5. The cytotoxic composition of claim 1, further defined as a fungicidal composition and wherein the cytotoxic agent is a fungicide.

6. The cytotoxic composition of claim 1, further defined as an antibiotic composition and wherein the cytotoxic agent is an antibiotic.

7. The antibiotic composition of claim 6, wherein the antibiotic is from a class selected from the group consisting of Beta-lactam, Semisynthetic penicillin, Clavulanic Acid, Monobactams, Carboxypenems, Aminoglycosides, Glycopeptides, Lincomycins, Macrolides, Polyenes, Rifamycins, Tetracyclines, Semisynthetic, tetracycline and Chloramphenicol.

8. The cytotoxic composition of claim 1, wherein the ectophosphatase inhibitor is selected from the group consisting of the compounds of formulae I-XX.

9. A plant growth regulator composition comprising an ectophosphatase inhibitor and a plant growth regulator agent set forth in Table 1.

10. The plant growth regulator composition of claim 9, wherein the ectophosphatase inhibitor is selected from the group consisting of the compounds of formulae I-XX.

11. A chemotherapeutic composition comprising an ectophosphatase inhibitory compound and a chemotherapeutic agent.

12. The chemotherapeutic composition of claim 11, wherein the chemotherapeutic agent is a chemotherapeutic agent set forth in Table 3.

13. The chemotherapeutic composition of claim 11, wherein the ectophosphatase inhibitor is selected from the group consisting of the compounds of formulae I-XX.

14. A method of killing or inhibiting the growth of a plant, comprising contacting said plant with an effective amount of the composition of claim 3.

15. The method of claim 14, wherein said plant is a monocotyledonous plant.

16. The method of claim 14, wherein the plant is a dicotyledonous plant.

17. A method of killing or inhibiting the growth of a tumor cell, comprising contacting said tumor cell with an effective amount of the composition of claim 11.

18. The method of claim 17, wherein contacting comprises administering said composition of claim 11 to a patient in need thereof, wherein the patient comprises the tumor cell.

19. A method of killing or inhibiting the growth of an insect, comprising contacting said insect with the composition of claim 4.

20. A method of killing or inhibiting the growth of a fungal cell, comprising contacting said cell with the composition of claim 5.

21. A method of killing or inhibiting the growth of a bacterial cell, comprising contacting said cell with the composition of claim 6.

22. A method of increasing the effectiveness of a cytotoxic agent, comprising admixing said cytotoxic agent with an ectophosphatase inhibitor, wherein the cytotoxic agent is selected from the group set forth in Table 1.

23. The method of claim 22, wherein the cytotoxic agent is further defined as a herbicide.

24. The method of claim 22, wherein the cytotoxic agent is further defined as an insecticide, fungicide, or antibiotic.

25-26. (canceled)

27. The method of claim 24, wherein the antibiotic is from a class selected from the group consisting of Beta-lactam, Semisynthetic penicillin, Clavulanic Acid, Monobactams, Carboxypenems, Aminoglycosides, Glycopeptides, Lincomycins, Macrolides, Polyenes, Rifamycins, Tetracyclines, Semisynthetic, tetracycline and Chloramphenicol.

28. The method of claim 22, wherein the ectophosphatase inhibitor is selected from the group consisting of the compounds of formulae I-XX.

29. A method of increasing the effectiveness of a chemotherapeutic agent, comprising admixing said chemotherapeutic agent with an ectophosphatase inhibitor, wherein the chemotherapeutic agent is selected from the group set forth in Table 3.

30. The method of claim 29, wherein the ectophosphatase inhibitor is selected from the group consisting of the compounds of formulae I-XX.
